(19) United States
(12) Patent Application Publication
(10) Pub. No.: US 2024/0166693 A1
(43) Pub. Date: May 23, 2024

(54) METHODS AND COMPOUNDS FOR MODULATING MYOTONIC DYSTROPY 1

(71) Applicant: Design Therapeutics, Inc., Carlsbad, CA (US)

(72) Inventors: Aseem ANSARI, Carlsbad, CA (US); Sean J. JEFFRIES, Carlsbad, CA (US); Pratik SHAH, Carlsbad, CA (US); Chengzhi ZHANG, Carlsbad, CA (US)

(21) Appl. No.: 18/256,864

(22) PCT Filed: Dec. 11, 2021
(86) PCT No.: PCT/US2021/062974

§ 371 Date: Jun. 9, 2023

Related U.S. Application Data
(60) Provisional application No. 63/124,592, filed on Dec. 11, 2020.

(51) Int. Cl. C07K 14/00; A61P 21/00

(52) U.S. Cl. CPC C07K 14/001; A61P 21/00; A61K 38/00

(57) ABSTRACT

The present disclosure relates to transcription modulator molecule compounds and methods for modulating the expression of dmpk, and treating diseases and conditions in which dmpk plays an active role. The transcription modulator comprising a) the first terminus comprising a DNA-binding moiety capable of noncovalently binding to a nucleotide repeat sequence CAG or CTG; b) a second terminus comprising a protein-binding moiety capable of binding to a regulatory molecule that modulates an expression of a gene comprising the nucleotide repeat sequence CAG or CTG; and c) an oligomeric backbone comprising a linker between the first terminus and the second terminus.

### CROSS REFERENCE

This application claims the benefit of U.S. Application No. 63/124,592, filed Dec. 11, 2020, which is hereby incorporated by reference in its entirety.

### FIELD OF THE DISCLOSURE

Disclosed herein are new chimeric heterocyclic polyamide compounds and compositions and their application as pharmaceuticals for the treatment of disease. Methods to modulate the expression of a target gene comprising the CAG trinucleotide repeat sequence in a human or animal subject are also provided for the treatment diseases such as myotonic dystrophy type 1 (“DM1”), spinocerebellar ataxia, Huntington's disease, Huntington's disease-like syndrome, spinobulbar muscular atrophy, and dentatorubral-pallidoluysian atrophy.

### BACKGROUND OF THE DISCLOSURE

The disclosure relates to the treatment of inherited genetic diseases characterized by overproduction of mRNA.

Myotonic dystrophy (“DM”), a member of the class of aliments known as muscular dystrophy, affects approximately 1 in 8000 people. DM is the most common form of muscular dystrophy among adult-onset patients, with most DM cases being diagnosed after age 20. DM is characterized by the persistence of muscular contraction, and is associated with several symptoms, including muscular disorders and cataracts, and cardiac and respiratory disorders, both of which typically are seen later in the progression of the disease. Although treatment is available for the amelioration of associated symptoms, no cure is currently employed that can stop or reverse the progression of DM. Respiratory failure and cardiac dysrhythmia account for the most common causes of death amongst DM patients.

The most severe form of DM is myotonic dystrophy type 1 (“DM1”). DM1 is an autosomal dominant genetic disease, caused by a mutation of the dmpk gene. This gene codes for the myotonic dystrophy protein kinase (MDPK) protein, also known as myotonin-protein kinase. The MDPK protein can be found in muscular, cardiac, and neural tissue.

DM1 is induced by transcription of the defective dmpk gene in DM1 subjects. Normally, this gene contains a 3′ untranslated region with a count of 5-37 CTG trinucleotide repeats. In the DM1 genotype, this trinucleotide is expanded to a count of 50 to over 3,000 repeats, with most having over 1,000 repeats of the CTG sequence. The count tends to increase in descendants, resulting in an earlier age of onset for later generations. Furthermore, the count has been observed to increase in a subject's lifetime, due possibly to aberrant DNA repair.

The progression of DM1 is attributed to “RNA toxicity” from dmpk mRNA having the expanded CTG region. This mRNA forms aggregates with certain proteins, and these aggregates interfere with the normal cellular function. Binding of defective mRNA to muscle blind proteins is perhaps a mechanism leading to the symptoms of DM1, particularly since muscle blind protein activity is required for proper muscle development in flies.

Spinocerebellar ataxia refers to a family of genetic diseases that is characterized by neuronal degeneration, particularly in the cerebellum. Symptoms are generally related to loss of motor function, and include incoordination of gait, poor coordination of manual and eye movements, dysarthria (unclear speech) and related complications such as poor nutrition due to dysphagia.

Several subclasses of spinocerebellar ataxia have been identified, of which a number are linked with the presence of oligonucleotide repeat sequences. Of these, several are associated with multiple copies of the CAG trinucleotide repeat sequence. In six of the subclasses: SCA1, SCA2, SCA3, SCA6, SCA7, and SCA17, the CAG trinucleotide repeat sequences give rise to polyglutamine (poly-Q) repeat sequences in the coded proteins. These subclasses of SCA, along with Huntington's disease, dentatorubral-pallidoluysian atrophy, and spinobulbar muscular atrophy, have been collectively termed “polyglutamine expansion disorders.” The exact mechanism that links the defective poly-Q proteins to the observed pathology is not always clear; aggregation of the protein, as well as formation of ubiquitinated inclusion bodies, has been proposed.

In contrast to the above group of diseases, the CAG trinucleotide repeat sequence of SCA12 is located outside of the coding region of the gene. Thus, although the mRNA contains the CAG trinucleotide repeat sequence, translation of the mRNA does not produce a poly-Q tract. The pathology associated with this defect may be due to the failure of normal cellular mechanisms to break down the abnormal mRNA, perhaps due to the presence of stable hairpin structures, leading to accumulation of the mRNA in the cell.

Spinocerebellar ataxia type 1 (“SCA1”) is associated with the presence of the CAG trinucleotide repeat sequence in the atxn1 gene. Afflicted individuals have 39 or more of the trinucleotide repeat sequence; age of onset of symptoms is inversely correlated with a higher count of trinucleotide repeat sequences. The condition is generally fatal within 10-30 years; no curative treatment is currently available. The CAG trinucleotide repeat sequence is observed in mRNA as well as in genomic DNA. The gene codes for a protein termed ATXN1 which contains a poly-Q tract from the CAG trinucleotide repeat sequences. Animal studies indicate that protein toxicity, and not loss of function, is the primary mechanism responsible for the pathology of defective ATXN1. Degradation of defective ATXN1 by the proteasome is impaired, leading to accumulation of the protein.

Spinocerebellar ataxia type 2 (“SCA2”) is associated with the presence of the CAG trinucleotide repeat sequence in the atxn2 gene. Afflicted individuals have 32 or more of the trinucleotide repeat sequences; age of onset of symptoms is inversely correlated with a higher count of trinucleotide repeat sequences. The gene codes for a protein termed ATXN2 which contains a poly-Q tract from the CAG trinucleotide repeat sequences. The function of the ATXN2 protein is not well understood: it is cytoplasmic and associated with Golgi bodies and the endoplasmic reticulum. Regulation of mRNA translation is suggested by the RNA binding property of ATXN2.

Spinocerebellar ataxia type 3 (“SCA3”) is associated with the presence of the CAG trinucleotide repeat sequence in the atxn3 gene. Afflicted individuals have 50 or more copies of the trinucleotide repeat sequence; age of onset of symptoms is inversely correlated with a higher count of trinucleotide repeat sequences. The gene codes for a protein termed ATXN3 which contains a poly-Q tract from the CAG trinucleotide repeat sequences. The ATXN3 protein plays a role in the ubiquitin/proteasome mechanism for the metabolism of proteins: after a protein is marked for metabolism by ubiquitination, and before degradation of the protein by the proteasome, ATXN3 removes the ubiquitin for recycling. Defective ATXN3 containing a poly-Q tract loses this catalytic property, thus leading to a build-up of unwanted proteins.

Spinocerebellar ataxia type 6 (“SCA6”) is associated with the presence of the CAG trinucleotide repeat sequence in the cacna1a gene. Afflicted individuals have 20 or more of the trinucleotide repeat sequences. Average onset of symptoms is 45 years; the disease progresses slowly, and the duration of the disease can span over 25 years. Treatment for the disease is supportive, with acetazolamide providing relief from ataxia. The gene codes for the alpha-1 subunit of the CaV2.1 calcium channel, which is essential for proper neuronal function. The alpha-1 subunit produced by the defective cacna1a gene in afflicted individuals migrates to the cytoplasm as well as the cell membrane, where it forms aggregates. The mechanism that leads to the observed symptoms is unclear, although malfunction of the calcium channel is suspected, as well as the formation of a toxic C-terminal segment from posttranslational cleavage of the expanded protein.

Spinocerebellar ataxia type 7 (“SCA7”) is associated with the presence of the CAG trinucleotide repeat sequence in the atxn7 gene. Afflicted individuals have from 36 to over 300 of the trinucleotide repeat sequences. Onset of symptoms is typically observed in the second through fourth decade, with earlier onset correlating with more severe symptoms. In addition to the symptoms observed for the SCA class of diseases, subjects with SCA7, particularly subjects with earlier onset, can experience degradation of vision and blindness. Treatment for the disease is supportive only. The gene codes for the ataxin-7 protein, a nuclear protein that plays a role in transcription. In addition, the defective gene product interferes with cone-rod homeobox protein, providing an explanation for the retinopathy observed for this syndrome. Proteolytic cleavage of mutant ataxin-7 and transneuronal responses may be responsible for the pathogenesis of SCA7.

Spinocerebellar ataxia type 17 (“SCA17”) is associated with the presence of the CAG trinucleotide repeat sequence, with CAA interruptions, in the TATA box-binding protein (TBP) gene. The TBP gene product plays a role in the initiation of transcription. Afflicted individuals typically have 47 or more of the trinucleotide repeat sequences. Onset of symptoms is typically observed by age 50, with dysphagia often leading to aspiration and death. The link between the expanded CAG sequence and the observed pathology is unclear, with both gain-of-function and loss-of-function being suggested at different repeat lengths.

Huntington's disease (“HD”) was first identified in the late 19^{th} century as an autosomal dominant, neurodegenerative disorder. The symptoms of HD, which include a range of movement, cognitive and psychiatric disorders, generally appear in adulthood. HD is associated with the presence of the CAG trinucleotide repeat sequence in the htt gene, which codes for a protein termed huntingtin. Subjects with more than about 36 trinucleotide repeat sequences generally present with symptoms of HD, with a larger number of trinucleotide repeat sequences associated with an earlier onset of symptoms. Pathology stems from a cascade of steps: production of poly-Q huntingtin, followed by fragmentation of the elongated huntingtin into smaller peptides, which bind together and accumulate in neurons. The effects of this cascade are pronounced in the basal ganglia and cortex of the brain.

Huntington's disease-like syndrome refers to a group of ailments whose symptoms are similar to those of Huntington's disease, but which lack the characteristic mutation in the htt gene. Huntington's disease-like 2 syndrome (“HDL2”) is associated with count of about 40 or more CAG trinucleotide repeat sequences in the junctophilin 3 (jph3) gene. HDL2 is a genetic disorder that has been seen in subjects with African lineage. Age of onset is inversely correlated with the number of trinucleotide repeat sequences. Symptoms of this syndrome include dystonia and chorea (uncontrolled movements), emotional disruptions, dysarthria, bradykinesia, inability to incorporate new learning, and difficulty in making decisions. Life expectancy can range from a few years post diagnosis to over a decade. The current theory holds that a poly-Q protein that is coded by the jph3 gene forms aggregates in neuronal cells that is responsible for the pathology of the disease. However, evidence suggesting toxic gain-of-function of mRNA has also been uncovered, indicating a possible dual pathway for pathology.

Spinobulbar muscular atrophy, also known as Kennedy disease, is an X-linked genetic disease observed in males whose symptoms include muscle atrophy, dysarthria and dysphagia due to bulbar muscles in the face and throat, fasciculations (involuntary twitches), and infertility. This disease is linked to the presence of the CAG trinucleotide repeat sequences in the androgen receptor (ar) gene. Pathology is thought to be due to the accumulation of fragments of the androgen receptor protein in nerve cells of the brain and spinal cord. Treatment is limited to management of symptoms; neither anti-androgen drugs nor testosterone or analogues display efficacy. Recent studies suggest that pathology of the poly-Q androgen receptor is due to inhibition of the ubiquitin ligase anaphase-promoting complex/cyclostome (APC/C), followed by disruptions in neurite formation and in the cell cycle.

Dentatorubral-pallidoluysian atrophy (“DRPLA”) is an autosomal dominant genetic disorder observed mostly in subjects of Japanese descent whose symptoms, which typically appear in adulthood, include seizures, ataxia, and myoclonus (involuntary spasmodic muscular contractions). Also observed in adult subjects are dementia and psychiatric disorders. DRPLA is linked with a CAG trinucleotide repeat sequence in the atrophin-1 (atn1) gene. Healthy individuals have fewer than about 34 trinucleotide repeat sequences; afflicted individuals generally have more than about 50 trinucleotide repeat sequences. The atrophin-1 protein (“ATN1”) is expressed in all tissue, but is proteolytically cleaved in neurons, suggesting a role in neural activity. Toxicity is thought to be due to accumulation of the ATN1 protein.

Fuchs' endothelial dystrophy of Fuchs' endothelial corneal dystrophy (“FECD”) is a non-inflammatory, sporadic or autosomal dominant, dystrophy involving the endothelial layer of the cornea. With Fuchs' dystrophy the cornea begins to swell causing glare, halos, and reduced visual acuity. The damage to the cornea in Fuchs' endothelial dystrophy can be so severe as to cause corneal blindness. Fuchs' dystrophy has been classified into early-onset (first decade) and late-onset (fourth to the fifth decade) with a predominance of females in the latter. Early-onset Fuchs' has Collagen type 8 α2 chain involvement. Late-onset is characterized by Transcription factor 4, Transcription factor 8 (TCF8), ATP/GTP binding protein-like 1 (AGBL1), lipoxygenase homology domain 1 (LOXHD1), solute carrier family 4 member 11 (SLC4A11) gene and Transforming growth factor-β-induced and clusterin involvement.

In certain embodiments, the mechanism set forth above provides an effective treatment for a disease or disorder which is characterized by the presence of an excessive count of CAG or CTG trinucleotide repeat sequences in a target gene. In some embodiments, the pathology of the disease or disorder is due to the presence of mRNA containing an excessive count of CAG or CTG trinucleotide repeat sequences. In some embodiments, the pathology of the disease or disorder is due to the presence of a translation product containing an excessive count of glutamine amino acid residues. In some embodiments, the pathology of the disease or disorder is due to a loss of function in the translation product. In some embodiments, the pathology of the disease or disorder is due to a gain of function in the translation product. In some embodiments, the pathology of the disease or disorder can be alleviated by increasing the rate of transcription of the defective gene. In some embodiments, the pathology of the disease or disorder can be alleviated by decreasing the rate of transcription of the defective gene.

In certain embodiments, the mechanism set forth above will provide an effective treatment for DM1, which is caused by the overexpression of dmpk. Correction of the overexpression of the defective dmpk gene thus represents a promising method for the treatment of DM1.

### SUMMARY OF THE DISCLOSURE

This disclosure utilizes regulatory molecules present in cell nuclei that control gene expression. Eukaryotic cells provide several mechanisms for controlling gene replication, transcription, and/or translation. Regulatory molecules that are produced by various biochemical mechanisms within the cell can modulate the various processes involved in the conversion of genetic information to cellular components. Several regulatory molecules are known to modulate the production of mRNA and, if directed to the target gene (such as, dmpk, atxn1, atxn2, atxn3, cacna1a, atxn7, ppp2r2b, tbp, htt, jph3, ar, or atn1), would modulate the production of the target gene mRNA that causes diseases such as, for example, spinocerebellar ataxia, Huntington's disease, Huntington's disease-like syndrome, spinobulbar muscular atrophy, and dentatorubral-pallidoluysian atrophy, Fuchs' Endothelial Corneal Dystrophy, and thus reverse the progress of these diseases.

The disclosure provides compounds and methods for recruiting a regulatory molecule into close proximity to the target gene comprising a CAG or CTG trinucleotide repeat sequence. The compounds disclosed herein contain: (a) a DNA binding moiety that will selectively bind to the target gene, optionally linked to (b) a recruiting moiety that will bind to a regulatory molecule. The compounds will counteract the expression of defective target gene in the following manner:
(1) The DNA binding moiety will bind selectively the characteristic CAG trinucleotide repeat sequence of the target gene;
(2) The recruiting moiety, linked to the DNA binding moiety, will thus be held in proximity to the target gene;
(3) The recruiting moiety, now in proximity to the target gene, will recruit the regulatory molecule into proximity with the gene; and
(4) The regulatory molecule will modulate expression of the target gene and therefore counteract the expression of defective mRNA, by direct interaction with the gene.

The DNA binding moiety will bind selectively the characteristic CAG trinucleotide repeat sequence of atxn1, atxn2, atxn3, cacna1a, atxn7, ppp2r2b, tbp, htt, jph3, ar, or atn1 or the DNA binding moiety will bind selectively the characteristic CTG trinucleotide repeat sequence of dmpk. The recruiting moiety, linked to the DNA binding moiety, will thus be held in proximity to the target gene; will recruit the regulatory molecule into proximity with the gene; and the regulatory molecule will modulate expression, and therefore counteract the production of defective target gene by direct interaction with the target gene.

The mechanism provides an effective treatment for DM1, which is caused by the expression of defective dmpk. Additionally, the mechanism provides an effective treatment for spinocerebellar ataxia, Huntington's disease, Huntington's disease-like syndrome, spinobulbar muscular atrophy, and dentatorubral-pallidoluysian atrophy, which are caused by the expression of defective atxn1, atxn2, atxn3, cacna1a, atxn7, ppp2r2b, tbp, htt, jph3, ar, and/or atn1. Correction of the expression of the defective target gene thus represents an effective method for the treatment for these diseases.

The disclosure provides recruiting moieties that bind to regulatory molecules. Small molecule inhibitors of regulatory molecules serve as templates for the design of recruiting moieties, since these inhibitors generally act via noncovalent binding to the regulatory molecules.

The disclosure further provides for DNA binding moieties that selectively bind to one or more copies of the CAG or CTG trinucleotide repeat that are characteristic of the defective target gene. Selective binding of the DNA binding moiety to the target gene, made possible due to the high CAG or CTG count associated with the defective target gene, directs the recruiting moiety into proximity of the gene, and recruits the regulatory molecule into position to modulate gene transcription.

The DNA binding moiety comprises a polyamide segment that will bind selectively to the target CAG or CTG sequence. Polyamides designed by for example Dervan (U.S. Pat. Nos. 9,630,950 and 8,524,899) and others can selectively bind to selected DNA sequences. These polyamides sit in the minor groove of double helical DNA and form hydrogen bonding interactions with the Watson-Crick base pairs. Polyamides that selectively bind to particular DNA sequences can be designed by linking monoamide building blocks according to established chemical rules. One building block is provided for each DNA base pair, with each building block binding noncovalently and selectively to one of the DNA base pairs: A/T, T/A, G/C, and C/G. Following this guideline, trinucleotides binds to molecules with three amide units, i.e. tri-amides. In general, these polyamides can orient in either direction of a DNA sequence.

In principle, longer DNA sequences can be targeted with higher specificity and/or higher affinity by combining a larger number of monoamide building blocks into longer polyamide chains. Ideally, the binding affinity for a polyamide would simply be equal to the sum of each individual monoamide/DNA base pair interaction. In practice, however, due to the geometric mismatch between the fairly rigid polyamide and DNA structures, longer polyamide sequences do not bind to longer DNA sequences as tightly as would be expected from a simple additive contribution. The geometric mismatch between longer polyamide sequences and longer DNA sequences induces an unfavorable geometric strain that subtracts from the binding affinity that would be otherwise expected.

The disclosure, provides for transcription modulator molecules that comprise a DNA binding moiety (for example a polyamide comprising multi-amine subunits) that are connected by flexible spacers (for example a linker moiety that connects the DNA binding moiety to the protein binding moiety). The spacers alleviate the geometric strain that would otherwise decrease binding affinity of a larger polyamide sequences.

Disclosed herein are compound that comprise a polyamide moiety that can bind to one or more copies of the CAG or CTG trinucleotide repeat sequence, and can modulate the expression of a target gene comprising a CAG or CTG trinucleotide repeat sequence. Treatment of a subject with these compounds will modulate expression of the defective target gene, and this can reduce the occurrence, severity, or frequency of symptoms associated with disease. Certain compounds disclosed herein will provide higher binding affinity and selectivity than has been observed previously for this class of compounds.

It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1 shows DM1 fibroblasts (1000 repeats) treatment results after 48 hrs with representative compounds of the disclosure versus Dinaciclib control or no treatment (NT).

FIG. 2 shows DM1 fibroblast treatment results after 6 days with treatment of representative compound of the disclosure. Top panels represent no treatment and bottom panels represent treatment with representative compound in two fibroblast cell lines: GM04602 and GM04647.

### DETAILED DESCRIPTION OF THE DISCLOSURE

The transcription modulator molecule described herein represents an interface of chemistry, biology and precision medicine in that the molecule can be programmed to regulate the expression of a target gene containing nucleotide repeat CAG or CTG. As described herein, “CAG” or “CTG” as used herein refers to the nucleotide CAG and its complementary sequence CTG. A person skilled in the art would understand that a sequence containing CAG trinucleotide (5′-3′ direction) also has CTG trinucleotide on its complementary strand; and a sequence having multiple repeats of CAG in one strand also has multiple repeats of CTG on the complementary strand. Therefore, a polyamide binding to “CAG or CTG” repeat can mean a polyamide binding to CAG and/or its complementary sequence CTG.

The transcription modulator molecule contains DNA binding moieties that will selectively bind to one or more copies of the CAG or CTG trinucleotide repeat that is characteristic of the defective target gene (e.g., dmpk, atxn1, atxn2, atxn3, cacna1a, atxn7, atxn8, atxn80s, ttbk2, ppp2r2b, tbp, htt, jph3, ar, or atn1. The transcription modulator molecule also contains moieties that bind to regulatory proteins. The selective binding of the target gene will bring the regulatory protein into proximity to the target gene and thus downregulates transcription of the target gene. The molecules and compounds disclosed herein provide higher binding affinity and selectivity than has been observed previously for this class of compounds and can be more effective in treating diseases associated with the defective target gene.

Treatment of a subject with these compounds will modulate the expression of the defective target gene, and this can reduce the occurrence, severity, or frequency of symptoms associated with genetic disease (such as for example DM1). The transcription modulator molecules described herein recruits the regulatory molecule to modulate the expression of the defective target gene and effectively treats and alleviates the symptoms associated with diseases.

### Transcription Modulator Molecules

The transcription modulator molecules or compounds disclosed herein possess useful activity for modulating the transcription of a target gene having one or more CAG or CTG repeats (e.g., dmpk or atxn1), and may be used in the treatment or prophylaxis of a disease or condition in which the target gene (e.g., dmpk or atxn1) plays an active role. Thus, in broad aspects, certain embodiments also provide pharmaceutical compositions comprising one or more compounds disclosed herein together with a pharmaceutically acceptable carrier, as well as methods of making and using the compounds and compositions. Certain embodiments provide methods for modulating the expression of dmpk, atxn1, atxn2, atxn3, cacna1a, atxn7, atxn8, atxn80s, ttbk2, ppp2r2b, tbp, htt, jph3, ar, or atn1. Other embodiments provide methods for treating a dmpk, atxn1, atxn2, atxn3, cacna1a, atxn7, atxn8, atxn80s, ttbk2, ppp2r2b, tbp, htt, jph3, ar, or atn1-mediated disorder in a patient in need of such treatment, comprising administering to said patient a therapeutically effective amount of a compound or composition according to the present disclosure. Also provided is the use of certain transcription modulator molecules (i.e., compounds) disclosed herein for use in the manufacture of a medicament for the treatment of a disease or condition ameliorated by the modulation of the expression of dmpk, atxn1, atxn2, atxn3, cacna1a, atxn7, atxn8, atxn80s, ttbk2, tcf4, ppp2r2b, tbp, htt, jph3, ar, or atn1.

In some embodiments, the transcription modulator molecule is a compound having a first terminus, a second terminus, and oligomeric backbone, wherein: a) the first terminus comprises a DNA-binding moiety capable of noncovalently binding to a nucleotide repeat sequence CAG or CTG; b) the second terminus comprises a protein-binding moiety binding to a regulatory molecule that modulates an expression of a gene comprising the nucleotide repeat sequence CAG or CTG; and c) the oligomeric backbone comprising a linker between the first terminus and the second terminus. In some embodiments, the second terminus is not a Brd4 binding moiety. In some embodiments, the nucleotide is CAG. In some embodiments, the nucleotide is CTG.

In some embodiments, the compound has the structural of Formula (I):

X-L-Y Formula (I)

or a salt thereof, wherein:
X comprises a is a recruiting moiety that is capable of noncovalent binding to a regulatory moiety within the nucleus;
Y comprises a DNA recognition moiety that is capable of noncovalent binding to one or more copies of the trinucleotide repeat sequence CAG or CTG; and
L is a linker moiety.

In some embodiments, the regulatory molecule is a polycomb group (PcG) protein. In certain embodiments, the regulatory molecule is a polycomb repressive complex (PRC). In some embodiments, the regulatory molecule is polycomb repressive complex 1 or polycomb repressive complex 2, PRC1 and PRC2 respectively. In some embodiments, the regulator molecule is a polycomb paralog selected from CBX2, CBX4. CBX6, CBX7, and CBX8.

In some embodiments, the first terminus is Y, and the second terminus is X, and the oligomeric backbone is L.

In some embodiments, the compound has the structural Formula (II):

X-L-(Y₁-Y₂-Y₃)ₙ-Y₀ Formula (II)

or a salt thereof, wherein:
X comprises a recruiting moiety that is capable of noncovalent binding to a regulatory molecule within the nucleus;
L is a linker moiety;
Y₁, Y₂, and Y₃ are internal subunits, each of which comprises a moiety chosen from a heterocyclic ring or a C₁₋₆ straight chain aliphatic segment, and each of which is chemically linked to its two neighbors;
Y₀ is an end subunit which comprises a moiety chosen from a heterocyclic ring or a straight chain aliphatic segment, which is chemically linked to its single neighbor;
each subunit can noncovalently bind to an individual nucleotide in the CAG or CTG repeat sequences;
n is an integer between 1 and 15, inclusive; and (Y₁-Y₂-Y₃)ₙ-Y₀ combine to form a DNA recognition moiety that is capable of noncovalent binding to one or more copies of the trinucleotide sequences CAG or CTG.

In some embodiments, the compounds of structural Formula II comprise a subunit for each individual nucleotide in the CAG or CTG repeat sequence. In certain embodiments, the compounds of structural Formula (II) comprise a subunit for each individual nucleotide in the CAG sequence. In certain embodiments, the compounds of structural Formula II comprise a subunit for each individual nucleotide in the CTG repeat sequence.

In some embodiment, each internal subunit has an amino (—NH—) group and a carboxy (—CO—) group.

In certain embodiments, the compounds of structural Formula (II) comprise amide (—NHCO—) bonds between each pair of internal subunits.

In some embodiments, the compounds of structural Formula (II) comprise an amide (—NHCO—) bond between L and the leftmost internal subunit.

In some embodiments, the compounds of structural Formula (II) comprise an amide bond between the rightmost internal subunit and the end subunit.

In certain embodiments, each subunit comprises a moiety that is independently chosen from a heterocycle and an aliphatic chain.

In some embodiments, the heterocycle is a monocyclic heterocycle. In certain embodiments, the heterocycle is a monocyclic 5-membered heterocycle. In certain embodiments, each heterocycle contains a heteroatom independently chosen from N, O, or S. In certain embodiments, each heterocycle is independently chosen from pyrrole, imidazole, triazole, oxazole, thiophene, and furan.

In some embodiments, the aliphatic chain is a C₁₋₆ straight chain aliphatic chain. In certain embodiments, the aliphatic chain has structural formula —(CH₂)ₘ—, for m chosen from 1, 2, 3, 4, and 5. In certain embodiments, the aliphatic chain is —CH₂CH₂—.

In some embodiments, each subunit comprises a moiety independently chosen from

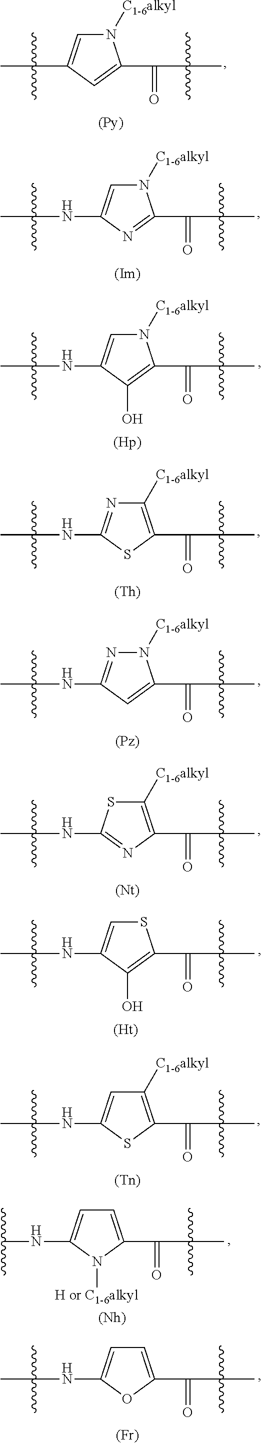

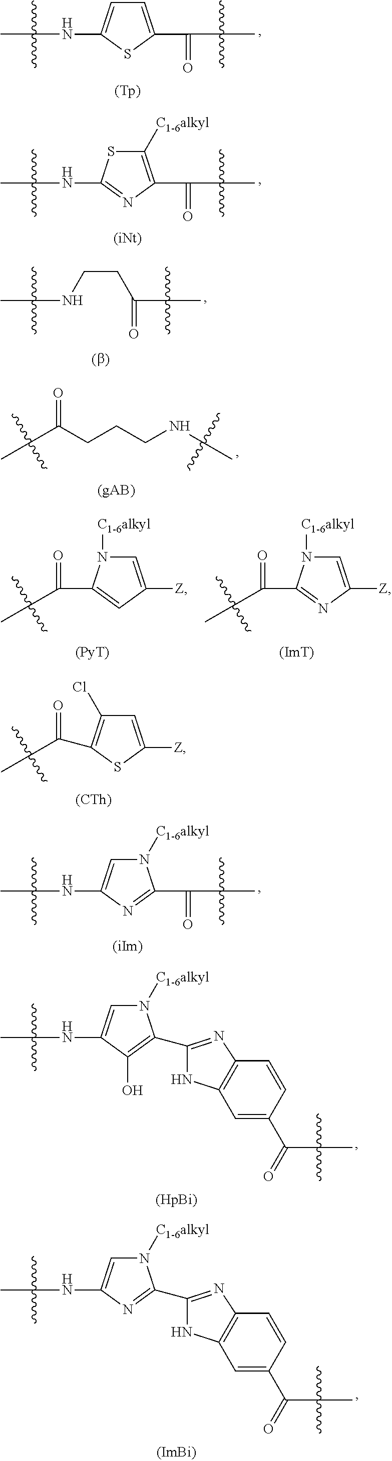

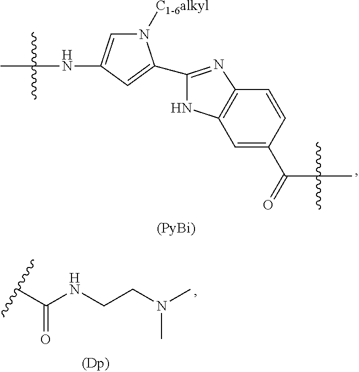

—NH-benzopyrazinylene-CO—, —NH-phenylene-CO—, —NH-pyridiylene-CO—, —NH-piperidinylene-CO—, —NH-pyrimidinylene-CO—, —NH-anthracenylene-CO—, —NH-quinolinylene-CO—, and

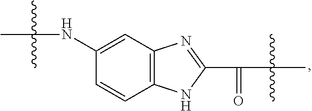

wherein Z is H, NH₂, C₁₋₆ alkyl, C₁₋₆ haloalkyl or C₁₋₆ alkyl-NH₂.

In some embodiments, Py is

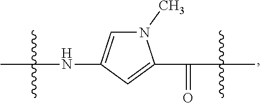 [SMILES: *Nc1cc(C(*)=O)n(C)c1]

### Im is

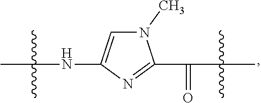 [SMILES: *Nc1cn(C)c(C(*)=O)n1]

### Hp is

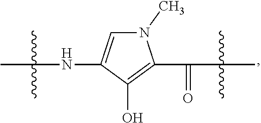 [SMILES: *Nc1cn(C)c(C(*)=O)c1O]

### Th is

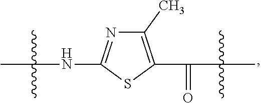 [SMILES: *Nc1nc(C)c(C(*)=O)s1]

### Pz is

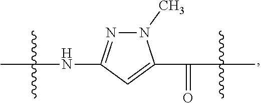 [SMILES: *Nc1cc(C(*)=O)n(C)n1]

### Nt is

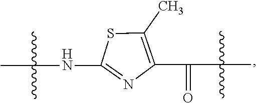

### Tn is

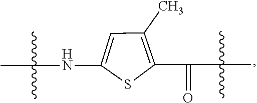

### Nh is

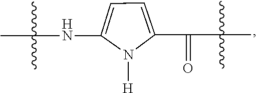

iNt is

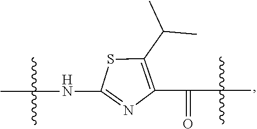

ilm is

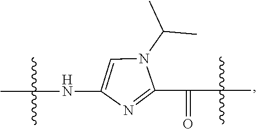

### HpBi is

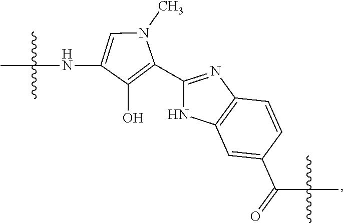

### ImBi is

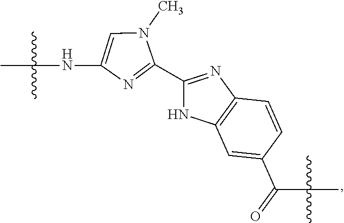

### PyBi is

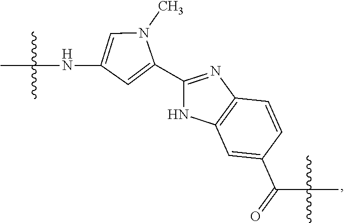

### Dp is

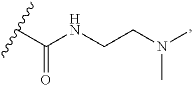

### —NH-benzopyrazinylene-CO— is

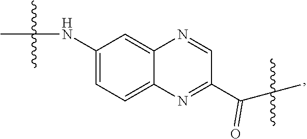

### —NH-phenylene-CO— is

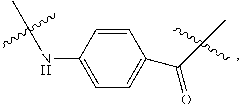 [SMILES: *Nc1ccc(C(*)=O)cc1]

### —NH-pyridiylene-CO— is

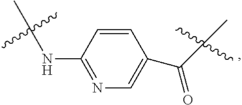 [SMILES: *Nc1ccc(C(*)=O)cn1]

### —NH-piperidinylene-CO— is

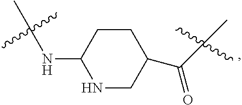 [SMILES: *NC1CCC(C(*)=O)CN1]

### —NH-pyrazinylene-CO— is

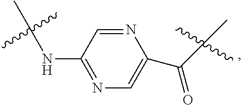 [SMILES: *Nc1cnc(C(*)=O)cn1]

### —NH-anthracenylene-CO— is

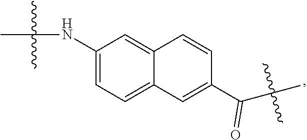

### and —NH-quinolinylene-CO— is

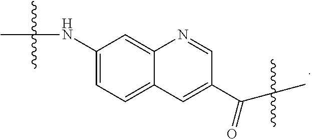 [SMILES: *Nc1ccc2cc(C(*)=O)cnc2c1]

In some embodiments, Py is

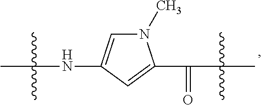 [SMILES: *Nc1cc(C(*)=O)n(C)c1]

### Im is

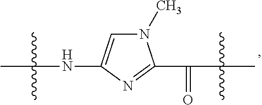 [SMILES: *Nc1cn(C)c(C(*)=O)n1]

### Hp is

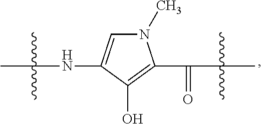 [SMILES: *Nc1cn(C)c(C(*)=O)c1O]

### Th is

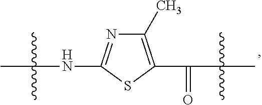 [SMILES: *Nc1nc(C)c(C(*)=O)s1]

### Pz is

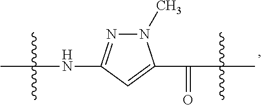 [SMILES: *Nc1cc(C(*)=O)n(C)n1]

### Nt is

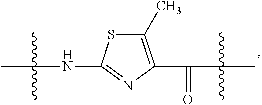 [SMILES: *Nc1nc(C(*)=O)c(C)s1]

### Tn is

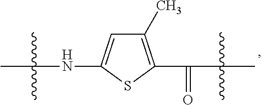 [SMILES: *Nc1cc(C)c(C(*)=O)s1]

### Nh is

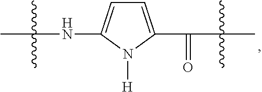 [SMILES: *Nc1ccc(C(*)=O)[nH]1]

iNt is

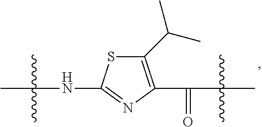 [SMILES: *Nc1nc(C(*)=O)c(C(C)C)s1]

and ilm is

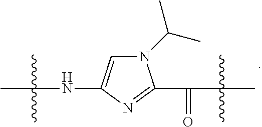 [SMILES: *Nc1cn(C(C)C)c(C(*)=O)n1]

In some embodiments, n is an integer between 1 and 5, inclusive.

In some embodiments, n is an integer between 1 and 3, inclusive.

In some embodiments, n is an integer between 1 and 2, inclusive.

In some embodiments, n is 1.

In some embodiments, L comprises a C₁-C₆ straight chain aliphatic segment.

In some embodiments, L comprises (CH₂OCH₂)ₘ; and m is an integer between 1 to 20, inclusive. In certain further embodiments, m is an integer between 1 to 10, inclusive. In certain further embodiments, m is an integer between 1 to 5, inclusive.

In some embodiments, the compounds has the structure of Formula (III):

X-L-(Y₁-Y₂-Y₃)-(W-Y₁-Y₂-Y₃)ₙ-Y₀ Formula (III)

or a salt thereof, wherein:
X comprises a recruiting moiety that is capable of noncovalent binding to a regulatory molecule within the nucleus;
L is a linker moiety;
Y₁, Y₂, and Y₃ are internal subunits, each of which comprises a moiety chosen from a heterocyclic ring or a C₁₋₆ straight chain aliphatic segment, and each of which is chemically linked to its two neighbors;
Y₀ is an end subunit which comprises a moiety chosen from a heterocyclic ring or a straight chain aliphatic segment, which is chemically linked to its single neighbor;
each subunit can noncovalently bind to an individual nucleotide in the CAG or CTG repeat sequence;
W is a spacer;
n is an integer between 1 and 10, inclusive; and
(Y₁-Y₂-Y₃)-(W-Y₁-Y₂-Y₃)ₙ-Y₀ combine to form a DNA recognition moiety that is capable of noncovalent binding to one or more copies of the trinucleotide repeat sequence CAG or CTG.

In some embodiments, Y₁-Y₂-Y₃ is:

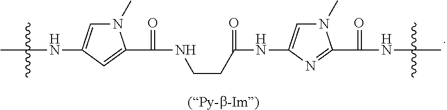 [SMILES: *NC(=O)c1nc(NC(=O)CCNC(=O)c2cc(N*)cn2C)cn1C]

In some embodiments, Y₁-Y₂-Y₃ is Im-β-Py.

In some embodiments, the compound has the structure of Formula (IV):

X-L-(Y₁-Y₂-Y₃)-V-(Y₄-Y₅-Y₆)-Y₀ Formula (IV)

or a salt thereof, wherein:
X comprises a recruiting moiety that is capable of noncovalent binding to a regulatory molecule within the nucleus;
Y₁, Y₂, Y₃, Y₄, Y₅, and Y₆ are internal subunits, each of which comprises a moiety chosen from a heterocyclic ring or a C₁₋₆ straight chain aliphatic segment, and each of which is chemically linked to its two neighbors;
Y₀ is an end subunit which comprises a moiety chosen from a heterocyclic ring or a straight chain aliphatic segment, which is chemically linked to its single neighbor;
each subunit can noncovalently bind to an individual nucleotide in the CAG or CTG repeat sequence;
L is a linker moiety;
V is a turn component for forming a hairpin turn; and
(Y₁-Y₂-Y₃)-V-(Y₄—Y₅-Y₆)-Y₀ combine to form a DNA recognition moiety that is capable of noncovalent binding to one or more copies of the trinucleotide repeat sequence CAG or CTG.

In some embodiments, V is —HN—CH₂CH₂CH₂—CO—.

In some embodiments, the compound has the structure of Formula (Va):

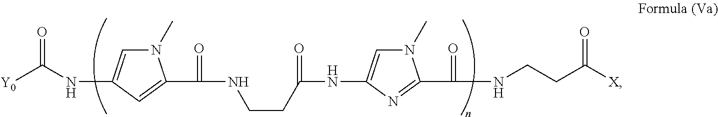

or a salt thereof, wherein:
X comprises a recruiting moiety that is capable of noncovalent binding to a regulatory molecule within the nucleus;
Y₀ is an end subunit which comprises a moiety chosen from a heterocyclic ring or a straight chain aliphatic segment, which is chemically linked to its single neighbor; and
n is an integer between 1 and 5, inclusive.

In some embodiments, the compound has the structure of Formula (VI):

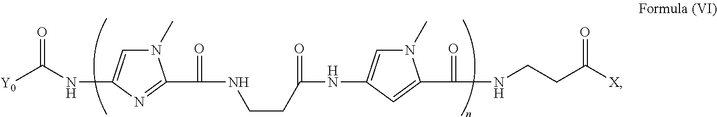

or a salt thereof, wherein:
X comprises a recruiting moiety that is capable of noncovalent binding to a regulatory molecule within the nucleus;
Y₀ is an end subunit which comprises a moiety chosen from a heterocyclic ring or a straight chain aliphatic segment, which is chemically linked to its single neighbor; and
n is an integer between 1 and 5, inclusive.

In some embodiments, the compound has the structure of Formula (VII):

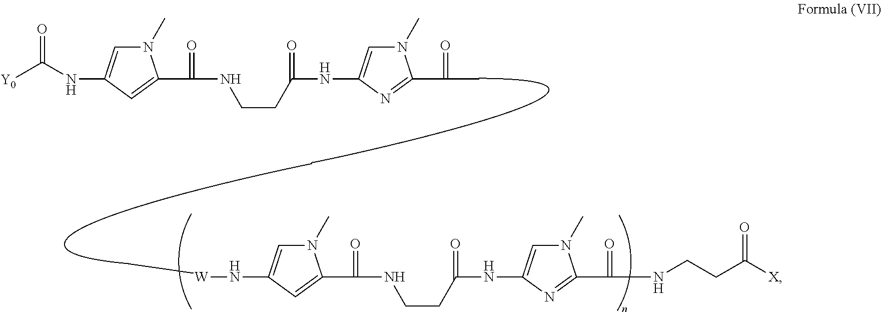

or a salt thereof, wherein:
X comprises a recruiting moiety that is capable of noncovalent binding to a regulatory molecule within the nucleus; and
W is a spacer;
Y₀ is an end subunit which comprises a moiety chosen from a heterocyclic ring or a straight chain aliphatic segment, which is chemically linked to its single neighbor; and n is an integer between 1 and 200, inclusive.

In some embodiments, the compounds have structural Formula (VII):
W is —NHCH₂—(CH₂OCH₂)ₚ—CH₂CO—; and
p is an integer between 1 and 4, inclusive

In some embodiments, the compound has the structure of Formula (VIII):

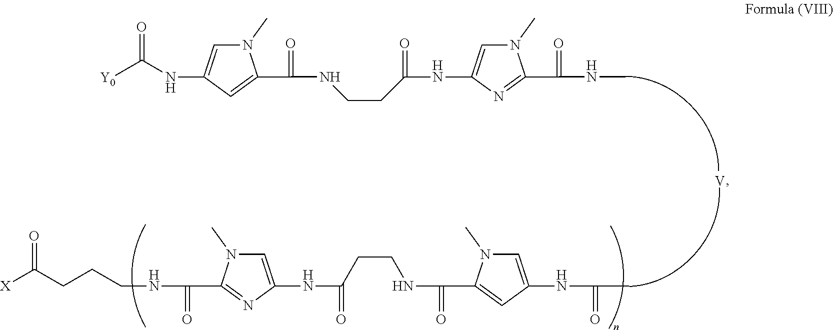

or a salt thereof, wherein:
X comprises a recruiting moiety that is capable of noncovalent binding to a regulatory molecule within the nucleus;
V is a turn component for forming a hairpin turn;
Y₀ is an end subunit which comprises a moiety chosen from a heterocyclic ring or a straight chain aliphatic segment, which is chemically linked to its single neighbor; and
n is an integer between 1 and 200, inclusive.

In some embodiments of the compounds of structural Formula (VIII), V is —(CH₂)q-NH—(CH₂)_{q}—; and q is an integer between 2 and 4, inclusive.

In some embodiments, V is —(CH₂)ₐ—NR¹—(CH₂)_{b}—, —(CH₂)ₐ—, —(CH₂)ₐ—O—(CH₂)_{b}—, —(CH₂)ₐ—CH(NHR¹)—, —(CH₂)ₐ—CH(NHR¹)—, —(CR²R³)ₐ—, or —(CH₂)ₐ—CH(NR¹₃)⁺—(CH₂)_{b}—, wherein each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, R¹ is H. In some embodiments, R¹ is C₁₋₆ alkyl optionally substituted by 1-3 substituents selected from —C(O)-phenyl. In some embodiments, V is —(CR²R³)—(CH₂)a- or —(CH₂)ₐ—(CR²R³)—(CH₂)_{b}—, wherein each a is independently 1-3, b is 0-3, and each R² and R³ are independently H, halogen, OH, NHAc, or C₁₋₄ alky. In some embodiments, V is —(CH₂)—CH(NH₃)⁺—(CH₂)— or —(CH₂)—CH₂CH(NH₃)—.

In one aspect, the compounds of the present disclosure bind to the CAG or CTG of dmpk, atxn1, atxn2, atxn3, cacna1a, atxn7, atxn8, atxn80s, ttpk2, tcf4, ppp2r2b, tbp, htt, jph3, ar, or atn1 and recruit a regulatory moiety to the vicinity of dmpk, atxn1, atxn2, atxn3, cacna1a, atxn7, ppp2r2b, tbp, htt, jph3, ar, or atn1. The regulatory moiety, due to its proximity to the gene, will be more likely to modulate the expression of dmpk, atxn1, atxn2, atxn3, cacna1a, atxn7, atxn8, atxn80s, ttpk2, tcf4, ppp2r2b, tbp, htt, jph3, ar, or atn1.

Also provided are embodiments wherein any compound disclosed above, including compounds of Formulas (I)-(VIII), are singly, partially, or fully deuterated. Methods for accomplishing deuterium exchange for hydrogen are known in the art.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

In one aspect, the compounds of the present disclosure bind to the CAG or CTG of dmpk, atxn1, atxn2, atxn3, cacna1a, atxn7, atxn8, atxn80s, ttpk2, tcf4, ppp2r2b, tbp, htt, jph3, ar, or atn1 and recruit a regulatory moiety to the vicinity of dmpk, atxn1, atxn2, atxn3, cacna1a, atxn7, ppp2r2b, tbp, htt, jph3, ar, or atn1. The regulatory moiety, due to its proximity to the gene, will be more likely to modulate the expression of dmpk, atxn1, atxn2, atxn3, cacna1a, atxn7, atxn8, atxn80s, ttbk2, tcf4, ppp2r2b, tbp, htt, jph3, ar, or atn1.

In some embodiments, the molecules described herein bind to the CAG of atxn1, atxn2, atxn3, cacna1a, atxn7, atxn8, atxn80s, ttbk2, tcf4, ppp2r2b, tbp, htt, jph3, ar, and atn1. In some embodiments, the molecules described herein bind to the CAG of the gene encoding TCF4. In some embodiments, the molecules of the present disclosure bind to the CTG of dmpk. In some embodiments, the molecules of the present disclosure bind to the CAG of TCF4 gene.

In one aspect, the molecules of the present disclosure provide a polyamide sequence for interaction of a single polyamide subunit to each base pair in the CAG or CTG repeat sequence. In one aspect, the molecules of the present disclosure provide a turn component V, in order to enable hairpin binding of the molecule to the CAG or CTG, in which each nucleotide pair interacts with two subunits of the polyamide.

In one aspect, the molecules of the present disclosure provide more than one copy of the polyamide sequence for noncovalent binding to the CAG or CTG. In one aspect, the molecules of the present disclosure bind to dmpk, atxn1, atxn2, atxn3, cacna1a, atxn7, atxn8, atxn80s, ttbk2, tcf4, ppp2r2b, tbp, htt, jph3, ar, or atn1 with an affinity that is greater than a corresponding molecule that contains a single polyamide sequence.

In one aspect, the molecules of the present disclosure provide more than one copy of the polyamide sequence for noncovalent binding to the CAT or CTG, and the individual polyamide sequences in this molecule are linked by a spacer W, as defined above. The spacer W allows this molecule to adjust its geometry as needed to alleviate the geometric strain that otherwise affects the noncovalent binding of longer polyamide sequences.

### First Terminus DNA Binding Moiety

The first terminus interacts and binds with the gene, particularly with the minor grooves of the CAG or CTG sequence. In one aspect, the molecules of the present disclosure provide a polyamide sequence for interaction of a single polyamide subunit to each base pair in the CAG or CTG repeat sequence. In one aspect, the molecules of the present disclosure provide a turn component (e.g, aliphatic amino acid moiety), in order to enable hairpin binding of the molecule to the CAG or CTG, in which each nucleotide pair interacts with two subunits of the polyamide.

In one aspect, the molecules of the present disclosure provide more than one copy of the polyamide sequence for noncovalent binding to CAG or CTG. In one aspect, the molecules of the present disclosure bind to dmpk, atxn1, atxn2, atxn3, cacna1a, atxn7, atxn8, atxn80s, ttbk2, tcf4, ppp2r2b, tbp, htt, jph3, ar, or atn1 with an affinity that is greater than a corresponding molecule that contains a single polyamide sequence. In one aspect, the molecules of the present disclosure bind to dmpk, atxn1, atxn2, atxn3, cacna1a, atxn7, ppp2r2b, tbp, htt, jph3, ar, or atn1 with an affinity that is greater than a corresponding molecule that contains a single polyamide sequence.

In one aspect, the molecules of the present disclosure provide more than one copy of the polyamide sequence for noncovalent binding to the CAG or CTG, and the individual polyamide sequences in this molecule are linked by a spacer W, as defined above. The spacer W allows this molecule to adjust its geometry as needed to alleviate the geometric strain that otherwise affects the noncovalent binding of longer polyamide sequences.

In some embodiments, the DNA recognition or binding moiety binds in the minor groove of DNA.

In some embodiments, the DNA recognition or binding moiety comprises a polymeric sequence of monomers, wherein each monomer in the polymer selectively binds to a certain DNA base pair.

In some embodiments, the DNA recognition or binding moiety comprises a polyamide moiety.

In certain embodiments, the DNA recognition or binding moiety comprises a polyamide moiety comprising heteroaromatic monomers, wherein each heteroaromatic monomer binds noncovalently to a specific nucleotide, and each heteroaromatic monomer is attached to its neighbor or neighbors via amide bonds.

In some embodiments, the DNA recognition moiety binds to a sequence comprising at least 1000 nucleotide repeats. In certain embodiments, the DNA recognition moiety binds to a sequence comprising at least 500 nucleotide repeats. In certain embodiments, the DNA recognition moiety binds to a sequence comprising at least 200 nucleotide repeats. In certain embodiments, the DNA recognition moiety binds to a sequence comprising at least 100 nucleotide repeats. In certain embodiments, the DNA recognition moiety binds to a sequence comprising at least 50 nucleotide repeats. In certain embodiments, the DNA recognition moiety binds to a sequence comprising at least 20 nucleotide repeats.

In certain embodiments, each subunit comprises a moiety that is independently chosen from a heterocycle and an aliphatic chain.

In certain embodiments, the heterocycle is a monocyclic heterocycle. In certain embodiments, the heterocycle is a monocyclic 5-membered heterocycle. In certain embodiments, each heterocycle contains a heteroatom independently chosen from N, O, or S. In certain embodiments, each heterocycle is independently chosen from pyrrole, imidazole, thiazole, oxazole, thiophene, and furan.

In certain embodiments, the aliphatic chain is a C₁₋₆ straight chain aliphatic chain. In certain embodiments, the aliphatic chain has structural formula —(CH₂)ₘ—, for m chosen from 1, 2, 3, 4, and 5. In certain embodiments, the aliphatic chain is —CH₂CH₂—.

In some embodiments, the first terminus comprises —NH-Q-C(O)—, wherein Q is an optionally substituted C₆₋₁₀ arylene group, optionally substituted 4-10 membered heterocyclene, optionally substituted 5-10 membered heteroarylene group, or an optionally substituted alkylene group. In some embodiments, Q is an optionally substituted C₆₋₁₀ arylene group or optionally substituted 5-10 membered heteroarylene group. In some embodiments, Q is an optionally substituted 5-10 membered heteroarylene group. In some embodiments, the 5-10 membered heteroarylene group is optionally substituted with 1-4 substituents selected from H, OH, halogen, C₁₋₁₀ alkyl, NO₂, CN, NR′R″, C₁₋₆ haloalkyl, C₁₋₆ alkoxyl, C₁₋₆haloalkoxy, (C₁₋₆ alkoxy) C₁₋₆ alkyl, C₂₋₁₀ alkenyl, C₂₋₁₀ alkynyl, C₃₋₇ carbocyclyl, 4-10 membered heterocyclyl, C₆₋₁₀ aryl, 5-10 membered heteroaryl, (C₃₋₇carbocyclyl)C₁₋₆ alkyl, (4-10 membered heterocyclyl)C₁₋₆ alkyl, (C₆₋₁₀ aryl)C₁₋₆ alkyl, (C₆₋₁₀ aryl)C₁₋₆ alkoxy, (5-10 membered heteroaryl)C₁₋₆ alkyl, (C₃₋₇carbocyclyl)-amine, (4-10 membered heterocyclyl)amine, (C₆₋₁₀aryl)amine, (5-10 membered heteroaryl)amine, acyl, C-carboxy, O-carboxy, C-amido, N-amido, S-sulfonamido, N-sulfonamido, —SR, COOH, or CONR′R″; wherein each R′ and R″ are independently H, C₁₋₁₀ alkyl, C₁₋₁₀ haloalkyl, C₁₋₁₀ alkoxyl.

In some embodiments, the first terminus comprises at least three aromatic carboxamide moieties selected to correspond to the nucleotide repeat sequence CAG or CTG and at least one aliphatic amino acid residue chosen from the group consisting of glycine, β-alanine, γ-aminobutyric acid, 2,4-diaminobutyric acid, and 5-aminovaleric acid. In some embodiments, the first terminus comprises at least one β-alanine subunit.

In some embodiments, the monomer element is independently selected from the group consisting of optionally substituted pyrrole carboxamide monomer, optionally substituted imidazole carboxamide monomer, optionally substituted C—C linked heteromonocyclic/heterobicyclic moiety, and β-alanine. In some embodiments, the first terminus comprises one or more subunits selected from the group consisting of optionally substituted N-methylpyrrole, optionally substituted N-methylimidazole, and β-alanine (β).

The form of the polyamide selected can vary based on the target gene. The first terminus can include a polyamide selected from the group consisting of a linear polyamide, a hairpin polyamide, a H-pin polyamide, an overlapped polyamide, a slipped polyamide, a cyclic polyamide, a tandem polyamide, and an extended polyamide. In some embodiments, the first terminus comprises a linear polyamide. In some embodiments, the first terminus comprises a hairpin polyamide.

The binding affinity between the polyamide and the target gene can be adjusted based on the composition of the polyamide. In some embodiments, the polyamide is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the polyamide is capable of binding the DNA with an affinity of less than about 300 nM. In some embodiments, the polyamide is capable of binding the DNA with an affinity of less than about 200 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, or 100-300 nM.

The binding affinity between the polyamide and the target DNA can be determined using a quantitative footprint titration experiment. The experiment involve measuring the dissociation constant K_{d} of the polyamide for target sequence at either 24° C. or 37° C., and using either standard polyamide assay solution conditions or approximate intracellular solution conditions.

The binding affinity between the regulatory protein and the ligand on the second terminus can be determined using an assay suitable for the specific protein. The experiment involve measuring the dissociation constant K_{d} of the ligand for protein and using either standard protein assay solution conditions or approximate intracellular solution conditions.

In some embodiments, the first terminus comprises a structure of Formula (A-1), or a pharmaceutically acceptable salt or solvate thereof:

-L₁ₐ-[A-M]ₚ₁-L₂ₐ-[M-A]_{q1}-E₁ Formula (A-1),

wherein:
L₁ₐ is a bond, a C₁₋₆ alkylene, —NH—C₀₋₆ alkylene-C(O)—, —N(CH₃)—C₀₋₆ alkylene, or —O—C₀₋₆ alkylene;
L₂ₐ is a bond, C₁₋₆ alkylene, —NH—C₀₋₆ alkylene-C(O)—, —N(CH₃)—C₀₋₆ alkylene, —O—C₀₋₆ alkylene, —(CH₂)ₐ₁—NR^{a}—(CH₂)_{b1}—, —(CH₂)ₐ—, —(CH₂)ₐ₁—O—(CH₂)_{b1}—, —(CH₂)ₐ₁—CH(NHR^{a})—, —(CH₂)ₐ₁—CH(NHR^{a})—, or —(CH₂)ₐ₁—CH(NR^{a}R^{b})—(CH₂)_{b1}—;
each a¹ and b¹ are independently an integer between 2 and 4;
each R^{a} and R^{b} are independently selected from H, an optionally substituted C₁₋₆ alkyl, an optionally substituted C₃₋₁₀ cycloalkyl, optionally substituted C₆₋₁₀ aryl, optionally substituted 4-10 membered heterocyclyl, and an optionally substituted 5-10 membered heteroaryl;
each [A-M] appears p¹ times and p¹ is an integer in the range of 1 to 10;
each [M-A] appears q¹ times and q¹ is an integer in the range of 1 to 10;
each A is selected from a bond, C₁₋₁₀ alkylene, optionally substituted C₆₋₁₀ arylene group, optionally substituted 4-10 membered heterocyclene, optionally substituted 5-10 membered heteroarylene group, —C₁₋₁₀ alkylene-C(O)—, —C₁₋₁₀ alkylene-NR^{a}—, —CO—, —NR^{a}—, —CONR^{a}—, —CONR^{a}C₁₋₄alkylene-, —NR^{a}CO—C₁₋₄alkylene-, —C(O)O—, —O—, —S—, —C(═S)—NH—, —C(O)—NH—NH—, —C(O)—N═N—, —C(O)—CH═CH—, (CH₂)₀₋₄—CH═CH—(CH₂)₀₋₄, —N(CH₃)—C₁₋₆ alkylene,

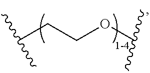

—NH—C₁₋₆ alkylene-NH—, —O—C₁₋₆ alkylene-O—, —NH—N═N—, —NH—C(O)—NH—, and any combinations thereof, and at least one A is —CONH—;
each M in each [A-M] and [M-A] unit is independently an optionally substituted C₆₋₁₀ arylene group, optionally substituted 4-10 membered heterocyclene, optionally substituted 5-10 membered heteroarylene group, or an optionally substituted alkylene; and
E₁ is selected from the group consisting of optionally substituted C₆₋₁₀ aryl, optionally substituted 4-10 membered heterocyclyl, optionally substituted 5-10 membered heteroaryl, an optionally substituted C₁₋₆ alkyl, C₀₋₄ alkylene-NHC(═NH)NH, —CN, —C₀₋₄alkylene-C(═NH)(NR^{a}R₂), —C₀₋₄alkylene-C(═N⁺H₂)(NR^{a}R^{b}) C1-5alkylene-NR^{a}R^{b}, C₀₋₄alkylene-NHC(═NH) R^{a}, and optionally substituted amine.

In certain embodiments, the integers p¹ and q¹ are 2≤p¹+q¹≤20.

In some embodiments of Formula (A-1), each A is independently a bond, C₁₋₆ alkylene, optionally substituted phenylene, optionally substituted thiophenylene, optionally substituted furanylene, —C₁₋₁₀ alkylene-C(O)—, —C₁₋₁₀ alkylene-NH—, —CO—, —NR^{a}—, —CONR^{a}—, —CONR^{a}C₁₋₄alkylene-, —NR^{a}CO—C₁₋₄alkylene-, —C(O)O—, —O—, —S—, —S(O)—, —S(O)₂—, —C(═S)—NH—, —C(O)—NH—NH—, —C(O)—N═N—, —C(O)—CH═CH—, —CH═CH—, —NH—N═N—, —NH—C(O)—NH—, —N(CH₃)—C₁₋₆ alkylene, and

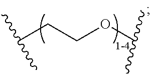

—NH—C₁₋₆ alkylene-NH—, —O—C₁₋₆ alkylene-O—, and any combinations optionally substituted 5-10 membered heteroarylene group. In some embodiments of Formula (A-1), L₁ₐ is a bond. In some embodiments of Formula (A-1), L₁ₐ is a C₁₋₆ alkylene. In some embodiments of Formula (A-1), L₁ₐ is —NH—C₁₋₆ alkylene-C(O)—. In some embodiments of Formula (A-1), L₁ₐ is —N(CH₃)—C₁₋₆ alkylene-. In some embodiments, in Formula (A-1), L₁ₐ is —O—C₀₋₆ alkylene-.

In some embodiments, L₃ₐ is a bond. In some embodiments, L₃ₐ is C₁₋₆ alkylene. In some embodiments, L₃ₐ is —NH—C₁₋₆ alkylene-C(O)—. In some embodiments, L₃ₐ is —N(CH₃)—C₁₋₆ alkylene-C(O)—. In some embodiments, L₃ₐ is —O—C₀₋₆ alkylene. In some embodiments, L₃ₐ is —(CH₂)ₐ—NR^{a}—(CH₂)_{b}—. In some embodiments, L₃ₐ is —(CH₂)ₐ—O—(CH₂)_{b}—. In some embodiments, L₃ₐ is —(CH₂)ₐ—CH(NHR^{a})—. In some embodiments, L₃ₐ is —(CH₂)ₐ—CH(NHR^{a})—. In some embodiments, L₃ₐ is —(CR^{1a}R^{1b})ₐ—. In some embodiments, L₃ₐ is —(CH₂)ₐ—CH(NR^{a}R^{b})—(CH₂)_{b}—.

In some embodiments of Formula (A-1), at least one A is NH and at least one A is C(O). In some embodiments of Formula (A-1), at least two A is NH and at least two A is C(O).

In some embodiments, when M is a bicyclic ring, A is a bond. In some embodiments, at least one A is a phenylene optionally substituted with one or more alkyl. In some embodiments, at least one A is thiophenylene optionally substituted with one or more alkyl. In some embodiments, at least one A is a furanylene optionally substituted with one or more alkyl. In some embodiments, at least one A is (CH₂)₀₋₄—CH═CH—(CH₂)₀₋₄, preferably —CH═CH—. In some embodiments, at least one A is —NH—N═N—. In some embodiments, at least one A is —NH—C(O)—NH—. In some embodiments, at least one A is —N(CH₃)—C₁₋₆ alkylene. In some embodiments, at least one A is

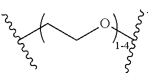

1-4 In some embodiments, at least one A is —NH—C₁₋₆ alkylene-NH—. In some embodiments, at least one A is —O—C₁₋₆ alkylene-O—.

In some embodiment, one A is 5-10 membered heteroaryl having at least one nitrogen, optionally substituted by C₁₋₆ alkyl.

In some embodiments, each M in [A-M] of Formula (A-1) is C₆₋₁₀ arylene group, 4-10 membered heterocyclene, optionally substituted 5-10 membered heteroarylene group, or C₁₋₆ alkylene; each optionally substituted by 1-3 substituents selected from H, OH, halogen, C₁₋₁₀ alkyl, NO₂, CN, NR^{a}R^{b}, C₁₋₆ haloalkyl, —C₁₋₆ alkoxyl, C₁₋₆ haloalkoxy, (C₁₋₆ alkoxy) C₁₋₆ alkyl, C₂₋₁₀alkenyl, C₂₋₁₀alkynyl, C₃₋₇ carbocyclyl, 44-10 membered heterocyclyl, C₆₋₁₀aryl, 5-10 membered heteroaryl, —(C₃₋₇carbocyclyl)C₁₋₆alkyl, (4-10 membered heterocyclyl)C₁₋₆ alkyl, (C₆₋₁₀aryl)C₁₋₆alkyl, (C₆₋₁₀aryl)C₁₋₆alkoxy, (5-10 membered heteroaryl)C₁₋₆ alkyl, —(C₃₋₇ carbocyclyl)-amine, (4-10 membered heterocyclyl)amine, (C₆₋₁₀aryl)amine, (5-10 membered heteroaryl)amine, acyl, C-carboxy, O-carboxy, C-amido, N-amido, S-sulfonamido, N-sulfonamido, —SR, COOH, or CONR^{a}R^{b}; wherein each R^{a} and R^{b} are independently H, C₁₋₁₀ alkyl, C₁₋₁₀ haloalkyl, —C₁₋₁₀ alkoxyl. In some embodiments, each M in [A-M] of Formula (A-1) is a 5-10 membered heteroarylene containing at least one heteroatoms selected from O, S, and N or a C₁₋₆ alkylene, and the heteroarylene or the a C₁₋₆ alkylene is optionally substituted with 1-3 substituents selected from OH, halogen, C₁₋₁₀ alkyl, NO₂, CN, NR^{a}R^{b}, C₁₋₆ haloalkyl, —C₁₋₆ alkoxyl, C₁₋₆haloalkoxy, C₃₋₇ carbocyclyl, 4-10 membered heterocyclyl, C₆₋₁₀aryl, 5-10 membered heteroaryl, —SR, COOH, or CONR^{a}R^{b}; wherein each R^{a} and R^{b} are independently H, C₁₋₁₀ alkyl, C₁₋₁₀ haloalkyl, —C₁₋₁₀ alkoxyl. In some embodiments, each R in [A-R] of Formula (A-1) is a 5-10 membered heteroarylene containing at least one heteroatoms selected from O, S, and N, and the heteroarylene is optionally substituted with 1-3 substituents selected from OH, C₁₋₆ alkyl, halogen, and C₁₋₆ alkoxyl.

In some embodiments of Formula (A-1), at least one M is a 5 membered heteroarylene having at least one heteroatom selected from O, N, S and optionally substituted with one or more C₁₋₁₀ alkyl. In some embodiments, at least one M is a pyrrole optionally substituted with one or more C₁₋₁₀ alkyl. In some embodiments, at least one M is a imidazole optionally substituted with one or more C₁₋₁₀ alkyl. In some embodiments of Formula (A-1), at least one M is a C₂₋₆ alkylene optionally substituted with one or more C₁₋₁₀ alkyl. In some embodiments, at least one M is a pyrrole optionally substituted with one or more C₁₋₁₀ alkyl. In some embodiments of Formula (A-1), at least one M is a bicyclic heteroarylene or arylene. In some embodiments, at least one M is a phenylene optionally substituted with one or more C₁₋₁₀ alkyl. In some embodiments, at least one M is a benzimidazole optionally substituted with one or more C₁₋₁₀ alkyl

In some embodiments, M is a 5-10 membered heteroaryl ring. In some embodiments, M is a monocyclic heteroaryl ring and at least one A adjacent to M is a bond.

In some embodiments of Formula (A-1), each E₁ independently comprises an optionally substituted thiophene-containing moiety, optionally substituted pyrrole containing moiety, optionally substituted imidazole containing moiety, or optionally substituted amine.

In some embodiments of Formula (A-1), each E₁ independently comprises a moiety selected from the group consisting of optionally substituted N-methylpyrrole, optionally substituted N-methylimidazole, optionally substituted benzimidazole moiety, and optionally substituted 3-(dimethylamino)propanimidoyl. In certain embodiments, each E₁ independently comprises thiophene, benzothiophene, C—C linked benzimidazole/thiophene-containing moiety, or C—C linked hydroxybenzimidazole/thiophene-containing moiety. In some embodiments of Formula (A-1), each E1 independently also comprises NH or CO group.

In some embodiments of Formula (A-1), each E₁ independently comprises a moiety selected from the group consisting of isophthalic acid; phthalic acid; terephthalic acid; morpholine; N,N-dimethylbenzamide; N,N-bis(trifluoromethyl)benzamide; fluorobenzene; (trifluoromethyl)benzene; nitrobenzene; phenyl acetate; phenyl 2,2,2-trifluoroacetate; phenyl dihydrogen phosphate; 2H-pyran; 2H-thiopyran; benzoic acid; isonicotinic acid; and nicotinic acid; wherein one, two, or three ring members in any of the end-group candidates can be independently substituted with C, N, S or O; and where any one, two, three, four or five of the hydrogens bound to the ring can be substituted with R^{3a}, wherein R⁵ may be independently selected from H, OH, halogen, C1-10 alkyl, NO₂, NH₂, C1-10 haloalkyl, —OC1-10 haloalkyl, COOH, and CONR^{1c}R^{1d}; wherein each R^{1c} and R^{1d} are independently H, C1-10 alkyl, C1-10 haloalkyl, or -C1-10 alkoxyl.

In some embodiments, the first terminus comprises a structure of Formula (A-2′), or a pharmaceutically acceptable salt or solvate thereof:

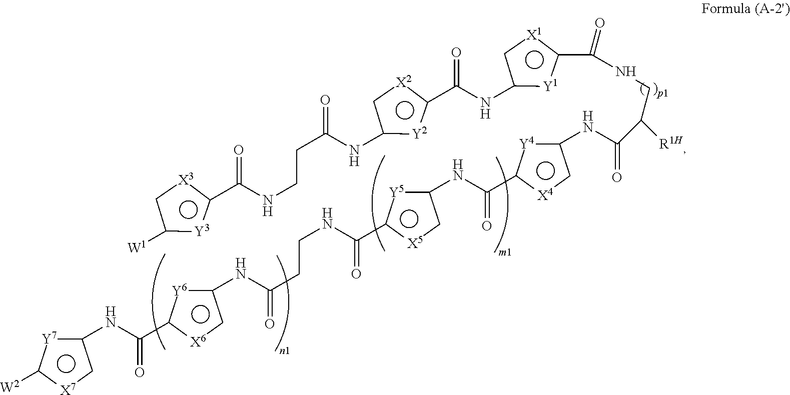

wherein:
each X¹, X², X³, X⁴, X⁵, X⁶, and X⁷ is independently O, S, or NR^{1D};
each Y¹, Y², Y³, Y⁴, Y⁵, Y⁶, and Y⁷ is independently CH or N;
W¹ is hydrogen, optionally substituted 5-10 membered heteroaryl, C₁-C₆ alkyl, —C(O)—NR^{1E}R^{1F}, —NR^{1E}—C(O)—NR^{1E}R^{1F};
W² is hydrogen, optionally substituted 5-10 membered heteroaryl, C₁-C₆ alkyl, or —C(O)—NR^{1E}R^{1F};
m¹ is 0, 1, 2, or 3;
n¹ is 0, 1, 2, or 3;
p¹ is 1, 2, 3, or 4.
each R^{1D} and R^{1E} is independently hydrogen, or optionally substituted C₁-C₆ alkyl;
R^{1F} is hydrogen, an optionally substituted C₁-C₁₀ alkyl, C₁-C₁₀ heteroalkyl, PEG₁₋₂₀, or one or more AA, wherein AA is one or more amino acids selected from β-alanine, lysine, and arginine; and
R^{1H} is hydrogen, amino, cyano, or optionally substituted C₁-C₁₀ alkyl, C₁-C₁₀ heteroalkyl.

In some embodiments, the first terminus is connected to a linker through W¹.

In some embodiments, W² is —C(O)—NR^{1E}R^{1F}. In some embodiments, W² is —C(O)NH₂. In some embodiments, W² is —C(O)-β-alanine.

In some embodiments, W² provides for a site of attachment to a linker moiety. In some embodiments, W² is —C(O)NH—(CH₂)₂—C(O)—**, wherein the linker moiety is attached at **. In some embodiments, W² is —C(O)—NH—**, wherein the linker moiety is attached at **. In some embodiments, W² is —C(O)—**, wherein the linker moiety is attached at **.

In some embodiments, the first terminus comprises a structure of Formula (A-2), or a pharmaceutically acceptable salt or solvate thereof:

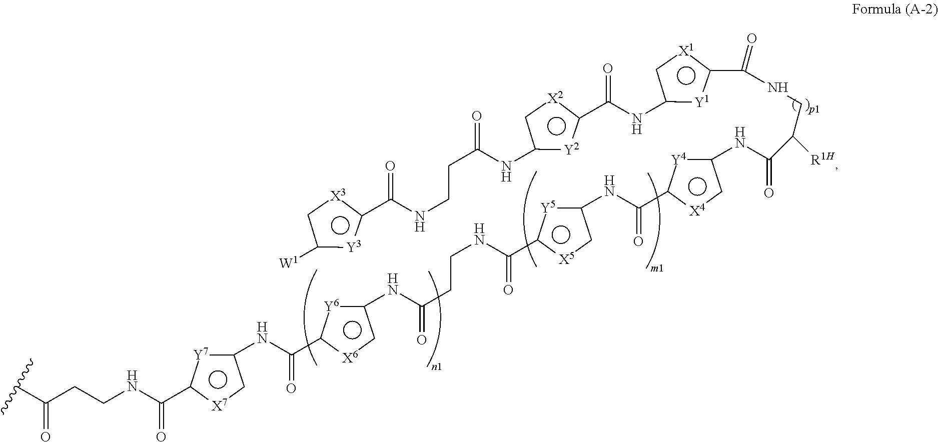

wherein:
each X¹, X², X³, X⁴, X⁵, X⁶, and X⁷ is independently O, S, or NR^{1D};
each Y¹, Y², Y³, Y⁴, Y⁵, Y⁶, and Y⁷ is independently CH or N;
W¹ is hydrogen, optionally substituted 5-10 membered heteroaryl, C₁-C₆ alkyl, —C(O)—NR^{1E}R^{1F}, —NR^{1E}—C(O)—NR^{1E}R^{1F};
m¹ is 0, 1, 2, or 3;
n¹ is 0, 1, 2, or 3;
p¹ is 1, 2, 3, or 4.
each R^{1D} and R^{1E} is independently hydrogen or optionally substituted C₁-C₆ alkyl; and
R^{1F} is hydrogen, an optionally substituted C₁-C₁₀ alkyl, C₁-C₁₀ heteroalkyl, PEG₁₋₂₀, or one or more AA, wherein AA is one or more amino acids selected from β-alanine, lysine, and arginine.

In some embodiments, each X¹, X², X³, X⁴, X⁵, X⁶, and X⁷ is independently —NR^{1D}, wherein R^{1D} is C₁-C₆ alkyl. In some embodiments, each X¹, X², X³, X⁴, X⁵, X⁶, and X⁷ is independently —NCH₃. In some embodiments, R^{1D} is a branched or straight chain C₁-C₆ alkyl.

In some embodiments, m¹ is 0 or 1 and n¹ is 0 or 1.

In some embodiments, p¹ is 2 or 3.

In some embodiments, W¹ is hydrogen.

In some embodiments, W¹ is —C(O)—NR^{1E}R^{1F}, wherein R^{1D} and R^{1E} is independently hydrogen or C₁-C₆ alkyl or an optionally substituted 5-10 membered heteroaryl. In some embodiment, W¹ is —C(O)-pyrazole or —C(O)-imidazole.

In some embodiments, W¹ is

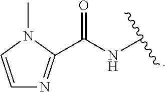 [SMILES: *NC(=O)c1nccn1C]

In some embodiments, W¹ is

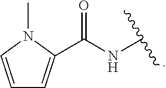 [SMILES: *NC(=O)c1cccn1C]

In some embodiments, W¹ is hydrogen.

In some embodiments, the first terminus comprises the structure of Formula (A-3), or a pharmaceutically acceptable salt or solvate thereof:

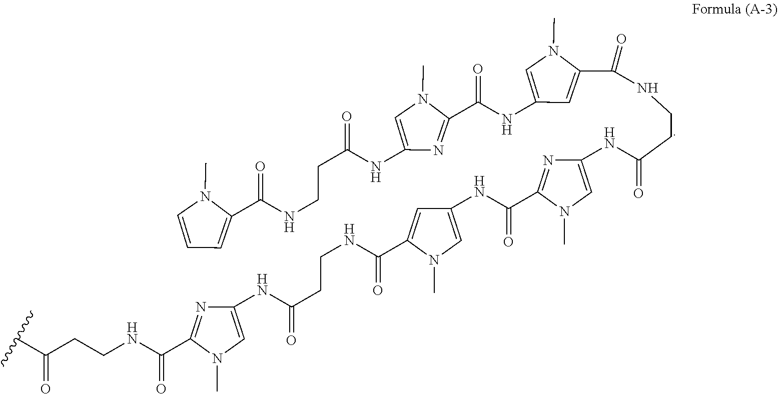 [SMILES: *C(=O)CCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C]

In some embodiments, the first terminus comprises the structure of Formula (A-4), or a pharmaceutically acceptable salt or solvate thereof:

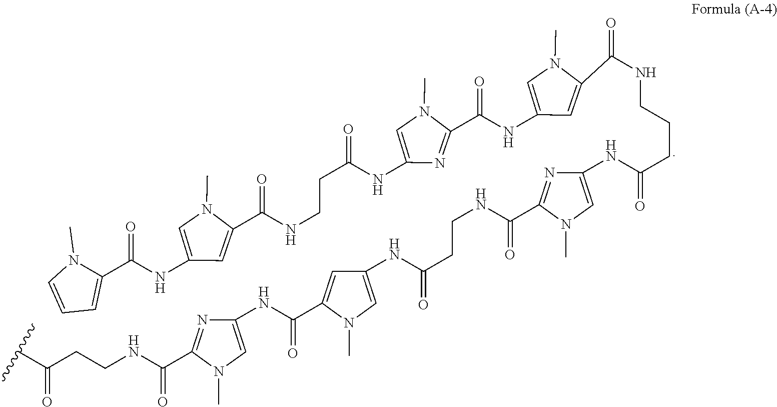 [SMILES: *C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7cccn7C)cn6C)cn5C)cn4C)cn3C)cn2C)cn1C]

In some embodiments, the first terminus comprises the structure of Formula (A-5), or a pharmaceutically acceptable salt or solvate thereof:

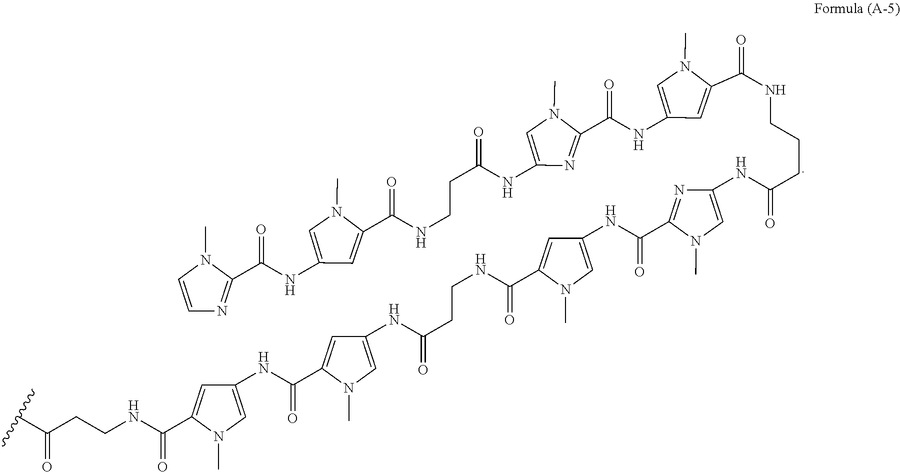 [SMILES: *C(=O)CCNC(=O)c1cc(NC(=O)c2cc(NC(=O)CCNC(=O)c3cc(NC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cn1C]

In some embodiments, the first terminus is not:

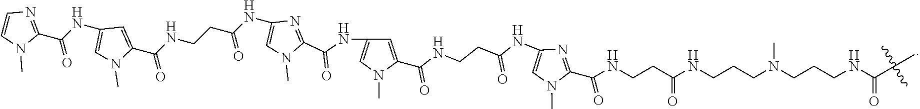 [SMILES: *C(=O)NCCCN(C)CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C]

The first terminus in the molecules described herein has a high binding affinity to a sequence having multiple repeats of CAG or CTG and binds to the target nucleotide repeats preferentially over other nucleotide repeats or other nucleotide sequences. In some embodiments, the first terminus has a higher binding affinity to a sequence having multiple repeats of CAG or CTG than to a sequence having repeats of CGG. In some embodiments, the first terminus has a higher binding affinity to a sequence having multiple repeats of CAG or CTG than to a sequence having repeats of CCG. In some embodiments, the first terminus has a higher binding affinity to a sequence having multiple repeats of CAG or CTG than to a sequence having repeats of CCTG. In some embodiments, the first terminus has a higher binding affinity to a sequence having multiple repeats of CAG or CTG than to a sequence having repeats of TGGAA. In some embodiments, the first terminus has a higher binding affinity to a sequence having multiple repeats of CAG or CTG than to a sequence having repeats of GGGGCC. In some embodiments, the first terminus has a higher binding affinity to a sequence having multiple repeats of CAG or CTG than to a sequence having repeats of GAA.

Due to the preferential binding between the first terminus and the target nucleotide repeat, the transcription modulation molecules described herein become localized around regions having multiple repeats of CAG or CTG. In some embodiments, the local concentration of the first terminus or the molecules described herein is higher near a sequence having multiple repeats of CAG or CTG than near a sequence having repeats of CGG. In some embodiments, the local concentration of the first terminus or the molecules described herein is higher near a sequence having multiple repeats of CAG or CTG than near a sequence having repeats of CCG. In some embodiments, the local concentration of the first terminus or the molecules described herein is higher near a sequence having multiple repeats of CAG or CTG than near a sequence having repeats of CCTG. In some embodiments, the local concentration of the first terminus or the molecules described herein is higher near a sequence having multiple repeats of CAG or CTG than near a sequence having repeats of TGGAA. In some embodiments, the local concentration of the first terminus or the molecules described herein is higher near a sequence having multiple repeats of CAG or CTG than near a sequence having repeats of GGGGCC. In some embodiments, the local concentration of the first terminus or the molecules described herein is higher near a sequence having multiple repeats of CAG or CTG than near a sequence having repeats of GAA.

In one aspect, the molecules of the present disclosure preferentially bind to the repeated CAG or CTG of dmpk, atxn1, atxn2, atxn3, cacna1a, atxn7, atxn8, atxn80s, ttbk2, tcf4, ppp2r2b, tbp, htt, jph3, ar, or atn1 than to CAG or CTG elsewhere in the subject's DNA, due to the high number of CAG or CTG repeats associated with dmpk, atxn1, atxn2, atxn3, cacna1a, atxn7, atxn8, atxn80s, ttbk2, tcf4, ppp2r2b, tbp, htt, jph3, ar, or atn1. In one aspect, the molecules of the present disclosure are more likely to bind to the repeated CTG of dmpk than to CTG elsewhere in the subject's DNA due to the high number of CTG repeats associated with dmpk. In one aspect, the molecules of the present disclosure are more likely to bind to the repeated CAG of atxn1, atxn2, atxn3, cacna1a, atxn7, ppp2r2b, tbp, htt, jph3, ar, or atn1 than to CAG elsewhere in the subject's DNA, due to the high number of CAG repeats associated with atxn1, atxn2, atxn3, cacna1a, atxn7, ppp2r2b, tbp, htt, jph3, ar, or atn1. In one aspect, the molecules of the present disclosure are more likely to bind to the repeated CTG of atxn8 or atxn80s than to CTG elsewhere in the subject's DNA due to the high number of CTG repeats associated with atxn8 or atxn80s. In one aspect, the molecules of the present disclosure are more likely to bind to the repeated CAG of TCF4 gene than to CAG elsewhere in the subject's DNA, due to the high number of CAG repeats associated with TCF4. In one aspect, the molecules of the present disclosure are more likely to bind to the repeated CAG of TTBK2 gene than to CAG elsewhere in the subject's DNA, due to the high number of CAG repeats associated with TTBK2.

The first terminus is localized to a sequence having multiple repeats of CAG or CTG and binds to the target nucleotide repeats preferentially over other nucleotide repeats. In some embodiments, the sequence has at least 2, 3, 4, 5, 8, 10, 12, 15, 20, 25, 30, 40, 50, 100, 200, 300, 400, or 500 repeats of CAG or CTG. In certain embodiments, the sequence comprises at least 1000 nucleotide repeats of CAG or CTG. In certain embodiments, the sequence comprises at least 500 nucleotide repeats of CAG or CTG. In certain embodiments, the sequence comprises at least 200 nucleotide repeats of CAG or CTG. In certain embodiments, the sequence comprises at least 100 nucleotide repeats of CAG or CTG. In certain embodiments, the sequence comprises at least 50 nucleotide repeats of CAG or CTG. In certain embodiments, the sequence comprises at least 20 nucleotide repeats of CAG or CTG.

The polyamide composed of a pre-selected combination of subunits can selectively bind to the DNA in the minor groove. In their hairpin structure, antiparallel side-by-side pairings of two aromatic amino acids bind to DNA sequences, with a polyamide ring packed specifically against each DNA base. N-Methylpyrrole (Py) favors T, A, and C bases, excluding G; N-methylimidazole (Im) is a G-reader; and 3-hydroxyl-N-methylpyrrol (Hp) is specific for thymine base. The nucleotide base pairs can be recognized using different pairings of the amino acid subunits using the paring principle shown in Table 1A and 1B below. For example, an Im/Py pairing reads G·C by symmetry, a Py/Im pairing reads C·G, an Hp/Py pairing can distinguish T·A from A·T, G·C, and C·G, and a Py/Py pairing nonspecifically discriminates both A·T and T·A from G·C and C·G.

In some embodiments, the first terminus comprises Im corresponding to the nucleotide G; Im or Nt corresponding to the nucleotide pair G; Py corresponding to the nucleotide C, wherein Im is N-alkyl imidazole, Py is N-alkyl pyrrole, Hp is 3-hydroxy N-methyl pyrrole, and β-alanine. In some embodiments, the first terminus comprises Im/Py to correspond to the nucleotide pair G/C, Py/Im to correspond to the nucleotide pair C/G, and wherein Im is N-alkyl imidazole (e.g, N-methyl imidazole), Py is N-alkyl pyrrole (e.g., N-methyl pyrrole), and Hp is 3-hydroxy N-methyl pyrrole.

TABLE 1A
Base pairing for single amino acid subunit (Favored (+), disfavored (−)

| Subunit | G | C | A | T |
|---|---|---|---|---|
| Py | − | + | + | + |
| Im | + | − | − | |
| 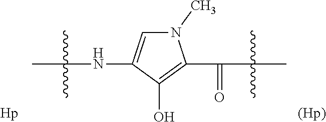 [SMILES: *Nc1cn(C)c(C(*)=O)c1O] | − | − | − | + |
| 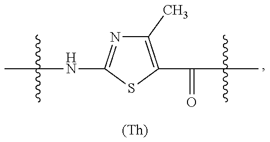 | − | − | + | + |
| 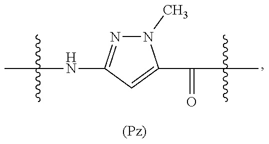 [SMILES: *Nc1cc(C(*)=O)n(C)n1] | − | − | + | + |
| 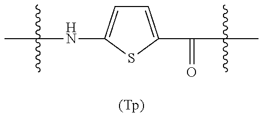 [SMILES: *Nc1ccc(C(*)=O)s1] | − | − | + | + |
| 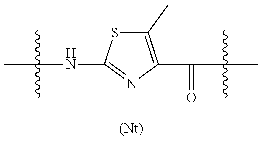 [SMILES: *Nc1nc(C(*)=O)c(C)s1] | + | − | − | − |
| 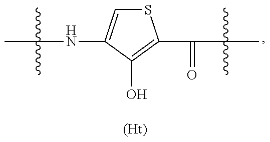 [SMILES: *Nc1csc(C(*)=O)c1O] | − | − | − | + |
| 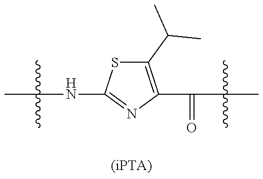 [SMILES: *Nc1nc(C(*)=O)c(C(C)C)s1] | + | − | − | − |
| 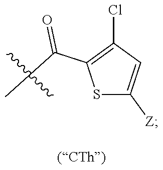 | − | − | − | + |
| 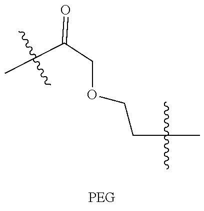 [SMILES: *CCOCC(*)=O] | − | + | + | + |
| 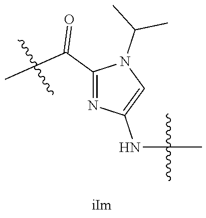 [SMILES: *Nc1cn(C(C)C)c(C(*)=O)n1] | + | − | − | − |
| 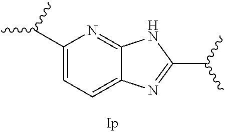 [SMILES: *c1ccc2nc(*)[nH]c2n1] | + | − | − | − |
| 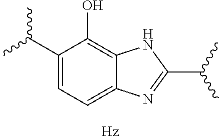 [SMILES: *c1nc2ccc(*)c(O)c2[nH]1] | − | − | − | + |
| 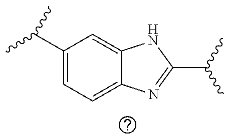 [SMILES: *c1ccc2nc(*)[nH]c2c1] | − | − | − | + |
| 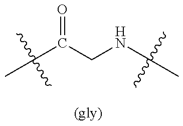 [SMILES: *NCC(*)=O] | − | − | − | − |
| 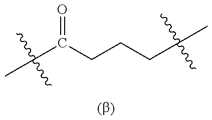 [SMILES: *CCCC(*)=O] | − | − | + | + |
| 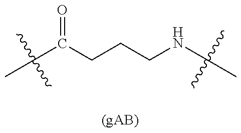 [SMILES: *NCCCC(*)=O] | − | − | + (as a part of the turn) | + (as a part of the turn) |
| 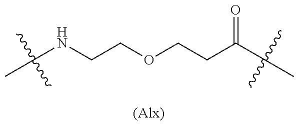 [SMILES: *NCCOCCC(*)=O] | − | + | − | − |
| 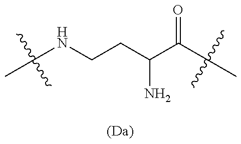 [SMILES: *NCCC(N)C(*)=O] | − | − | + | + |
| 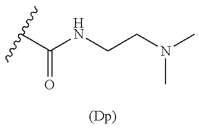 [SMILES: *C(=O)NCCN(C)C] | − | − | + | + |
| 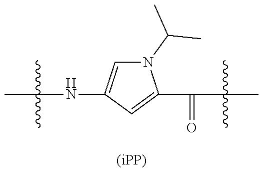 [SMILES: *Nc1cc(C(*)=O)n(C(C)C)c1] | − | − | + | + |
| 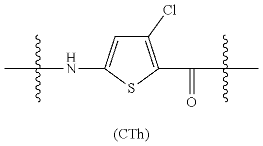 [SMILES: *Nc1cc(Cl)c(C(*)=O)s1 ; "(CTh)"] | + | + | − | − |
| 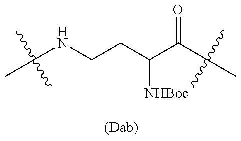 [SMILES: *NCCC(NC(=O)OC(C)(C)C)C(*)=O] | − | − | + | + |
| 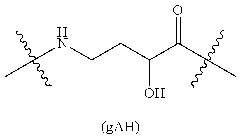 [SMILES: *NCCC(O)C(*)=O] | − | − | + | + |

| | |
|---|---|
| 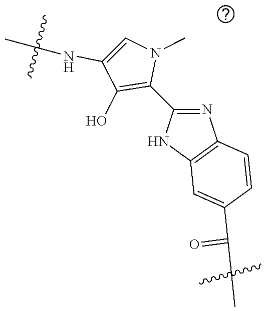 [SMILES: *Nc1cn(C)c(-c2nc3ccc(C(*)=O)cc3[nH]2)c1O] | WW* (bind to two nucleotides with same selectivity as Hp-Py) |
| 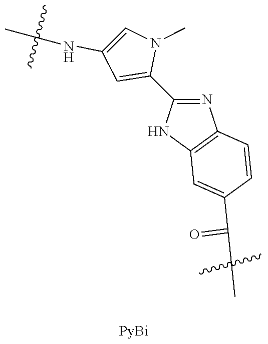 [SMILES: *Nc1cc(-c2nc3ccc(C(*)=O)cc3[nH]2)n(C)c1] | WW* (bind to two nucleotides with same selectivity as Py-Py) |
| 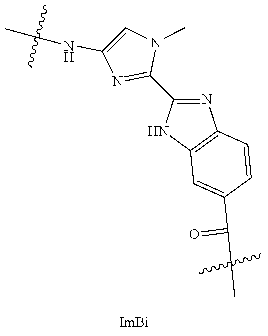 [SMILES: *Nc1cn(C)c(-c2nc3ccc(C(*)=O)cc3[nH]2)n1] | GW* (bind to two nucleotides with same selectivity as Im-Py) |

*The subunit HpBi, ImBi, and PyBi function as a conjugate of two monomer subunits and bind to two nucleotides. The binding property of HpBi, ImBi, and PyBi corresponds to Hp-Py, Im-Py, and Py-Py respectively.

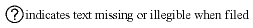

TABLE 1B
Base pairing for hairpin polyamide

| | G · C | C · G | T · A | A · T |
|---|---|---|---|---|
| Im/β | + | − | − | − |
| β/Im | − | + | − | − |
| Py/β | − | − | + | + |
| β/Py | − | − | + | + |
| β/β | − | − | + | + |
| Py/Py | − | − | + | + |
| Im/Im | − | − | − | − |
| Im/Py | + | − | − | − |
| Py/Im | − | − | − | − |
| Th/Py | − | − | + | − |
| Py/Th | − | − | − | + |
| Th/Im | + | − | − | − |
| Im/Th | − | + | − | − |
| β/Th | − | − | + | − |
| Th/β | − | − | − | + |
| Hp/Py, | − | − | + | − |
| Py/Hp, | − | − | − | + |
| Hp/Im | + | − | − | − |
| Im/Hp | − | + | − | − |
| Tn/Py | − | − | + | + |
| Py/Tn, | − | − | + | + |
| Ht/Py, | − | − | + | + |
| Py/Ht, | − | − | + | + |
| Bi/Py, | − | − | + | + |
| Py/Bi, | − | − | + | + |
| β/Bi | − | − | + | + |
| Bi/β | − | − | + | + |
| Bi/Im, | − | − | − | − |
| Im/Bi, | + | − | − | − |
| Tp/Py, | − | − | + | + |
| Py/Tp, | − | − | + | + |
| β/Tp | − | − | + | + |
| Tp/β | − | − | + | + |
| Tp/Im, | − | + | − | − |
| Im/Tp | + | − | − | − |
| Tp/Tp | − | − | + | + |
| Tp/Tn | − | − | + | + |
| Tn/Tp | − | − | + | + |
| Hz/Py, | − | − | + | − |
| Py/Hz, | − | − | − | + |
| Ip/Py | + | − | − | − |
| Py/Ip, | − | + | − | − |
| Bi/Hz, | − | − | + | + |
| Hz/Bi, | − | − | + | + |
| Bi/Bi | − | + | + | − |
| Th/Py, | − | − | + | + |
| Py/Th | − | − | + | + |
| Im/gAB | + | − | − | − |
| gAB/Im | − | + | − | − |
| Py/gAB | + | − | − | − |
| gAB/Py | − | + | − | − |
| gAB/β | − | − | + | + |
| β/gAB | − | − | + | + |
| Im/Dp | + | − | − | − |
| Dp/Im | − | + | − | − |
| Py/Dp | − | − | + | + |
| Dp/Py | − | − | + | + |
| Dp/β | − | − | + | + |

Each of HpBi, ImBi, and PyBi can bind to two nucleotides and have binding properties corresponding to Hp-Py, Im-Py, and Py-Py respectively. HpBi, ImBi, and PyBi can be paired with two monomer subunits or with themselves in a hairpin structure to bind to two nucleotide pairs.

The monomer subunits of the polyamide can be strung together based on the paring principles shown in Table 1A and Table 1B. The monomer subunits of the polyamide can be strung together based on the paring principles shown in Table 1C and Table 1D.

Table 1C shows an example of the monomer subunits that can bind to the specific nucleotide. The first terminus can include a polyamide described having several monomer subunits stung together, with a monomer subunit selected from each row. For example, the polyamide can include Py-Py-Im that binds to CAG, with Py is selected from the C column, Py is selected from the A column, and Im selected from the first G column. The polyamide can be any combinations of the subunits of CAGCAG, with a subunit selected from each column in Table 1C, wherein the subunits are strung together following the CAG binding order. In another example, the polyamide can include Py-3-Im that binds to CTG, with Py selected from the C column, (3 from the T column, and Im from the G column.

In addition, the polyamide can also include a partial or multiple sets of the five subunits, such as 1.5, 2, 2.5, 3, 3.5, or 4 sets of the three subunits. The polyamide can include 3, 4, 5, 6, 7, 8, 9, 10, 12, 14, and 16 monomer subunits. The multiple sets can be joined together by W. In addition to the five subunits or ten subunits, the polyamide can also include 1-4 additional subunits that can link multiple sets of the five subunits.

The polyamide can include monomer subunits that bind to 2, 3, 4, or 5 nucleotides of CAG or CTG. For example, the polyamide can bind to CA, CAG, AGC, CAGC, CAGCA, CAGCAG. The polyamide can include monomer subunits that bind to 3, 4, 5, 6, 7, 8, 9, or 10 nucleotides of CAG repeat. For example, the polyamide can bind to CT, CTG, TGC, CTGC, CTGCT, CTGCTG, CTGCTGC. The polyamide can include monomer subunits that bind to 3, 4, 5, 6, 7, 8, 9, or 10 nucleotides of CTG repeat. The nucleotides can be joined by W.

The monomer subunit, when positioned as a terminal unit, does not have an amine, carbonyl, or a carboxylic acid group at the terminal. The amine or carboxylic acid group in the terminal is replaced by a hydrogen. For example, Py, when used as a terminal unit, is understood to have the structure of

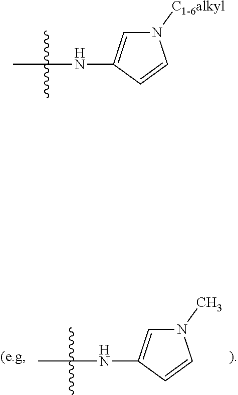

and Im, when positioned as a terminal unit, is understood to have the structure of

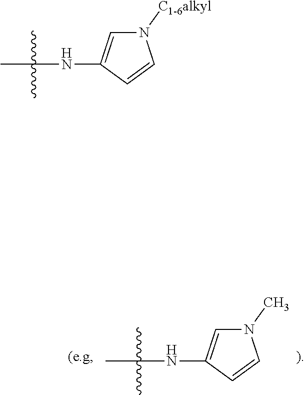

In addition, when Py or Im is used as a terminal unit, Py and Im can be respectively replaced by PyT

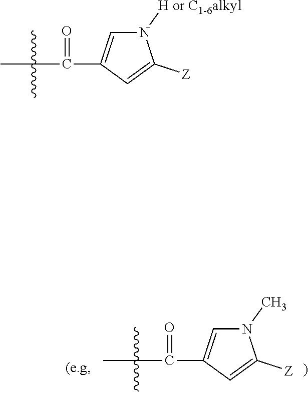

### and ImT

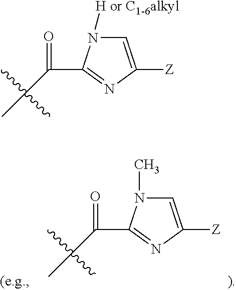

The linear polyamide can have nonlimiting examples including but not limited to Py-Py-Im-Py-Py-Im-Py-Py-Im, β-Im-Py-β-Im-Py-β-Im, Im-Py-Py-Im-Py-Py-Im, Im-Py-Py-Im-Py-β, β-Im-Py-Py-Im-Py-β, Py-Py-Im-β-β-Im-Py-Py-Im, and any combinations thereof.

TABLE 1C
Examples of monomer subunits in a linear polyamide that binds to CAG or CTG.

| | | | | | | | |
|---|---|---|---|---|---|---|---|
| Nucleotide | C | A | G | or | C | T | G |

| | | | | | | |
|---|---|---|---|---|---|---|
| Subunit that | Py or PyT | Py | Im or ImT | Py or PyT | β | Im or ImT |
| selectively | iIm or iImT | Th | iIm or iImT | ilm or ilmT | Py | ilm or ilmT |
| binds to | PEG | Pz | PEG | PEG | Hp | PEG |
| nucleotide | CTh | Tp | CTh | CTh | Th | CTh |
| | Alx | PEG | Nt | Alx | Pz | Nt |
| | | β | iPTA | | Tp | iPTA |
| | | iPP | Ip | | Ht | Ip |
| | | Da | CTh | | CTh | CTh |
| | | Dp | | | PEG | |
| | | Dab | | | Hz | |
| | | gAH | | | Bi | |
| | | | | | Da | |
| | | | | | Dp | |
| | | | | | iPP | |
| | | | | | Dab | |
| | | | | | gAH | |

The DNA-binding moiety can also include a hairpin polyamide having subunits that are strung together based on the pairing principle shown in Table 1B. Table 1D shows some examples of the monomer subunit pairs that selectively bind to the nucleotide pair. The hairpin polyamide can include 2n monomer subunits (n is an integer in the range of 2-8), and the polyamide also includes a W in the center of the monomer subunits. W can be —(CH₂)ₐ—NR¹—(CH₂)_{b}—, —(CH₂)ₐ—, —(CH₂)ₐ—O—(CH₂)_{b}—, —(CH₂)ₐ—CH(NHR¹)—, —(CH₂)ₐ—CH(NHR¹)—, —(CR²R³)ₐ— or —(CH₂)ₐ—CH(NR¹₃)⁺—(CH₂)_{b}—, wherein each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, W is —(CH₂)—CH(NH₃)⁺—(CH₂)— or —(CH₂)—CH₂CH(NH₃)⁺—. In some embodiments, R¹ is H. In some embodiments, R¹ is C₁₋₆ alkyl optionally substituted by 1-3 substituents selected from —C(O)-phenyl. In some embodiments, W is —(CR²R³)—(CH₂)ₐ— or —(CH₂)ₐ—(CR²R³)—(CH₂)_{b}—, wherein each a is independently 1-3, b is 0-3, and each R² and R³ are independently H, halogen, OH, NHAc, or C₁₋₄ alky. W can be an aliphatic amino acid residue shown in Table 4 such as gAB. W is gAB, it favors binding to T.

Because the target gene can include multiple repeats of CAG or CTG, the subunits can be strung together to bind at least two, three, four, five, six, seven, eight, nine, or ten nucleotides in one or more CAG or CTG repeat (e.g., CAGCAG, or CTGCTGCTG). For example, the polyamide can bind to the CAG or CTG repeat by binding to a partial copy, a full copy, or a multiple repeats of CAG or CTG such as CA, CAG, AGC, CAGC, CAGCA, CAGCAG, CT, CTG, TGC, CTGC, CTGCT, CTGCTG. For example, the polyamide can include Im-Im-Im-Im-β-β-W-Im-Im-β-Py-β-Py that binds to GGGGCC and its complementary nucleotides on a double strand DNA, in which the Im/Py pair binds to the G·C, the Im/β pair binds to G·C, the Im/Py pair binds to G·C, the Im/β binds to G·C, and β/Im binds to C·G; and β/Im binds to C·G. In one example Py-β-Im-β-W-Im-Py-Py-Im that binds to CTGC and its complementary nucleotides on a double strand DNA, in which Py/Im pair binds to C·G, β/Py pair binds to T·A, Im/Im pair binds to C·G, and β/Py pair binds to C·G. W can be an aliphatic amino acid residue such as gAB or other appropriate spacers as shown in Table 4. In another example, the polyamide can include Im-Py-Py-Im-Py-gAB-Im-Py-Py-Im-β that binds to GCTGC and its complementary nucleotides on a double strand DNA, in which the Im/β pair binds to G·C, the Py/Im pair binds to C·G, the Py/Py binds to T·A, Im/Py pair binds to the G·C, and Py/Im binds to C·G. In another example, Im-Py-Py-Im-Py-gAB-Im-Py-Py binds to GCTGC with a part of the complementary nucleotides (ACG) on the double strand DNA, in which Im binds to G, Py binds to C, Py/Py binds to T·A, Im/Py binds to the G·C, and Py/Im binds to the C·G.

Some additional examples of the polyamide include but are not limited to Py-β-Im-β-gAB-Im-Py-Py-Im, Im-Py-Py-Im-Py-gAB-Im-Py-Py-Im-β, Py-Py-Im-Py-gAB-Im-Py-Py-Im-β, Py-Py-Im-Py-gAB-Im-Py-Py-Im, Py-Py-Im-Py-gAB-Im-Py-Py, Im-Py-Py-Im-Py-gAB-Im-Py-Py-Im, Im-Py-Py-Im-Py-gAB-Im-Py-Py, and any combinations thereof.

TABLE 1D
Examples of monomer pairs in a hairpin or
H-pin polyamide that binds to CAG or CTG.

| | | | | | | | |
|---|---|---|---|---|---|---|---|
| Nucleotide | C•G | A•T | G•C | or | C•G | T•A | G•C |

| | | | | | | |
|---|---|---|---|---|---|---|
| Subunit pairs | β/Im | Py/β | Im/β | β/Im | Py/β | Im/β |
| that selectively | Py/Im | β/Py | Im/Py | Py/Im | β/Py | Im/Py |
| binds to | Im/Th | β/β | Th/Im | Im/Th | β/β | Th/Im |
| nucleotide | Im/Hp | Py/Py | Hp/Im | Im/Hp | Py/Py | Hp/Im |
| | Bi/Im | Py/Th | Im/Bi | Bi/Im | Th/Py | Im/Bi |
| | Tp/Im | Th/β | Im/Tp | Tp/Im | β/Th | Im/Tp |
| | Py/Ip | Py/Hp, | Ip/Py | Py/Ip | Hp/Py | Ip/Py |
| | Bi/Bi | Tn/Py | Im/gAB | Bi/Bi | Bi/Py | Im/gAB |
| | gAB/Im | Py/Tn, | Py/gAB | gAB/Im | Py/Bi | Py/gAB |
| | gAB/Py | Ht/Py, | Im/Dp | gAB/Py | β/Bi | Im/Dp |
| | Dp/Im | Py/Ht, | | Dp/Im | Bi/β | |
| | | Bi/Py, | | | Tp/Py | |
| | | Py/Bi, | | | Py/Tp | |
| | | β/Bi | | | β/Tp | |
| | | Bi/β | | | Tp/β | |
| | | Tp/Py, | | | Tp/Tp | |
| | | Py/Tp, | | | Tp/Tn | |
| | | β/Tp | | | Tn/Tp | |
| | | Tp/β | | | Hz/Py | |
| | | Tp/Tp | | | Bi/Hz, | |
| | | Tp/Tn | | | Hz/Bi, | |
| | | Tn/Tp | | | Bi/Bi | |
| | | Py/Hz, | | | Th/Py, | |
| | | Bi/Hz, | | | Py/Th | |
| | | Hz/Bi, | | | gAB/β | |
| | | Bi/Bi | | | β/gAB | |
| | | Th/Py, | | | Py/Dp | |
| | | Py/Th | | | Dp/Py | |
| | | gAB/β | | | Dp/β | |
| | | β/gAB | | | β/Dp | |
| | | Py/Dp | | | | |
| | | Dp/Py | | | | |
| | | Dp/β | | | | |

Recognition of a nucleotide repeat or DNA sequence by two antiparallel polyamide strands depends on a code of side-by-side aromatic amino acid pairs in the minor groove, usually oriented N to C with respect to the 5′ to 3′ direction of the DNA helix. Enhanced affinity and specificity of polyamide nucleotide binding is accomplished by covalently linking the antiparallel strands. The “hairpin motif” connects the N and C termini of the two strands with a W (e.g., gamma-aminobutyric acid unit (gamma-turn)) to form a folded linear chain. The “H-pin motif” connects the antiparallel strands across a central or near central ring/ring pairs by a short, flexible bridge.

The DNA-binding moiety can also include a H-pin polyamide having subunits that are strung together based on the pairing principles shown in Table 1A and/or Table 1B. Table 1C shows some examples of the monomer subunit that selectively binds to the nucleotide, and Table 1D shows some examples of the monomer subunit pairs that selectively bind to the nucleotide pair. The h-pin polyamide can include 2 strands and each strand can have a number of monomer subunits (each strand can include 2-8 monomer subunits), and the polyamide also includes a bridge L₁ to connect the two strands in the center or near the center of each strand. At least one or two of the monomer subunits on each strand are paired with the corresponding monomer subunits on the other stand following the paring principle in Table 1D to favor binding of either G·C or C·G pair, and these monomer subunit pairs are often positioned in the center, close to center region, at or close to the bridge that connects the two strands. In some instances, the H-pin polyamide can have all of the monomer subunits be paired with the corresponding monomer subunits on the antiparallel strand based on the paring principle in Table 1B and 1D to bind to the nucleotide pairs on the double strand DNA. In some instances, the H-pin polyamide can have a part of the monomer subunits (2, 3, 4, 5, or 6) be paired with the corresponding monomer subunits on the antiparallel strand based on the binding principle in Table 1B and 1D to bind to the nucleotide pairs on the double strand DNA, while the rest of the monomer subunit binds to the nucleotide based on the binding principle in Table 1A and 1C but does not pair with the monomer subunit on the antiparallel strand. The h-pin polyamide can have one or more overhanging monomer subunit that binds to the nucleotide but does not pair with the monomer subunit on the antiparallel strand.

Another polyamide structure that derives from the h-pin structure is to connect the two antiparallel strands at the end through a bridge, while only the two monomer subunits that are connected by the bridge form a pair that bind to the nucleotide pair G·C or C·G based on the binding principle in Table 1B/1D, but the rest of the monomer subunits on the strand form an overhang, bind to the nucleotide based on the binding principle in Table 1A and/or 1C and do not pair with the monomer subunit on the other strand.

The bridge can be is a bivalent or trivalent group selected from

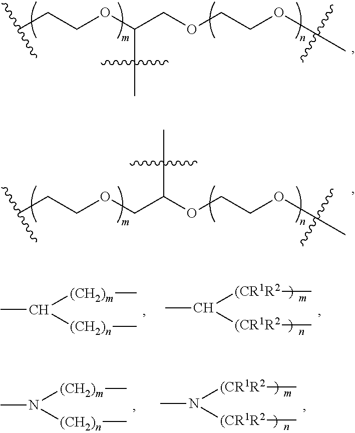

a C₁₋₁₀ alkylene, —NH—C₀₋₆ alkylene-C(O)—, —N(CH₃)—C₀₋₆ alkylene, and

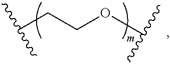

—(CH₂)ₐ—NR¹—(CH₂)_{b}—, —(CH₂)ₐ—, —(CH₂)ₐ—O—(CH₂)_{b}—, —(CH₂)ₐ—CH(NHR¹)—, —(CH₂)ₐ—CH(NHR¹)—, —(CR²R³)ₐ— or —(CH₂)ₐ—CH(NR¹₃)⁺—(CH₂)_{b}—, wherein m is an integer in the range of 0 to 10; n is an integer in the range of 0 to 10; each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, W is —(CH₂)—CH(NH₃)⁺—(CH₂)— or —(CH₂)—CH₂CH(NH₃)⁺—. In some embodiments, R¹ is H. In some embodiments, R¹ is C₁₋₆ alkyl optionally substituted by 1-3 substituents selected from —C(O)-phenyl. In some embodiments, L₁ is —(CR²R³)—(CH₂)ₐ— or —(CH₂)ₐ—(CR²R³)—(CH₂)_{b}—, wherein each a is independently 1-3, b is 0-3, and each R² and R³ are independently H, halogen, OH, NHAc, or C₁₋₄ alky. L₁ can be a C₂₋₉ alkylene or (PEG)₂₋₈.

Some additional examples of the polyamide include but are not limited to Py-Py-Im-Py (linked in the middle—either position 2 or 3) to Im-Py-Py-Im, Py-β-Im-β (linked in the middle—either position 2 or 3) Im-Py-Py-Im, Im-Py-Py-Im-Py (linked in the middle—either position 2, 3, or 4) Im-Py-Py-Im-β, Py-Py-Im-Py (middle position 2 or 3 of Py-Py-Im-Py linked with position 2, 3, or 4 of Im-Py-Py-Im-β) Im-Py-Py-Im-β, Py-Py-Im-Py (linked in the middle—either position 2 or 3) Im-Py-Py-Im, Py-Py-Im-Py (middle position 2 or 3 of Py-Py-Im-Py linked with position 2 of Im-Py-Py) Im-Py-Py, Im-Py-Py-Im-Py (either middle position 2, 3, or 4 of Im-Py-Py-Im-Py linked with middle position 2 or 3 of Im-Py-Py-Im) Im-Py-Py-Im, Im-Py-Py-Im-Py (middle position 2, 3, or 4 linked with middle position of Im-Py-Py) Im-Py-Py.

### Second Terminus—Regulatory Binding Moiety

In certain embodiments, the regulatory molecule is chosen from a nucleosome remodeling factor (NURF), a bromodomain PHD finger transcription factor (BPTF), a ten-eleven translocation enzyme (TET), methylcytosine dioxygenase (TET1), a DNA demethylase, a helicase, an acetyltransferase, and a histone deacetylase (“HDAC”). In certain embodiments, the regulatory molecule is selected from CDK9i, CDK7i, CDK12/13i, Pan-CDKi, a L3MBTL3 recruiter, a CBX recruiter, or an EED recruiter.

The binding affinity between the regulatory protein and the second terminus can be adjusted based on the composition of the molecule or type of protein. In some embodiments, the second terminus binds the regulatory molecule with an affinity of less than about 600 nM, about 500 nM, about 400 nM, about 300 nM, about 250 nM, about 200 nM, about 150 nM, about 100 nM, or about 50 nM. In some embodiments, the second terminus binds the regulatory molecule with an affinity of less than about 300 nM. In some embodiments, the second terminus binds the regulatory molecule with an affinity of less than about 200 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, 100-300 nM, or 50-200 nM.

In some embodiments, the protein-binding moiety binds to the regulatory molecule that is selected from the group consisting of a CREB binding protein (CBP), a P300, an O-linked β-N-acetylglucosamine-transferase-(OGT-), a P300-CBP-associated-factor- (PCAF-), histone methyltransferase, histone demethylase, chromodomain, a cyclin-dependent-kinase-9- (CDK9-), a nucleosome-remodeling-factor-(NURF-), a bromodomain-PHD-finger-transcription-factor- (BPTF-), a ten-eleven-translocation-enzyme-(TET-), a methylcytosine-dioxygenase- (TET1-), histone acetyltransferase (HAT), a histone deacetylase (HDAC), a host-cell-factor-1(HCF1-), an octamer-binding-transcription-factor- (OCT1-), a P-TEFb-, a cyclin-T1-, a PRC2-, a DNA-demethylase, a helicase, an acetyltransferase, a histone-deacetylase, methylated histone lysine protein.

In some embodiments, the second terminus comprises a moiety that binds to an O-linked β-N-acetylglucosamine-transferase (OGT), or CREB binding protein (CBP). In some embodiments, the protein binding moiety is a residue of a molecule that binds to an O-linked β-N-acetylglucosamine-transferase (OGT), or CREB binding protein (CBP).

In some embodiments, the regulatory molecule is a polycomb group (PcG) protein. In certain embodiments, the regulatory molecule is a polycomb repressive complex (PRC). In some embodiments, the regulatory molecule is polycomb repressive complex 1 or polycomb repressive complex 2, PRC1 and PRC2 respectively. In some embodiments, the regulator molecule is a polycomb paralog selected from CBX2, CBX4. CBX6, CBX7, and CBX8.

In some embodiments, the second terminus comprises a moiety that binds to p300/CBP HAT (histone acetyltransferase).

In some embodiments, the second terminus is selected from a bromodomain inhibitor, a BPTF inhibitor, a methylcytosine dioxygenase inhibitor, a DNA demethylase inhibitor, a helicase inhibitor, an acetyltransferase inhibitor, a histone deacetylase inhibitor, a CDK-9 inhibitor, a positive transcription elongation factor inhibitor, and a polycomb repressive complex inhibitor.

In some embodiments, the second terminus is and a CDK9 inhibitor.

In some embodiments, the second terminus is selected from CDK9i, CDK7i, CDK12/13i, Pan-CDKi, a L3MBTL3 recruiter, a CBX recruiter, or an EED recruiter. In some embodiments, the second terminus is CDK9i. In some embodiments, the second terminus is CDK7i. In some embodiments, the second terminus is CDK12/13i. In some embodiments, the second terminus is Pan-CDKi. In some embodiments, the second terminus is a L3MBTL3 recruiter. In some embodiments, the second terminus is a CBX recruiter. In some embodiments, the second terminus is a EED recruiter.

In some embodiments, the second terminus comprises one or more optionally substituted C₆₋₁₀ aryl, optionally substituted C₄₋₁₀ carbocyclic, optionally substituted 4 to 10 membered heterocyclic, or optionally substituted 5 to 10 membered heteroaryl. In some embodiments, the second terminus comprises a diazine or diazepine ring, wherein the diazine or diazepine ring is fused with a C₆₋₁₀ aryl or a 5-10 membered heteroaryl ring comprising one or more heteroatom selected from S, N and O.

In some embodiments, the second terminus comprises an optionally substituted bicyclic or tricyclic structure. In some embodiments, the optionally substituted bicyclic or tricyclic structure comprises a diazepine ring fused with a thiophene ring.

In some embodiments, the second terminus comprises a moiety capable of binding to the regulatory protein, and the moiety is from a compound capable of binding to the regulatory protein.

In some embodiments, the second terminus comprises a compound of Formula (C), or a pharmaceutically acceptable salt or solvate, or hydrate thereof:

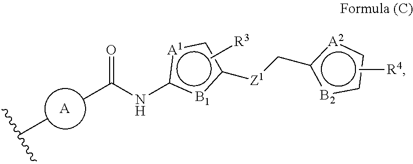

wherein,
Ring A is a 5-10 membered heteroaryl or 5-10 membered heterocycle;
A¹ and A² are each independently CH or N;
B¹ and B² are each independently O, S, or NR⁵;
Z¹ is O, S, or NR⁵;
R³ and R⁴ are each independently hydrogen, halogen, or C₁-C₆ alkyl; and
R⁵ is hydrogen or C₁-C₆ alkyl.

In some embodiments, ring A is a 5, 6, 7, or 8-membered heteroaryl.

In some embodiments, ring A is a 5, 6, 7, or 8-membered heterocycle. In some embodiments, ring A is a 5 membered heterocycle. In some embodiments, ring A is a 6 membered heterocycle. In some embodiments, ring A is a 7 membered heterocycle. In some embodiments, ring A is a piperidine or pyridine.

In some embodiments, A¹ is N and A² is N. In some embodiments, A¹ is N and A² is CH. In some embodiments, A¹ is CH and A² is N.

In some embodiments, B¹ and B² are each independently O or S. In some embodiments, B¹ is S. In some embodiments, B² is O.

In some embodiments, Z¹ is O. In some embodiments, Z¹ is S.

In some embodiments, R³ and R⁴ are each independently C₁-C₆ alkyl. In some embodiments, R³ and R⁴ are each independently hydrogen.

In some embodiments, the second terminus comprises a compound of Formula (C-1), or a pharmaceutically acceptable salt or solvate thereof:

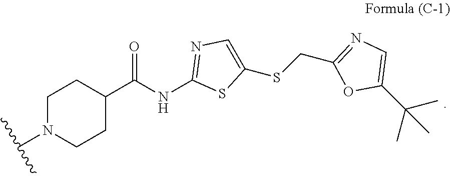 [SMILES: *N1CCC(C(=O)Nc2ncc(SCc3ncc(C(C)(C)C)o3)s2)CC1]

In some embodiments, the second terminus comprises a compound of Formula (D), or a pharmaceutically acceptable salt or solvate thereof:

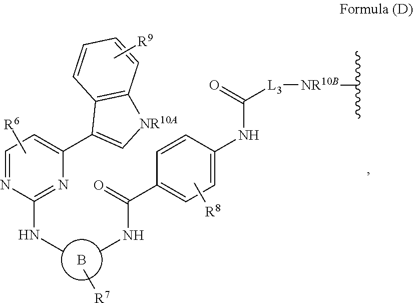

wherein,
ring B is phenyl or 5 to 6-membered cycloalkylene;
L₃ is optionally substituted alkylene or heteroalkylene;
R⁶, R⁷, R⁸, and R⁹ are each independently hydrogen, halogen, optionally substituted C₁₋₆ alkyl, C₁₋₆ haloalkyl, or C₁₋₆ hydroxyalkyl;
R^{10A} is hydrogen, C₁-C₆ alkyl, or SO₂—R^{10C};
R^{10B} is hydrogen or C₁-C₆ alkyl; and
R^{10C} is C₁-C₆ alkyl or phenyl.

In some embodiments, ring B is a phenyl. In some embodiments, ring B is a 5 to 6-membered cycloalkylene (for example a 5 to 6 membered cycloalkyl ring). In some embodiments, ring B is a 5 membered cycloalkylene. In some embodiments, ring B is a 6 membered cycloalkylene.

In some embodiments, L₃ is an optionally substituted alkylene. In some embodiments, L₃ is C3-C6 alkylene. In some embodiments, L₃ is —CHCH—.

In some embodiments, R⁶, R⁷, R⁸, and R⁹ are each independently a halogen. R⁶, R⁷, R⁸, and R⁹ are each independently hydrogen.

In some embodiments, R^{10A} is C₁-C₆ alkyl. In some embodiments, R^{10A} is SO₂—R^{10C}. In some embodiments, R^{10A} is SO₂-phenyl. In some embodiments, R^{10A} is hydrogen.

In some embodiments, R^{10B} is C₁-C₆ alkyl. In some embodiments, R^{10B} is methyl, ethyl or t-butyl. In some embodiments, R^{10B} is hydrogen.

In some embodiments, the second terminus comprises a compound of Formula (D-1), or a pharmaceutically acceptable salt or solvate thereof:

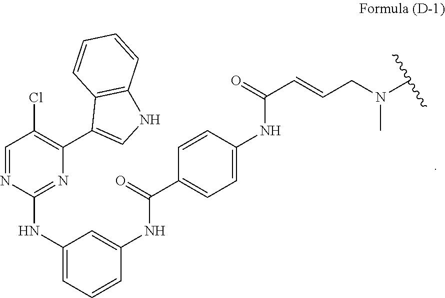 [SMILES: *N(C)C/C=C/C(=O)Nc1ccc(C(=O)Nc2cccc(Nc3ncc(Cl)c(-c4c[nH]c5ccccc45)n3)c2)cc1]

In some embodiments, the second terminus comprises a compound of Formula (D-2), or a pharmaceutically acceptable salt or solvate thereof:

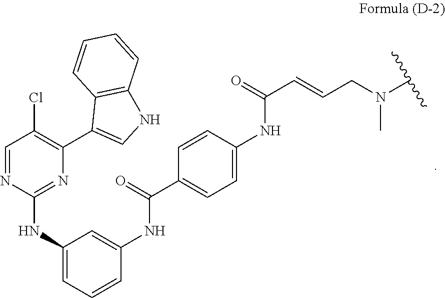

In some embodiments, the second terminus comprises a compound of Formula (D-3), or a pharmaceutically acceptable salt or solvate thereof:

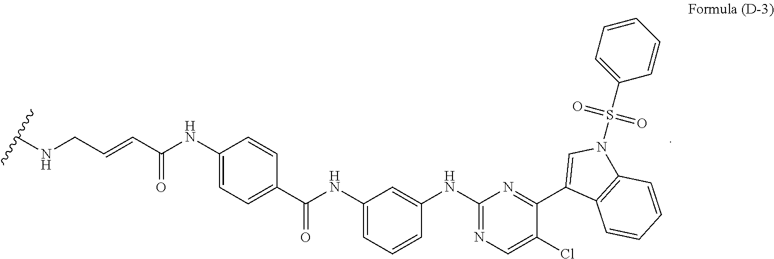 [SMILES: *NC/C=C/C(=O)Nc1ccc(C(=O)Nc2cccc(Nc3ncc(Cl)c(-c4cn(S(=O)(=O)c5ccccc5)c5ccccc45)n3)c2)cc1]

In some embodiments, the second terminus comprises a compound of Formula (D-4), or a pharmaceutically acceptable salt or solvate thereof:

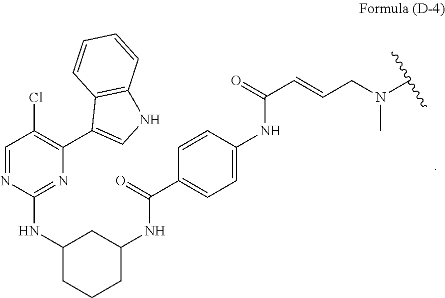 [SMILES: *N(C)C/C=C/C(=O)Nc1ccc(C(=O)NC2CCCC(Nc3ncc(Cl)c(-c4c[nH]c5ccccc45)n3)C2)cc1]

In some embodiments, the second terminus comprises a compound of Formula (E), or a pharmaceutically acceptable salt or solvate thereof:

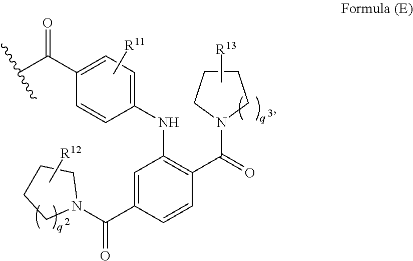

wherein,
q² and q³ are each independently 1, 2, 3, or 4;
R¹¹ is hydrogen, halogen, optionally substituted C₁₋₆ alkyl, C₁₋₆ haloalkyl, or C₁₋₆ hydroxyalkyl; and
each R¹² and R¹³ is independently an optionally substituted 5-8 membered heterocycloalkyl.

In some embodiments, R¹¹ is an optionally substituted C₁₋₆ alkyl, C₁₋₆ haloalkyl, or C₁₋₆ hydroxyalkyl. In some embodiments, R¹¹ is halogen. In some embodiments, R¹¹ is hydrogen.

In some embodiments, each R¹² and R¹³ is independently an optionally substituted 5-membered heterocycloalkyl ring. In some embodiments, each R¹² and R¹³ is independently an optionally substituted 6-membered heterocycloalkyl ring. In some embodiments, each R¹² and R¹³ is independently an optionally substituted 6-membered heterocycloalkyl ring. In some embodiments, each R¹² and R¹³ is independently an optionally substituted 7-membered heterocycloalkyl ring.

In some embodiments, q2 and q3 are each independently 1, 2, or 3. In some embodiments, q2 and q3 are each independently 1. In some embodiments, q2 and q3 are each independently 2.

In some embodiments, the second terminus comprises a compound of Formula (E-1), or a pharmaceutically acceptable salt or solvate thereof:

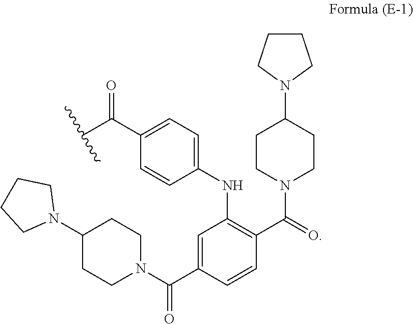 [SMILES: *C(=O)c1ccc(Nc2cc(C(=O)N3CCC(N4CCCC4)CC3)ccc2C(=O)N2CCC(N3CCCC3)CC2)cc1]

In some embodiments, the second terminus comprises a compound of Formula (F), or a pharmaceutically acceptable salt or solvate thereof:

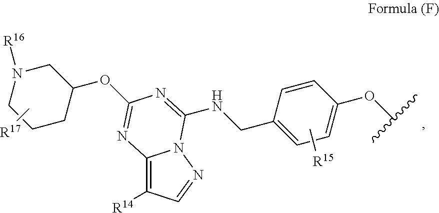

wherein,
R¹⁴ and R¹⁷ are each independently hydrogen, halogen, optionally substituted C₁₋₂₀ alkyl, C₁₋₂₀ heteroalkyl, C₁₋₆ haloalkyl, or C₁₋₆ hydroxyalkyl;
R¹¹ is an optionally substituted 5 membered heteroaryl; and
R¹⁶ is hydrogen or C₁-C₆ alkyl.

In some embodiments, R¹⁴ is an optionally substituted C₁₋₂₀ alkyl, C₁₋₆ haloalkyl, or C₁₋₆ hydroxyalkyl. In some embodiments, R¹⁴ is C₁₋₂₀ heteroalkyl. In some embodiments, the heteroalkyl is PEG. In some embodiments, the PEG comprises 1-20 PEG units. In some embodiments, R¹⁴ halogen. In some embodiments, R¹⁷ hydrogen.

In some embodiments, R¹¹ is an optionally 5-membered heteroaryl comprising 1, 2, or 3 nitrogen atoms.

In some embodiments, R¹⁷ is an optionally substituted C₁₋₆ alkyl, C₁₋₆ haloalkyl, or C₁₋₆ hydroxyalkyl. In some embodiments, R¹⁷ halogen. In some embodiments, R¹⁷ hydrogen.

In some embodiments, the second terminus comprises a compound of Formula (F-1), or a pharmaceutically acceptable salt or solvate thereof:

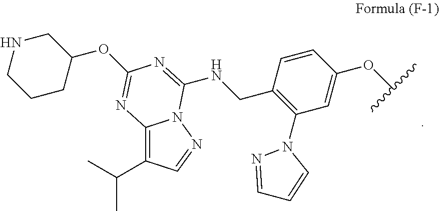 [SMILES: *Oc1ccc(CNc2nc(OC3CCCNC3)nc3c(C(C)C)cnn23)c(-n2cccn2)c1]

In some embodiments, the second terminus comprises a compound of Formula (G), or a pharmaceutically acceptable salt or solvate thereof:

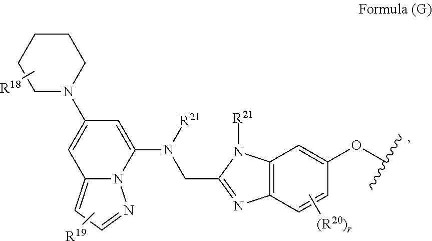

wherein,
r is 0, 1, or 2;
R¹⁸ and R¹⁹ are each independently hydrogen, optionally substituted C₁₋₂₀ alkyl, C₁₋₂₀ heteroalkyl, C₁₋₆ haloalkyl, or C₁₋₆ hydroxyalkyl;
each R²⁰ is independently hydrogen, halogen, or C₁-C₆ alkyl; and
each R²¹ is independently hydrogen or C₁-C₆ alkyl.

In some embodiments, R¹⁸ is an optionally substituted C₁₋₂₀ alkyl, C₁₋₆ haloalkyl, or C₁₋₆ hydroxyalkyl. In some embodiments, R¹⁸ is an optionally substituted C₁₋₆ alkyl. In some embodiments, R¹⁸ is an optionally substituted C₁₋₂₀ heteroalkyl. In some embodiments, the heteroalkyl is PEG. In some embodiments, the PEG comprises 1-20 PEG units.

In some embodiments, R¹⁹ is an optionally substituted C₁₋₆ alkyl, C₁₋₆ haloalkyl, or C₁₋₆ hydroxyalkyl. In some embodiments, R¹⁹ halogen. In some embodiments, R¹⁹ hydrogen. In some embodiments, R¹⁸ is an optionally substituted C₁₋₂₀ heteroalkyl. In some embodiments, the heteroalkyl is PEG. In some embodiments, the PEG comprises 1-20 PEG units.

In some embodiments, each R²⁰ is independently C₁-C₆ alkyl. In some embodiments, each R²⁰ is independently halogen.

In some embodiments, r is 1 or 2. In some embodiments, r is 0.

In some embodiments, the second terminus comprises a compound of Formula (G-1), or a pharmaceutically acceptable salt or solvate thereof:

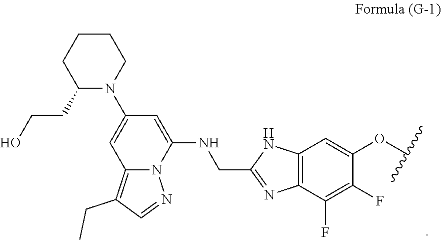 [SMILES: *Oc1cc2[nH]c(CNc3cc(N4CCCC[C@H]4CCO)cc4c(CC)cnn34)nc2c(F)c1F]

In some embodiments, the second terminus comprises a compound of Formula (H-1), or a pharmaceutically acceptable salt or solvate thereof:

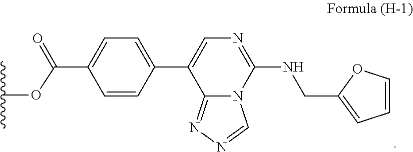 [SMILES: *OC(=O)c1ccc(-c2cnc(NCc3ccco3)n3cnnc23)cc1]

In some embodiments, the second terminus comprises a compound of Formula (H-2), or a pharmaceutically acceptable salt or solvate thereof:

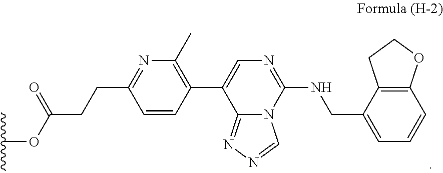 [SMILES: *OC(=O)CCc1ccc(-c2cnc(NCc3cccc4c3CCO4)n3cnnc23)c(C)n1]

In some embodiments, the second terminus comprises a compound of Formula (J), or a pharmaceutically acceptable salt or solvate thereof:

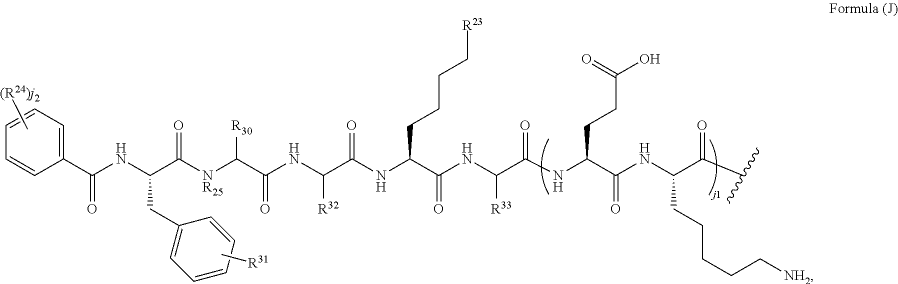

wherein,
R²³ is —NR^{23A}R^{23B} or —NR^{23A}(R^{23B})₂; wherein
   R^{23A} and R^{23B} are each independently an optionally substituted C₁₋₆ alkyl, C₃-C₁₀ cycloalkyl, aryl or heteroaryl; or
   R^{23A} and R^{23B} are joined together with the nitrogen to which they are attached to form a heterocyclic ring;
R²⁴ is hydrogen, halogen, C₁₋₆ alkyl, C₁₋₆ haloalkyl, or C₁₋₆ alkoxy;
R²¹ is hydrogen or C₁₋₃ alkyl;
R³⁰, R³², and R³³ are each independently hydrogen, halogen, optionally substituted C₁₋₆ alkyl, C₁₋₆ alkoxy, or C₃-C₆ cycloalkyl ring;
R³¹ is C₁₋₆ alkyl or C₃₋₁₀ cycloalkyl;
j₁ is 0 or 1; and
j₂ is 0, 1, 2, or 3.

In some embodiments, j₁ is 0. In some embodiments, j₁ is 1.

In some embodiments, j₂ is 0, in some embodiments, j₂ is 1. In some embodiments, j₂ is 2.

In some embodiments, the second terminus comprises a compound of Formula (J-1), or a pharmaceutically acceptable salt or solvate thereof:

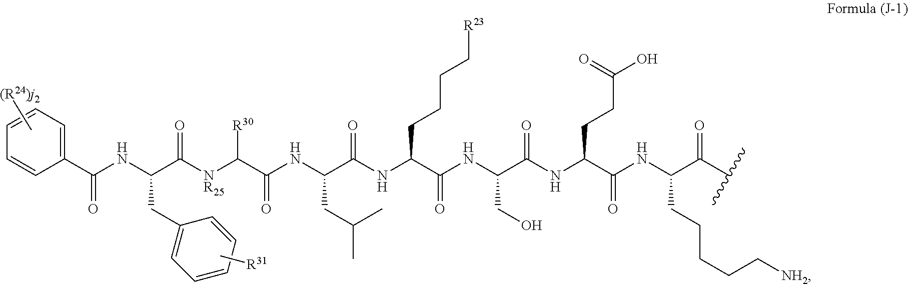

wherein,
R²³ is —NR^{23A}R^{23B} or —NR^{23A}(R^{23B})₂; wherein
   R^{23A} and R^{23B} are each independently an optionally substituted C₁₋₆ alkyl, C₃-C₁₀ cycloalkyl, aryl or heteroaryl; or
   R^{23A} and R^{23B} are joined together with the nitrogen to which they are attached to form a heterocyclic ring;
R²⁴ is hydrogen, halogen, C₁₋₆ alkyl, C₁₋₆ haloalkyl, or C₁₋₆ alkoxy;
R²¹ is hydrogen or C₁₋₃ alkyl;
R³⁰ is hydrogen, halogen, C₁₋₆ alkyl, or C₁₋₆ alkoxy; and
R³¹ is C₁₋₆ alkyl or C₃₋₁₀ cycloalkyl.

In some embodiments, the second terminus comprises a compound of Formula (J-2), or a pharmaceutically acceptable salt or solvate thereof:

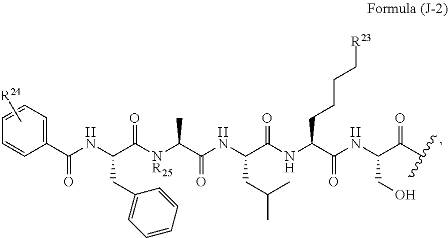

wherein,
R²³ is —NR^{23A}R^{23B} or —NR^{23A}(R^{23B})₂; wherein
   R^{23A} and R^{23B} are each independently an optionally substituted C₁₋₆ alkyl, C₃-C₁₀ cycloalkyl, aryl or heteroaryl; or
   R^{23A} and R^{23B} are joined together with the nitrogen to which they are attached to form a heterocyclic ring;
R²⁴ is hydrogen, halogen, C₁₋₆ alkyl, C₁₋₆ haloalkyl, or C₁₋₆ alkoxy; and
R²¹ is hydrogen or C₁₋₃ alkyl.

In some embodiments, R^{23A} and R^{23B} are independently an optionally substituted C₁₋₆ alkyl. In some embodiments, the alkyl is —CH₃, —CH₂CH₃, —CH₂CH₂CH₃, or —CH(CH₃)₂. In some embodiments, R^{23A} and R^{23B} are independently an optionally substituted C₃-C₁₀ cycloalkyl. In some embodiments, the cycloalkyl is a monocyclic or bicyclic cycloalkyl. In some embodiments, the cycloalkyl is cyclobutyl, cyclopentyl, cyclohexyl, or adamantly. In some embodiments, R^{23A} and R^{23B} are independently an optionally substituted aryl. In some embodiments, the aryl is a phenyl.

In some embodiments, —NR^{23A}R^{23B} is

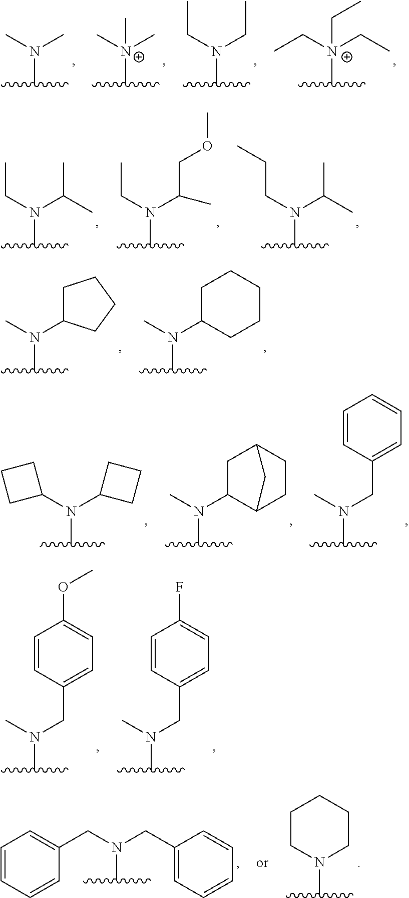

In some embodiments, R²⁴ is C₁₋₆ alkyl. In some embodiments, R²⁴ is —CH₃, —CH₂CH₃, —CH(CH₃)₂, or —C(CH₃)₃. In some embodiments, R²⁴ is halogen. In some embodiments, R²⁴ is —Br, —Cl, —F, or —I. In some embodiments, R²⁴ is —CF₃ or —OCH₃. In some embodiments, R²⁴ is hydrogen.

In some embodiments, the second terminus comprises a compound of Formula (J-3), or a pharmaceutically acceptable salt or solvate thereof:

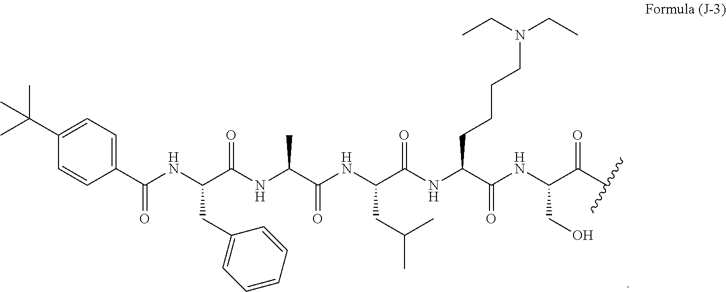 [SMILES: *C(=O)[C@H](CO)NC(=O)[C@H](CCCCN(CC)CC)NC(=O)[C@H](CC(C)C)NC(=O)[C@H](C)NC(=O)[C@H](Cc1ccccc1)NC(=O)c1ccc(C(C)(C)C)cc1]

In some embodiments, the second terminus comprises a compound of Formula (J-4), or a pharmaceutically acceptable salt or solvate thereof:

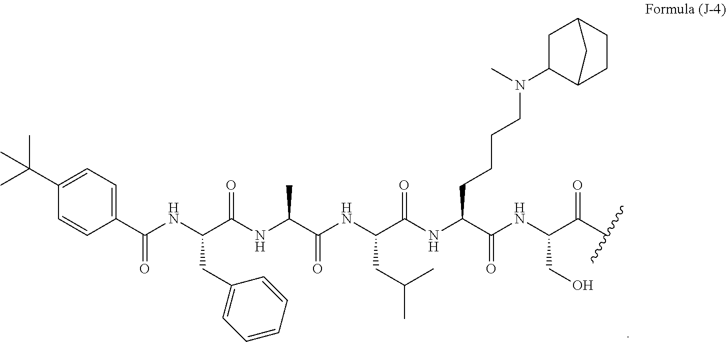 [SMILES: *C(=O)[C@H](CO)NC(=O)[C@H](CCCCN(C)C1CC2CCC1C2)NC(=O)[C@H](CC(C)C)NC(=O)[C@H](C)NC(=O)[C@H](Cc1ccccc1)NC(=O)c1ccc(C(C)(C)C)cc1]

In some embodiments, R³⁰, R³², and R³³ are each independently hydrogen. In some embodiments, R³⁰, R³², and R³³ are each independently halogen. In some embodiments, R³⁰, R³², and R³³ are each independently hydrogen an optionally substituted C₁₋₆ alkyl, C₁₋₆ alkoxy, or C₃-C₆ cycloalkyl ring. In some embodiments, R³⁰, R³², and R³³ are each independently methyl, ethyl, n-propyl, isopropyl, n-butyl, sec-butyl, iso-butyl, or tert-butyl. In some embodiments, R³⁰, R³², and R³³ are each independently a cyclobutyl, cyclopentyl, or cyclohexyl ring.

In some embodiments, the second terminus comprises a compound of Formula (J-5), or a pharmaceutically acceptable salt or solvate thereof:

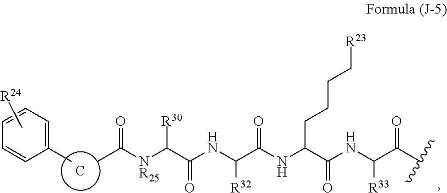

wherein,
Ring C is an optionally substituted 5 to 6 membered heterocyclyl ring;
R²³ is —NR^{23A}R^{23B} or —NR^{23A}(R^{23B})₂; wherein
   R^{23A} and R^{23B} are each independently an optionally substituted C₁₋₆ alkyl, C₃-C₁₀ cycloalkyl, aryl or heteroaryl; or
   R^{23A} and R^{23B} are joined together with the nitrogen to which they are attached to form a heterocyclic ring;
R²⁴ is hydrogen, halogen, C₁₋₆ alkyl, C₁₋₆ haloalkyl, or C₁₋₆ alkoxy;
R²¹ is hydrogen or C₁₋₃ alkyl; and
R³⁰, R³², and R³³ are each independently hydrogen, halogen, optionally substituted C₁₋₆ alkyl, C₁₋₆ alkoxy, or C₃-C₆ cycloalkyl ring.

In some embodiments, ring C is a 5-membered heterocyclyl ring. In some embodiments, ring C is a 5-membered heterocyclyl ring comprising 1 to 3 heteroatoms selected from N, S, and O.

In some embodiments, the second terminus comprises a compound of Formula (J-6), or a pharmaceutically acceptable salt or solvate thereof:

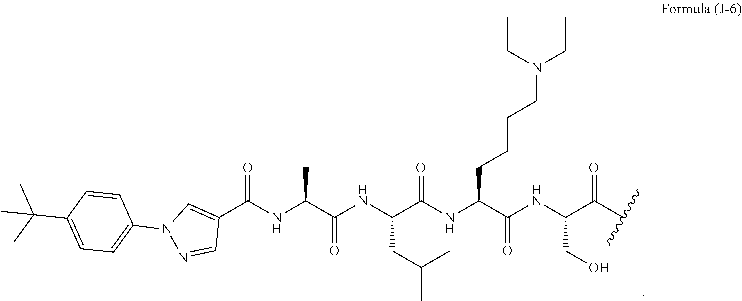

In some embodiments, the second terminus comprises a compound of Formula (J-7), or a pharmaceutically acceptable salt or solvate thereof:

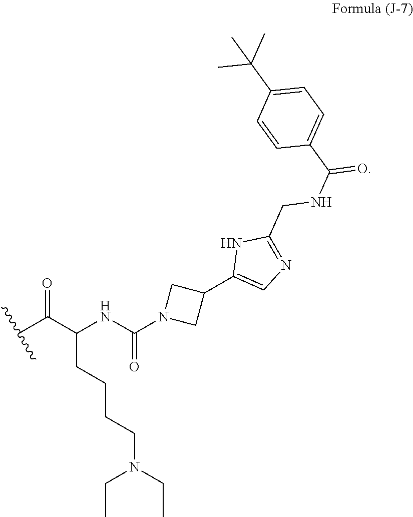 [SMILES: *C(=O)C(CCCCN(CC)CC)NC(=O)N1CC(c2cnc(CNC(=O)c3ccc(C(C)(C)C)cc3)[nH]2)C1]

In some embodiments, the second terminus comprises a compound of Formula (J-8), or a pharmaceutically acceptable salt or solvate thereof:

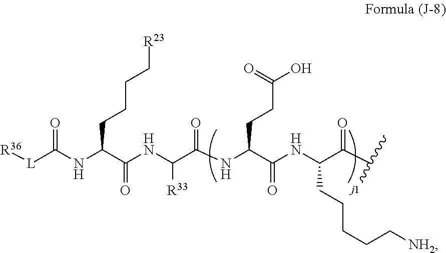

wherein,
L is absent or an optionally substituted C₁₋₂₀ alkylene or C₁₋₂₀ heteroalkylene linker;
R²³ is —NR^{23A}R^{23B} or —NR^{23A}(R^{23B})₂; wherein
   R^{23A} and R^{23B} are each independently an optionally substituted C₁₋₆ alkyl, C₃-C₁₀ cycloalkyl, aryl or heteroaryl; or
   R^{23A} and R^{23B} are joined together with the nitrogen to which they are attached to form a heterocyclic ring;
R³³ are each independently hydrogen, halogen, optionally substituted C₁₋₆ alkyl, C₁₋₆ alkoxy, or C₃-C₆ cycloalkyl ring; and
R³⁶ is hydrogen, —C(O)R³⁷ or —NR³⁸C(O)R³⁷;
R³⁷ is an optionally substituted aryl, C₁₋₆ alkyl, or C₃-C₆ cycloalkyl ring;
R³⁸ is hydrogen or C₁₋₆ alkyl; and
j₁ is 0 or 1.

In some embodiments, L is absent. In some embodiments, L is an optionally substituted C₁₋₂₀ alkylene linker. In some embodiments, L is an optionally substituted C₁₋₂₀ heteroalkylene linker. In some embodiments, the heteroalkylene linker is a PEG linker. In some embodiments, the PEG has 1-20 PEG units.

In some embodiments, R³⁶ is hydrogen. In some embodiments, R³⁶ is —C(O)R³⁷ or —NR³⁸C(O)R³⁷. In some embodiments, R³⁷ is an optionally substituted aryl. In some embodiments, R³⁷ is an optionally substituted phenyl, optionally substituted with 1, 2 or 3 halogen, C₁₋₆ alkyl, C₁₋₆ haloalkyl, or C₁₋₆ alkoxy. In some embodiments, R³⁷ is phenyl optionally substituted with C₁₋₆ alkyl.

In some embodiments, the second terminus comprises a compound of Formula (K), or a pharmaceutically acceptable salt or solvate thereof:

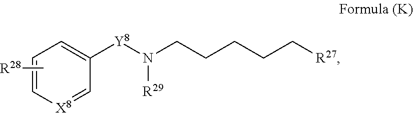

wherein,
X⁸ is CH or N;
Y⁸ is —C(O)—, or —S(O)₂—;
R²⁷ is an optionally substituted cation of C₁₋₆ alkyl, C₃-C₁₀ cycloalkyl, or 5 to 10-membered heteroaryl;
R²⁸ is hydrogen, halogen, or C₁₋₆ alkyl; and
R²⁹ is hydrogen or C₁₋₃ alkyl.

In some embodiments, Y⁸ is —C(O)—. In some embodiments, Y⁸ is —S(O)₂—.

In some embodiments, the second terminus comprises a compound of Formula (K-1) or (K-2), or a pharmaceutically acceptable salt or solvate thereof:

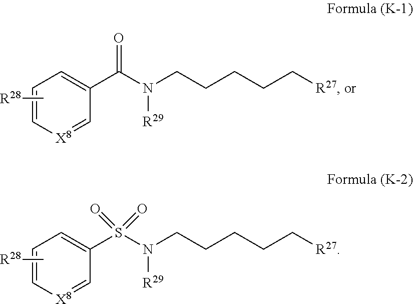

In some embodiments, X⁸ is CH. In some embodiments, X⁸ is N.

In some embodiments, R²⁷ is selected from

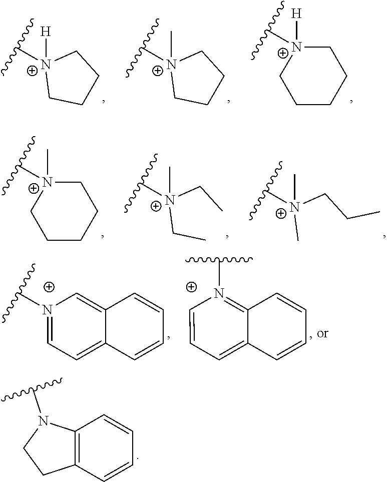

In some embodiments, R²⁸ is halogen. In some embodiments, R²⁸ is —Br, —Cl, —F, or —I. In some embodiments, R²⁸ is hydrogen.

In some embodiments, the second terminus comprises a compound selected from:

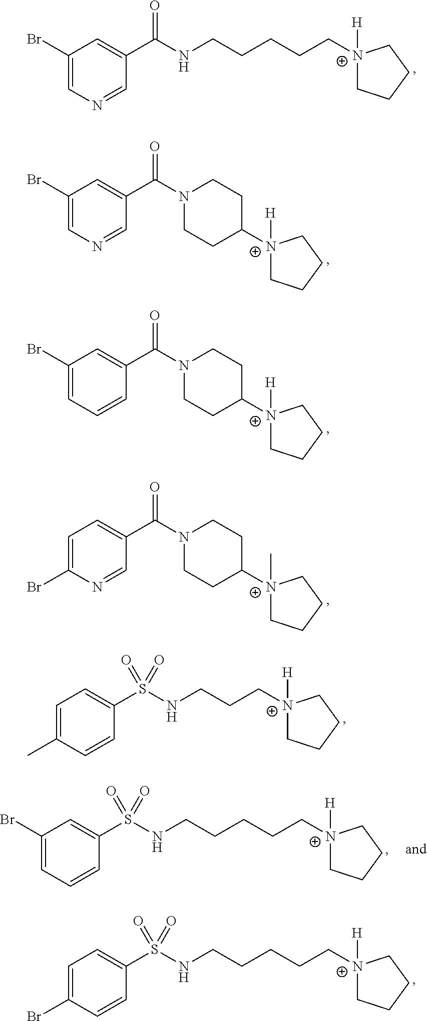

or a pharmaceutically acceptable salt or solvate thereof.

In some embodiments, the second terminus comprises a compound of Formula (L), or a pharmaceutically acceptable salt or solvate thereof:

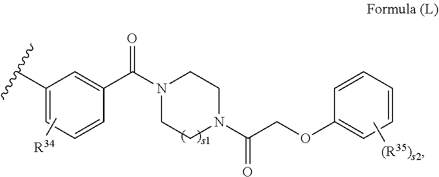

wherein,
R³³ is halogen, C₁₋₆ alkyl, C₁₋₆ haloalkyl, or C₁₋₆ hydroxyalkyl;
R³⁵ is halogen, optionally substituted C₁₋₆ alkyl, C C₁₋₆ aminoalkyl₃-C₁₀ cycloalkyl, or 5 to 10-membered heteroaryl; and
s1 and s2 are each independently 1, 2, 3, or 4.

In some embodiments, the second terminus comprises a compound selected from:

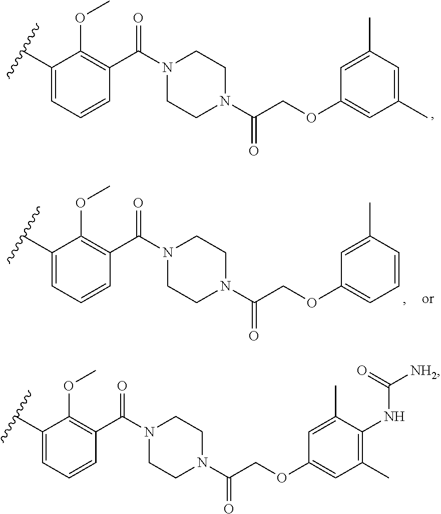 [SMILES: *c1cccc(C(=O)N2CCN(C(=O)COc3cc(C)cc(C)c3)CC2)c1OC ; *c1cccc(C(=O)N2CCN(C(=O)COc3cccc(C)c3)CC2)c1OC ; *c1cccc(C(=O)N2CCN(C(=O)COc3cc(C)c(NC(N)=O)c(C)c3)CC2)c1OC]

or O or a pharmaceutically acceptable salt or solvate thereof.

In some embodiments, the second terminus comprises

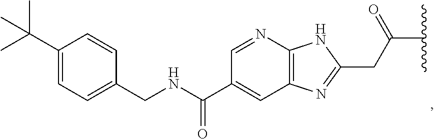 [SMILES: *C(=O)Cc1nc2cc(C(=O)NCc3ccc(C(C)(C)C)cc3)cnc2[nH]1]

or a pharmaceutically acceptable salt or solvate thereof.

In some embodiments, the second terminus comprises

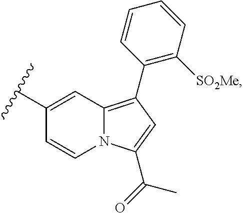 [SMILES: *c1ccn2c(C(C)=O)cc(-c3ccccc3S(C)(=O)=O)c2c1]

or a pharmaceutically acceptable salt or solvate thereof.

In some embodiments, the second terminus comprises

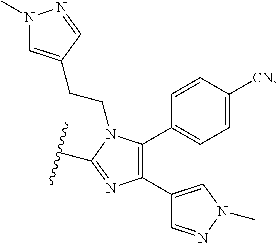 [SMILES: *c1nc(-c2cnn(C)c2)c(-c2ccc(C#N)cc2)n1CCc1cnn(C)c1]

or a pharmaceutically acceptable salt or solvate thereof.

In some embodiments, the second terminus does not comprises JQ1, iBET762, OTX015, RVX208, or AU1. In some embodiments, the second terminus does not comprises JQ1. In some embodiments, the second terminus does not comprises a moiety that binds to a bromodomain protein. In some embodiments, the second terminus does not comprises JQ1, JQ-1, OTX015, RVX208 acid, or RVX208 hydroxyl.

In certain embodiments, the regulatory molecule is not a bromodomain-containing protein chosen from BRD2, BRD3, BRD4, and BRDT. In certain embodiments, the regulatory molecule is not BRD2, BRD3, BRD4, or BRDT

In some embodiments, the protein binding moiety is not

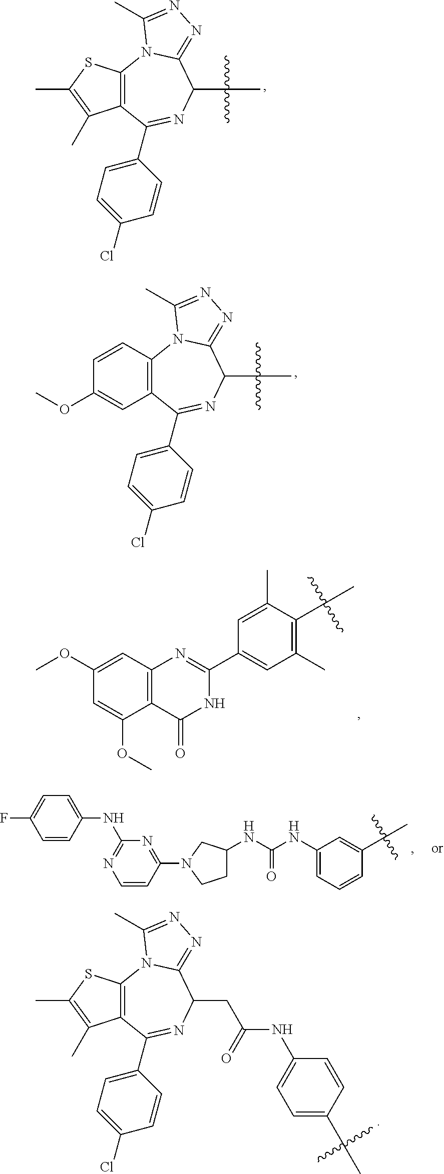

The protein binding moiety can include a residue of a compound that binds to a regulatory protein.

In certain embodiments, the regulatory molecule is a transcription factor.

In certain embodiments, the regulatory molecule is an RNA polymerase.

In certain embodiments, the regulatory molecule is a moiety that regulates the activity of RNA polymerase.

In certain embodiments, the regulatory molecule interacts with TATA binding protein.

In certain embodiments, the regulatory molecule interacts with transcription factor II D.

In certain embodiments, the regulatory molecule comprises a CDK9 subunit.

In certain embodiments, the regulatory molecule is P-TEFb.

In certain embodiments, the recruiting moiety binds to the regulatory molecule but does not inhibit the activity of the regulatory molecule. In certain embodiments, the recruiting moiety binds to the regulatory molecule and inhibits the activity of the regulatory molecule. In certain embodiments, the recruiting moiety binds to the regulatory molecule and increases the activity of the regulatory molecule.

In certain embodiments, the recruiting moiety binds to the active site of the regulatory molecule. In certain embodiments, the recruiting moiety binds to a regulatory site of the regulatory molecule.

### Oligomeric Backbone Linker

The Oligomeric backbone contains a linker that connects the first terminus and the second terminus and brings the regulatory molecule in proximity to the target gene to modulate gene expression.

The length of the linker depends on the type of regulatory protein and also the target gene. In some embodiments, the linker has a length of less than about 50 Angstroms. In some embodiments, the linker has a length of about 20 to 30 Angstroms.

In some embodiments, the linker comprises between 5 and 50 chain atoms.

In some embodiments, the linker comprises a multimer having 2 to 50 spacing moieties, wherein the spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₋₁₂ alkyl, optionally substituted C₂₋₁₀ alkenyl, optionally substituted C₂₋₁₀ alkynyl, optionally substituted C₆₋₁₀ arylene, optionally substituted C₃₋₇ cycloalkylene, optionally substituted 5- to 10-membered heteroarylene, optionally substituted 4- to 10-membered heterocycloalkylene, amino acid residue, —O—, —C(O)NR^{4a}—, —NR^{4a}C(O)—, —C(O)—, —NR¹—, —C(O)O—, —O—, —S—, —S(O)—, —SO₂—, —SO₂NR^{4a}—, —NR^{4a}SO₂—, and —P(O)OH—, and any combinations thereof; wherein
each x is independently 2-4;
each y is independently 1-10;
each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁₋₆ alkyl.

In some embodiments, the oligomeric backbone comprises -(T¹-V¹)ₐ-(T²-V²)_{b}-(T³-V³)_{c}-(T⁴-V⁴)_{d}-(T⁵-V⁵)ₑ-,
wherein a, b, c, d and e are each independently 0 or 1, and where the sum of a, b, c, d and e is 1 to 5;
T¹, T², T³, T⁴ and T⁵ are each independently selected from an optionally substituted (C₁-C₁₂)alkylene, optionally substituted alkenylene, optionally substituted alkynylene, (EA)_{w}, (EDA)ₘ, (PEG)ₘ, (modified PEG)ₙ, (AA)ₚ, —(CR^{2a}OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, an acetal group, a disulfide, a hydrazine, a carbohydrate, a beta-lactam, and an ester,
   (a) w is an integer from 1 to 20;
   (b) m is an integer from 1 to 20;
   (c) n is an integer from 1 to 30;
   (d) p is an integer from 1 to 20;
   (e) h is an integer from 1 to 12;
(f) EA has the following structure

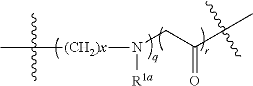

(g) EDA has the following structure:

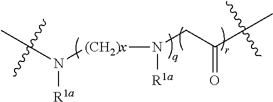

wherein each q is independently an integer from 1 to 6, each x is independently an integer from 1 to 4, and each r is independently 0 or 1;
(h) (PEG)ₙ has the structure of —(CR^{2a}R^{2b}—CR^{2a}R^{2b}—O)ₙ—CR^{2a}R^{2b}—; —
(i) (modified PEG), has the structure of replacing at least one —(CR^{2a}R^{2b}—CR^{2a}R^{2b}—O)— in (PEG), with —(CH₂—CR^{2a}═CR^{2a}—CH₂—O)— or —(CR^{2a}R^{2b}—CR^{2a}R^{2b}—S)—;
(j) AA is an amino acid residue;
(k) V¹, V², V³, V⁴ and V⁵ are each independently selected from the group consisting of a bond, CO—, —NR^{1a}—, —CONR^{1a}—, —NR^{1a}CO—, —CONR^{1a}C₁₋₄ alkyl-, —NR^{1a}CO—C₁₋₄ alkyl-, —C(O)O—, —OC(O)—, —O—, —S—, —S(O)—, —SO₂—, —SO₂NR^{1a}—, —NR^{1a}SO₂— and —P(O)OH—;
(l) each R^{1a} is independently hydrogen or and optionally substituted C₁₋₆ alkyl; and each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, the a, b, c, d and e are each independently 0 or 1, where the sum of a, b, c, d and e is 1. In some embodiments, the a, b, c, d and e are each independently 0 or 1, where the sum of a, b, c, d and e is 2. In some embodiments, the a, b, c, d and e are each independently 0 or 1, where the sum of a, b, c, d and e is 3. In some embodiments, the a, b, c, d and e are each independently 0 or 1, where the sum of a, b, c, d and e is 4. In some embodiments, the a, b, c, d and e are each independently 0 or 1, where the sum of a, b, c, d and e is 5.

In some embodiments, n is 3-9. In some embodiments, n is 4-8. In some embodiments, n is 5 or 6.

In some embodiments, T, T², T³, and T⁴, and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, phenyl, substituted phenyl, piperidin-4-amino (P4A), para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ, In some embodiments, piperidin-4-amino (P4A) is

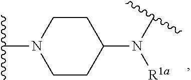

wherein R^{1a} is H or C₁₋₆alkyl.

In some embodiments, T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, optionally substituted 5-10 membered heteroarylene. In some embodiments, EA has the following structure:

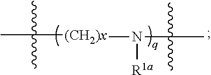

and
EDA has the following structure:

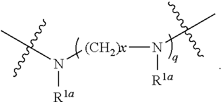

In some embodiments, x is 2-3 and q is 1-3 for EA and EDA. In some embodiments, R^{1a} is H or C₁₋₆ alkyl.

In some embodiments, T⁴ or T⁵ is an optionally substituted (C₆-C₁₀) arylene.

In some embodiments, T⁴ or T⁵ is phenylene or substituted phenylene. In some embodiments, T⁴ or T⁵ is phenylene or phenylene substituted with 1-3 substituents selected from —C₁₋₆ alkyl, halogen, OH or amine. In some embodiments, T⁴ or T⁵ is 5-10 membered heteroarylene or substituted heteroarylene. In some embodiments, T⁴ or T⁵ is 4-10 membered heterocyclene or substituted heterocyclylene. In some embodiments, T⁴ or T⁵ is heteroarylene or heterocyclene optionally substituted with 1-3 substituents selected from —C₁₋₆ alkyl, halogen, OH or amine.

In some embodiments, T¹, T², T³, T⁴ and T⁵ and V¹, V², V³, V⁴ and V⁵ are selected from the following Table 2.

TABLE 2

| T¹ | V¹ | T² | V² | T³ | V³ | T⁴ | V⁴ | T⁵ | V⁵ |
|---|---|---|---|---|---|---|---|---|---|
| (C₁-C₁₂) | CONR^{1a} | (EA)_{w} | CO | (PEG)ₙ | NR¹¹CO | — | — | — | — |
| alkylene | | | | | | | | | |
| (C₁-C₁₂) | CONR^{1a} | (EA)_{w} | CO | (PEG)ₙ | O | arylene | NR¹¹CO | — | — |
| alkylene | | | | | | | | | |
| (C₁-C₁₂) | CONR^{1a} | (EA)_{w} | CO | (PEG)ₙ | O | Subst. | NR¹¹CO | — | — |
| alkylene | | | | | | arylene | | | |
| (C₁-C₁₂) | CONR^{1a} | (EA)_{w} | CO | (PEG)ₙ | O | NR¹¹CO | (C₁-C₁₂) | Subst. | NR¹¹CO |
| alkylene | | | | | | | alkyl | arylene | |
| (C₁-C₁₂) | CONR^{1a} | (EA)_{w} | CO | (C₁-C₁₂) | NR¹¹CO- | Subst. | NR¹¹ | — | — |
| alkylene | | | | alkyl | C₁₋₄ alkyl | arylene | | | |
| (C₁-C₁₂) | CONR^{1a} | (EA)_{w} | CO | (PEG)ₙ | O | Subst. | — | — | — |
| alkylene | | | | | | arylene | | | |
| (PEG)ₙ | CONR^{1a-} | — | — | — | — | — | — | — | — |
| | C₁₋₄ alkyl | | | | | | | | |
| (EA)_{w} | CO | (C₁-C₁₂) | CONR¹¹⁻ | — | — | — | — | — | — |
| | | alkyl | C₁₋₄ alkyl | | | | | | |
| (C₁-C₁₂) | CONR^{1a} | (EA)_{w} | CO | (PEG)ₙ | NR¹¹CO- | — | — | — | — |
| alkylene | | | | | C₁₋₄ alkyl | | | | |
| (EA)_{w} | CO | (PEG)ₙ | O | phenyl | NR¹¹CO- | — | — | — | — |
| | | | | | C₁₋₄ alkyl | | | | |
| (C₁-C₁₂) | CONR^{1a} | (PEG)ₙ | CO | — | — | — | — | — | — |
| alkylene | | | | | | | | | |
| (C₁-C₁₂) | CONR^{1a} | (EA)_{w} | CO | modifd. | O | arylene | NR¹¹CO | — | — |
| alkylene | | | | (PEG)ₙ | | | | | |

In some embodiments, the linker comprises N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

In some embodiments, the linker comprises the linker comprises —(CH₂—C(O)N(R″)—(CH₂)_{q}—N(R′)—(CH₂)_{q}—N(R″)C(O)—(CH₂)ₓ—C(O)N(R″)-A-, —(CH₂)ₓ—C(O)N(R″)—(CH₂CH₂O)_{y}(CH₂)ₓ—C(O)N(R″)-A-, —C(O)N(R″)—(CH₂)_{q}—N(R′)—(CH₂)_{q}—N(R″)C(O)—(CH₂)ₓ-A-, —(CH₂)ₓ—O—(CH₂CH₂O)_{y}—(CH₂)ₓ—N(R″)C(O)—(CH₂)ₓ-A-, or —N(R″)C(O)—(CH₂)—C(O)N(R″)—(CH₂)ₓ—O(CH₂CH₂O)_{y}(CH₂)ₓ-A-; wherein R′ is methyl; R″ is hydrogen; each x and y are independently an integer from 1 to 10; each q is independently an integer from 2 to 10; and each A is independently selected from a bond, an optionally substituted C₁₋₁₂ alkyl, an optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

In some embodiments, the linker comprises —(CH₂CH₂—O)ₓ₁— or —(CH₂CH₂—O)ₓ₂-A-(CH₂CH₂—O)ₓ₃—, wherein A is an optionally substituted 4- to 10-membered heterocycloalkylene or spirocyclene, and each x1, x2, and x3 is independently an integer from 1-15.

In some embodiments, the linker comprises polyethylene glycol (PEG). In some embodiments, the linker comprises 1-20 PEG unites. In some embodiments, the linker comprises 1, 2, 3, 4, 5, 6, 7, 8, 9, 10, 11, 12, 13, 14, 15, 16, 17, 18, 19, or 20 PEG units.

In some embodiments, A is selected from

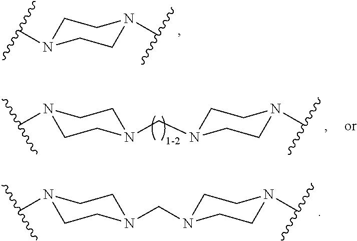

In some embodiments, A is

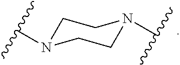 [SMILES: *N1CCN(*)CC1]

In some embodiments, A is

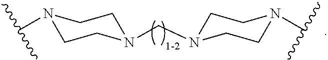

In some embodiments, A is

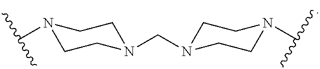 [SMILES: *N1CCN(CN2CCN(*)CC2)CC1]

In some embodiments, the linker is joined with the first terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR¹SO₂—, —P(O)OH—, —((CH₂)ₓ—O)—, —((CH₂)_{y}—NR^{a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁₋₆ alkyl.

In some embodiments, the linker is joined with the first terminus with a group selected from —CO—, —NR^{1a}—, C₁₋₁₂ alkyl, —CONR^{1a}—, and —NR^{1a}CO—.

In some embodiments, the linker is joined with second terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR¹SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁₋₆ alkyl.

In some embodiments, the linker is joined with second terminus with a group selected from —CO—, —NR^{1a}—, —CONR^{1a}—, —NR^{1a}CO—, —((CH₂)ₓ—O)—, —((CH₂)_{y}—NR^{1a})—, —O—, optionally substituted —C₁₋₁₂ alkyl, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R¹ is independently a hydrogen or optionally substituted C₁₋₆ alkyl.

In some embodiments, the linker comprises a structure of Formula (C-1), or a pharmaceutically acceptable salt or solvate thereof:

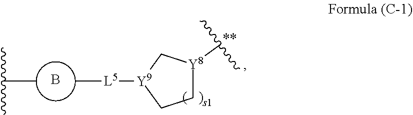

wherein,
Ring B is absent, arylene or heterocycloalkylene;
L⁵ is absent, optionally substituted alkylene or alkenylene;
each Y⁸ and Y⁹ is independently CH or N;
s¹ is 0-3; and
** denotes attachment to the second terminus.

In some embodiments, Ring B is absent. In some embodiments, Ring B is C₄-C₇ heterocycloalkylene.

In some embodiments, Y⁸ is N. In some embodiments, Y⁸ is CH.

In some embodiments, Y⁹ is N. In some embodiments, Y⁹ is CH.

In some embodiments, L⁵ is absent.

In some embodiments, L⁵ is alkylene or alkenylene.

In some embodiments, L⁵ is —(C^{R1G}R^{1G})ₓ-(alkylene)₂-(CR^{1G}R^{1G})_{y}—; wherein x and y are each independently 0 or 1; and each R^{1G} is hydrogen or C₁-C₃ alkyl.

In some embodiments, the linker comprises a structure of Formula (C-2), or a pharmaceutically acceptable salt or solvate thereof:

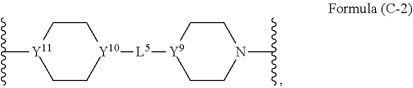

wherein
each Y¹⁰ and Y¹¹ is independently N or CH.

In some embodiments, each of Y⁸ and Y⁹ is independently N or CH; and Y⁹ is N.

In some embodiments, L⁵ is C1-C3 alkylene or C1-C3 alkenylene.

In some embodiments, L⁵ is —CH₂—, —CH₂CH₂—, —C≡C—, or —C≡C—C≡C—. In some embodiments, L⁵ is —CH₂— or —CH₂CH₂—. In some embodiments, L⁵ is —C≡C—. In some embodiments, L⁵ is —C≡C—C≡C—.

In some embodiments, the linker comprises a structure of Formula (C-3), or a pharmaceutically acceptable salt or solvate thereof:

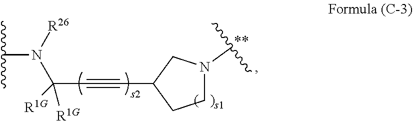

wherein,
s1 is 0-3;
s2 is 1-3;
R²⁶ is an optionally substituted C₁₋₂₀ alkylene or heteroalkylene;
each R^{IG} is independently hydrogen or C₁-C₃ alkyl; and
** denotes attachment to the second terminus.

In some embodiments, R²⁶ is an optionally substituted C₁₋₂₀ heteroalkylene. In some embodiments, R²⁶ is PEG.

In some embodiments, each R^{1G} is independently hydrogen. In some embodiments, R^{1G} is independently C₁-C₃ alkyl. In some embodiments, the C₁-C₃ alkyl is methyl, ethyl or propyl. In some embodiments, each R^{1G} is independently methyl.

In some embodiments, s1 is 0, 1, or 2. In some embodiments, s1 is 0. In some embodiments, s1 is 1. In some embodiments, s1 is 2.

In some embodiments, s2 is 1 or 2. In some embodiments, s2 is 1. In some embodiments, s2 is 2.

In some embodiments, the linker is selected from:

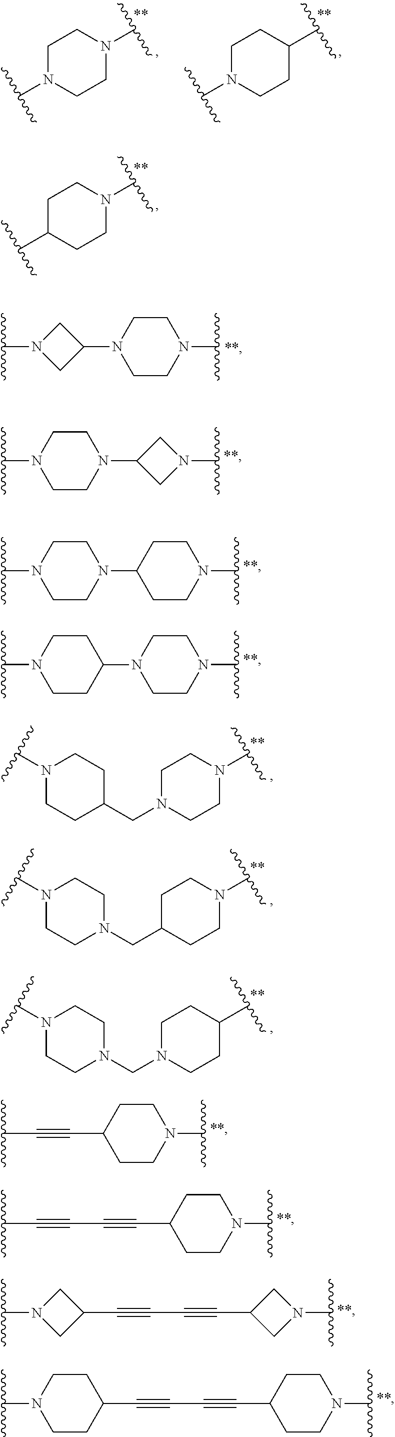

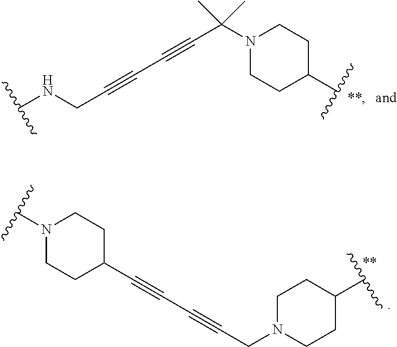 [SMILES: *NCC#CC#CC(C)(C)N1CCC(*)CC1 ; *C1CCN(CC#CC#CC2CCN(*)CC2)CC1]

In some embodiments, non-limiting examples of the transcription modulator compounds described herein are presented below in Table 3.

| | |
|---|---|
| 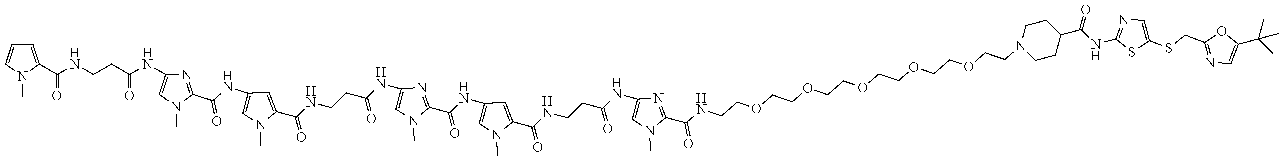 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)NCCOCCOCCOCCOCCOCCN2CCC(C(=O)Nc3ncc(SCc4ncc(C(C)(C)C)o4)s3)CC2)n1] | 1 |
| 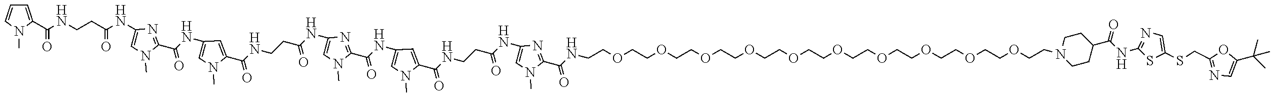 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)NCCOCCOCCOCCOCCOCCOCCOCCOCCOCCOCCN2CCC(C(=O)Nc3ncc(SCc4ncc(C(C)(C)C)o4)s3)CC2)n1] | 2 |
| 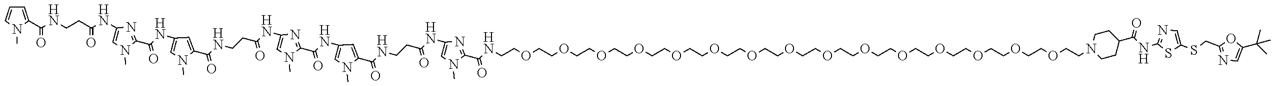 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)NCCOCCOCCOCCOCCOCCOCCOCCOCCOCCOCCOCCOCCOCCOCCOCCN2CCC(C(=O)Nc3ncc(SCc4ncc(C(C)(C)C)o4)s3)CC2)n1] | 3 |
| 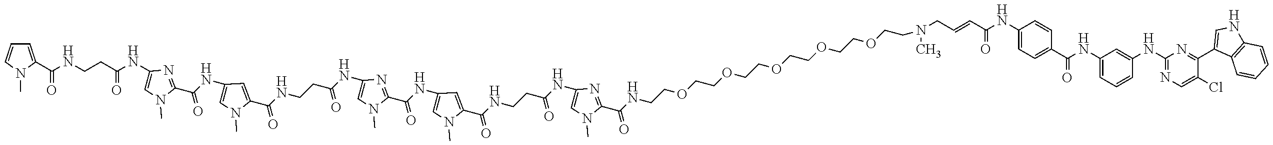 [SMILES: CN(C/C=C/C(=O)Nc1ccc(C(=O)Nc2cccc(Nc3ncc(Cl)c(-c4c[nH]c5ccccc45)n3)c2)cc1)CCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 4 |
| 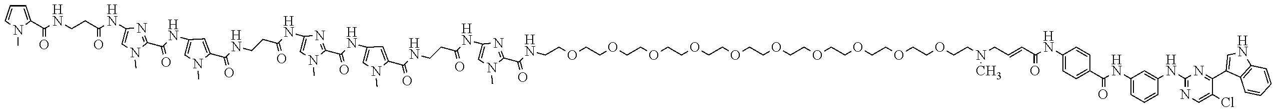 [SMILES: CN(C/C=C/C(=O)Nc1ccc(C(=O)Nc2cccc(Nc3ncc(Cl)c(-c4c[nH]c5ccccc45)n3)c2)cc1)CCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 5 |
| 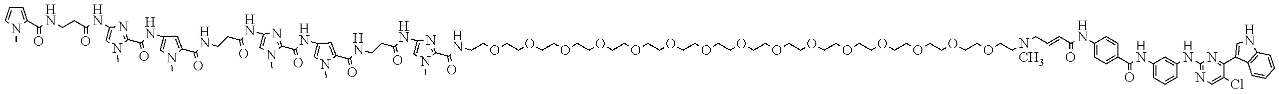 [SMILES: CN(C/C=C/C(=O)Nc1ccc(C(=O)Nc2cccc(Nc3ncc(Cl)c(-c4c[nH]c5ccccc45)n3)c2)cc1)CCOCCOCCOCCOCCOCCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 6 |
| 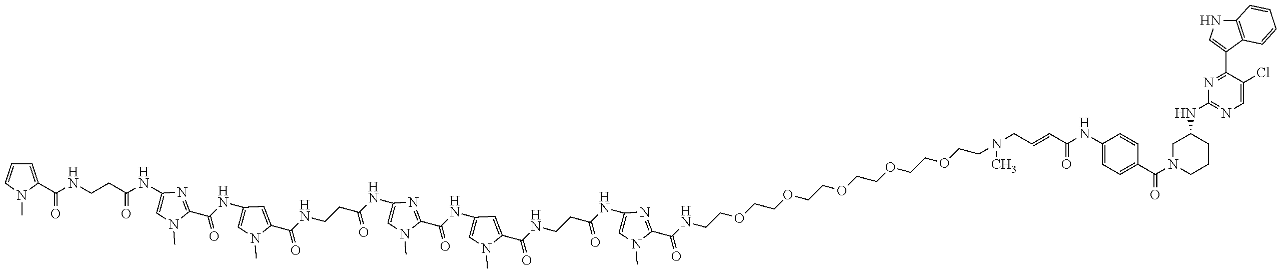 [SMILES: CN(C/C=C/C(=O)Nc1ccc(C(=O)N2CCC[C@@H](Nc3ncc(Cl)c(-c4c[nH]c5ccccc45)n3)C2)cc1)CCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 7 |
| 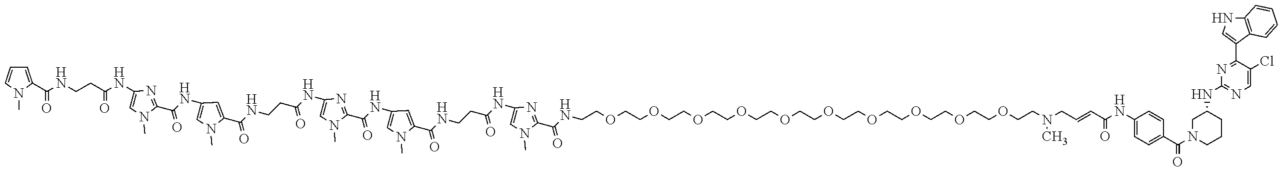 [SMILES: CN(C/C=C/C(=O)Nc1ccc(C(=O)N2CCC[C@@H](Nc3ncc(Cl)c(-c4c[nH]c5ccccc45)n3)C2)cc1)CCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 8 |
| 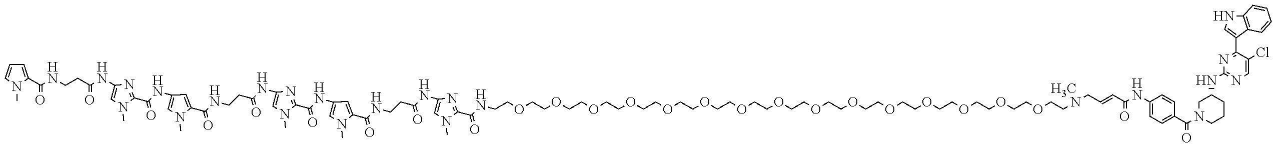 [SMILES: CN(C/C=C/C(=O)Nc1ccc(C(=O)N2CCC[C@@H](Nc3ncc(Cl)c(-c4c[nH]c5ccccc45)n3)C2)cc1)CCOCCOCCOCCOCCOCCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 9 |
| 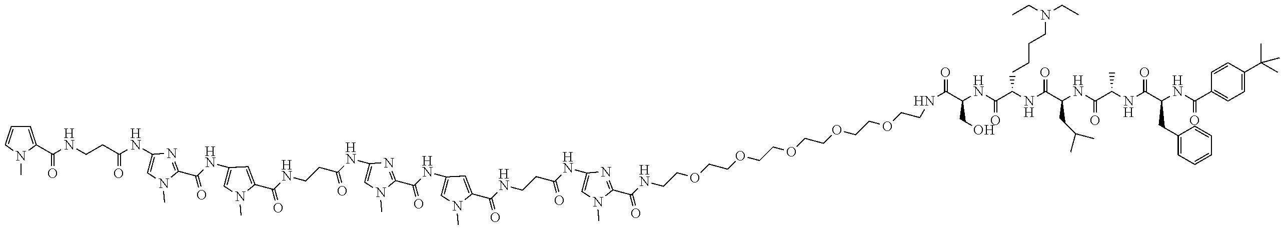 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 10 |
| 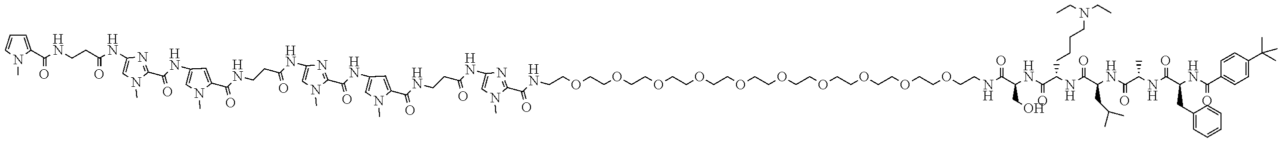 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 11 |
| 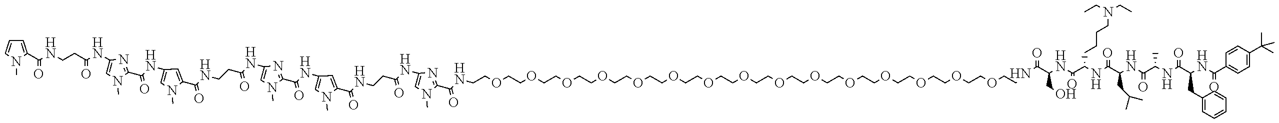 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 12 |
| 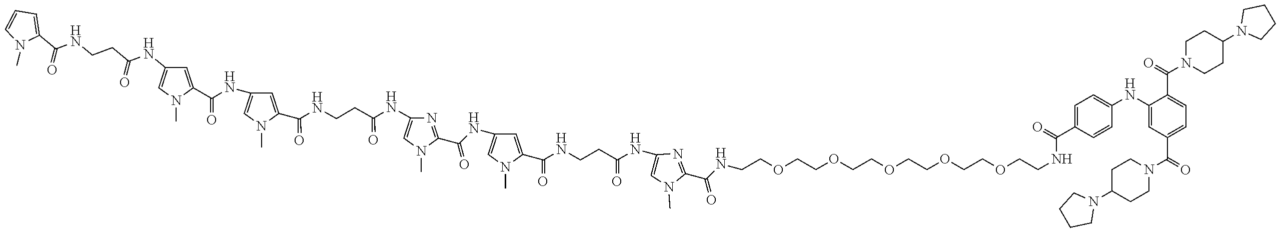 [SMILES: Cn1cccc1C(=O)NCCC(=O)Nc1cc(C(=O)Nc2cc(C(=O)NCCC(=O)Nc3cn(C)c(C(=O)Nc4cc(C(=O)NCCC(=O)Nc5cn(C)c(C(=O)NCCOCCOCCOCCOCCOCCNC(=O)c6ccc(Nc7cc(C(=O)N8CCC(N9CCCC9)CC8)ccc7C(=O)N7CCC(N8CCCC8)CC7)cc6)n5)n(C)c4)n3)n(C)c2)n(C)c1] | 13 |
| 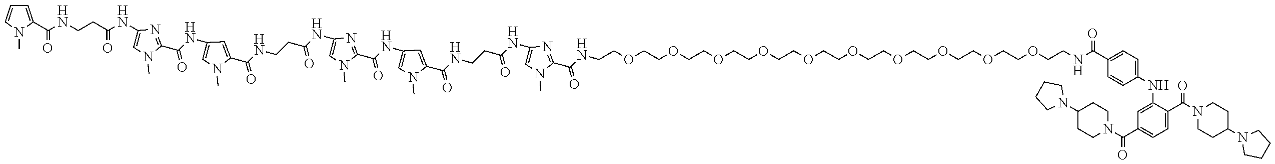 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)NCCOCCOCCOCCOCCOCCOCCOCCOCCOCCOCCNC(=O)c2ccc(Nc3cc(C(=O)N4CCC(N5CCCC5)CC4)ccc3C(=O)N3CCC(N4CCCC4)CC3)cc2)n1] | 14 |
| 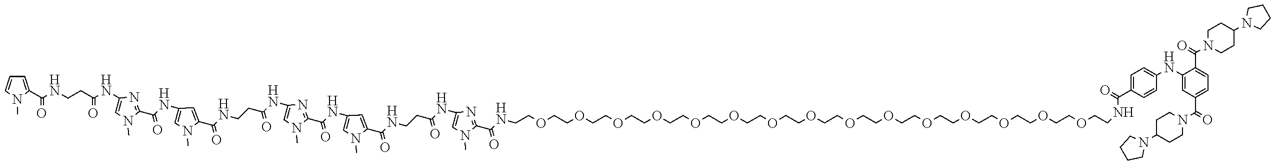 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)NCCOCCOCCOCCOCCOCCOCCOCCOCCOCCOCCOCCOCCOCCOCCOCCNC(=O)c2ccc(Nc3cc(C(=O)N4CCC(N5CCCC5)CC4)ccc3C(=O)N3CCC(N4CCCC4)CC3)cc2)n1] | 15 |
| 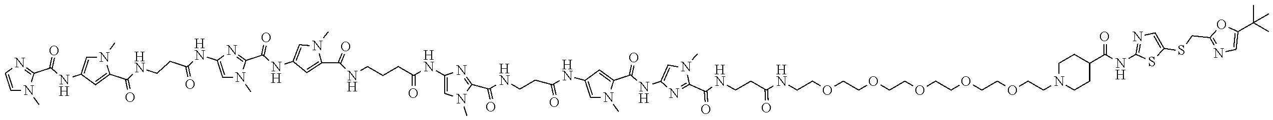 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCN4CCC(C(=O)Nc5ncc(SCc6ncc(C(C)(C)C)o6)s5)CC4)n3)n(C)c2)n1] | 16 |
| 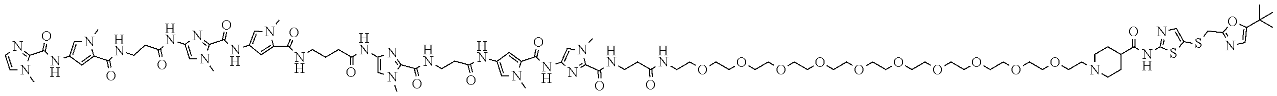 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOCCOCCOCCOCCOCCN4CCC(C(=O)Nc5ncc(SCc6ncc(C(C)(C)C)o6)s5)CC4)n3)n(C)c2)n1] | 17 |
| 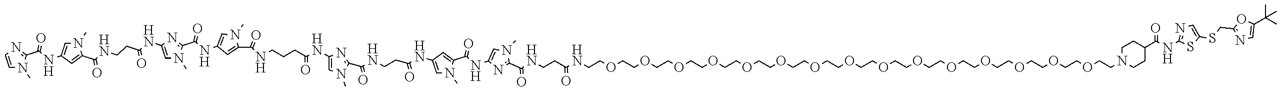 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOCCOCCOCCOCCOCCOCCOCCOCCOCCOCCN4CCC(C(=O)Nc5ncc(SCc6ncc(C(C)(C)C)o6)s5)CC4)n3)n(C)c2)n1] | 18 |
| 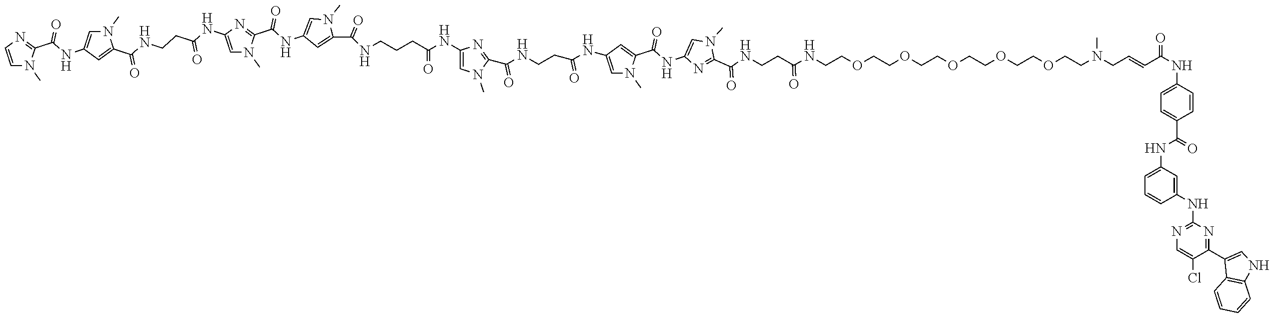 [SMILES: CN(C/C=C/C(=O)Nc1ccc(C(=O)Nc2cccc(Nc3ncc(Cl)c(-c4c[nH]c5ccccc45)n3)c2)cc1)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 19 |
| 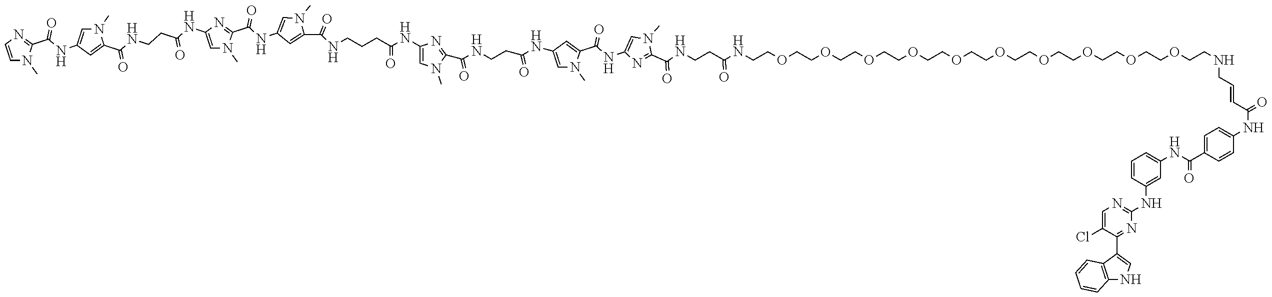 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOCCOCCOCCOCCOCCNC/C=C/C(=O)Nc4ccc(C(=O)Nc5cccc(Nc6ncc(Cl)c(-c7c[nH]c8ccccc78)n6)c5)cc4)n3)n(C)c2)n1] | 20 |
| 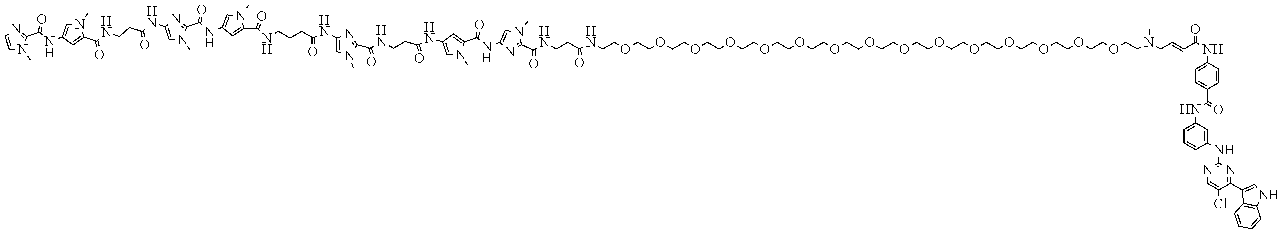 [SMILES: CN(C/C=C/C(=O)Nc1ccc(C(=O)Nc2cccc(Nc3ncc(Cl)c(-c4c[nH]c5ccccc45)n3)c2)cc1)CCOCCOCCOCCOCCOCCOCCOCCOCCOCCOCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 21 |
| 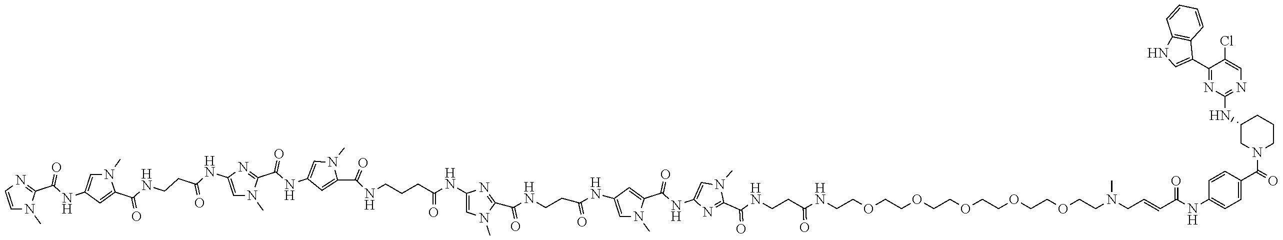 [SMILES: CN(C/C=C/C(=O)Nc1ccc(C(=O)N2CCC[C@@H](Nc3ncc(Cl)c(-c4c[nH]c5ccccc45)n3)C2)cc1)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 22 |
| 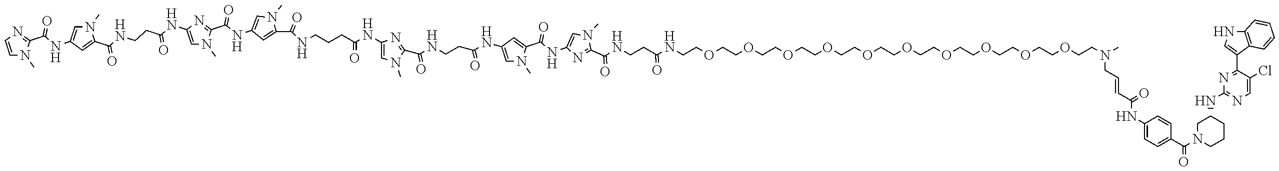 [SMILES: CN(C/C=C/C(=O)Nc1ccc(C(=O)N2CCC[C@@H](Nc3ncc(Cl)c(-c4c[nH]c5ccccc45)n3)C2)cc1)CCOCCOCCOCCOCCOCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 23 |
| 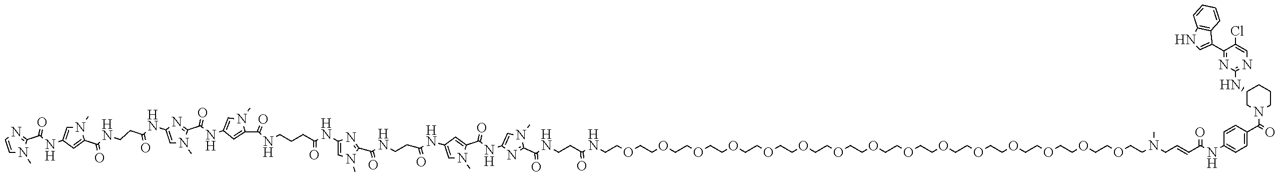 [SMILES: CN(C/C=C/C(=O)Nc1ccc(C(=O)N2CCC[C@@H](Nc3ncc(Cl)c(-c4c[nH]c5ccccc45)n3)C2)cc1)CCOCCOCCOCCOCCOCCOCCOCCOCCOCCOCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 24 |
| 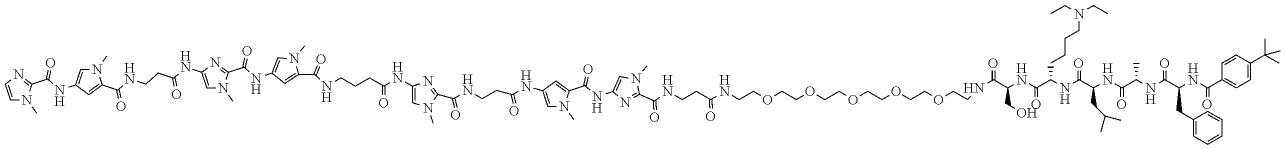 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 25 |
| 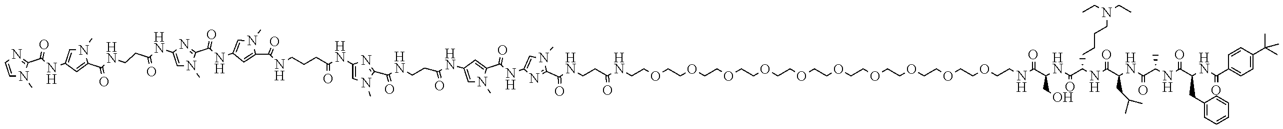 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 26 |
| 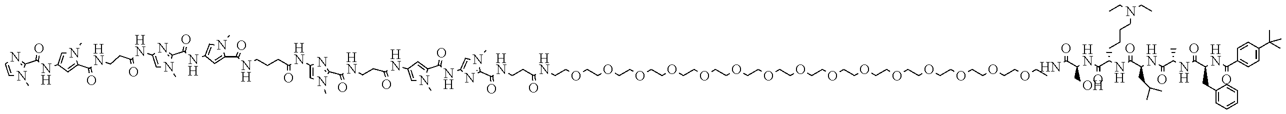 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCOCCOCCOCCOCCOCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 27 |
| 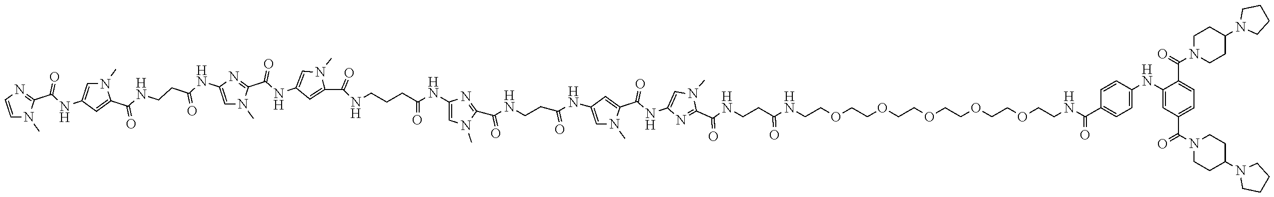 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCNC(=O)c4ccc(Nc5cc(C(=O)N6CCC(N7CCCC7)CC6)ccc5C(=O)N5CCC(N6CCCC6)CC5)cc4)n3)n(C)c2)n1] | 28 |
| 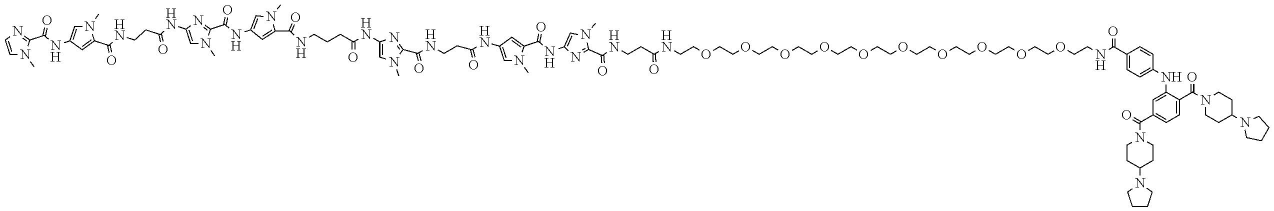 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOCCOCCOCCOCCOCCNC(=O)c4ccc(Nc5cc(C(=O)N6CCC(N7CCCC7)CC6)ccc5C(=O)N5CCC(N6CCCC6)CC5)cc4)n3)n(C)c2)n1] | 29 |
| 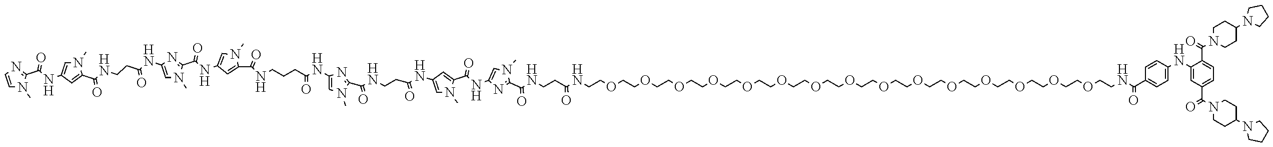 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOCCOCCOCCOCCOCCOCCOCCOCCOCCOCCNC(=O)c4ccc(Nc5cc(C(=O)N6CCC(N7CCCC7)CC6)ccc5C(=O)N5CCC(N6CCCC6)CC5)cc4)n3)n(C)c2)n1] | 30 |
| 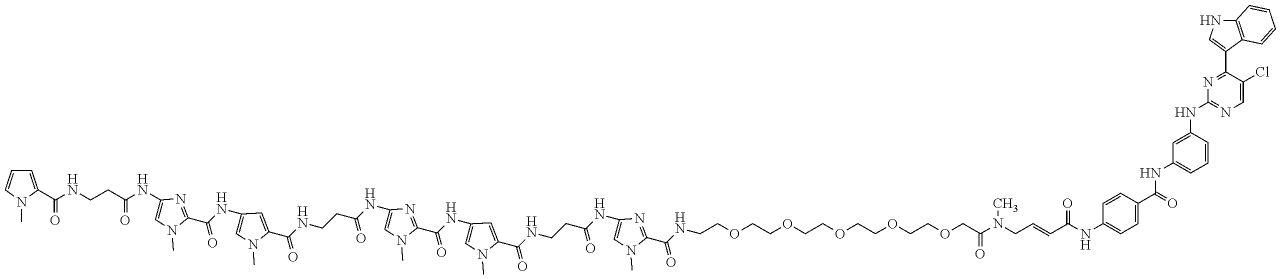 [SMILES: CN(C/C=C/C(=O)Nc1ccc(C(=O)Nc2cccc(Nc3ncc(Cl)c(-c4c[nH]c5ccccc45)n3)c2)cc1)C(=O)COCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 31 |
| 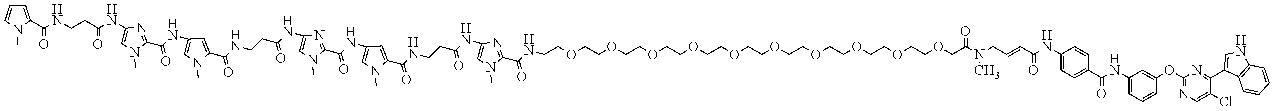 [SMILES: CN(C/C=C/C(=O)Nc1ccc(C(=O)Nc2cccc(Oc3ncc(Cl)c(-c4c[nH]c5ccccc45)n3)c2)cc1)C(=O)COCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 32 |
| 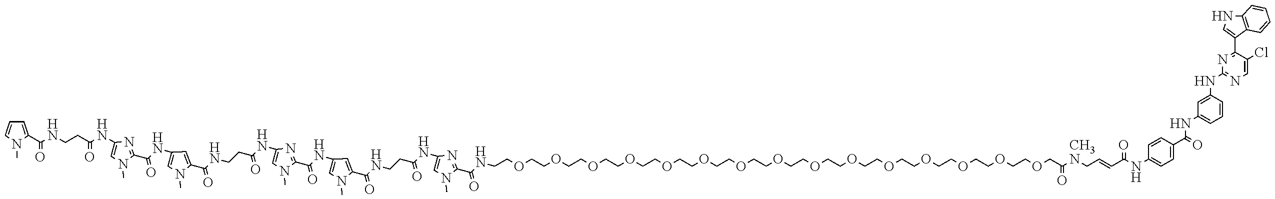 [SMILES: CN(C/C=C/C(=O)Nc1ccc(C(=O)Nc2cccc(Nc3ncc(Cl)c(-c4c[nH]c5ccccc45)n3)c2)cc1)C(=O)COCCOCCOCCOCCOCCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 33 |
| 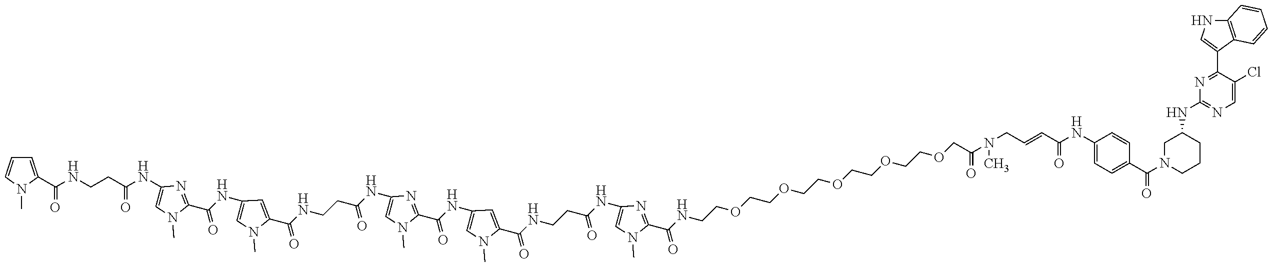 [SMILES: CN(C/C=C/C(=O)Nc1ccc(C(=O)N2CCC[C@@H](Nc3ncc(Cl)c(-c4c[nH]c5ccccc45)n3)C2)cc1)C(=O)COCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 34 |
| 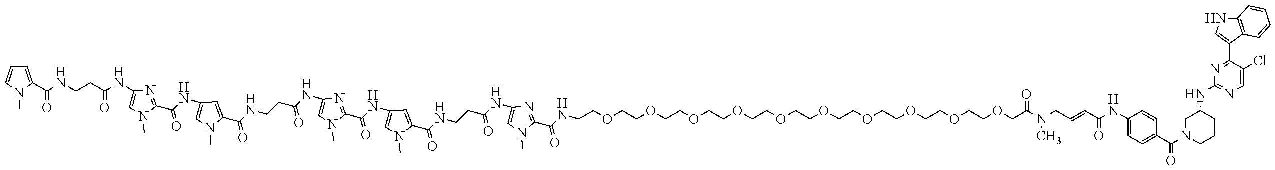 [SMILES: CN(C/C=C/C(=O)Nc1ccc(C(=O)N2CCC[C@@H](Nc3ncc(Cl)c(-c4c[nH]c5ccccc45)n3)C2)cc1)C(=O)COCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 35 |
| 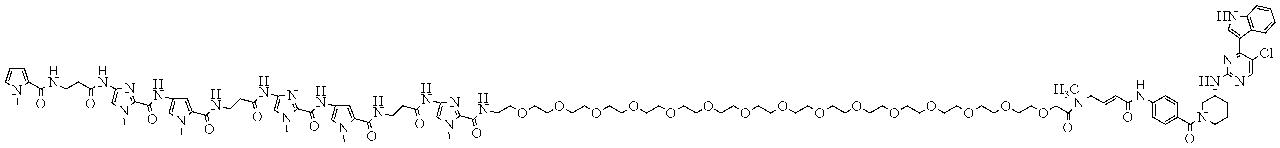 [SMILES: CN(C/C=C/C(=O)Nc1ccc(C(=O)N2CCC[C@@H](Nc3ncc(Cl)c(-c4c[nH]c5ccccc45)n3)C2)cc1)C(=O)COCCOCCOCCOCCOCCOCCOCCOCCOCCOCCOCCOCCOCCOCCOCCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C] | 36 |
| 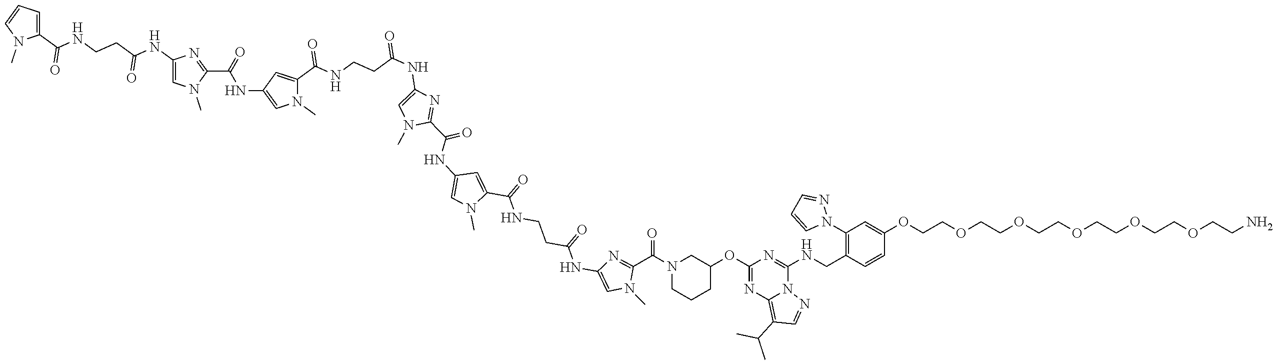 [SMILES: CC(C)c1cnn2c(NCc3ccc(OCCOCCOCCOCCOCCOCCN)cc3-n3cccn3)nc(OC3CCCN(C(=O)c4nc(NC(=O)CCNC(=O)c5cc(NC(=O)c6nc(NC(=O)CCNC(=O)c7cc(NC(=O)c8nc(NC(=O)CCNC(=O)c9cccn9C)cn8C)cn7C)cn6C)cn5C)cn4C)C3)nc12] | 37 |
| 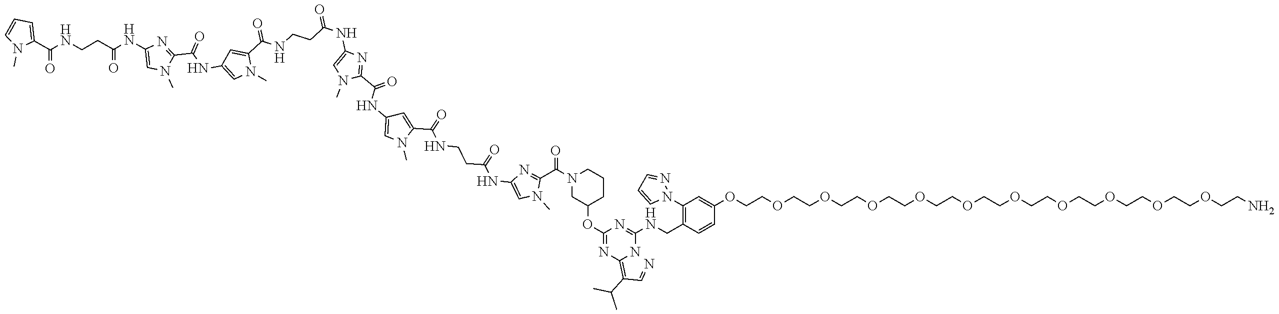 [SMILES: CC(C)c1cnn2c(NCc3ccc(OCCOCCOCCOCCOCCOCCOCCOCCOCCOCCOCCN)cc3-n3cccn3)nc(OC3CCCN(C(=O)c4nc(NC(=O)CCNC(=O)c5cc(NC(=O)c6nc(NC(=O)CCNC(=O)c7cc(NC(=O)c8nc(NC(=O)CCNC(=O)c9cccn9C)cn8C)cn7C)cn6C)cn5C)cn4C)C3)nc12] | 38 |
| 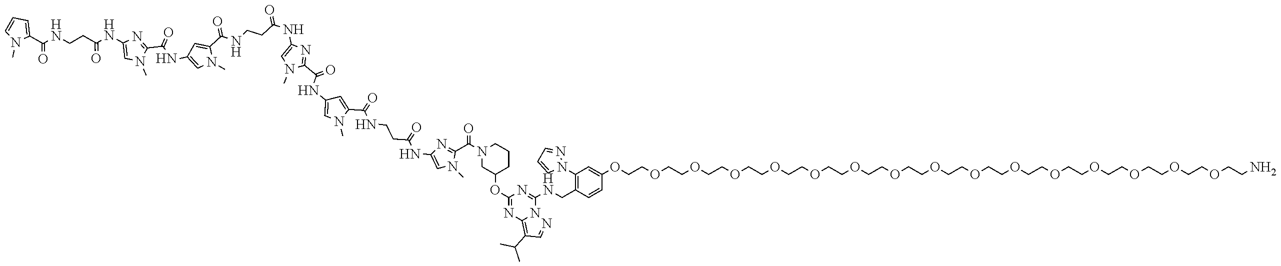 [SMILES: CC(C)c1cnn2c(NCc3ccc(OCCOCCOCCOCCOCCOCCOCCOCCOCCOCCOCCOCCOCCOCCOCCOCCN)cc3-n3cccn3)nc(OC3CCCN(C(=O)c4nc(NC(=O)CCNC(=O)c5cc(NC(=O)c6nc(NC(=O)CCNC(=O)c7cc(NC(=O)c8nc(NC(=O)CCNC(=O)c9cccn9C)cn8C)cn7C)cn6C)cn5C)cn4C)C3)nc12] | 39 |
| 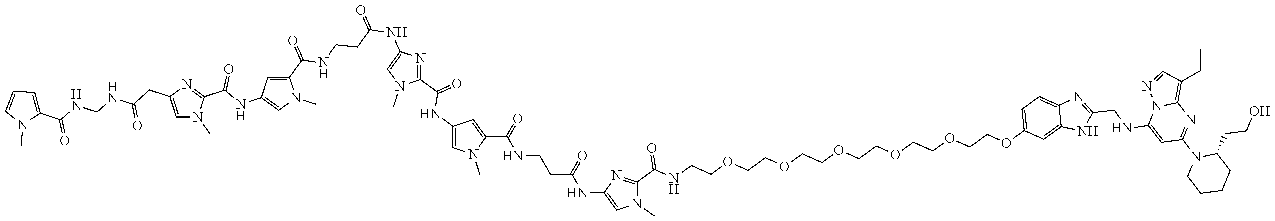 [SMILES: CCc1cnn2c(NCc3nc4ccc(OCCOCCOCCOCCOCCOCCNC(=O)c5nc(NC(=O)CCNC(=O)c6cc(NC(=O)c7nc(NC(=O)CCNC(=O)c8cc(NC(=O)c9nc(CC(=O)NCNC(=O)c%10cccn%10C)cn9C)cn8C)cn7C)cn6C)cn5C)cc4[nH]3)cc(N3CCCC[C@H]3CCO)nc12] | 40 |
| 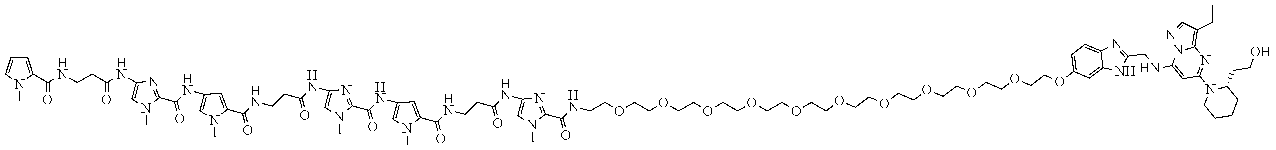 [SMILES: CCc1cnn2c(NCc3nc4ccc(OCCOCCOCCOCCOCCOCCOCCOCCOCCOCCOCCNC(=O)c5nc(NC(=O)CCNC(=O)c6cc(NC(=O)c7nc(NC(=O)CCNC(=O)c8cc(NC(=O)c9nc(NC(=O)CCNC(=O)c%10cccn%10C)cn9C)cn8C)cn7C)cn6C)cn5C)cc4[nH]3)cc(N3CCCC[C@H]3CCO)nc12] | 41 |
| 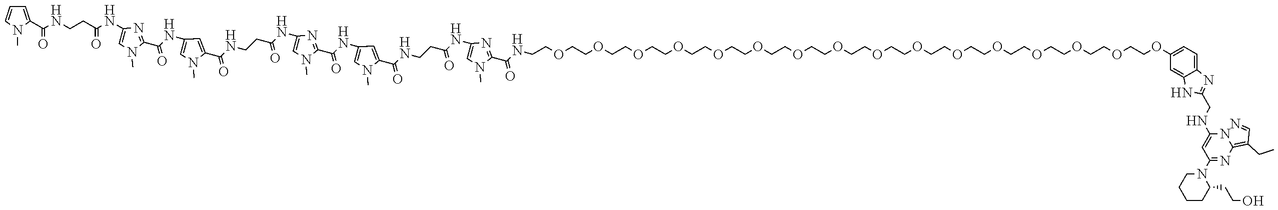 [SMILES: CC/C(C=N)=C1\N=C(N2CCCC[C@H]2CCO)C=C(NCc2nc3ccc(OCCOCCOCCOCCOCCOCCOCCOCCOCCOCCOCCOCCOCCOCCOCCOCCNC(=O)c4nc(NC(=O)CCNC(=O)c5cc(NC(=O)c6nc(NC(=O)CCNC(=O)c7cc(NC(=O)c8nc(NC(=O)CCNC(=O)c9cccn9C)cn8C)cn7C)cn6C)cn5C)cn4C)cc3[nH]2)N1] | 42 |
| 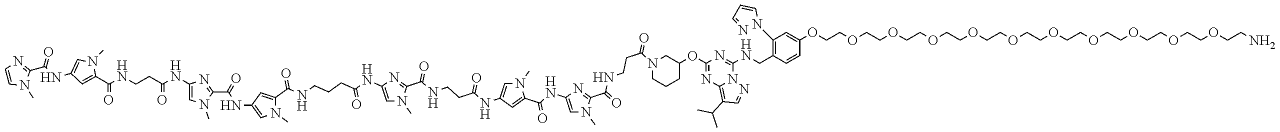 [SMILES: CC(C)c1cnn2c(NCc3ccc(OCCOCCOCCOCCOCCOCCOCCOCCOCCOCCOCCN)cc3-n3cccn3)nc(OC3CCCN(C(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)C3)nc12] | 43 |
| 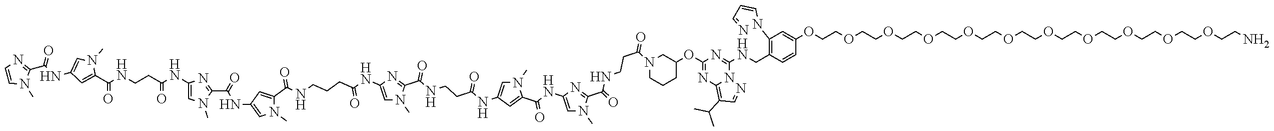 [SMILES: CC(C)c1cnn2c(NCc3ccc(OCCOCCOCCOCCOCCOCCOCCOCCOCCOCCOCCN)cc3-n3cccn3)nc(OC3CCCN(C(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)C3)nc12] | 44 |
| 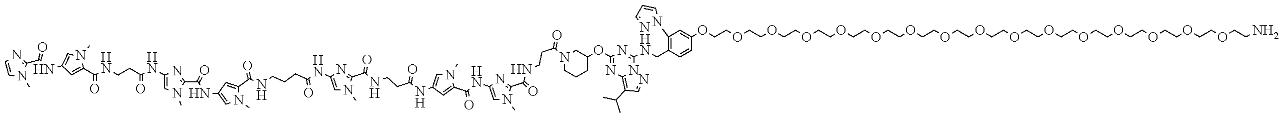 [SMILES: CC(C)c1cnn2c(NCc3ccc(OCCOCCOCCOCCOCCOCCOCCOCCOCCOCCOCCOCCOCCOCCOCCOCCN)cc3-n3cccn3)nc(OC3CCCN(C(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)C3)nc12] | 45 |
| 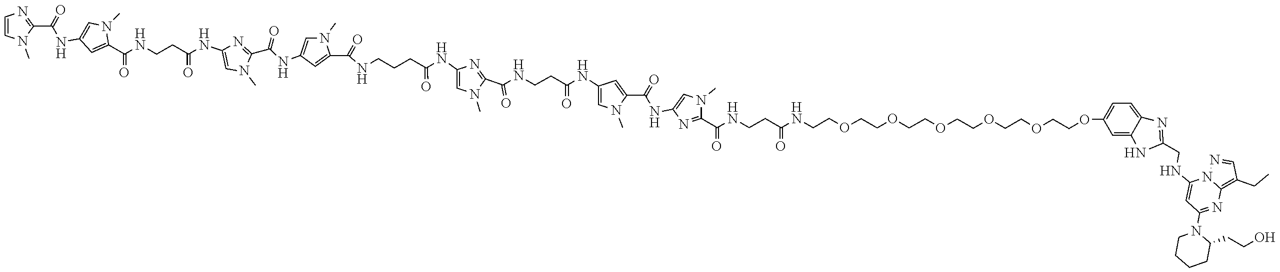 [SMILES: CCc1cnn2c(NCc3nc4ccc(OCCOCCOCCOCCOCCOCCNC(=O)CCNC(=O)c5nc(NC(=O)c6cc(NC(=O)CCNC(=O)c7nc(NC(=O)CCCNC(=O)c8cc(NC(=O)c9nc(NC(=O)CCNC(=O)c%10cc(NC(=O)c%11nccn%11C)cn%10C)cn9C)cn8C)cn7C)cn6C)cn5C)cc4[nH]3)cc(N3CCCC[C@H]3CCO)nc12] | 46 |
| 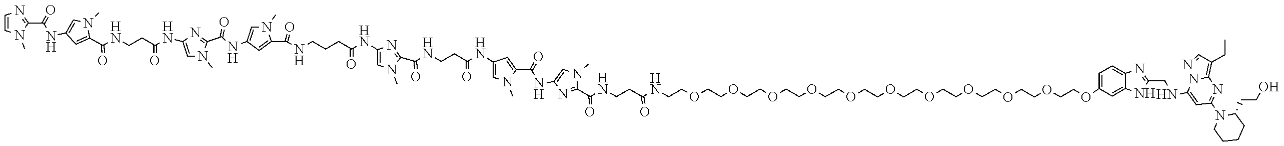 [SMILES: CCc1cnn2c(NCc3nc4ccc(OCCOCCOCCOCCOCCOCCOCCOCCOCCOCCOCCNC(=O)CCNC(=O)c5nc(NC(=O)c6cc(NC(=O)CCNC(=O)c7nc(NC(=O)CCCNC(=O)c8cc(NC(=O)c9nc(NC(=O)CCNC(=O)c%10cc(NC(=O)c%11nccn%11C)cn%10C)cn9C)cn8C)cn7C)cn6C)cn5C)cc4[nH]3)cc(N3CCCC[C@H]3CCO)nc12] | 47 |
| 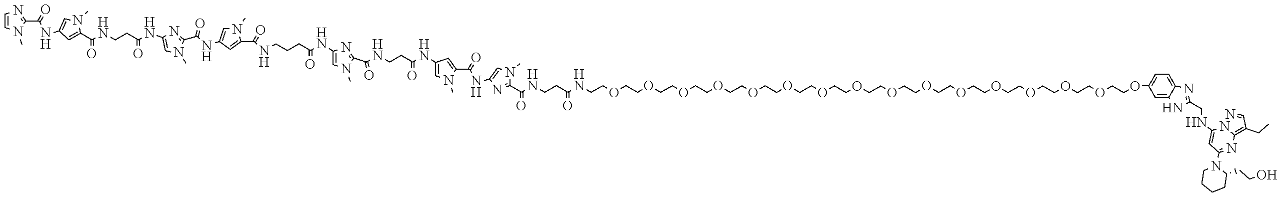 [SMILES: CCc1cnn2c(NCc3nc4ccc(OCCOCCOCCOCCOCCOCCOCCOCCOCCOCCOCCOCCOCCOCCOCCOCCNC(=O)CCNC(=O)c5nc(NC(=O)c6cc(NC(=O)CCNC(=O)c7nc(NC(=O)CCCNC(=O)c8cc(NC(=O)c9nc(NC(=O)CCNC(=O)c%10cc(NC(=O)c%11nccn%11C)cn%10C)cn9C)cn8C)cn7C)cn6C)cn5C)cc4[nH]3)cc(N3CCCC[C@H]3CCO)nc12] | 48 |
| 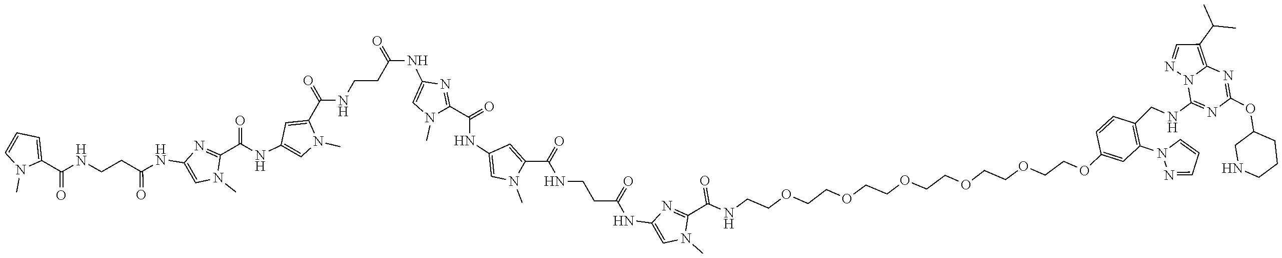 [SMILES: CC(C)c1cnn2c(NCc3ccc(OCCOCCOCCOCCOCCOCCNC(=O)c4nc(NC(=O)CCNC(=O)c5cc(NC(=O)c6nc(NC(=O)CCNC(=O)c7cc(NC(=O)c8nc(NC(=O)CCNC(=O)c9cccn9C)cn8C)cn7C)cn6C)cn5C)cn4C)cc3-n3cccn3)nc(OC3CCCNC3)nc12] | 49 |
| 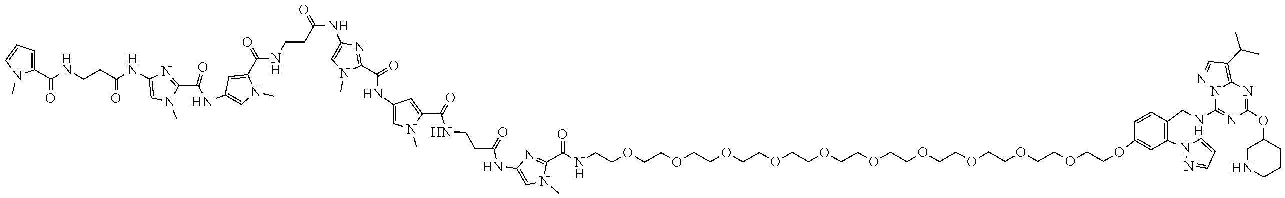 [SMILES: CC(C)c1cnn2c(NCc3ccc(OCCOCCOCCOCCOCCOCCOCCOCCOCCOCCOCCNC(=O)c4nc(NC(=O)CCNC(=O)c5cc(NC(=O)c6nc(NC(=O)CCNC(=O)c7cc(NC(=O)c8nc(NC(=O)CCNC(=O)c9cccn9C)cn8C)cn7C)cn6C)cn5C)cn4C)cc3-n3cccn3)nc(OC3CCCNC3)nc12] | 50 |
| 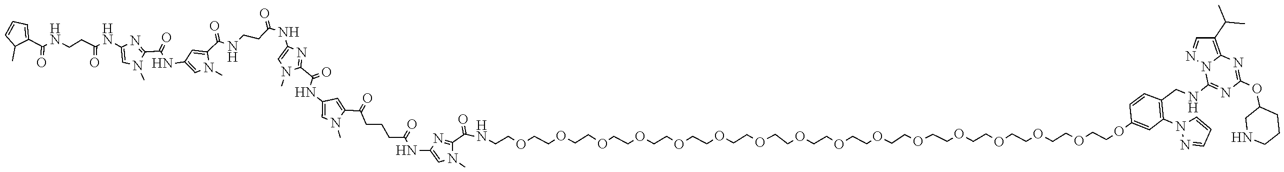 | 51 |
| 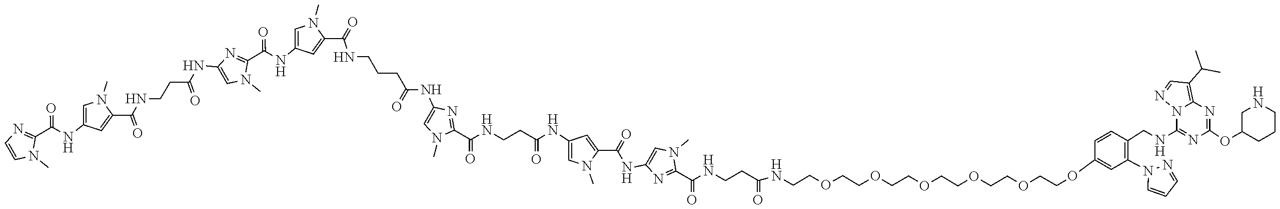 [SMILES: CC(C)c1cnn2c(NCc3ccc(OCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)cc3-n3cccn3)nc(OC3CCCNC3)nc12] | 52 |
| 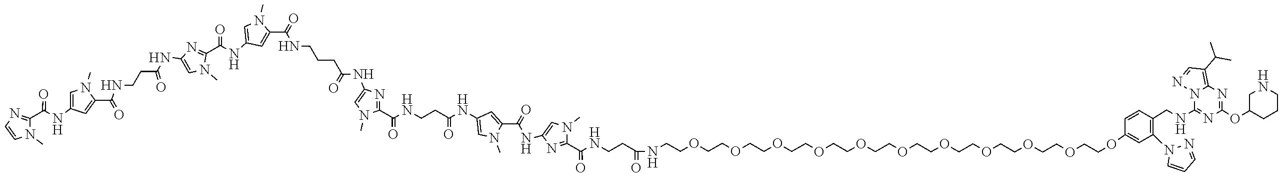 [SMILES: CC(C)c1cnn2c(NCc3ccc(OCCOCCOCCOCCOCCOCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)cc3-n3cccn3)nc(OC3CCCNC3)nc12] | 53 |
| 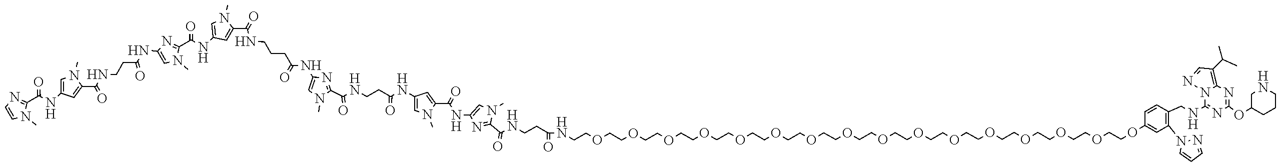 [SMILES: CC(C)c1cnn2c(NCc3ccc(OCCOCCOCCOCCOCCOCCOCCOCCOCCOCCOCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)cc3-n3cccn3)nc(OC3CCCNC3)nc12] | 54 |
| 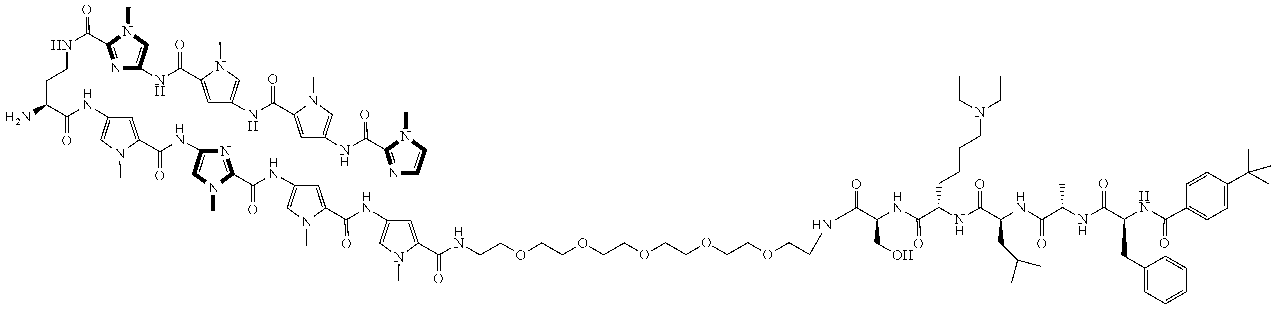 | 55 |
| 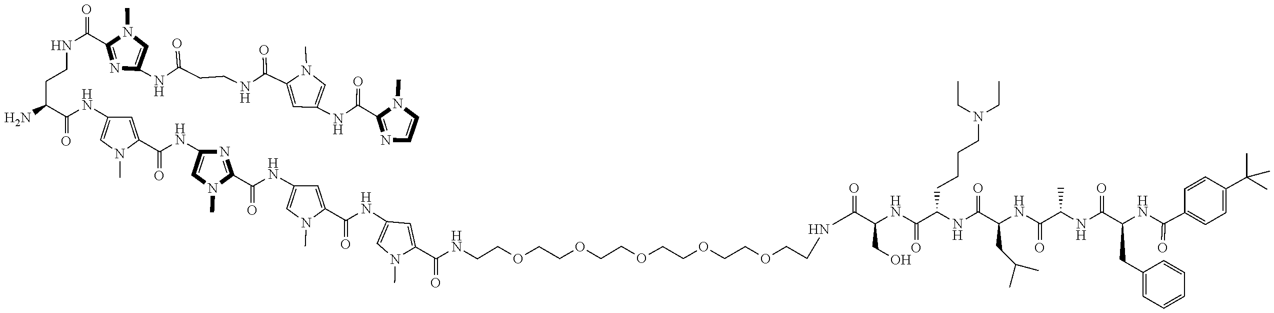 | 56 |
| 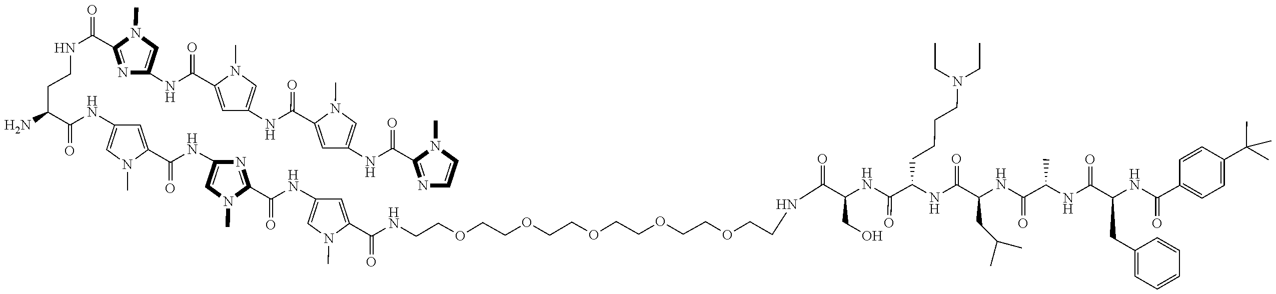 | 57 |
| 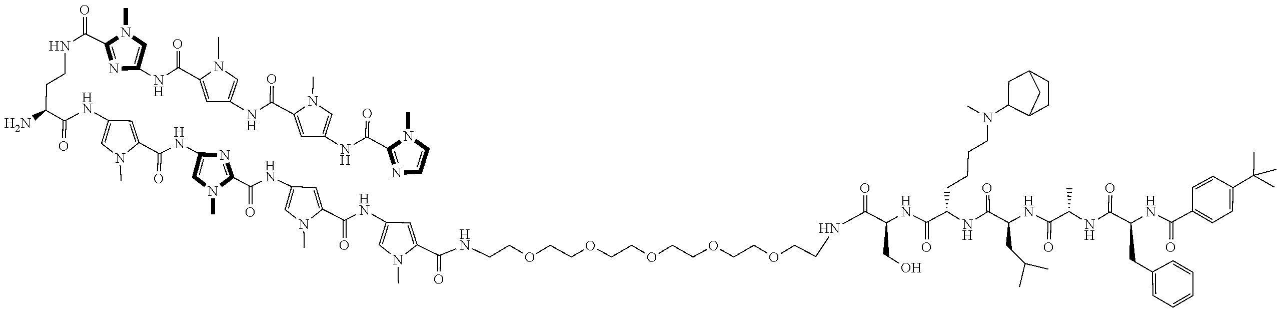 | 58 |
| 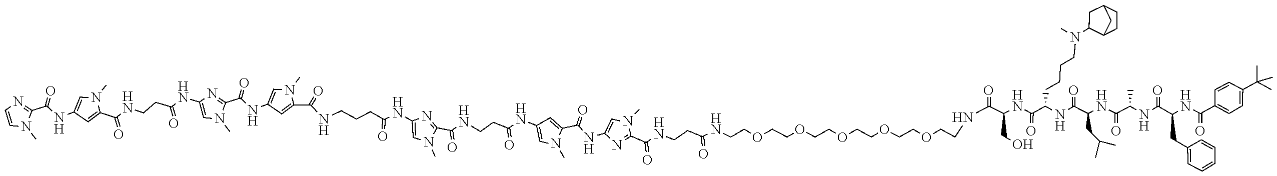 [SMILES: CC(C)C[C@H](NC(=O)[C@H](C)NC(=O)[C@H](Cc1ccccc1)NC(=O)c1ccc(C(C)(C)C)cc1)C(=O)N[C@@H](CCCCN(C)C1CC2CCC1C2)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 59 |
| 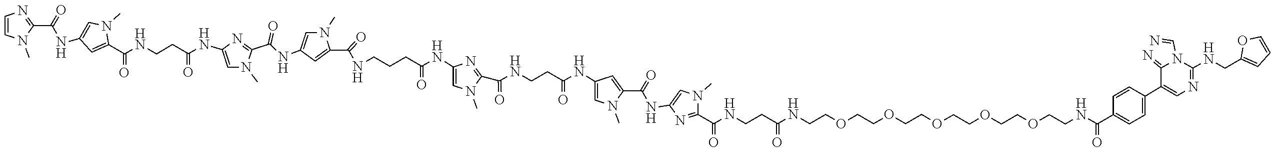 | 60 |
| 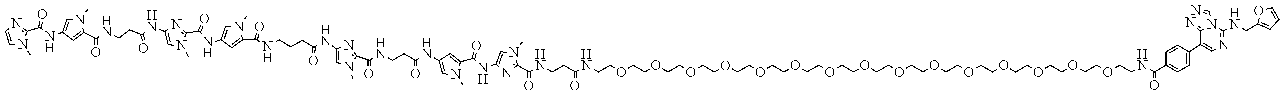 | 61 |
| 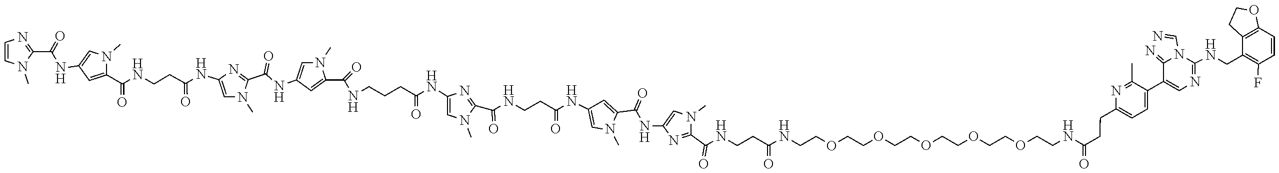 | 62 |
| 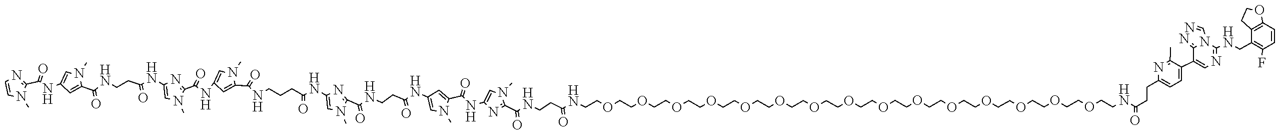 | 63 |
| 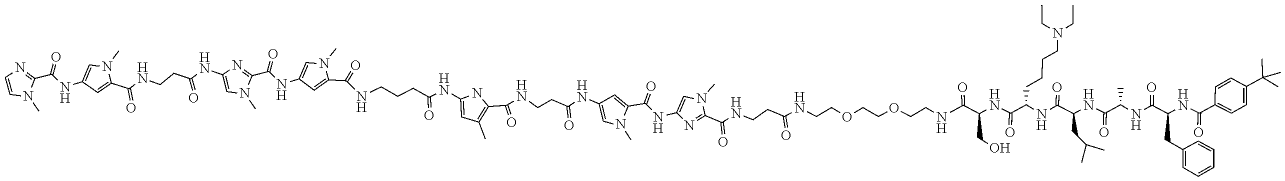 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCNC(=O)CCNC(=O)c1nc(NC(=O)c2cc(NC(=O)CCNC(=O)c3[nH]c(NC(=O)CCCNC(=O)c4cc(NC(=O)c5nc(NC(=O)CCNC(=O)c6cc(NC(=O)c7nccn7C)cn6C)cn5C)cn4C)cc3C)cn2C)cn1C] | 64 |
| 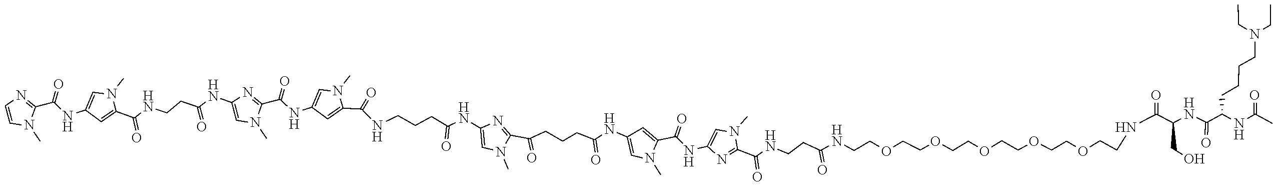 [SMILES: CCN(CC)CCCC[C@H](NC(C)=O)C(=O)N[C@@H](CO)C(=O)NCCOCCOCCOCCOCCOCCNC(=O)CCNC(=O)c1nc(NC(=O)c2cc(NC(=O)CCCC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 65 |
| 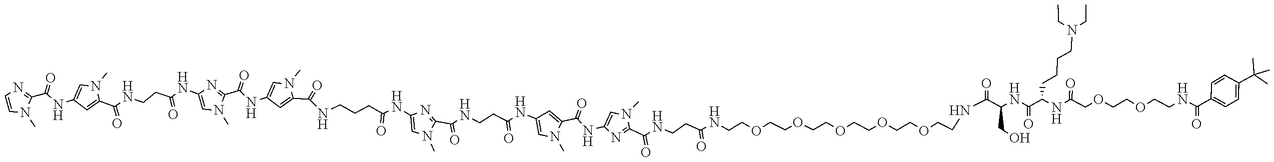 [SMILES: CCN(CC)CCCC[C@H](NC(=O)COCCOCCNC(=O)c1ccc(C(C)(C)C)cc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 66 |
| 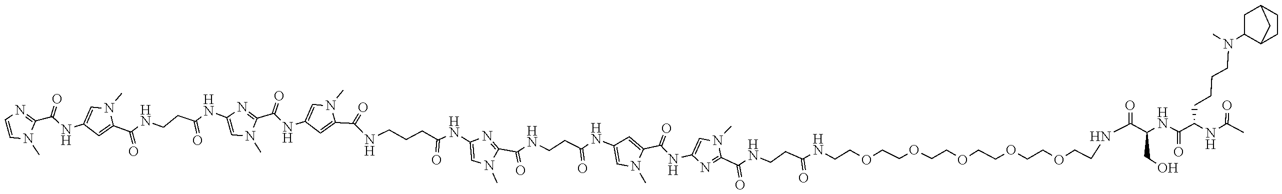 [SMILES: CC(=O)N[C@@H](CCCCN(C)C1CC2CCC1C2)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 67 |
| 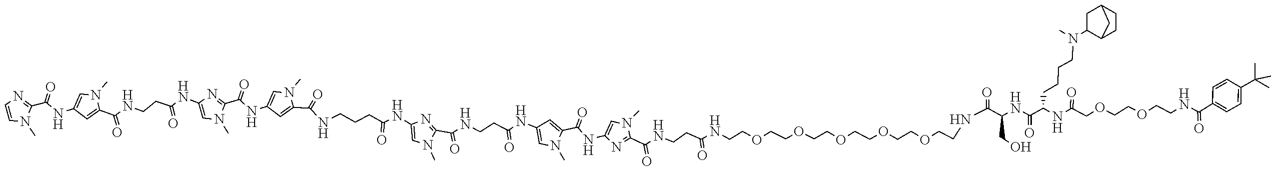 [SMILES: CN(CCCC[C@H](NC(=O)COCCOCCNC(=O)c1ccc(C(C)(C)C)cc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)C1CC2CCC1C2] | 68 |
| 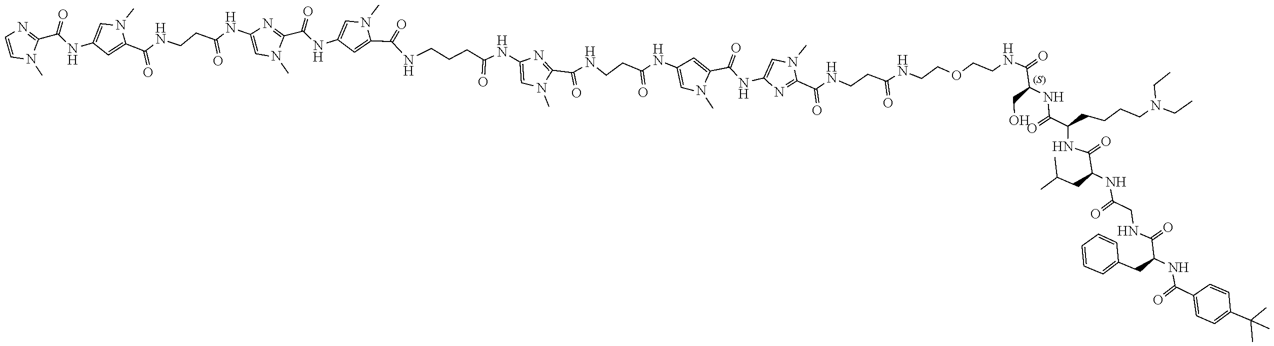 | 69 |
| 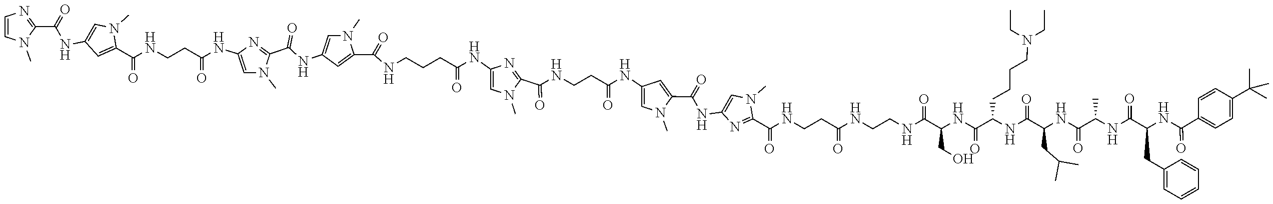 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 70 |
| 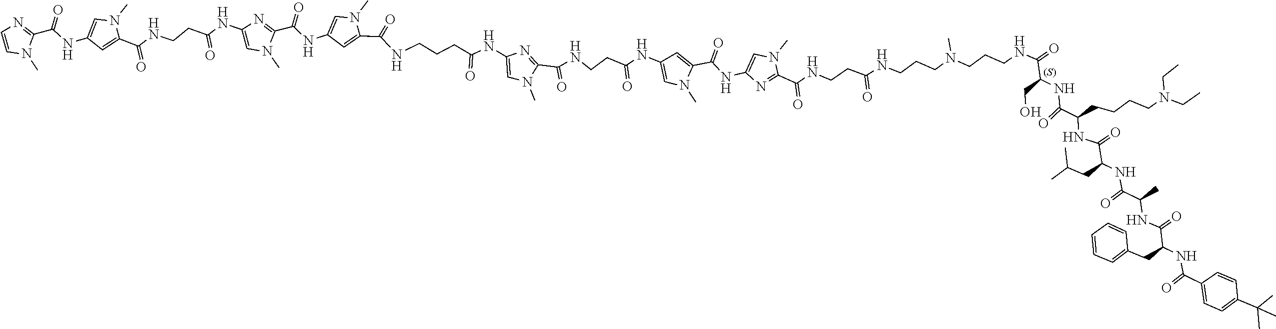 | 71 |
| 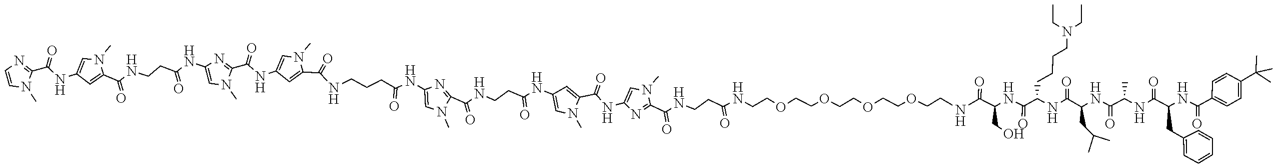 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 72 |
| 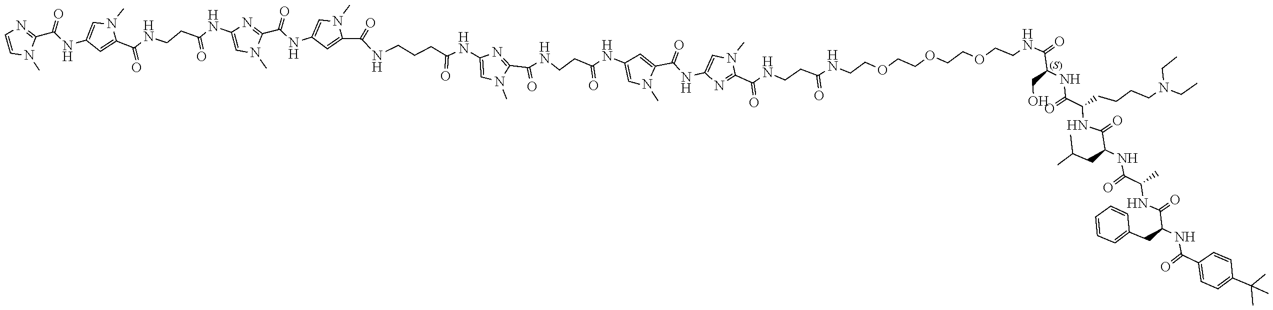 | 73 |
| 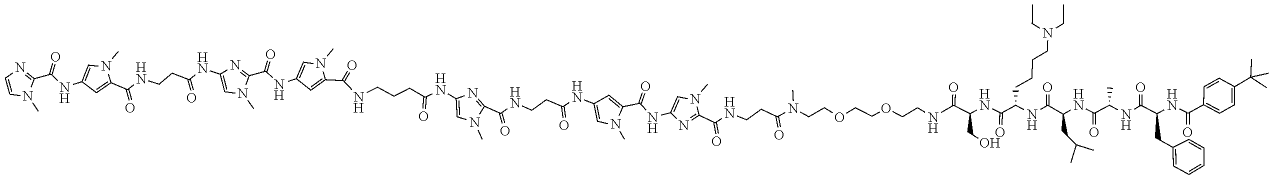 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCN(C)C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 74 |
| 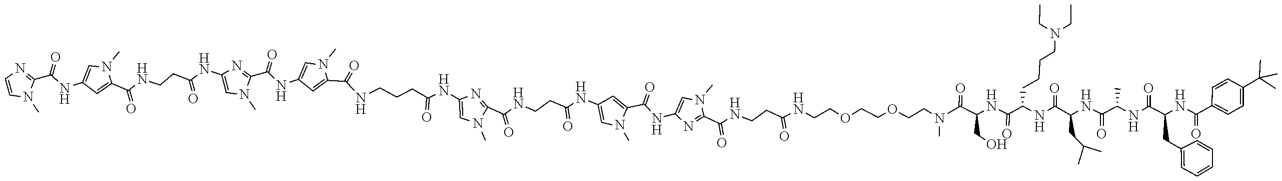 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)N(C)CCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 75 |
| 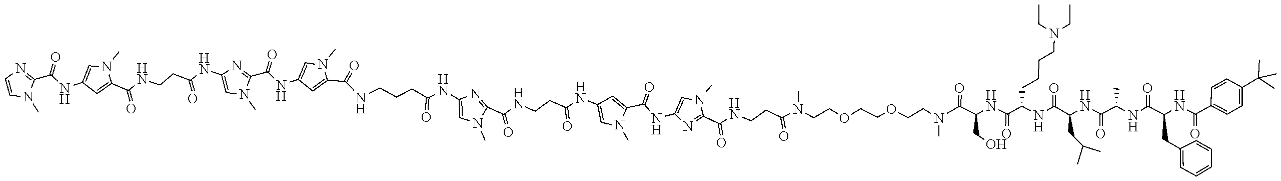 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)N(C)CCOCCOCCN(C)C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 76 |
| 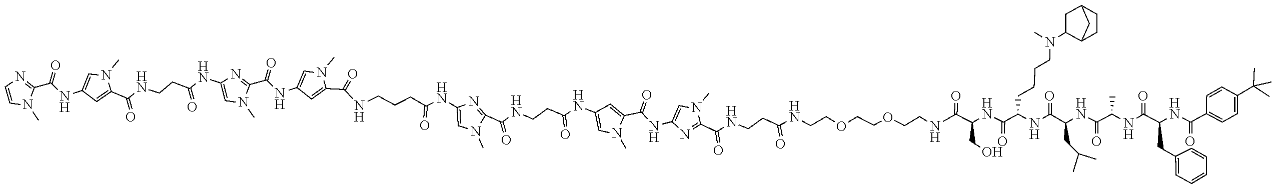 [SMILES: CC(C)C[C@H](NC(=O)[C@H](C)NC(=O)[C@H](Cc1ccccc1)NC(=O)c1ccc(C(C)(C)C)cc1)C(=O)N[C@@H](CCCCN(C)C1CC2CCC1C2)C(=O)N[C@@H](CO)C(=O)NCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 77 |
| 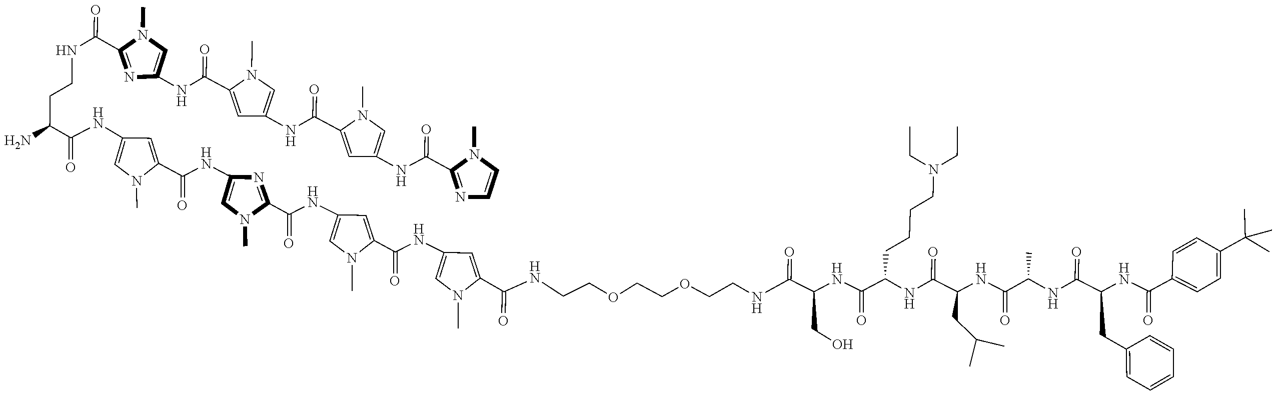 | 78 |
| 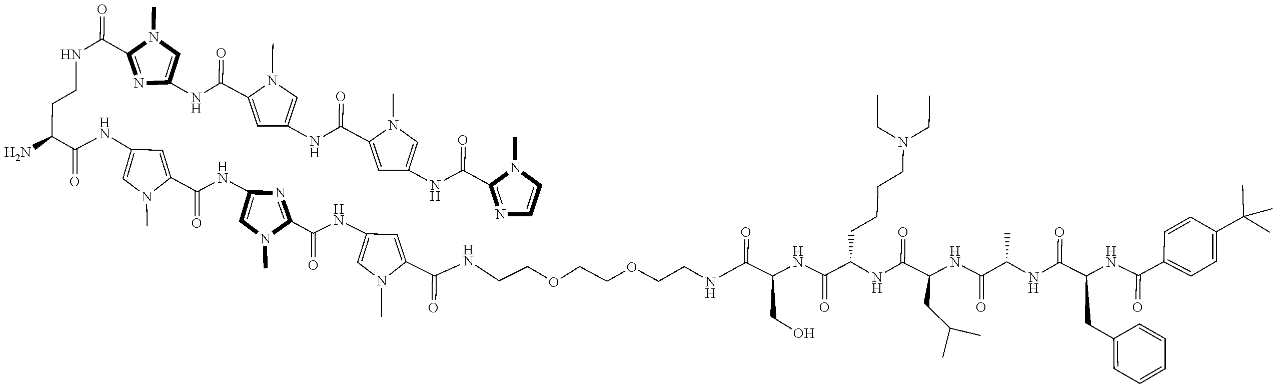 | 79 |
| 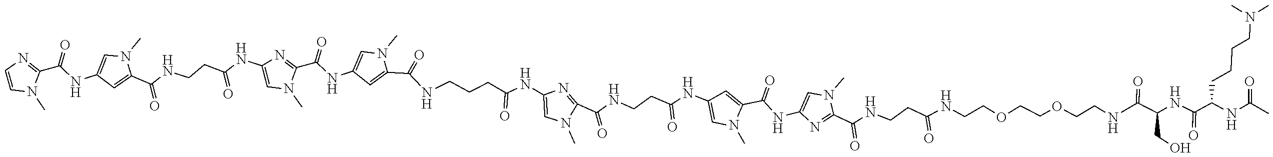 [SMILES: CC(=O)N[C@@H](CCCCN(C)C)C(=O)N[C@@H](CO)C(=O)NCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 80 |
| 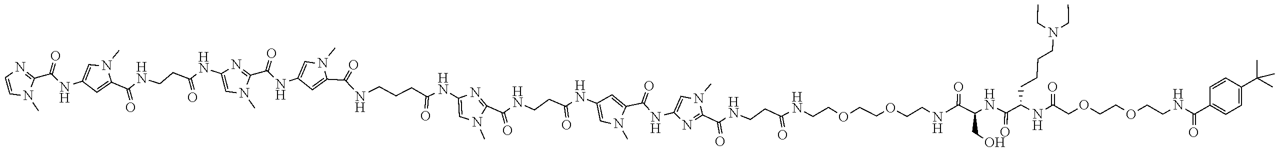 [SMILES: CCN(CC)CCCC[C@H](NC(=O)COCCOCCNC(=O)c1ccc(C(C)(C)C)cc1)C(=O)N[C@@H](CO)C(=O)NCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 81 |
| 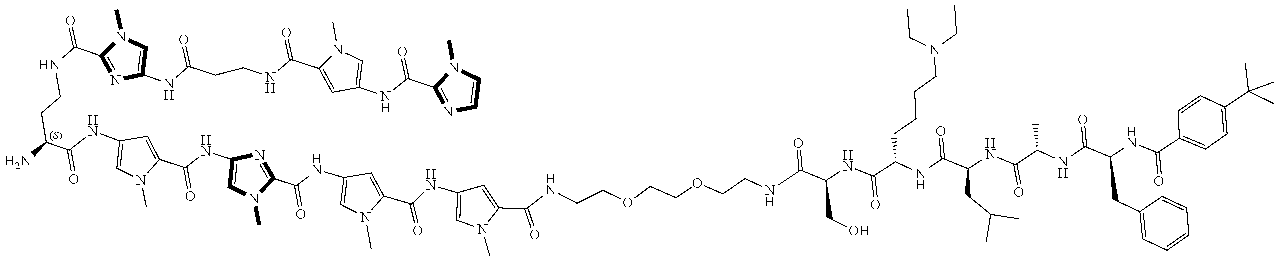 | 82 |
| 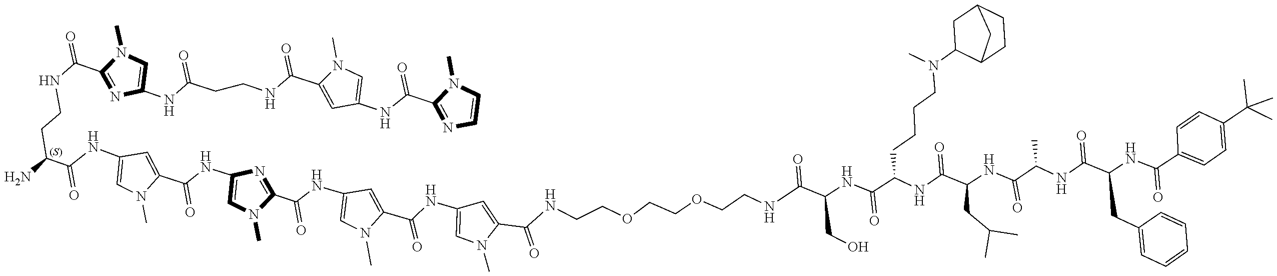 | 83 |
| 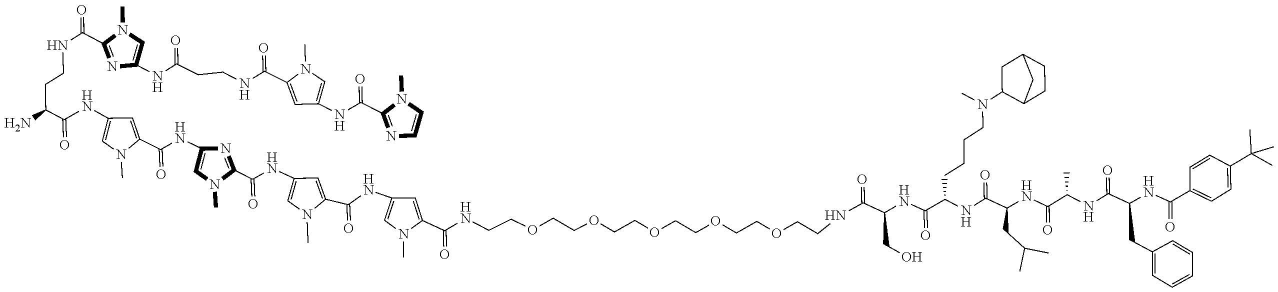 | 84 |
| 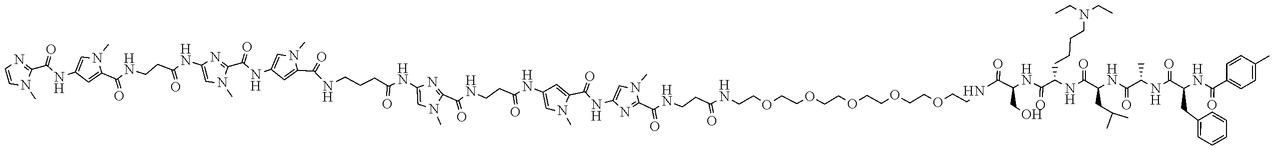 [SMILES: CCN(CC)CCCC[C@H](NC(=O)[C@H](CC(C)C)NC(=O)[C@H](C)NC(=O)[C@H](Cc1ccccc1)NC(=O)c1ccc(C)cc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 85 |
| 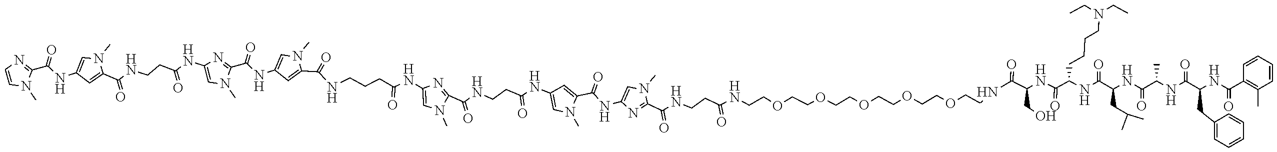 [SMILES: CCN(CC)CCCC[C@H](NC(=O)[C@H](CC(C)C)NC(=O)[C@H](C)NC(=O)[C@H](Cc1ccccc1)NC(=O)c1ccccc1C)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 86 |
| 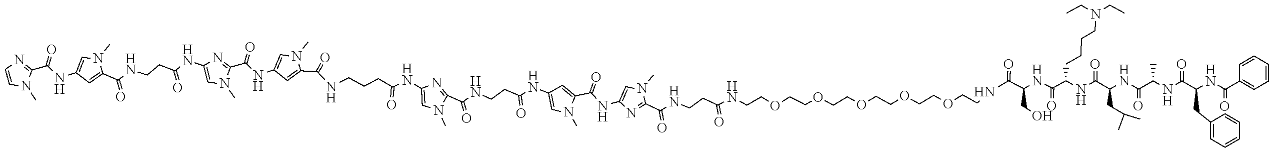 [SMILES: CCN(CC)CCCC[C@H](NC(=O)[C@H](CC(C)C)NC(=O)[C@H](C)NC(=O)[C@H](Cc1ccccc1)NC(=O)c1ccccc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 87 |
| 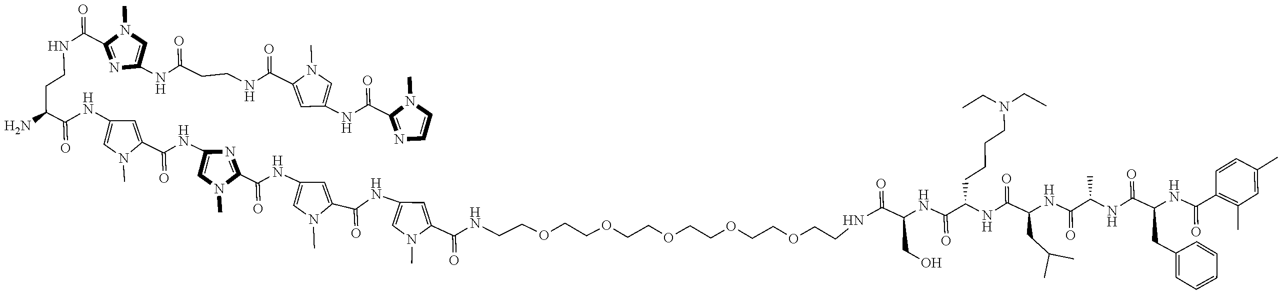 | 88 |
| 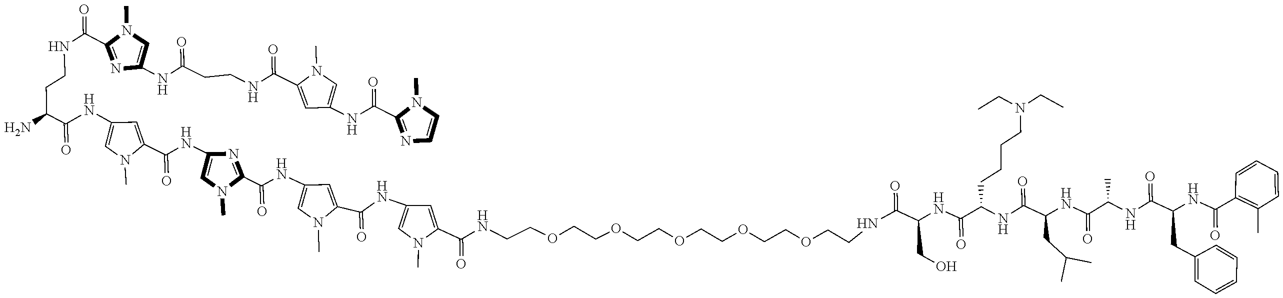 | 89 |
| 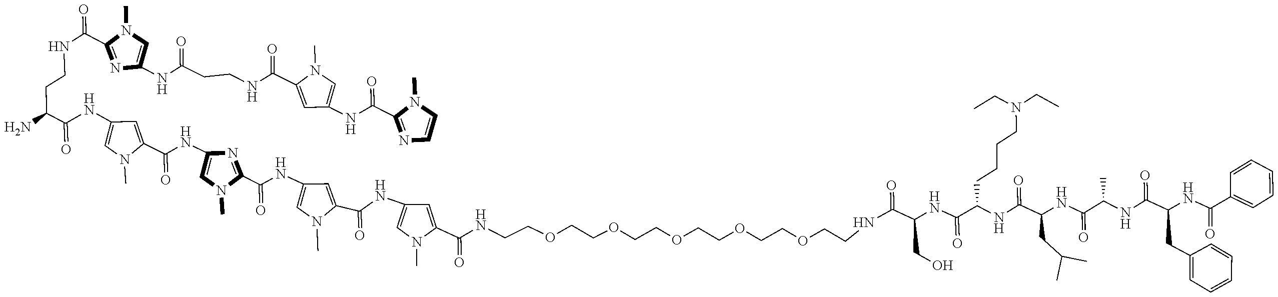 | 90 |
| 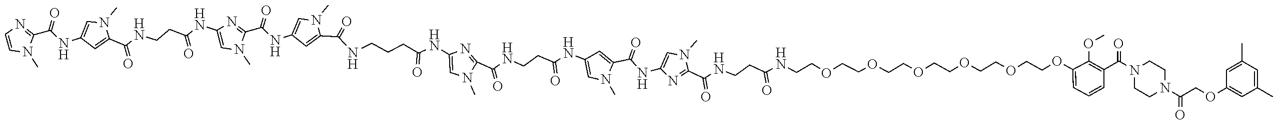 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cc(C)cc(C)c2)CC1] | 91 |
| 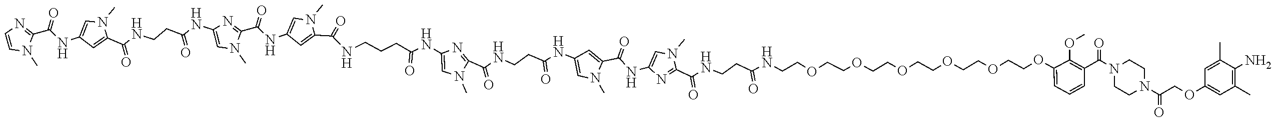 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cc(C)c(N)c(C)c2)CC1] | 92 |
| 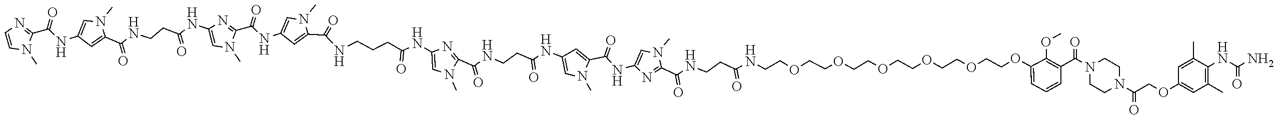 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cc(C)c(NC(N)=O)c(C)c2)CC1] | 93 |
| 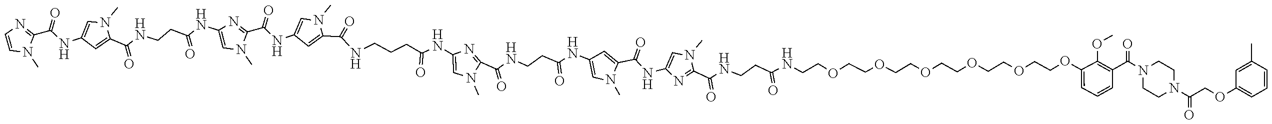 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cccc(C)c2)CC1] | 94 |
| 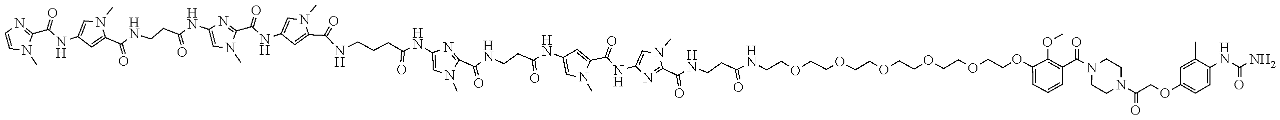 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2ccc(NC(N)=O)c(C)c2)CC1] | 95 |
| 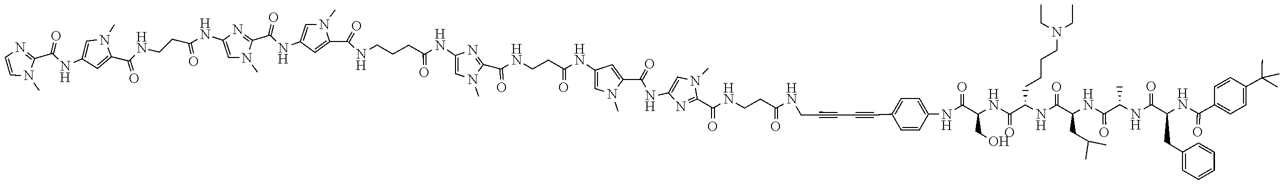 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)Nc1ccc(C#CC#CCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cc1] | 96 |
| 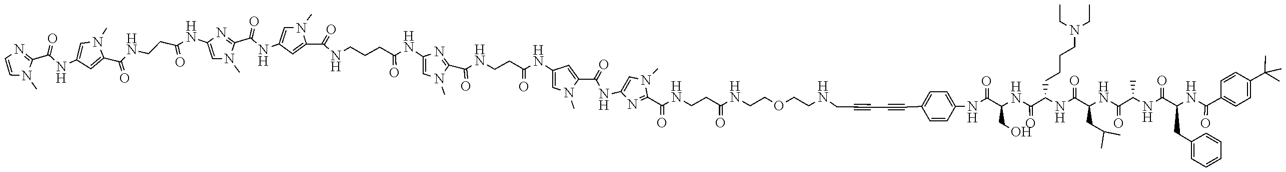 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)Nc1ccc(C#CC#CCNCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cc1] | 97 |
| 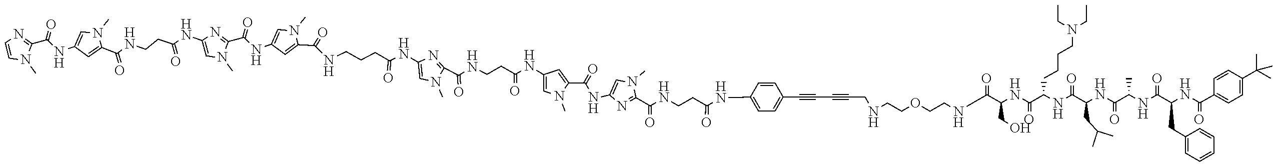 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCNCC#CC#Cc1ccc(NC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cc1] | 98 |
| 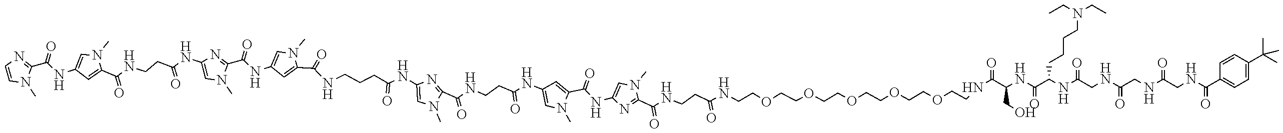 [SMILES: CCN(CC)CCCC[C@H](NC(=O)CNC(=O)CNC(=O)CNC(=O)c1ccc(C(C)(C)C)cc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 99 |
| 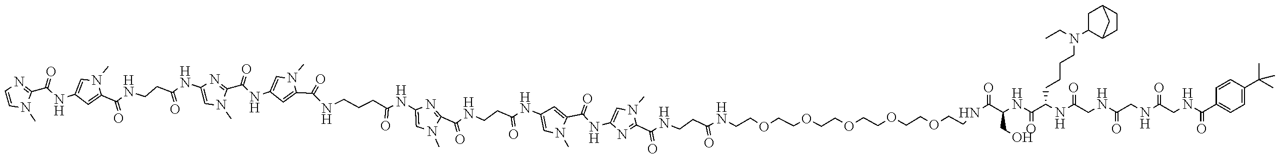 [SMILES: CCN(CCCC[C@H](NC(=O)CNC(=O)CNC(=O)CNC(=O)c1ccc(C(C)(C)C)cc1)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)C1CC2CCC1C2] | 100 |
| 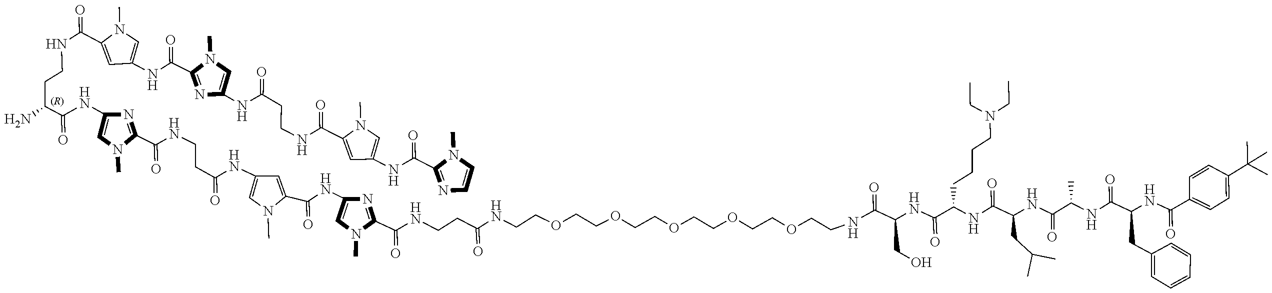 | 101 |
| 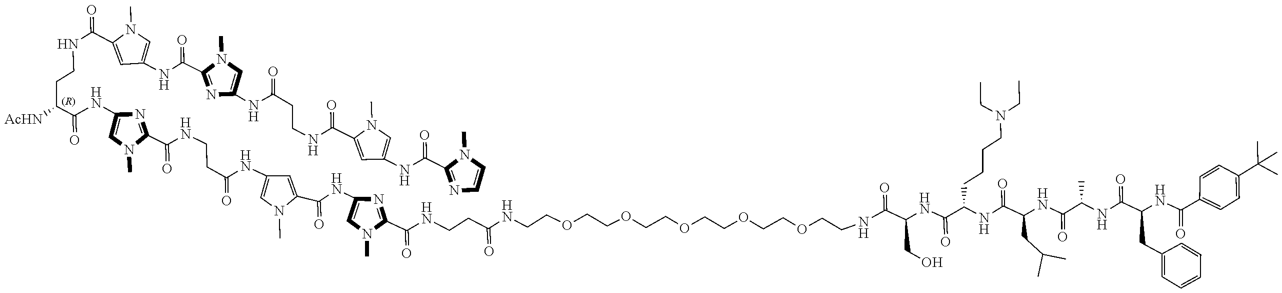 | 102 |
| 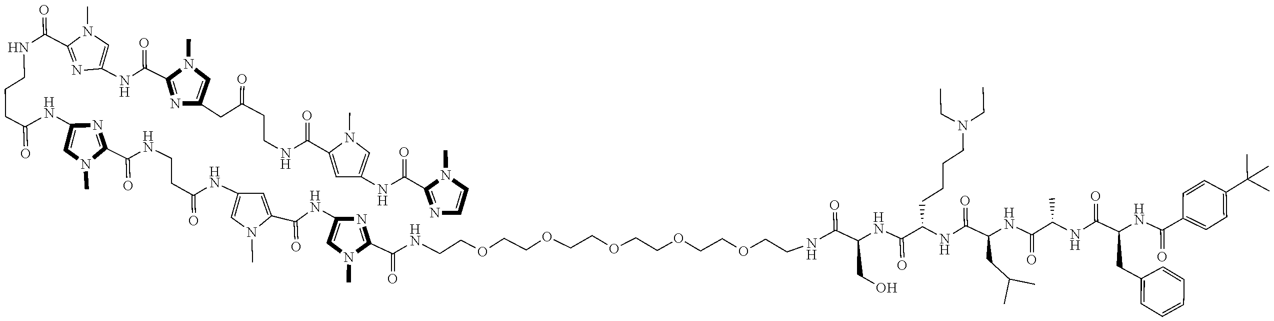 | 103 |
| 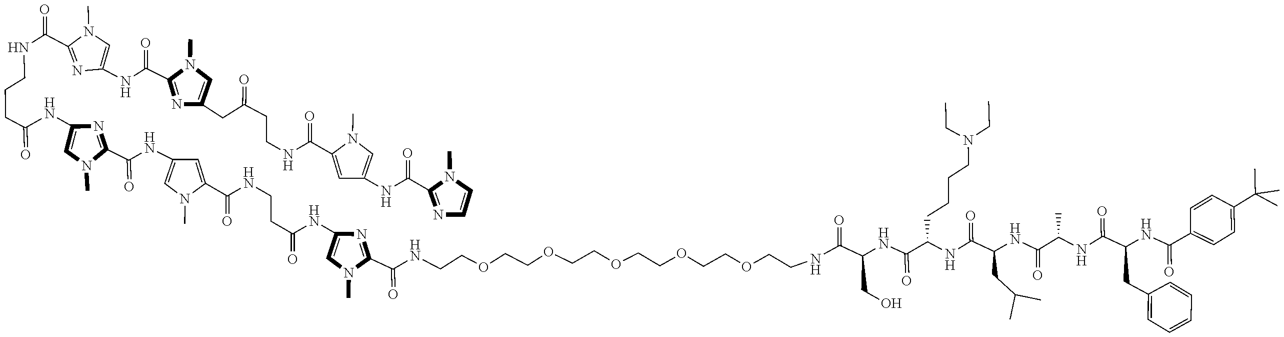 | 104 |
| 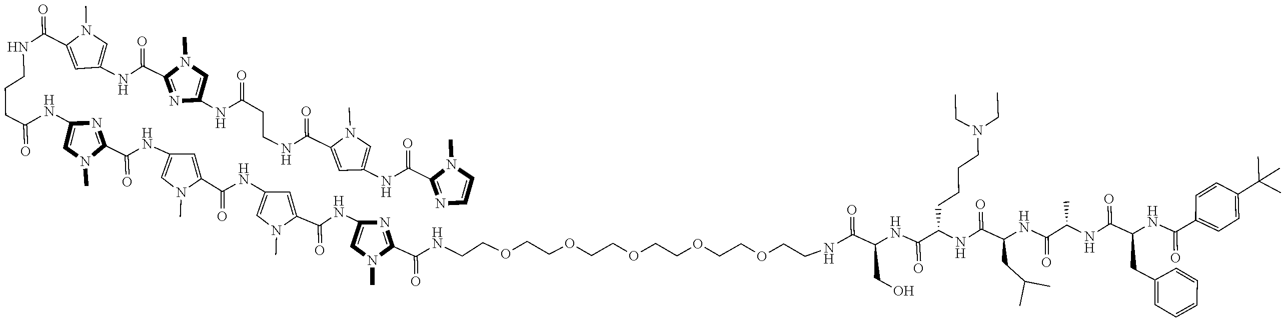 | 105 |
| 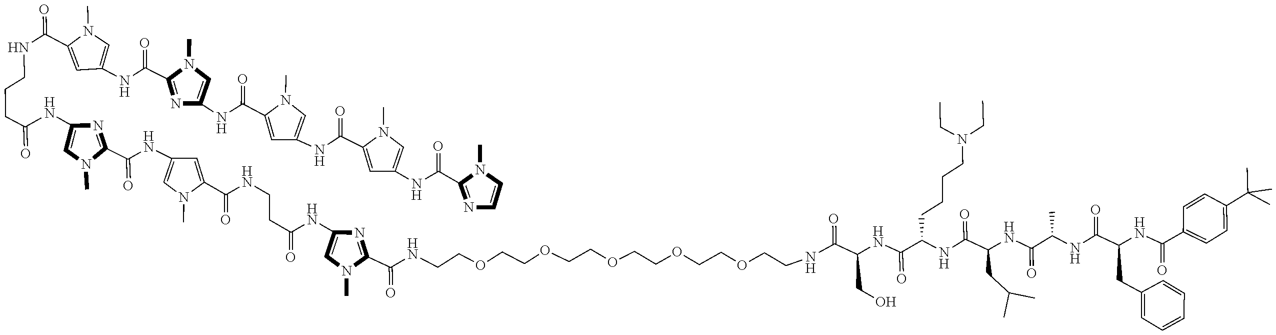 | 106 |
| 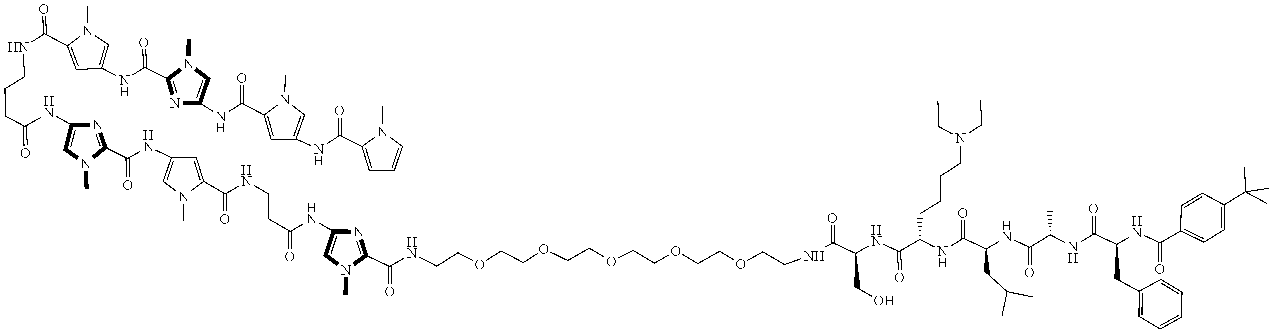 | 107 |
| 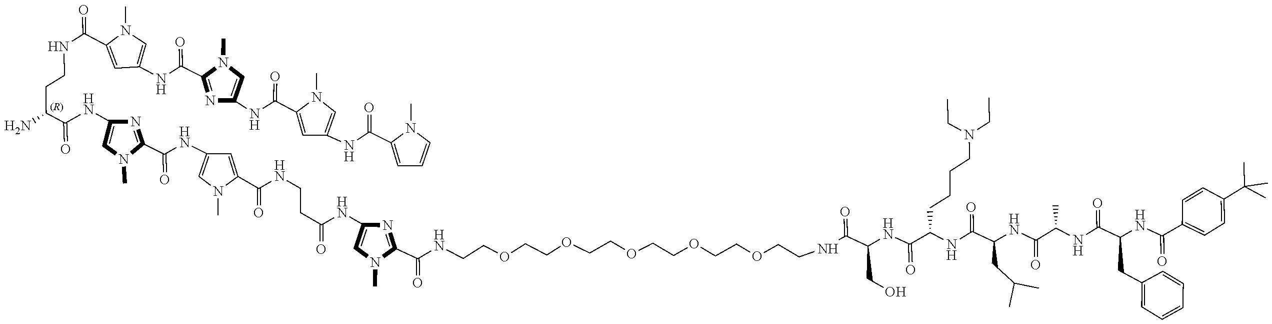 | 108 |
| 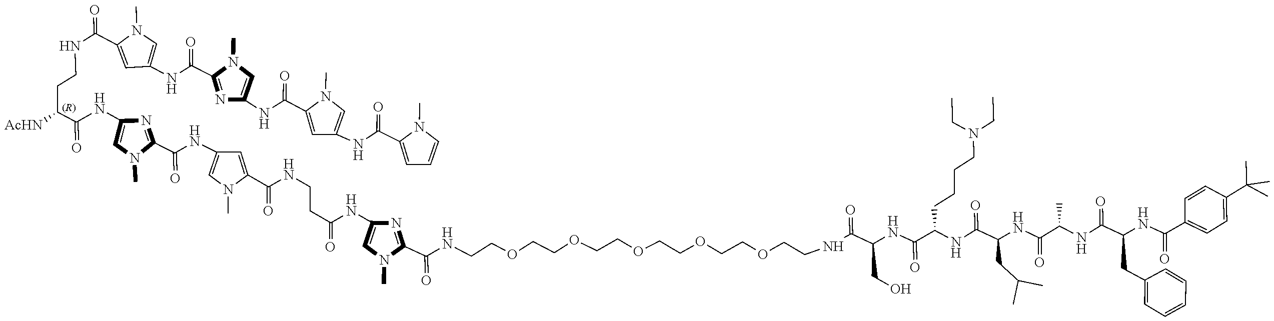 | 109 |
| 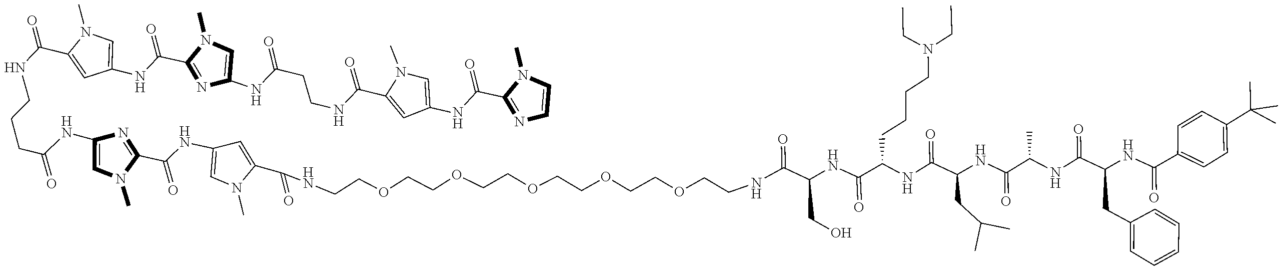 | 110 |
| 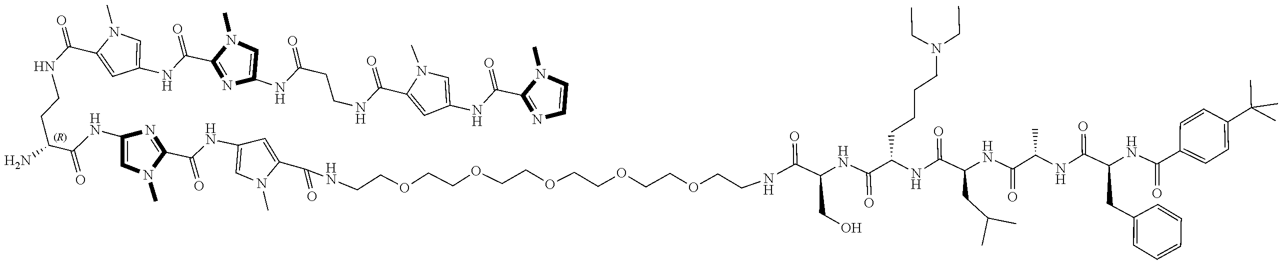 | 111 |
| 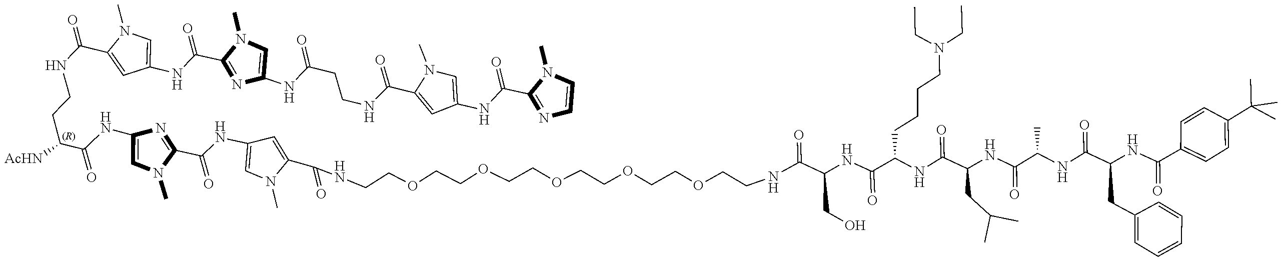 | 112 |
| 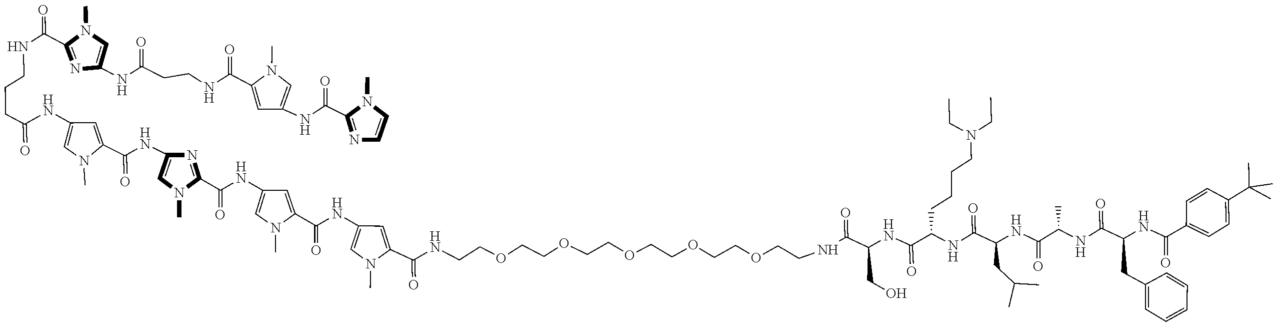 | 113 |
| 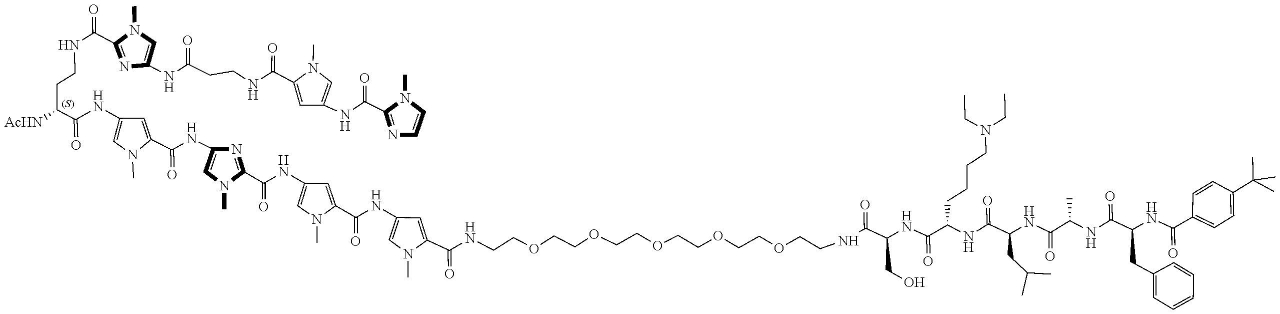 | 114 |
| 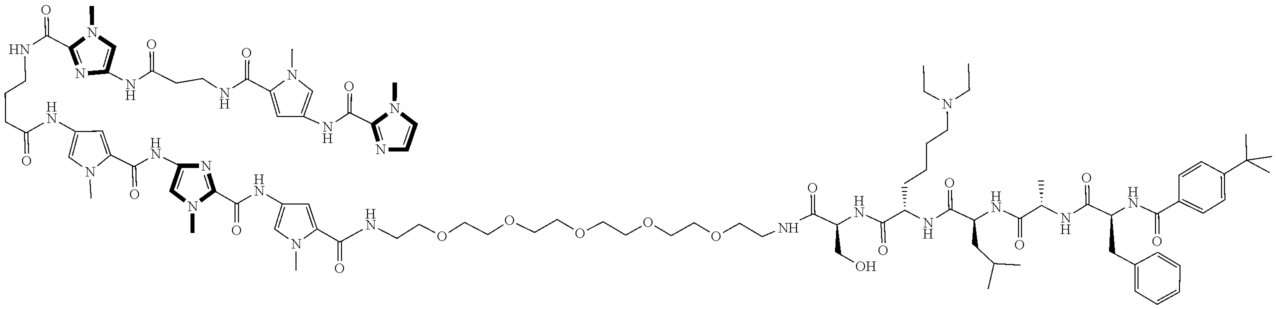 | 115 |
| 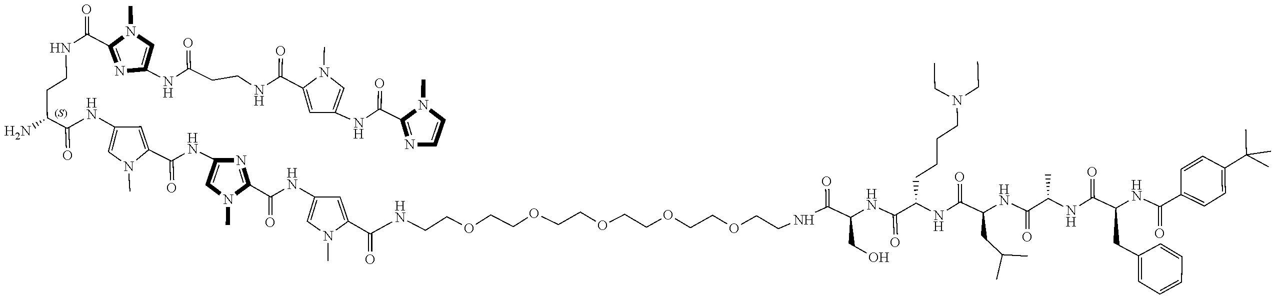 | 116 |
| 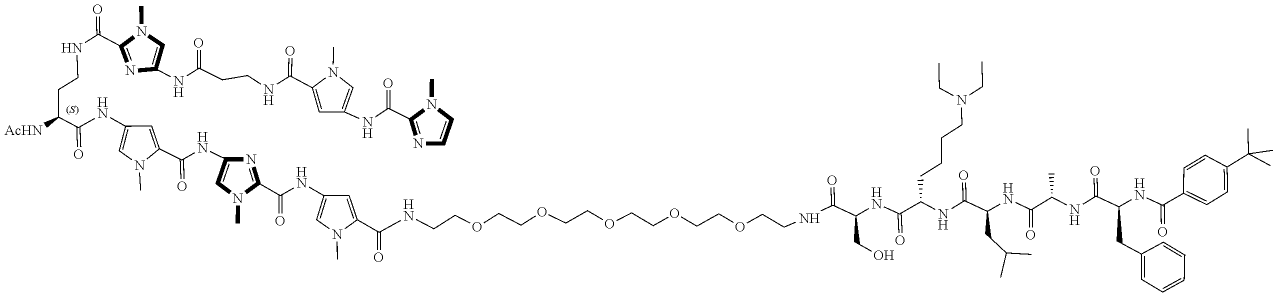 | 117 |
| 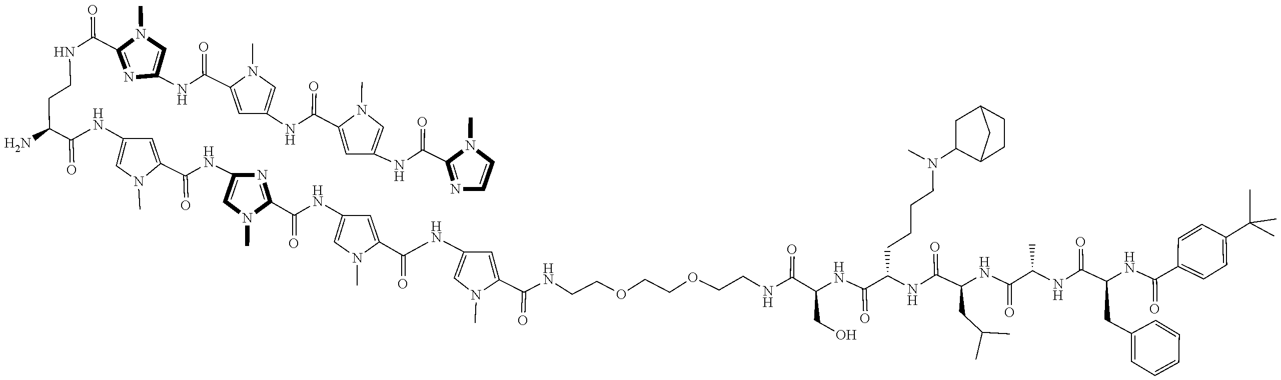 | 118 |
| 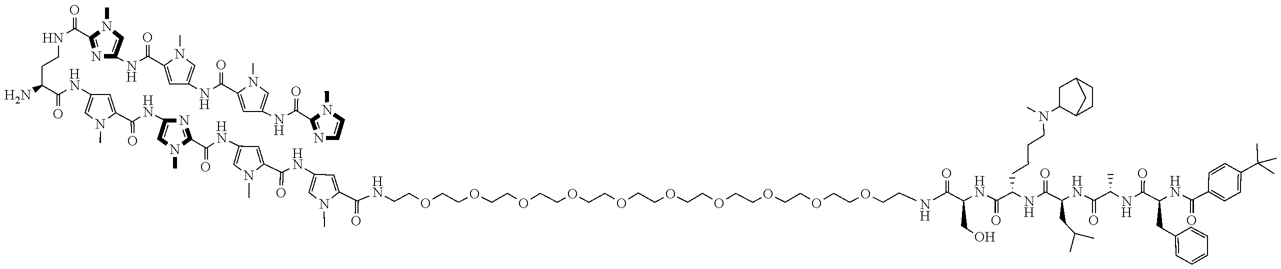 | 119 |
| 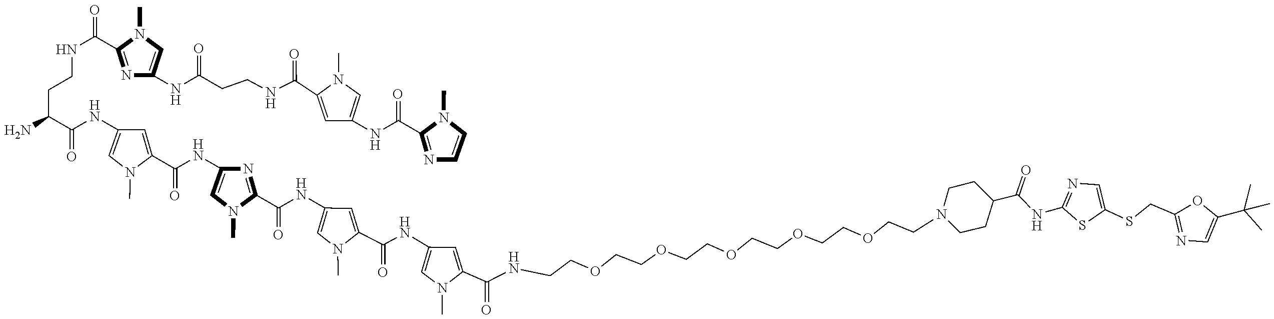 | 120 |
| 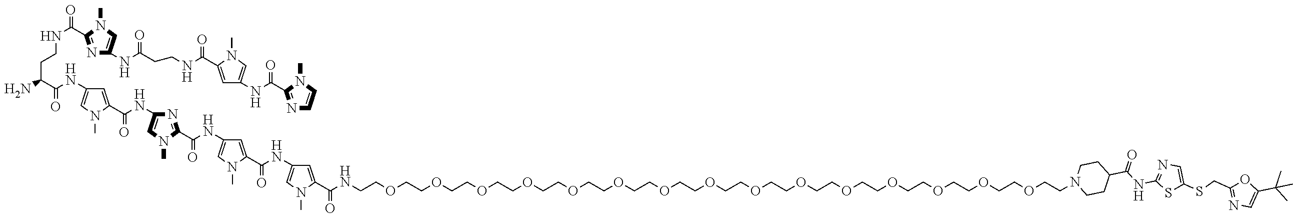 | 121 |
| 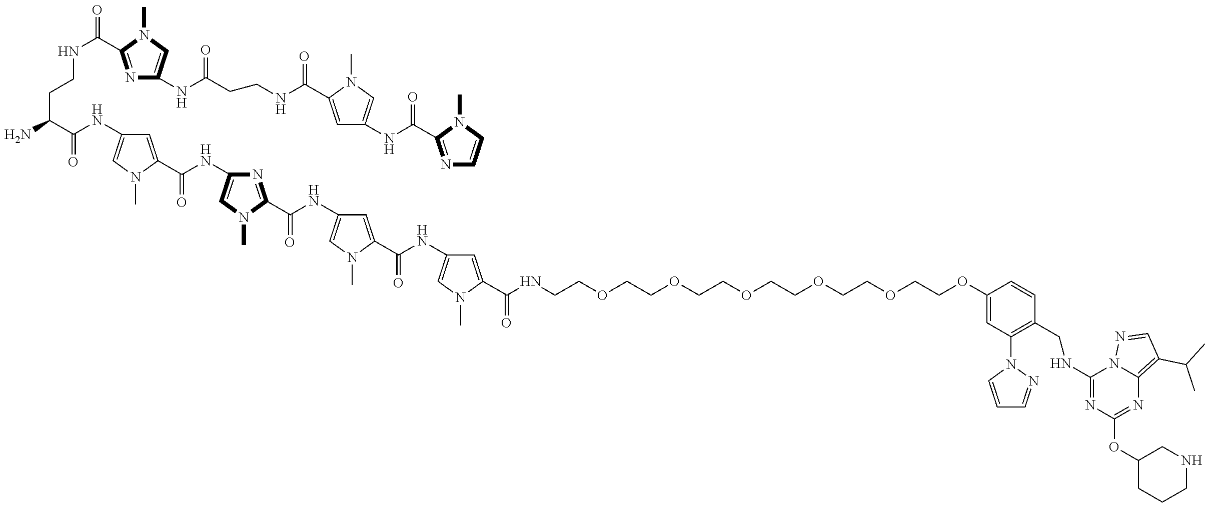 | 122 |
| 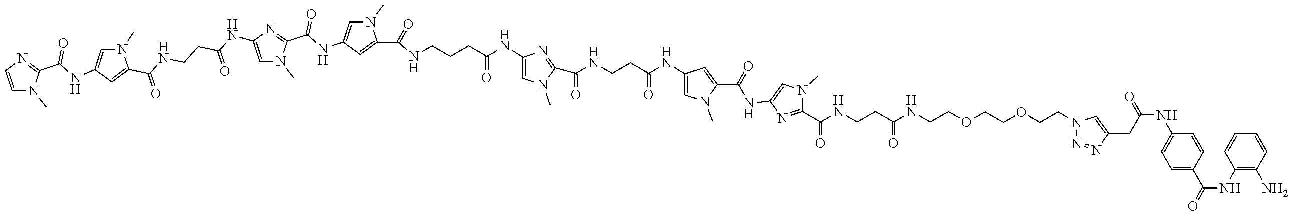 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCn4cc(CC(=O)Nc5ccc(C(=O)Nc6ccccc6N)cc5)nn4)n3)n(C)c2)n1] | 123 |
| 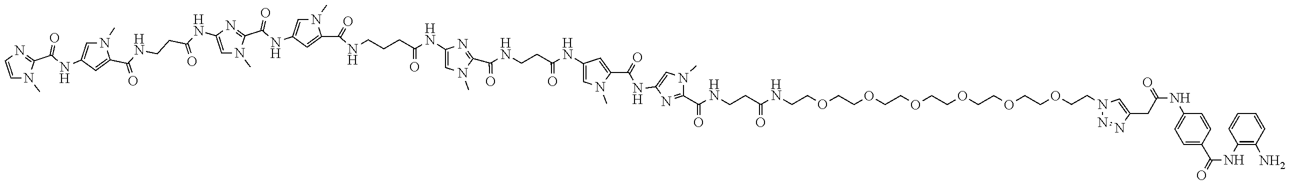 | 124 |
| 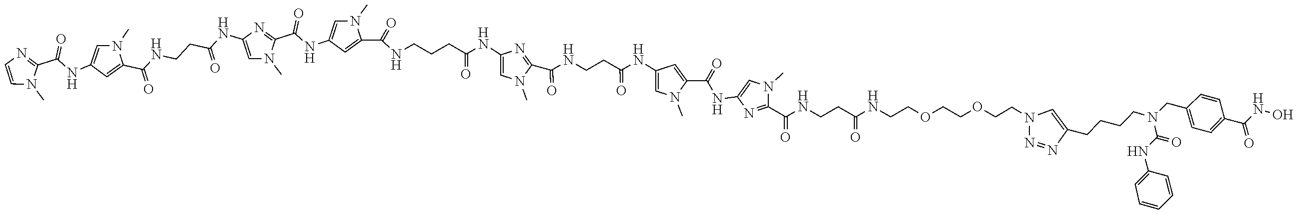 | 125 |
| 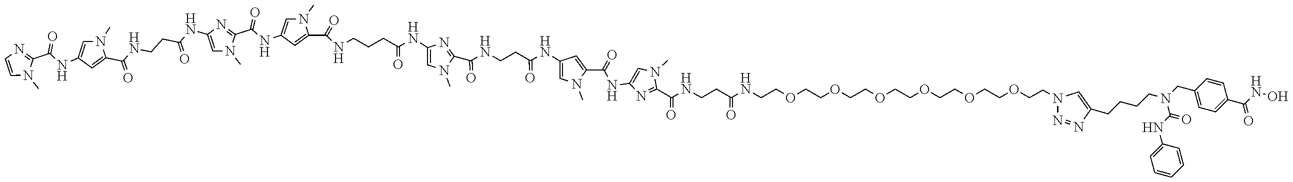 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOCCn4cc(CCCCN(Cc5ccc(C(=O)NO)cc5)C(=O)Nc5ccccc5)nn4)n3)n(C)c2)n1] | 126 |
| 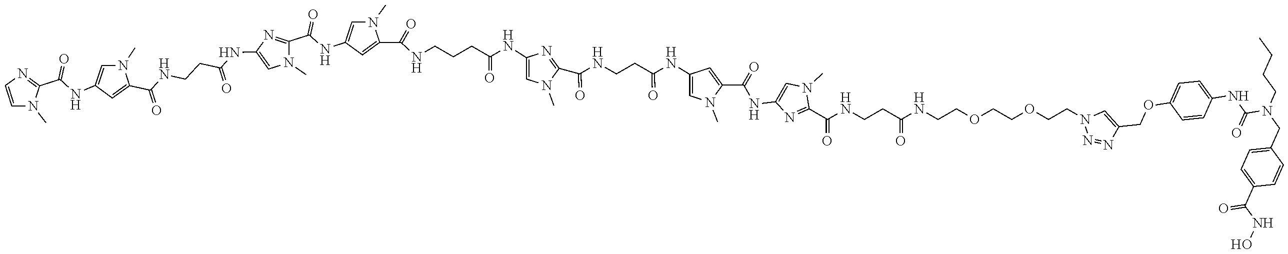 [SMILES: CCCCN(Cc1ccc(C(=O)NO)cc1)C(=O)Nc1ccc(OCc2cn(CCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)nn2)cc1] | 127 |
| 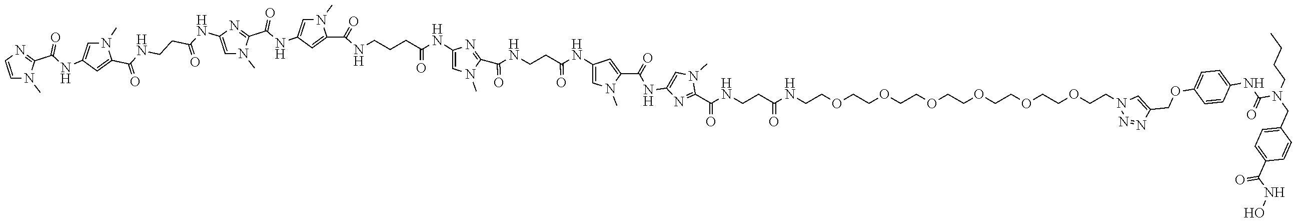 [SMILES: CCCCN(Cc1ccc(C(=O)NO)cc1)C(=O)Nc1ccc(OCc2cn(CCOCCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)nn2)cc1] | 128 |
| 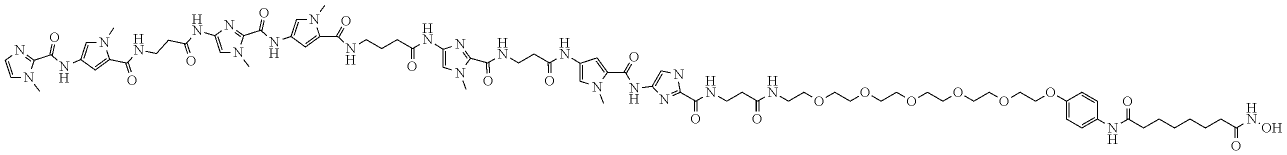 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3c[nH]c(C(=O)NCCC(=O)NCCOCCOCCOCCOCCOCCOc4ccc(NC(=O)CCCCCCC(=O)NO)cc4)n3)n(C)c2)n1] | 129 |
| 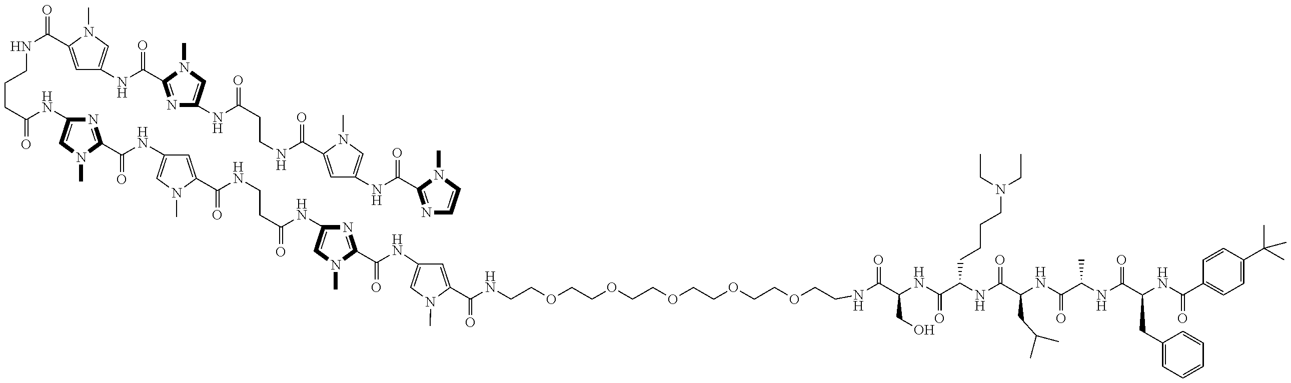 | 130 |
| 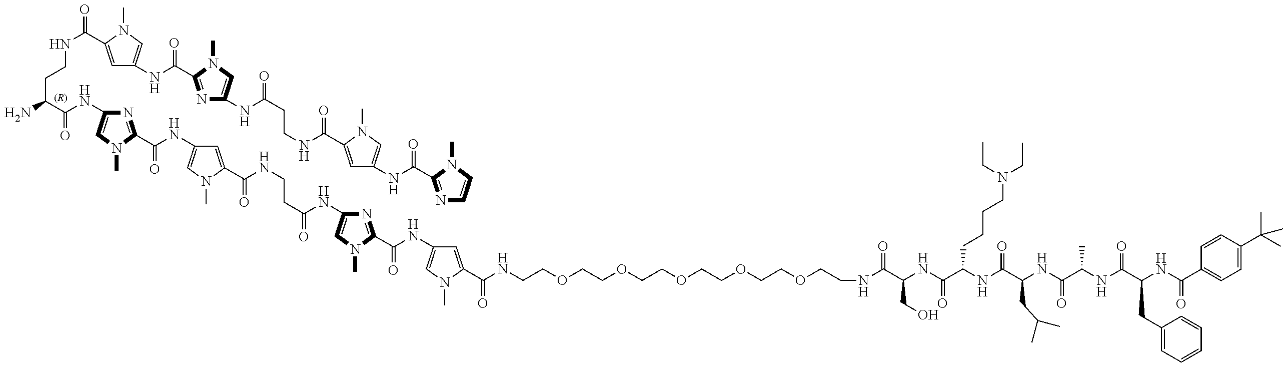 | 131 |
| 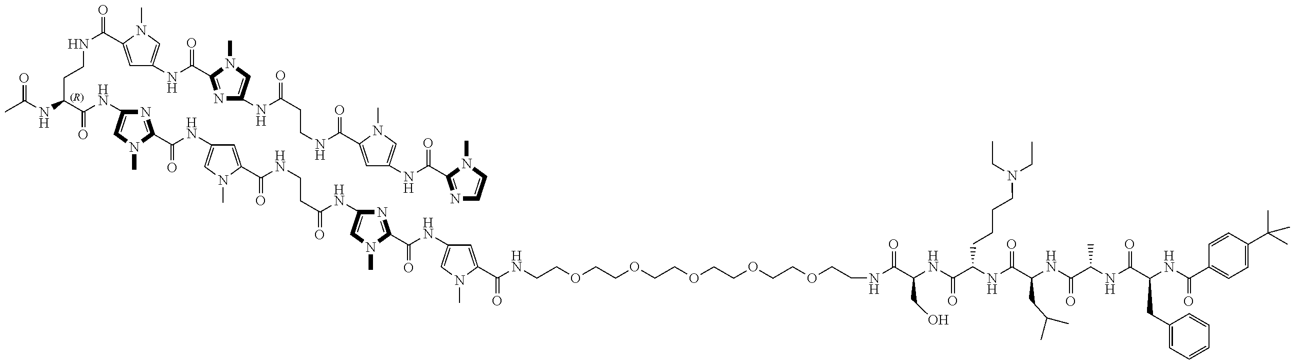 | 132 |
| 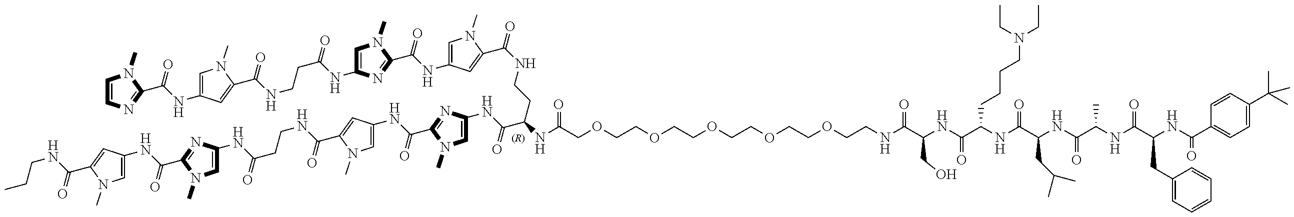 | 133 |
| 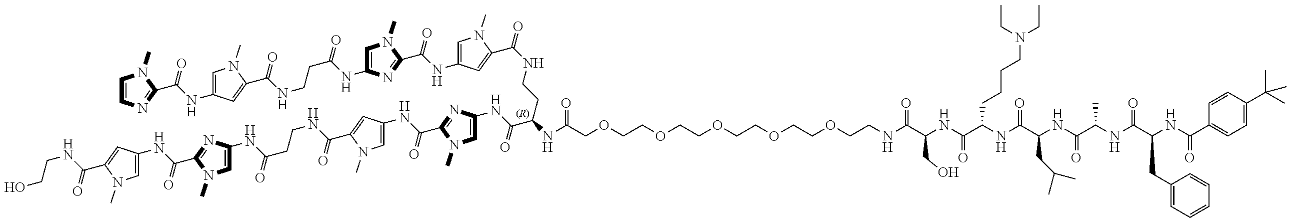 | 134 |
| 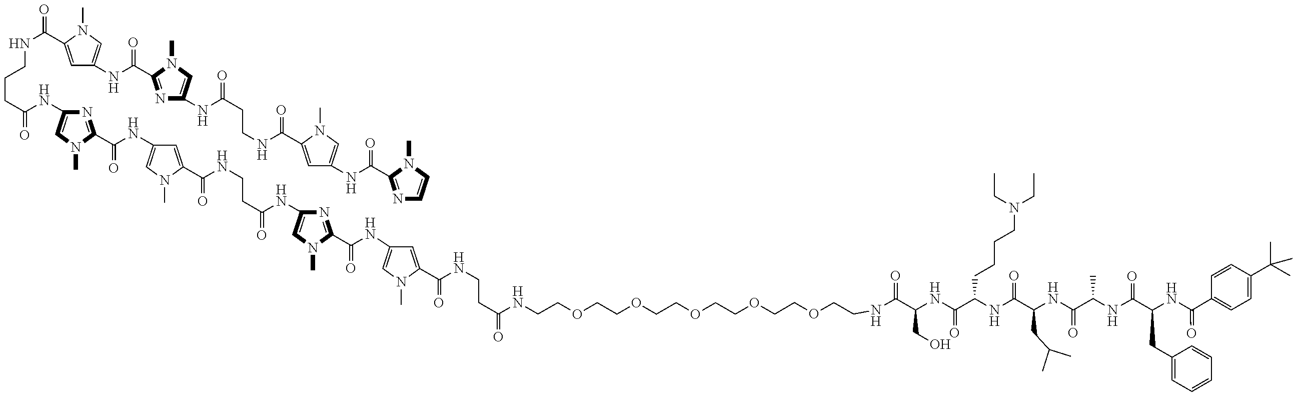 | 135 |
| 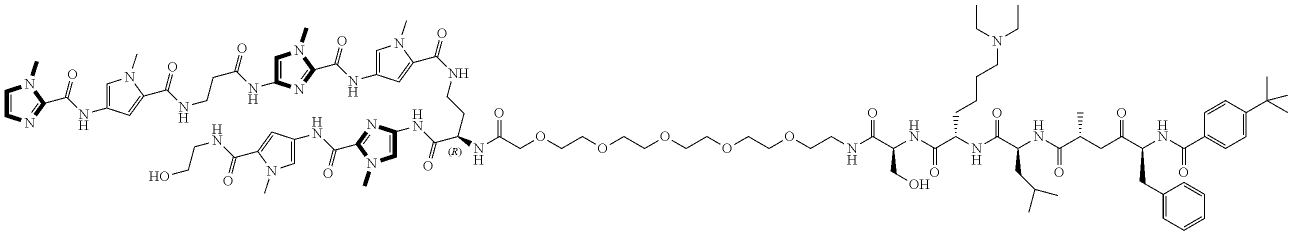 | 136 |
| 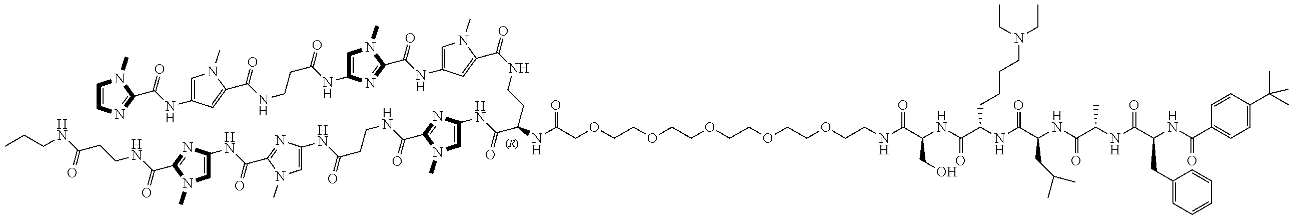 | 137 |
| 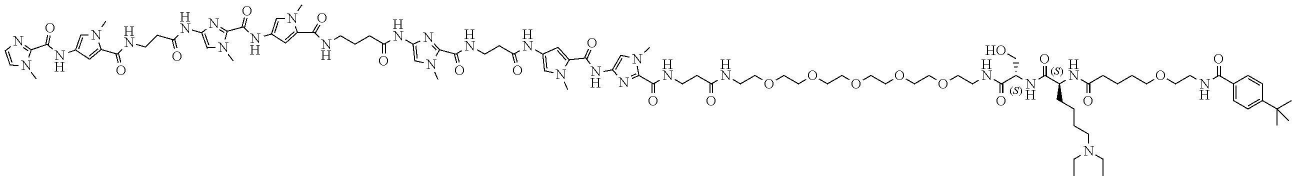 | 138 |
| 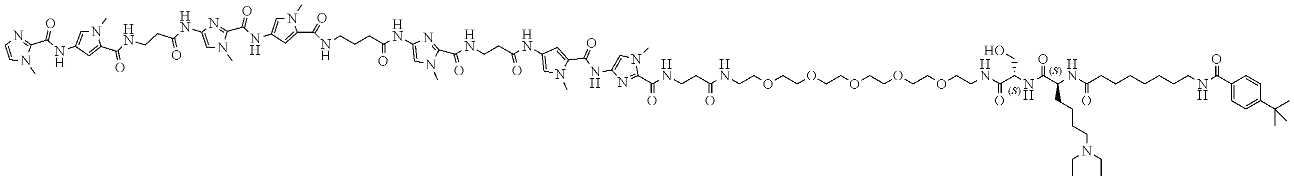 | 139 |
| 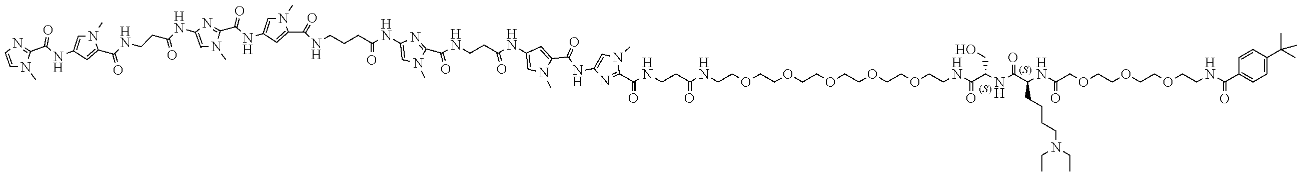 | 140 |
| 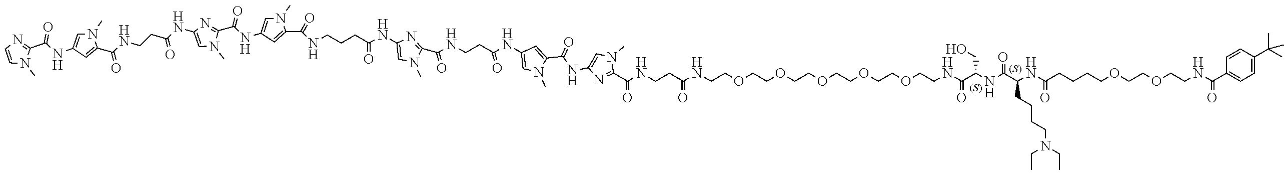 | 141 |
| 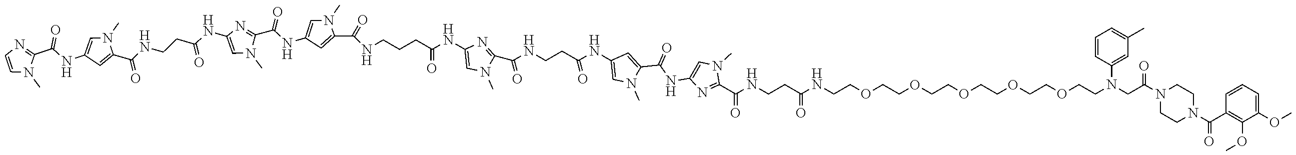 [SMILES: COc1cccc(C(=O)N2CCN(C(=O)CN(CCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)c3cccc(C)c3)CC2)c1OC] | 142 |
| 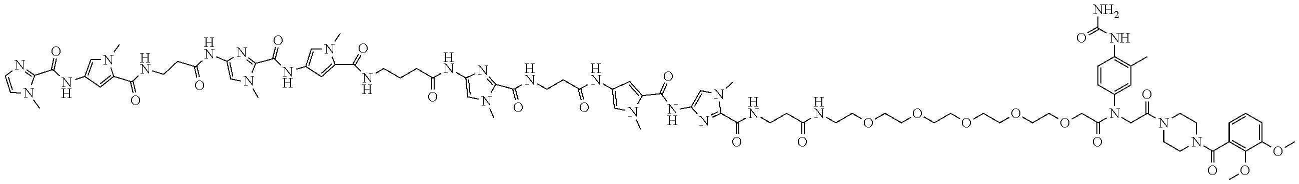 [SMILES: COc1cccc(C(=O)N2CCN(C(=O)CN(C(=O)COCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)c3ccc(NC(N)=O)c(C)c3)CC2)c1OC] | 143 |
| 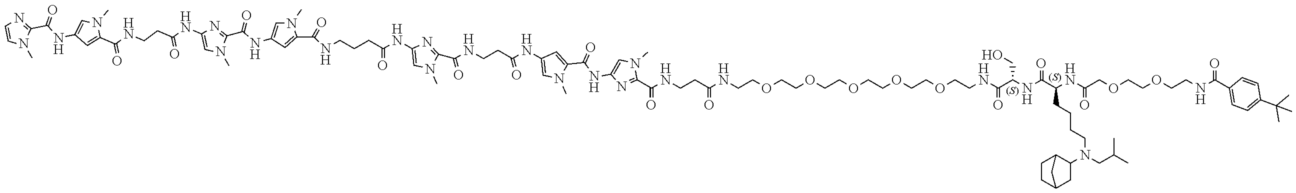 | 144 |
| 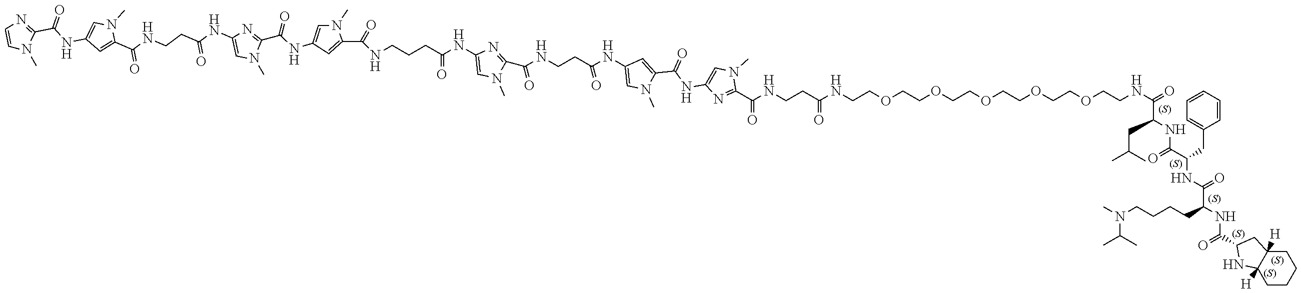 | 145 |
| 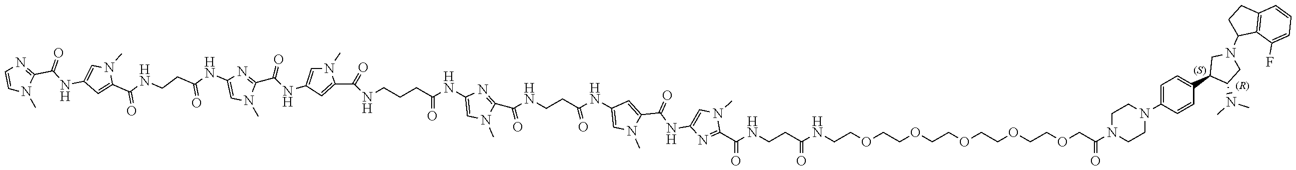 | 146 |
| 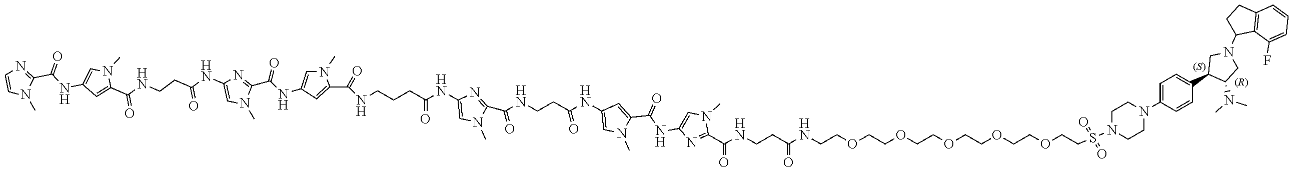 | 147 |
| 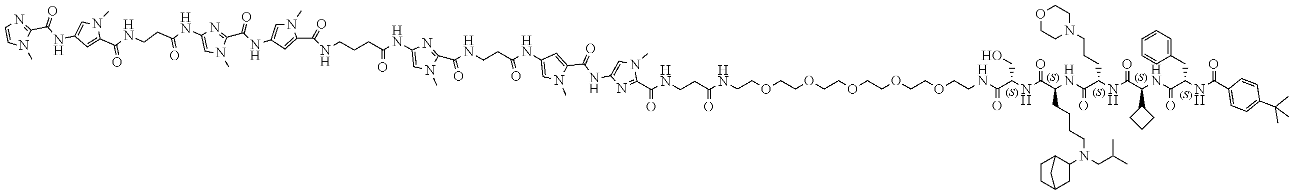 | 148 |
| 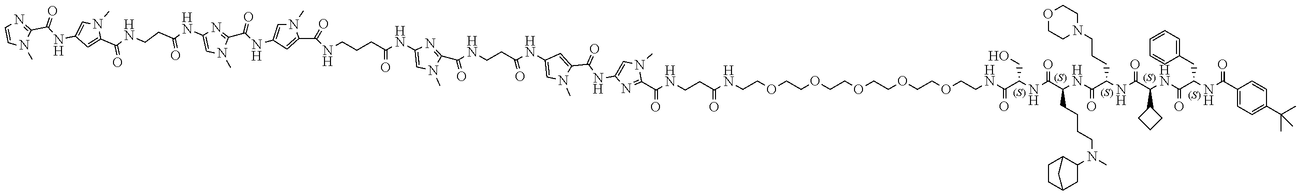 | 149 |
| 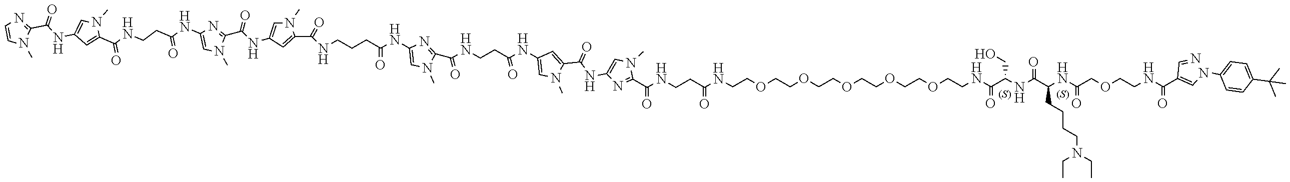 | 150 |
| 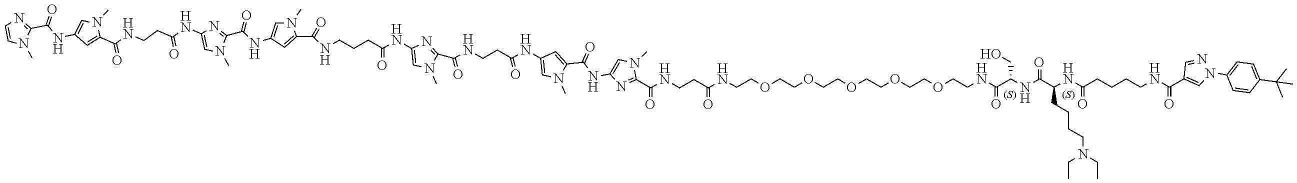 | 151 |
| 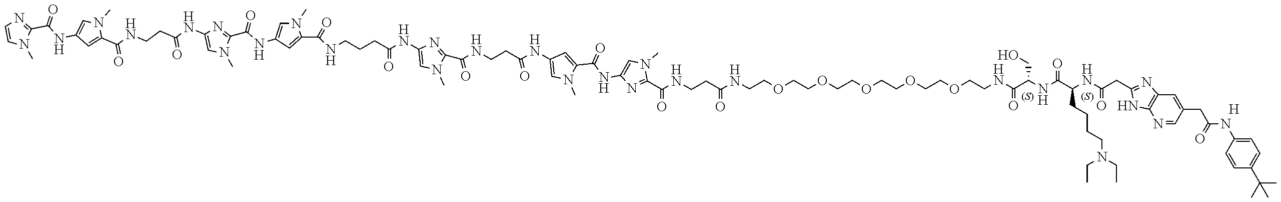 | 152 |
| 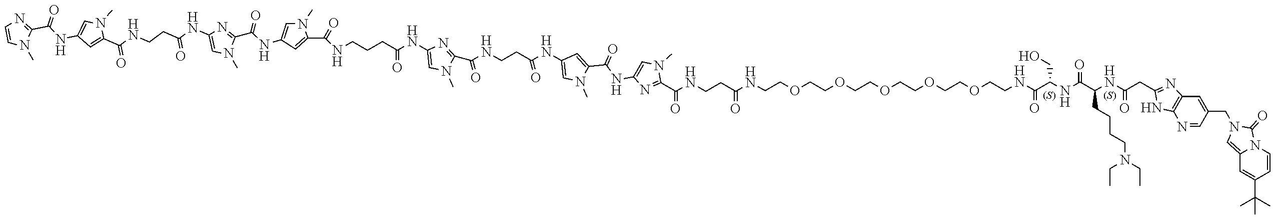 | 153 |
| 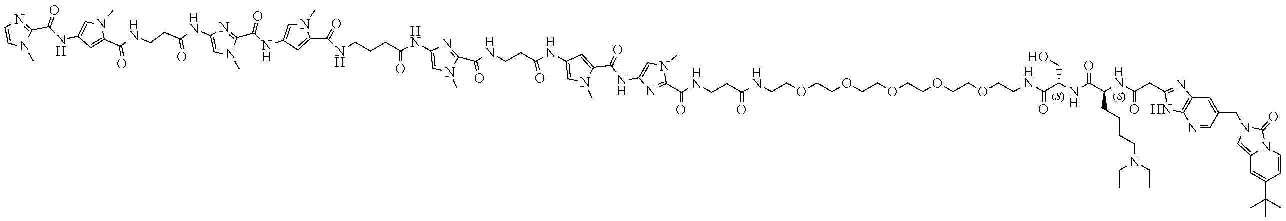 | 154 |
| 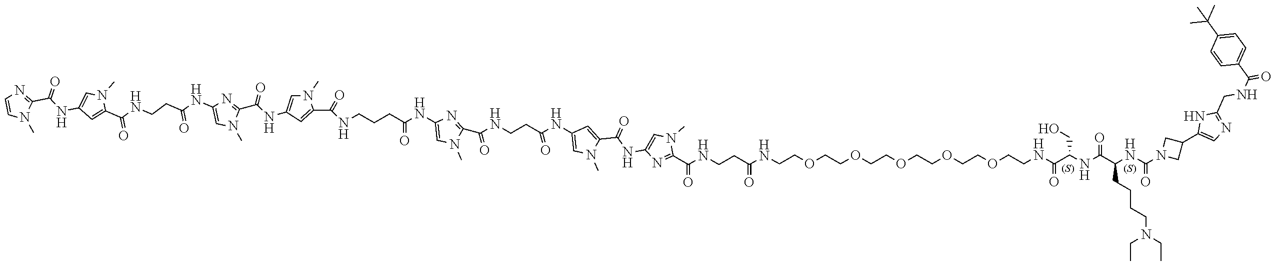 | 155 |
| 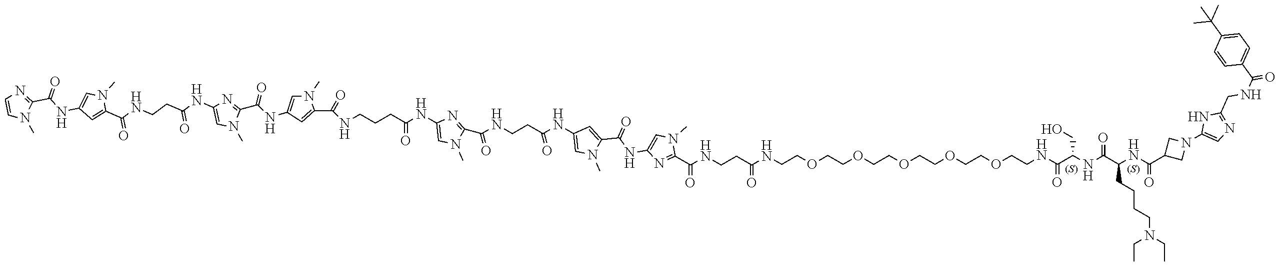 | 156 |
| 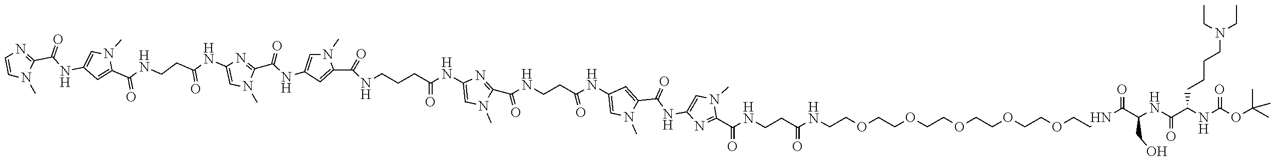 [SMILES: CCN(CC)CCCC[C@H](NC(=O)OC(C)(C)C)C(=O)N[C@@H](CO)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 157 |
| 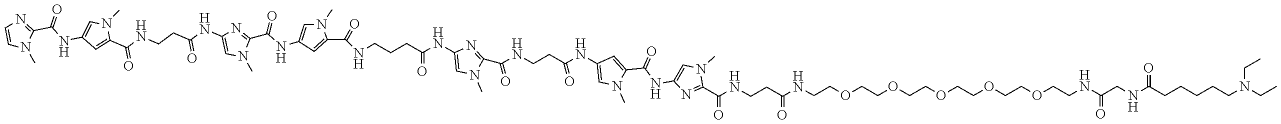 [SMILES: CCN(CC)CCCCCC(=O)NCC(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 158 |
| 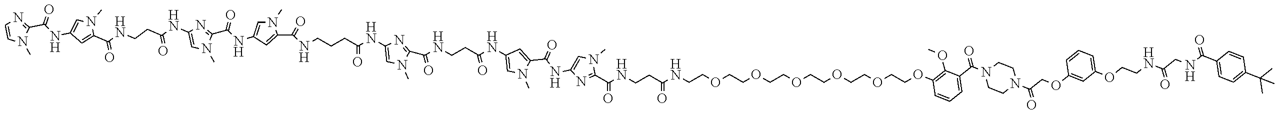 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cccc(OCCNC(=O)CNC(=O)c3ccc(C(C)(C)C)cc3)c2)CC1] | 159 |
| 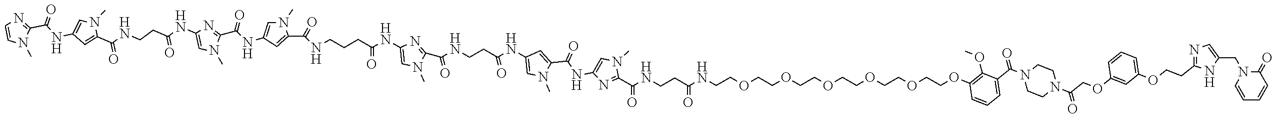 [SMILES: COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cccc(OCCc3ncc(Cn4ccccc4=O)[nH]3)c2)CC1] | 160 |
| 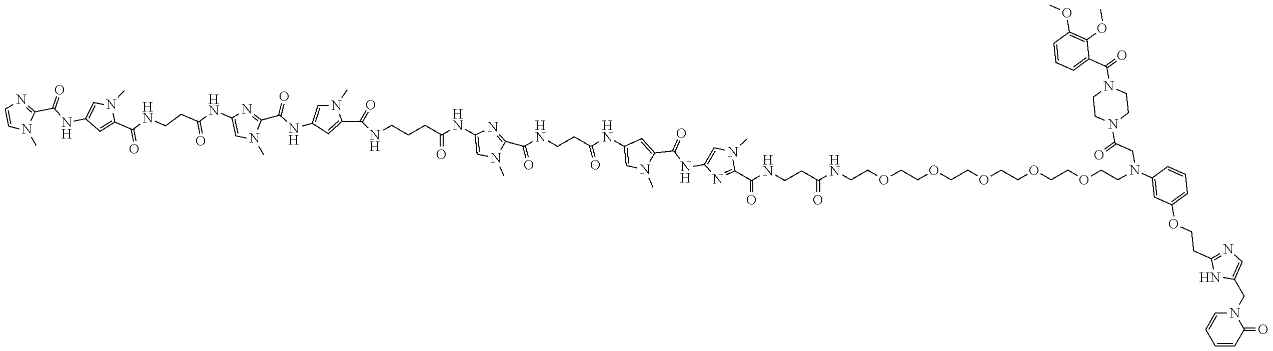 | 161 |
| 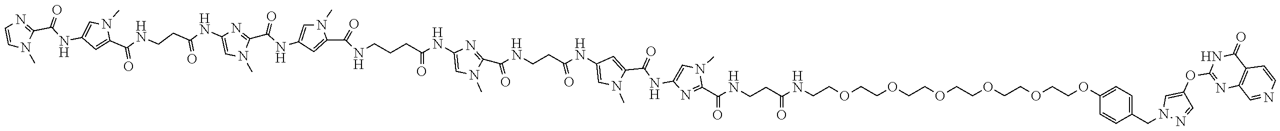 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCOc4ccc(Cn5cc(Oc6nc7cnccc7c(=O)[nH]6)cn5)cc4)n3)n(C)c2)n1] | 162 |
| 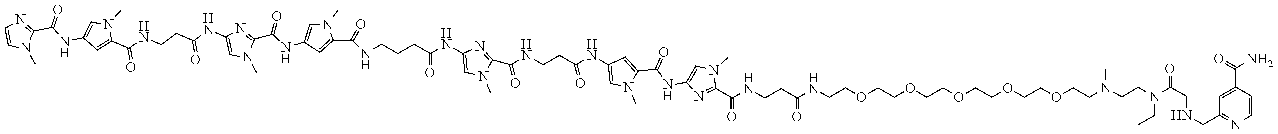 [SMILES: CCN(CCN(C)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)C(=O)CNCc1cc(C(N)=O)ccn1] | 163 |
| 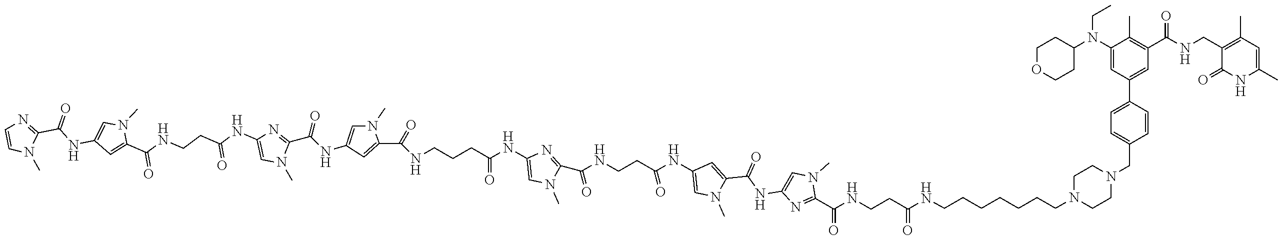 [SMILES: CCN(c1cc(-c2ccc(CN3CCN(CCCCCCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)cc2)cc(C(=O)NCc2c(C)cc(C)[nH]c2=O)c1C)C1CCOCC1] | 164 |
| 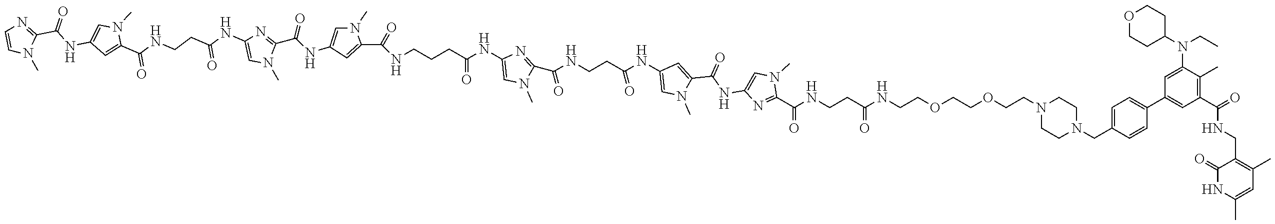 [SMILES: CCN(c1cc(-c2ccc(CN3CCN(CCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)cc2)cc(C(=O)NCc2c(C)cc(C)[nH]c2=O)c1C)C1CCOCC1] | 165 |
| 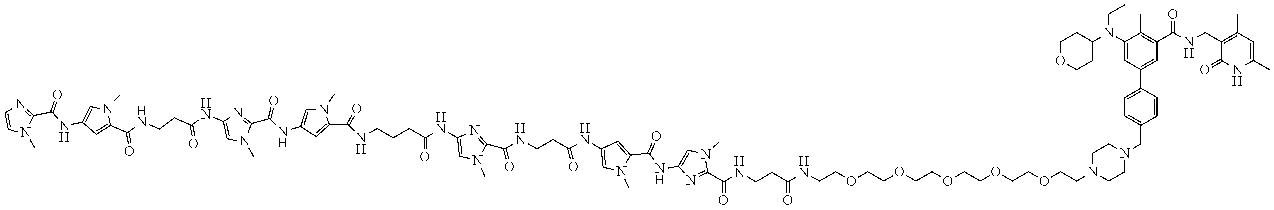 [SMILES: CCN(c1cc(-c2ccc(CN3CCN(CCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)cc2)cc(C(=O)NCc2c(C)cc(C)[nH]c2=O)c1C)C1CCOCC1] | 166 |
| 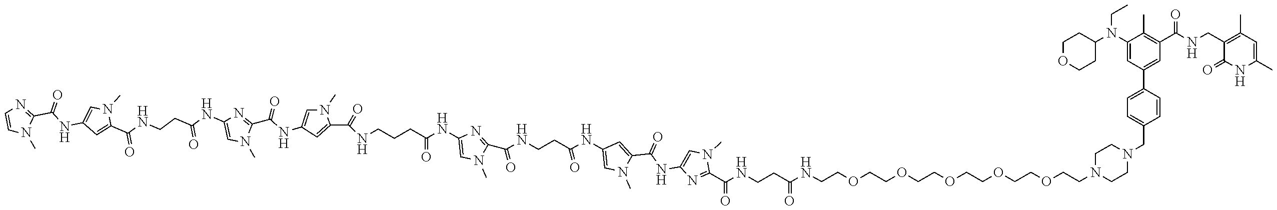 [SMILES: CCN(c1cc(-c2ccc(CN3CCN(CCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)cc2)cc(C(=O)NCc2c(C)cc(C)[nH]c2=O)c1C)C1CCOCC1] | 167 |
| 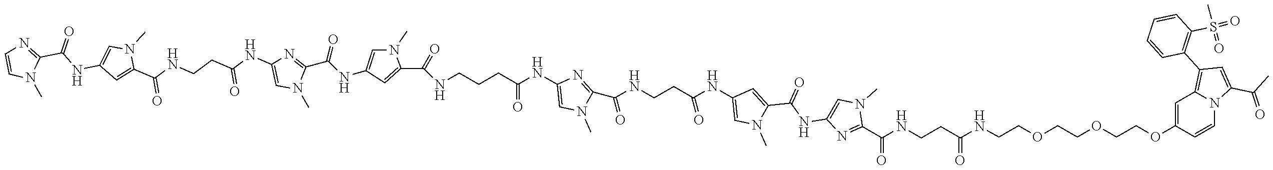 [SMILES: CC(=O)c1cc(-c2ccccc2S(C)(=O)=O)c2cc(OCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)ccn12] | 168 |
| 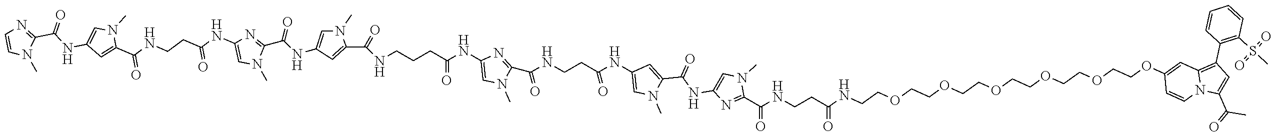 [SMILES: CC(=O)c1cc(-c2ccccc2S(C)(=O)=O)c2cc(OCCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)ccn12] | 169 |
| 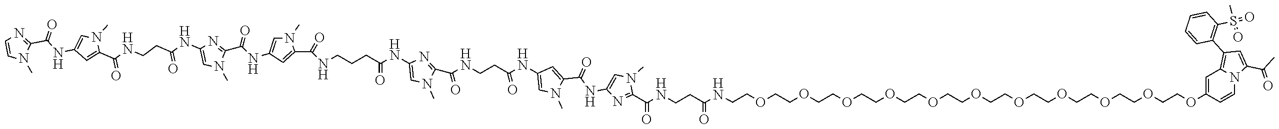 [SMILES: CC(=O)c1cc(-c2ccccc2S(C)(=O)=O)c2cc(OCCOCCOCCOCCOCCOCCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)ccn12] | 170 |
| 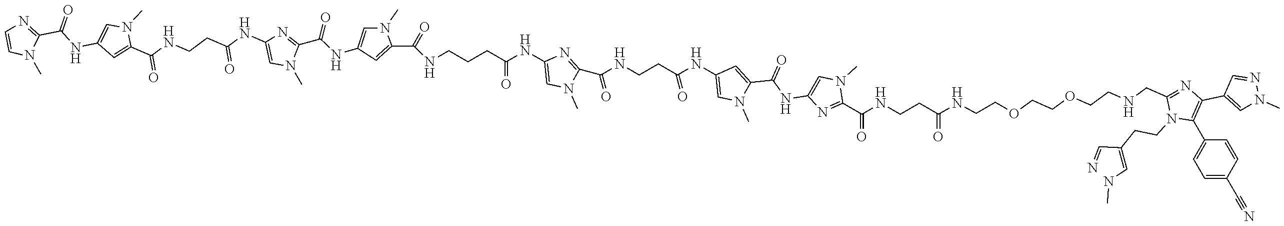 [SMILES: CN/C=C(\C=N)CCn1c(CNCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)nc(-c2cnn(C)c2)c1-c1ccc(C#N)cc1] | 171 |
| 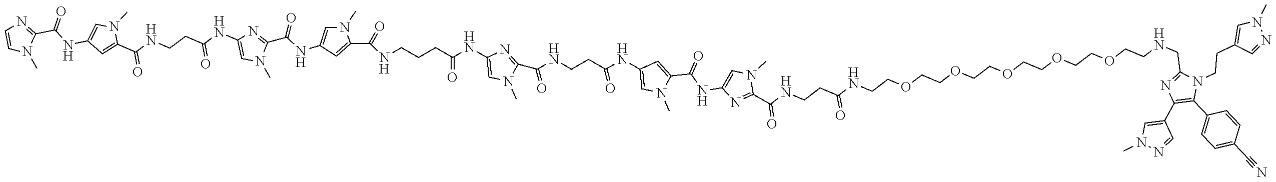 [SMILES: Cn1cc(CCn2c(CNCCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)nc(-c3cnn(C)c3)c2-c2ccc(C#N)cc2)cn1] | 172 |
| 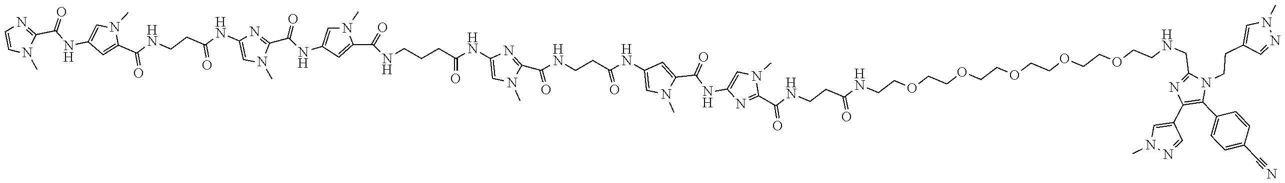 | 173 |
| 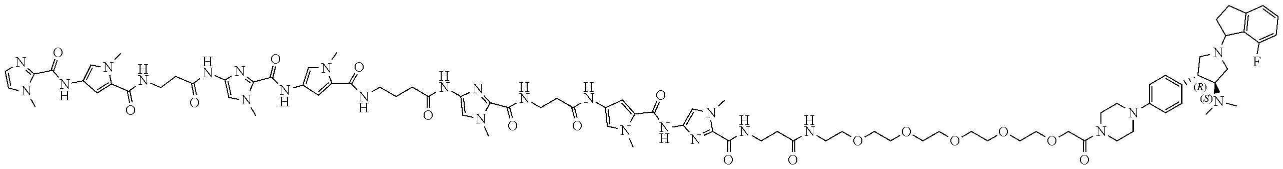 | 174 |
| 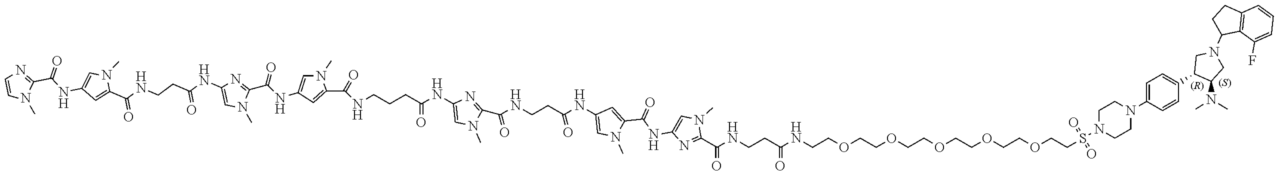 | 175 |
| 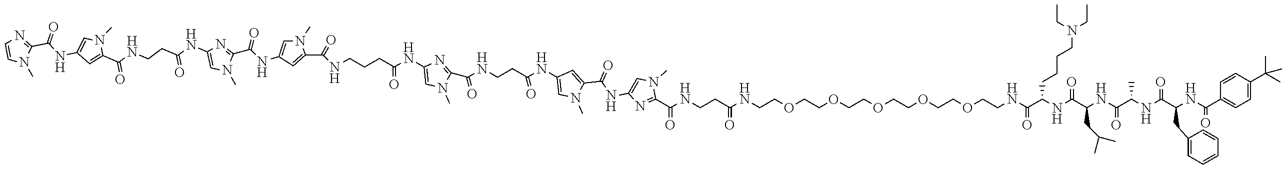 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)NCCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 176 |
| 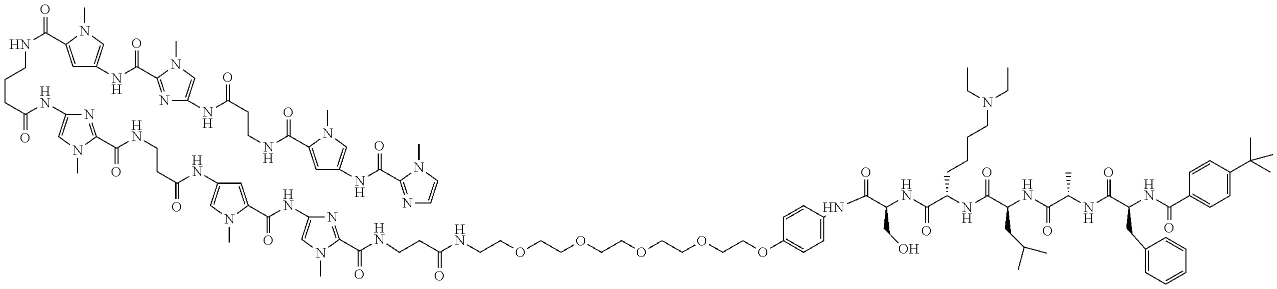 | 177 |
| 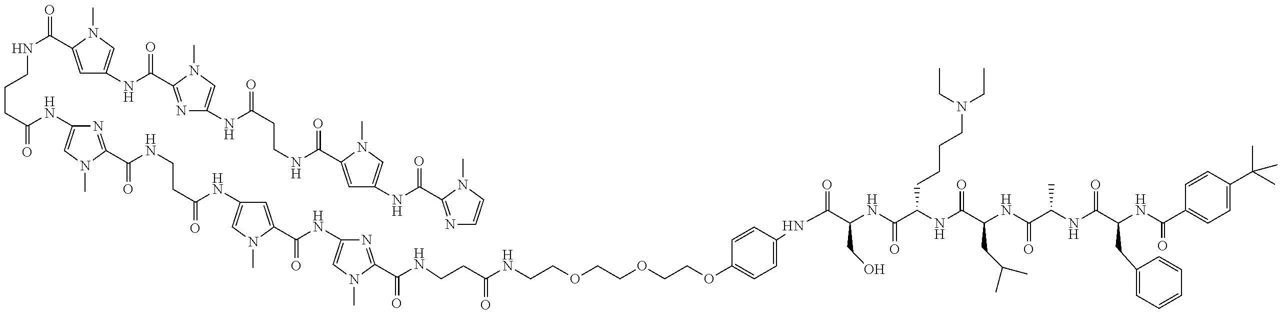 | 178 |
| 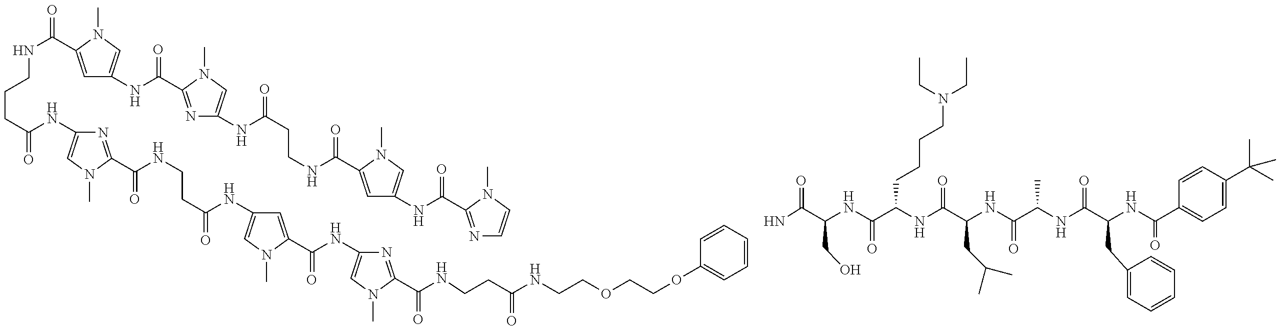 | 179 |
| 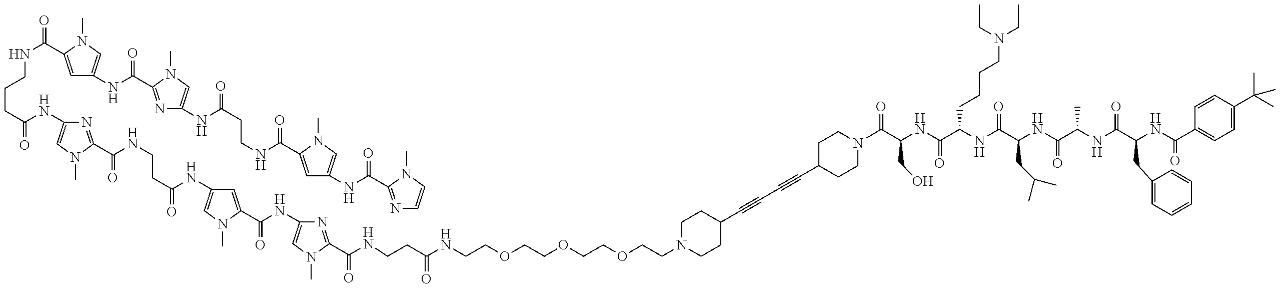 | 180 |
| 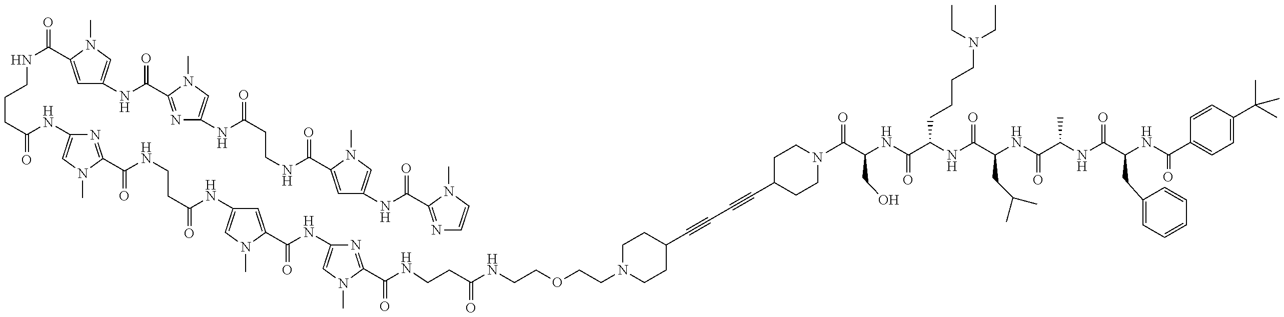 | 181 |
| 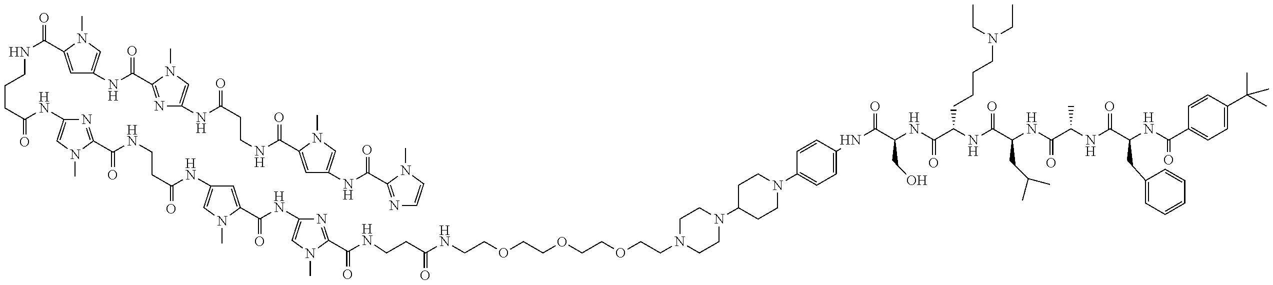 | 182 |
| 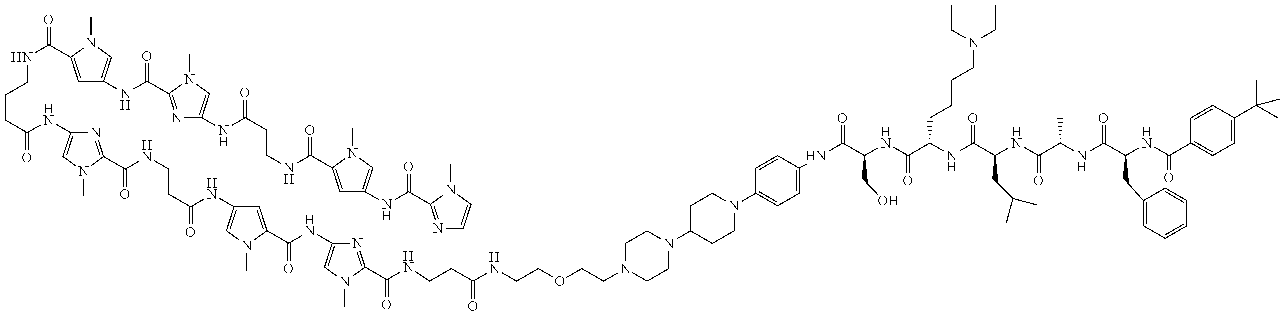 | 183 |
| 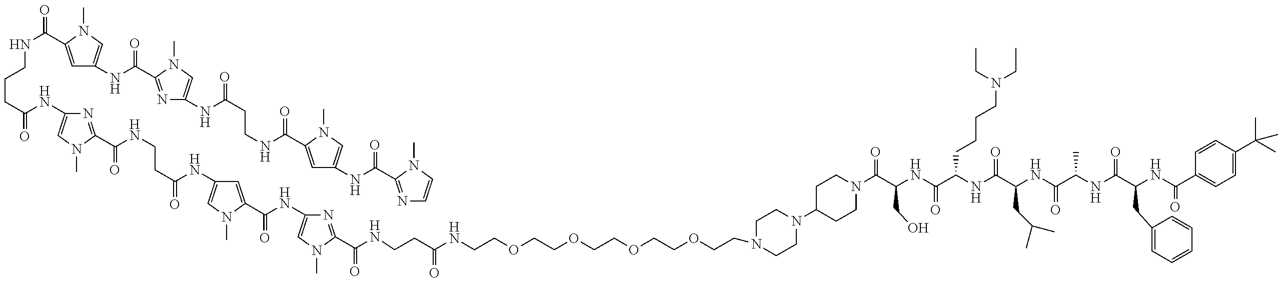 | 184 |
| 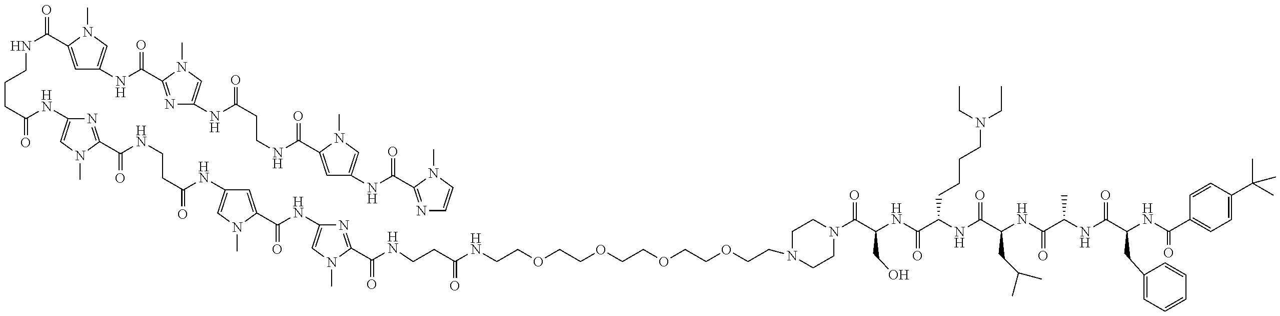 | 185 |
| 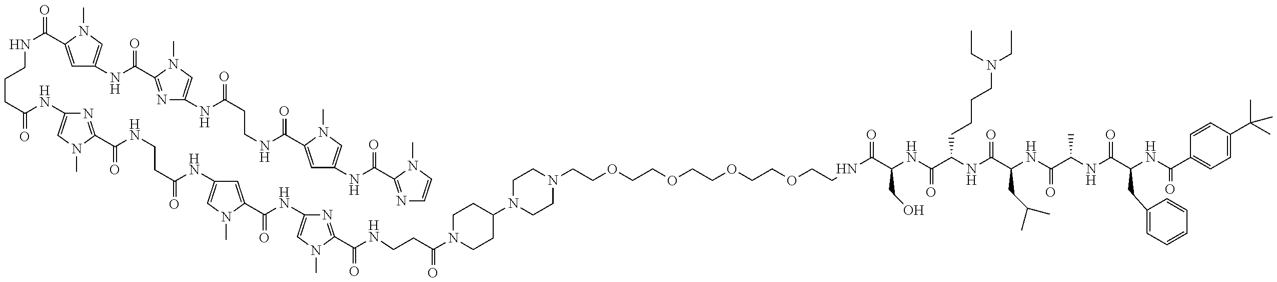 | 186 |
| 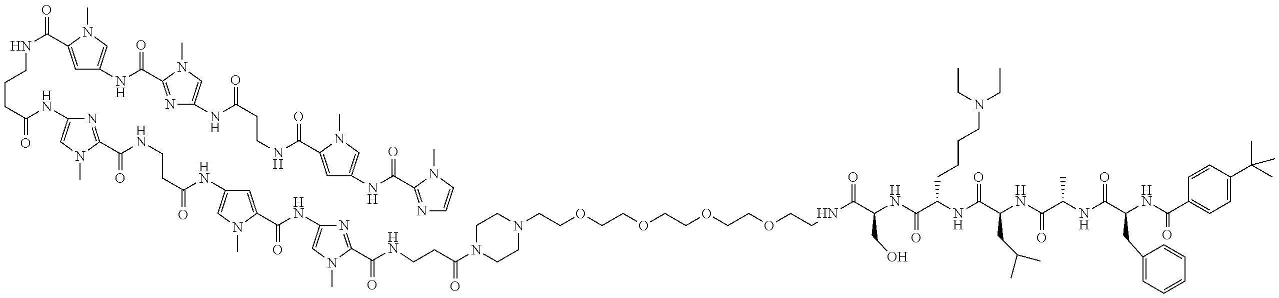 | 187 |
| 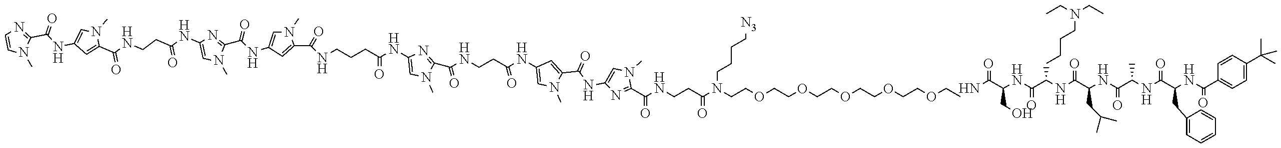 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCN(CCCCN=[N+]=[N-])C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | 189 |
| 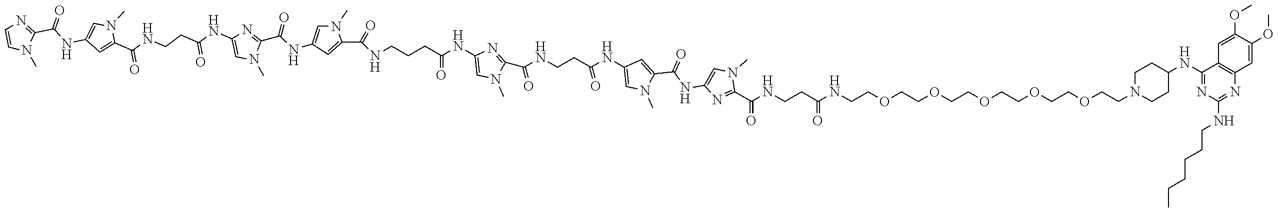 [SMILES: CCCCCCNc1nc(NC2CCN(CCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)CC2)c2cc(OC)c(OC)cc2n1] | 190 |
| 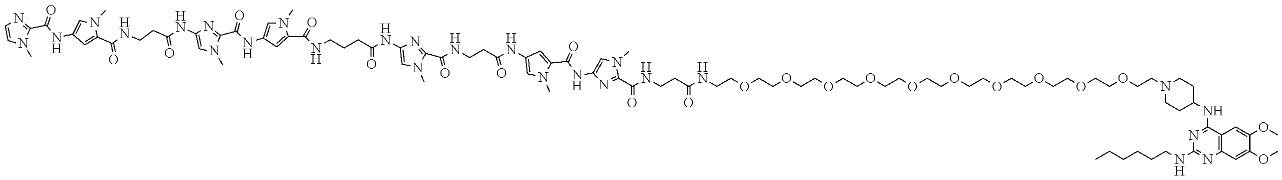 [SMILES: CCCCCCNc1nc(NC2CCN(CCOCCOCCOCCOCCOCCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)CC2)c2cc(OC)c(OC)cc2n1] | 191 |
| 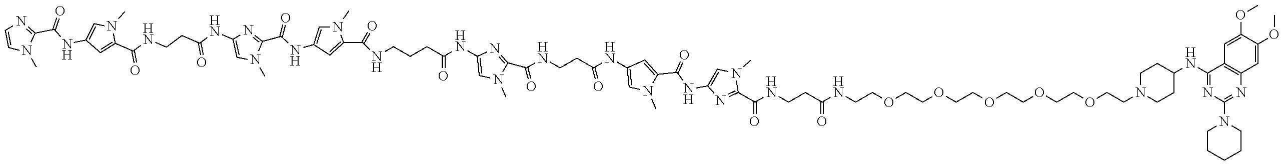 [SMILES: COc1cc2nc(N3CCCCC3)nc(NC3CCN(CCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)c2cc1OC] | 192 |
| 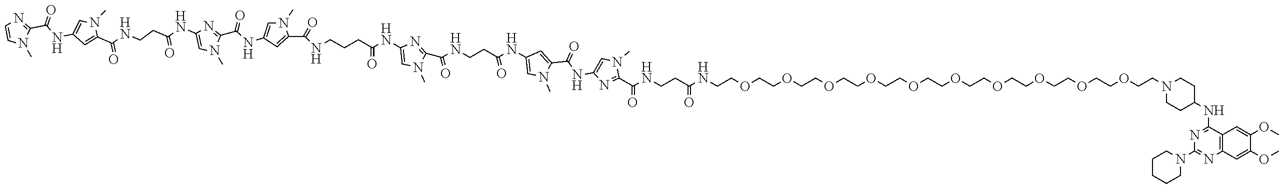 [SMILES: COc1cc2nc(N3CCCCC3)nc(NC3CCN(CCOCCOCCOCCOCCOCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)c2cc1OC] | 193 |
| 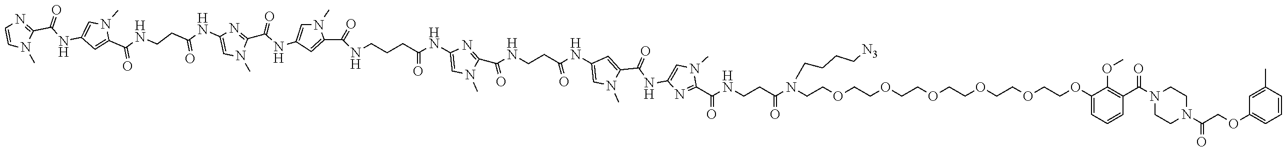 | 258 |
| 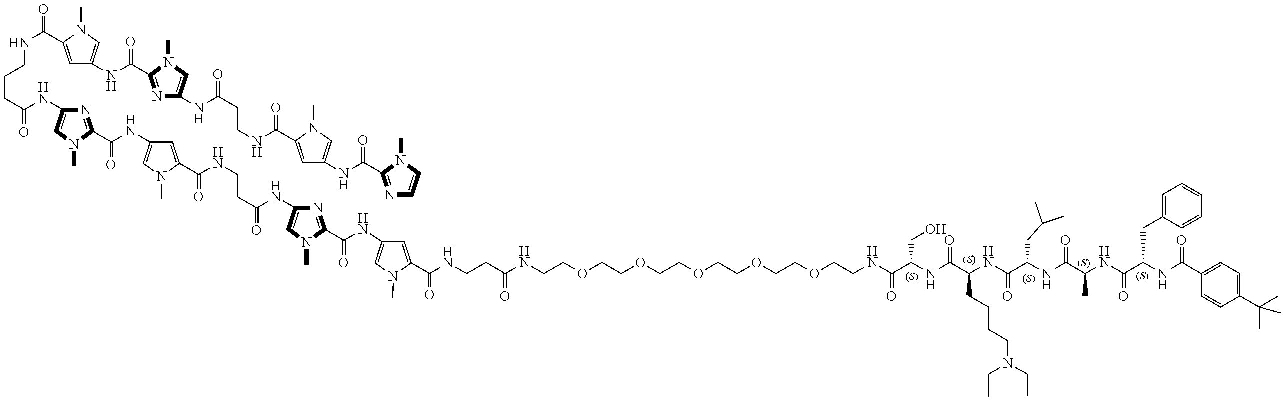 | 275 |
| 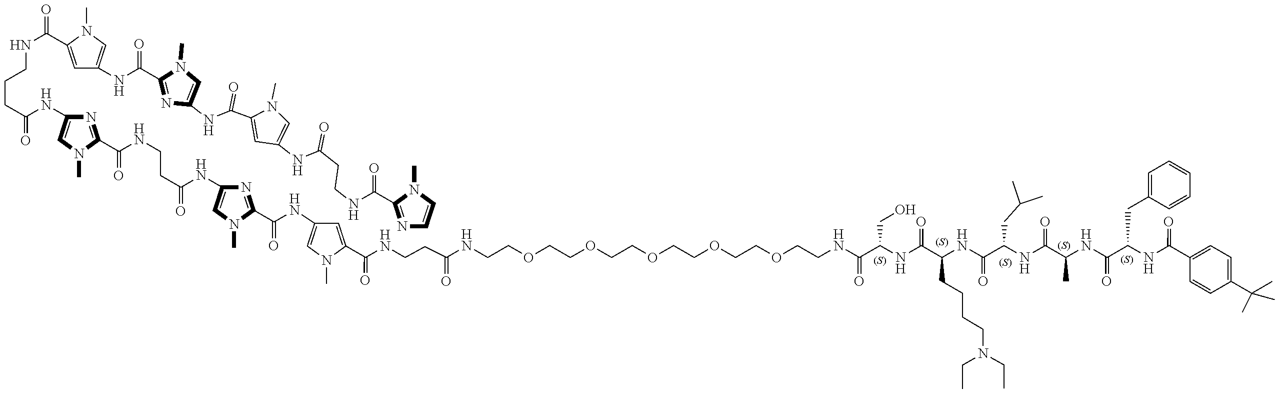 | 285 |

### Methods of Use

The present disclosure also relates to a method of modulating the transcription of dmpk, atxn1, atxn2, atxn3, cacna1a, atxn7, atxn8, atxn80s, ttbk2, tcf4, ppp2r2b, tbp, htt, jph3, ar, or atn1, the method comprising the step of contacting dmpk, atxn1, atxn2, atxn3, cacna1a, atxn7, atxn8, atxn80s, ttbk2, tcf4, ppp2r2b, tbp, htt, jph3, ar, or atn1 with a molecule as described herein.

The cell phenotype, cell proliferation, transcription of dmpk, atxn1, atxn2, atxn3, cacna1a, atxn7, atxn8, atxn80s, ttbk2, tcf4, ppp2r2b, tbp, htt, jph3, ar, or atn1, production of mRNA from transcription of dmpk, atxn1, atxn2, atxn3, cacna1a, atxn7, atxn8, atxn80s, ttbk2, tcf4, ppp2r2b, tbp, htt, jph3, ar, or atn1, translation of dmpk, atxn1, atxn2, atxn3, cacna1a, atxn7, ppp2r2b, tbp, htt, jph3, ar, or atn1, change in biochemical output produced by the protein coded by dmpk, atxn1, atxn2, atxn3, cacna1a, atxn7, atxn8, atxn80s, ttbk2, tcf4, ppp2r2b, tbp, htt, jph3, ar, or atn1, or noncovalent binding of the protein coded by dmpk, atxn1, atxn2, atxn3, cacna1a, atxn7, atxn8, atxn80s, ttbk2, tcf4, ppp2r2b, tbp, htt, jph3, ar, or atn1 with a natural binding partner may be monitored. Such methods may be modes of treatment of disease, biological assays, cellular assays, biochemical assays, or the like. In some embodiments, the gene is dmpk. In some embodiments, the gene is atxn1.

In some embodiments, the gene is atxn2. In some embodiments, the gene is atxn3. In some embodiments, the gene is cacna1a. In some embodiments, the gene is atxn7. In some embodiments, the gene is ppp2r2. In some embodiments, the gene is tbp. In some embodiments, the gene is htt. In some embodiments, the gene is jph3. In some embodiments, the gene is ar. In some embodiments, the gene is atn1. In some embodiments, the gene is atxn8. In some embodiments, the gene is atxn80s. In some embodiments, the gene is ttbk2. In some embodiments, the gene is tcf4. In some embodiments, the gene is htt.

Also provided herein is a method of treatment of a disease mediated by transcription of dmpk, atxn1, atxn2, atxn3, cacna1a, atxn7, atxn8, atxn80s, ttbk2, tcf4, ppp2r2b, tbp, htt, jph3, ar, or atn1 comprising the administration of a therapeutically effective amount of a molecule as disclosed herein, or a salt thereof, to a patient in need thereof.

In certain embodiments, the disease is chosen from DM1, spinocerebellar ataxia, Huntington's disease, a Huntington's disease-like syndrome, spinobulbar muscular atrophy, and dentatorubral-pallidoluysian atrophy.

In certain embodiments, the disease is chosen from DM1.

In certain embodiments, the disease is spinocerebellar ataxia. In certain embodiments, the spinocerebellar ataxia is chosen from SCA1, SCA2, SCA3, SCA6, SCA7, SCA12, and SCA17. In certain embodiments, the spinocerebellar ataxia is chosen from SCA1, SCA2, SCA3, SCA6, SCA7, and SCA17.

In certain embodiments, the disease is chosen from Huntington's disease and a Huntington's disease-like syndrome. In certain embodiments, the disease is chosen from Huntington's disease and Huntington's disease-like 2 syndrome.

In certain embodiments, the disease is spinobulbar muscular atrophy.

In certain embodiments, the disease is dentatorubral-pallidoluysian atrophy.

In certain embodiments, the disease is Fuchs' Endothelial Corneal Dystrophy (FECD).

Also provided herein is a molecule as disclosed herein for use as a medicament.

Also provided herein is a molecule as disclosed herein for use as a medicament for the treatment of a disease mediated by transcription of dmpk, atxn1, atxn2, atxn3, cacna1a, atxn7, atxn8, atxn80s, ttbk2, tcf4, ppp2r2b, tbp, htt, jph3, ar, or atn1.

Also provided is the use of a molecule as disclosed herein as a medicament.

Also provided is the use of a molecule as disclosed herein as a medicament for the treatment of a disease mediated by transcription of dmpk, atxn1, atxn2, atxn3, cacna1a, atxn7, atxn8, atxn80s, ttbk2, tcf4, ppp2r2b, tbp, htt, jph3, ar, or atn1.

Also provided is a molecule as disclosed herein for use in the manufacture of a medicament for the treatment of a disease mediated by transcription of dmpk, atxn1, atxn2, atxn3, cacna1a, atxn7, atxn8, atxn80s, ttbk2, tcf4, ppp2r2b, tbp, htt, jph3, ar, or atn1.

Also provided is the use of a molecule as disclosed herein for the treatment of a disease mediated by transcription of dmpk, atxn1, atxn2, atxn3, cacna1a, atxn7, atxn8, atxn80s, ttbk2, tcf4, ppp2r2b, tbp, htt, jph3, ar, or atn1.

Also provided herein is a method of modulation of transcription of dmpk, atxn1, atxn2, atxn3, cacna1a, atxn7, atxn8, atxn80s, ttbk2, tcf4, ppp2r2b, tbp, htt, jph3, ar, or atn1 comprising contacting dmpk, atxn1, atxn2, atxn3, cacna1a, atxn7, atxn8, atxn80s, ttbk2, tcf4, ppp2r2b, tbp, htt, jph3, ar, or atn1 with a molecule as disclosed herein, or a salt thereof.

Also provided herein is a method for achieving an effect in a patient comprising the administration of a therapeutically effective amount of a molecule as disclosed herein, or a salt thereof, to a patient, wherein the effect is chosen from muscular atrophy, ataxia, fasciculations, dementia, dysarthria, and dysphagia.

Also provided herein is a method for the prevention or treatment of a disease or condition mediated by or associated with the transcription of TCF4.

A method of modulation of the expression of the TCF4 comprising contacting TCF4 with a molecule described herein.

A method of treatment of a disease caused by transcription of TCF4 comprising the administration of a therapeutically effective amount of a molecule described herein to a patient in need thereof.

Some embodiments relate to a method of treatment of a disease caused by transcription of TCF4 comprising the administration of: a therapeutically effective amount of a molecule described herein; and another therapeutic agent.

Provided herein is a method for achieving an effect in a patient comprising the administration of a therapeutically effective amount of a molecule as disclosed herein, or a salt thereof, to a patient, wherein the effect is chosen from glare, blurred vision, pain or grittiness on cornea.

Certain molecules of the present disclosure may be effective for treatment of subjects whose genotype has 3 or more repeats of CAG or CTG. Certain molecules of the present disclosure may be effective for treatment of subjects whose genotype has 5 or more repeats of CAG or CTG. Certain molecules of the present disclosure may be effective for treatment of subjects whose genotype has 10 or more repeats of CAG or CTG. Certain molecules of the present disclosure may be effective for treatment of subjects whose genotype has 20 or more repeats of CAG or CTG. Certain molecules of the present disclosure may be effective for treatment of subjects whose genotype has 50 or more repeats of CAG or CTG. Certain molecules of the present disclosure may be effective for treatment of subjects whose genotype has 100 or more repeats of CAG or CTG. Certain molecules of the present disclosure may be effective for treatment of subjects whose genotype has 200 or more repeats of CAG or CTG. Certain compounds or molecules of the present disclosure may be effective for treatment of subjects whose genotype has 500 or more repeats of CAG or CTG.

Also provided is a method of modulation of a dmpk, atxn1, atxn2, atxn3, cacna1a, atxn7, atxn8, atxn80s, ttbk2, tcf4, ppp2r2b, tbp, htt, jph3, ar, or atn1-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for intravenous injection and/or infusion.

In certain embodiments, the oral pharmaceutical composition is chosen from a tablet and a capsule.

In certain embodiments, ex vivo methods of treatment are provided. Ex vivo methods typically include cells, organs, and/or tissues removed from the subject. The cells, organs and/or tissues can, for example, be incubated with the agent under appropriate conditions. The contacted cells, organs, and/or tissues are typically returned to the donor, placed in a recipient, or stored for future use. Thus, the compound is generally in a pharmaceutically acceptable carrier.

In certain embodiments, administration of the pharmaceutical composition modulates expression of the target gene within 6 hours of treatment. In certain embodiments, administration of the pharmaceutical composition modulates expression of dmpk, atxn1, atxn2, atxn3, cacna1a, atxn7, atxn8, atxn80s, ttbk2, tcf4, ppp2r2b, tbp, htt, jph3, ar, or atn1 within 24 hours of treatment. In certain embodiments, administration of the pharmaceutical composition modulates expression of dmpk, atxn1, atxn2, atxn3, cacna1a, atxn7, atxn8, atxn80s, ttbk2, tcf4, ppp2r2b, tbp, htt, jph3, ar, or atn1 within 72 hours of treatment.

In certain embodiments, administration of the pharmaceutical composition causes a 2-fold increase in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 5-fold increase in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 10-fold increase in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 20-fold increase in expression of dmpk, atxn1, atxn2, atxn3, cacna1a, atxn7, atxn8, atxn80s, ttbk2, tcf4, ppp2r2b, tbp, htt, jph3, ar, or atn1.

In certain embodiments, administration of the pharmaceutical composition causes a 20% decrease in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 50% decrease in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 80% decrease in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 90% decrease in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 95% decrease in expression of dmpk, atxn1, atxn2, atxn3, cacna1a, atxn7, atxn8, atxn80s, ttbk2, tcf4, ppp2r2b, tbp, htt, jph3, ar, or atn1. In certain embodiments, administration of the pharmaceutical composition causes a 99% decrease in expression of dmpk, atxn1, atxn2, atxn3, cacna1a, atxn7, atxn8, atxn80s, ttbk2, tcf4, ppp2r2b, tbp, htt, jph3, ar, or atn1.

In certain embodiments, administration of the pharmaceutical composition causes expression of dmpk, atxn1, atxn2, atxn3, cacna1a, atxn7, atxn8, atxn80s, ttbk2, tcf4, ppp2r2b, tbp, htt, jph3, ar, or atn1 to fall within 25% of the level of expression observed for healthy individuals. In certain embodiments, administration of the pharmaceutical composition causes expression of dmpk, atxn1, atxn2, atxn3, cacna1a, atxn7, atxn8, atxn80s, ttbk2, tcf4, ppp2r2b, tbp, htt, jph3, ar, or atn1 to fall within 50% of the level of expression observed for healthy individuals. In certain embodiments, administration of the pharmaceutical composition causes expression of dmpk, atxn1, atxn2, atxn3, cacna1a, atxn7, atxn8, atxn80s, ttbk2, tcf4, ppp2r2b, tbp, htt, jph3, ar, or atn1 to fall within 75% of the level of expression observed for healthy individuals. In certain embodiments, administration of the pharmaceutical composition causes expression of dmpk, atxn1, atxn2, atxn3, cacna1a, atxn7, atxn8, atxn80s, ttbk2, tcf4, ppp2r2b, tbp, htt, jph3, ar, or atn1 to fall within 90% of the level of expression observed for healthy individuals.

### Pharmaceutical Compositions and Administration

Also provided is a method of modulation of a dmpk, atxn1, atxn2, atxn3, cacna1a, atxn7, atxn8, atxn0s, ttbk2, tcf4, ppp2r2b, tbp, htt, jph3, ar, or atn1-mediated function in a subject comprising the administration of a therapeutically effective amount of a compound or molecule as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for intravenous injection or infusion.

In certain embodiments, the oral pharmaceutical composition is chosen from a tablet and a capsule.

In certain embodiments, ex vivo methods of treatment are provided. Ex vivo methods typically include cells, organs, or tissues removed from the subject. The cells, organs or tissues can, for example, be incubated with the agent under appropriate conditions. The contacted cells, organs, or tissues are typically returned to the donor, placed in a recipient, or stored for future use. Thus, the compound is generally in a pharmaceutically acceptable carrier.

### Abbreviations and Definitions

As used herein, the terms below have the meanings indicated.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene”, “heteroarylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

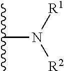

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

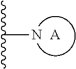

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

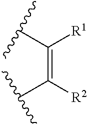

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

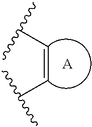

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

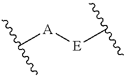

includes the substituent being oriented such that the A is attached at the leftmost attachment point of the molecule as well as the case in which A is attached at the rightmost attachment point of the molecule.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen Substituents, and chemical derivatives thereof. The aliphatic functionalities of linkable units can be provided, for example, by condensation of B-alanine or dimethylaminopropylamine during synthesis of the polyamide by methods well known in the art.

The term “linker” refers to a chain of at least 10 contiguous atoms. In certain embodiments, the linker contains no more than 20 non-hydrogen atoms. In certain embodiments, the linker contains no more than 40 non-hydrogen atoms. In certain embodiments, the linker contains no more than 60 non-hydrogen atoms. In certain embodiments, the linker contains atoms chosen from C, H, N, O, and S. In certain embodiments, every non-hydrogen atom is chemically bonded either to 2 neighboring atoms in the linker, or one neighboring atom in the linker and a terminus of the linker. In certain embodiments, the linker forms an amide bond with at least one of the two other groups to which it is attached. In certain embodiments, the linker forms an ester or ether bond with at least one of the two other groups to which it is attached. In certain embodiments, the linker forms a thioester or thioether bond with at least one of the two other groups to which it is attached. In certain embodiments, the linker forms a direct carbon-carbon bond with at least one of the two other groups to which it is attached. In certain embodiments, the linker forms an amine or amide bond with at least one of the two other groups to which it is attached. In certain embodiments, the linker comprises —(CH₂OCH₂)— units. In certain embodiments, the linker comprises —(CH(CH₃)OCH₂)— units. In certain embodiments, the linker comprises —(CH₂NR_{N}CH₂) units, for R_{N}═C₁₋₄alkyl. In certain embodiments, the linker comprises an arylene, cycloalkylene, or heterocycloalkylene moiety.

The term “spacer” refers to a chain of at least 5 contiguous atoms. In certain embodiments, the spacer contains no more than 10 non-hydrogen atoms. In certain embodiments, the spacer contains atoms chosen from C, H, N, O, and S. In certain embodiments, the spacer forms amide bonds with the two other groups to which it is attached. In certain embodiments, the spacer comprises —(CH₂OCH₂)— units. In certain embodiments, the spacer comprises —(CH₂NR_{N}CH₂)— units, for R_{N}═C₁₄alkyl. In certain embodiments, the spacer contains at least one positive charge at physiological pH.

The term “turn component” refers to a chain of about 4 to 10 contiguous atoms. In certain embodiments, the turn component contains atoms chosen from C, H, N, O, and S. In certain embodiments, the turn component forms amide bonds with the two other groups to which it is attached. In certain embodiments, the turn component contains at least one positive charge at physiological pH.

The terms “nucleic acid and “nucleotide” refer to ribonucleotide and deoxyribonucleotide, and analogs thereof, well known in the art.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amide,” as used herein, alone in combination, refers to —C(O)NRR′, wherein R and R are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. Amides may be formed by direct condensation of carboxylic acids with amines, or by using acid chlorides. In addition, coupling reagents are known in the art, including carbodiimide-based compounds such as DCC and EDCI.

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl. The term “arylene” embraces aromatic groups such as phenylene, naphthylene, anthracenylene, and phenanthrylene.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzindolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include tetrahydroisoquinoline, aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds or molecules of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R are independently chosen from hydrogen and lower alkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethylsilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with”.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds or molecules disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds or molecules can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds or molecules of particular stereochemistry are either commercially available or can be made and resolved by techniques known in the art. Additionally, the compounds or molecules disclosed herein may exist as geometric isomers. The present disclosure includes all cis, trans, syn, anti, entgegen (E), and zusammen (Z) isomers as well as the appropriate mixtures thereof. Additionally, compounds or molecules may exist as tautomers; all tautomeric isomers are provided by this disclosure. Additionally, the compounds or molecules disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds or molecules (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound or molecule that is made more active in vivo. Certain compounds or molecules disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The term “contacting” refers to bringing the compound (e.g. a transcription molecular molecule of the present disclosure) into proximity of the desired target gene. The contacting may result in the binding to or result in a conformational change of the target moiety.

The compounds or molecules disclosed herein can exist as therapeutically acceptable salts. The present disclosure includes compounds or molecules listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound or molecule in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

Basic addition salts can be prepared during the final isolation and purification of the compounds or molecules by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

The amount of active ingredient that may be combined with the carrier materials to produce a single dosage form will vary depending upon the host treated and the particular mode of administration.

The compounds or molecules can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

### Combinations and Combination Therapy

In certain instances, it may be appropriate to administer at least one of the compounds or molecules described herein (or a pharmaceutically acceptable salt thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating disorders described herein in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of disorders. The disorders can be associated with the expression of defective genes such as dmpk, atxn1, atxn2, atxn3, cacna1a, atxn7, ppp2r2b, tbp, htt, jph3, ar, atn, and gene encoding TCF4.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

### List of Abbreviation

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC·HCl=EDCI·HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(O); Pd₂(dba)₃=tris(dibenzylideneacetone)-dipalladium(O); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSCl=TBDMSCl=tert-butyldimethylchlorosilane; TEA=Et₃N=triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

General Synthetic Methods for Preparing Compounds

In general, polyamides of the present disclosure may be synthesized by solid supported synthetic methods, using compounds such as Boc-protected straight chain aliphatic and heteroaromatic amino acids, and alkylated derivatives thereof, which are cleaved from the support by aminolysis, deprotected (e.g., with sodium thiophenoxide), and purified by reverse-phase HPLC, as well known in the art. The identity and purity of the polyamides may be verified using any of a variety of analytical techniques available to one skilled in the art such as ¹H-NMR, analytical HPLC, or mass spectrometry.

The following scheme can be used to practice the present disclosure:

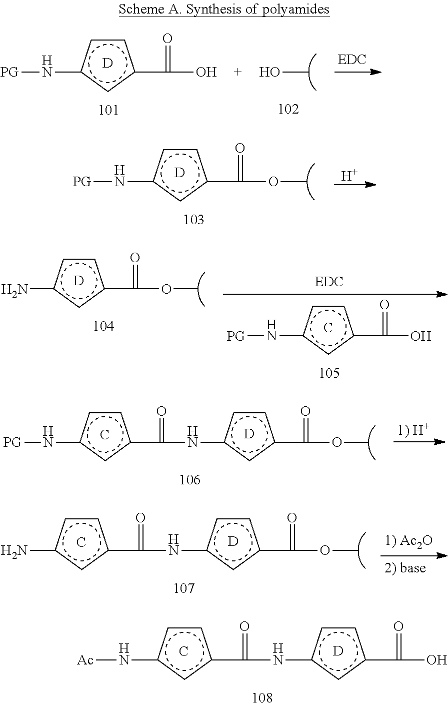

The compounds disclosed herein can be synthesized using Scheme A. For clarity and compactness, the scheme depicts the synthesis of a diamide comprising subunits “C” and “D”, both of which are represented as unspecified five-membered rings having amino and carboxy moieties. The amino group of subunit “D” is protected with a protecting group “PG” such as a Boc or CBz carbamate to give 101. The free carboxylic acid is then reacted with a solid support, using a coupling reagent such as EDC, to give the supported compound 103. Removal of PG under acidic conditions gives the free amine 104, which is coupled with the nitrogen-protected carboxylic acid 105 to give amide 106. Removal of PG under acidic conditions gives the free amine 107. In this example, the free amine is reacted with acetic anhydride to form an acetamide (not shown. The molecule is then cleaved from the solid support under basic conditions to give carboxylic acid 108. Methods for attachment of the linker L and recruiting moiety X are disclosed below.

The person of skill will appreciate that many variations of the above scheme are available to provide a wide range of compounds:
1) The sequence 104-106-107 can be repeated as often as desired, in order to form longer polyamine sequences.
2) A variety of amino heterocycle carboxylic acids can be used, to form different subunits. Table 4, while not intended to be limiting, provides several heterocycle amino acids that are contemplated for the synthesis of the compounds in this disclosure. Carbamate protecting groups PG can be incorporated using techniques that are well established in the art.

TABLE 4
Heterocyclic amino acids.
Structure

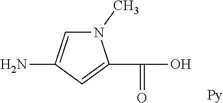

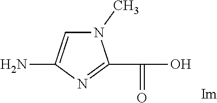

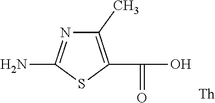

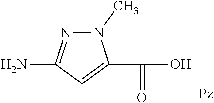

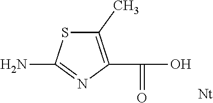

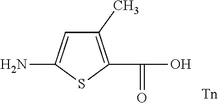

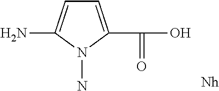

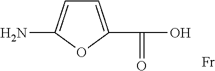

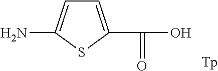

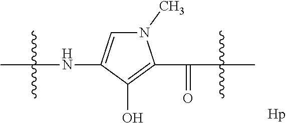 [SMILES: *Nc1cn(C)c(C(*)=O)c1O ; "Hp"]

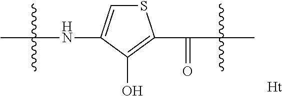 [SMILES: *Nc1csc(C(*)=O)c1O ; "Ht"]

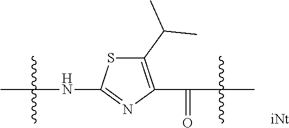 [SMILES: *Nc1nc(C(*)=O)c(C(C)C)s1 ; "iNt"]

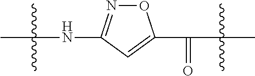 [SMILES: *Nc1cc(C(*)=O)on1]

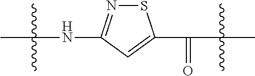 [SMILES: *Nc1cc(C(*)=O)sn1]

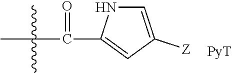

(Z is H, C₁₋₆ alkyl, amine, or
halogen)

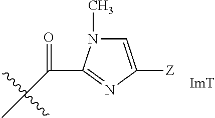

(Z is H, C₁₋₆ alkyl, amine, or
halogen)

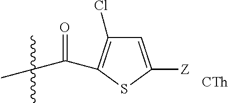

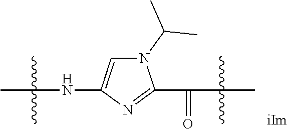 [SMILES: *Nc1cn(C(C)C)c(C(*)=O)n1 ; "iIm"]

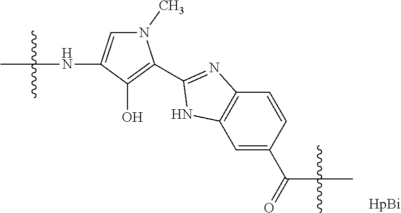 [SMILES: *Nc1cn(C)c(-c2nc3ccc(C(*)=O)cc3[nH]2)c1O]

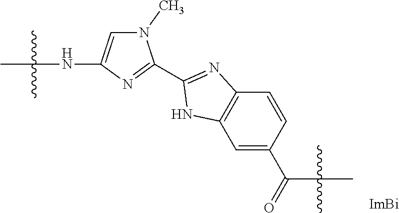 [SMILES: *Nc1cn(C)c(-c2nc3ccc(C(*)=O)cc3[nH]2)n1]

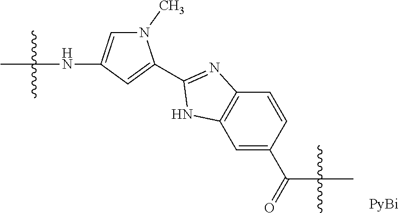 [SMILES: *Nc1cc(-c2nc3ccc(C(*)=O)cc3[nH]2)n(C)c1]

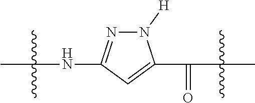 [SMILES: *Nc1cc(C(*)=O)[nH]n1]

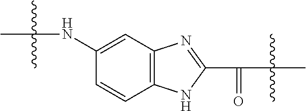 [SMILES: *Nc1ccc2[nH]c(C(*)=O)nc2c1]

3) Hydroxy-containing heterocyclic amino acids can be incorporated into Scheme B as their TBS ethers. While not intended to be limiting, Scheme B provides the synthesis of TBS-protected heterocyclic amino acids contemplated for the synthesis of the molecules in this disclosure.

### Scheme B. Synthesis of TBS-Protected Heterocyclic Amino Acids

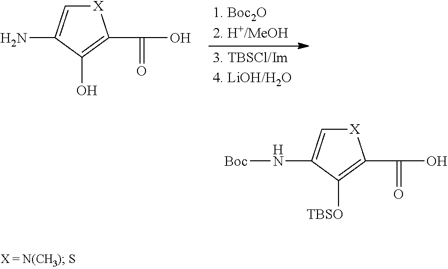 [0641] 4) Aliphatic amino acids can be used in the above synthesis for the formation of spacer units “W” and subunits for recognition of DNA nucleotides. Table 5, while not intended to be limiting, provides several aliphatic amino acids contemplated for the synthesis of the or molecules in this disclosure.

TABLE 5
Aliphatic amino acids
Structure

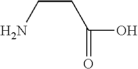

beta-alanine (β)

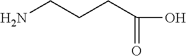

gamma-aminobutyric acid (“gAB” or γ)

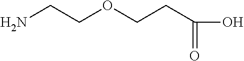

3-(2-aminoethoxy)propanoic acid

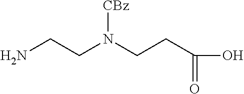

3-((2-aminoethyl)(2-oxo-2-phenyl-1λ²-ethyl)amino)propanoic acid

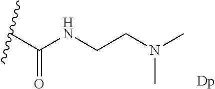 [SMILES: *C(=O)NCCN(C)C ; "Dp"]

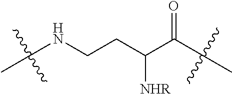

(R if H, C₁₋₆ alkyl)

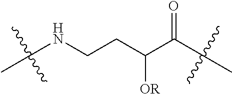

(R is H, C₁₋₆ alkyl, aryl, or heteroaryl)

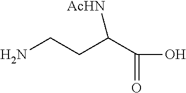 [SMILES: CC(=O)NC(CCN)C(=O)O]

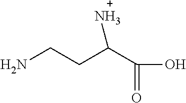

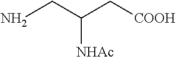 [SMILES: CC(=O)NC(CN)CC(=O)O]

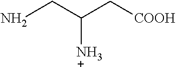

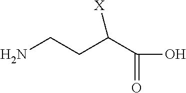

X is F or OH

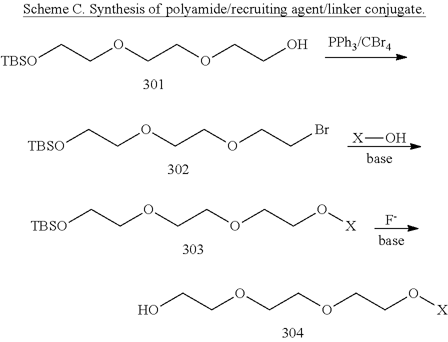

Attachment of the linker L and recruiting moiety X can be accomplished with the methods disclosed in Scheme III, which uses a triethylene glycol moiety for the linker L. The mono-TBS ether of triethylene glycol 301 is converted to the bromo compound 302 under Mitsunobu conditions. The recruiting moiety X is attached by displacement of the bromine with a hydroxyl moiety, affording ether 303. The TBS group is then removed by treatment with fluoride, to provide alcohol 304, which will be suitable for coupling with the polyamide moiety. Other methods will be apparent to the person of skill in the art for inclusion of alternate linkers L, including but not limited to propylene glycol or polyamine linkers, or alternate points of attachment of the recruiting moiety X, including but not limited to the use of amines and thiols.

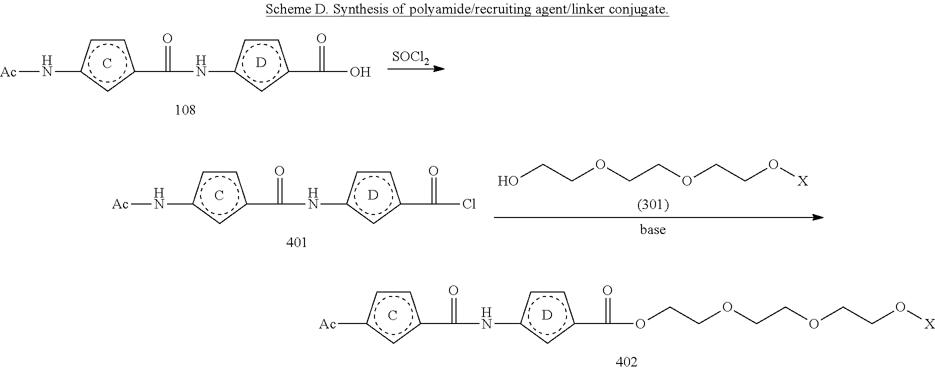

Synthesis of the X-L-Y molecule can be completed with the methods set forth in Scheme D. Carboxylic acid 108 is converted to the acid chloride 401. Reaction with the alcohol functionality of 301 under basic conditions provides the coupled product 402. Other methods will be apparent to the person of skill in the art for performing the coupling procedure, including but not limited to the use of carbodiimide reagents. For instance, the amide coupling reagents can be used, but not limited to, are carbodiimides such as dicyclohexylcarbodiimide (DCC), diisopropylcarbodiimide (DIC), ethyl-(N′,N′-dimethylamino)propylcarbodiimide hydrochloride (EDC), in combination with reagents such as 1-hydroxybenzotriazole (HOBt), 4-(N,N-dimethylamino)pyridine (DMAP) and diisopropylethylamine (DIEA). Other reagents are also often used depending the actual coupling reactions are (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), Bis(2-oxo-3-oxazolidinyl)phosphinic chloride (BOP—Cl), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), O-(6-Chlorobenzotriazol-1-yl)-N,N,N′,N′-tetramethyluronium hexafluorophosphate (HCTU), Carbonyldiimidazole (CDI), and N,N,N′,N′-Tetramethylchloroformamidinium Hexafluorophosphate (TCFH).

### Attaching Protein Binding Molecules to Oligomeric Backbone

Generally the oligomeric backbone is functionalized to adapt to the type of chemical reactions can be performed to link the oligomers to the attaching position in protein binding moieties. The type reactions are suitable but not limited to, are amide coupling reactions, ether formation reactions (O-alkylation reactions), amine formation reactions (N-alkylation reactions), and sometimes carbon-carbon coupling reactions. The general reactions used to link oligomers and protein binders are shown in below. The compounds and structures shown in Table 2 can be attached to the oligomeric backbone described herein at any position that is chemically feasible while not interfering with the hydrogen bond between the compound and the regulatory protein.

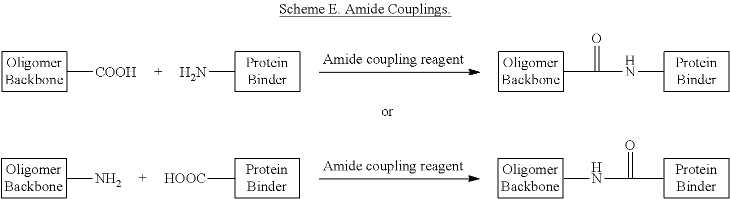

Either the oligomer or the protein binder can be functionalized to have a carboxylic acid and the other coupling counterpart being functionalized with an amino group so the moieties can be conjugated together mediated by amide coupling reagents. The amide coupling reagents can be used, but not limited to, are carbodiimides such as dicyclohexylcarbodiimide (DCC), diisopropylcarbodiimide (DIC), ethyl-(N′,N′-dimethylamino)propylcarbodiimide hydrochloride (EDC), in combination with reagents such as 1-hydroxybenzotriazole (HOBt), 4-(N,N-dimethylamino)pyridine (DMAP) and diisopropylethylamine (DIEA). Other reagents are also often used depending the actual coupling reactions are (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), Bis(2-oxo-3-oxazolidinyl)phosphinic chloride (BOP—Cl), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), O-(6-Chlorobenzotriazol-1-yl)-N,N,N′,N′-tetramethyluronium hexafluorophosphate (HCTU), Carbonyldiimidazole (CDI), and N,N,N′,N′-Tetramethylchloroformamidinium Hexafluorophosphate (TCFH).

In one aspect, the molecules of the present disclosure comprises a cell-penetrating ligand moiety. The cell-penetrating ligand moiety serves to facilitate transport of the compound across cell membranes. In certain embodiments, the cell-penetrating ligand moiety is a polypeptide. Several peptide sequences can facilitate passage into the cell, including polycationic sequences such as poly-R; arginine-rich sequences interspersed with spacers such as (RXR)ₙ (X=6-aminohexanoic acid) and (RXRRBR)ₙ (B=beta-alanine); sequences derived from the Penetratin peptide; and sequences derived from the PNA/PMO internalization peptide (Pip). The Pip5 series is characterized by the sequence ILFQY.

In certain embodiments, the cell-penetrating polypeptide comprises an N-terminal cationic sequence H₂N—(R)ₙ—CO—, with n=5-10, inclusive. In certain embodiments, the N-terminal cationic sequence contains 1, 2, or 3 substitutions of R for amino acid resides independently chosen from beta-alanine and 6-aminohexanoic acid.

In certain embodiments, the cell-penetrating polypeptide comprises the ILFQY sequence. In certain embodiments, the cell-penetrating polypeptide comprises the QFLY sequence. In certain embodiments, the cell-penetrating polypeptide comprises the QFL sequence.

In certain embodiments, the cell-penetrating polypeptide comprises a C-terminal cationic sequence —HN—(R)ₙ—COOH, with n=5-10, inclusive. In certain embodiments, the C-terminal cationic sequence contains 1, 2, or 3 substitutions of R for amino acid resides independently chosen from beta-alanine and 6-aminohexanoic acid. In certain embodiments, the C-terminal cationic sequence is substituted at every other position with an amino acid residue independently chosen from beta-alanine and 6-aminohexanoic acid. In certain embodiments, the C-terminal cationic sequence is —HN—RXRBRXRB—COOH.

TABLE 6
Cell-penetrating peptides.

| SEQ ID NO. | Sequence |
|---|---|
| SEQ ID NO. 1 | GRKKRRQRRRPPQ |
| SEQ ID NO. 2 | RQIKIWFQNRRMKWKK |
| SEQ ID NO. 3 | KLALKLALKALKAALKLA |
| SEQ ID NO. 4 | GWTLNS/AGYLLGKINLKALAALAKKIL |
| SEQ ID NO. 5 | NAKTRRHERRRKLAIER |
| SEQ ID NO. 6 | RRRRRRRR |
| SEQ ID NO. 7 | RRRRRRRRR |
| SEQ ID NO. 8 | GALFLGFLGAAGSTMGA |
| SEQ ID NO. 9 | KETWWETWWTEWSQPKKKRKV |
| SEQ ID NO. 10 | LLIILRRRIRKQAHAHSK |
| SEQ ID NO. 11 | YTAIAWVKAFIRKLRK |
| SEQ ID NO. 12 | IAWVKAFIRKLRKGPLG |
| SEQ ID NO. 13 | MVTVLFRRLRIRRACGPPRVRV |
| SEQ ID NO. 14 | GLWRALWRLLRSLWRLLWRA |
| SEQ ID NO. 15 | RRRRRRR QIKIWFQNRRMKWKKGG |
| SEQ ID NO. 16 | RXRRXRRXRIKILFQNRRMKWKK |
| SEQ ID NO. 17 | RXRRXRRXRIdKILFQNdRRMKWHKB |
| SEQ ID NO. 18 | RXRRXRRXRIHILFQNdRRMKWHKB |
| SEQ ID NO. 19 | RXRRBRRXRILFQYRXRBRXRB |
| SEQ ID NO. 20 | RXRRBRRXRILFQYRXRXRXRB |
| SEQ ID NO. 21 | RXRRXRILFQYRXRRXR |
| SEQ ID NO. 22 | RBRRXRRBRILFQYRBRXRBRB |
| SEQ ID NO. 23 | RBRRXRRBRILFQYRXRBRXRB |
| SEQ ID NO. 24 | RBRRXRRBRILFQYRXRRXRB |
| SEQ ID NO. 25 | RBRRXRRBRILFQYRXRBRXB |
| SEQ ID NO. 26 | RXRRBRRXRILFQYRXRRXRB |
| SEQ ID NO. 27 | RXRRBRRXRILFQYRXRBRXB |
| SEQ ID NO. 28 | RXRRBRRXRYQFLIRXRBRXRB |
| SEQ ID NO. 29 | RXRRBRRXRIQFLIRXRBRXRB |
| SEQ ID NO. 30 | RXRRBRRXRQFLIRXRBRXRB |
| SEQ ID NO. 31 | RXRRBRRXRQFLRXRBRXRB |
| SEQ ID NO. 32 | RXRRBRRXYRFLIRXRBRXRB |
| SEQ ID NO. 33 | RXRRBRRXRFQILYRXRBRXRB |
| SEQ ID NO. 34 | RXRRBRRXYRFRLIXRBRXRB |
| SEQ ID NO. 35 | RXRRBRRXILFRYRXRBRXRB |
| SEQ ID NO. 36 | Ac-RRLSYSRRRFXBpgG |
| SEQ ID NO. 37 | Ac-RRLSYSRRRFPFVYLIXBpgG |

Ac=acetyl; Bpg=L-bis-homopropargylglycine=

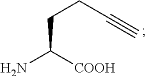

B=beta-alanine; X=6-aminohexanoic acid; dK/dR=corresponding D-amino acid.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

The following examples are intended to illustrate but not limit the disclosed embodiments. Scheme A describes the steps involved for preparing the polyamide, attaching the polyamide to the oligomeric backbone, and then attaching the ligand to the other end of the oligomeric backbone. The transcription modulator molecule such as those listed in Table 3 below can be prepared using the synthesis.

Synthesis of Representative Ligands

### Example 1. Synthesis of N-(3-[[5-chloro-4-(1H-indol-3-yl)pyrimidin-2-yl]amino]phenyl)-4-[(2E)-4-(methylamino)but-2-enamido]benzamide (INT-10)

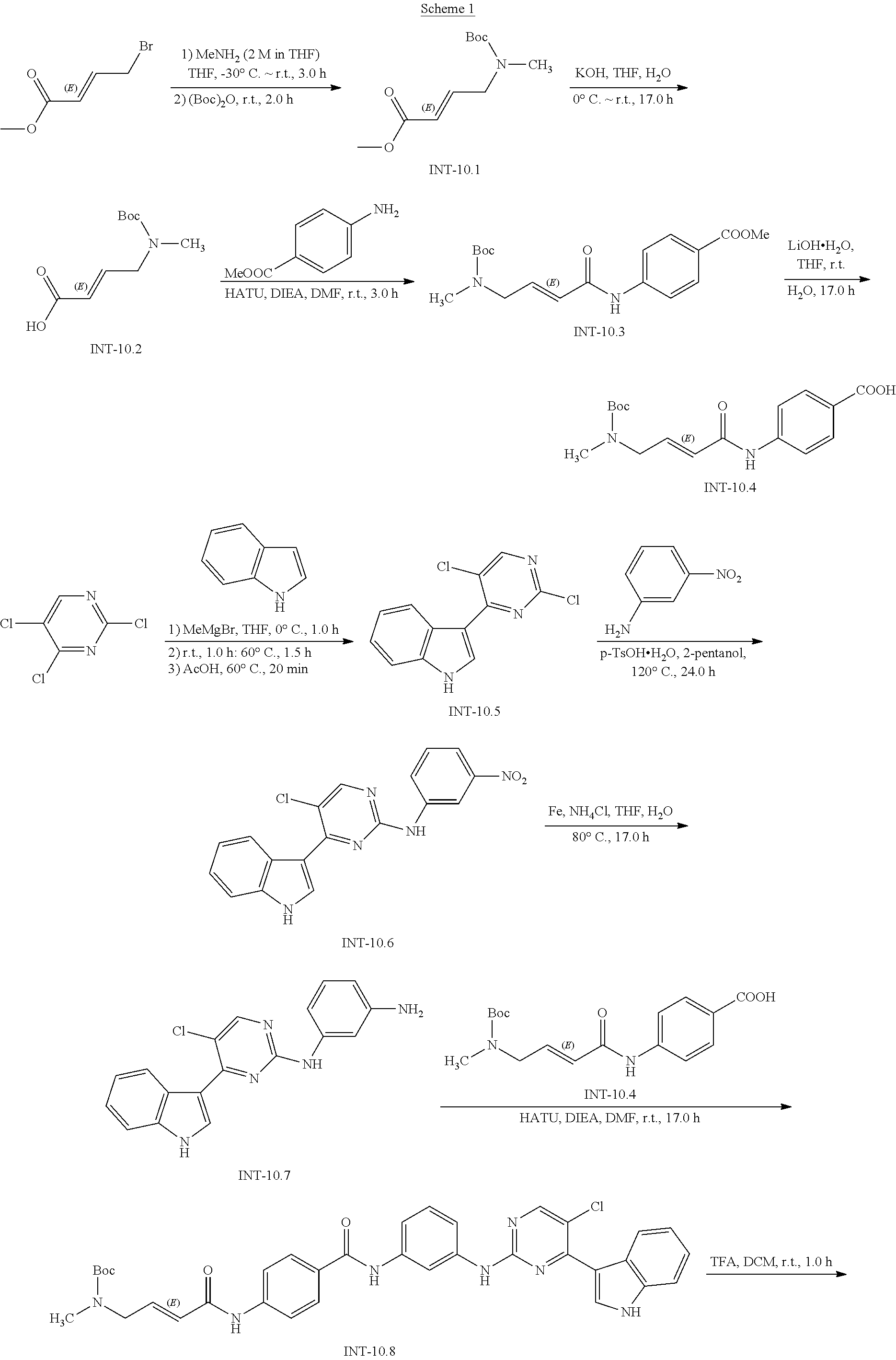

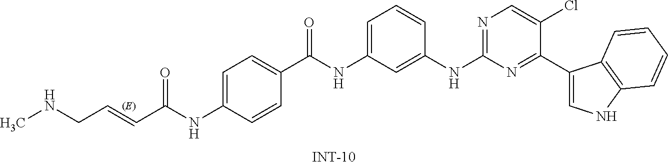

### Step: Synthesis of methyl (2E)-4-[(tert-butoxycarbonyl)(methyl)amino]but-2-enoate

Cooled a 3-neck-250 mL flask to −15 to −25° C. Charged an addition funnel with methyl (2E)-4-bromobut-2-enoate (5.00 g, 27.93 mmol, 1.00 equiv) in THF (25.00 mL). Charged a second addition funnel with methylamine (2M in THF) (13.79 mL, 2.50 eq). Add both solutions dropwise and simultaneously over 30 min maintaining the pot temperature at to −30° C. Warm the mixture to room temperature and hold for 3.0 h. Then the reaction mixture was cooled to −60 to −65° C. TEA (14.13 g, 139.66 mmol, 5.00 equiv) was added and a solution of (Boc)₂O (24.38 g, 111.73 mmol, 4.00 equiv) in THF (50.00 mL) was added dropwise over 1.0 h. Hold the mixture 2.0 h while allowing it to warm to room temperature. Check that the reaction is complete by TLC (9:1 heptane:EtOAc). The precipitates are filtered off and the mixture was concentrated to oil. The oil is re-dissolved in CH₂Cl₂ (50 mL), washed consecutively with water (25 mL), 1 N HCl (25 mL) and water (2×25 mL). The organic layer is dried over sodium sulfate (10 g) for 10 mins. The mixture is filtered and concentrated. Methyl (2E)-4-[(tert-butoxycarbonyl)(methyl)amino]but-2-enoate (6.00 g, 92.75% yield) was obtained as a yellow oil. LC/MS: mass calcd. For C₁₁H₁₉NO₄: 229.13, found: 247.15 [M+H₂O]⁺.

### Step 2: Synthesis of (2E)-4-[(tert-butoxycarbonyl)(methyl)amino]but-2-enoic acid

To a stirred solution of methyl (2E)-4-[(tert-butoxycarbonyl)(methyl)amino]but-2-enoate (6.00 g, 26.17 mmol, 1.00 equiv) in THF (40.00 mL) and H₂O (40.00 mL) was added KOH (5.87 g, 104.62 mmol, 4.00 equiv) at 0° C. The resulting mixture was stirred for 17.0 h at room temperature. The resulting mixture was concentrated under vacuum. The residue was dissolved in water (20 mL). The mixture was acidified to pH=4˜5 with 2M HCl. The resulting mixture was extracted with EA (3×50 mL). The combined organic layers were washed with aqueous NaCl (40 mL), dried over anhydrous Na₂SO₄. After filtration, the filtrate was concentrated under vacuum. (2E)-4-[(tert-butoxycarbonyl)(methyl)amino]but-2-enoic acid (4.50 g, 79.89% yield) was obtained as yellow oil. LC/MS: mass calcd. For C₁₀H₁₇NO₄: 215.12, found: 238.10 [M+Na]⁺.

### Step 3: Synthesis of methyl 4-[(2E)-4-[(tert-butoxycarbonyl)(methyl)amino]but-2-enamido]benzoate

To a stirred solution of (2E)-4-[(tert-butoxycarbonyl)(methyl)amino]but-2-enoic acid (4.50 g, 20.91 mmol, 1.00 equiv) in DMF (60.00 mL) was added HATU (9.54 g, 25.09 mmol, 1.20 equiv), DIEA (8.11 g, 62.72 mmol, 3.00 equiv) and methyl 4-aminobenzoate (3.16 g, 20.91 mmol, 1.00 equiv) at room temperature. The resulting mixture was stirred for 3.0 h at room temperature. The reaction was quenched with water/ice (150 mL) at 0° C.

The resulting mixture was extracted with EA (3×200 mL). The combined organic layers were washed with aqueous NaCl (80 mL), dried over anhydrous Na₂SO₄. After filtration, the filtrate was concentrated under vacuum.
The residue was purified by silica gel column chromatography, eluted with PE/EA (3:1) to afford methyl 4-[(2E)-4-[(tert-butoxycarbonyl)(methyl)amino]but-2-enamido]benzoate (2.00 g, 27.46% yield) as a yellow solid. LC/MS: mass calcd. For C₁₈H₂₄N₂O₅: 348.17, found: 371.05 [M+Na]⁺.

### Step 4: Synthesis of 4-[(2E)-4-[(tert-butoxycarbonyl)(methyl)amino]but-2-enamido]benzoic acid

To a stirred solution of methyl 4-[(2E)-4-[(tert-butoxycarbonyl)(methyl)amino] but-2-enamido]benzoate (1.00 g, 2.87 mmol, 1.00 equiv) in THF (10.00 mL) and H₂O (5.00 mL) was added LiOH·H₂O (361.34 mg, 8.61 mmol, 3.00 equiv) in portions at room temperature.

The resulting mixture was stirred for 17.0 h at room temperature.
The resulting mixture was concentrated under vacuum. Then the residue was dissolved in water (4 mL).
The mixture was neutralized to pH 7 with 2N HCl. The precipitated solids were collected by filtration and washed with H₂O (3×5 mL), dried under vacuum. 4-[(2E)-4-[(tert-butoxycarbonyl)(methyl) amino]but-2-enamido] benzoic acid (670.00 mg, 63.87% yield) was obtained as a light yellow solid. LC/MS: mass calcd. For C₁₇H₂₂N₂O₅: 334.15, found: 335.15 [M+H]⁺.

### Step 5: Synthesis of 3-(2,5-dichloropyrimidin-4-yl)-1H-indole

Methylmagnesium bromide (3.2 M in 2-methyltetrahydrofuran, 9.84 mL, 85.41 mmol, 2.00 equiv) was added dropwise over 10 min to a solution of indole (10.00 g, 117.15 mmol, 2.75 equiv) in THF (200.00 mL) at 0° C. The solution was stirred at 0-2° C. for 30 minutes. 2,4,5-Trichloropyrimidine (7.83 g, 42.68 mmol, 1.00 equiv) was added dropwise. The ice bath was removed and the solution was stirred at ambient temperature for 1.0 h. The temperature was elevated to 60° C. and the mixture was stirred at 60° C. for 1.5 h. The mixture was cooled to 25° C. and acetic acid (4.94 mL, 86.4 mmol, 2.00 equiv) was added dropwise. Water (80 mL) and THF (16 mL) were added and the mixture was stirred for 20 min at 60° C., resulting in a biphasic solution. The layers were partitioned and heptane (80 mL) was added to the organic solution, resulting in the crystallization of a solid. The solid was collected by filtration, washed with heptane (16 mL) and dried in a vacuum oven to yield 3-(2,5-dichloropyrimidin-4-yl)-1H-indole as yellow solid (5.10 g, 21.45% yield). LC/MS: mass calcd. For C₁₂H₇Cl₂N₃:263.00, found: 264.05 [M+H]⁺.

### Step 6: Synthesis of 5-chloro-4-(1H-indol-3-yl)-N-(3-nitrophenyl)pyrimidin-2-amine

Into a 250 mL round-bottom flask was added 3-(2,5-dichloropyrimidin-4-yl)-1H-indole (5.10 g, 19.31 mmol, 1.00 equiv), p-Toluenesulfonic acid (6.65 g, 38.62 mmol, 2.00 equiv) and 3-nitroaniline (2.67 g, 19.33 mmol, 1.00 equiv) at room temperature. The resulting mixture was stirred for 24.0 h at 120° C. under nitrogen atmosphere. The reaction mixture was evaporated to dryness, re-dissolved in DCM (100 mL) and MeOH (10 mL) and washed sequentially with saturated NaHCO₃ (50 mL), water (50 mL) and saturated brine (50 mL). The organic layer was dried over Na₂SO₄. The solid was filtered out and filtrate was evaporated to afford the crude product. The resultant solid was triturated with DCM/MeOH (9:1), filtered and washed with DCM to give 5-chloro-4-(1H-indol-3-yl)-N-(3-nitrophenyl)pyrimidin-2-amine as light yellow solid (4.90 g, 96.00% yield). LC/MS: mass calcd. For C₁₈H₁₂ClN₅O₂: 365.07, found: 366.15 [M+H]⁺.

### Step 7: Synthesis of N1-[5-chloro-4-(1H-indol-3-yl)pyrimidin-2-yl]benzene-1,3-diamine

To a solution of 5-chloro-4-(1H-indol-3-yl)-N-(3-nitrophenyl)pyrimidin-2-amine (4.90 g, 13.40 mmol, 1.00 equiv) in 40.00 mL THF and 40.00 mL H₂O was added Fe (11.22 g, 200.94 mmol, 15.00 equiv) and NH₄Cl (17.91 g, 334.90 mmol, 25.00 equiv) under nitrogen atmosphere in a 250 mL round-bottom flask. The mixture was stirred for 17.0 h at 80° C., then the reaction mixture was filtered through a Celite pad. The organic phase was separated, washed with water (50 mL), brine (50 mL), dried with anhydrous Na₂SO₄ and evaporated to give 4.40 g crude of N1-[5-chloro-4-(1H-indol-3-yl)pyrimidin-2-yl]benzene-1,3-diamine as a light yellow solid. LC/MS: mass calcd. For C₁₈H₁₄ClN₅: 335.09, found: 336.10 [M+H]⁺.

### Step 8: Synthesis of tert-butyl N-[(2E)-3-([4-[(3-[[5-chloro-4-(1H-indol-3-yl) pyrimidin-2-yl]amino]phenyl)carbamoyl]phenyl]carbamoyl)prop-2-en-1-yl]-N-methylcarbamate

To a stirred solution of 4-[(2E)-4-[(tert-butoxycarbonyl)(methyl)amino]but-2-enamido]benzoic acid (262.88 mg, 0.79 mmol, 1.10 equiv) in DMF (8.00 mL) was added HATU (326.11 mg, 0.86 mmol, 1.20 equiv), DIEA (277.11 mg, 2.14 mmol, 3.00 equiv) and N1-[5-chloro-4-(1H-indol-3-yl)pyrimidin-2-yl]benzene-1,3-diamine (240.00 mg, 0.72 mmol, 1.00 equiv) at room temperature. The resulting mixture was stirred for 17.0 h at room temperature.

The reaction was quenched with water/ice (20 mL) at 0° C. The precipitated solids were collected by filtration and washed with H₂O (3×5 mL), dried under vacuum. Tert-butyl N-[(2E)-3-([4-[(3-[[5-chloro-4-(1H-indol-3-yl)pyrimidin-2-yl]amino]phenyl)carbamoyl]phenyl]carbamoyl)prop-2-en-1-yl]-N-methylcarbamate (500.00 mg, 85.82% yield) was obtained as a yellow solid. LC/MS: mass calcd. For C₃₅H₃₄ClN₇O₄: 651.24, found: 652.35 [M+H]⁺.

### Step 9: Synthesis of N-(3-[[5-chloro-4-(1H-indol-3-yl)pyrimidin-2-yl] amino]phenyl)-4-[(2E)-4-(methylamino)but-2-enamido]benzamide

To a stirred solution of tert-butyl N-[(2E)-3-([4-[(3-[[5-chloro-4-(1H-indol-3-yl) pyrimidin-2-yl]amino]phenyl)carbamoyl]phenyl]carbamoyl)prop-2-en-1-yl]-N-methylcarbamate (500.00 mg, 0.77 mmol, 1.00 equiv) in DCM (16.00 mL) was added TFA (4.00 mL) at room temperature. The resulting mixture was stirred for 1.0 h at room temperature.

The resulting mixture was concentrated under vacuum. The residue was purified by reverse flash chromatography with the following conditions: column, C18 silica gel; mobile phase, ACN in water (0.05% TFA), 30% to 50% gradient in 15 min; detector, UV 254 nm. The fractions were combined and concentrated. N-(3-[[5-chloro-4-(1H-indol-3-yl)pyrimidin-2-yl]amino]phenyl)-4-[(2E)-4-(methylamino)but-2-enamido]benzamide (330.00 mg, 72.48% yield) was obtained as a yellow oil. LC/MS: mass calcd. For C₃₀H₂₆ClN₇O₂: 551.18, found: 552.30 [M+H]⁺.

### Example 2. Synthesis of N-[3-([4-[1-(benzenesulfonyl)indol-3-yl]-5-chloropyrimidin-2-yl]amino)phenyl]-4-[(2E)-4-bromobut-2-enamido]benzamide (INT-11-Br)

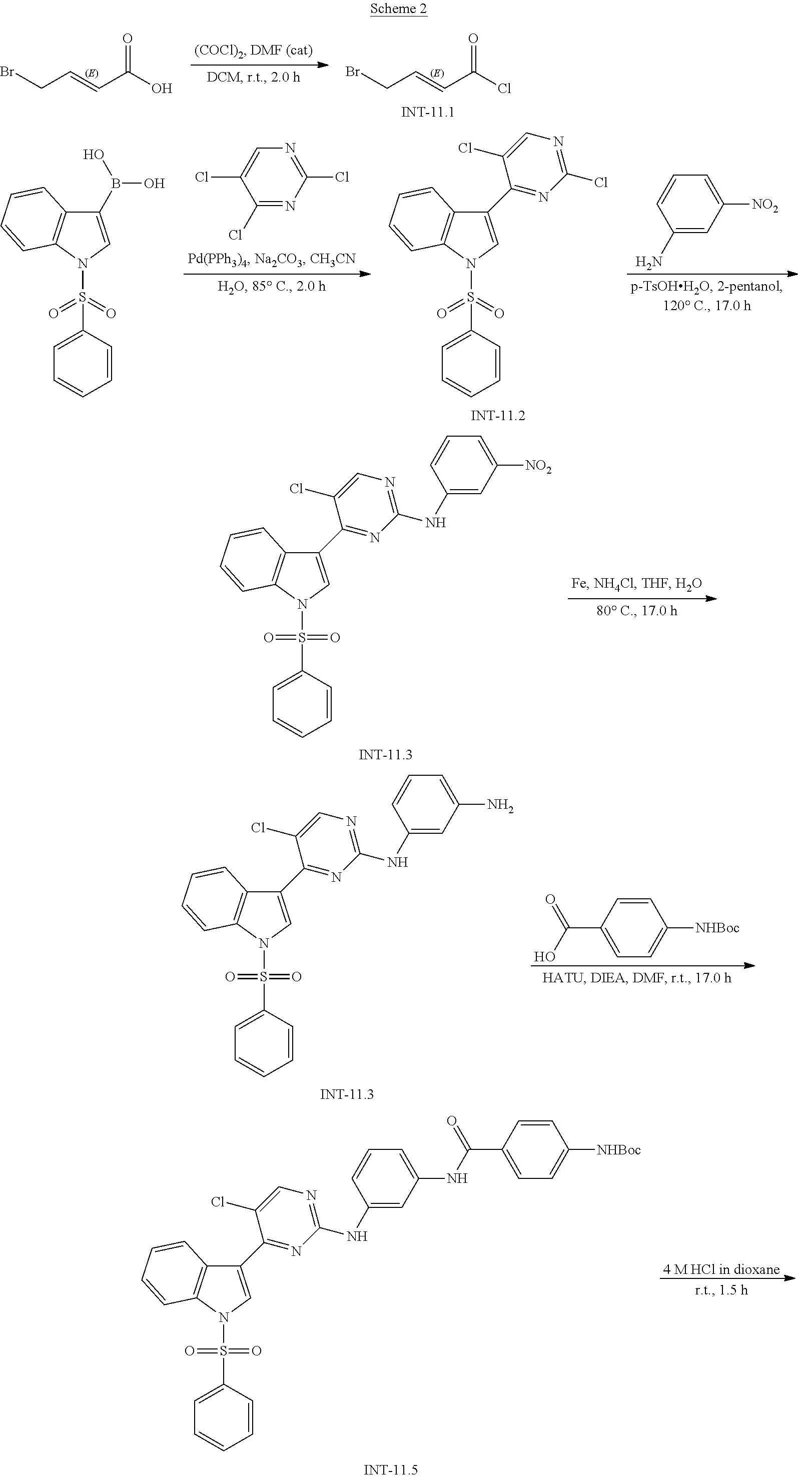

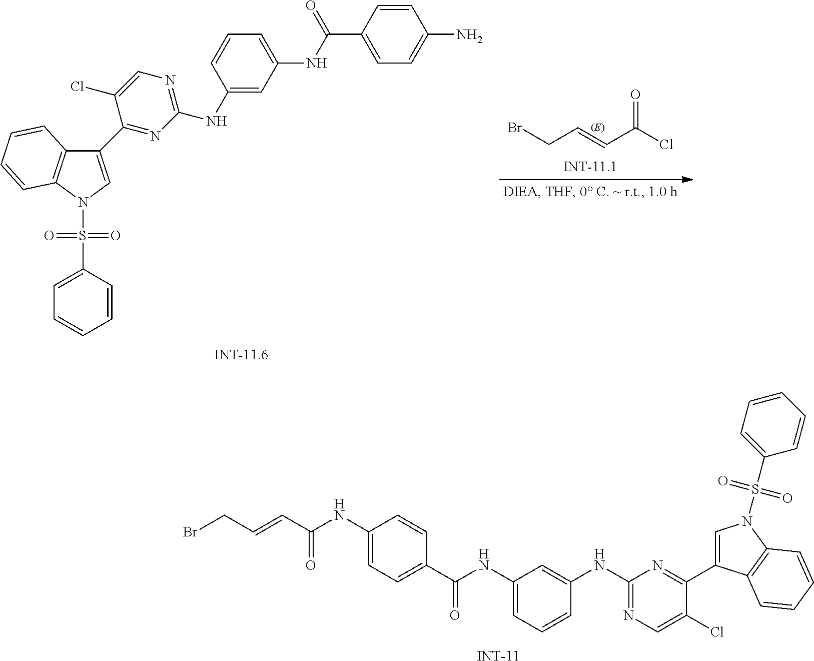

### Step 1: Synthesis of (2E)-4-bromobut-2-enoyl chloride

To a stirred solution of 4-bromo-trans-crotonic acid (1.50 g, 9.09 mmol, 1.00 equiv) in DCM (20.00 mL) was added (COCl)₂ (6.92 g, 54.52 mmol, 6.00 equiv) and DMF (0.50 mL) dropwise at room temperature. The resulting mixture was stirred for 2.0 h at room temperature. The resulting mixture was concentrated under vacuum. (2E)-4-bromobut-2-enoyl chloride (1.50 g, 89.94% yield) was obtained as yellow oil.

### Step 2: Synthesis of 1-(benzenesulfonyl)-3-(2,5-dichloropyrimidin-4-yl)indole

To a stirred mixture of 1(benzenesulfonyl)indolylboronic acid (2.00 g, 6.64 mmol, 1.00 equiv) and 2,4,5-trichloropyrimidine (1.22 g, 6.64 mmol, 1.00 equiv) in acetonitrile (20.00 mL) and H₂O (10.00 mL) were added Na₂CO₃ (1.41 g, 13.28 mmol, 2.00 equiv) and Pd(PPh₃)₄ (0.38 g, 0.33 mmol, 0.05 equiv). N₂ exchanged three times. The reaction mixture was stirred at 85° C. under nitrogen atmosphere for 2.0 h. Then it was filtered, the filter cake was washed with water (3×5 mL), n-hexane (2×5 mL), dried under vacuum. This resulted in 1-(benzenesulfonyl)-3-(2,5-dichloropyrimidin-4-yl)indole (2.60 g, 87.15%) as a white solid. LC/MS: mass calcd. For C₁₈H₁₁Cl₂N₃O₂S: 402.99, found: 403.95 [M+H]⁺.

### Step 3: Synthesis of 4-[1-(Benzenesulfonyl)indol-3-yl]-5-chloro-N-(3-nitrophenyl) pyrimidin-2-amine

To a stirred solution of 1-(benzenesulfonyl)-3-(2,5-dichloropyrimidin-4-yl)indole (2.00 g, 4.95 mmol, 1.00 equiv) and 3-nitroaniline (683.34 mg, 4.95 mmol, 1.00 equiv) in pentan-2-ol (50.00 mL) was added p-toluenesulfonic acid (1.70 g, 9.89 mmol, 2.00 equiv) at room temperature. The resulting mixture was stirred for 17.0 h at 120° C. The resulting mixture was concentrated under reduced pressure. The residue was dissolved in DCM (100 mL) and MeOH (10 mL), and washed with saturated NaHCO₃ (1×50 mL), H₂O (1×50 mL) and brine (1×50 mL), dried over anhydrous Na₂SO₄. After filtration, the filtrate was concentrated under vacuum. 4-[1-(Benzenesulfonyl)indol-3-yl]-5-chloro-N-(3-nitrophenyl)pyrimidin-2-amine (2.20 g, 58.16% yield) was obtained as a yellow solid. LC/MS: mass calcd. For C₂₄H₁₆ClN₅O₄S: 505.06, found: 506.15 [M+H]⁺.

### Step 4: Synthesis of N1-[4-[1-(benzenesulfonyl)indol-3-yl]-5-chloropyrimidin-2-yl]benzene-1,3-diamine

To a stirred solution of 4-[1-(benzenesulfonyl)indol-3-yl]-5-chloro-N-(3-nitrophenyl)pyrimidin-2-amine (2.20 g, 4.35 mmol, 1.00 equiv) in EtOH (30.00 mL) and H₂O (10.00 mL) was added Fe (2.43 g, 43.51 mmol, 10.01 equiv) and NH₄Cl (3.49 g, 65.23 mmol, 15.00 equiv) in portions at room temperature. The resulting mixture was stirred for 17.0 h at 80° C. The mixture was allowed to cool down to room temperature. The resulting mixture was filtered, the filter cake was washed with EA (3×10 mL). The filtrate was concentrated under reduced pressure. The residue was dissolved in H₂O (30 mL), extracted with EA (3×50 mL). The combined organic layers were washed with brine (30 mL), dried over anhydrous Na₂SO₄. After filtration, the filtrate was concentrated under vacuum. Crude N1-[4-[1-(benzenesulfonyl)indol-3-yl]-5-chloropyrimidin-2-yl]benzene-1,3-diamine (2.00 g, 78.48% yield) was obtained as a red oil. LC/MS: mass calcd. For C₂₄H₁₈ClN₅O₂S: 475.09, found: 476.15 [M+H]⁺.

### Step 5: Synthesis of tert-butyl N-(4-[[3-([4-[1-(benzenesulfonyl)indol-3-yl]-S-chloropyrimidin-2-yl]amino)phenyl] carbamoyl]phenyl)carbamate

To a stirred solution of 4-[(tert-butoxycarbonyl)amino]benzoic acid (1.00 g, 4.20 mmol, 1.00 equiv) in DMF (25.00 mL) was added HATU (2.40 g, 6.30 mmol, 1.50 equiv), DIEA (1.63 g, 12.61 mmol, 3.00 equiv) and N1-[4-[1-(benzenesulfonyl)indol-3-yl]-5-chloropyrimidin-2-yl]benzene-1,3-diamine (2.00 g, 4.20 mmol, 1.00 equiv) at 0° C. The resulting mixture was stirred for 17.0 h at room temperature. The reaction was quenched with Water/Ice (75 mL) at 0° C. The resulting mixture was extracted by EA (3×100 mL). The combined organic layers were washed with brine (3×100 mL), dried over anhydrous Na₂SO₄. After filtration, the filtrate was concentrated under reduced pressure.

The residue was purified by silica gel column, eluted with PE/EA (3:2) to afford tert-butyl N-(4-[[3-([4-[1-(benzenesulfonyl)indol-3-yl]-5-chloropyrimidin-2-yl]amino)phenyl]carbamoyl]phenyl)carbamate (2.00 g, 61.16% yield) as a yellow solid. LC/MS: mass calcd. For C₃₆H₃₁ClN₆O₅S: 694.18, found: 695.10 [M+H]⁺.

### Step 6: Synthesis of 4-Amino-N-[3-([4-[1-(benzenesulfonyl)indol-3-yl]-5-chloropyrimidin-2-yl]amino)phenyl]benzamide

A solution of tert-butyl N-(4-[[3-([4-[1-(benzenesulfonyl)indol-3-yl]-5-chloropyrimidin-2-yl]amino)phenyl]carbamoyl]phenyl)carbamate (2.00 g, 2.88 mmol, 1.00 equiv) in HCl in 1,4-dioxane (4M, 20.00 mL) was stirred for 1.5 h at room temperature. The resulting mixture was concentrated under vacuum. 4-Amino-N-[3-([4-[1-(benzenesulfonyl)indol-3-yl]-5-chloropyrimidin-2-yl]amino)phenyl]benzamide (2.00 g, crude) was obtained as a yellow solid. LC/MS: mass calcd. For C₃₁H₂₃ClN₆O₃S: 594.12, found: 595.25 [M+H]⁺.

### Step 7: Synthesis of N-[3-([4-[1-(benzenesulfonyl)indol-3-yl]-5-chloropyrimidin-2-yl]amino)phenyl]-4-[(2E)-4-bromobut-2-enamido]benzamide

To a stirred solution of 4-amino-N-[3-([4-[1-(benzenesulfonyl)indol-3-yl]-5-chloropyrimidin-2-yl] amino)phenyl]benzamide (1.90 g, 3.19 mmol, 1.00 equiv) in THF (20.00 mL) was added DIEA (1.24 g, 9.58 mmol, 3.00 equiv) and the solution of (2E)-4-bromobut-2-enoyl chloride (0.59 g, 3.22 mmol, 1.01 equiv) in THF (10.00 mL) dropwise at 0° C. The resulting mixture was stirred for 1.0 h at room temperature.

The resulting mixture was concentrated under vacuum. The residue was purified by silica gel column chromatography, eluted with PE/EA (1:4) to afford N-[3-[4-[1-(benzenesulfonyl)indol-3-yl]-5-chloropyrimidin-2-yl]amino)phenyl]-4-[(2E)-4-bromobut-2-enamido]benzamide (1.60 g, 64.21% yield) as a yellow solid. LC/MS: mass calcd. For C₃₅H₂₆BrClN₆O₄S: 740.06, 742.06, found: 741.00, 743.00 [M+H, M+2+H]⁺.

### Example 3. Synthesis of (2E)-N-[4-[(3R)-3-[[5-chloro-4-(1H-indol-3-yl)pyrimidin-2-yl]amino]piperidine-1-carbonyl]phenyl]-4-(methylamino)but-2-enamide (INT-12)

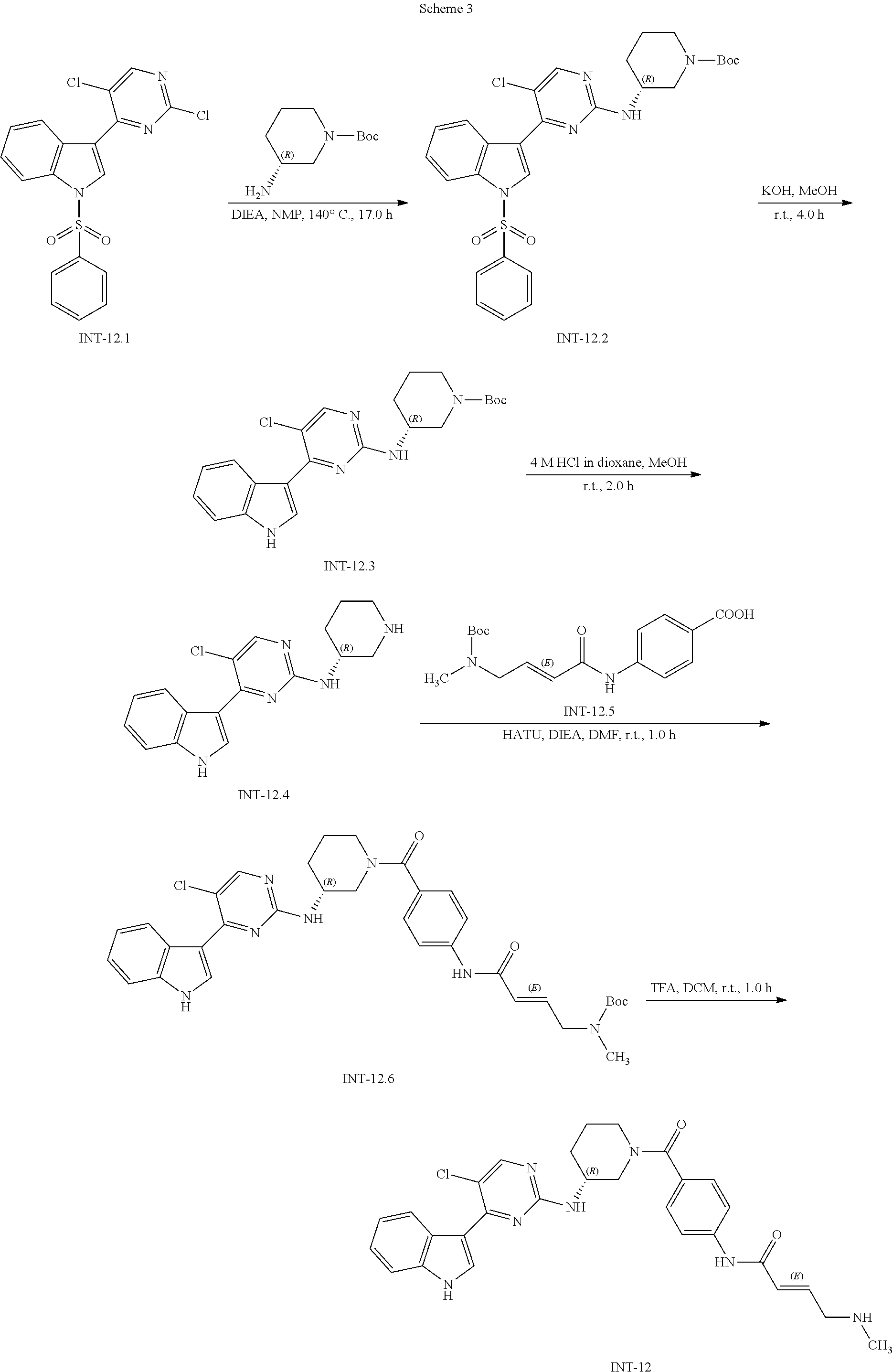

### Step 1: Synthesis of tert-butyl (3R)-3-([4-[1-(benzenesulfonyl)indol-3-yl]-5-chloropyrimidin-2-yl]amino)piperidine-1-carboxylate

To a stirred mixture of 1-(benzenesulfonyl)-3-(2,5-dichloropyrimidin-4-yl)indole (4.00 g, 9.89 mmol, 1.00 equiv) and tert-butyl (3R)-3-aminopiperidine-1-carboxylate (3.21 g, 16.03 mmol, 1.62 equiv) in NMP (50.00 mL) was added DIEA (1.91 g, 14.84 mmol, 15.00 equiv). The reaction mixture was stirred at 140° C. for 17.0 h. The reaction was quenched with water (200 mL) at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (1:1) to afford tert-butyl (3R)-3-([4-[1-(benzenesulfonyl)indol-3-yl]-5-chloropyrimidin-2-yl]amino)piperidine-1-carboxylate (5.00 g, 66.63% yield) as a brown oil. LC/MS: mass calcd. For C₂₈H₃₀ClN₅O₄S: 567.17, found: 568.20 [M+H]⁺.

### Step 2: Synthesis of tert-butyl (3R)-3-[[5-chloro-4-(1H-indol-3-yl) pyrimidin-2-yl]amino]piperidine-1-carboxylate

To a stirred solution of tert-butyl (3R)-3-([4-[1-(benzenesulfonyl)indol-3-yl]-5-chloropyrimidin-2-yl] amino)piperidine-1-carboxylate (4.00 g, 7.04 mmol, 1.00 equiv) in MeOH (50.00 mL) was added KOH (1.58 g, 28.16 mmol, 4.00 equiv) in H₂O (25.00 mL) at room temperature. The resulting mixture was stirred for 4.0 h at room temperature.

The resulting mixture was concentrated under vacuum. The residue was purified by silica gel column chromatography, eluted with PE/EA (2:1) to afford tert-butyl (3R)-3-[[5-chloro-4-(1H-indol-3-yl)pyrimidin-2-yl]amino]piperidine-1-carboxylate (2.00 g, 63.53% yield) as a yellow solid. LC/MS: mass calcd. For C₂₂H₂₆ClN₅O₂: 427.18, found: 428.10 [M+H]⁺.

### Step 3: Synthesis of 5-Chloro-4-(1H-indol-3-yl)-N-[(3R)-piperidin-3-yl] pyrimidin-2-amine

To a stirred solution of tert-butyl (3R)-3-[[5-chloro-4-(1H-indol-3-yl)pyrimidin-2-yl]amino]piperidine-1-carboxylate (500.00 mg, 1.17 mmol, 1.00 equiv) in MeOH (5.00 mL) was added HCl (gas) in 1,4-dioxane (4M, 15.00 mL) at room temperature. The resulting mixture was stirred for 2.0 h at room temperature. The resulting mixture was concentrated under vacuum. 5-Chloro-4-(1H-indol-3-yl)-N-[(3R)-piperidin-3-yl] pyrimidin-2-amine (500.00 mg, crude) was obtained as a yellow solid. LC/MS: mass calcd. For C₁₇H₁₈ClN₅: 327.13, found: 328.15 [M+H]⁺.

### Step 4: Synthesis of tert-butyl N-[(2E)-3-([4-[(3R)-3-[[5-chloro-4-(1H-indol-3-yl) pyrimidin-2-yl]amino]piperidine-1-carbonyl]phenyl]carbamoyl)prop-2-en-1-yl]-N-methylcarbamate

The procedure was the same as tert-butyl N-[(2E)-3-([4-[(3-[[5-chloro-4-(1H-indol-3-yl)pyrimidin-2-yl]amino]phenyl)carbamoyl]phenyl]carbamoyl)prop-2-en-1-yl]-N-methylcarbamate (INT-10.8). 500.00 mg of 5-chloro-4-(1H-indol-3-yl)-N-[(3R)-piperidin-3-yl]pyrimidin-2-amine was used, 900.00 mg of desired product was obtained as yellow solid (69.14% yield). LC/MS: mass calcd. For C₃₄H₃₈ClN₇O₄: 643.27, found: 644.35 [M+H]⁺.

### Step 5: Synthesis of (2E)-N-[4-[(3R)-3-[[5-chloro-4-(1H-indol-3-yl) pyrimidin-2-yl]amino]piperidine-1-carbonyl]phenyl]-4-(methylamino)but-2-enamide

The procedure was the same as N-(3-[[5-chloro-4-(1H-indol-3-yl)pyrimidin-2-yl]amino]phenyl)-4-[(2E)-4-(methylamino)but-2-enamido]benzamide (INT-10). 900.00 mg of tert-butyl N-[(2E)-3-([4-[(3R)-3-[[5-chloro-4-(1H-indol-3-yl) pyrimidin-2-yl]amino]piperidine-1-carbonyl]phenyl]carbamoyl)prop-2-en-1-yl]-N-methylcarbamate was used, 450.00 mg of desired product was obtained as light yellow solid (59.20% yield). LC/MS: mass calcd. For C₂₉H₃₀ClN₇O₂: 543.21, found: 544.15 [M+H]⁺.

### Example 4. Synthesis of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoic acid (INT-14)

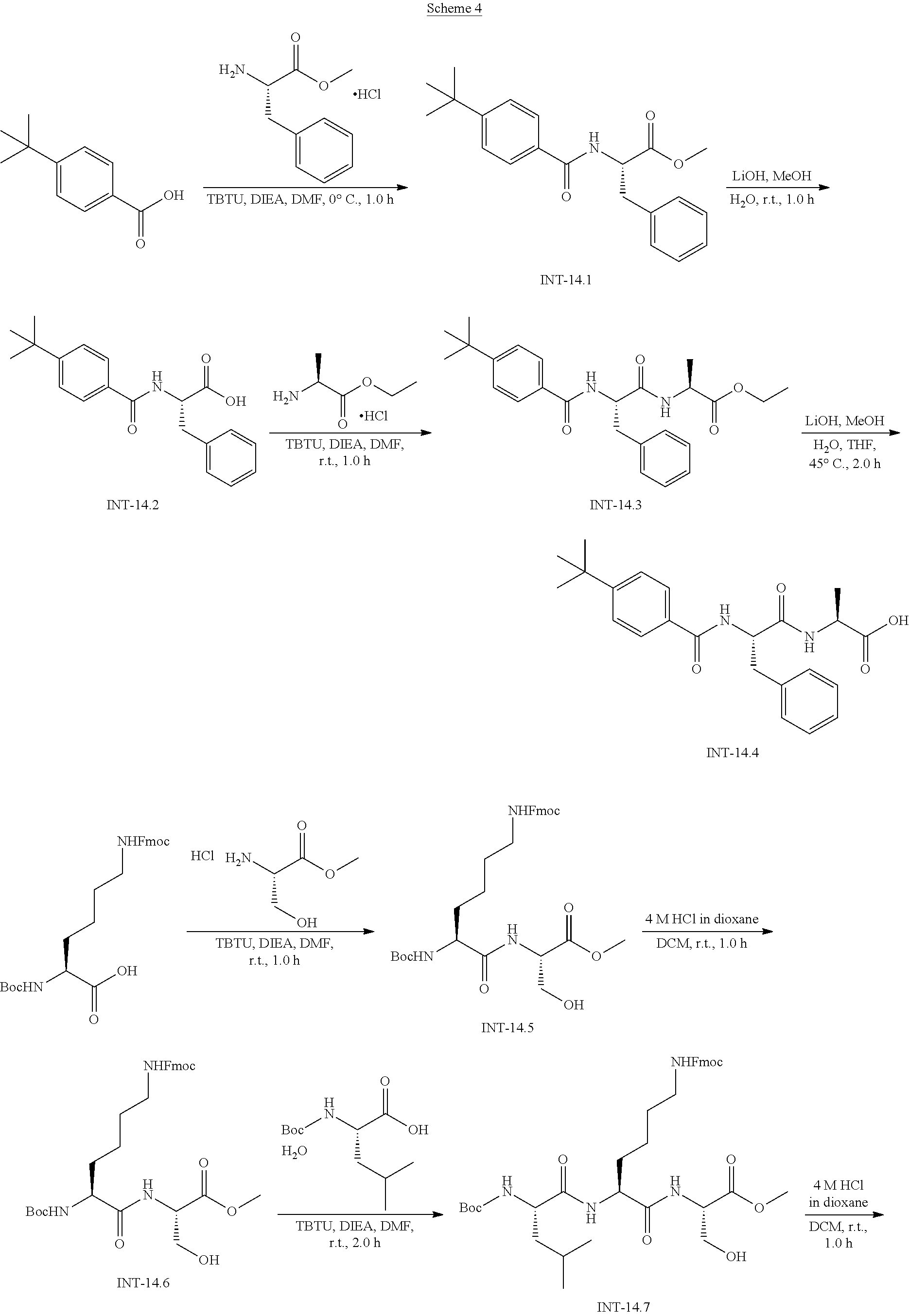

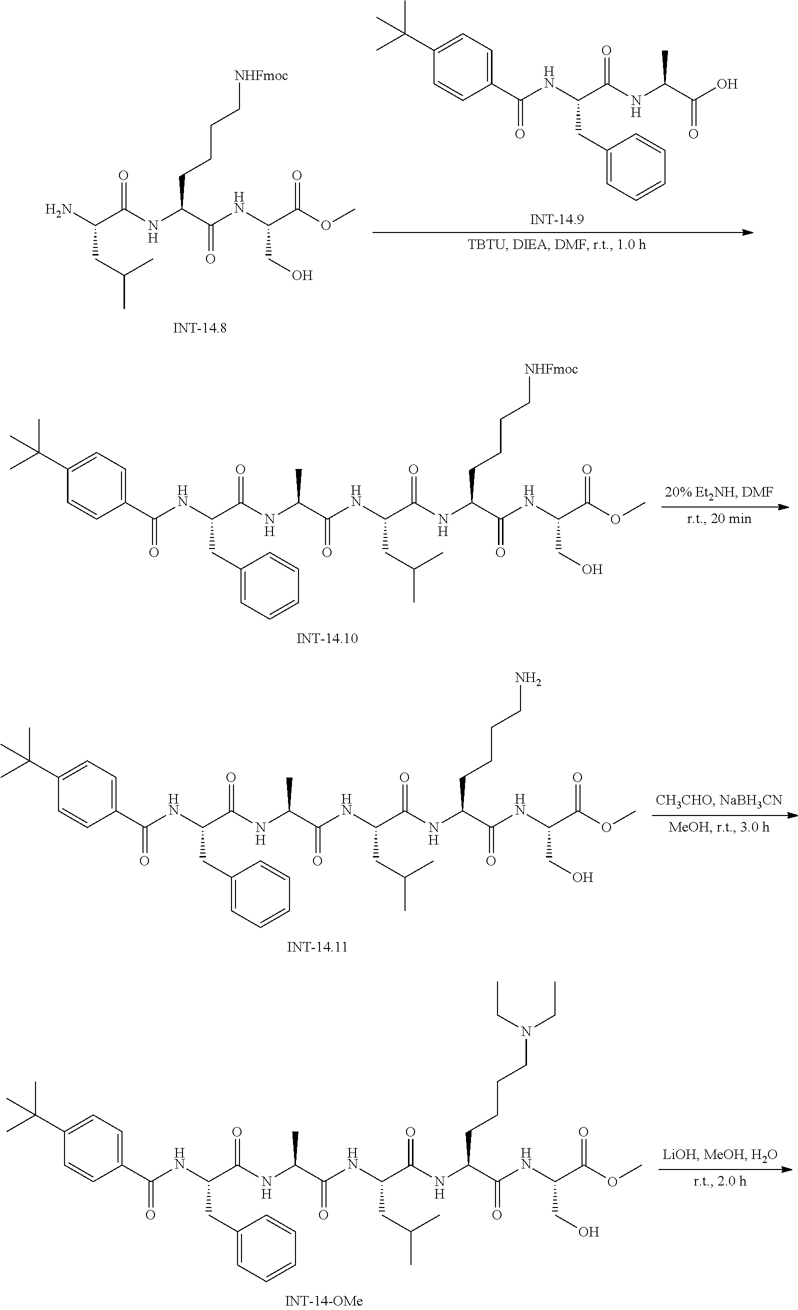

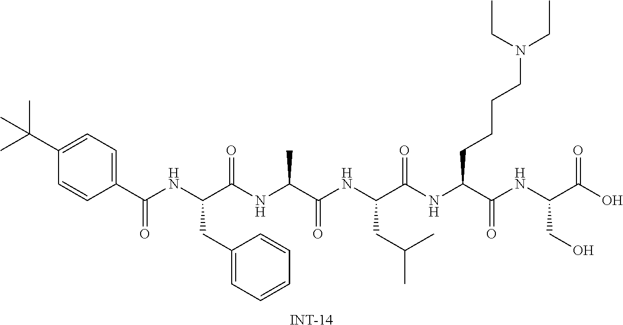

### Step 1: Synthesis of methyl (2S)-2-[(4-tert-butylphenyl)formamido]-3-phenylpropanoate

Into a 1000 ml flask was added 4-tert-butylbenzoic acid (10.00 g, 56.11 mmol, 1.00 equiv) and DMF (250.00 mL), the solution was stirred at 0° C. for 10 mins, methyl L-phenylalaninate (12.07 g, 67.33 mmol, 1.20 equiv), TBTU (23.42 g, 72.94 mmol, 1.30 equiv), DIEA (18.13 g, 140.27 mmol, 2.50 equiv) were added, the reaction was stirred at 0° C. for 1.0 h. The reaction was poured into ice water (800 mL), the precipitated solids were collected by filtration and washed with water (3×100 mL), dried under vacuum. This resulted in methyl (2S)-2-[(4-tert-butylphenyl)formamido]-3-phenylpropanoate (20.00 g, crude) as white solid. LC/MS: mass calcd. For C₂₁H₂₅NO₃: 339.18, found: 340.20[M+H]⁺.

### Step 2: Synthesis of (2S)-2-[(4-tert-butylphenyl)formamido]-3-phenylpropanoic acid

Into a 1000 ml flask was added methyl (2S)-2-[(4-tert-butylphenyl)formamido]-3-phenylpropanoate (19.00 g, 55.98 mmol, 1.00 equiv), MeOH (160.00 mL), LiOH·H₂O (2M, 83.96 mL, 167.93 mmol, 3.00 equiv), THF (80.00 mL), the reaction was stirred at room temperature for 1.0 h. The reaction was concentrated, the residue was dissolved in water (200 mL), cooled to 0° C., adjust to pH=5 by 2M hydrochloric acid. The precipitated solids were collected by filtration and washed with water (3×50 mL), dried under vacuum. This resulted in (2S)-2-[(4-tert-butylphenyl)formamido]-3-phenylpropanoic acid (19.00 g, crude) as a white solid. LCMS: mass calcd. For C₂₀H₂₃NO₃: 325.17, found: 326.15[M+H]⁺.

### Step 3: Synthesis of methyl (2S)-2-[(2S)-2-[(4-tert-butylphenyl)formamido]-3-phenylpropanamido]propanoate

The procedure was the same as methyl (2S)-2-[(4-tert-butylphenyl)formamido]-3-phenylpropanoate (INT14.1), but the reaction temperature was r.t. 10.00 g of (2S)-2-[(4-tert-butylphenyl) formamido]-3-phenylpropanoic acid was used, 13.00 g crude of methyl (2S)-2-[(2S)-2-[(4-tert-butylphenyl)formamido]-3-phenylpropanamido]propanoate was obtained as off-white solid. LC/MS: mass calcd. For C₂₅H₃₂N₂O₄: 424.24, found: 425.25[M+H]⁺.

### Step 4: Synthesis of (2S)-2-[(2S)-2-[(4-tert-butylphenyl)formamido]-3-phenylpropanamido]propanoic acid

The procedure was the same as methyl (2S)-2-[(4-tert-butylphenyl)formamido]-3-phenylpropanoate (INT-14.2). 13.00 g of methyl (2S)-2-[(2S)-2-[(4-tert-butylphenyl)formamido]-3-phenylpropanamido]propanoate was used, 12.00 g of (2S)-2-[(2S)-2-[(4-tert-butylphenyl)formamido]-3-phenylpropanamido]propanoic acid was obtained as off-white solid (95.68% yield). LC/MS: mass calcd. For C₂₃H₂₈N₂O₄: 396.20, found: 397.25[M+H]⁺.

### Step 5: Synthesis of methyl (2S)-2-[(2S)-2-[(tert-butoxycarbonyl)amino]-6-{[(9H-fluoren-9-ylmethoxy)carbonyl]amino}hexanamido]-3-hydroxypropanoate

The procedure was the same as methyl (2S)-2-[(4-tert-butylphenyl)formamido]-3-phenylpropanoate (INT-14.1), but the reaction temperature was r.t. 10.00 g of (2S)-2-[(tert-butoxycarbonyl)amino]-6-{[(9H-fluoren-9-ylmethoxy)carbonyl] amino}hexanoic acid was used, _{12.00} g crude of desired product was obtained as white solid (94.99% yield). LC/MS: mass calcd. For C₃₀H₃₉N₃O₈: 569.27, found: 592.45[M+Na]⁺.

### Step 6: Synthesis of methyl (2S)-2-[(2S)-2-amino-6-{[(9H-fluoren-9-yl)methoxy) carbonyl]amino}hexanamido]-3-hydroxypropanoate

A solution of methyl (2S)-2-[(2S)-2-[(tert-butoxycarbonyl)amino]-6-{[(9H-fluoren-9-ylmethoxy)carbonyl]amino}hexanamido]-3-hydroxypropanoate (13.00 g, 22.82 mmol, 1.00 equiv) and HCl (gas) in 1,4-dioxane (50.00 mL, 4M) in DCM (100.00 mL) was stirred for 1.0 h at room temperature. The resulting mixture was concentrated under vacuum. The resulting solid was washed with Et₂O (2×50 mL) and dried under vacuum. This resulted in methyl (2S)-2-[(2S)-2-amino-6-{[(9H-fluoren-9-ylmethoxy)carbonyl]amino}hexanamido]-3-hydroxypropanoate (12.00 g, crude) as a white solid. LC/MS: mass calcd. For C₂₅H₃₁N₃O₆: 469.22, found: 470.40[M+H]⁺.

### Step 7: Synthesis of methyl (2S)-2-[(2S)-2-[(2S)-2-[(tert-butoxycarbonyl)amino]-4-methylpentanamido]-6-{[(9H-fluoren-9-ylmethoxy)carbonyl]amino}hexanamido]-3-hydroxypropanoate

The procedure was the same as methyl (2S)-2-[(4-tert-butylphenyl)formamido]-3-phenylpropanoate (INT-14.1), but the reaction temperature was r.t. and the reaction time was 2.0 h. 9.00 g of methyl (2S)-2-[(2S)-2-amino-6-{[(9H-fluoren-9-ylmethoxy)carbonyl]amino}hexanamido]-3-hydroxypropanoate was used, 8.50 g of desired product was obtained as white solid (64.99% yield). LC/MS: mass calcd. For C₃₆H₅₀N₄O₉: 682.36, found: 683.55[M+H]⁺.

### Step 8: Synthesis of methyl (2S)-2-[(2S)-2-[(2S)-2-amino-4-methylpentanamido]-6-{[(9H-fluoren-9-ylmethoxy)carbonyl]amino}hexanamido]-3-hydroxypropanoate

The procedure was the same as methyl (2S)-2-[(2S)-2-amino-6-{[(9H-fluoren-9-yl)methoxy)carbonyl]amino}hexanamido]-3-hydroxypropanoate (INT-14.6). 8.50 g of methyl (2S)-2-[(2S)-2-[(2S)-2-[(tert-butoxycarbonyl)amino]-4-methylpentanamido]-6-{[(9H-fluoren-9-ylmethoxy)carbonyl]amino}hexanamido]-3-hydroxypropanoate was used, 8.40 g crude of desired product was obtained as white solid. LC/MS: mass calcd. For C₃₁H₄₂N₄O₇: 582.31, found: 583.50[M+H]⁺.

### Step 9: Synthesis of methyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-{[(9H-fluoren-9-ylmethoxy)carbonyl]amino}hexanamido]-3-hydroxypropanoate

The procedure was the same as methyl (2S)-2-[(4-tert-butylphenyl) formamido]-3-phenylpropanoate (INT-14.1), but the reaction temperature was r.t. And the obtained solid was purified by PREP_ACHIRAL_SFC, Column: GreenSep Basic, 3*15 cm, 5 μm; Mobile Phase A: CO2, Mobile Phase B: MeOH (0.5% 2M NH3-MeOH)-HPLC; Flow rate: 75 mL/min; Gradient: isocratic 30% B; Column Temperature (° C.): 35; Back Pressure (bar): 100; Wave Length: 254 nm; RT1 (min): 4.82; Sample Solvent: MeOH-HPLC. 8.00 g of methyl (2S)-2-[(2S)-2-[(2S)-2-amino-4-methylpentanamido]-6-{[(9H-fluoren-9-ylmethoxy)carbonyl]amino}hexanamido]-3-hydroxypropanoate was used, 4.80 g of desired product was obtained as yellow solid (40.23% yield). LC/MS: mass calcd. For C₅₄H₆₈N₆O₁₀: 960.50, found: 961.95[M+H]⁺.

### Step 10: Synthesis of methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate

A solution of methyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-{[(9H-fluoren-9-ylmethoxy)carbonyl]amino}hexanamido]-3-hydroxypropanoate (2.60 g, 2.71 mmol, 1.00 equiv) and diethylamine (2.00 mL) in DMF (8.00 mL) was stirred for 20 min at room temperature. The resulting solution was purified by reverse flash chromatography with the following conditions: column, C18 silica gel; mobile phase, ACN in water (0.05% TFA), 5% to 38% gradient in 30 min; detector, UV 254 nm. The fractions was combined and evaporated to afford the methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (2.00 g, 85.05%) as a yellow solid. LC/MS: mass calcd. For C₃₉H₅₈N₆O₈: 738.43, found: 739.65[M+H]⁺.

### Step 11: methyl (2S)-2-[(2S)-2-[(2S)-2-[(2S)-2-[(2S)-2-[(4-tert-butylphenyl) formamido]-3-phenylpropanamido]propanamido]-4-methylpentanamido]-6-(diethylamino)hexanamido]-3-hydroxypropanoate

To a stirred solution of methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (1.80 g, 2.44 mmol, 1.00 equiv) and acetaldehyde (428.00 mg, 9.72 mmol, 3.99 equiv) in MeOH (10.00 mL) was added NaBH₃CN (459.00 mg, 7.30 mmol, 3.00 equiv) in portions at room temperature. The resulting mixture was stirred for 3.0 h at room temperature. The resulting mixture was concentrated under reduced pressure. The resulting mixture was diluted with water (150 mL). The resulting mixture was extracted with CH₂Cl₂ (3×150 mL). The combined organic layers were washed with brine (2×50 mL), dried over anhydrous Na₂SO₄. After filtration, the filtrate was concentrated under reduced pressure. The residue was purified by silica gel column chromatography, eluted with CH₂Cl₂/MeOH (10:1) to afford methyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoate (1.75 g, 90.50% yield) as a yellow oil. HRMS: mass calcd. For C₄₃H₆₆N₆O₈: 794.4942, found: 795.5154 [M+H]⁺.

### Step 12: Synthesis of (2S)-2-[(2S)-2-[(2S)-2-[(2S)-2-[(2S)-2-[(4-tert-butylphenyl) formamido]-3-phenylpropanamido]propanamido]-4-methylpentanamido]-6-(diethylamino)hexanamido]-3-hydroxypropanoic acid

The procedure was the same as (2S)-2-[(4-tert-butylphenyl)formamido]-3-phenylpropanoic acid (INT-14.2), but the solvent was MeOH/H₂O, reaction temperature was 30° C. and reaction time was 2.0 h. 1.60 g of methyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoate was used, 1.70 g crude of desired product was obtained as white solid. LC/MS: mass calcd. For C₄₂H₆₄N₆O₈: 780.48, found: 781.45[M+H]⁺.

### Example 5. Synthesis of 4-([2,5-bis[4-(pyrrolidin-1-yl)piperidine-1-carbonyl] phenyl]amino)benzoic acid (INT-18)

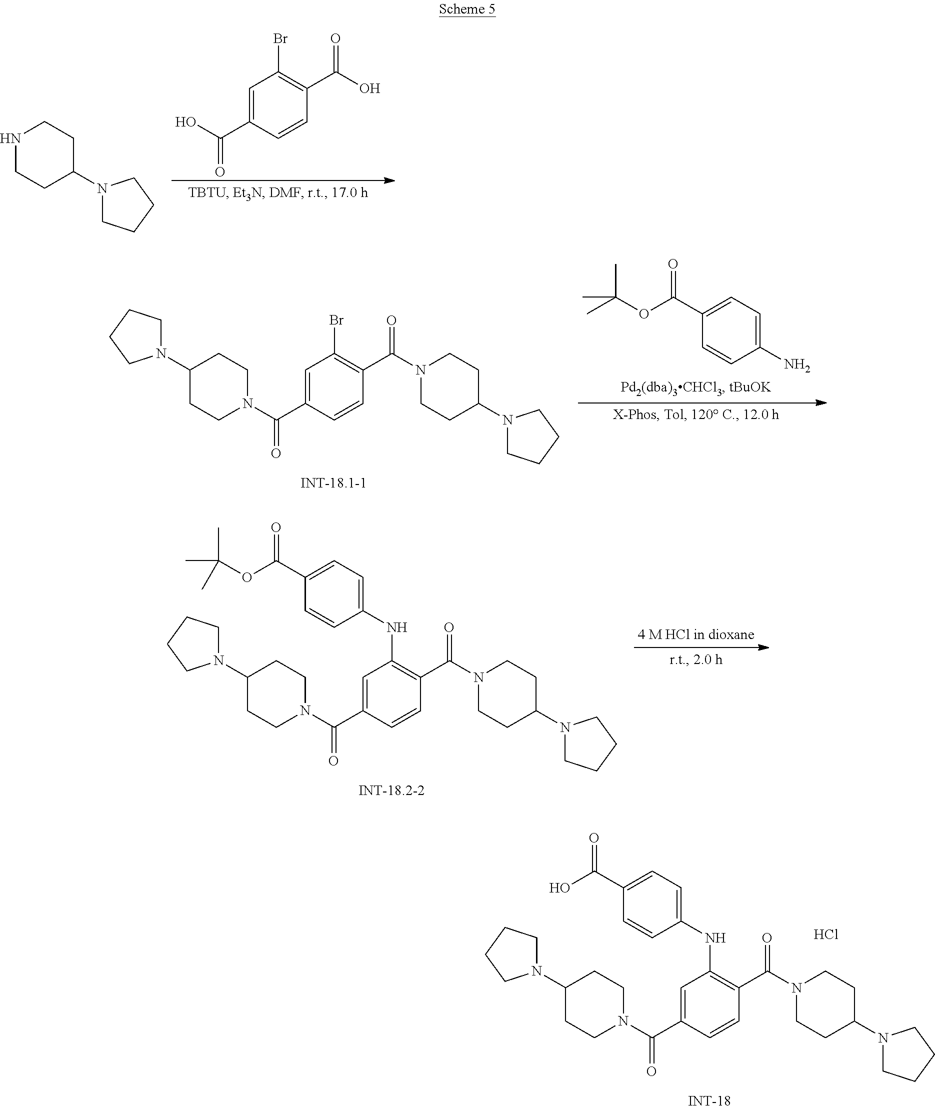

### Step 1: Synthesis of 1-[3-bromo-4-[4-(pyrrolidin-1-yl)piperidine-1-carbonyl] benzoyl]-4-(pyrrolidin-1-yl) piperidine

To a solution of 2-bromobenzene-1,4-dicarboxylic acid (1.00 g, 4.08 mmol, 1.00 equiv) in DMF (25.00 mL) was added TBTU (3.12 g, 12.24 mmol, 3.00 equiv) and triethylamine (1.23 g, 12.20 mmol, 2.99 equiv). Then 4-(pyrrolidin-1-yl)piperidine (1.26 g, 8.16 mmol, 2.00 equiv) was added. The mixture was stirred at r.t. overnight. The mixture was poured into 50 mL ice water, EA extracted (3×50 mL). The organic phases were combined and washed by H₂O (2×50 mL) and brine (50 mL), dried over anhydrous Na₂SO₄. The solid was filtered out and concentrated. The residue was purified by silica gel column with DCM/MeOH=5:1. 1.00 g desired product was obtained as brown solid (46.00% yield). LC/MS: mass calcd. For C₂₆H₃₇BrN₄O₂: 516.21, found: 517.05, 519.05 [M+H, M+2+H]⁺.

### Step 2: Synthesis of tert-butyl 4-([2,5-bis[4-(pyrrolidin-1-yl)piperidine-1-carbonyl]phenyl]amino)benzoate

To a solution of 1-[3-bromo-4-[4-(pyrrolidin-1-yl)piperidine-1-carbonyl]benzoyl]-4-(pyrrolidin-1-yl)piperidine (1.00 g, 1.93 mmol, 1.00 equiv) in Tol (25.00 mL) was added tert-butyl 4-aminobenzoate (373.41 mg, 1.93 mmol, 1.00 equiv), potassium tert-butoxide (433.66 mg, 3.865 mmol, 2.0 equiv), X-Phos (184.23 mg, 0.39 mmol, 0.20 equiv) and Pd₂(dba)₃·CHCl₃ (200.01 mg, 0.19 mmol, 0.10 equiv). N₂ exchanged three times. The mixture was stirred for 12.0 h at 120° C. The solvent was removed and the residue was purified by silica gel column with MeOH/DCM=1:5. 950.00 mg desired product was obtained as brown solid (58.00% yield). LC/MS: mass calcd. For C₃₇H₅₁N₅O₄: 629.39, found: 630.50 [M+H]⁺.

### Step 3: Synthesis of 4-([2,5-bis[4-(pyrrolidin-1-yl)piperidine-1-carbonyl]phenyl]amino)benzoic acid

Into a 100 mL flask was added tert-butyl 4-([2,5-bis[4-(pyrrolidin-1-yl) piperidine-1-carbonyl]phenyl]amino)benzoate (950.00 mg, 1.51 mmol, 1.00 equiv) and 4M HCl in dioxane solution (20.00 mL). The mixture was stirred at r.t. for 2.0 h. The solvent was removed and the residue was washed with Et₂O (2×10 mL), dried under vacuum. This resulted 850 mg desired product as light brown solid (90.00% yield). LC/MS: mass calcd. For C₃₃H₄₃N₅O₄: 573.33, found: 574.50 [M+H]⁺.

### Example 6. Synthesis of tert-butyl 3-[[4-([[4-hydroxy-2-(pyrazol-1-yl)phenyl]methyl]amino)-8-isopropylpyrazolo[1,5-a][1,3,5]triazin-2-yl]oxy]piperidine-1-carboxylate (INT-22-Boc)

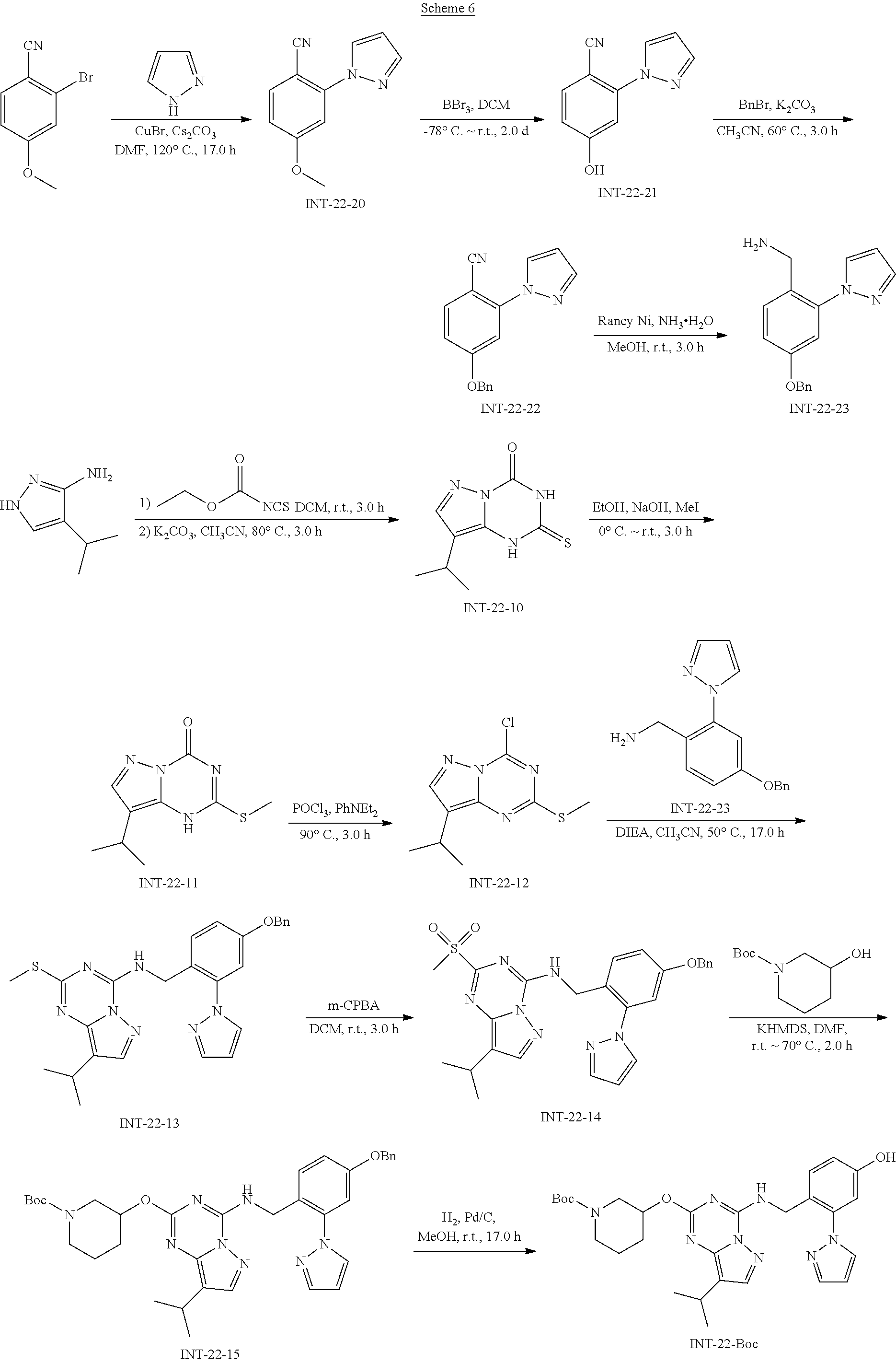

### Step 1: Synthesis of 4-methoxy-2-(pyrazol-1-yl)benzonitrile

To a stirred solution of 2-bromo-4-methoxybenzonitrile (4.50 g, 21.22 mmol, 1.00 equiv) in DMF (80.00 mL) was added pyrazole (7.22 g, 106.05 mmol, 5.00 equiv), Cs₂CO₃ (13.83 g, 42.44 mmol, 2.00 equiv) and CuBr (6.09 g, 42.44 mmol, 2.00 equiv) in portions at room temperature. The resulting mixture was stirred for 17.0 h at 120° C. under N₂ atmosphere. The resulting mixture was filtered and the filtrated was diluted with H₂O (150 mL). The resulting mixture was extracted with EA (3×200 mL). The combined organic layers were washed with brine (3×200 mL), dried over anhydrous Na₂SO₄. After filtration, the filtrate was concentrated under reduced pressure. The residue was purified by silica gel column, eluted with PA/EA (3:1) to afford 4-methoxy-2-(pyrazol-1-yl)benzonitrile (1.70 g, 39.44% yield) as a white solid. LC/MS: mass calcd. For C₁₁H₉N₃O: 199.07, found: 200.05 [M+H]⁺.

### Step 2: Synthesis of 4-Hydroxy-2-(pyrazol-1-yl)benzonitrile

To a stirred solution of 4-methoxy-2-(pyrazol-1-yl)benzonitrile (1.30 g, 6.53 mmol, 1.00 equiv) in DCM (20.00 mL) was added boron tribromide (32.70 g, 0.13 mmol, 20.00 equiv) dropwise at −78° C. The resulting mixture was stirred for two days at room temperature.

The reaction was quenched with water/ice (20 mL) at 0° C.
The resulting mixture was extracted with DCM (3×50 mL). The combined organic layers were washed with brine (1×50 mL), dried over anhydrous Na₂SO₄. After filtration, the filtrate was concentrated under vacuum. 4-Hydroxy-2-(pyrazol-1-yl)benzonitrile (1.20 g, 84.72% yield) was obtained as a yellow solid. LC/MS: mass calcd. For C₁₀H₇N₃O: 185.06, found: 186.10 [M+H]⁺.

### Step 3: Synthesis of 4-(benzyloxy)-2-(pyrazol-1-yl)benzonitrile

To a stirred solution of 4-hydroxy-2-(pyrazol-1-yl)benzonitrile (1.10 g, 5.94 mmol, 1.00 equiv) in CH₃CN (20.00 mL) was added BnBr (1.52 g, 5.89 mmol, 1.50 equiv) and K₂CO₃ (2.46 g, 17.82 mmol, 3.00 equiv) in portions at room temperature. The resulting mixture was stirred for 3.0 h at 60° C. The solid was filtered out and filtrate was concentrated under vacuum. The residue was purified by silica gel column chromatography, eluted with PE/EA (4:1) to afford 4-(benzyloxy)-2-(pyrazol-1-yl)benzonitrile (1.20 g, 73.07% yield) as a white solid. LC/MS: mass calcd. For C₁₇H₁₃N₃O: 275.11, found: 276.10 [M+H]⁺.

### Step 4: Synthesis of 1-[4-(Benzyloxy)-2-(pyrazol-1-yl)phenyl]methanamine

To a stirred solution of 4-(benzyloxy)-2-(pyrazol-1-yl)benzonitrile (1.20 g, 4.36 mmol, 1.00 equiv) in MeOH (20.00 mL) was added Raney Ni (373.43 mg, 4.36 mmol, 1.00 equiv) and NH₃·H₂O (76.38 mg, 2.18 mmol, 0.50 equiv) in portions at room temperature. Then H₂ was exchanged by three times. The resulting mixture was stirred for 3.0 h at room temperature under H₂ atmosphere. The solid was filtered out and the filter cake was washed with MeOH (3×10 mL). The filtrate was concentrated under vacuum. 1-[4-(Benzyloxy)-2-(pyrazol-1-yl)phenyl]methanamine (1.20 g, 86.69% yield) was obtained as a yellow oil. LC/MS: mass calcd. For C₁₇H₁₇N₃O: 279.14, found: 280.15 [M+H]⁺.

### Step 5: Synthesis of 8-Isopropyl-2-sulfanylidene-1H,3H-pyrazolo[1,5-a][1,3,5]triazin-4-one

To a stirred solution of 4-isopropyl-1H-pyrazol-3-amine (2.50 g, 19.97 mmol, 1.00 equiv) in DCM (40.00 mL) was added ethyl N-carbothioylcarbamate (2.62 g, 19.98 mmol, 1.00 equiv) in DCM (20.00 mL) dropwise at room temperature. The resulting mixture was stirred for 3.0 h at room temperature. The precipitated solids were collected by filtration and washed with DCM (3×10 mL), dried under vacuum.

The residue was dissolved in CH₃CN (45.00 mL). To the above mixture was added K₂CO₃ (8.28 g, 59.92 mmol, 3.00 equiv) in portions at room temperature. The resulting mixture was stirred for additional 3.0 h at 80° C. The mixture was acidified to pH 3-5 with AcOH. The resulting mixture was concentrated under vacuum. The residue was suspended in H₂O (50 mL). The precipitated solids were collected by filtration and washed with H₂O (3×10 mL), dried under vacuum. 8-Isopropyl-2-sulfanylidene-1H,3H-pyrazolo[1,5-a][1,3,5]triazin-4-one (3.00 g, 66.64% yield) was obtained as a white solid. LC/MS: mass calcd. For C₈H₁₀N₄OS: 210.06, found: 211.00 [M+H]⁺.

### Step 6: Synthesis of 8-Isopropyl-2-(methylsulfanyl)-1H-pyrazolo[1,5-a][1,3,5]triazin-4-one

To a stirred solution of 8-isopropyl-2-sulfanylidene-1H,3H-pyrazolo[1,5-a][1,3,5] triazin-4-one (2.50 g, 11.89 mmol, 1.00 equiv) in EtOH (35.00 mL) and 2M NaOH in H₂O (12.00 mL) was added methyl iodide (1.86 g, 13.10 mmol, 1.10 equiv) dropwise at 0° C. The resulting mixture was stirred for 3.0 h at room temperature. The mixture was acidified to pH 3˜5 with 6 M HCl. The resulting mixture was concentrated under vacuum. The residue was suspended in water (20 mL). The precipitated solids were collected by filtration and washed with H₂O (3×5 mL), dried under vacuum. 8-Isopropyl-2-(methylsulfanyl)-1H-pyrazolo[1,5-a][1,3,5]triazin-4-one (1.10 g, 41.25% yield) was obtained as a white solid. LC/MS: mass calcd. For C₉H₁₂N₄OS: 224.07, found: 225.10 [M+H]⁺.

### Step 7: Synthesis of 4-Chloro-8-isopropyl-2-(methylsulfanyl)pyrazolo[1,5-a][1,3,5]triazine

To a stirred solution of 8-isopropyl-2-(methylsulfanyl)-1H-pyrazolo[1,5-a][1,3,5] triazin-4-one (1.00 g, 4.46 mmol, 1.00 equiv) in POCl₃ (10.00 mL) was added N,N-diethylaniline (2.00 g, 13.40 mmol, 3.01 equiv) dropwise at room temperature. The resulting mixture was stirred for 3.0 h at 90° C. The resulting mixture was concentrated under vacuum. 4-Chloro-8-isopropyl-2-(methylsulfanyl)pyrazolo[1,5-a][1,3,5] triazine (1.17 g, crude) was obtained as a red oil. LC/MS: mass calcd. For C₉H₁₁ClN₄S: 242.04, found: 243.05 [M+H]⁺.

### Step 8: N-[[4-(benzyloxy)-2-(pyrazol-1-yl)phenyl]methyl]-8-isopropyl-2-(methylsulfanyl)pyrazolo[1,5-a][1,3,5]triazin-4-amine

To a stirred solution of 4-chloro-8-isopropyl-2-(methylsulfanyl)pyrazolo[1,5-a][1,3,5]triazine (1.00 g, 4.12 mmol, 1.00 equiv) in CH₃CN (15.00 mL) was added 1-[4-(benzyloxy)-2-(pyrazol-1-yl)phenyl]methanamine (1.15 g, 4.12 mmol, 1.00 equiv) and DIEA (2.66 g, 20.60 mmol, 5.00 equiv) in portions at room temperature. The resulting mixture was stirred for 17.0 h at 50° C.

The resulting mixture was concentrated under vacuum. The residue was dissolved in EA (50 mL). The resulting mixture was washed with sat. aq. NaHCO₃ (1×30 mL) and brine (1×20 mL), dried over anhydrous Na₂SO₄. After filtration, the filtrate was concentrated under vacuum.
The residue was purified by silica gel column chromatography, elute with PE/EA (4:1) to afford N-[[4-(benzyloxy)-2-(pyrazol-1-yl)phenyl]methyl]-8-isopropyl-2-(methylsulfanyl)pyrazolo[1,5-a][1,3,5]triazin-4-amine (1.10 g, 53.44% yield) as a yellow solid. LC/MS: mass calcd. For C₂₆H₂₇N₇OS: 485.20, found: 486.30 [M+H]⁺.

### Step 9: Synthesis of N-[[4-(benzyloxy)-2-(pyrazol-1-yl)phenyl]methyl]-8-isopropyl-2-methanesulfonylpyrazolo[1,5-a][1,3,5]triazin-4-amine

To a stirred solution of N-[[4-(benzyloxy)-2-(pyrazol-1-yl)phenyl]methyl]-8-isopropyl-2-(methylsulfanyl)pyrazolo[1,5-a][1,3,5]triazin-4-amine (1.00 g, 2.06 mmol, 1.00 equiv) in DCM (15.00 mL) was added m-CPBA (1.07 g, 6.20 mmol, 3.01 equiv) in portions at room temperature. The resulting mixture was stirred for 3.0 h at room temperature. The resulting mixture was washed with sat. aq. NaHCO₃ (1×10 mL) and brine (1×5 mL), dried by Na₂SO₄. After filtration, the filtrate was concentrated under reduced pressure.

The residue was purified by silica gel column chromatography, eluted with PE/EA (3:2) to afford N-[[4-(benzyloxy)-2-(pyrazol-1-yl)phenyl]methyl]-8-isopropyl-2-methanesulfonylpyrazolo[1,5-a][1,3,5]triazin-4-amine (1.00 g, 58.05% yield) as a colorless oil. LC/MS: mass calcd. For C₂₆H₂₇N₇O₃S: 517.19, found: 540.30 [M+Na]⁺.

### Step 10: Synthesis of tert-butyl 3-[[4-([[4-(benzyloxy)-2-(pyrazol-1-yl)phenyl]methyl]amino)-8-isopropylpyrazolo[1,5-a][1,3,5]triazin-2-yl]oxy]piperidine-1-carboxylate

To a stirred solution of tert-butyl 3-hydroxypiperidine-1-carboxylate (1.17 g, 5.81 mmol, 3.01 equiv) in DMF (10.00 mL) was added 1 M KHMDS in THF (5.80 mL) dropwise at room temperature. The resulting mixture was stirred for 10 min at room temperature. To the above mixture was added N-[[4-(benzyloxy)-2-(pyrazol-1-yl)phenyl]methyl]-8-isopropyl-2-methanesulfonylpyrazolo[1,5-a][1,3,5]triazin-4-amine (1.00 g, 1.93 mmol, 1.00 equiv) in DMF (5.00 mL) dropwise at room temperature. The resulting mixture was stirred for additional 2.0 h at 70° C. The resulting mixture was diluted with EA (60 mL). The mixture was washed with sat. aq. NaHCO₃ (1×30 mL) and brine (3×30 mL), dried over anhydrous Na₂SO₄. After filtration, the filtrate was concentrated under vacuum. The residue was purified by silica gel column chromatography, eluted with PE/EA (3:1) to afford tert-butyl 3-[[4-([[4-(benzyloxy)-2-(pyrazol-1-yl)phenyl]methyl]amino)-8-isopropylpyrazolo[1,5-a][1,3,5]triazin-2-yl]oxy]piperidine-1-carboxylate (900.00 mg, 72.93% yield) as yellow oil. LC/MS: mass calcd. For C₃₅H₄₂N₈O₄: 638.33, found: 639.45 [M+H]⁺.

### Step 11: Tert-butyl 3-[[4-([[4-hydroxy-2-(pyrazol-1-yl)phenyl]methyl]amino)-8-isopropylpyrazolo[1,5-a][1,3,5]triazin-2-yl]oxy]piperidine-1-carboxylate

To a stirred solution of tert-butyl 3-[[4-([[4-(benzyloxy)-2-(pyrazol-1-yl)phenyl]methyl]amino)-8-isopropylpyrazolo[1,5-a][1,3,5]triazin-2-yl]oxy]piperidine-1-carboxylate (900.00 mg, 1.41 mmol, 1.00 equiv) in MeOH (20.00 mL) was added Pd/C (149.94 mg) in portions at room temperature. Then H₂ was exchanged by three times.

The resulting mixture was stirred for 17.0 h at room temperature. The resulting mixture was filtered, the filter cake was washed with EA (3×10 mL). The filtrate was concentrated under vacuum. Tert-butyl 3-[[4-([[4-hydroxy-2-(pyrazol-1-yl)phenyl] methyl]amino)-8-isopropylpyrazolo[1,5-a][1,3,5]triazin-2-yl]oxy]piperidine-1-carboxylate (720.00 mg, 91.90% yield) was obtained as a white solid. LC/MS: mass calcd. For C₂₈H₃₆N₈O₄: 548.29, found: 549.40 [M+H]⁺.

### Example 7. Synthesis of 2-[([3-ethyl-5-[(2S)-2-[2-(oxan-2-yloxy)ethyl]piperidin-1-yl]pyrazolo[1,5-a]pyrimidin-7-yl]amino)methyl]-3-(oxan-2-yl)-1,3-benzodiazol-5-ol (INT-23-THP)

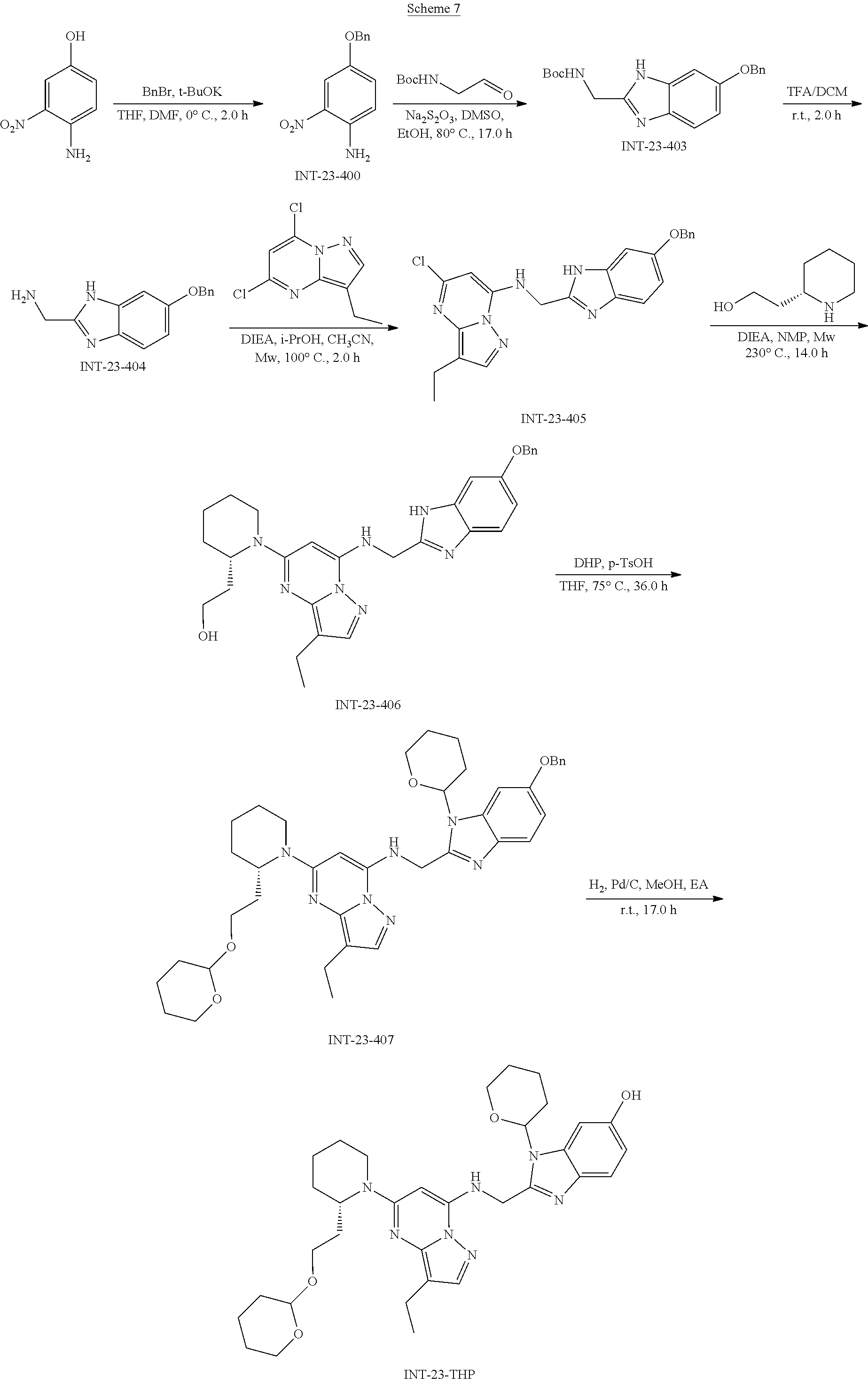

### Step 1: Synthesis of 4-(benzyloxy)-2-nitroaniline

To a solution of 4-amino-3-nitrophenol (4.00 g, 25.95 mmol, 1.00 equiv) in DMF (30.00 mL) was added the solution of t-BuOK (3.20 g, 28.25 mmol, 1.10 equiv) in THF (7.00 mL) over 10 min at 0° C. After the mixture had been stirred for 20 min at 0° C., the solution of benzyl bromide (4.88 g, 28.25 mmol, 1.10 equiv) in DMF (5.00 mL) was added dropwise. The reaction mixture was stirred at 0° C. for 2.0 h. Then 100 mL NH₄Cl was added dropwise to quench the reaction. The product was filtered out and washed with water until the filtration become colorless. The filter cake was dried under vacuum. 6.29 g of desired product was obtained as red solid (99.33% yield). LC/MS: mass calcd. For C₁₃H₁₂N₂O₃: 244.08, found: 245.20 [M+H]⁺.

### Step 2: Synthesis of tert-butyl N-[[5-(benzyloxy)-3H-1,3-benzodiazol-2-yl]methyl] carbamate

Into a sealed tube was added 4-(benzyloxy)-2-nitroaniline (2.80 g, 11.46 mmol, 1.00 equiv), tert-butyl N-(2-oxoethyl)carbamate (2.19 g, 13.76 mmol, 1.20 equiv), Na₂S₂O₄ (5.99 g, 34.39 mmol, 3.00 equiv), EtOH (20.00 mL) and DMSO (40.00 mL). The mixture was stirred at 80° C. for 17.0 h. Then the mixture was poured into 50 mL ice water, EA extracted (3×50 mL). The organic phases were combined and washed with water (50 mL), brine (50 mL), then dried over anhydrous Na₂SO₄. The solid was filtered out and concentrated. The residue was purified by silica gel column with PE/EA=1:1. 1.71 g desired product was obtained as dark yellow solid. (38.00% yield). LC/MS: mass calcd. For C₂₀H₂₃N₃O₃: 353.17, found: 354.25 [M+H]⁺.

### Step 3: Synthesis of 1-[5-(benzyloxy)-3H-1,3-benzodiazol-2-yl]methanamine trifluoroacetic acid salt

To a solution of tert-butyl N-[[5-(benzyloxy)-3H-1,3-benzodiazol-2-yl]methyl] carbamate (1.71 g, 4.84 mmol, 1.00 equiv) in DCM (25.00 mL) was added TFA (5.00 mL). The mixture was stirred at r.t. for 2.0 h. Then the solvent was removed and the residue was used for the next step directly. 1.71 g crude product was obtained as TFA salt and it was a dark yellow solid. LC/MS: mass calcd. For C₁₅H₁₅N₃O: 253.12, found: 254.15 [M+H]⁺.

### Step 4: N-[[5-(benzyloxy)-3H-1,3-benzodiazol-2-yl]methyl]-5-chloro-3-ethylpyrazolo[1,5-a]pyrimidin-7-amine

Into a microwave tube was added 1-[5-(benzyloxy)-3H-1,3-benzodiazol-2-yl] methanamine trifluoroacetic acid salt (1.71 g, 4.66 mmol, 1.00 equiv), 5,7-dichloro-3-ethylpyrazolo[1,5-a]pyrimidine (1.01 g, 4.66 mmol, 1.00 equiv), i-PrOH (20.00 mL) and CH₃CN (5.00 mL). DIEA (3.01 g, 23.29 mmol, 5.00 equiv) was added at last. The solution was heated to 100° C. under the microwave condition and stirred for 2.0 h. The solvent was removed and the residue was purified by silica gel column with 100% EA. 2.00 g desired product was obtained as dark yellow solid (89.00% yield). LC/MS: mass calcd. For C₂₃H₂₁ClN₆O: 432.15, found: 433.20 [M+H]⁺.

### Step 5: Synthesis of 2-[(2S)-1-[7-([[5-(benzyloxy)-3H-1,3-benzodiazol-2-yl]methyl] amino)-3-ethylpyrazolo [1,5-a]pyrimidin-5-yl]piperidin-2-yl]ethanol

Into a microwave tube was added N-[[5-(benzyloxy)-3H-1,3-benzodiazol-2-yl] methyl]-5-chloro-3-ethylpyrazolo[1,5-a]pyrimidin-7-amine (2.00 g, 4.62 mmol, 1.00 equiv), 2-[(2S)-piperidin-2-yl]ethanol (1.79 mol, 13.86 mmol, 3.00 equiv), NMP (28.00 mL) and DIEA (2985.45 mg, 23.10 mmol, 5.00 equiv). The mixture was stirred at 230° C. for 14.0 h under the microwave condition. Then the mixture was poured into 100 mL ice water, EA extracted (3×70 mL). The organic phases were combined, washed with water (3×50 mL) and dried over anhydrous Na₂SO₄. The solid was filtered out and concentrated. The residue was purified by silica gel column with DCM/MeOH=12:1. 1.01 g desired product was obtained as dark yellow solid (37.00% yield). LC/MS: mass calcd. For C₃₀H₃₅N₇O₂: 525.29, found: 526.40 [M+H]⁺.

### Step 6: Synthesis of N-[[6-(benzyloxy)-1-(oxan-2-yl)-1,3-benzodiazol-2-yl]methyl]-3-ethyl-5-[(2S)-2-[2-(oxan-2-yloxy)ethyl]piperidin-1-yl]pyrazolo[1,5-a]pyrimidin-7-amine

To a solution of 2-[(2S)-1-[7-([[5-(benzyloxy)-3H-1,3-benzodiazol-2-yl]methyl] amino)-3-ethylpyrazolo [1,5-a]pyrimidin-5-yl]piperidin-2-yl]ethanol (1.01 g, 1.92 mmol, 1.00 equiv) in THF (10.00 mL) was added DHP (3.22 g, 38.35 mmol, 20.00 equiv) and p-TsOH (66.04 mg, 0.384 mmol, 0.20 equiv). The mixture was stirred at 75° C. for 36.0 h under N₂ atmosphere. Then the solvent was removed and the residue was purified by reveres phase column under the condition: H₂O (0.05% NH₄HCO₃)/CH₃CN, form 20% to 90% in 100 min. The fractions were combined and concentrated. 600.00 mg desired product was obtained as light brown solid (42.39% yield). LC/MS: mass calcd. For C₄₀H₅₁N₇O₄: 693.40, found: 694.55 [M+H]⁺.

### Step 7: Synthesis of 2-[([3-ethyl-5-[(2S)-2-[2-(oxan-2-yloxy)ethyl]piperidin-1-yl]pyrazolo[1,5-a]pyrimidin-7-yl]amino)methyl]-3-(oxan-2-yl)-1,3-benzodiazol-5-ol

A solution of N-[[6-(benzyloxy)-1-(oxan-2-yl)-1,3-benzodiazol-2-yl]methyl]-3-ethyl-5-[(2S)-2-[2-(oxan-2-yloxy)ethyl]piperidin-1-yl]pyrazolo[1,5-a]pyrimidin-7-amine (500.00 mg, 0.72 mmol, 1.00 equiv) in EA (3.00 mL) and MeOH (3.00 mL) was stirred at r.t. overnight. The mixture was filtrated and concentrated to afford 2-[([3-ethyl-5-[(2S)-2-[2-(oxan-2-yloxy)ethyl]piperidin-1-yl]pyrazolo[1,5-a]pyrimidin-7-yl]amino)methyl]-3-(oxan-2-yl)-1,3-benzodiazol-5-ol (400.00 mg, 91.94% yield). LCMS: mass calcd. For C₃₃H₄₅N₇O₄: 603.35, found: 604.50 [M+H]⁺.

### Example 8. Synthesis of (2S)-2-[(2S)-6-{bicyclo[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoic acid (INT-024-OH)

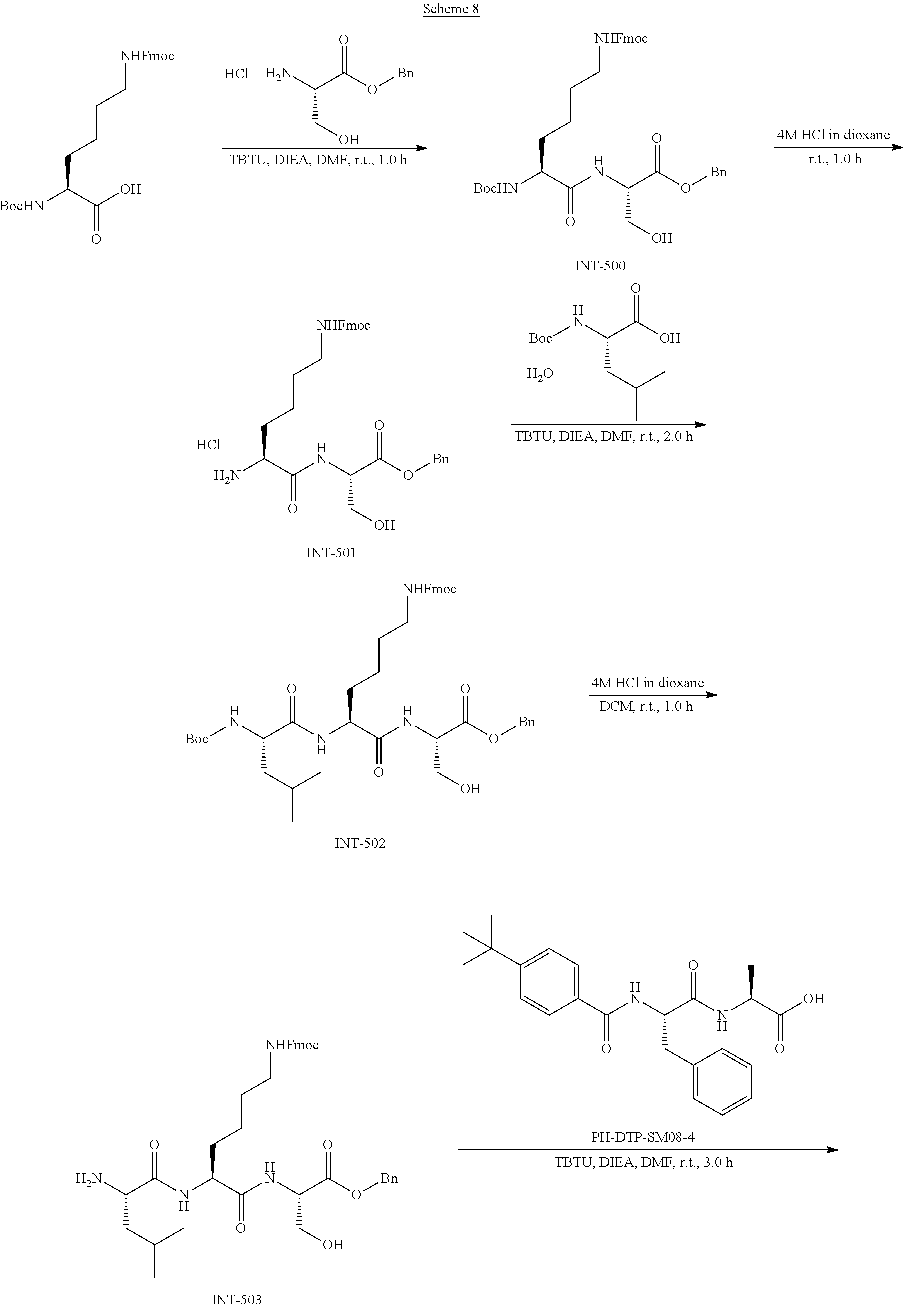

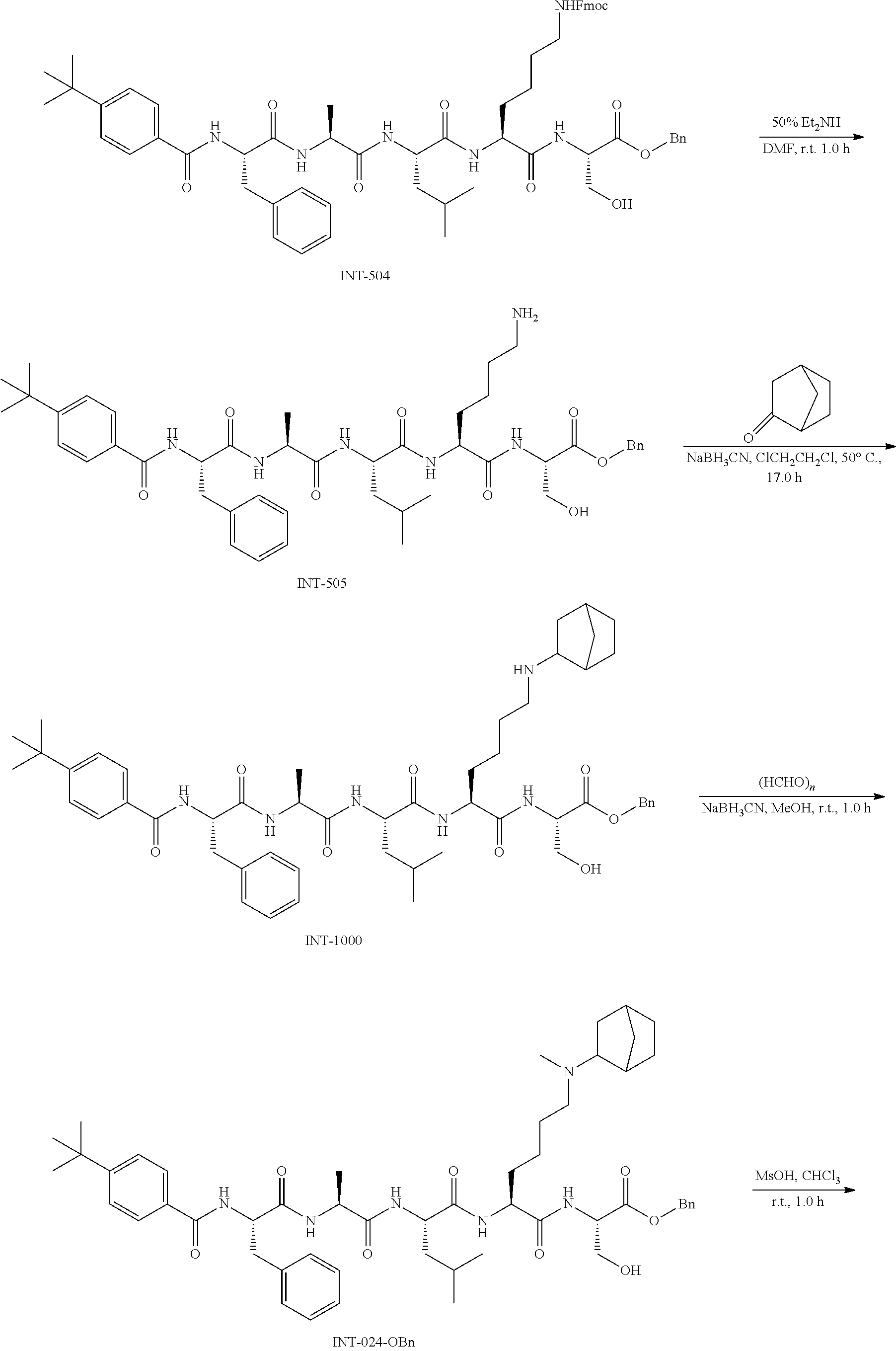

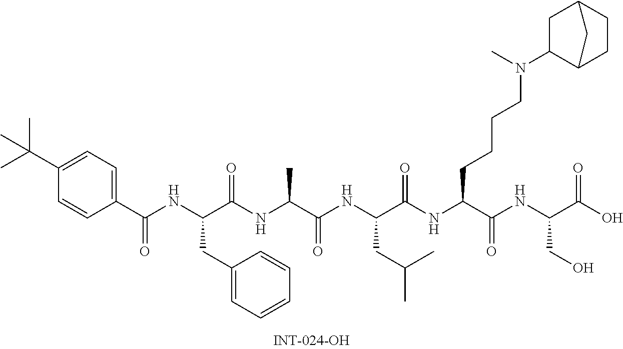 [SMILES: CC(C)C[C@H](NC(=O)[C@H](C)NC(=O)[C@H](Cc1ccccc1)NC(=O)c1ccc(C(C)(C)C)cc1)C(=O)N[C@@H](CCCCN(C)C1CC2CCC1C2)C(=O)N[C@@H](CO)C(=O)O]

### Step 1: Synthesis of benzyl (2S)-2-[(2S)-2-[(tert-butoxycarbonyl)amino]-6-{[(9H-fluoren-9-ylmethoxy)carbonyl]amino}hexanamido]-3-hydroxypropanoate

The procedure was the same as methyl (2S)-2-[(4-tert-butylphenyl)formamido]-3-phenylpropanoate (INT-14.1). 10.00 g of (2S)-2-[(tert-butoxycarbonyl) amino]-6-{[(9H-fluoren-9-ylmethoxy)carbonyl]amino}hexanoic acid was used, 13.00 g of benzyl (2S)-2-[(2S)-2-[(tert-butoxycarbonyl)amino]-6-{[(9H-fluoren-9-ylmethoxy)carbonyl]amino}hexanamido]-3-hydroxypropanoate was obtained as white solid (94.33% yield). LC/MS: mass calcd. For C₃₆H₄₃N₃O₈: 645.31, found: 646.50 [M+H]⁺.

### Step 2: Synthesis of benzyl (2S)-2-[(2S)-2-amino-6-{[(9H-fluoren-9-ylmethoxy) carbonyl]amino}hexanamido]-3-hydroxypropanoate

The procedure was the same as methyl (2S)-2-[(2S)-2-amino-6-{[(9H-fluoren-9-yl)methoxy)carbonyl]amino}hexanamido]-3-hydroxypropanoate (INT-14.6). 9.00 g of benzyl (2S)-2-[(2S)-2-[(tert-butoxy carbonyl)amino]-6-{[(9H-fluoren-9-ylmethoxy)carbonyl]amino}hexanamido]-3-hydroxypropanoate was used, 7.60 g crude of desired product was obtained as white solid. LC/MS: mass calcd. for C₃₁H₃₅N₃O₆: 545.25, found: 546.40 [M+H]⁺.

### Step 3: Synthesis of benzyl (2S)-2-[(2S)-2-[(2S)-2-[(tert-butoxycarbonyl)amino]-4-methylpentanamido]-6-{[(9H-fluoren-9-ylmethoxy)carbonyl]amino}hexanamido]-3-hydroxypropanoate

The procedure was the same as methyl (2S)-2-[(4-tert-butylphenyl)formamido]-3-phenylpropanoate (INT-14.1). 7.60 g of benzyl (2S)-2-[(2S)-2-amino-6-{[(9H-fluoren-9-ylmethoxy)carbonyl]amino}hexanamido]-3-hydroxypropanoate was used, 10.04 g of desired product was obtained as white solid (94.98% yield). LC/MS: mass calcd. for C₄₂H₅₄N₄O₉: 758.38, found: 759.60 [M+H]⁺.

### Step 4: Synthesis of benzyl (2S)-2-[(2S)-2-[(2S)-2-amino-4-methylpentanamido]-6-{[(9H-fluoren-9-ylmethoxy)carbonyl]amino}hexanamido]-3-hydroxypropanoate

The procedure was the same as methyl (2S)-2-[(2S)-2-amino-6-{[(9H-fluoren-9-yl)methoxy)carbonyl]amino}hexanamido]-3-hydroxypropanoate (INT-14.6). 4.32 g of benzyl (2S)-2-[(2S)-2-[(2S)-2-[(tert-butoxycarbonyl)amino]-4-methyl pentanamido]-6-{[(9H-fluoren-9-ylmethoxy)carbonyl]amino}hexanamido]-3-hydroxypropanoate was used, 3.75 g crude of desired product was obtained as yellow solid. LC/MS: mass calcd. For C₃₇H₄₆N₄O₇: 658.34, found: 659.40[M+H]⁺.

### Step 5: Synthesis of benzyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-{[(9H-fluoren-9-ylmethoxy)carbonyl]amino}hexanamido]-3-hydroxypropanoate

To a stirred solution of (2S)-2-[(2S)-2-[(4-tert-butylphenyl)formamido]-3-phenylpropanamido]propanoic acid (4.32 g, 10.90 mmol, 1.00 equiv) in DMF (150.00 mL) was added TBTU (5.25 g, 16.34 mmol, 1.50 equiv), DIEA (4.22 g, 32.69 mmol, 3.00 equiv) and benzyl (2S)-2-[(2S)-2-[(2S)-2-amino-4-methylpentanamido]-6-{[(9H-fluoren-9-ylmethoxy)carbonyl]amino}hexanamido]-3-hydroxypropanoate (7.90 g, 11.99 mmol, 1.10 equiv) in portions at 0° C. The resulting mixture was stirred for 3.0 h at room temperature. The reaction was poured into ice water (450 mL). The precipitated solids were collected by filtration and washed with H₂O (3×150 mL), dried under vacuum. Then the precipitated solids was purified by reverse flash chromatography with the following conditions: column, C18 silica gel; mobile phase, ACN in water (0.05% TFA), 35% to 50% gradient in 20 min; detector, UV 254 n. The fractions were combined and concentrated. Benzyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-{[(9H-fluoren-9-ylmethoxy)carbonyl]amino}hexanamido]-3-hydroxypropanoate (3.20 g, 28.31% yield) was obtained as white solid. Then 1.50 g of it was purified by Achiral-Prep-HPLC under the following condition: Column: GreenSep Basic, 3*15 cm, 5 μm; Mobile Phase A: CO₂, Mobile Phase B: MeOH (0.5% 2M NH3-MeOH)-HPLC; Flow rate: 75 mL/min; Gradient: isocratic 37% B; Column Temperature (° C.): 35; Back Pressure (bar): 100; Wave Length: 254 nm; RT1 (min): 2.95; RT2 (min): 4.05; Sample Solvent: DCM-HPLC; Injection Volume: 1 mL; Number Of Runs: 30. The fractions were combined and concentrated, 700.00 mg of desired product was obtained as light yellow solid. LC/MS: mass calcd. For C₆₀H₇₂N₆O₁₀: 1036.53, found: 1037.95[M+H]⁺.

### Step 6: Synthesis of benzyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate

The procedure was the same as methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-14.11), but the reaction time was 1.0 h. 2.30 g of benzyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-{[(9H-fluoren-9-ylmethoxy)carbonyl]amino}hexanamido]-3-hydroxypropanoate was used, 1.20 g of desired product was obtained as yellow solid. LC/MS: mass calcd. For C₄₅H₆₂N₆O₈: 814.46, found: 815.75[M+H]⁺.

### Step 7: Synthesis of benzyl (2S)-2-[(2S)-6-{bicyclo[2.2.1]heptan-2-ylamino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate

To a stirred solution of benzyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (500.00 mg, 0.61 mmol, 1.00 equiv) in ClCH₂CH₂Cl (10.00 mL) was added NaBH₃CN (308.42 mg, 4.90 mmol, 8.00 equiv) and in portions at 0° C. The resulting mixture was stirred for 17.0 h at 50° C. The reaction mixture was diluted with NH₄Cl (5 mL) and concentrated and the residue was dissolved in DMF (5.0 mL) and purified by reverse flash chromatography with the following conditions: column, C18 silica gel; mobile phase, ACN in water (0.05% TFA), 30% to 50% gradient in 20 min; detector, UV 254 nm. The fractions were combined and concentrated. Benzyl (2S)-2-[(2S)-6-{bicyclo[2.2.1]heptan-2-ylamino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (280.00 mg, 50.20% yield) was obtained as white solid. LC/MS: mass calcd. For C₅₂H₇₂N₆O₈: 908.54, found: 455.45[1/2M+H]⁺.

### Step 8: Synthesis of benzyl (2S)-2-[(2S)-6-{bicyclo[2.2.1]heptan-2-yl(methyl) amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate

To a stirred solution of benzyl (2S)-2-[(2S)-6-{bicyclo[2.2.1]heptan-2-ylamino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (200.00 mg, 0.220 mmol, 1.00 equiv) in MeOH (6 mL) was added NaBH₃CN (110.59 mg, 1.76 mmol, 8.00 equiv) and (HCHO)ₙ (329.97 mg, 1.10 mmol, 5.00 equiv) in portions at room temperature. The resulting mixture was stirred for 1.0 h at room temperature. The reaction mixture was diluted with NH₄Cl (20.00 mL), then the resulting mixture was concentrated under vacuum. The reaction mixture was purified by reverse flash chromatography with the following conditions: column, C18 silica gel; mobile phase, ACN in water (0.05% TFA), 30% to 35% gradient in 20 min; detector, UV 254 n. The fractions were combined and concentrated. Benzyl (2S)-2-[(2S)-6-{bicyclo[2.2.1] heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (200.00 mg, 98.48% yield) was obtained as white solid. LC/MS: mass calcd. For C₅₃H₇₄N₆O: 922.56, found: 923.60[M+H]⁺.

### Step 9: Synthesis of (2S)-2-[(2S)-6-{bicyclo[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoic acid

To a stirred solution of benzyl (2S)-2-[(2S)-6-{bicyclo[2.2.1]heptan-2-yl(methyl) amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (450.00 mg, 0.49 mmol, 1.00 equiv) in CHCl₃ (5.00 mL) was added CH₃SO₃H (1.00 mL) dropwise at room temperature. The resulting mixture was stirred for 1.0 h at room temperature. The resulting mixture was concentrated under vacuum. The reaction mixture was purified by reverse flash chromatography with the following conditions: column, C18 silica gel; mobile phase, ACN in water (0.05% TFA), 25% to 30% gradient in 15 min; detector, UV 254 nm. The fractions were combined and concentrated. (2S)-2-[(2S)-6-{bicyclo[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoic acid (220.00 mg, 54.18% yield) was obtained as yellow oil. LC/MS: mass calcd. For C₄₆H₆₈N₆O:832.51, found: 833.40[M+H]⁺

### Example 9. Synthesis of 4-{5-[(furan-2-ylmethyl)amino]-[1,2,4]triazolo[4,3-c]pyrimidin-8-yl}benzoic acid (INT-25)

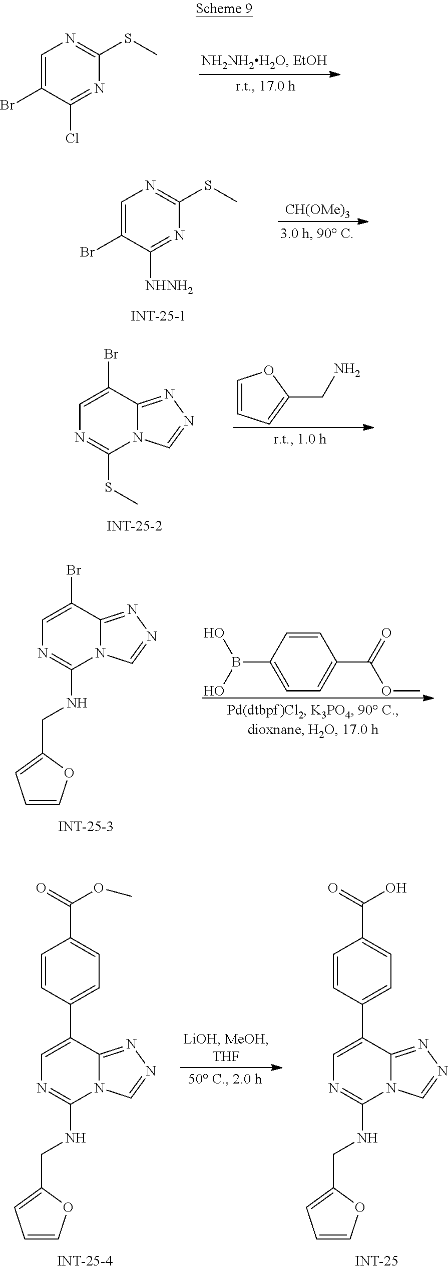

### Step 1: Synthesis of 5-bromo-4-hydrazinyl-2-(methylsulfanyl)pyrimidine

Into a 250 ml flask was added 5-bromo-4-chloro-2-(methylsulfanyl)pyrimidine (9.00 g, 37.58 mmol, 1.00 equiv), EtOH (100.00 mL), NH₂NH₂·H₂O (3.78 g, 75.53 mmol, 2.01 equiv), the reaction was stirred at r.t. for 17.0 h. The precipitated solids were collected by filtration and washed with hexane (3×10 mL), dried under vacuum. This resulted in 5-bromo-4-hydrazinyl-2-(methylsulfanyl)pyrimidine (10.00 g, crude) as a white solid. LC/MS: mass calcd. For C₅H₇BrN₄S:233.96, found: 235.00, 237.00 [M+H, M+2+H]⁺.

### Step 2: Synthesis of 8-bromo-5-(methylsulfanyl)-[1,2,4]triazolo[4,3-c]pyrimidine

Into a 100 mL flask were added 5-bromo-4-hydrazinyl-2-(methylsulfanyl) pyrimidine (5.00 g, 0.02 mmol, 1.00 equiv) and CH(OMe)₃ (40.00 mL). The mixture was stirred for 3.0 h at 90° C. The precipitated solids were collected by filtration and washed with ethanol (3×50 mL). This result in 8-bromo-5-(methylsulfanyl)-[1,2,4]triazolo[4,3-c]pyrimidine (4.20 g, 81.00% yield) as an orange solid. LC/MS: mass calcd. For C₆H₅BrN₄S:243.94, found: 244.95, 246.95 [M+H, M+H+2]⁺.

### Step 3: 8-bromo-N-(furan-2-ylmethyl)-[1,2,4]triazolo[4,3-c]pyrimidin-5-amine

Into a 25 mL flask were added 8-bromo-5-(methylsulfanyl)-[1,2,4]triazolo[4,3-c] pyrimidine (500.00 mg, 2.05 mmol, 1.00 equiv) and furylamine (5.00 mL). The reaction was stirred for 1.0 h at r.t. The precipitated solids were collected by filtration and washed with ethanol (3×50 mL). This result in 8-bromo-N-(furan-2-ylmethyl)-[1,2,4]triazolo[4,3-c]pyrimidin-5-amine (435.00 mg, 73.00% yield) as a white solid. LC/MS: mass calcd. For C₁₀H₈BrN₅O: 292.99, found: 293.85, 295.85 [M+H, M+2+H]⁺.

### Step 4: methyl 4-{5-[(furan-2-ylmethyl)amino]-[1,2,4]triazolo[4,3-c]pyrimidin-8-yl}benzoate

Into a 100 mL flask were added 8-bromo-N-(furan-2-ylmethyl)-[1,2,4] triazolo[4,3-c]pyrimidin-5-amine (500.00 mg, 1.70 mmol, 1.00 equiv) in dioxane (20.00 mL) and H₂O (2.00 mL). Then K₃PO₄ (1.08 g, 5.10 mmol, 3.00 equiv) and Pd(DtBPF)Cl₂ (554.00 mg, 0.85 mmol, 0.50 equiv) were added to the above mixture. The mixture was heated to at 90° C. under N₂ overnight. The resulting mixture was cooled to room temperature, and 20 mL H₂O was added to the mixture and extracted with EtOAc (3×20 mL). The combined organic layers were dried by anhydrous Na₂SO₄, filtered, and concentrated. The residue was purified by silica gel column chromatography, eluted with CH₂Cl₂/MeOH (9:1) to afford methyl 4-{5-[(furan-2-ylmethyl)amino]-[1,2,4]triazolo[4,3-c]pyrimidin-8-yl}benzoate (600.00 mg, 96.91% yield) as a yellow solid. LC/MS: mass calcd. For C₁₈H₁₁₅N₅O₃:349.12, found: 350.15 [M+H]⁺.

### Step 5: Synthesis of 4-{5-[(furan-2-ylmethyl)amino]-[1,2,4]triazolo[4,3-c]pyrimidin-8-yl}benzoic acid

To a stirred solution of methyl 4-{5-[(furan-2-ylmethyl)amino]-[1,2,4]triazolo [4,3-c]pyrimidin-8-yl}benzoate (600.00 mg, 1.72 mmol, 1.00 equiv) in MeOH (10.00 mL) and THF (10.00 mL) was added LiOH solution (2M, 8.60 mL, 17.19 mmol, 10.00 equiv) dropwise at room temperature. The resulting mixture was stirred for 2.0 h at 50° C. The resulting mixture was concentrated under reduced pressure.

The residue was dissolved in H₂O (5 mL). The mixture was acidified to pH 3-5 with 2 M HCl. The precipitated solids were collected by filtration and washed with H₂O (3×10 mL), dried under vacuum. 4-{5-[(furan-2-ylmethyl)amino]-[1,2,4]triazolo[4,3-c] pyrimidin-8-yl}benzoic acid (450.00 mg, 78.00% yield) was obtained as a white solid. LC/MS: mass calcd. For C₁₇H₁₃N₅O₃:335.10, found: 336.10[M+H]⁺.

### Example 10. Synthesis of 3-[5-(5-{[(5-fluoro-2,3-dihydro-1-benzofuran-4-yl)methyl]amino}-[1,2,4]triazolo[4,3-c]pyrimidin-8-yl)-6-methylpyridin-2-yl]propanoic acid (SM-040)

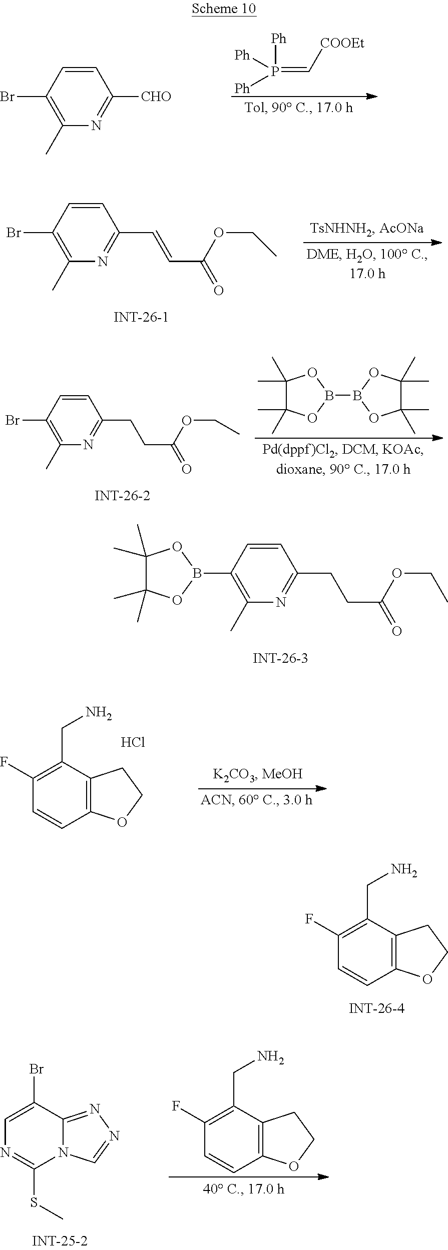

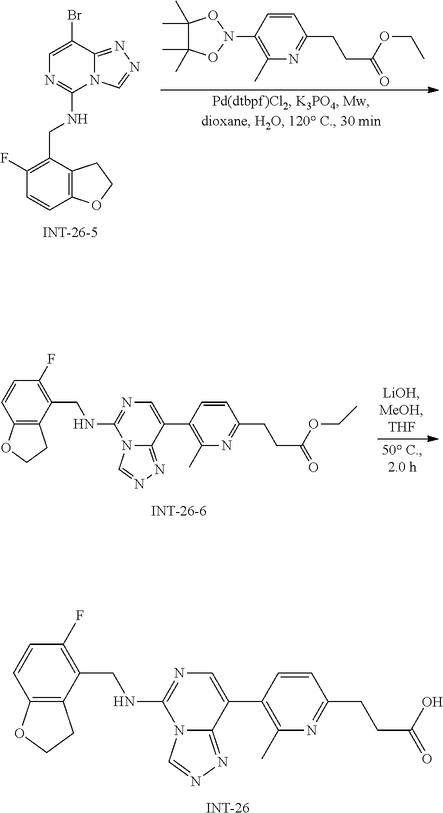

### Step 1: Synthesis of ethyl (2E)-3-(5-bromo-6-methylpyridin-2-yl)prop-2-enoate

Into a 100 mL flask was added 5-bromo-6-methylpyridine-2-carbaldehyde (1.00 g, 5.02 mmol, 1.00 equiv) in toluene (40.00 mL) and ethyl 2-(triphenyl-lambda5-phosphanylidene)acetate (2.62 g, 7.53 mmol, 1.50 equiv). The mixture was stirred for 17.0 h at 90° C. The reaction was concentrated under vacuum. The residue was purified by silica gel column chromatography, eluted with PE/EA (25/75) to afford to afford ethyl (2E)-3-(5-bromo-6-methylpyridin-2-yl)prop-2-enoate (1.20 g, 88.90% yield) as a light yellow crystalline. LC/MS: mass calcd. For C₁₁H₁₂BrNO₂:269.01, found: 269.85, 271.85 [M+H, M+2+H]⁺.

### Step 2: ethyl 3-(5-bromo-6-methylpyridin-2-yl)propanoate

Into a 100 mL flask were added ethyl (2E)-3-(5-bromo-6-methylpyridin-2-yl)prop-2-enoate (1.20 g, 4.46 mmol, 1.00 equiv), TsNHNH₂ (4.14 g, 22.21 mmol, 5.00 equiv), AcONa (1.09 g, 13.33 mmol, 3.00 equiv) in DME (24.00 mL) and water (6.00 mL). The mixture was stirred for 17.0 h at 100° C. The mixture was concentrated under vacuum and The residue was purified by silica gel chromatography, eluted with PE/EA (9:1) to afford ethyl 3-(5-bromo-6-methylpyridin-2-yl)propanoate (1.14 g, 95%) as a colorless liquid. LC/MS: mass calcd. For C₁₁H₁₄BrNO₂:271.02, found: 271.85, 273.85 [M+H, M+2+H]⁺.

### Step 3: Synthesis of ethyl 3-[6-methyl-5-(4,4,5,5-tetramethyl-1,3,2-dioxaborolan-2-yl)pyridin-2-yl]propanoate

Into a 25 mL flask were added ethyl 3-(5-bromo-6-methylpyridin-2-yl) propanoate (900.00 mg, 3.31 mmol, 1.00 equiv), bis(pinacolato)diboron (2.52 g, 9.92 mmol, 3.00 equiv), AcOK (973.70 mg, 9.92 mmol, 3.00 equiv), Pd(dppf)Cl₂·CH₂Cl₂ (538.81 mg, 0.66 mmol, 0.20 equiv) and 1,4-dioxane (10.00 mL) at r.t. The reaction was stirred for 17.0 h at 90° C. under N₂ atmosphere. The mixture was extracted by EA (3×30 mL), the organic phases were combined and washed by dried over anhydrous Na₂SO₄. After filtration, the filtrate was concentrated under reduced pressure. This crude product (2.12 g crude) was used in the next step directly without further purification. LC/MS: mass calcd. For C₁₇H₂₆BNO₄: 319.20, found: 320.20 [M+H]⁺.

### Step 4: Synthesis of 1-(5-fluoro-2,3-dihydro-1-benzofuran-4-yl)methanamine

Into a 100 mL flask was added 1-(5-fluoro-2,3-dihydro-1-benzofuran-4-yl) methanamine hydrochloride (1.00 g, 4.93 mmol, 1.00 equiv) in MeOH (2.00 mL) and MeCN (20.00 mL), and K₂CO₃ (3.39 g, 24.50 mmol, 5.00 equiv). The mixture was stirred for 3.0 h at 60° C. The mixture was cooled to r.t. The reaction mixture was filtered and the filtrate was concentrated. The residue was purified by silica gel column chromatography, eluted with DCM/MeOH (9/1). The fractions were combined and concentrated to afford 1-(5-fluoro-2,3-dihydro-1-benzofuran-4-yl)methanamine (513.00 mg, 62.50% yield) as an orange solid. LC/MS: mass calcd. For C₉H₁₀FNO:167.07, found: 168.05[M+H]⁺.

### Step 5: Synthesis of 8-bromo-N-[(5-fluoro-2,3-dihydro-1-benzofuran-4-yl) methyl]-[1,2,4]triazolo[4,3-c]pyrimidin-5-amine

Into a 25 mL flask were added 8-bromo-5-(methylsulfanyl)-[1,2,4]triazolo [4,3-c]pyrimidine (372.00 mg, 1.52 mmol, 1.00 equiv) and 1-(5-fluoro-2,3-dihydro-1-benzofuran-4-yl)methanamine (507.48 mg, 3.04 mmol, 2.00 equiv). The mixture was stirred for 17.0 h at 40° C. The reaction was diluted with EtOAc. The precipitated solids were collected by filtration and washed with EtOAc (3×10 mL). This resulted in 8-bromo-N-[(5-fluoro-2,3-dihydro-1-benzofuran-4-yl) methyl]-[1,2,4] triazolo[4,3-c]pyrimidin-5-amine (430.00 mg, 72.27% yield) as a white solid. LC/MS: mass calcd. For C₁₄H₁₁BrFN₅O:363.01, found: 364.00, 366.00 [M+H, M+2+H]⁺.

### Step 6: Synthesis of ethyl 3-[5-(5-{[(5-fluoro-2,3-dihydro-1-benzofuran-4-yl) methyl]amino}-[1,2,4]triazolo[4,3-c]pyrimidin-8-yl)-6-methylpyridin-2-yl]propanoate

8-bromo-N-[(5-fluoro-2,3-dihydro-1-benzofuran-4-yl)methyl]-[1,2,4]triazolo[4,3-c]pyrimidin-5-amine (677.80 mg, 1.81 mmol, 1.80 equiv), ethyl 3-[6-methyl-5-(4,4,5,5-tetramethyl-1,3,2-dioxaborolan-2-yl)pyridin-2-yl]propanoate (330.00 mg, 1.03 mmol, 1.00 equiv), K₃PO₄ (658.46 mg, 3.10 mmol, 3.00 equiv), Pd(DtBPF)Cl₂ (134.78 mg, 0.21 mmol, 0.20 equiv), dioxane (10.00 mL) and H₂O (2.00 mL) were added to the microwave tube. Then N2 exchanged three times. The mixture was heated to 120° C. for 0.5 h under microwave. 10 mL water was added and the resulting mixture was extracted with EtOAc (3×10 mL). The combined organic layers were washed with EtOAc (3×30 mL), dried over anhydrous Na₂SO₄. After filtration, the filtrate was concentrated under reduced pressure. The residue was purified by reverse phase flash with the following conditions (C18 column; mobile phase, ACN in water (0.05% NH₄HCO₃), 10% to 50% gradient in 10 min; detector, UV 254 n). The fraction were combined and concentrated. Ethyl 3-[5-(5-{[(5-fluoro-2,3-dihydro-1-benzofuran-4-yl)methyl]amino}-[1,2,4]triazolo[4,3-c]pyrimidin-8-yl)-6-methylpyridin-2-yl]propanoate (120.00 mg, 24.3% yield) was obtained as a white solid. LC/MS: mass calcd. For C₂₅H₂₅FN₆O₃:476.20, found: 477.20 [M+H]⁺.

### Step 7: 3-[5-(5-{[(5-fluoro-2,3-dihydro-1-benzofuran-4-yl)methyl]amino}-[1,2,4] triazolo[4,3-c]pyrimidin-8-yl)-6-methylpyridin-2-yl]propanoic acid

Into a 50 mL flask were added ethyl 3-[5-(5-{[(5-fluoro-2,3-dihydro-1-benzofuran-4-yl)methyl]amino}-[1,2,4]triazolo[4,3-c]pyrimidin-8-yl)-6-methylpyridin-2-yl]propanoate (120.00 mg, 0.25 mmol, 1.00 equiv), MeOH (2.00 mL) and THF (2.00 mL). Then LiOH solution (2M, 1.26 mL, 2.52 mmol, 10.00 equiv) was added to the above mixture in portions. The reaction was stirred for 2.0 h at 50° C. The resulting mixture was concentrated under reduced pressure. The residue was dissolved in H₂O (30 mL). The mixture was acidified to pH 3˜5 with 2 M HCl. The precipitated solids were collected by filtration and washed with H₂O (3×3 mL), dried under vacuum. This resulted in 3-[5-(5-{[(5-fluoro-2,3-dihydro-1-benzofuran-4-yl)methyl] amino}-[1,2,4]triazolo[4,3-c]pyrimidin-8-yl)-6-methylpyridin-2-yl]propanoic acid (100.00 mg, 86.54% yield) as a white solid. LC/MS: mass calcd. For C₂₃H₂FN₆O₃:448.17, found: 449.15 [M+H]⁺

### Example 11. Synthesis of (2S)-2-[(2S)-2-[2-(2-{2-[(4-tert-butylphenyl)formamido]ethoxy}ethoxy)acetamido]-6-(diethylamino)hexanamido]-3-hydroxypropanoic acid (INT-29-PEG2-C(O)-PTBPh)

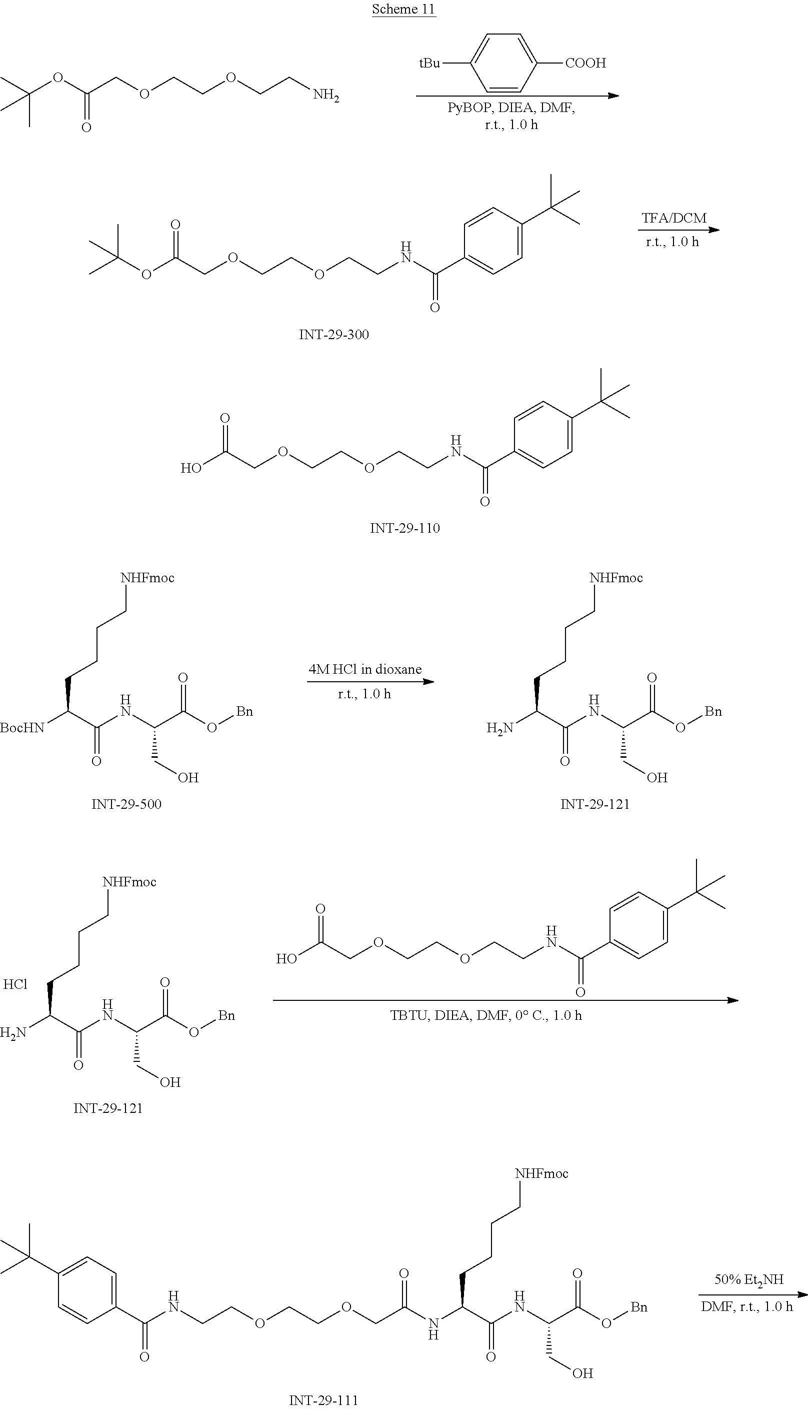

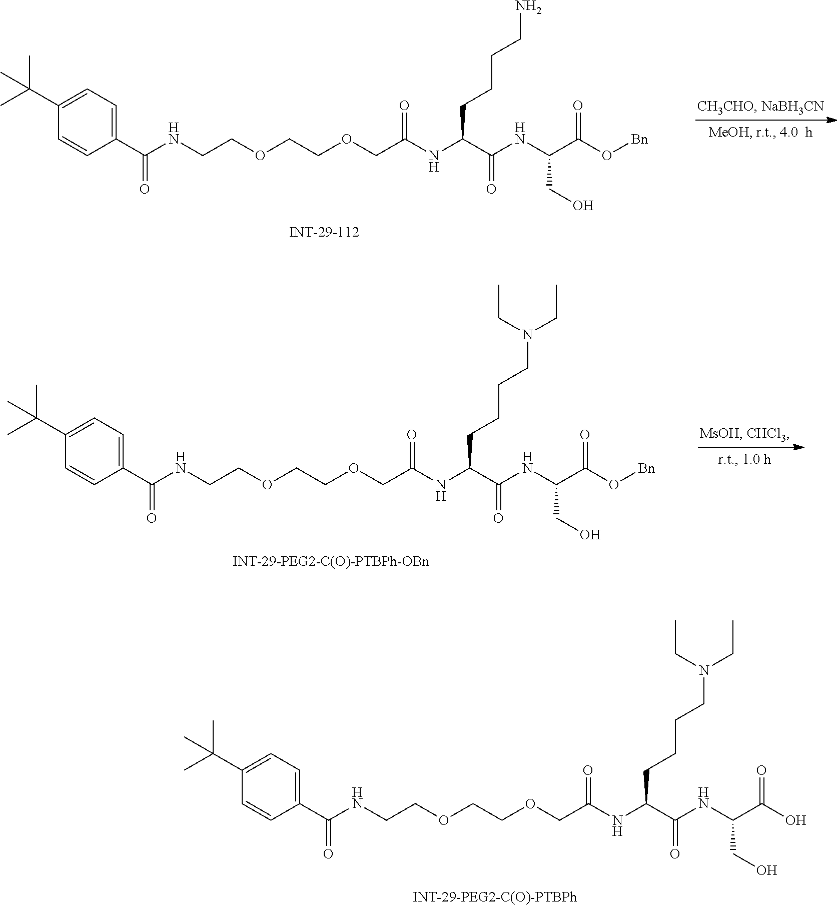 [SMILES: CC(C)(C)c1ccc(C(=O)NCCOCCOCC(=O)N[C@@H](CCCCN)C(=O)N[C@@H](CO)C(=O)OCc2ccccc2)cc1 ; CCN(CC)CCCC[C@H](NC(=O)COCCOCCNC(=O)c1ccc(C(C)(C)C)cc1)C(=O)N[C@@H](CO)C(=O)OCc1ccccc1 ; CCN(CC)CCCC[C@H](NC(=O)COCCOCCNC(=O)c1ccc(C(C)(C)C)cc1)C(=O)N[C@@H](CO)C(=O)O]

### Step 1: Synthesis of tert-butyl 2-(2-{2-[(4-tert-butylphenyl)formamido]ethoxy} ethoxy)acetate

Into a 100 mL flask was added 4-tert-butylbenzoic acid (1.63 g, 9.12 mmol, 1.00 equiv) and DMF (20.00 mL). The mixture was cooled to 0° C., then PyBOP (7.12 g, 13.68 mmol, 1.50 equiv) and tert-butyl 2-[2-(2-aminoethoxy)ethoxy]acetate (2.00 g, 9.12 mmol, 1.00 equiv) were added followed by addition of DIEA (4.77 mL, 27.36 mmol, 3.00 equiv). The reaction was stirred at room temperature for 1.0 h. The reaction mixture was poured into water (60 mL). The reaction mixture was extracted by EA (3×60 mL), the organic phases were combined and washed by H₂O (1×60 mL) and NaCl (1×60 mL), dried over anhydrous Na₂SO₄. After filtration, the filtrate was concentrated under reduced pressure. The residue was purified by silica gel column chromatography, eluted with PE/EtOAc (40:60) to afford tert-butyl 2-(2-{2-[(4-tert-butylphenyl)formamido]ethoxy}ethoxy)acetate (1.18 g, 32.05% yield) as a brown oil. LC/MS: mass calcd. For C₂₁H₃₃NO₅:379.24, found: 380.25 [M+H]⁺.

### Step 2: Synthesis of (2-{2-[(4-tert-butylphenyl)formamido]ethoxy}ethoxy)acetic acid

Into a 50 mL flask were added tert-butyl 2-(2-{2-[(4-tert-butylphenyl) formamido]ethoxy}ethoxy)acetate (1.18 g, 3.11 mmol, 1.00 equiv), DCM (10.00 mL) and TFA (5.00 mL). The mixture was stirred for 1.0 h at r.t. The mixture was concentrated under vacuum and this resulted in (2-{2-[(4-tert-butylphenyl)formamido] ethoxy}ethoxy)acetic acid (1.92 g crude) as white solid. LC/MS: mass calcd. For C₁₇H₂₅NO₅: 323.17, found: 324.10 [M+H]⁺.

### Step 3: Synthesis of Benzyl (2S)-2-[(2S)-2-[2-(2-{2-[(4-tert-butylphenyl) formamido]ethoxy}ethoxy) acetamido]-6-{[(9H-fluoren-9-ylmethoxy)carbonyl] amino}hexanamido]-3-hydroxypropanoate

The procedure was the same as methyl (2S)-2-[(4-tert-butylphenyl)formamido]-3-phenylpropanoate (INT-14.1). 1.36 g of benzyl (2S)-2-[(2S)-2-amino-6-{[(9H-fluoren-9-ylmethoxy)carbonyl]amino}hexanamido]-3-hydroxypropanoate was used, 1.97 g of desired product was obtained as off-white solid. LC/MS: mass calcd. For C₄₈H₅₈N₄O₁₀:850.42, found: 851.40 [M+H]⁺.

### Step 4: Synthesis of benzyl (2S)-2-[(2S)-6-amino-2-[2-(2-{2-[(4-tert-butylphenyl) formamido]ethoxy}ethoxy)acetamido]hexanamido]-3-hydroxypropanoate

The procedure was the same as methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-14.11), but the ratio of Et₂NH and DMF was 1:1. 1.97 g of benzyl (2S)-2-[(2S)-2-[2-(2-{2-[(4-tert-butylphenyl)formamido]ethoxy}ethoxy)acetamido]-6-{[(9H-fluoren-9-ylmethoxy)carbonyl]amino}hexanamido]-3-hydroxypropanoate was used, 1.24 g of desired product was obtained as brown oil (79.40% yield). LC/MS: mass calcd. For C₃₃H₄₈N₄O₈:628.35, found: 629.35[M+H]⁺.

### Step 5: Synthesis of benzyl (2S)-2-[(2S)-2-[2-(2-{2-[(4-tert-butylphenyl) formamido]ethoxy}ethoxy)acetamido]-6-(diethylamino)hexanamido]-3-hydroxypropanoate trifluoroacetic acid

The procedure was the same as benzyl (2S)-2-[(2S)-6-{bicycle[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-024-OBn). 500.00 mg of benzyl (2S)-2-[(2S)-6-amino-2-[2-(2-{2-[(4-tert-butylphenyl)formamido]ethoxy}ethoxy)acetamido]hexanamido]-3-hydroxypropanoate was used, 523.00 mg of desired product was obtained as brown solid (79.56% yield). LC/MS: mass calcd. For C₃₇H₅₆N₄O₈:684.41, found: 685.40 [M+H]⁺.

### Step 6: Synthesis of (2S)-2-[(2S)-2-[2-(2-{2-[(4-tert-butylphenyl)formamido] ethoxy}ethoxy)acetamido]-6-(diethylamino)hexanamido]-3-hydroxypropanoic acid

The procedure was the same as (2S)-2-[(2S)-6-{bicycle[2.2.1]heptan-2-yl (methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoic acid (INT-024-OH). 200.00 mg of benzyl (2S)-2-[(2S)-2-[2-(2-{2-[(4-tert-butylphenyl)formamido]ethoxy}ethoxy)acetamido]-6-(diethylamino)hexanamido]-3-hydroxypropanoate was used, 130.00 mg of desired product was obtained as brown yellow oil. LC/MS: mass calcd. For C₃₀H₅₀N₄O₈:594.36, found: 595.40 [M+H]⁺.

### Example 12. Synthesis of (2S)-2-[3-(2-{[(4-tert-butylphenyl)formamido]methyl}-3H-imidazol-4-yl)azetidine-1-carbonylamino]-6-(diethylamino)hexanoic acid (INT-36-UAZ-IMTB)

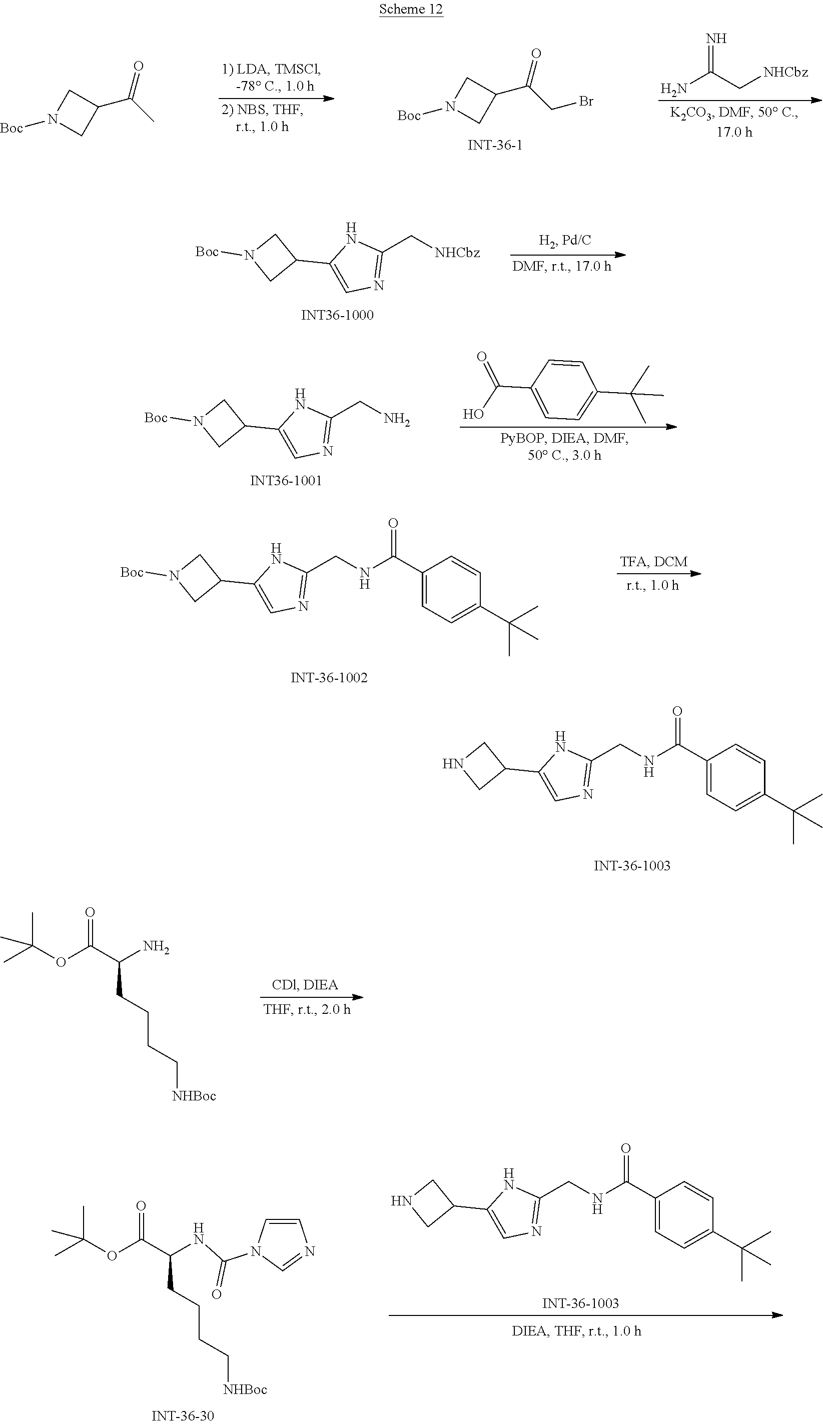

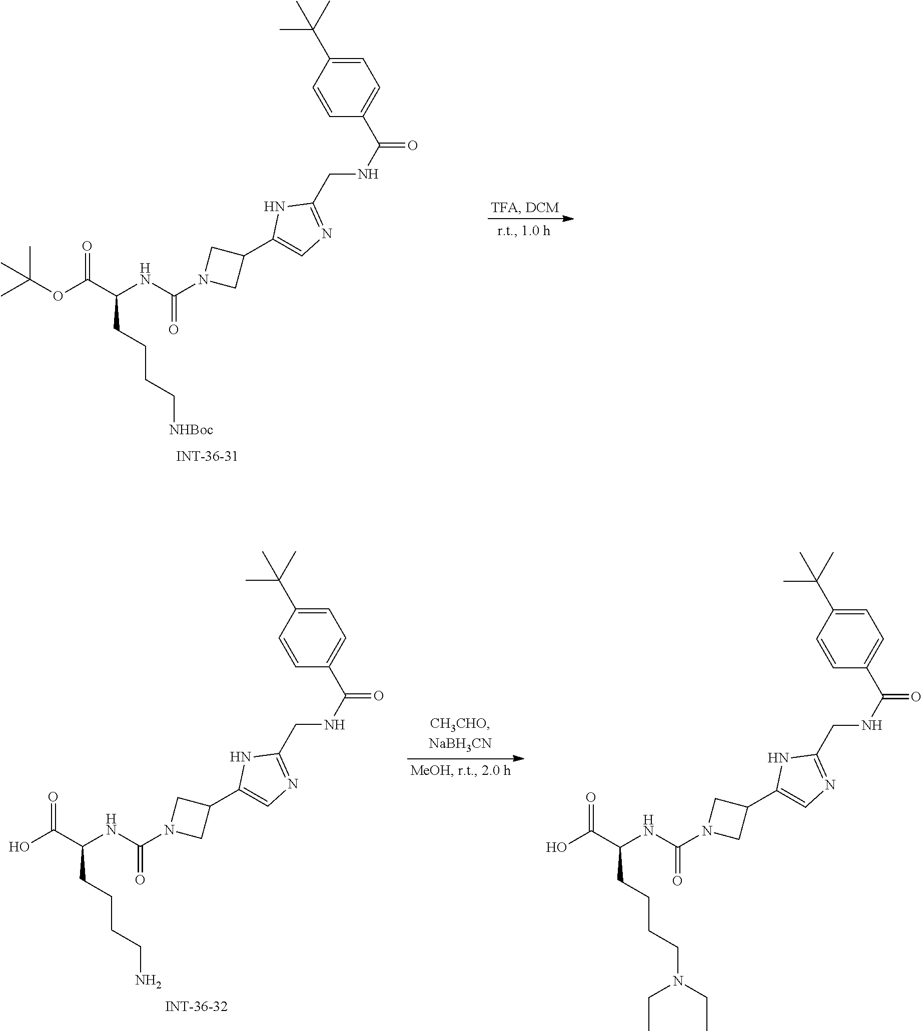 [SMILES: CC(C)(C)OC(=O)NCCCC[C@H](NC(=O)N1CC(c2cnc(CNC(=O)c3ccc(C(C)(C)C)cc3)[nH]2)C1)C(=O)OC(C)(C)C ; CC(C)(C)c1ccc(C(=O)NCc2ncc(C3CN(C(=O)N[C@@H](CCCCN)C(=O)O)C3)[nH]2)cc1 ; CCN(CC)CCCC[C@H](NC(=O)N1CC(c2cnc(CNC(=O)c3ccc(C(C)(C)C)cc3)[nH]2)C1)C(=O)O]

### Step 1: Synthesis of tert-butyl 3-(2-bromoacetyl)azetidine-1-carboxylate

Into a 250 ml flask was added tert-butyl 3-acetylazetidine-1-carboxylate (2.00 g, 10.04 mmol, 1.00 equiv), THF (30.00 mL), the mixture was cooled to −78° C. and under N₂ atmosphere. LDA (in 2M THF) (6.02 mL, 12.05 mmol, 1.20 equiv) was added to the above solution dropwise, the mixture was stirred at −78° C. for 40 mins, then TMSCl (1.97 g, 18.17 mmol, 1.81 equiv) was added dropwise, stirred for another 1.0 h. The NaHCO₃ solution (50 mL) was added, extracted with EA (3×50 mL), washed by NaCl solution (50 mL), dried by Na₂SO₄ (filtered out), the organic phase was concentrated. The crude product was dissolved in THF (30.00 mL), cooled to 0° C., then NaHCO₃ (1.10 g, 13.05 mmol, 1.30 equiv), NBS (1.61 g, 9.03 mmol, 0.90 equiv) was added, the reaction was stirred at r.t. for 1.0 h. The reaction was quenched by the addition of NaHCO₃ solution (20 mL) at room temperature. The resulting mixture was extracted with EtOAc (3×50 mL). The combined organic layers were washed with NaHCO₃ solution (30 mL), NaCl solution (30 mL), dried over anhydrous Na₂SO₄. After filtration, the filtrate was concentrated under reduced pressure. This resulted in tert-butyl 3-(2-bromoacetyl)azetidine-1-carboxylate (4.00 g, crude) as a yellow oil. LC/MS: mass calcd. for C₁₀H₁₆BrNO₃: 277.03, found: 221.95, 223.95 [M−t−Bu+1+H]⁺.

### Step 2: Synthesis of tert-butyl 3-[2-({[(benzyloxy)carbonyl]amino}methyl)-3H-imidazol-4-yl]azetidine-1-carboxylate

To a stirred solution of tert-butyl 3-(2-bromoacetyl)azetidine-1-carboxylate (2.60 g, 9.35 mmol, 1.00 equiv) in DMF (8.00 mL), benzyl N-(carbamimidoylmethyl)carbamate (581.14 mg, 2.80 mmol, 0.30 equiv) and K₂CO₃ (1.68 g, 12.15 mmol, 1.30 equiv) were added and the resulting mixture was stirred for 17.0 h at 50° C. The resulting mixture was filtered, the filter cake was washed with CH₃OH (3×3 mL). The filtrate was concentrated under reduced pressure and the residue was purified by reverse flash chromatography with the following conditions: column, C18 silica gel; mobile phase, ACN in water (0.05% NH4HCO3), 5% to 45% gradient in 40 min; detector, UV 254 nm. The fraction were combined and concentrated to afford tert-butyl 3-[2-({[(benzyloxy)carbonyl]amino}methyl)-3H-imidazol-4-yl]azetidine-1-carboxylate (350.00 mg, 9.69% yield) as a brown oil. LC/MS: mass calcd. for C₂₀H₂₆N₄O₄: 386.19, found: 387.20 [M+H]⁺.

### Step 3: Synthesis of tert-butyl 3-[2-(aminomethyl)-3H-imidazol-4-yl] azetidine-1-carboxylate

To a solution of tert-butyl 3-[2-({[(benzyloxy)carbonyl]amino}methyl)-3H-imidazol-4-yl]azetidine-1-carboxylate (330.00 mg, 0.85 mmol, 1.00 equiv) in DMF (2.00 mL), Pd/C (99.00 mg, 30% w/w) was added and the reaction was stirred for 17.0 h at room temperature under H₂ atmosphere. The resulting mixture was filtered, the filter cake was washed with MeOH (2×1 mL). The crude was purified by reverse flash chromatography with the following conditions: column, C18 silica gel; mobile phase, ACN in water (0.05% NH₄HCO₃), 5% to 30% gradient in 30 min; detector, UV 254 nm. The filtrate was concentrated under reduced pressure to afford tert-butyl 3-[2-(aminomethyl)-3H-imidazol-4-yl]azetidine-1-carboxylate (90.00 mg, 41.77% yield) as a brown oil. LC/MS: mass calcd. for C₁₂H₂₀N₄O₂: 252.15, found: 253.30 [M+H]⁺.

### Step 4: Synthesis of tert-butyl 3-(2-{[(4-tert-butylphenyl)formamido]methyl}-3H-imidazol-4-yl)azetidine-1-carboxylate

The procedure was the same as tert-butyl 2-(2-{2-[(4-tert-butylphenyl) formamido]ethoxy}ethoxy)acetate (INT-29-300), but the reaction temperature was 50° C. and reaction time was 3.0 h. The final product was purified by TLC-Plate. 80.00 mg of tert-butyl 3-[2-(aminomethyl)-3H-imidazol-4-yl]azetidine-1-carboxylate was used, 120.00 mg of desired product was obtained as brown oil (91.97% yield).

### Step 5: Synthesis of N-{[4-(azetidin-3-yl)-3H-imidazol-2-yl]methyl}-4-tert-butylbenzamide

The procedure was the same as methyl (2S)-2-[(2S)-2-[(2S)-2-amino-5-(morpholin-4-yl)pentanamido]-6-{[(9H-fluoren-9-ylmethoxy)carbonyl]amino} hexanamido]-3-hydroxypropanoate (INT-50-6). 100.00 mg of tert-butyl 3-(2-{[(4-tert-butylphenyl)formamido]methyl}-3H-imidazol-4-yl)azetidine-1-carboxylate was used, 100.00 mg crude of desired product was obtained. LC/MS: mass calcd. for C₁₈H₂₄N₄O: 312.19, found: 313.55 [M+H]⁺.

### Step 6: Synthesis of tert-butyl (2S)-6-[(tert-butoxycarbonyl)amino]-2-(imidazole-1-carbonylamino)hexanoate

To a stirred solution of tert-butyl (2S)-2-amino-6-[(tert-butoxycarbonyl) amino]hexanoate (300.00 mg, 0.99 mmol, 1.00 equiv) in THF (2.00 mL), DIEA (384.63 mg, 2.98 mmol, 3.00 equiv) and CDI (804.27 mg, 4.96 mmol, 5.00 equiv) were added and the resulting mixture was stirred for 2.0 h at room temperature. The resulting mixture was poured into water (50 mL), extracted with DCM (2×100 mL). The combined organic layers were washed with water (3×150 mL), dried over anhydrous Na₂SO₄. After filtration, the filtrate was concentrated under reduced pressure to afford tert-butyl (2S)-6-[(tert-butoxycarbonyl)amino]-2-(imidazole-1-carbonylamino)hexanoate (350.00 mg, 88.99% yield) as a colorless oil. LC/MS: mass calcd. for C₁₉H₃₂N₄O₅: 396.23, found: 397.15 [M+H]⁺.

### Step 7: Synthesis of tert-butyl (2S)-6-[(tert-butoxycarbonyl)amino]-2-[3-(2-{[(4-tert-butylphenyl)formamido]methyl}-3H-imidazol-4-yl)azetidine-1-carbonylamino]hexanoate

To a stirred solution of N-{[4-(azetidin-3-yl)-3H-imidazol-2-yl]methyl}-4-tert-butylbenzamide (80.00 mg, 0.26 mmol, 1.00 equiv) in THF (3.00 mL), DIEA (99.29 mg, 0.77 mmol, 3.00 equiv) and N-{[4-(azetidin-3-yl)-3H-imidazol-2-yl] methyl}-4-tert-butylbenzamide (80.00 mg, 0.26 mmol, 1.00 equiv) were added and the resulting mixture was stirred for 1.0 h at room temperature. The resulting mixture was concentrated under reduced pressure and purified by Prep-TLC (DCM:MeOH=18:1) to afford tert-butyl (2S)-6-[(tert-butoxycarbonyl)amino]-2-[3-(2-{[(4-tert-butylphenyl)formamido]methyl}-3H-imidazol-4-yl)azetidine-1-carbonylamino]hexanoate (85.00 mg, 51.80% yield) as a light yellow solid. LC/MS: mass calcd. for C₃₄H₅₂N₆O₆: 640.39, found: 641.70 [M+H]⁺.

### Step 8: Synthesis of (2S)-6-amino-2-[3-(2-{[(4-tert-butylphenyl)formamido] methyl}-3H-imidazol-4-yl)azetidine-1-carbonylamino]hexanoic acid

The procedure was the same as methyl (2S)-2-[(2S)-2-[(2S)-2-amino-5-(morpholin-4-yl)pentanamido]-6-{[(9H-fluoren-9-ylmethoxy)carbonyl]amino} hexanamido]-3-hydroxypropanoate (INT-50-6). 80.00 mg of tert-butyl (2S)-6-[(tert-butoxycarbonyl)amino]-2-[3-(2-{[(4-tert-butylphenyl)formamido]methyl}-3H-imidazol-4-yl)azetidine-1-carbonylamino]hexanoate was used, 80.00 mg crude of desired product was obtained as brown oil. LC/MS: mass calcd. for C₂₅H₃₆N₆O₄: 484.27, found: 485.45 [M+H]⁺.

### Step 9: synthesis of (2S)-2-[3-(2-{[(4-tert-butylphenyl)formamido]methyl}-3H-imidazol-4-yl)azetidine-1-carbonylamino]-6-(diethylamino)hexanoic acid

The procedure was the same as benzyl (2S)-2-[(2S)-6-{bicycle[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-024-OBn). 60.00 mg of (2S)-6-amino-2-[3-(2-{[(4-tert-butylphenyl)formamido]methyl}-3H-imidazol-4-yl)azetidine-1-carbonylamino]hexanoic acid was used, 60.00 mg of desired product was obtained as white solid (89.62% yield). LC/MS: mass calcd. for C₂₉H₄₄N₆O₄: 540.34, found: 541.55 [M+H]⁺.

### Example 13. Synthesis of 2-(3,5-dimethylphenoxy)-1-[4-(3-hydroxy-2-methoxybenzoyl)piperazin-1-yl]ethanone (INT-37)

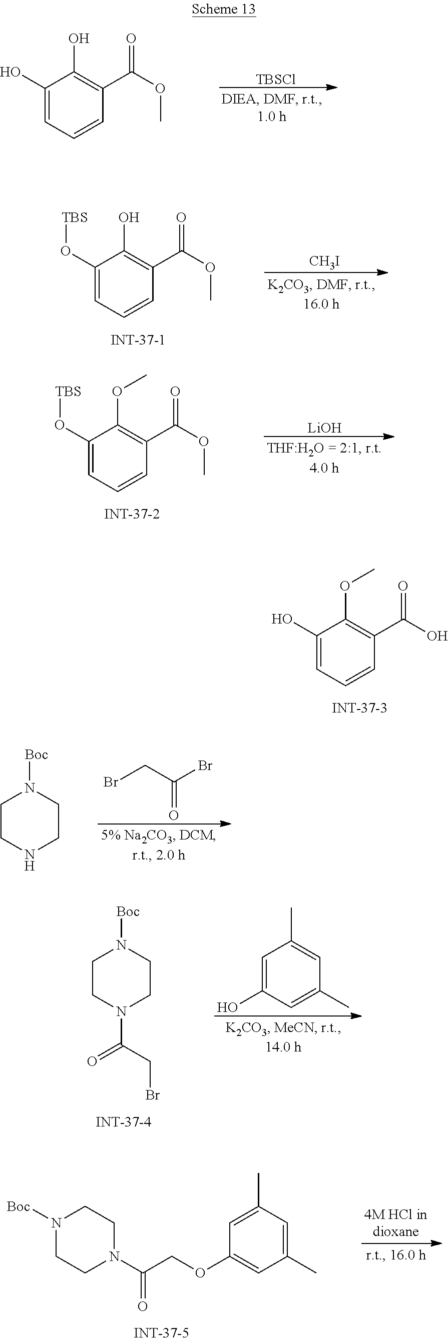

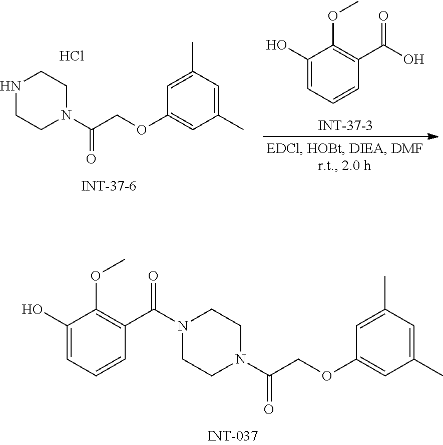 [SMILES: Cc1cc(C)cc(OCC(=O)N2CCNCC2)c1 ; Cl ; COc1c(O)cccc1C(=O)O ; COc1c(O)cccc1C(=O)N1CCN(C(=O)COc2cc(C)cc(C)c2)CC1]

### Step 1: Synthesis of methyl 3-[(tert-butyldimethylsilyl)oxy]-2-hydroxybenzoate

To a stirred solution of methyl 2,3-dihydroxybenzoate (10.00 g, 59.47 mmol, 1.00 equiv) in DMF (300.00 mL) was added DIEA (10.76 g, 83.26 mmol, 1.40 equiv) and t-butyldimethylchlorosilane (9.86 g, 65.42 mmol, 1.10 equiv) dropwise at 0° C. under nitrogen atmosphere. The mixture was stirred at 0° C. for 1.0 h. The mixture was added with water (300 mL) and extracted by EA (3×200 mL). The organic layer was washed with water (300 mL) and brine (150 mL). The organic layer was dried over Na₂SO₄. The mixture was filtered through a Celite pad and concentrated under reduced pressure. It was purified by silica gel column chromatography (PE/EA=10:1). 16.90 g of desired product was obtained as yellow oil (99.00% yield). LC/MS: mass calcd. For C₁₄H₂₂O₄Si: 282.13, found: 283.00 [M+H]⁺.

### Step 2: Synthesis of methyl 3-[(tert-butyldimethylsilyl)oxy]-2-methoxybenzoate

To a stirred mixture of methyl 3-[(tert-butyldimethylsilyl)oxy]-2-hydroxybenzoate (11.40 g, 40.37 mmol, 1.00 equiv) and K₂CO₃ (10.04 g, 72.66 mmol, 1.80 equiv) in DMF (200.00 mL) was added CH₃I (10.31 g, 72.66 mmol, 1.80 equiv) at rt. The mixture was stirred for 16.0 h. The resulting mixture was filtered, the filter cake was washed with EA (200 mL). The organic layer was washed with brine (3×100 mL) and dried over Na₂SO₄. The solvent was concentrated under reduced pressure. 12.00 g of desired product was obtained as light yellow solid (99% yield). LC/MS: mass calcd. For C₁₅H₂₄O₄Si: 296.14, found: 297.15[M+H]⁺.

### Step 3: Synthesis of 3-hydroxy-2-methoxybenzoic acid

To a stirred mixture of methyl 3-[(tert-butyldimethylsilyl)oxy]-2-methoxybenzoate (12.00 g, 40.48 mmol, 1.00 equiv) in H₂O (40.00 mL) and THF (80.00 mL) was added LiOH (14.54 g, 607.22 mmol, 15.00 equiv) at room temperature. The mixture was stirred at r.t. for 4.0 h. The mixture was concentrated under reduced pressure. The mixture was added with water and washed with EA. The aqueous layer was acidified with 3 M HCl to pH=1. The mixture was extracted by EA and the organic layer was dried over Na₂SO₄. The solvent was concentrated under reduced pressure. 5.60 g of desired product was obtained as yellow oil (82.27% yield). LC/MS: mass calcd. For C₈H₈O₄: 168.04, found: 169.05[M+H]⁺.

### Step 4: Synthesis of tert-butyl 4-(2-bromoacetyl)piperazine-1-carboxylate

A solution of tert-butyl piperazine-1-carboxylate (5.60 g, 30.06 mmol, 1.41 equiv) in Na₂CO₃ (10.00 mL, 238.70 mmol, 11.20 equiv) was treated with DCM (100.00 mL). To the mixture was added bromoacetyl bromide (4.30 g, 21.30 mmol, 1.00 equiv) in portions for 40 min at 0° C. Then the resulting mixture was stirred for 2.0 h at room temperature. The reaction was quenched by water and was extracted with EtOAc (3×30 mL). The combined organic layers were washed with 5% NaHCO₃ (3×30 mL) and aq. citric acid (3×30 mL), dried over Na₂SO₄. After filtration, the filtrate was concentrated under reduced pressure. 7.40 g of desired product was obtained as white solid (97.25% yield). For C₁₁H₁₉BrN₂O₃: 306.06, 308.06 found: 251.00, 253.00[M−Boc+H+H]⁺.

### Step 5: Synthesis of tert-butyl 4-[2-(3,5-dimethylphenoxy) acetyl]piperazine-1-carboxylate

A mixture of tert-butyl 4-(2-bromoacetyl)piperazine-1-carboxylate (1.00 g, 3.26 mmol, 1.00 equiv), 3,5-dimethylphenol (0.60 g, 4.88 mmol, 1.50 equiv) and K₂CO₃ (1.35 g, 9.77 mmol, 3.00 equiv) in CH₃CN (20.00 mL) was stirred for 14.0 h at rt. The reaction was quenched by water and was extracted with EA (3×200 mL). The organic layers were combined, dried over Na₂SO₄, filtered and concentrated.

The residue obtained was purified by silica gel chromatography (EA/PE=1:3). The fractions were combined and concentrated. 1.00 g of desired product was obtained as light yellow oil (88.16% yield). LC/MS: mass calcd. For C₁₉H₂₈N₂O₄: 348.44, found: 349.20 [M+H]⁺.

### Step 6: Synthesis of 2-(3,5-dimethylphenoxy)-1-(piperazin-1-yl)ethanone

The procedure was the same as methyl (2S)-2-[(2S)-2-amino-6-{[(9H-fluoren-9-yl)methoxy)carbonyl]amino}hexanamido]-3-hydroxypropanoate (INT-14.6), but after the reaction, the mixture was concentrated. The crude was used for next step directly. 1.00 g of tert-butyl 4-[2-(3,5-dimethylphenoxy)acetyl]piperazine-1-carboxylate was used. 0.70 g of desired product was obtained as off-white solid (98.22% yield). LC/MS: mass calcd. For C₁₄H₂₀N₂O₂: 248.33, found: 249.20 [M+H]⁺.

### Step 7: Synthesis of 2-(3,5-dimethylphenoxy)-1-[4-(3-hydroxy-2-methoxybenzoyl)piperazin-1-yl]ethanone

A mixture of 2-(3,5-dimethylphenoxy)-1-(piperazin-1-yl)ethanone (324.90 mg, 1.31 mmol, 1.10 equiv), EDCI (342.02 mg, 1.78 mmol, 1.50 equiv), HOBT (241.07 mg, 1.78 mmol, 1.50 equiv) and DIEA (384.30 mg, 2.97 mmol, 2.50 equiv) in DMF (5.00 mL) was stirred for 2.0 h at room temperature. To the mixture was added H₂O (5 mL) and extracted by EtOAc (3×10 mL). The organic layer was combined, washed by sat. aq. citric acid (2×30 mL) and sat. aq. NaHCO₃ (2×30 mL) and brine (30 mL), dried over Na₂SO₄, filtered out and concentrated under reduced pressure. The residue was purified by silica gel chromatography (EA/PE=1:1). The fractions were combined and concentrated. 0.28 g of desired product was obtained as white solid (57.10% yield). LC/MS: mass calcd. For C₂₂H₂₆N₂O₅: 398.46, found: 399.20 [M+H]⁺.

### Example 14. Synthesis of 4-{2-[4-(2,3-dimethoxybenzoyl)piperazin-1-yl]-2-oxoethoxy}-2-methylphenylurea (SM-045(OMe)-pUA)

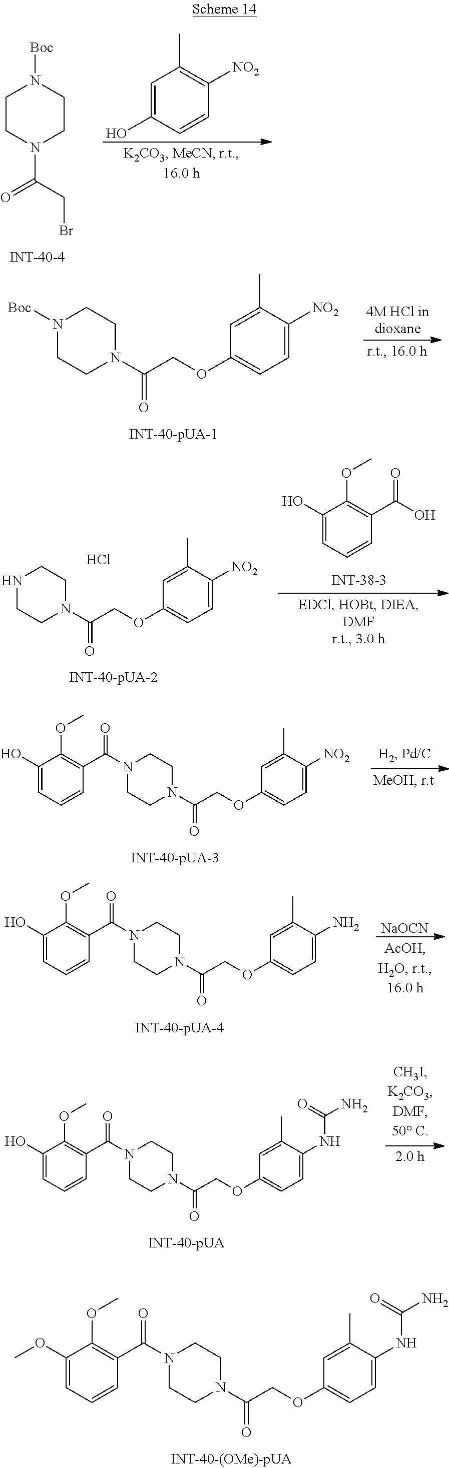

### Step 1: Synthesis of tert-butyl 4-[2-(3-methyl-4-nitrophenoxy)acetyl] piperazine-1-carboxylate

The procedure was the same as tert-butyl 4-[2-(3,5-dimethylphenoxy) acetyl]piperazine-1-carboxylate (INT-37-5). 1.00 g of tert-butyl 4-(2-bromoacetyl)piperazine-1-carboxylate was used. 1.20 g of desired product was obtained as light yellow oil (97.16% yield). LC/MS: mass calcd. For C₁₈H₂₅N₃O₆: 379.17, found: 379.97 [M+H]⁺.

### Step 2: Synthesis of 2-(3-methyl-4-nitrophenoxy)-1-(piperazin-1-yl)ethanone

The procedure was the same as methyl (2S)-2-[(2S)-2-amino-6-{[(9H-fluoren-9-yl)methoxy)carbonyl]amino}hexanamido]-3-hydroxypropanoate (INT-14.6), but the reaction was 16.0 h, the mixture was concentrated. The crude was used for next step directly. 1.20 g of tert-butyl 4-[2-(3-methyl-4-nitrophenoxy)acetyl] piperazine-1-carboxylate was used. 1.00 g crude of desired product was obtained as white solid. LC/MS: mass calcd. For C₁₃H₁₇N₃O₄: 279.12, found: 279.90 [M+H]⁺.

### Step 3: Synthesis of 1-[4-(3-hydroxy-2-methoxybenzoyl)piperazin-1-yl]-2-(3-methyl-4-nitrophenoxy)ethanone

The procedure was the same as 2-(3,5-dimethylphenoxy)-1-[4-(3-hydroxy-2-methoxybenzoyl)piperazin-1-yl]ethanone (INT-037). 0.59 g of 2-(3-methyl-4-nitrophenoxy)-1-(piperazin-1-yl)ethanone was used. 0.36 g of desired product was obtained as light yellow oil (46.99% yield). LC/MS: mass calcd. For C₂₁H₂₃N₃O₇: 429.15, found: 429.95 [M+H]⁺.

### Step 4: Synthesis of 2-(4-amino-3-methylphenoxy)-1-[4-(3-hydroxy-2-methoxybenzoyl)piperazin-1-yl]ethanone

The procedure was the same as 2-(4-amino-3,5-dimethylphenoxy)-1-[4-(3-hydroxy-2-methoxybenzoyl)piperazin-1-yl]ethanone (INT-38-pNH₂), but the reaction time is 16.00 h. 0.36 g of 1-[4-(3-hydroxy-2-methoxybenzoyl) piperazin-1-yl]-2-(3-methyl-4-nitrophenoxy)ethanone was used. 0.35 g of desired product was obtained as light yellow oil (94.07% yield). LC/MS: mass calcd. For C₂₁H₂₅N₃O₅: 399.18, found: 399.90 [M+H]⁺.

### Step 5: Synthesis of 4-{2-[4-(3-hydroxy-2-methoxybenzoyl)piperazin-1-yl]-2-oxoethoxy}-2-methylphenylurea

The procedure was the same as 4-{2-[4-(3-hydroxy-2-methoxybenzoyl) piperazin-1-yl]-2-oxoethoxy}-2,6-dimethylphenylurea (INT-38-pUA). 0.30 g of 2-(4-amino-3-methylphenoxy)-1-[4-(3-hydroxy-2-methoxybenzoyl)piperazin-1-yl]ethanone was used. 0.13 g of desired product was obtained ad white solid (38.98% yield). LC/MS: mass calcd. For C₂₃H₂₈N₄O₆: 456.20, found: 457.20 [M+H]⁺.

### Step 6: Synthesis of 4-{2-[4-(2,3-dimethoxybenzoyl)piperazin-1-yl]-2-oxoethoxy}-2-methylphenylurea

To a stirred mixture of 4-{2-[4-(3-hydroxy-2-methoxybenzoyl)piperazin-1-yl]-2-oxoethoxy}-2-methylphenylurea (15.00 mg, 0.03 mmol, 1.00 equiv) and K₂CO₃ (8.43 mg, 0.06 mmol, 1.80 equiv) in DMF (1.00 mL) was added CH₃I (8.66 mg, 0.06 mmol, 1.8 equiv) at RT. The resulting mixture was stirred for 2 h at 50° C. The resulting mixture was filtered, the filtrate was then purified by Prep-HPLC with the following conditions: Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 22% B to 24% B in 10 min, 24% B; Wave Length: 254 nm; RT1 (min): 8.92; Number Of Runs: 5. The fractions were combined and concentrated. 2.00 mg of desired product was obtained as off-white solid (12.92% yield). LC/MS: mass calcd. For C₂₃H₂₈N₄O₆: 456.20, found: 457.20 [M+H]⁺.

### Example 15. Synthesis of 2-[(4-tert-butylphenyl)formamido]-N-[2-(3-{2-[4-(2,3-dimethoxybenzoyl)piperazin-1-yl]-2-oxoethoxy}phenoxy)ethyl]acetamide (INT-041)

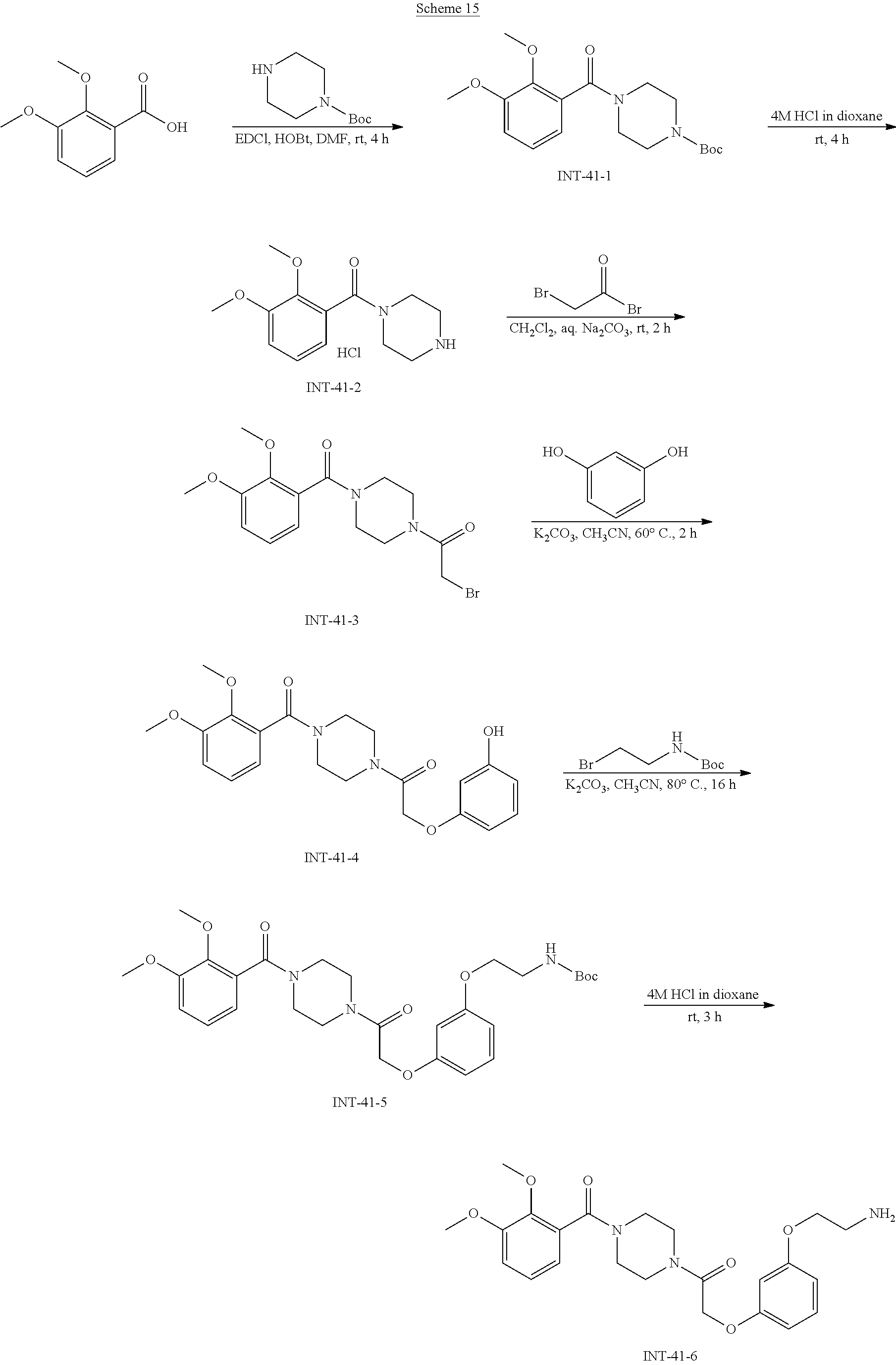

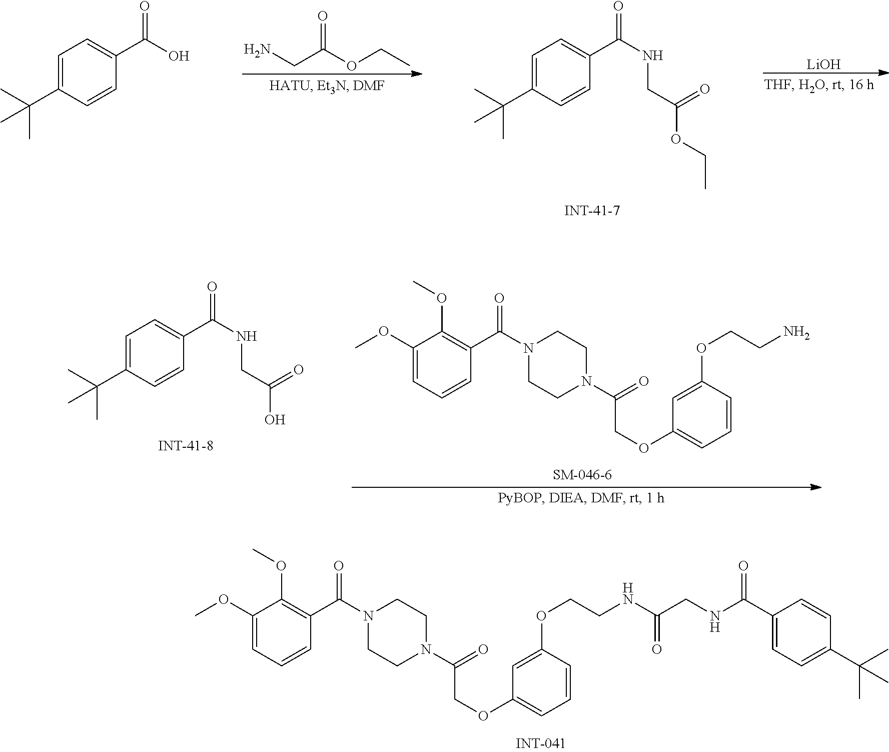

### Step 1: Synthesis of tert-butyl 4-(2,3-dimethoxybenzoyl)piperazine-1-carboxylate

The procedure was the same as 2-(3,5-dimethylphenoxy)-1-[4-(3-hydroxy-2-methoxybenzoyl)piperazin-1-yl]ethanone (INT-037), but the reaction time is 4.0 h. 2.00 g of 2,3-dimethoxybenzoic acid was used. 4.20 g crude of desired product was obtained as white solid. LC/MS: mass calcd. For C₁₈H₂₆N₂O₅: 350.18, found: 351.10 [M+H]⁺.

### Step 2: Synthesis of 1-(2,3-dimethoxybenzoyl)piperazine

The procedure was the same as methyl (2S)-2-[(2S)-2-amino-6-{[(9H-fluoren-9-yl)methoxy)carbonyl]amino}hexanamido]-3-hydroxypropanoate (INT-14.6), but the reaction is 4.0 h, the mixture was concentrated. The crude was used for next step directly. 4.20 g of tert-butyl 4-(2,3-dimethoxybenzoyl)piperazine-1-carboxylate was used. 2.90 g crude of desired product was obtained as white solid. LC/MS: mass calcd. For C₁₃H₁₈N₂O₃: 250.13, found: 251.05 [M+H]⁺.

### Step 3: Synthesis of 2-bromo-1-[4-(2,3-dimethoxybenzoyl)piperazin-1-yl] ethanone

To a stirred solution of 1-(2,3-dimethoxybenzoyl)piperazine (2.60 g, 10.39 mmol, 1.00 equiv) in CH₂Cl₂ (5%, 50 mL) and aq. Na₂CO₃ (5%, 50 mL) was added bromoacetyl bromide (2.31 g, 11.43 mmol, 1.10 equiv) dropwise at 0° C. The resulting mixture was stirred for 2.0 h at room temperature. The resulting mixture was diluted with DCM (10 mL). The resulting mixture was washed with 2×10 mL of 5% HCl. The combined organic layers were washed with brine, H₂O, dried over anhydrous Na₂SO₄. After filtration, the filtrate was concentrated under reduced pressure. 3.50 g of desired product was obtained as yellow solid (90.76% yield). LC/MS: mass calcd. For C₁₅H₁₉BrN₂O₄: 370.05, 372.05, found: 371.00, 373.00 [M+H]⁺.

### Step 4: Synthesis of 1-[4-(2,3-dimethoxybenzoyl)piperazin-1-yl]-2-(3-hydroxyphenoxy)ethanone

The procedure was the same as tert-butyl 4-[2-(3,5-dimethylphenoxy)acetyl]piperazine-1-carboxylate (INT-37-5), but the reaction temperature was 60° C., the reaction time was 2.0 h. 3.20 g of 2-bromo-1-[4-(2,3-dimethoxybenzoyl)piperazin-1-yl]ethanone was used. 2.4 g of desired product was obtained as light yellow oil (69.53% yield). LC/MS: mass calcd. For C₂₁H₂₄N₂O₆: 400.16, found: 401.25 [M+H]⁺.

### Step 5: Synthesis of tert-butyl N-[2-(3-{2-[4-(2,3-dimethoxybenzoyl) piperazin-1-yl]-2-oxoethoxy}phenoxy)ethyl]carbamate

The procedure was the same as tert-butyl 4-[2-(3,5-dimethylphenoxy) acetyl]piperazine-1-carboxylate (INT-37-5), but the reaction temperature was 80° C., the reaction time was 2.0 h. 0.20 g of 1-[4-(2,3-dimethoxybenzoyl)piperazin-1-yl]-2-(3-hydroxyphenoxy)ethanone was used. 0.25 g of desired product was obtained as yellow solid (92.08% yield). LC/MS: mass calcd. For C₂₈H₃₇N₃O₈: 543.26, found: 544.30 [M+H]⁺.

### Step 6: Synthesis of 2-[3-(2-aminoethoxy)phenoxy]-1-[4-(2,3-dimethoxybenzoyl)piperazin-1-yl]ethanone

The procedure was the same as methyl (2S)-2-[(2S)-2-amino-6-{[(9H-fluoren-9-yl)methoxy)carbonyl]amino}hexanamido]-3-hydroxypropanoate (INT-14.6), but the reaction is 3.0 h, the mixture was concentrated. The crude was used for next step directly. 0.33 g of tert-butyl N-[2-(3-{2-[4-(2,3-dimethoxybenzoyl)piperazin-1-yl]-2-oxoethoxy}phenoxy)ethyl]carbamate was used. 0.24 g crude of desired product was obtained as yellow solid. LC/MS: mass calcd. For C₂₃H₂₉N₃O₆: 443.21, found: 444.15 [M+H]⁺.

### Step 7: Synthesis of ethyl 2-[(4-tert-butylphenyl)formamido]acetate

To a stirred solution of 4-tert-butylbenzoic acid (500.00 mg, 2.81 mmol, 1.00 equiv) and amino-acetic acid ethyl ester (347.15 mg, 3.37 mmol, 1.20 equiv) in DMF (10.00 mL) were added HATU (1.60 g, 4.21 mmol, 1.50 equiv) and Et₃N (567.75 mg, 5.61 mmol, 2.00 equiv). The resulting mixture was stirred for 2.0 h at room temperature. The resulting mixture was diluted with water (20 mL). The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (5:1) to afford ethyl 2-[(4-tert-butylphenyl)formamido]acetate (700.00 mg, 94.75%) as a white solid. LC/MS: mass calcd. For C₁₅H₂₁NO₃: 263.15, found: 264.10 [M+H]⁺.

### Step 8: Synthesis of [(4-tert-butylphenyl)formamido]acetic acid

The procedure was the same as (2S)-2-[(4-tert-butylphenyl) formamido]-3-phenylpropanoic acid (INT-14.2), but the reaction time was 16.0 h. 0.70 g of ethyl 2-[(4-tert-butylphenyl)formamido]acetate was used. 0.50 g of desired product was obtained as white solid (79.95% yield). LC/MS: mass calcd. For C₁₃H₁₇NO₃: 235.12, found: 236.10 [M+H]⁺.

### Step 9: Synthesis of 2-[(4-tert-butylphenyl)formamido]-N-[2-(3-{2-[4-(2,3-dimethoxybenzoyl)piperazin-1-yl]-2-oxoethoxy}phenoxy)ethyl]acetamide

The procedure was the same as tert-butyl 2-(2-{2-[(4-tert-butylphenyl) formamido]ethoxy}ethoxy)acetate (INT-29-300). After the reaction, the reaction mixture was purified by HPLC. 0.10 g of (4-tert-butylphenyl)formamido] acetic acid was used. 39.80 mg of desired product was obtained as white solid (14.14% yield). LC/MS: mass calcd. For C₃₆H₄₄N₄O₈: 660.32, found: 661.25 [M+H]⁺.

### Example 16. Synthesis of Methyl (2S)-2-[(2S)-2-[(2S)-2-[(2S)-2-{[1-(4-tert-butylphenyl)pyrazol-4-yl] formamido}propanamido]-4-methylpentanamido]-6-(diethylamino)hexanamido]-3-hydroxypropanoate (INT-042)

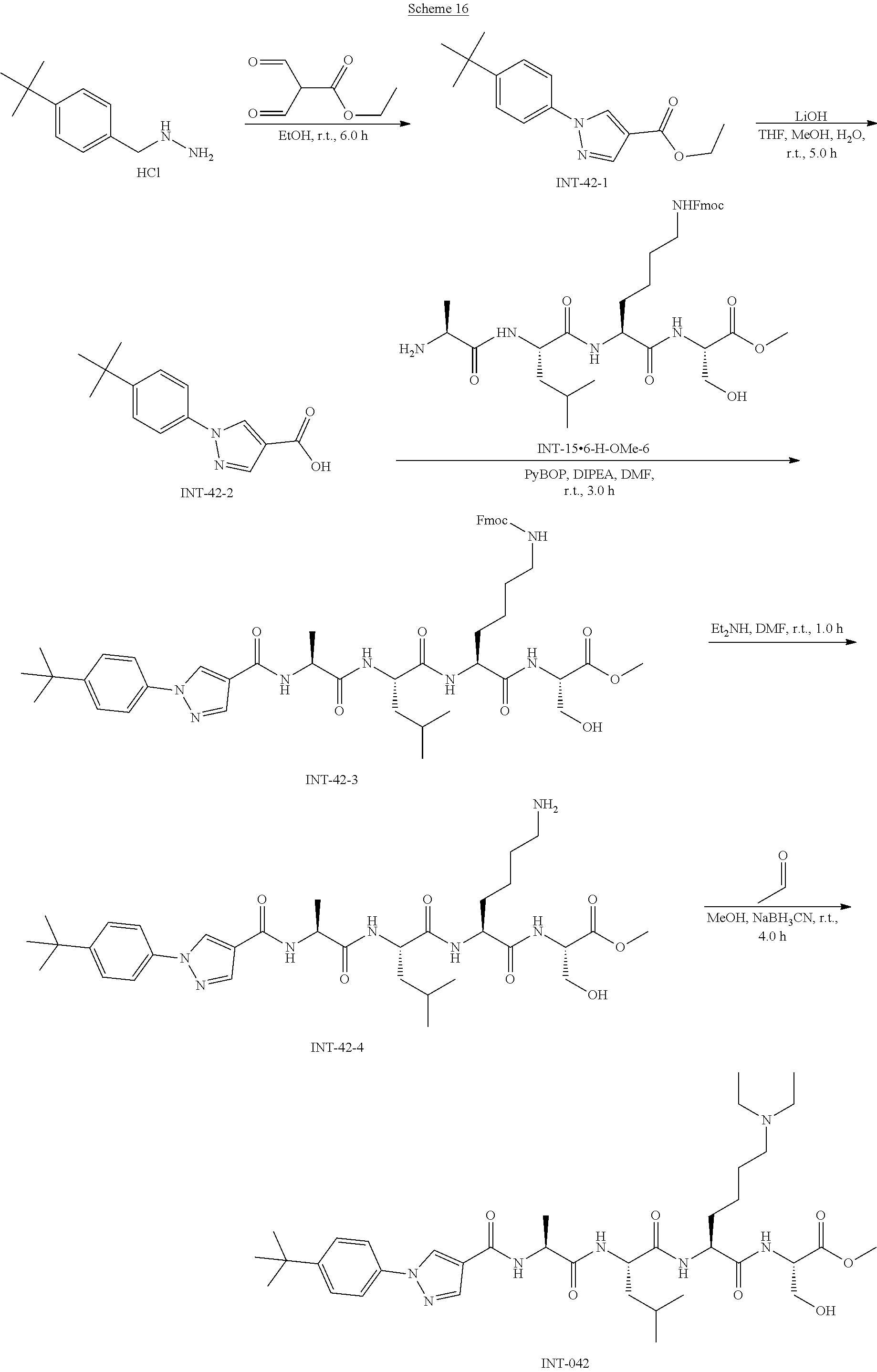

### Step 1: Synthesis of ethyl 1-(4-tert-butylphenyl)pyrazole-4-carboxylate

To a stirred mixture of [(4-tert-butylphenyl)methyl]hydrazine (3.71 g, 20.82 mmol, 1.00 equiv) in ethyl alcohol (120.00 mL) were added ethyl 2-formyl-3-oxopropanoate (3.00 g, 20.82 mmol, 1.00 equiv) dropwise at 0° C. under air atmosphere. The mixture was stirred for 6.0 h at 25° C. The resulting mixture was concentrated under reduced pressure to afford ethyl 1-(4-tert-butylphenyl)pyrazole-4-carboxylate (2.00 g, 35.28%) as a brown solid. LC/MS: mass calcd. For C₁₆H₂₀N₂O₂: 272.15, found: 273.15 [M+H]⁺.

### Step 2: Synthesis of 1-(4-tert-butylphenyl)pyrazole-4-carboxylic acid

The procedure was the same as (2S)-2-[(4-tert-butylphenyl) formamido]-3-phenylpropanoic acid (INT-14.2), but the reaction time is 5.0 h. 1.00 g of ethyl 1-(4-tert-butylphenyl)pyrazole-4-carboxylate was used. 0.64 g of desired product was obtained as white solid (71.19% yield). LC/MS: mass calcd. For C₁₄H₁₆N₂O₂: 244.29, found: 246.10 [M+H]⁺.

### Step 3: Synthesis of methyl (2S)-2-[(2S)-2-[(2S)-2-[(2S)-2-{[1-(4-tert-butylphenyl)pyrazol-4-yl]formamido}propanamido]-4-methylpentanamido]-6-{[(9H-fluoren-9-ylmethoxy)carbonyl]amino}hexanamido]-3-hydroxypropanoate

The procedure was the same as methyl (2S)-2-[(2S)-2-[2-(2-{2-[(4-tert-butylphenyl)formamido]acetamido}acetamido)acetamido]-6-{[(9H-fluoren-9-ylmethoxy)carbonyl]amino}hexanamido]-3-hydroxypropanoate (INT-32-102). 0.26 g of 1-(4-tert-butylphenyl)pyrazole-4-carboxylic acid was used. 0.80 g of desired product was obtained as yellow solid (85.41% yield). LC/MS: mass calcd. For C₄₈H₆₁N₇O₉: 879.45, found: 880.40 [M+H]⁺.

### Step 4: Synthesis of methyl (2S)-2-[(2S)-6-amino-2-[(2S)-2-[(2S)-2-{[1-(4-tert-butylphenyl)pyrazol-4-yl]formamido}propanamido]-4-methylpentanamido]hexanamido]-3-hydroxypropanoate

The procedure was the same as methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-14.11), but the reaction time is 1.0 h. 1.0 g of methyl (2S)-2-[(2S)-2-[(2S)-2-[(2S)-2-{[1-(4-tert-butylphenyl)pyrazol-4-yl]formamido}propanamido]-4-methylpentanamido]-6-{[(9H-fluoren-9-ylmethoxy)carbonyl]amino}hexanamido]-3-hydroxypropanoate was used. 1.0 g crude of desired product was obtained as yellow oil. LC/MS: mass calcd. For C₃₃H₅₁N₇O₇: 657.81, found: 658.60 [M+H]⁺.

### Step 5: Synthesis of methyl (2S)-2-[(2S)-2-[(2S)-2-[(2S)-2-{[1-(4-tert-butylphenyl) pyrazol-4-yl]formamido}propanamido]-4-methylpentanamido]-6-(diethylamino)hexanamido]-3-hydroxypropanoate

The procedure was the same as methyl (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoate (INT-14-OMe), but the reaction time is 4.0 h. 1.0 g of methyl (2S)-2-[(2S)-6-amino-2-[(2S)-2-[(2S)-2-{[1-(4-tert-butylphenyl)pyrazol-4-yl]formamido}propanamido]-4-methylpentanamido]hexanamido]-3-hydroxypropanoate was used. 0.40 g of desired product was obtained as yellow solid (36.86% yield). LC/MS: mass calcd. For C₃₇H₅₉N₇O₇: 713.92, found: 714.45 [M+H]⁺.

### Example 17. Synthesis of (6-{[(4-tert-butylphenyl)methyl]carbamoyl}-3H-imidazo[4,5-b]pyridin-2-yl)acetic acid (INT-043)

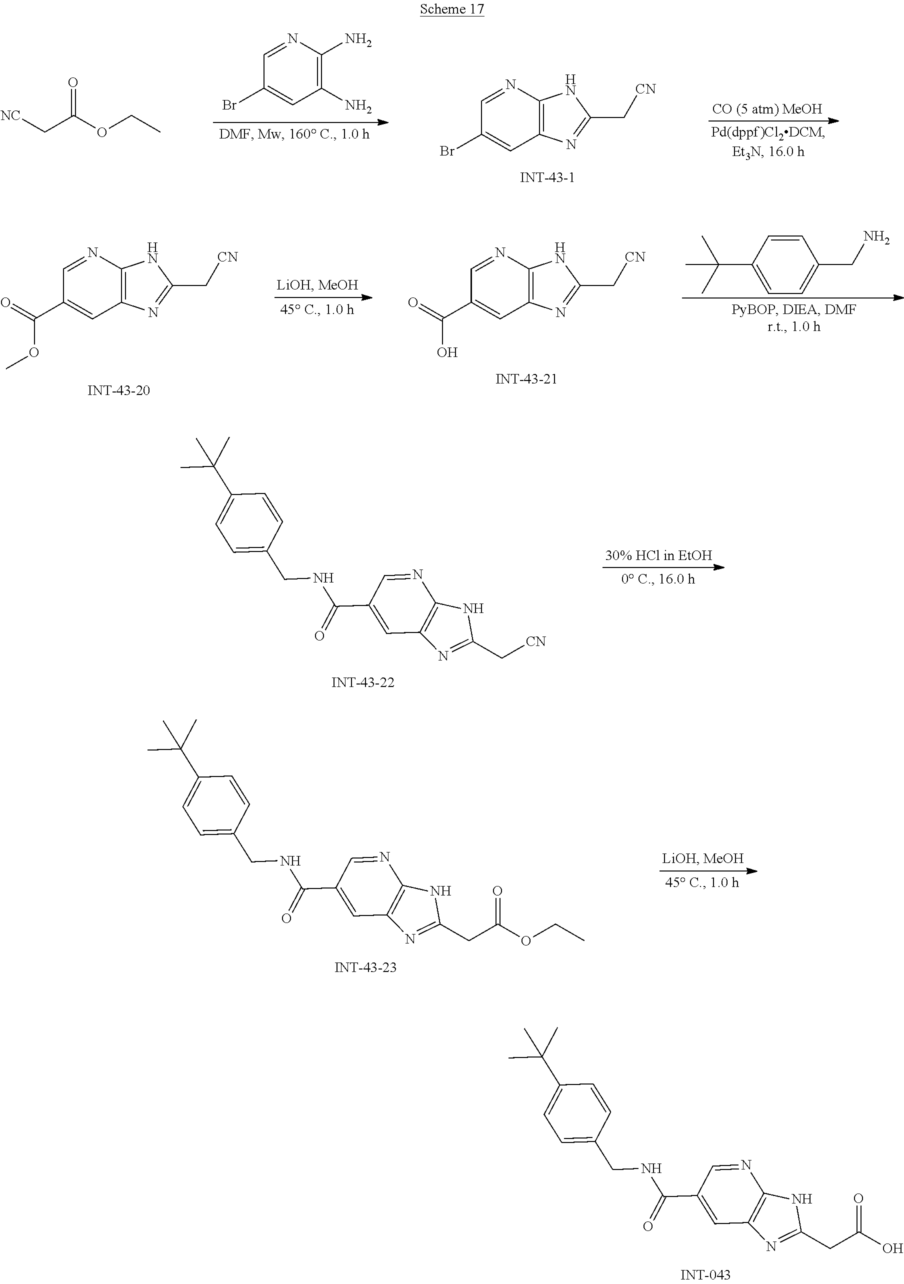

### Step 1: Synthesis of 2-{6-bromo-3H-imidazo[4,5-b]pyridin-2-yl}acetonitrile

A mixture of ethyl cyanoacetate (2.71 g, 23.93 mmol, 1.50 equiv) and 5-bromopyridine-2,3-diamine (3.00 g, 15.96 mmol, 1.00 equiv) in DMF (10.00 mL) was stirred for 1.0 h at 160° C. under microwave condition. The reaction was quenched with water (20 mL) at room temperature. The precipitated solids were collected by filtration and washed with water (3×20 mL). The resulting solid was dried under vacuum to afford 2-{6-bromo-3H-imidazo[4,5-b]pyridin-2-yl}acetonitrile (2.40 g, 63.45% yield) as a brown solid. LC/MS: mass calcd. For C₈H₅BrN₄:235.96, found: 237.00, 239.00 [M+H, M+2+H]⁺.

### Step 2: Synthesis of methyl 2-(cyanomethyl)-3H-imidazo[4,5-b]pyridine-6-carboxylate

To a stirred solution of 2-{6-bromo-3H-imidazo[4,5-b]pyridin-2-yl}acetonitrile (1.80 g, 7.59 mmol, 1.00 equiv) in MeOH (20.00 mL) was added Pd(dppf)Cl₂·CH₂Cl₂ (1.24 g, 1.52 mmol, 0.20 equiv) and Et₃N (2.31 g, 22.78 mmol, 3.00 equiv). The resulting mixture was stirred at 100° C. for 16.0 h under CO atmosphere at 5 bar. The resulting mixture was concentrated under vacuum. The residue was purified by reverse flash chromatography with the following conditions: column, silica gel; mobile phase, MeCN in water (0.05% TFA), 10% to 30% gradient in 30 min; detector, UV 254 n. The fractions were combined and concentrated to afford methyl 2-(cyanomethyl)-3H-imidazo[4,5-b]pyridine-6-carboxylate (510.00 mg, 31.07% yield) as a white solid. LC/MS: mass calcd. For C₁₀H₈N₄O₂: 216.06, found: 217.10 [M+H]⁺.

### Step 3: Synthesis of 2-(cyanomethyl)-3H-imidazo[4,5-b]pyridine-6-carboxylic acid

The procedure was the same as (2S)-2-[(4-tert-butylphenyl) formamido]-3-phenylpropanoic acid (INT-14.2), but the solvent was MeOH and the reaction temperature was 45° C. 490.00 mg of methyl 2-(cyanomethyl)-3H-imidazo[4,5-b]pyridine-6-carboxylate was used, 410.00 mg of desired product was obtained as yellow solid (89.48% yield). LC/MS: mass calcd. For C₉H₆N₄O₂: 202.04, found: 203.00 [M+H]⁺.

### Step 4: Synthesis of N-[(4-tert-butylphenyl)methyl]-2-(cyanomethyl)-3H-imidazo[4,5-b]pyridine-6-carboxamide

The procedure was the same as tert-butyl 2-(2-{2-[(4-tert-butylphenyl) formamido]ethoxy}ethoxy)acetate (INT-29-300). 390.00 mg of 2-(cyanomethyl)-3H-imidazo[4,5-b]pyridine-6-carboxylic acid was used, 660.00 mg of desired product was obtained as yellow solid (98.48% yield). LC/MS: mass calcd. For C₂₀H₂₁N₅O: 347.17, found: 348.25 [M+H]⁺.

### Step 5: Synthesis of ethyl 2-(6-{[(4-tert-butylphenyl)methyl]carbamoyl}-3H-imidazo[4,5-b]pyridin-2-yl)acetate

A solution of N-[(4-tert-butylphenyl)methyl]-2-(cyanomethyl)-3H-imidazo [4,5-b]pyridine-6-carboxamide (660.00 mg, 1.90 mmol, 1.00 equiv) in hydrochloric acid (in ethanol) (30%, 15.00 mL) was stayed at 0° C. for 16.0 h. The resulting mixture was concentrated to afford ethyl 2-(6-{[(4-tert-butylphenyl)methyl] carbamoyl}-3H-imidazo[4,5-b]pyridin-2-yl)acetate (740.00 mg crude) as a yellow solid. LC/MS: mass calcd. For C₂₂H₂₆N₄O₃: 394.20, found: 395.25 [M+H]⁺.

### Step 6: Synthesis of (6-{[(4-tert-butylphenyl)methyl]carbamoyl}-3H-imidazo [4,5-b]pyridin-2-yl)acetic acid

The procedure was the same as (2S)-2-[(2S)-6-(diethylamino)-2-acetamidohexanamido]-3-hydroxypropanoic acid (INT-27-Ac). 200.00 mg of ethyl 2-(6-{[(4-tert-butylphenyl)methyl]carbamoyl}-3H-imidazo[4,5-b]pyridin-2-yl)acetate was used, 185.00 mg crude of desired product was obtained as yellow solid. LC/MS: mass calcd. For C₂₀H₂₂N₄O₃: 366.16, found: 367.35 [M+H]⁺.

### Example 18. Synthesis of tert-butyl N-[2-(4-aminobenzamido)phenyl]carbamate (INT-44 Fragment)

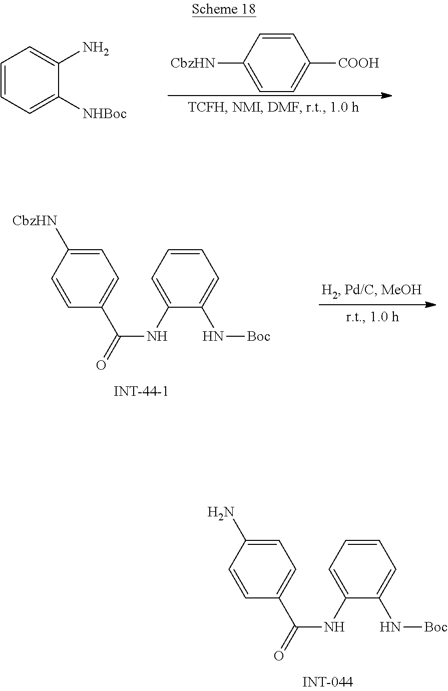 [SMILES: CC(C)(C)OC(=O)Nc1ccccc1N ; O=C(Nc1ccc(C(=O)O)cc1)OCc1ccccc1 ; CC(C)(C)OC(=O)Nc1ccccc1NC(=O)c1ccc(NC(=O)OCc2ccccc2)cc1 ; CC(C)(C)OC(=O)Nc1ccccc1NC(=O)c1ccc(N)cc1]

### Step 44-1: Synthesis of benzyl N-[4-({2-[(tert-butoxycarbonyl)amino]phenyl}carbamoyl)phenyl]carbamate

A solution of tert-butyl N-(2-aminophenyl)carbamate (765.00 mg, 3.67 mmol, 1.00 equiv) and 4-{[(benzyloxy)carbonyl]amino}benzoic acid (1.00 g, 3.69 mmol, 1.00 equiv), TCFH (1.30 g, 4.63 mmol, 1.26 equiv), NMI (1.80 g, 21.92 mmol, 5.95 equiv) in DMF (5.00 mL) was stirred for 1.0 h at room temperature. The reaction was poured into ice water (50 mL), and the mixture was stirred for 15 min. The precipitated solids were collected by filtration and washed with water (3×50 mL) and dried under vacuum.

The residue was purified by silica gel column chromatography, eluted with CH₂Cl₂/MeOH (10:1) to afford benzyl N-[4-({2-[(tert-butoxycarbonyl)amino]phenyl}carbamoyl)phenyl]carbamate (1.40 g, 79.82% yield) as a white solid. LC/MS: mass calcd. for C₂₆H₂₇N₃O₅: 461.20, found: 484.30[M+Na]⁺.

### Step 44-2: Synthesis of tert-butyl N-[2-(4-aminobenzamido)phenyl]carbamate

To a stirred mixture of benzyl N-[4-({2-[(tert-butoxycarbonyl)amino]phenyl}carbamoyl)phenyl]carbamate (1.40 g, 3.03 mmol, 1.00 equiv) in MeOH (20.00 mL) was added Pd/C (280.00 mg, 20% w/w) in portions at room temperature. The resulting mixture was stirred for 1.0 h at room temperature under hydrogen atmosphere. The resulting mixture was filtered, the filter cake was washed with MeOH (3×10 mL). The filtrate was concentrated under reduced pressure. This resulted in tert-butyl N-[2-(4-aminobenzamido)phenyl]carbamate (950.00 mg, crude) as a white solid. LC/MS: mass calcd. for C₁₈H₂₁N₃O₃: 327.16, found: 350.20[M+Na]⁺.

### Example 19. Methyl 4-{[butyl({[4-(prop-2-yn-1-yloxy)phenyl]carbamoyl})amino]methyl}benzoate (INT-046-AR-Fragment)

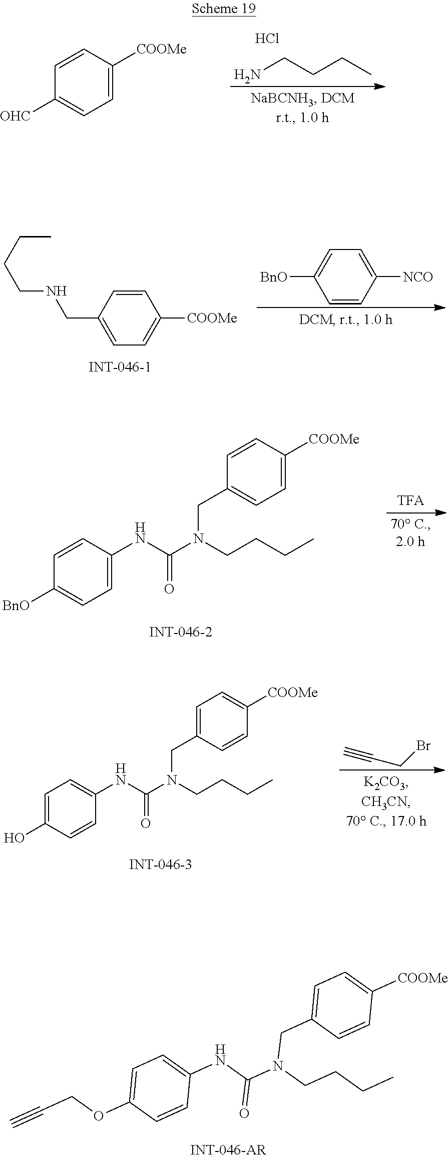

### Step 1: methyl 4-[(butylamino)methyl]benzoate

The procedure was the same as benzyl (2S)-2-[(2S)-6-{bicycle[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-024-OBn), but the reaction solvent was DCM, and after the reaction, the mixture was concentrated and purified by reverse phase directly. 1.00 g of Methyl 4-formylbenzoate was used, 400.00 mg of desired product was obtained as light yellow oil. LC/MS: mass calcd. For C₁₃H₁₉NO₂: 221.14, found: 222.15 [M+H]⁺.

### Step 2: Synthesis of methyl 4-[({[4-(benzyloxy)phenyl]carbamoyl}(butyl)amino) methyl]benzoate

Methyl 4-[(butylamino)methyl]benzoate (380.00 mg, 1.72 mmol, 1.00 equiv) and 1-(benzyloxy)-4-isocyanatobenzene (425.46 mg, 1.90 mmol, 1.10 equiv) were dissolved in DCM (3.00 mL). The resulting mixture was stirred for 1.0 h at room temperature. The resulting mixture was concentrated under reduced pressure and purified by silica gel column chromatography, eluted with PE and EA (PE:EA=2:1). The fractions were combined and concentrated to afford methyl 4-[({[4-(benzyloxy)phenyl]carbamoyl} (butyl)amino)methyl]benzoate (560.00 mg, 73.03% yield) as a light brown oil. LC/MS: mass calcd. For C₂₇H₃₀N₂O₄: 446.22, found: 447.30[M+H]⁺.

### Step 3: Synthesis of methyl 4-({butyl[(4-hydroxyphenyl)carbamoyl]amino} methyl)benzoate

Methyl 4-[({[4-(benzyloxy)phenyl]carbamoyl}(butyl)amino)methyl]benzoate (500.00 mg, 1.12 mmol, 1.00 equiv) was dissolved in trifluoroacetaldehyde (3.00 mL) and the resulting mixture was stirred for 2.0 h at 70° C. The resulting mixture was concentrated under reduced pressure to afford methyl 4-({butyl[(4-hydroxyphenyl)carbamoyl]amino}methyl)benzoate (500.00 mg, crude) as a brown oil. LC/MS: mass calcd. For C₂₀H₂₄N₂O₄: 356.17, found: 357.20[M+H]⁺.

### Step 4: Synthesis of methyl 4-{[butyl({[4-(prop-2-yn-1-yloxy)phenyl]carbamoyl}) amino]methyl}benzoate

To a stirred solution of methyl 4-({butyl[(4-hydroxyphenyl)carbamoyl]amino} methyl)benzoate (400.00 mg, 1.12 mmol, 1.00 equiv) in ACN (3.00 mL), propargyl bromide (200.26 mg, 1.68 mmol, 1.50 equiv) and K₂CO₃ (930.62 mg, 6.73 mmol, 6.00 equiv) were added and the resulting solution was stirred for 17.0 h at 70° C. The resulting mixture was filtered, the filter cake was washed with ACN (3×2 mL). The filtrate was concentrated under reduced pressure and purified by reverse flash chromatography with the following conditions: column, C18 silica gel; mobile phase, 0.5% NH₄HCO₃ in water and CH₃CN, 25% to 35% gradient in 20 min; detector, UV 254 nm and 220 nm. The fractions were combined and concentrated to afford methyl 4-{[butyl({[4-(prop-2-yn-1-yloxy)phenyl]carbamoyl})amino]methyl}benzoate (180.00 mg, 40.66% yield) as a light brown oil. LC/MS: mass calcd. For C₂₃H₂₆N₂O₄: 394.18, found: 395.15 [M+H]⁺.

### Example 20. Synthesis of (2S)-2-[(2S)-2-[(2S)-2-{[(2S,3aS,7aS)-1-(tert-butoxycarbonyl)-octahydroindol-2-yl]formamido}-6-[isopropyl(methyl)amino]hexanamido]-3-phenylpropanamido]-4-methylpentanoic acid (INT-047)

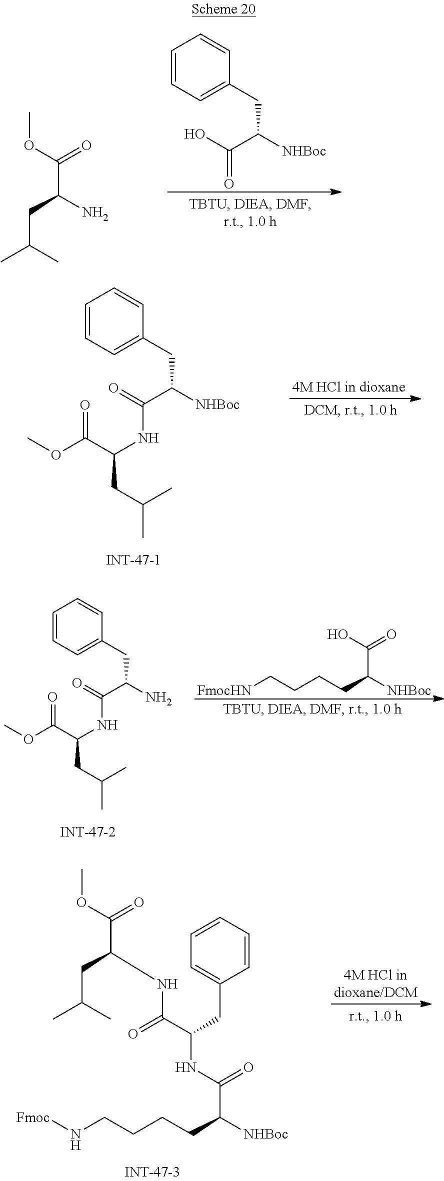

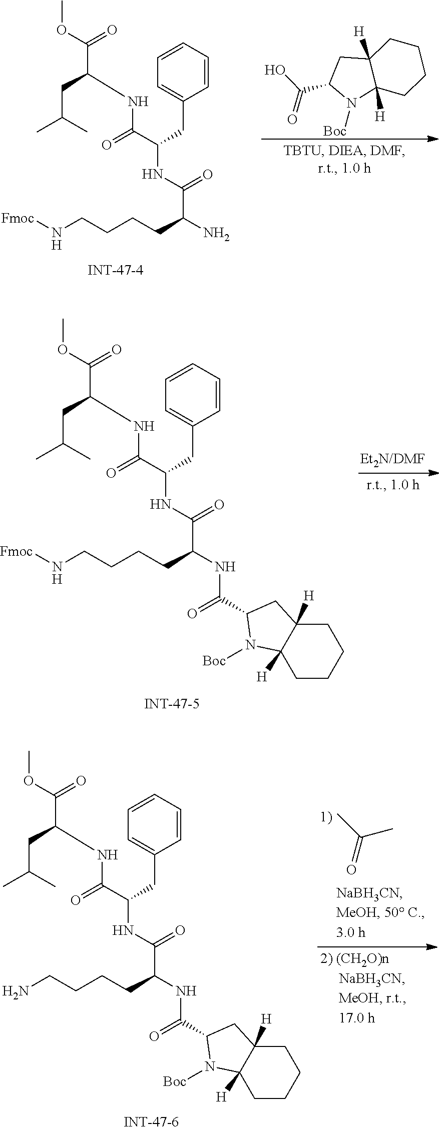

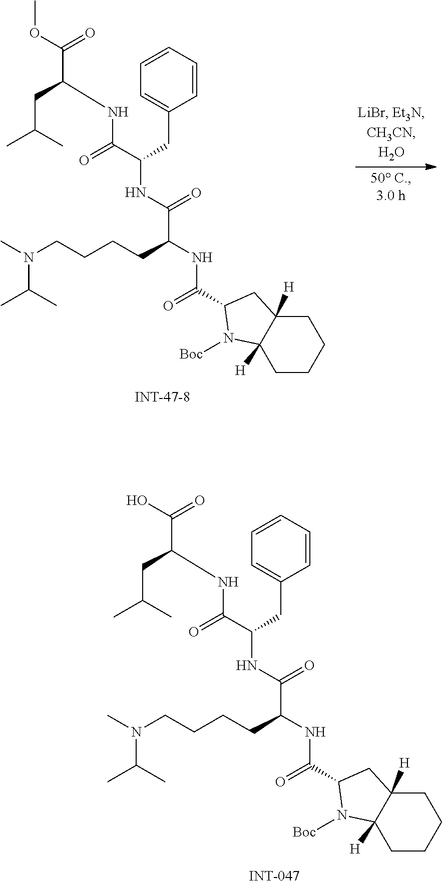 [SMILES: COC(=O)[C@H](CC(C)C)NC(=O)[C@H](Cc1ccccc1)NC(=O)[C@H](CCCCN(C)C(C)C)NC(=O)[C@@H]1C[C@@H]2CCCC[C@@H]2N1C(=O)OC(C)(C)C ; CC(C)C[C@H](NC(=O)[C@H](Cc1ccccc1)NC(=O)[C@H](CCCCN(C)C(C)C)NC(=O)[C@@H]1C[C@@H]2CCCC[C@@H]2N1C(=O)OC(C)(C)C)C(=O)O]

### Step 1: Synthesis of methyl (2S)-2-[(2S)-2-[(tert-butoxycarbonyl)amino]-3-phenylpropanamido]-4-methylpentanoate

The procedure was the same as methyl (2S)-2-[(4-tert-butylphenyl)formamido]-3-phenylpropanoate (INT-14.1). 5.00 of leucine methyl ester was used, 8.20 g of desired product was obtained as white solid (60.67% yield). LC/MS: mass calcd. For C₂₁H₃₂N₂O₅: 392.23, found: 393.25[M+H]⁺.

### Step 2: Synthesis of methyl (2S)-2-[(2S)-2-amino-3-phenylpropanamido]-4-methylpentanoate

The procedure was the same as methyl (2S)-2-[(2S)-2-amino-6-{[(9H-fluoren-9-yl)methoxy)carbonyl]amino}hexanamido]-3-hydroxypropanoate (INT-14.6), but after the reaction, the mixture was concentrated. The crude was used for next step directly. 8.10 g of methyl (2S)-2-[(2S)-2-[(tert-butoxycarbonyl)amino]-3-phenylpropanamido]-4-methylpentanoate was used, 8.10 g crude of desired product was obtained as brown oil. LC/MS: mass calcd. For C₁₆H₂₄N₂O₃: 292.18, found: 293.15 [M+H]⁺.

### Step 3: Synthesis of methyl (2S)-2-[(2S)-2-[(2S)-2-[(tert-butoxycarbonyl)amino]-6-{[(9H-fluoren-9-ylmethoxy)carbonyl]amino}hexanamido]-3-phenylpropanamido]-4-methylpentanoate

The procedure was the same as methyl (2S)-2-[(4-tert-butylphenyl)formamido]-3-phenylpropanoate (INT-14.1). 10.40 g of (2S)-2-[(tert-butoxycarbonyl) amino]-6-{[(9H-fluoren-9-ylmethoxy)carbonyl]amino}hexanoic acid was used, 14.00 g of desired product was obtained as white solid (78.11% yield). LC/MS: mass calcd. For C₄₂H₅₄N₄O₈:742.39, found: 765.65 [M+Na]⁺.

### Step 4: Synthesis of methyl (2S)-2-[(2S)-2-[(2S)-2-amino-6-{[(9H-fluoren-9-ylmethoxy)carbonyl]amino}hexanamido]-3-phenylpropanamido]-4-methylpentanoate

The procedure was the same as methyl (2S)-2-[(2S)-2-amino-6-{[(9H-fluoren-9-yl)methoxy)carbonyl]amino}hexanamido]-3-hydroxypropanoate (INT-14.6), but after the reaction, the mixture was concentrated. The crude was used for next step directly. 4.00 g of methyl (2S)-2-[(2S)-2-[(2S)-2-[(tert-butoxycarbonyl)amino]-6-{[(9H-fluoren-9-ylmethoxy)carbonyl]amino}hexanamido]-3-phenylpropanamido]-4-methylpentanoate was used, 4.00 g crude of desired product was obtained as brown oil. LC/MS: mass calcd. For C₃₇H₄₆N₄O₆: 642.34, found: 643.35 [M+H]⁺.

### Step 5: Synthesis of tert-butyl (2S,3aS,7aS)-2-{[(1S)-5-{[(9H-fluoren-9-ylmethoxy)carbonyl]amino}-1-{[(1S)-1-{[(2S)-1-methoxy-4-methyl-1-oxopentan-2-yl]carbamoyl}-2-phenylethyl]carbamoyl}pentyl]carbamoyl}-octahydroindole-1-carboxylate

The procedure was the same as methyl (2S)-2-[(4-tert-butylphenyl)formamido]-3-phenylpropanoate (INT-14.1). 3.46 g of methyl (2S)-2-[(2S)-2-[(2S)-2-amino-6-{[(9H-fluoren-9-ylmethoxy)carbonyl]amino}hexanamido]-3-phenylpropanamido]-4-methylpentanoate was used, 3.70 g of desired product was obtained as pink solid. LC/MS: mass calcd. For C₅₁H₆₇N₅O₉: 893.49, found: 894.55[M+H]⁺.

### Step 6: Synthesis of tert-butyl (2S,3aS,7aS)-2-{[(1S)-5-amino-1-{[(1S)-1-{[(2S)-1-methoxy-4-methyl-1-oxopentan-2-yl]carbamoyl}-2-phenylethyl]carbamoyl}pentyl]carbamoyl}-octahydroindole-1-carboxylate

The procedure was the same as methyl (2S)-2-[(2S)-6-amino-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-14.11). 3.65 g of tert-butyl (2S,3aS,7aS)-2-{[(1S)-5-{[(9H-fluoren-9-ylmethoxy)carbonyl] amino}-1-{[(1S)-1-{[(2S)-1-methoxy-4-methyl-1-oxopentan-2-yl]carbamoyl}-2-phenylethyl]carbamoyl}pentyl]carbamoyl}-octahydroindole-1-carboxylate was used, 1.30 g of desired product was obtained as white solid (42.66% yield). LC/MS: mass calcd. For C₃₆H₅₇N₅O₇: 671.43, found: 672.70 [M+H]⁺.

### Step 7: Synthesis of tert-butyl (2S,3aS,7aS)-2-{[(1S)-5-[isopropyl(methyl)amino]-1-{[(1S)-1-{[(2S)-1-methoxy-4-methyl-1-oxopentan-2-yl]carbamoyl}-2-phenylethyl]carbamoyl}pentyl]carbamoyl}-octahydroindole-1-carboxylate

The procedure was the same as methyl (2S)-2-[(2S)-6-{bicycle [2.2.1]heptan-2-yl(methyl)amino}-2-[2-(2-{2-[(4-tert-butylphenyl)formamido]acetamido}acetamido)acetamido]hexanamido]-3-hydroxypropanoate and (2S)-2-[(2S)-6-{bicycle[2.2.1]heptan-2-yl(methyl)amino}-2-[2-(2-{2-[(4-tert-butylphenyl)formamido]acetamido}acetamido)acetamido]hexanamido]-3-hydroxypropanoic acid (INT-33-2). 900.00 mg of tert-butyl (2S,3aS,7aS)-2-{[(1S)-5-amino-1-{[(1S)-1-{[(2S)-1-methoxy-4-methyl-1-oxopentan-2-yl]carbamoyl}-2-phenylethyl]carbamoyl}pentyl]carbamoyl}-octahydroindole-1-carboxylate was used, 310.00 mg of desired product was obtained as yellow solid. LC/MS: mass calcd. For C₄₀H₆₅N₅O₇: 727.49, found: 728.75 [M+H]⁺.

### Step 8: Synthesis of (2S)-2-[(2S)-2-[(2S)-2-{[(2S,3aS,7aS)-1-(tert-butoxycarbonyl)-octahydroindol-2-yl]formamido}-6-[isopropyl(methyl)amino]hexanamido]-3-phenylpropanamido]-4-methylpentanoic acid

The procedure was the same as (2S)-2-[(2S)-6-{bicyclo[2.2.1] heptan-2-yl(2-methylpropyl)amino}-2-[2-(2-{2-[(4-tert-butylphenyl)formamido]ethoxy}ethoxy)acetamido]hexanamido]-3-hydroxypropanoic acid (INT-34-PEG2-C(O)-PTBPh-NIB). But the reaction temperature was 50° C., and reaction time was 3.0 h. 170.00 mg of tert-butyl (2S,3aS,7aS)-2-{[(1S)-5-[isopropyl(methyl)amino]-1-{[(1S)-1-{[(2S)-1-methoxy-4-methyl-1-oxopentan-2-yl]carbamoyl}-2-phenylethyl]carbamoyl}pentyl]carbamoyl}-octahydroindole-1-carboxylate was used, 100.00 mg of desired product was obtained as white solid (60.98% yield). LC/MS: mass calcd. For C₃₉H₆₃N₅O₇: 713.47, found: 714.75 [M+H]⁺.

### Example 21. Synthesis of tert-butyl 4-{4-[(3S,4R)-4-(dimethylamino)-1-(7-fluoro-2,3-dihydro-1H-inden-1-yl)pyrrolidin-3-yl]phenyl}piperazine-1-carboxylate (INT-048A-Boc)

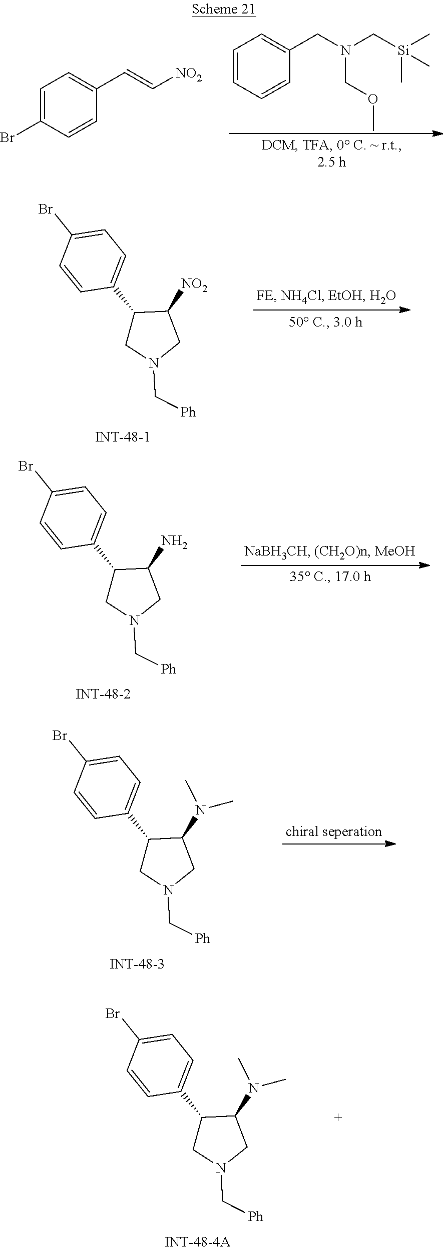 [SMILES: O=[N+]([O-])/C=C/c1ccc(Br)cc1 ; COCN(Cc1ccccc1)C[Si](C)(C)C ; O=[N+]([O-])[C@H]1CN(Cc2ccccc2)C[C@@H]1c1ccc(Br)cc1 ; N[C@H]1CN(Cc2ccccc2)C[C@@H]1c1ccc(Br)cc1 ; CN(C)[C@H]1CN(Cc2ccccc2)C[C@@H]1c1ccc(Br)cc1 ; CN(C)[C@H]1CN(Cc2ccccc2)C[C@@H]1c1ccc(Br)cc1]

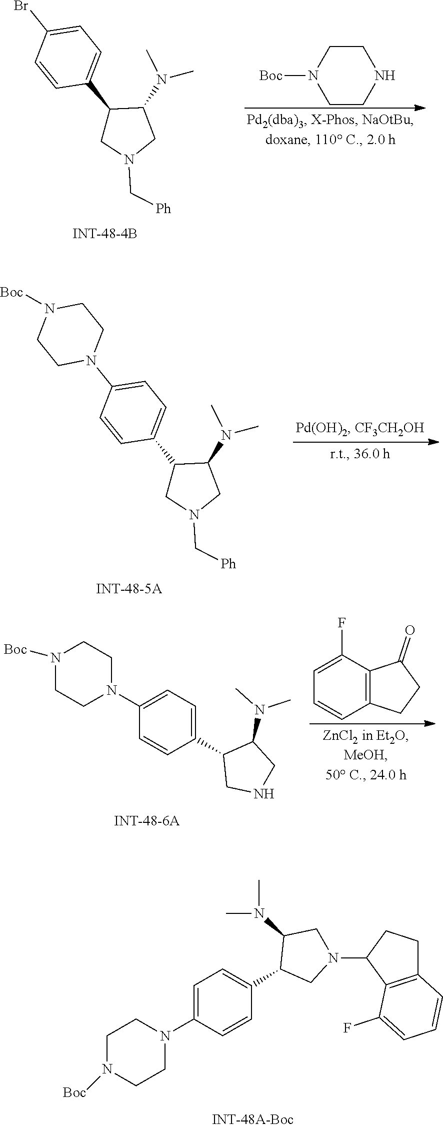

### Step 1: Synthesis of (3S,4R)-1-benzyl-3-(4-bromophenyl)-4-nitropyrrolidine

To a stirred solution of 1-bromo-4-[(E)-2-nitroethenyl]benzene (5.00 g, 21.93 mmol, 1.00 equiv) and TFA (5.00 mL) in DCM (50.00 mL) were added benzyl (methoxymethyl)[(trimethylsilyl)methyl] amine (6.25 g, 26.33 mmol, 1.20 equiv) in DCM (10.00 mL) dropwise at 0° C. The resulting mixture was stirred for 1.0 h at 0° C. The resulting mixture was allowed to warm to room temperature and stirred for 1.5 h at room temperature. The reaction was quenched by the addition of sat. NaHCO₃ (aq.) (200 mL) at 0° C. The resulting mixture was extracted with CH₂Cl₂ (3×150 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (5:1) to afford (3S, 4R)-1-benzyl-3-(4-bromophenyl)-4-nitropyrrolidine (7.20 g, 87.27% yield) as a white solid. LC/MS: mass calcd. For C₁₇H₁₇BrN₂O₂: 360.05, found: 361.10, 363.10[M+H, M+2+H]⁺.

### Step 2: Synthesis of (3R,4S)-1-benzyl-4-(4-bromophenyl)pyrrolidin-3-amine

A mixture of (3S,4R)-1-benzyl-3-(4-bromophenyl)-4-nitropyrrolidine (3.50 g, 9.69 mmol, 1.00 equiv) and Fe powder (9.00 g, 161.16 mmol, 16.63 equiv), NH₄Cl (9.00 g, 168.25 mmol, 17.37 equiv) in H₂O (20.00 mL) and EtOH (80.00 mL) was stirred for 3.0 h at 50° C. The mixture was allowed to cool down to room temperature. The resulting mixture was filtered and the filter cake was washed with EtOH (3×30 mL). The filtrate was concentrated under reduced pressure. The residue was diluted with water (100 mL). The resulting mixture was extracted with CH₂Cl₂ (3×150 mL). The combined organic layers were washed with brine (2×100 mL), dried over anhydrous Na₂SO₄. After filtration, the filtrate was concentrated under reduced pressure. This resulted in (3R,4S)-1-benzyl-4-(4-bromophenyl)pyrrolidin-3-amine (2.90 g, crude) as a yellow solid. LCMS: mass calcd. For C₁₇H₁₉BrN₂: 330.07, found: 331.10, 333.10[M+H, M+2+H]⁺.

### Step 3: Synthesis of (3R,4S)-1-benzyl-4-(4-bromophenyl)-N,N-dimethylpyrrolidin-3-amine

The procedure was the same as benzyl (2S)-2-[(2S)-6-{bicycle[2.2.1]heptan-2-yl(methyl)amino}-2-[(2S)-2-[(2S)-2-[(2S)-2-[(4-tert-butylphenyl)formamido]-3-phenylpropanamido]propanamido]-4-methylpentanamido]hexanamido]-3-hydroxypropanoate (INT-024-OBn), but the reaction temperature was 35° C., reaction time was 17.0 h. 2.50 g of (3R,4S)-1-benzyl-4-(4-bromophenyl)pyrrolidin-3-amine was used, 1.30 g of desired product was obtained as a white oil (47.94% yield). LC/MS: mass calcd. For C₁₉H₂₃BrN₂: 358.10, found: 359.20, 361.20[M+H, M+2+H]⁺.

### Step 4: Synthesis of (3R,4S)-1-benzyl-4-(4-bromophenyl)-N,N-dimethylpyrrolidin-3-amine and (3S,4R)-1-benzyl-4-(4-bromophenyl)-N,N-dimethylpyrrolidin-3-amine

The racemate mixture product (3R,4S)-1-benzyl-4-(4-bromophenyl)-N,N-dimethylpyrrolidin-3-amine (1.30 g, 3.62 mmol, 1.00 equiv) was separated by SFC with the following conditions: Column: CHIRAL ART Amylose-C NEO, 5*25 cm, 10 μm; Mobile Phase A: CO2, Mobile Phase B: MEOH (0.1% 2M NH3-MEOH); Flow rate: 200 mL/min; Gradient: isocratic 20% B; Column Temperature (° C.): 35; Back Pressure (bar): 100; Wave Length: 220 n; RT1 (min): 6.29; RT2 (min): 8.54; Sample Solvent: MeOH—Preparative; Injection Volume: 1 mL; Number Of Runs: 35. The fractions were combined and evaporated to afford the (3R,4S)-1-benzyl-4-(4-bromophenyl)-N,N-dimethylpyrrolidin-3-amine (450.00 mg, 34.62% yield) as a white oil and (3S,4R)-1-benzyl-4-(4-bromophenyl)-N,N-dimethylpyrrolidin-3-amine (430.00 mg, 33.08% yield) as a white oil. 4A: LC/MS: mass calcd. For C₁₉H₂₃BrN₂: 358.10, found: 359.15, 361.15[M+H, M+2+H]⁺. 4B: LC/MS: mass calcd. For C₁₉H₂₃BrN₂: 358.10, found: 359.15, 361.15[M+H+2]⁺.

### Step 5: Synthesis of tert-butyl 4-{4-[(3S,4R)-1-benzyl-4-(dimethylamino)pyrrolidin-3-yl] phenyl}piperazine-1-carboxylate

A mixture of (3R,4S)-1-benzyl-4-(4-bromophenyl)-N,N-dimethylpyrrolidin-3-amine (400.00 mg, 1.11 mmol, 1.00 equiv) and tert-butyl piperazine-1-carboxylate (414.00 mg, 2.22 mmol, 2.00 equiv), Pd₂(dba)₃ (103.00 mg, 0.11 mmol, 0.10 equiv), XPhos (106.00 mg, 0.22 mmol, 0.20 equiv), Sodium t-butoxide (290.00 mg, 3.02 mmol, 2.71 equiv) in dioxane (10.00 mL) was stirred for 2.0 h at 110° C. under nitrogen atmosphere. The mixture was allowed to cool down to room temperature. The resulting mixture was filtered and the filter cake was washed with 1,4-dioxane (2×5 mL). The filtrate was concentrated under reduced pressure. The residue was purified by silica gel column chromatography, eluted with CH₂Cl₂/MeOH (10:1) to afford tert-butyl 4-{4-[(3S,4R)-1-benzyl-4-(dimethylamino)pyrrolidin-3-yl] phenyl}piperazine-1-carboxylate (430.00 mg, 83.13% yield) as a brown oil. LC/MS: mass calcd. For C₂₈H₄₀N₄O₂: 464.32, found: 465.30[M+H]⁺.

### Step 6: Synthesis of tert-butyl 4-{4-[(3S,4R)-4-(dimethylamino)pyrrolidin-3-yl] phenyl}piperazine-1-carboxylate

To a stirred solution of tert-butyl 4-{4-[(3S,4R)-1-benzyl-4-(dimethylamino)pyrrolidin-3-yl]phenyl}piperazine-1-carboxylate (420.00 mg, 0.90 mmol, 1.00 equiv) in CF₃CH₂OH (6.00 mL) were added Pd(OH)₂/C (84.00 mg, 20% w/w) in portions at room temperature. The resulting mixture was stirred for 36.0 h at room temperature under hydrogen atmosphere. The resulting mixture was filtered and the filter cake was washed with CF₃CH₂OH (2×5 mL). The filtrate was concentrated under reduced pressure. This resulted in tert-butyl 4-{4-[(3S,4R)-4-(dimethylamino)pyrrolidin-3-yl]phenyl}piperazine-1-carboxylate (370.00 mg, crude) as a brown oil. LC/MS: mass calcd. For C₂₁H₃₄N₄O₂: 374.27, found: 375.30[M+H]⁺.

### Step 7: Synthesis of tert-butyl 4-{4-[(3S,4R)-4-(dimethylamino)-1-(7-fluoro-2,3-dihydro-1H-inden-1-yl)pyrrolidin-3-yl]phenyl}piperazine-1-carboxylate

To a stirred mixture of tert-butyl 4-{4-[(3S,4R)-4-(dimethylamino)pyrrolidin-3-yl]phenyl}piperazine-1-carboxylate (350.00 mg, 0.94 mmol, 1.00 equiv) and 7-fluoro-2,3-dihydroinden-1-one (210.00 mg, 1.40 mmol, 1.50 equiv) in MeOH (4.00 mL) were added NaBH₃CN (180.00 mg, 7.83 mmol, 8.38 equiv), zinc chloride (1M in diethyl ether, 4 mL) in portions at room temperature. The resulting mixture was stirred for 24.0 h at 50° C. The mixture was allowed to cool down to room temperature. The resulting mixture was filtered and the filter cake was washed with MeOH (3×5 mL). The filtrate was concentrated under reduced pressure. The residue was purified by silica gel column chromatography, eluted with CH₂Cl₂/MeOH (20:1) to afford tert-butyl 4-{4-[(3S,4R)-4-(dimethylamino)-1-(7-fluoro-2,3-dihydro-1H-inden-1-yl)pyrrolidin-3-yl]phenyl}piperazine-1-carboxylate (390.00 mg, 79.10% yield) as a brown oil. LC/MS: mass calcd. For C₃₀H₄₁FN₄O₂: 508.32, found: 509.30[M+H]⁺.

### Example 22. Synthesis of N-[(4,6-dimethyl-2-oxo-1H-pyridin-3-yl)methyl]-5-[ethyl(oxan-4-yl)amino]-4-methyl-4′-(piperazin-1-ylmethyl)-[1,1′-biphenyl]-3-carboxamide (INT-052)

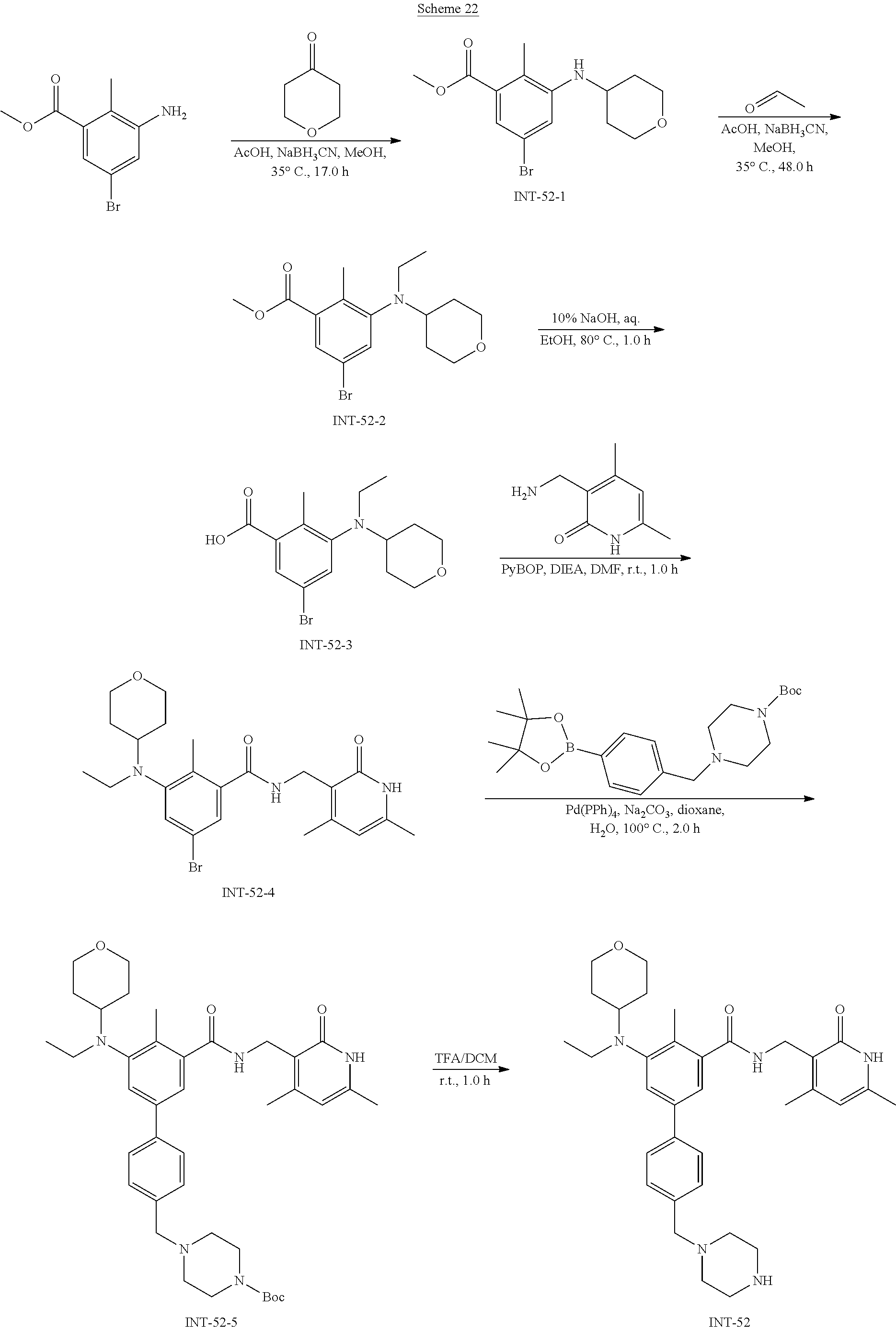

### Step 1: Synthesis of methyl 5-bromo-2-methyl-3-(oxan-4-ylamino)benzoate

To a solution of methyl 3-amino-5-bromo-2-methylbenzoate (5.00 g, 20.48 mmol, 1.00 equiv) in MeOH (50.00 mL) was added tetrahydro-4H-pyran-4-one (4.10 g, 40.97 mmol, 2.00 equiv), AcOH (2.46 g, 40.97 mmol, 2.00 equiv), NaBH₃CN (3.86 g, 61.452 mmol, 3.00 equiv). The resulting mixture was stirred at 35° C. for 17.0 h. The resulting mixture was concentrated under vacuum. The residue obtained was purified by silica gel chromatography (0-20% ethyl acetate/petroleum ether) to afford methyl 5-bromo-2-methyl-3-(oxan-4-ylamino)benzoate (4.45 g, 66.19% yield) as white solid. LC/MS: mass calcd. for Cl₄H₁₈BrNO₃: 327.04, found: 328.05, 330.05 [M+H, M+2+H]⁺.

### Step 2: Synthesis of methyl 5-bromo-3-[ethyl(oxan-4-yl)amino]-2-methylbenzoate

The procedure was the same as methyl 5-bromo-2-methyl-3-(oxan-4-ylamino)benzoate, but the reaction time was 48.0 h. 4.45 g of methyl 5-bromo-2-methyl-3-(oxan-4-ylamino)benzoate was used, 3.40 g of desired product was obtained as yellow solid (70.39% yield). LC/MS: mass calcd. for C₁₆H₂₂BrNO₃: 355.07, found: 356.25, 358.25 [M+H, M+2+H]⁺.

### Step 3: Synthesis of 5-Bromo-3-[ethyl(oxan-4-yl)amino]-2-methylbenzoic acid

To a solution of methyl 5-bromo-3-[ethyl(oxan-4-yl)amino]-2-methylbenzoate (3.40 g, 9.54 mmol, 1.00 equiv) in EtOH (40.00 mL) was added 10% NaOH aq. (19.09 mL, 38.18 mmol, 4.00 equiv).

The resulting mixture was stirred at 80° C. for 1.0 h. The resulting mixture was concentrated under reduced pressure. The residue was dissolved in H₂O (40 mL). The mixture was acidified to pH 3˜5 with 2 M HCl, the reaction mixture was extracted by EA (3×40 mL), the organic phases were combined and washed by H₂O (1×20 mL) and NaCl (1×20 mL), dried over anhydrous Na₂SO₄. After filtration, the filtrate was concentrated under reduced pressure. 5-Bromo-3-[ethyl(oxan-4-yl)amino]-2-methylbenzoic acid (3.20 g, 97.98% yield) was obtained as yellow solid. LC/MS: mass calcd. for C₁₅H₂₀BrNO₃: 341.06, found: 342.25, 344.25 [M+H, M+2+H]⁺.

### Step 4: Synthesis of 5-Bromo-N-[(4,6-dimethyl-2-oxo-1H-pyridin-3-yl)methyl]-3-[ethyl(oxan-4-yl)amino]-2-methylbenzamide

The procedure was the same as methyl (2S)-2-[(2S)-2-[2-(2-{2-[(4-tert-butylphenyl)formamido]acetamido}acetamido)acetamido]-6-{[(9H-fluoren-9-ylmethoxy)carbonyl]amino}hexanamido]-3-hydroxypropanoate (INT-32-102). 3.00 g of 5-bromo-3-[ethyl(oxan-4-yl)amino]-2-methylbenzoic acid was used, 4.00 g of desired product was obtained as white solid (95.78% yield). LC/MS: mass calcd. for C₂₃H₃₀BrN₃O₃: 475.14, found: 476.30, 478.30 [M+H, M+2+H]⁺.

### Step 5: Synthesis of tert-butyl 4-[(3′-{[(4,6-dimethyl-2-oxo-1H-pyridin-3-yl) methyl]carbamoyl}-5′-[ethyl(oxan-4-yl)amino]-4′-methyl-[1,1′-biphenyl]-4-yl)methyl]piperazine-1-carboxylate

To a solution of 5-bromo-N-[(4,6-dimethyl-2-oxo-1H-pyridin-3-yl)methyl]-3-[ethyl(oxan-4-yl)amino]-2-methylbenzamide (3.80 g, 7.98 mmol, 1.00 equiv) in 1,4-dioxane (32.00 mL) and H₂O (8.00 mL) was added tert-butyl 4-{[4-(4,4,5,5-tetramethyl-1,3,2-dioxaborolan-2-yl)phenyl]methyl}piperazine-1-carboxylate (3.21 g, 7.98 mmol, 1.00 equiv), Na₂CO₃ (2.54 g, 23.93 mmol, 3.00 equiv), Pd(PPh₃)₄ (0.92 g, 0.80 mmol, 0.10 equiv). Then the reaction was stirred for 2.0 h at 100° C. under N₂ atmosphere. The reaction was quenched with water (40 mL). The resulting mixture was extracted with ethyl acetate (3×40 mL). The organic layers were combined, dried over anhydrous sodium sulfate, filtered and concentrated.

The residue obtained was purified by silica gel chromatography (0-20% DCM/MeOH) to afford tert-butyl 4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazine-1-carboxylate (5.20 g, 97.03% yield) as yellow oil. LC/MS: mass calcd. for C₃₉H₅₃N₅O₅: 671.40, found: 672.45 [M+H]⁺.

### Step 6: Synthesis of N-[(4,6-dimethyl-2-oxo-1H-pyridin-3-yl)methyl]-5-[ethyl (oxan-4-yl)amino]-4-methyl-4′-(piperazin-1-ylmethyl)-[1,1′-biphenyl]-3-carboxamide

The procedure was the same as methyl (2S)-2-[(2S)-2-[(2S)-2-amino-5-(morpholin-4-yl)pentanamido]-6-{[(9H-fluoren-9-ylmethoxy)carbonyl]amino} hexanamido]-3-hydroxypropanoate (INT-50-6). 300.00 mg of tert-butyl 4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazine-1-carboxylate was used, 250.00 mg crude of desired product was obtained as yellow oil. LC/MS: mass calcd. for C₃₄H₄₅N₅O₃: 571.35, found: 572.65 [M+H]⁺.

### Example 23. Synthesis of 1-[7-hydroxy-1-(2-methanesulfonylphenyl)indolizin-3-yl]ethanone (INT-053)

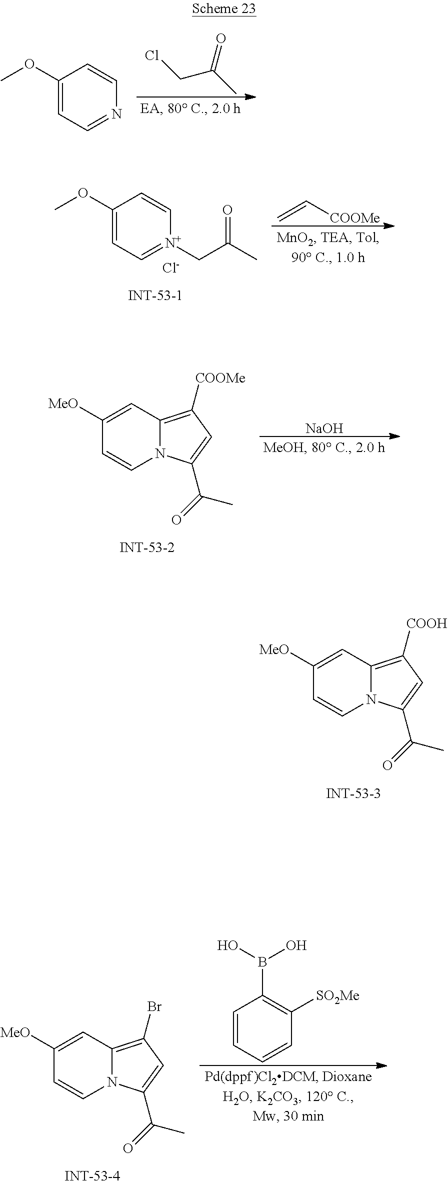

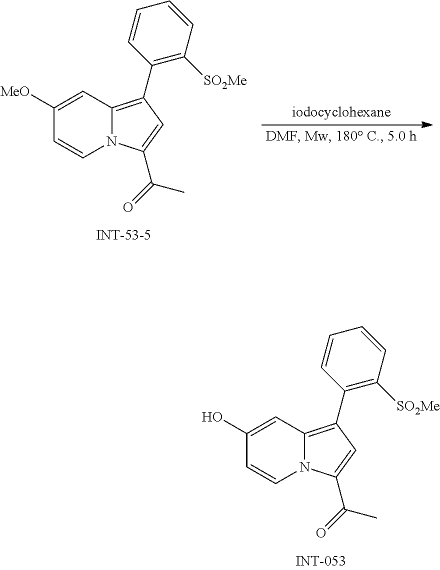 [SMILES: COc1ccn2c(C(C)=O)cc(-c3ccccc3S(C)(=O)=O)c2c1 ; CC(=O)c1cc(-c2ccccc2S(C)(=O)=O)c2cc(O)ccn12]

### Step 1: Synthesis of 4-methoxy-1-(2-oxopropyl)pyridin-1-ium chloride

4-methoxypyridine (1.20 g, 11.00 mmol, 1.00 equiv) and tonite (5.09 g, 54.98 mmol, 5.00 equiv) were dissolved in EA (20.00 mL). The resulting mixture was stirred for 2.0 h at 80° C. The resulting mixture was concentrated under vacuum to afford 4-methoxy-1-(2-oxopropyl)pyridin-1-ium chloride (2.20 g, crude) as a brown oil. LC/MS: mass calcd. For C₉H₁₂ClNO₂: 201.05, found: 166.10 [M−Cl]⁺.

### Step 2: Synthesis of methyl 3-acetyl-7-methoxyindolizine-1-carboxylate

To a stirred solution of 4-methoxy-1-(2-oxopropyl)pyridin-1-ium chloride (2.80 g, 13.89 mmol, 1.00 equiv) in toluene (30.00 mL), methyl acrylate (11.95 g, 138.85 mmol, 10.00 equiv), Et₃N (2.11 g, 20.83 mmol, 1.50 equiv) and MnO₂ (9.66 g, 111.08 mmol, 8.00 equiv) were added and the resulting mixture was stirred for 1.0 h at 90° C. The resulting mixture was filtered, the filter cake was washed with EA (3×8 mL). The filtrate was concentrated under reduced pressure and purified by silica gel column chromatography, eluted with PE and EA (PE:EA=4:1). The fraction were combined and concentrated to afford methyl 3-acetyl-7-methoxyindolizine-1-carboxylate (2.00 g, 52.43% yield) as a yellow solid. LC/MS: mass calcd. For C₁₃H₁₃NO₄: 247.08, found: 248.10[M+H]⁺.

### Step 3: Synthesis of 3-acetyl-7-methoxyindolizine-1-carboxylic acid

To a stirred solution of methyl 3-acetyl-7-methoxyindolizine-1-carboxylate (1.90 g, 7.69 mmol, 1.00 equiv) in MeOH (20.00 mL) and THF (20.00 mL) was added NaOH (2M, 38.42 mL, 10.00 equiv).

The resulting mixture was stirred for 2.0 h at 80° C. The resulting mixture was concentrated under reduced pressure. The residue was dissolved in H₂O (15 mL). The mixture was acidified to pH 3˜4 with 2 M HCl. The precipitated solids were collected by filtration and washed with H₂O (3×10 mL), dried under vacuum to afford 3-acetyl-7-methoxyindolizine-1-carboxylic acid (1.00 g, 52.45% yield) as a yellow solid. LC/MS: mass calcd. For C₁₂H₁₁NO₄: 233.06, found: 234.25[M+H]⁺.

### Step 4: Synthesis of 1-(1-bromo-7-methoxyindolizin-3-yl)ethanone

To a stirred solution of 3-acetyl-7-methoxyindolizine-1-carboxylic acid (1.00 g, 4.29 mmol, 1.00 equiv) in dimethylformamide (8.00 mL), NaHCO₃ (1.08 g, 12.86 mmol, 3.00 equiv) and NBS (0.84 g, 4.72 mmol, 1.10 equiv) were added at 0° C. and the resulting mixture was stirred for 2.0 h at room temperature. The resulting mixture was poured in water (200 mL) and extracted with EA (3×100 mL). The combined organic layers were washed with H₂O (3×150 mL), dried over anhydrous Na₂SO₄. After filtration, the filtrate was concentrated under reduced pressure to afford 1-(1-bromo-7-methoxyindolizin-3-yl)ethanone (950.00 mg, 78.51% yield) as a dark green solid. LC/MS: mass calcd. For C₁₁H₁₀BrNO₂: 266.98, found: 268.00, 27.00 [M+H, M+2+H]⁺.

### Step 5: Synthesis of 1-[1-(2-methanesulfonylphenyl)-7-methoxyindolizin-3-yl]ethanone

Nitrogen gas was bubbled through a mixture of 1-(1-bromo-7-methoxyindolizin-3-yl)ethanone (900.00 mg, 3.36 mmol, 1.00 equiv), 2-methanesulfonylphenylboronic acid (1342.87 mg, 6.71 mmol, 2.00 equiv), 1,1′-bis(diphenylphosphino)ferrocene-palladium(II)dichloride dichloromethane complex (191.42 mg, 0.24 mmol, 0.07 equiv) and K₂CO₃ (1391.80 mg, 10.07 mmol, 3.00 equiv) in dioxane (8.00 mL) and H₂O (1.00 mL) (volume ratio of dioxane and H₂O=8:1). The resulting mixture was heated in a sealed tube under microwave condition (120° C., 30 min). The resulting mixture was extracted with EA (2×10 mL). The combined organic layers were washed with H₂O (3×15 mL), dried over anhydrous Na₂SO₄. After filtration, the filtrate was concentrated under reduced pressure. The crude was purified by silica gel column chromatography, eluted with PE and EA (PE:EA=2:1). The fraction were combined and concentrated to afford 1-[1-(2-methanesulfonylphenyl)-7-methoxyindolizin-3-yl]ethanone (540.00 mg, 42.16% yield) as a brown solid. LC/MS: mass calcd. For C₁₈H₁₇NO₄S: 343.08, found: 343.95 [M+H]⁺.

### Step 6: Synthesis of 1-[7-hydroxy-1-(2-methanesulfonylphenyl)indolizin-3-yl] ethanone

To a stirred solution of 1-[1-(2-methanesulfonylphenyl)-7-methoxyindolizin-3-yl] ethanone (200.00 mg, 0.58 mmol, 1.00 equiv) in DMF (3.00 mL), iodocyclohexane (1223.39 mg, 5.82 mmol, 10.00 equiv) was added and the resulting mixture was irradiated with microwave radiation for 5.0 h at 180° C.

The resulting mixture was poured into water (100 mL), extracted with EA (2×100 mL). The combined organic layers were washed with water (3×150 mL), dried over anhydrous Na₂SO₄. After filtration, the filtrate was concentrated under reduced pressure to afford 1-[7-hydroxy-1-(2-methanesulfonylphenyl)indolizin-3-yl]ethanone (200.00 mg, crude) as a dark green oil. LC/MS: mass calcd. For C₁₇H₁₅NO₄S: 329.07, found: 330.10 [M+H]⁺.

### Example 24. Synthesis of 4-[2-formyl-5-(1-methylpyrazol-4-yl)-3-[2-(1-methylpyrazol-4-yl)ethyl]imidazol-4-yl]benzonitrile (INT-054)

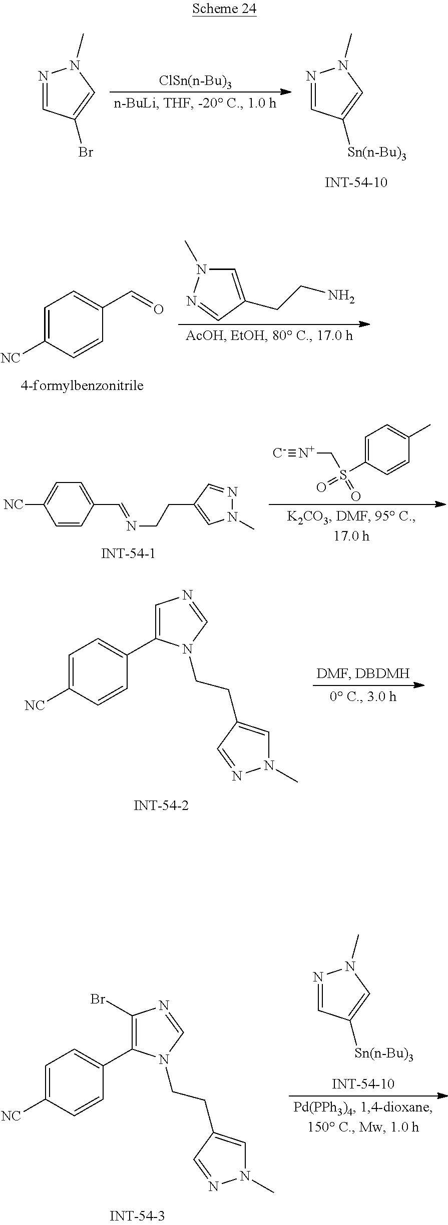

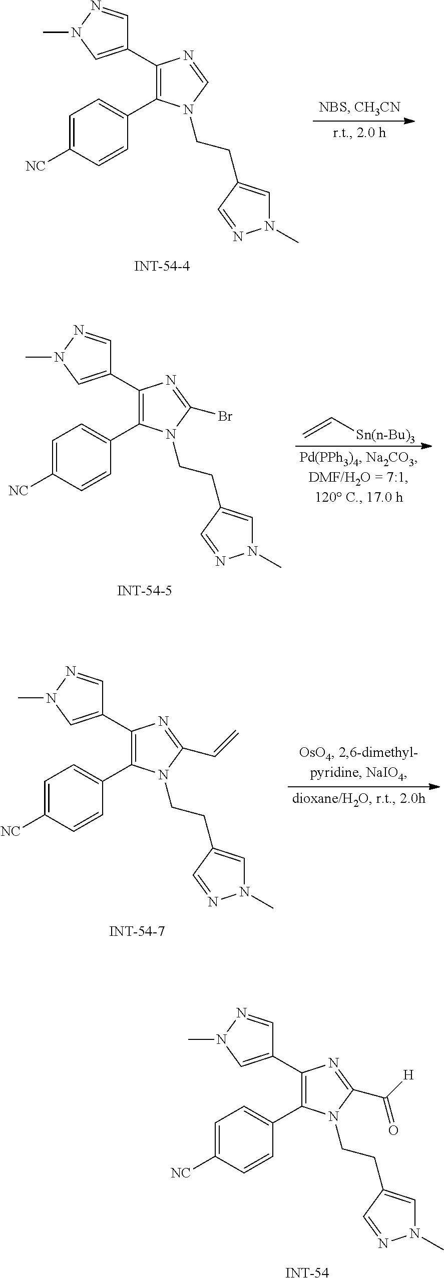

### Step 1: Synthesis of 1-methyl-4-(tributylstannyl)pyrazole

To a solution of 4-bromo-1-methylpyrazole (5.00 g, 31.06 mmol, 1.00 equiv) in THF (200.00 mL) was added dropwise n-BuLi in hexanes (2.5 M, 12.50 mL, 132.70 mmol, 4.27 equiv) at −20° C. and the mixture stirred at −20° C. to −10° C. for 0.5 h, followed by addition of Tin-Sn (8087.10 mg, 24.85 mmol, 0.80 equiv) dropwise. After 0.5 h, the reaction mixture was quenched with saturated NH₄Cl (aq), and the residue was extracted with EA (3×300 mL), dried over Na₂SO₄, filtrated and concentrated. The residue was purified by silica gel column chromatography, eluted with DCM/MeOH=10:1 to afford 1-methyl-4-(tributylstannyl)pyrazole (1.20 g, 10.41% yield) as yellow oil. LC/MS: mass calcd. For C₁₆H₃₂N₂Sn: 372.16, found: 373.20[M+H]⁺.

### Step 2: Synthesis of 4-[(1E)-{[2-(1-methylpyrazol-4-yl)ethyl]imino}methyl] benzonitrile

To a stirred solution of 4-formylbenzonitrile (3.20 g, 24.40 mmol, 1.00 equiv) in EtOH (50.00 mL) was added 2-(1-methylpyrazol-4-yl)ethanamine (3.05 g, 24.40 mmol, 1.00 equiv) and AcOH (4.19 mL, 73.12 mmol, 3.00 equiv) at room temperature. The resulting mixture was stirred for 17.0 h at 80° C.

The resulting mixture was concentrated under vacuum. 4-[(1E)-{[2-(1-methylpyrazol-4-yl)ethyl]imino}methyl] benzonitrile (5.80 g, crude) was obtained as yellow oil. LC/MS: mass calcd. For C₁₄H₁₄N₄: 238.12, found: 239.15[M+H]⁺.

### Step 3: Synthesis of 4-{3-[2-(1-methylpyrazol-4-yl)ethyl]imidazol-4-yl} benzonitrile

To a stirred solution of 4-[(1E)-{[2-(1-methylpyrazol-4-yl)ethyl]imino}methyl] benzonitrile (5.80 g, 24.34 mmol, 1.00 equiv) in DMF (200.00 mL) was added K₂CO₃ (6.73 g, 48.68 mmol, 2.00 equiv) and {[(4-methylbenzenesulfonyl)methyl] imino}methanide (7.13 g, 36.51 mmol, 1.50 equiv) at room temperature. The resulting mixture was stirred for 17.0 h at 95° C. After cooling down to r.t, the reaction mixture was poured into water (600 mL). The reaction mixture was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue obtained was purified by silica gel chromatography (0-10% methanol/dichloromethane) to afford the 4-{3-[2-(1-methylpyrazol-4-yl)ethyl]imidazol-4-yl}benzonitrile (6.10 g, 90.37% yield) was obtained as yellow solid. LC/MS: mass calcd. For C₁₆H₁₅N₅: 277.13, found: 278.30 [M+H]⁺.

### Step 4: Synthesis of 4-{5-bromo-3-[2-(1-methylpyrazol-4-yl)ethyl]imidazol-4-yl} benzonitrile

To a stirred solution of 4-{3-[2-(1-methylpyrazol-4-yl)ethyl]imidazol-4-yl} benzonitrile (6.10 g, 22.00 mmol, 1.00 equiv) in DMF (100.00 mL) was added 1,3-dibromo-5,5-dimethylimidazolidine-2,4-dione (3.14 g, 11.00 mmol, 0.50 equiv) in portions at 0° C. The resulting mixture was stirred for 3.0 h at 0° C. The reaction mixture was poured into water (300 mL). The reaction mixture was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue obtained was purified by silica gel chromatography (0-10% methanol/dichloromethane) to afford the 4-{5-bromo-3-[2-(1-methylpyrazol-4-yl)ethyl]imidazol-4-yl}benzonitrile (4.30 g, 54.88% yield) as yellow solid. LC/MS: mass calcd. For C₁₆H₁₄BrN₅: 355.04, found: 356.20, 358.20 [M+H, M+2+H]⁺.

### Step 5: Synthesis of 4-[5-(1-methylpyrazol-4-yl)-3-[2-(1-methylpyrazol-4-yl)ethyl] imidazol-4-yl]benzonitrile

To a stirred solution of 4-{5-bromo-3-[2-(1-methylpyrazol-4-yl)ethyl] imidazol-4-yl}benzonitrile (500.00 mg, 1.40 mmol, 1.00 equiv) in dioxane (8.00 mL) was added 1-methyl-4-(tributylstannyl)pyrazole (781.43 mg, 2.11 mmol, 1.50 equiv), Pd(PPh₃)₄ (162.19 mg, 0.14 mmol, 0.10 equiv) in portions at room temperature. The final reaction mixture was irradiated with microwave radiation for 1.5 h at 150° C. under N₂ atmosphere. The resulting mixture was concentrated under vacuum.

The residue was purified by silica gel column chromatography, eluted with DCM/MeOH (10:1) to afford 4-[5-(1-methylpyrazol-4-yl)-3-[2-(1-methylpyrazol-4-yl)ethyl]imidazol-4-yl]benzonitrile (270.00 mg, 53.82% yield) as yellow solid. LC/MS: mass calcd. For C₂₀H₁₉N₇:357.17, found: 358.20[M+H]⁺.

### Step 6: Synthesis of 4-[2-bromo-5-(1-methylpyrazol-4-yl)-3-[2-(1-methylpyrazol-4-yl)ethyl]imidazol-4-yl]benzonitrile

To a stirred solution of 4-[5-(1-methylpyrazol-4-yl)-3-[2-(1-methylpyrazol-4-yl) ethyl]imidazol-4-yl]benzonitrile (270.00 mg, 0.76 mmol, 1.00 equiv) in CH₃CN (10.00 mL) was added NBS (201.68 mg, 1.13 mmol, 1.50 equiv) in portions at room temperature. The resulting mixture was stirred for 2.0 h at room temperature. After cooling down to r.t., the reaction was removed under reduced pressure. The residue obtained was purified by silica gel chromatography (0-10% methanol/dichloromethane) to afford the 4-[2-bromo-5-(1-methylpyrazol-4-yl)-3-[2-(1-methylpyrazol-4-yl)ethyl]imidazol-4-yl]benzonitrile (210.00 mg, 63.71% yield) as yellow solid. LC/MS: mass calcd. For C₂₀H₁₈BrN₇: 435.08, found: 436.20, 438.20[M+H, M+2+H]⁺.

### Step 7: Synthesis of 4-[2-ethenyl-5-(1-methylpyrazol-4-yl)-3-[2-(1-methylpyrazol-4-yl)ethyl]imidazol-4-yl]benzonitrile

To a stirred solution of 4-[2-bromo-5-(1-methylpyrazol-4-yl)-3-[2-(1-methylpyrazol-4-yl)ethyl]imidazol-4-yl]benzonitrile (210.00 mg, 0.48 mmol, 1.00 equiv) in DMF/H₂O (7 mL:1 mL) was added tributyl(ethenyl)stannane (228.93 mg, 0.72 mmol, 1.50 equiv), Na₂CO₃ (153.04 mg, 1.44 mmol, 3.00 equiv) and Pd(PPh₃)₄ (55.62 mg, 0.045 mmol, 0.10 equiv) in portions at room temperature. The final reaction mixture was stirred for 17.0 h at 120° C. under N₂ atmosphere. The resulting mixture was concentrated under vacuum. The residue was purified by silica gel column chromatography, eluted with DCM/MeOH (10:1) to afford 4-[2-ethenyl-5-(1-methylpyrazol-4-yl)-3-[2-(1-methylpyrazol-4-yl)ethyl]imidazol-4-yl]benzonitrile (180.00 mg, 97.53% yield) as yellow solid. LC/MS: mass calcd. For C₂₂H₂₁N₇: 383.19, found: 384.35[M+H]⁺.

### Step 8: Synthesis of 4-[2-formyl-5-(1-methylpyrazol-4-yl)-3-[2-(1-methylpyrazol-4-yl)ethyl]imidazol-4-yl]benzonitrile

To a stirred solution of 4-[2-ethenyl-5-(1-methylpyrazol-4-yl)-3-[2-(1-methylpyrazol-4-yl)ethyl]imidazol-4-yl]benzonitrile (330.00 mg, 0.86 mmol, 1.00 equiv) in dioxane/H₂O (2 mL:2 mL) was added OSO₄ (21.88 mg, 0.09 mmol, 0.10 equiv), 2,6-Dimethylpyridine (184.43 mg, 1.72 mmol, 2.00 equiv) and NaIO₄ (368.14 mg, 1.72 mmol, 2.00 equiv) in portions at room temperature. The final reaction mixture was stirred for 2.0 h at room temperature. The reaction mixture was quenched with saturated Na₂SO₃ (aq), and the residue was extracted with EA (3×50 mL), dried over Na₂SO₄, filtrated and concentrated. The residue was purified by silica gel column chromatography, eluted with DCM/MeOH=10:1 to afford 4-[2-formyl-5-(1-methylpyrazol-4-yl)-3-[2-(1-methylpyrazol-4-yl)ethyl]imidazol-4-yl]benzonitrile (310.00 mg, 93.46% yield) as yellow oil. LC/MS: mass calcd. For C₂₁H₁₉N₇O: 385.17, found: 386.35[M+H]⁺.

Synthesis of Representative Polyamides

### Example 25. Synthesis of PA-003

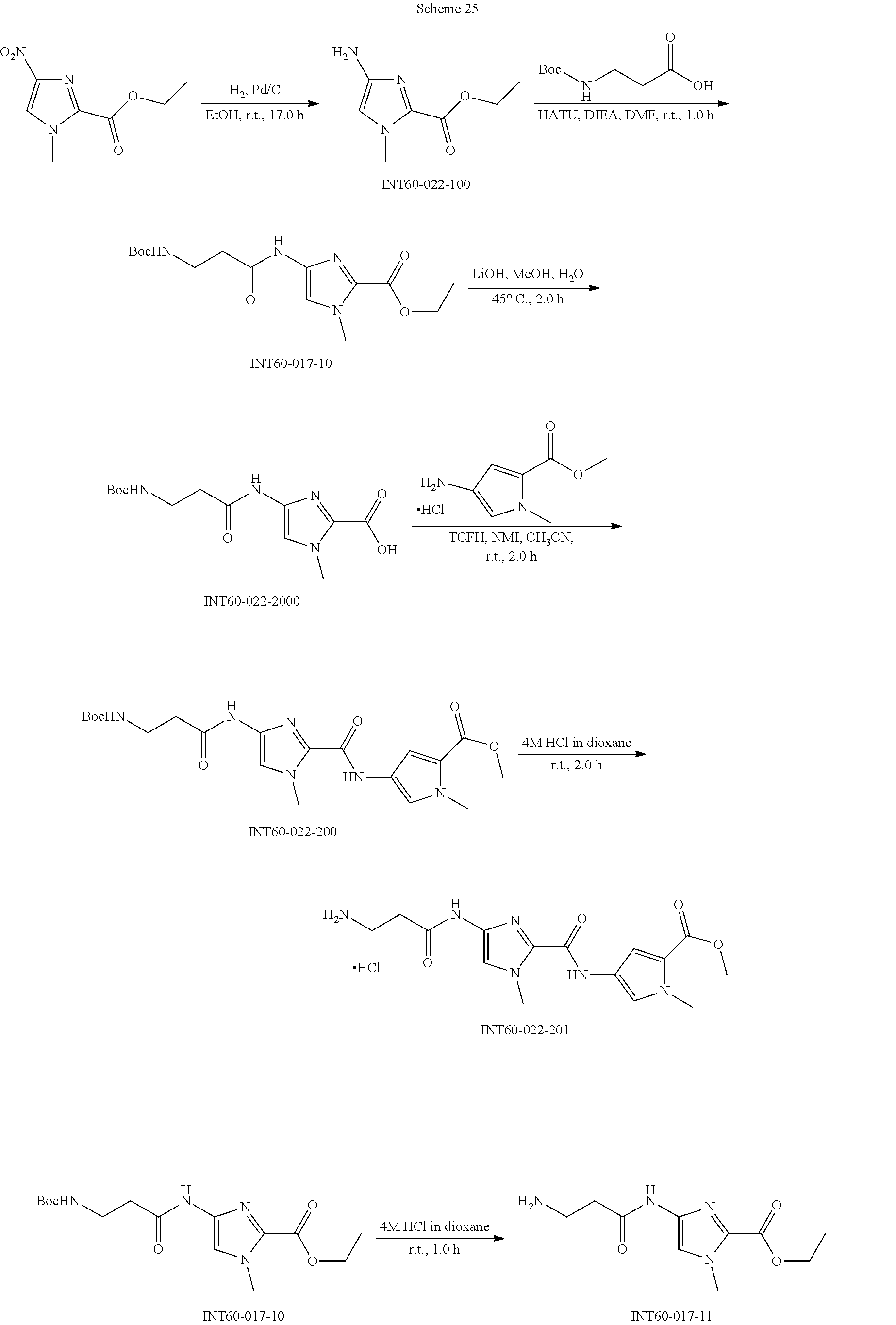

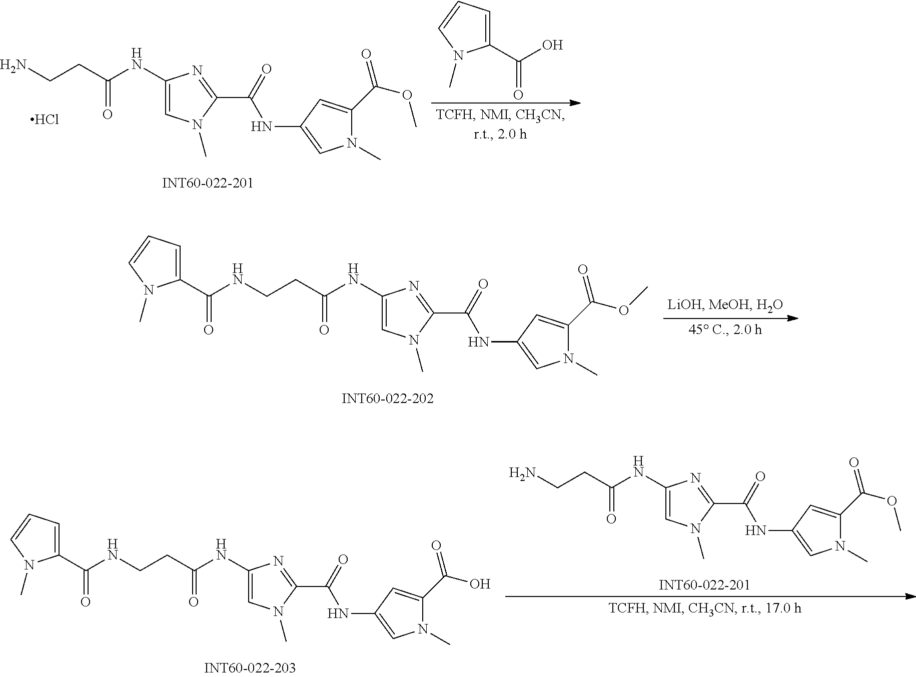

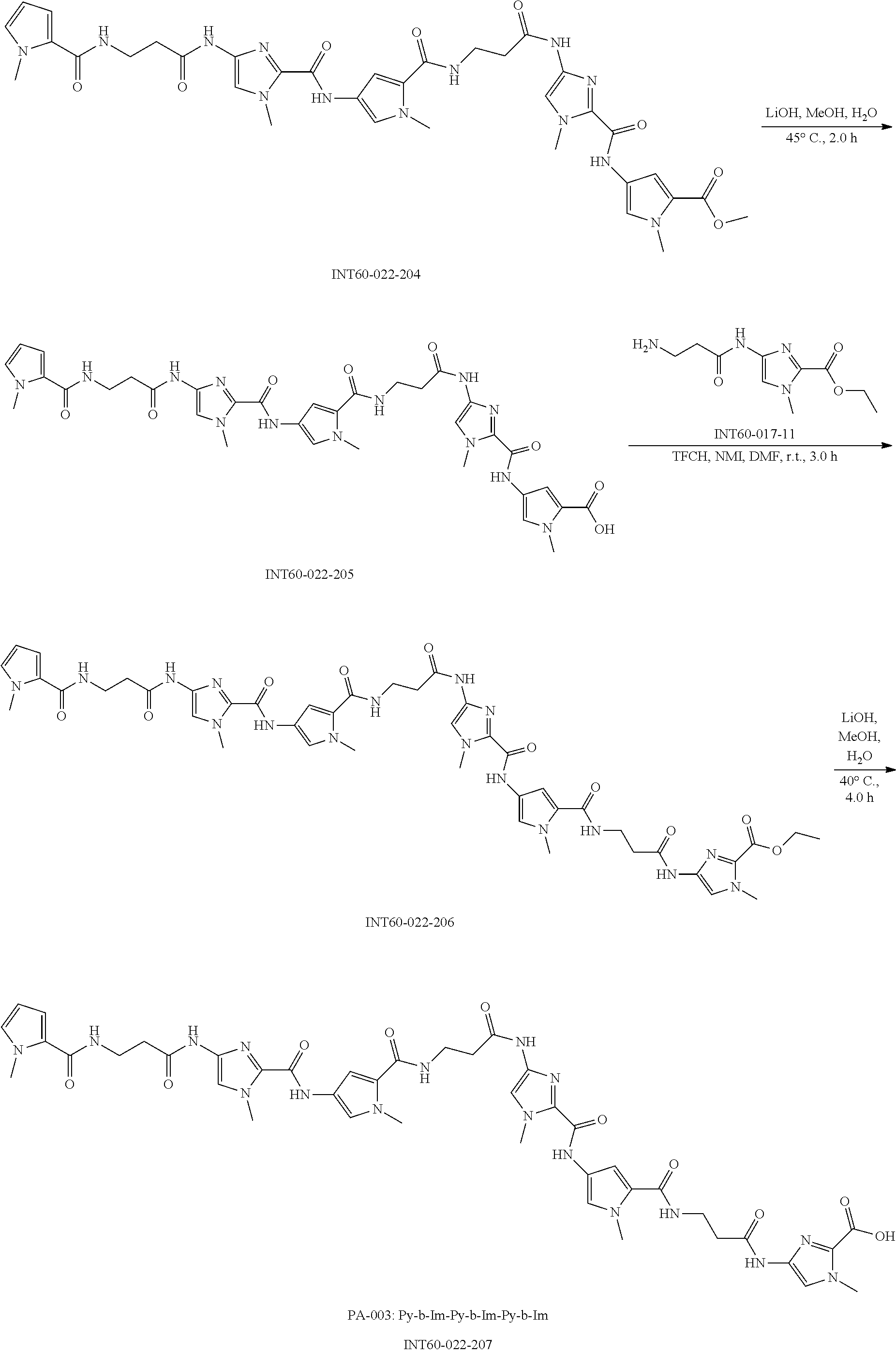 [SMILES: COC(=O)c1cc(NC(=O)c2nc(NC(=O)CCNC(=O)c3cc(NC(=O)c4nc(NC(=O)CCNC(=O)c5cccn5C)cn4C)cn3C)cn2C)cn1C ; Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)O ; CCOC(=O)c1nc(NC(=O)CCN)cn1C ; CCOC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C ; Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)O)n1]

### Step 1: Synthesis of ethyl 4-amino-1-methylimidazole-2-carboxylate (INT60-022-100)

To a solution of ethyl 1-methyl-4-nitroimidazole-2-carboxylate (30.00 g, 150.63 mmol, 1.00 equiv) in EtOH (120.00 mL) and EA (120.00 mL) was added Pd/C (8.01 g, 27% w/w). Then the reaction was stirred for 17.0 h at room temperature under H₂ atmosphere. The solid was filtrated out and the filtrate was concentrated to afford ethyl 4-amino-1-methylimidazole-2-carboxylate (22.30 g, 75.20%) as a yellow solid. LC/MS: mass calcd. For C₇H₁₁N₃O₂: 169.09, found: 170.10 [M+H]⁺.

### Step 2: Synthesis of ethyl 4-[3-[(tert-butoxycarbonyl)amino]propanamido]-1-methylimidazole-2-carboxylate (INT60-017-10)

Into a 500 mL flask was added 3-[(tert-butoxycarbonyl) amino]propanoic acid (22.45 g, 118.65 mmol, 0.90 equiv), DMF (180.00 mL). The mixture was cooled to 0 degrees C., then HATU (75.18 g, 197.71 mmol, 1.50 equiv) and DIEA (51.11 g, 395.43 mmol, 3.00 equiv) were added, the mixture was stirred for 10 mins, then ethyl 4-amino-1-methylimidazole-2-carboxylate (22.30 g, 131.81 mmol, 1.00 equiv) was added in portions. The reaction was stirred at r.t. for 1.0 h. The reaction was quenched with ice water (600 mL), and the solution was stirred for 15 min. The precipitated solids were collected by filtration and washed with water (3×50 mL) dried under vacuum. This resulted in ethyl 4-[3-[(tert-butoxycarbonyl)amino] propanamido]-1-methylimidazole-2-carboxylate (34.50 g, 76.90%) as a light yellow solid. LC/MS: mass calcd. For C₁₅H₂₄N₄O₅: 340.17, found: 341.20 [M+H]⁺.

### Step 3: Synthesis of 4-[3-[(Tert-butoxycarbonyl)amino]propanamido]-1-methylimidazole-2-carboxylic acid (INT60-022-2000)

To a stirred solution of ethyl 4-[3-[(tert-butoxycarbonyl)amino]propanamido]1Methylimidazole-2-carboxylate (34.50 g, 101.36 mmol, 1.00 equiv) in MeOH (200.00 mL) was added LiOH solution (2 M, 202 mL, 4.00 equiv) dropwise at room temperature. The resulting mixture was stirred for 2.0 h at 45° C. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×30 mL), dried under vacuum. 4-[3-[(Tert-butoxycarbonyl)amino]propanamido]-1-methylimidazole-2-carboxylic acid (30.00 g, 94.77%) was obtained as a white solid. LC/MS: mass calcd. For C₁₃H₂₀N₄O₅: 312.14, found: 313.15 [M+H]⁺.

### Step 4: Synthesis of Methyl 4-(4-[3-[(tert-butoxycarbonyl)amino]propanamido]-1-methylimidazole-2-amido)-1-methylpyrrole-2-carboxylate (INT60-022-200)

To a stirred solution of 4-[3-[(tert-butoxycarbonyl)amino]propanamido-1-methylimidazole-2-carboxylic acid (16.00 g, 51.23 mmol, 1.00 equiv) in CH3CN (150.00 mL) was added TCFH (21.56 g, 76.84 mmol, 1.50 equiv), NMI (12.62 g, 153.69 mmol, 3.00 equiv) and methyl 4-amino-1-methylpyrrole-2-carboxylate hydrochloride (10.74 g, 56.34 mmol, 1.10 equiv) in portions at 0 degrees C. The resulting mixture was stirred for 2.0 h at room temperature. The precipitated solids were collected by filtration and washed by CH3CN (3×20 mL), dried under vacuum. Methyl 4-(4-[3-[(tert-butoxycarbonyl)amino]propanamido]-1-methylimidazole-2-amido)-1-methylpyrrole-2-carboxylate (19.00 g, 82.70%) was obtained as a white solid. LC/MS: mass calcd. For C₂₀H₂₈N₆O₆: 448.21, found: 449.25 [M+H]⁺.

### Step 5: Synthesis of methyl 4-[4-(3-aminopropanamido)-1-methylimidazole-2-amido]-1-methylpyrrole-2-carboxylate hydrochloride (INT60-022-201)

A solution of methyl 4-(4-[3-[(tert-butoxycarbonyl)amino]propanamido]-1-methylimidazole-2-amido)-1-methylpyrrole-2-carboxylate (19.00 g, 42.37 mmol, 1.00 equiv) in HCl/1,4-dioxane (4M, 200.00 mL) was stirred for 2 h at room temperature. The resulting mixture was concentrated under vacuum. Methyl 4-[4-(3-aminopropanamido)-1-methylimidazole-2-amido]-1-methylpyrrole-2-carboxylate hydrochloride (19.00 g crude) was obtained as a yellow solid. LC/MS: mass calcd. For C₁₅H₂₁ClN₆O₄: 348.15, found: 349.05 [M+H]⁺.

### Step 6: Synthesis of ethyl 4-(3-aminopropanamido)-1-methyl-1H-imidazole-2-carboxylate (INT60-017-11)

The procedure was the same as methyl 4-[4-(3-aminopropanamido)-1-methylimidazole-2-amido]-1-methylpyrrole-2-carboxylate hydrochloride (INT60-017-11). 2.00 g of ethyl 4-[3-[(tert-butoxycarbonyl) amino]propanamido]-1-methylimidazole-2-carboxylate was used, 2.00 g crude of desired product was obtained as off-white solid. LC/MS: mass calcd. For C₁₀H₁₆N₄O₃: 240.12, found: 241.10 [M+H]⁺+.

### Step 7: Synthesis of methyl 1-methyl-4-(1-methyl-4-[3-[(1-methylpyrrol-2-yl) formamido]propanamido]imidazole-2-amido)pyrrole-2-carboxylate (INT60-022-202)

To a solution of 1-methylpyrrole-2-carboxylic acid (600.00 mg, 4.80 mmol, 1.00 equiv) in CH₃CN (20.00 mL) was added NMI (1.22 g, 14.87 mmol, 3.10 equiv), TCFH (1.48 g, 5.28 mmol, 1.10 equiv) and methyl 4-[4-(3-aminopropanamido)-1-methylimidazole-2-amido]-1-methylpyrrole-2-carboxylate (2004.53 mg, 5.75 mmol, 1.20 equiv). The mixture was stirred at r.t. for 2.0 h. The solvent was removed and the residue was purified by reverse phase column under the condition: column, C18 column, MeCN/water (0.05% TFA), 5% to 50% gradient in 100 min; detector, 254 nm. The fractions were combined and concentrated. 1.30 g of desired product was obtained ad white solid (56% yield). LC/MS: mass calcd. For C₂₁H₂₅N₇O₅: 455.19, found: 456.30 [M+H]⁺.

### Step 8: Synthesis of 1-methyl-4-(1-methyl-4-[3-[(1-methylpyrrol-2-yl)formamido] propanamido]imidazole-2-amido)pyrrole-2-carboxylic acid (INT60-022-203)

The procedure was the same as 4-[3-[(tert-butoxycarbonyl)amino]propanamido]-1-methylimidazole-2-carboxylic acid (INT60-022-2000). 2.00 g of methyl 1-methyl-4-(1-methyl-4-[3-[(1-methylpyrrol-2-yl)formamido]propanamido]imidazole-2-amido)pyrrole-2-carboxylate was used, 1.90 g of desired product was obtained as white solid (92.00% yield). LC/MS: mass calcd. For C₂₀H₂₃N₇O₅: 441.18, found: 442.25 [M+H]⁺.

### Step 9: Synthesis of methyl 1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrole-2-carboxylate (INT60-022-204)

The procedure was the same as methyl 4-(4-[3-[(tert-butoxycarbonyl)amino] propanamido]-1-methylimidazole-2-amido)-1-methylpyrrole-2-carboxylate (INT60-022-200), but the filtrate was concentrated and purified by reverse phase column. 1.90 g of 1-methyl-4-(1-methyl-4-[3-[(1-methylpyrrol-2-yl)formamido] propanamido]imidazole-2-amido)pyrrole-2-carboxylic acid was used, 2.70 g of desired product was obtained as whit solid (71.00% yield). LC/MS: mass calcd. For C₃₅H₄₁N₁₃O₈: 771.32, found: 772.35 [M+H]⁺.

### Step 10: Synthesis of 1-methyl-4-[1-methyl-4-(3-[[1-methyl-4-(1-methyl-4-[3-[(1-methylpyrrol-2-1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrole-2-carboxylic acid (INT60-022-205)

The procedure was the same as 4-[3-[(tert-butoxycarbonyl)amino]propanamido]-1-methylimidazole-2-carboxylic acid (INT60-022-2000). 2.70 g of methyl 1-methyl-4-[1-methyl-4-(3-[[1-methyl-4-(1-methyl-4-[3-[(1-methylpyrrol-2-yl) formamido]propanamido]imidazole-2-amido)pyrrol-2-yl]formamido]propanamido)imidazole-2-amido]pyrrole-2-carboxylate was used, 2.80 g of desired product was obtained as white solid. (78.00% yield). LC/MS: mass calcd. For C₃₄H₃₉N₁₃O₈: 757.30, found: 758.50 [M+H]⁺.

### Step 11: Synthesis of ethyl 1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-carboxylate (INT60-022-206)

To a solution of 1-methyl-4-[1-methyl-4-(3-[[1-methyl-4-(1-methyl-4-[3-[(1-methylpyrrol-2-1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido] propanamido)imidazole-2-amido]pyrrole-2-carboxylic acid (2.90 g, 3.83 mmol, 1.00 equiv) in DMF (25.00 mL) was added NMI (3.20 g, 39.04 mmol, 10.20 equiv), TCFH (1.18 g, 4.21 mmol, 1.10 equiv) and ethyl 4-(3-aminopropanamido)-1-methylimidazole-2-carboxylate (1.16 g, 4.21 mmol, 1.10 equiv). Then reaction was stirred at r.t. for 3.0 h. The mixture was poured into ice water, the solid was filtered out, then it was purified by silica gel column chromatography (DCM/MeOH=10:1). 2.5 g of desired product was obtained as white solid (66.00% yield). LC/MS: mass calcd. For C₄₄H₅₃N₁₇O₁₀: 979.42, found: 980.80 [M+H]⁺.

### Step 12: Synthesis of 1-methyl-4-(3-(1-methyl-4-(1-methyl-4-(3-(1-methyl-4-(1-methyl-4-(3-(1-methyl-1H-pyrrole-2-carboxamido)propanamido)-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylic acid (PA-003)

The procedure was the same as 4-[3-[(tert-butoxycarbonyl)amino] propanamido]-1-methylimidazole-2-carboxylic acid (INT60-022-2000), but the reaction temperature was 40° C. and reaction time is 4.0 h. 2.50 g of ethyl 1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-carboxylate was used, 1.90 g of desired product was observed as white solid (78.00% yield). LC/MS: mass calcd. For C₄₂H₄₉N₁₇O₁₀: 951.38, found: 952.65 [M+H]⁺.

### Example 26. Synthesis of PA-004

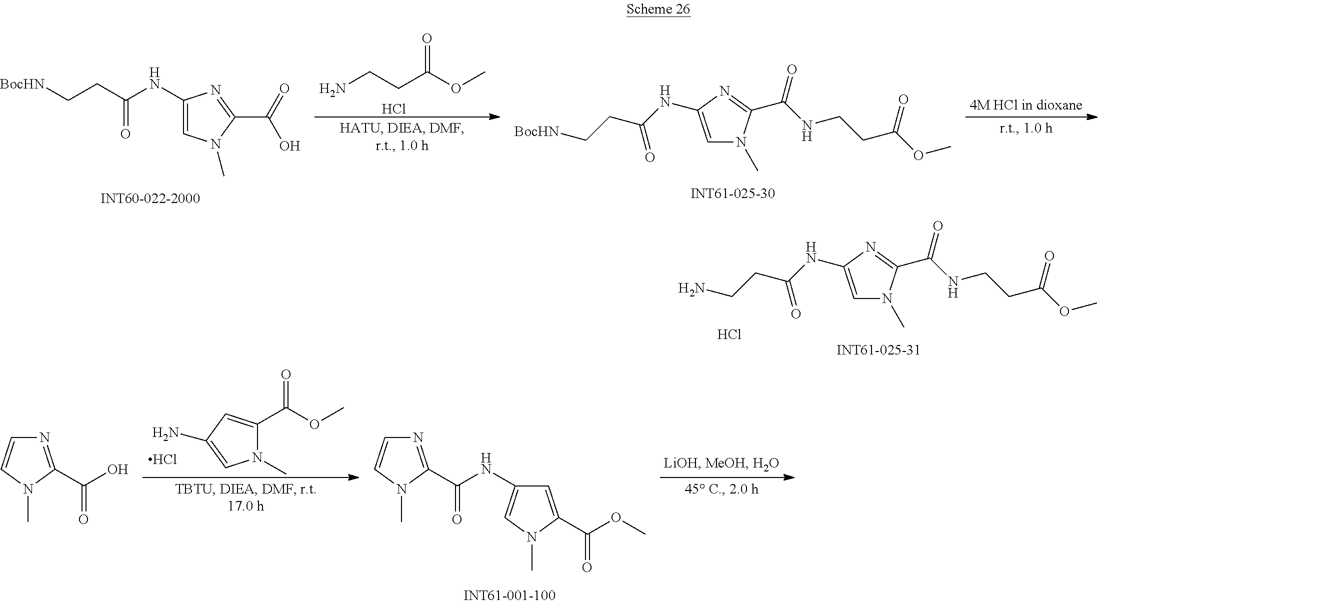

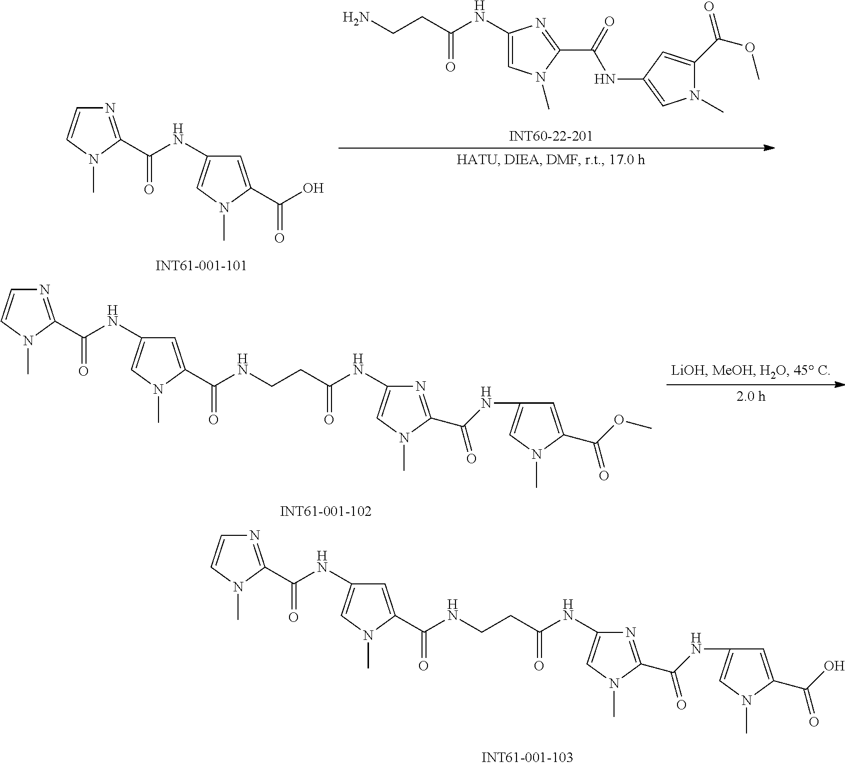 [SMILES: Cn1cc(NC(=O)c2nccn2C)cc1C(=O)O ; COC(=O)c1cc(NC(=O)c2nc(NC(=O)CCN)cn2C)cn1C ; COC(=O)c1cc(NC(=O)c2nc(NC(=O)CCNC(=O)c3cc(NC(=O)c4nccn4C)cn3C)cn2C)cn1C ; Cn1cc(NC(=O)c2nc(NC(=O)CCNC(=O)c3cc(NC(=O)c4nccn4C)cn3C)cn2C)cc1C(=O)O]

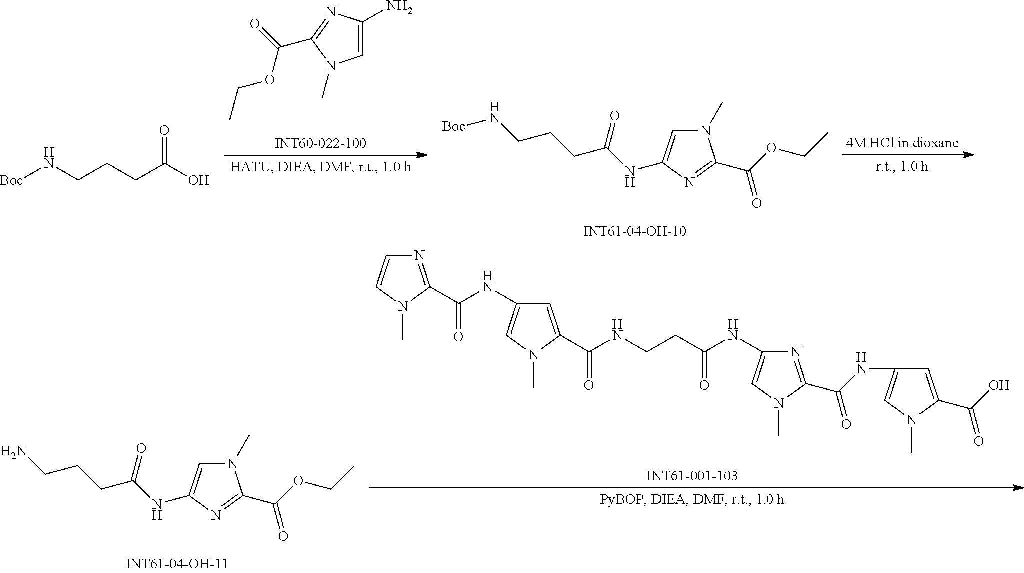 [SMILES: CC(C)(C)OC(=O)NCCCC(=O)O ; CCOC(=O)c1nc(N)cn1C ; CCOC(=O)c1nc(NC(=O)CCCNC(=O)OC(C)(C)C)cn1C ; CCOC(=O)c1nc(NC(=O)CCCN)cn1C ; Cn1cc(NC(=O)c2nc(NC(=O)CCNC(=O)c3cc(NC(=O)c4nccn4C)cn3C)cn2C)cc1C(=O)O]

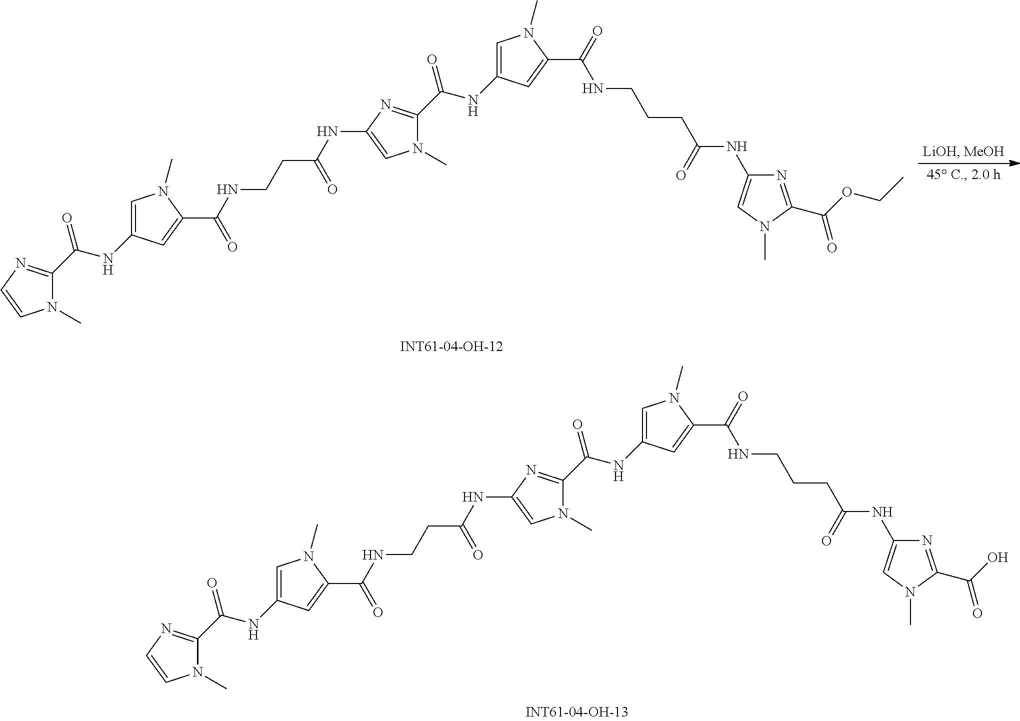 [SMILES: CCOC(=O)c1nc(NC(=O)CCCNC(=O)c2cc(NC(=O)c3nc(NC(=O)CCNC(=O)c4cc(NC(=O)c5nccn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1]

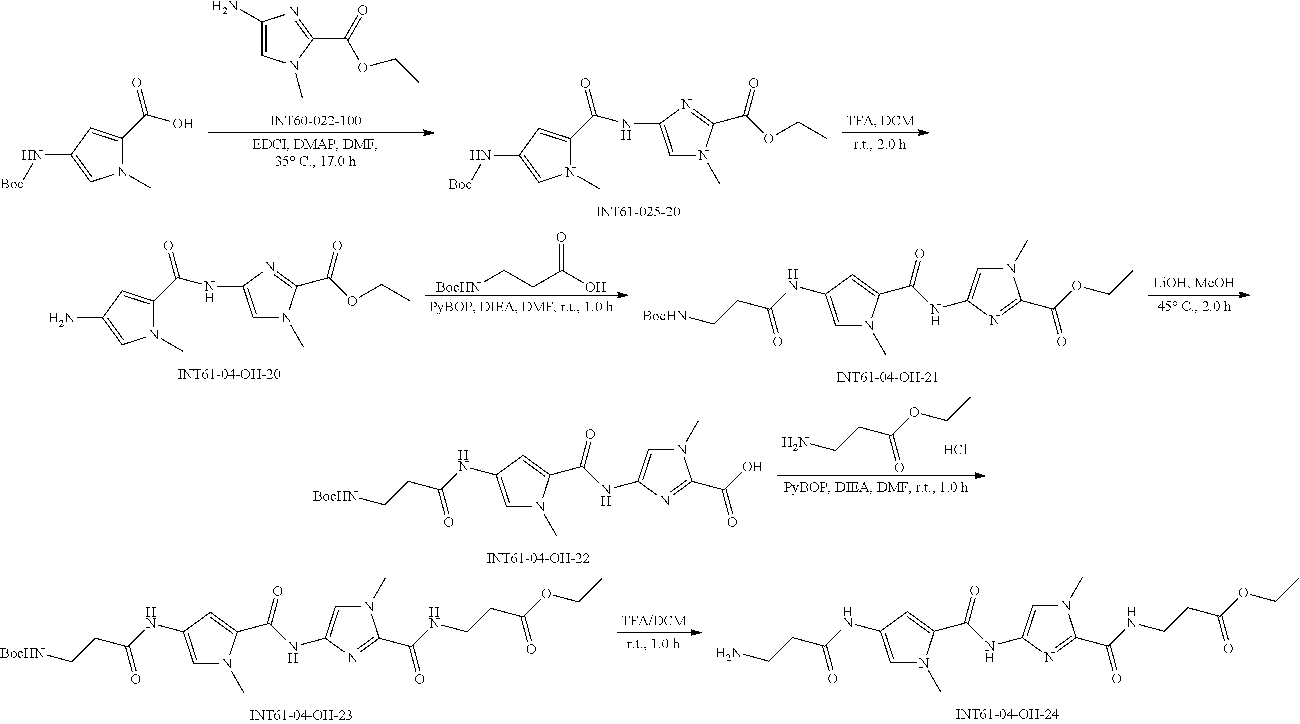

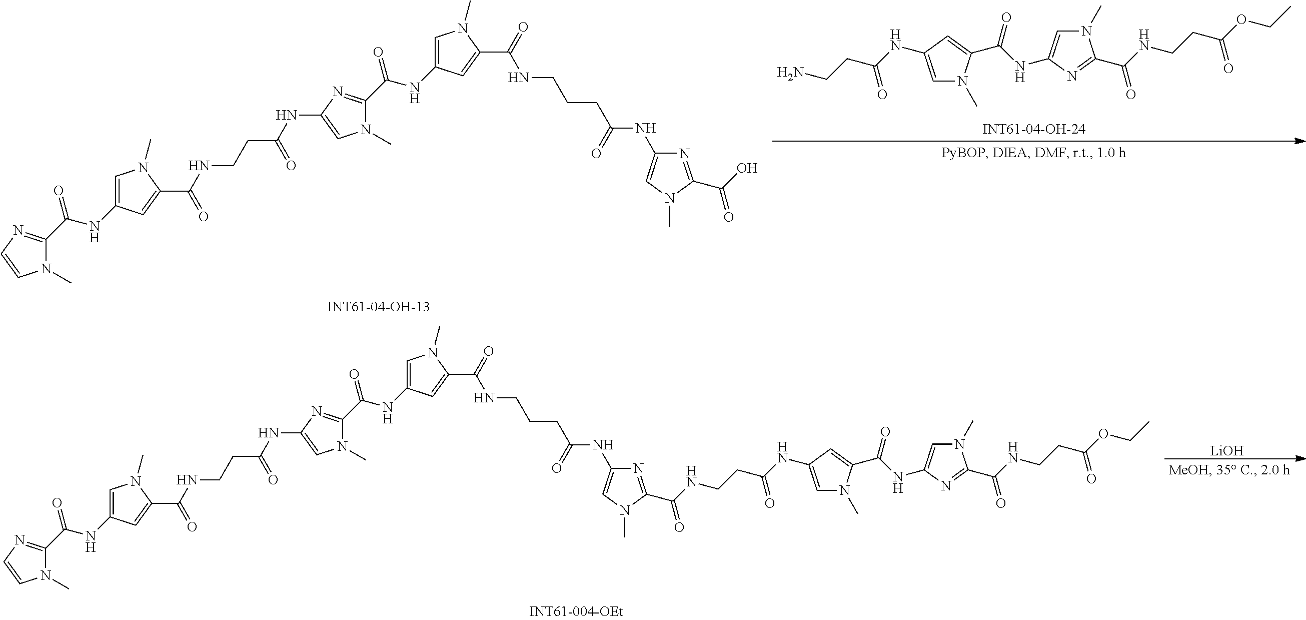 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1 ; CCOC(=O)CCNC(=O)c1nc(NC(=O)c2cc(NC(=O)CCN)cn2C)cn1C ; CCOC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C ; "LiOH"]

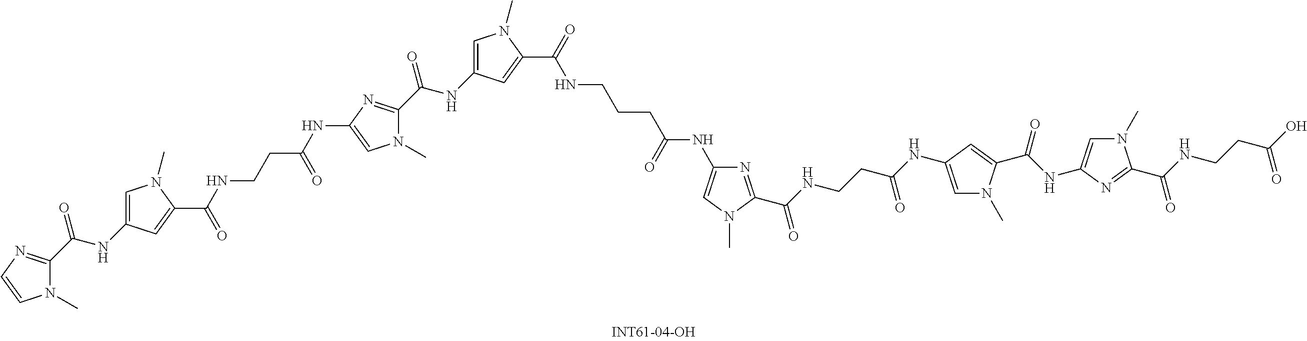 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

### Step 1: Synthesis of methyl 3-[(4-[3-[(tert-butoxycarbonyl)amino] propanamido]-1-methylimidazol-2-yl)formamido]propanoate (INT61-025-30)

Into a 1000 ml flask was added 4-[3-[(tert-butoxycarbonyl)amino] propanamido]-1-methylimidazole-2-carboxylic acid (11.00 g, 35.22 mmol, 1.00 equiv), DMF (300.00 mL), the mixture was cooled to 0 degrees C., then HATU (20.09 g, 52.83 mmol, 1.50 equiv), DIEA (18.21 g, 140.88 mmol, 4.00 equiv) was added dropwise, the mixture was stirred for 10 mins, methyl 3-aminopropanoate (3.63 g, 35.22 mmol, 1.00 equiv) was added in portions. The reaction was stirred at r.t. for 1.0 h. The reaction mixture was poured into water/ice (600 mL), the solid was filtered out and dried under vacuum. The aqueous phase was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue was purified by silica gel column, eluted with pure EA. The fractions were combined and concentrated. Methyl 3-[(4-[3-[(tert-butoxycarbonyl)amino]propanamido]-1-methylimidazol-2-yl)formamido]propanoate (13.00 g, 87.95%) was obtained as a yellow solid. LC/MS: mass calcd. For C₁₇H₂₇N₅O₆: 397.20, found: 398.20 [M+H]⁺.

### Step 2: Synthesis of methyl 3-[[4-(3-aminopropanamido)-1-methylimidazol-2-yl] formamido]propanoate hydrochloride

The procedure was the same as methyl 4-[4-(3-aminopropanamido)-1-methylimidazole-2-amido]-1-methylpyrrole-2-carboxylate hydrochloride (INT60-017-11), but the reaction time was 1.0 h. 11.00 g of methyl 3-[(4-[3-[(tert-butoxycarbonyl)amino]propanamido]-1-methylimidazol-2-yl)formamido]propanoate was used, 11.00 g crude of desired product was obtained as yellow oil. LC/MS: mass calcd. For C₁₂H₁₉N₅O₄: 297.14, found: 298.20 [M+H]⁺.

### Step 3: Synthesis of Methyl 1-methyl-4-(1-methylimidazole-2-amido)pyrrole-2-carboxylate (INT61-001-100)

To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv) and DIEA (30.74 g, 237.88 mmol, 3.00 equiv) in portions at 0 degrees C. The resulting mixture was stirred for 17.0 h at room temperature. The reaction was poured into water/Ice (450 mL). The precipitated solids were collected by filtration and washed with H₂O (3×50 mL), dried under vacuum. Methyl 1-methyl-4-(1-methylimidazole-2-amido)pyrrole-2-carboxylate (16.5 g, 78.37%) was obtained as a white solid. LC/MS: mass calcd. For C₁₂H₁₄N₄O₃: 262.11, found: 263.15 [M+H]⁺.

### Step 4: Synthesis of 1-methyl-4-(1-methylimidazole-2-amido)pyrrole-2-carboxylic acid (INT61-001-101)

The procedure was the same as 4-[3-[(tert-butoxycarbonyl)amino] propanamido]-1-methylimidazole-2-carboxylic acid (INT60-022-2000). 16.50 g of methyl 1-methyl-4-(1-methylimidazole-2-amido)pyrrole-2-carboxylate was used, 12.00 g of 1-methyl-4-(1-methylimidazole-2-amido)pyrrole-2-carboxylic acid (76.84% yield) was obtained as white solid. LC/MS: mass calcd. For C₁₁H₁₂N₄O₃: 248.09, found: 249.10 [M+H]⁺.

### Step 5: Synthesis of methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate (INT61-001-102)

The procedure was the same as ethyl 3-[(4-[3-[(tert-butoxycarbonyl)amino] propanamido]-1-methylimidazol-2-yl)formamido]propanoate (INT60-017-10). 9.00 g of 1-methyl-4-(1-methylimidazole-2-amido)pyrrole-2-carboxylic acid was used, 14.00 g of desired product (63.54% yield) was obtained yellow solid. LC/MS: mass calcd. For C₂₆H₃₀N₁₀O₆: 578.23, found: 579.10 [M+H]⁺.

### Step 6: Synthesis of 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic acid (INT61-001-103)

The procedure was the same as 4-[3-[(Tert-butoxycarbonyl)amino] propanamido]-1-methylimidazole-2-carboxylic acid (INT60-022-2000). 14.00 g of methyl 1-methyl-4-[1-methyl-4-(3-[[1-methyl-4-(1-methylimidazole-2-amido) pyrrol-2-yl]formamido]propanamido)imidazole-2-amido]pyrrole-2-yl]formamidocarboxylate was used, 12.00 g of desired product (81.49% yield) was obtained as yellow solid. LC/MS: mass calcd. For C₂₅H₂₈N₁₀O₆: 564.22, found: 565.15[M+H]⁺.

### Step 7: Synthesis of ethyl 4-{4-[(tert-butoxycarbonyl)amino]butanamido}-1-methylimidazole-2-carboxylate (INT61-04-OH-10)

The procedure was the same as ethyl 4-[3-[(tert-butoxycarbonyl)amino]propanamido]-1-methylimidazole-2-carboxylate (INT60-017-10). 7.80 g of 4-[(tert-butoxycarbonyl)amino]butanoic acid was obtained, 11.00 g of desired product was obtained as little pink solid (80.70% yield). LC/MS: mass calcd. For C₁₆H₂₆N₄O₅: 354.19, found: 355.15[M+H]⁺.

### Step 8: Synthesis of ethyl 4-(4-aminobutanamido)-1-methylimidazole-2-carboxylate (INT61-04-OH-11)

The procedure was the same as methyl 4-[4-(3-aminopropanamido)-1-methylimidazole-2-amido]-1-methylpyrrole-2-carboxylate hydrochloride (INT60-017-11). 9.40 g of ethyl 4-{4-[(tert-butoxycarbonyl)amino]butanamido}-1-methylimidazole-2-carboxylate was used, 6.20 g of desired product was obtained as a white solid (90.89% yield). LCMS: mass calcd. For C₁₁H₁₈N₄O₃: 254.14, found: 255.15[M+H]⁺.

### Step 9: Synthesis of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (INT61-04-OH-12)

To a stirred solution of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid (18.20 g, 32.24 mmol, 1.00 equiv) in DMF (250.00 mL) was added DIEA (12.50 g, 96.71 mmol, 3.00 equiv), ethyl 4-(4-aminobutanamido)-1-methylimidazole-2-carboxylate (9.02 g, 35.46 mmol, 1.10 equiv) and PyBOP (20.13 g, 38.68 mmol, 1.20 equiv) at 0 degrees C. The resulting mixture was stirred for 1.0 h at room temperature. The reaction was poured into ice/water (800 mL). The precipitated solids were collected by filtration and washed with H₂O (3×200 mL), dried under vacuum. 24.70 g of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate was obtained as a yellow solid (95.74% yield). LC/MS: mass calcd. For C₃₆H₄₄N₁₄O₈: 800.35, found: 801.30[M+H]⁺.

### Step 10: Synthesis of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid (INT61-04-OH-13)

The procedure was the same as 4-[3-[(tert-butoxycarbonyl)amino]propanamido]-1-methylimidazole-2-carboxylic acid (INT60-022-2000). 24.00 g of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate was used, 23.10 g of desired product was obtained as a yellow solid (99.36% yield). LC/MS: mass calcd. For C₃₄H₄₀N₁₄O₈: 772.32, found: 773.30[M+H]⁺.

### Step 11: Synthesis of ethyl 4-[4-[(tert-butoxycarbonyl)amino]-1-methylpyrrole-2-amido]-1-methylimidazole-2-carboxylate (INT61-025-20)

To a stirred solution of 4-[(tert-butoxycarbonyl)amino]-1-methylpyrrole-2-carboxylic acid (11.50 g, 47.87 mmol, 1.00 equiv) in DMF (200.00 mL) was added EDCI (22.94 g, 119.66 mmol, 2.50 equiv), ethyl 4-amino-1-methylimidazole-2-carboxylate (8.10 g, 47.87 mmol, 1.00 equiv) and DMAP (14.62 g, 119.66 mmol, 2.50 equiv) at 0 degrees C. The resulting mixture was stirred for 17.0 h at 35° C. After reaction, the reaction was poured into 500 mL ice/water. The precipitated solids were collected by filtration and washed with water (3×50 mL), dried under vacuum. This resulted in ethyl 4-{4-[(tert-butoxycarbonyl)amino]-1-methylpyrrole-2-amido}-1-methylimidazole-2-carboxylate (16.00 g, 85.48% yield) as a light yellow solid. LC/MS: mass calcd. For C₁₈H₂₅N₅O₅: 391.19, found: 392.30 [M+H]⁺.

### Step 12: Synthesis of ethyl 4-(4-amino-1-methylpyrrole-2-amido)-1-methylimidazole-2-carboxylate (INT61-04-OH-20)

To a stirred solution of ethyl 4-{4-[(tert-butoxycarbonyl)amino]-1-methylpyrrole-2-amido}-1-methylimidazole-2-carboxylate (16.00 g, 40.88 mmol, 1.00 equiv) in DCM (135.00 mL) were added and TFA (45.00 mL) dropwise at room temperature. The resulting mixture was stirred for 2.0 h at room temperature. The resulting mixture was concentrated under vacuum. The residue brown oil was diluted with Et₂O (200 mL). The precipitated solids were collected by filtration and washed with Et₂O (2×100 mL). The resulting solid was dried under vacuum. This resulted in ethyl 4-(4-amino-1-methylpyrrole-2-amido)-1-methylimidazole-2-carboxylate (16.00 g, crude) as a brown solid. LC/MS: mass calcd. For C₁₃H₁₇N₅O₃: 291.13, found: 292.15[M+H]⁺.

### Step 13: Synthesis of ethyl 4-(4-{3-[(tert-butoxycarbonyl)amino]propanamido}-1-methylpyrrole-2-amido)-1-methylimidazole-2-carboxylate (INT61-04-OH-21)

A solution of ethyl 4-(4-amino-1-methylpyrrole-2-amido)-1-methylimidazole-2-carboxylate (12.00 g, 41.19 mmol, 1.00 equiv) and 3-[(tert-butoxycarbonyl)amino] propanoic acid (7.50 g, 39.64 mmol, 0.96 equiv), PyBOP (22.00 g, 42.28 mmol, 1.03 equiv), DIEA (45.00 g, 348.18 mmol, 8.45 equiv) in DMF (120.00 mL) was stirred for 1.0 h at room temperature. The reaction was poured into ice water (400 mL), and the mixture was stirred for 15 min. The precipitated solids were collected by filtration and washed with water (3×150 mL) and dried under vacuum. The aqueous phase was extracted by EA (3×150 mL), the combined organic phases were combined and washed by H₂O (200 mL), dried over anhydrous Na₂SO₄. The solid was filtered out and the filtrate was concentrated. The residue was purified by silica gel column chromatography, eluted with PE/EA (1:8). Totally 17.00 g of ethyl 4-(4-{3-[(tert-butoxycarbonyl)amino]propanamido}-1-methylpyrrole-2-amido)-1-methylimidazole-2-carboxylate was obtained as a yellow solid (89.28% yield). LC/MS: mass calcd. For C₂₁H₃₀N₆O₆: 462.22, found: 463.35[M+H]⁺.

### Step 14: Synthesis of 4-(4-{3-[(tert-butoxycarbonyl)amino]propanamido}-1-methylpyrrole-2-amido)-1-methylimidazole-2-carboxylic acid (INT61-04-OH-22)

The procedure was the same as 4-[3-[(tert-butoxycarbonyl)amino]propanamido]-1-methylimidazole-2-carboxylic acid (INT60-022-2000). 12.00 g of ethyl 4-(4-{3-[(tert-butoxycarbonyl)amino]propanamido}-1-methylpyrrole-2-amido)-1-methylimidazole-2-carboxylate was used, 10.00 g of desired product was obtained as white solid (88.81% yield). LC/MS: mass calcd. For C₁₉H₂₆N₆O₆: 434.19, found: 435.25[M+H]⁺.

### Step 15: Synthesis of ethyl 3-{[4-(4-{3-[(tert-butoxycarbonyl)amino]propanamido}-1-methylpyrrole-2-amido)-1-methylimidazol-2-yl]formamido}propanoate (INT61-04-OH-23)

A solution of 4-(4-{3-[(tert-butoxycarbonyl)amino]propanamido}-1-methylpyrrole-2-amido)-1-methylimidazole-2-carboxylic acid (10.00 g, 23.02 mmol, 1.00 equiv) and $-alanine ethyl ester hydrochloride (4.90 g, 31.90 mmol, 1.39 equiv), PyBOP (12.50 g, 24.02 mmol, 1.04 equiv), DIEA (9.00 g, 69.64 mmol, 3.03 equiv) in DMF (120.00 mL) was stirred for 1.0 h at room temperature. The reaction was quenched by the addition of water (500 mL) at room temperature. The resulting mixture was extracted with EtOAc (3×400 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (1:8) to afford ethyl 3-{[4-(4-{3-[(tert-butoxycarbonyl)amino]propanamido}-1-methylpyrrole-2-amido)-1-methylimidazol-2-yl]formamido}propanoate (12.00 g, 93.80%) as a yellow solid. LC/MS: mass calcd. For C₂₄H₃₅N₇O₇: 533.26, found: 534.30[M+H]⁺.

### Step 16: Synthesis of ethyl 3-({4-[4-(3-aminopropanamido)-1-methylpyrrole-2-amido]-1-methylimidazol-2-yl}formamido)propanoate (INT61-04-OH-24)

The procedure was the same as ethyl 4-(4-amino-1-methylpyrrole-2-amido)-1-methylimidazole-2-carboxylate (INT61-04-OH-20). 12.00 g of ethyl 3-{[4-(4-{3-[(tert-butoxycarbonyl)amino]propanamido}-1-methylpyrrole-2-amido)-1-methylimidazol-2-yl]formamido}propanoate was used, 12.00 g crude of desired product was obtained as white solid. LC/MS: mass calcd. For C₁₉H₂₇N₇O₅: 433.21, found: 434.25[M+H]⁺.

### Step 17: Synthesis of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate (INT61-004-OEt)

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (INT61-04-OH-12). 10.00 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used, 13.60 g of desired product was obtained as yellow solid (88.61% yield). Some pure product was obtained as light yellow solid after purification by Prep-HPLC. HRMS: mass calcd. For C₅₃H₆₅N₂₁O₁₂: 1187.5122, found: 1188.5153[M+H]⁺.

### Step 18: Synthesis of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (PA-004-OH)

The procedure was the same as 4-[3-[(tert-butoxycarbonyl)amino]propanamido]-1-methylimidazole-2-carboxylic acid (INT60-022-2000), but the reaction temperature was 35° C. 10.60 g of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate was used, 10.00 g crude of desired product was obtained as yellow solid. LC/MS: mass calcd. For C₅₁H₆₁N₂₁O₁₂: 1159.48, found: 581.25[M/2+H]⁺.

### Example 27. Synthesis of PA-023

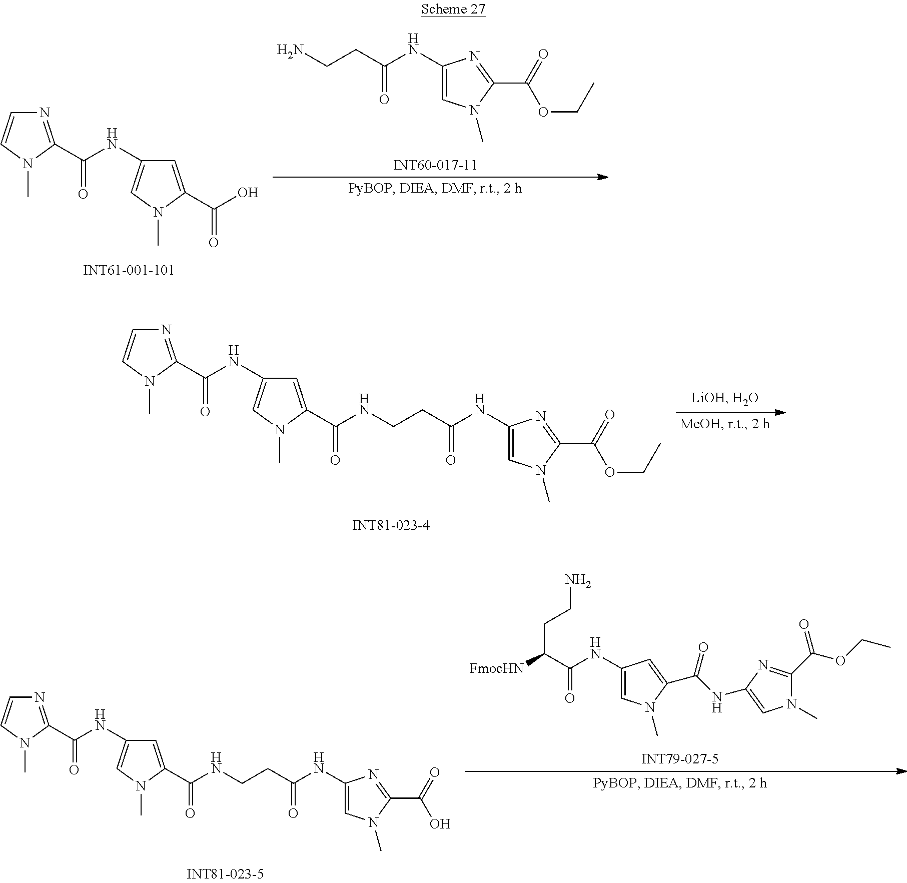

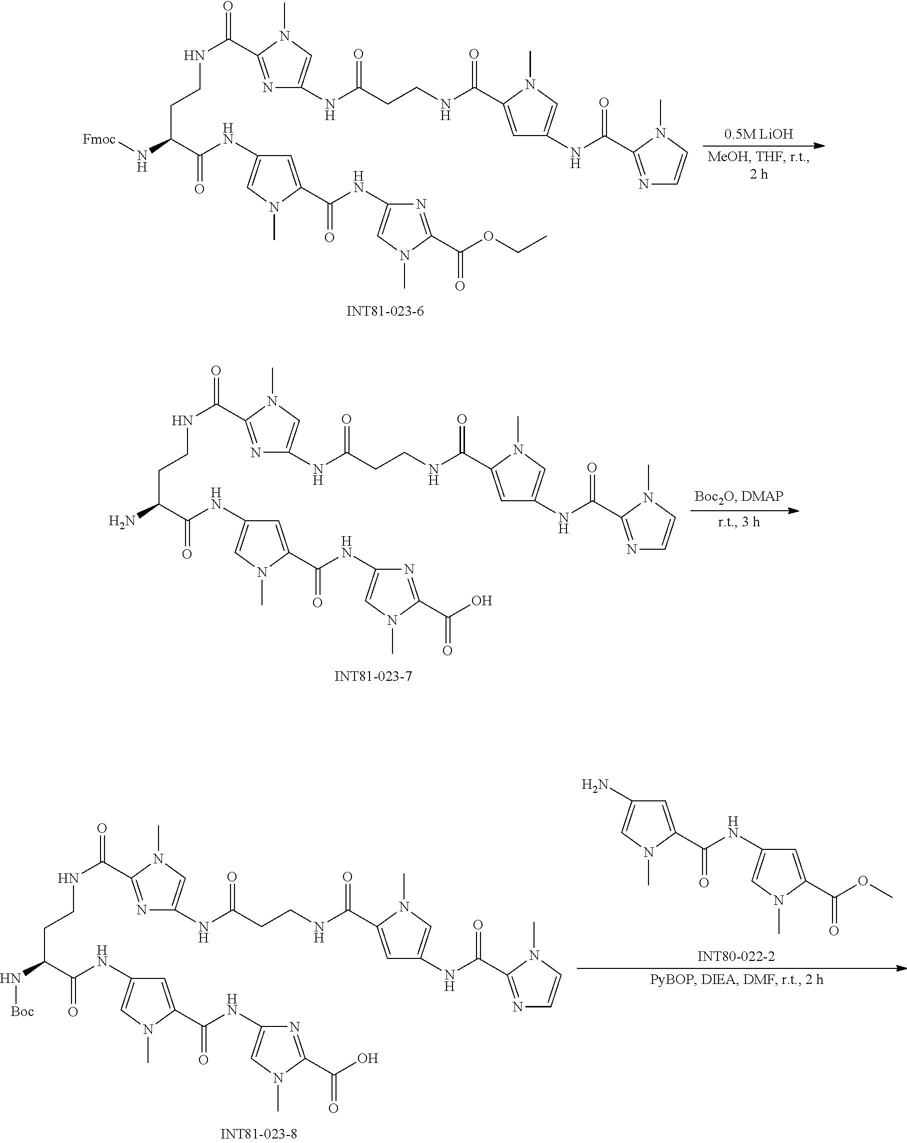

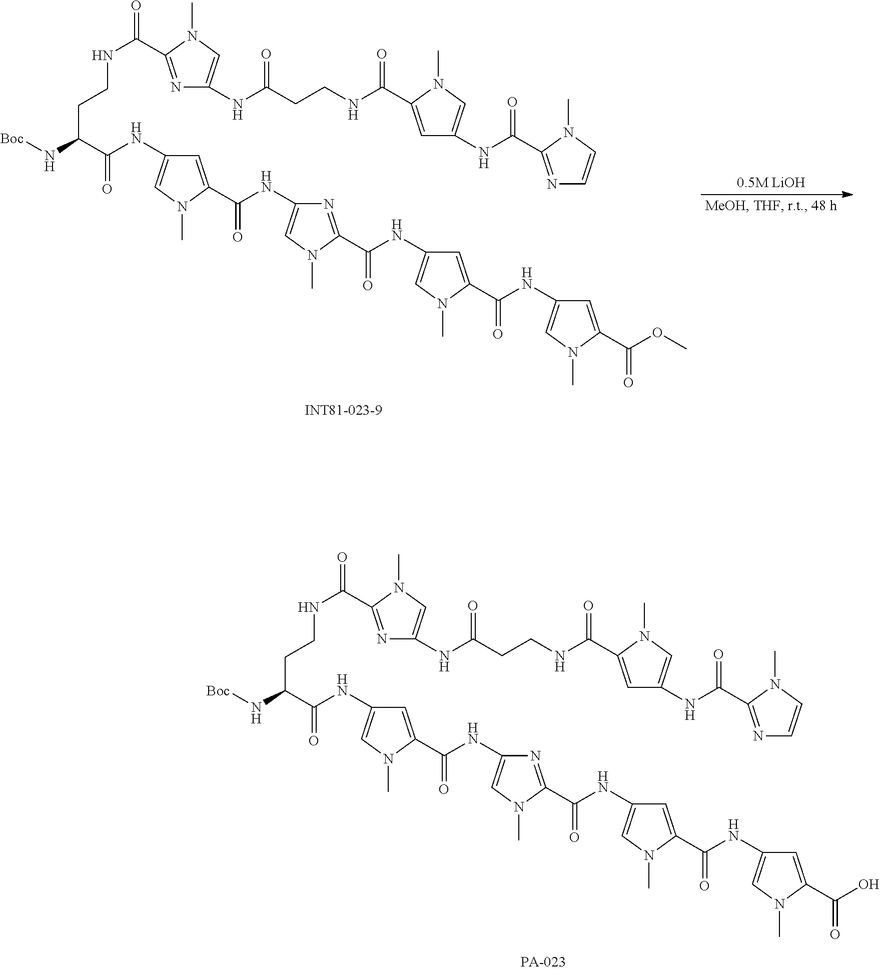 [SMILES: COC(=O)c1cc(NC(=O)c2cc(NC(=O)c3nc(NC(=O)c4cc(NC(=O)[C@H](CCNC(=O)c5nc(NC(=O)CCNC(=O)c6cc(NC(=O)c7nccn7C)cn6C)cn5C)NC(=O)OC(C)(C)C)cn4C)cn3C)cn2C)cn1C ; Cn1cc(NC(=O)c2cc(NC(=O)c3nc(NC(=O)c4cc(NC(=O)[C@H](CCNC(=O)c5nc(NC(=O)CCNC(=O)c6cc(NC(=O)c7nccn7C)cn6C)cn5C)NC(=O)OC(C)(C)C)cn4C)cn3C)cn2C)cc1C(=O)O]

### Step 1: Synthesis of ethyl 1-methyl-4-(3-{[1-methyl-4-(1-methylimidazole-2-amido)pyrrol-2-yl]formamido}propanamido)imidazole-2-carboxylate (INT81-023-4)

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (INT61-04-OH-12), but the reaction time was 2.0 h. 1.50 g of ethyl 4-(3-aminopropanamido)-1-methylimidazole-2-carboxylate was used, 2.00 g of desired product was obtained as an off-white solid (68.09% yield). LC/MS: mass calcd. For C₂₁H₂₆N₈O₅: 470.20, found: 471.40 [M+H]⁺.

### Step 2: Synthesis of 1-methyl-4-(3-{[1-methyl-4-(1-methylimidazole-2-amido) pyrrol-2-yl]formamido}propanamido)imidazole-2-carboxylic acid (INT81-023-5)

The procedure was the same as 4-[3-[(tert-butoxycarbonyl)amino]propanamido]-1-methylimidazole-2-carboxylic acid (INT60-022-2000), but the reaction temperature was room temp. (r.t.), the reaction time was 2.0 h. 2.00 g of ethyl 1-methyl-4-(3-{[1-methyl-4-(1-methylimidazole-2-amido)pyrrol-2-yl]formamido} propanamido)imidazole-2-carboxylate was used, 1.80 g of 1-methyl-4-(3-{[1-methyl-4-(1-methylimidazole-2-amido)pyrrol-2-yl]formamido}propanamido)imidazole-2-carboxylic acid was obtained as an off-white solid (95.71% yield). LC/MS: mass calcd. For C₁₉H₂₂N₈O₅: 442.17, found: 443.10 [M+H]⁺.

### Step 3: Synthesis of ethyl 4-{4-[(2S)-2-{[(9H-fluoren-9-ylmethoxy)carbonyl] amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate (INT81-023-6)

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (INT61-04-OH-12), but the reaction time was 2.0 h. 1.60 g of ethyl 4-{4-[(2S)-4-amino-2-{[(9H-fluoren-9-ylmethoxy)carbonyl]amino}butanamido]-1-methylpyrrole-2-amido}-1-methylimidazole-2-carboxylate was used, 1.90 g of desired product was obtained as a light yellow solid (70.20% yield). LC/MS: mass calcd. For C₅₁H₅₅N₁₅O₁₀: 1037.43, found: 1038.45 [M+H]⁺.

### Step 4: Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid (INT81-023-7)

A mixture of ethyl 4-{4-[(2S)-2-{[(9H-fluoren-9-ylmethoxy)carbonyl]amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate (1.90 g, 1.83 mmol, 1.00 equiv) and LiOH (0.22 g, 9.15 mmol, 5.00 equiv) in MeOH (5.00 mL), THF (15.00 mL) and H₂O (18.30 mL) was stirred for 2.0 h at room temperature. The resulting mixture was used in the next step directly without further purification. LC/MS: mass calcd. For C₃₄H₄₁N₁₅O₈: 787.33, found: 788.40 [M+H]⁺.

### Step 5: Synthesis of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (INT81-023-8)

The mixture of 4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.40 g, 1.78 mmol, 1.00 equiv) in MeOH/THF/H₂O (5.00 mL/15.00 mL/18.30 mL) was added di-tert-butyl dicarbonate (0.78 g, 3.55 mmol, 2.00 equiv) and DMAP (0.02 g, 0.18 mmol, 0.10 equiv). The reaction was stirred at room temperature for 3.0 h. The mixture was added with H₂O (30 mL). The mixture was filtered through a Celite pad, and the solid was washed with ethyl acetate (3×30 mL) to afford 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.20 g, 76.05% yield) as a yellow solid. LC/MS: mass calcd. For C₃₉H₄₉N₁₅O₁₀: 887.38, found: 888.45 [M+H]⁺.

### Step 6: Synthesis of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate (INT81-023-9)

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate (INT61-04-OH-12), but the reaction time was 2.0 h. 1.20 g of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid was used, 1.10 g of desired product was obtained as a yellow solid (71.01% yield). LC/MS: mass calcd. For C₅₂H₆₃N₁₉O₁₂: 1145.49, found: 1146.50 [M+H]⁺.

### Step 7: Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid (PA-023)

The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid (INT60-022-0). 1.00 g of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was used, 400.00 mg of desired product was obtained as a white solid (39.16% yield). LC/MS: mass calcd. For C₅₁H₆₁N₁₉O₁₂: 1131.47, found: 1132.65 [M+H]⁺

Synthesis of Representative Compounds

### Example 28. Synthesis of Compound 1

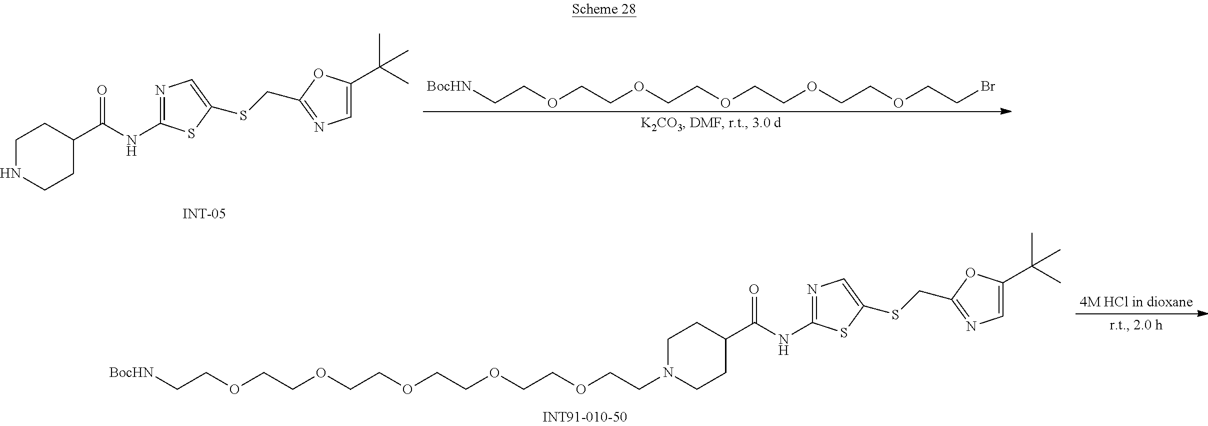 [SMILES: CC(C)(C)c1cnc(CSc2cnc(NC(=O)C3CCNCC3)s2)o1 ; CC(C)(C)OC(=O)NCCOCCOCCOCCOCCOCCBr ; CC(C)(C)OC(=O)NCCOCCOCCOCCOCCOCCN1CCC(C(=O)Nc2ncc(SCc3ncc(C(C)(C)C)o3)s2)CC1]

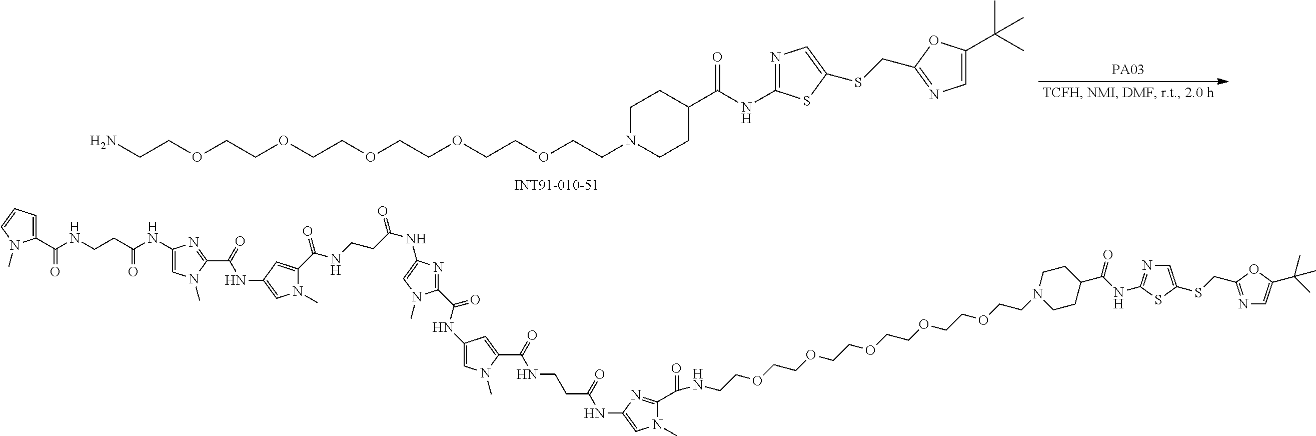 [SMILES: CC(C)(C)c1cnc(CSc2cnc(NC(=O)C3CCN(CCOCCOCCOCCOCCOCCN)CC3)s2)o1 ; Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)NCCOCCOCCOCCOCCOCCN2CCC(C(=O)Nc3ncc(SCc4ncc(C(C)(C)C)o4)s3)CC2)n1]

### Step 1: Synthesis of tert-butyl N-(17-[4-[(5-[[(5-tert-butyl-1,3-oxazol-2-yl) methyl]sulfanyl]-1,3-thiazol-2-yl)carbamoyl]piperidin-1-yl]-3,6,9,12,15-pentaoxaheptadecan-1-yl)carbamate (INT91-010-50)

To a solution of tert-butyl N-(17-bromo-3,6,9,12,15-pentaoxaheptadecan-1-yl) carbamate (100.00 mg, 0.22 mmol, 1.00 equiv) in DMF (1.50 mL) was added N-(5-[[(5-tert-butyl-1,3-oxazol-2-yl)methyl]sulfanyl]-1,3-thiazol-2-yl)piperidine-4-carboxamide, (89.92 mg, 0.24 mmol, 1.05 equiv) and K₂CO₃ (93.31 mmol, 0.68 mmol, 3.00 equiv). Then the reaction was stirred at r.t. for 3.0 d. The reaction mixture was filtered and the filtration was purified with reverse phase column under the condition: column, C18 column; mobile phase, MeCN in water, 5% to 50% gradient in 40 min; detector, UV 254 nm. The fractions were combined and concentrated under the vacuum. 130 mg desired product was obtained as colorless oil (77.00% yield). LC/MS: mass calcd. For C₃₄H₅₇N₅O₉S₂: 743.36, found: 744.55 [M+H]⁺.

### Step 2: Synthesis of 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51)

A solution of tert-butyl N-(17-[4-[(5-[[(5-tert-butyl-1,3-oxazol-2-yl) methyl]sulfanyl]-1,3-thiazol-2-yl)carbamoyl]piperidin-1-yl]-3,6,9,12,15-pentaoxaheptadecan-1-yl)carbamate (30.00 mg) in 4M HCl in 1,4-dioxane (1.00 mL) was stirred at rt for 2.0 h. The mixture was concentrated to afford 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (30.00 mg, crude) as an oil. LC/MS: mass calcd. For C₂₉H₄₉N₅O₇S₂: 643.31, found: 644.45 [M+H]⁺.

### Step 3: Synthesis of N-(5-[[(5-tert-butyl-1,3-oxazol-2-yl)methyl]sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001)

To a solution of 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (21.00 mg, 0.033 mmol, 1.05 equiv) in DMF (2.00 mL) was added NMI (21.00 mg, 0.26 mmol, 8.10 equiv), TCFH (10 mg, 0.07 mmol, 2.19 equiv) and 1-methyl-4-(3-(1-methyl-4-(1-methyl-4-(3-(1-methyl-4-(1-methyl-4-(3-(1-methyl-1H-pyrrole-2-carboxamido)propanamido)-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylic acid (30.00 mg, 0.032 mmol, 1.00 equiv). Then the reaction was stirred at r.t. for 2.0 h. The crude mixture in DMF (2.0 mL) was purified by Prep-HPLC with the following conditions (Column: XSelect CSH Prep C18 OBD Column, 5 um, 19*150 mm; Mobile Phase A: Water (10 mmol/L NH₄HCO₃), Mobile Phase B: ACN; Flow rate:25 mL/min; Gradient:20 B to 40 B in 20 min; 220 n; RT1:19.35; RT2; Injection Volume: 2 ml; Number Of Runs: 5) to afford N-(5-[[(5-tert-butyl-1,3-oxazol-2-yl)methyl]sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (21.20 mg, 41.00% yield) as white solid. HRMS: mass calcd. For C₇₁H₉₆N₂₂O₁₆S₂: 1576.6816, found: 1577.6870 [M+H]⁺.

### Example 29. Synthesis of Compound 004

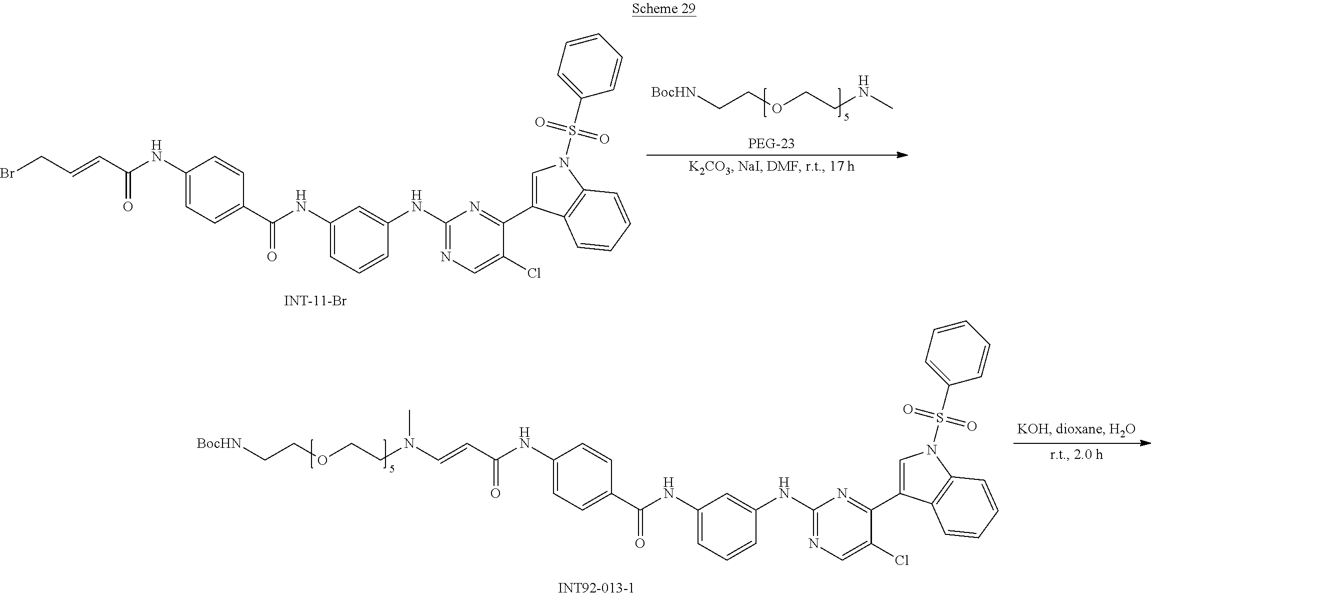

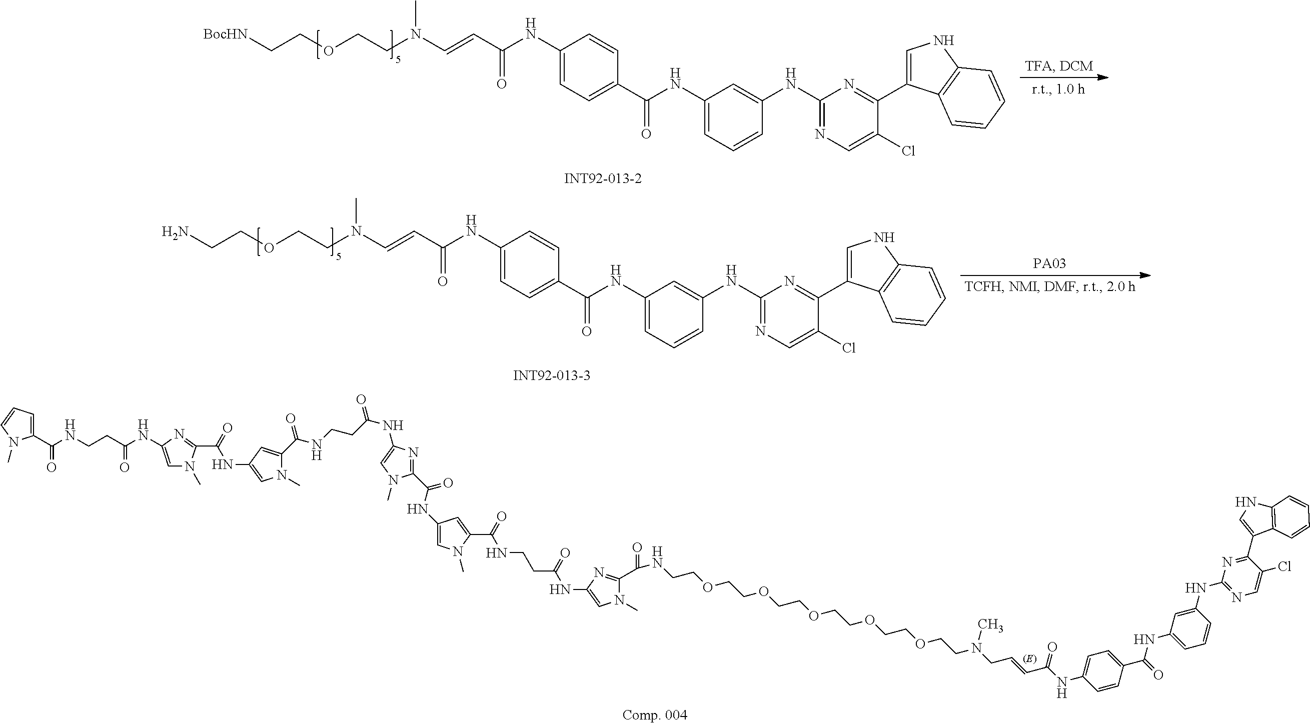

### Step 1: Synthesis of tert-butyl N-[(20E)-21-[(4-[[3-([4-[1-(benzenesulfonyl) indol-3-yl]-5-chloropyrimidin-2-yl]amino)phenyl]carbamoyl]phenyl)carbamoyl]-18-methyl-3,6,9,12,15-pentaoxa-18-azahenicos-20-en-1-yl]carbamate (INT92-013-1)

To a stirred solution of N-[3-([4-[1-(benzenesulfonyl)indol-3-yl]-5-chloropyrimidin-2-yl]amino)phenyl]-4-[(2E)-4-bromobut-2-enamido]benzamide (150.00 mg, 0.20 mmol, 1.00 equiv) in DMF (4.00 mL) was added tert-butyl N-(5,8,11,14,17-pentaoxa-2-azanonadecan-19-yl)carbamate (79.75 mg, 0.20 mmol, 1.00 equiv), K₂CO₃ (83.81 mg, 0.60 mmol, 3.00 equiv) and NaI (60.60 mg, 0.40 mmol, 2.00 equiv) in portions at room temperature. The resulting mixture was stirred for 17.0 h at room temperature. The solid was filtered out and the filtrate was purified by reverse flash chromatography with the following conditions: column, C18 silica gel; mobile phase, ACN in water (0.05% TFA), 35% to 55% gradient in 20 min; detector, UV 254 nm. The fractions were combined and concentrated. Tert-butyl N-[(20E)-21-[(4-[[3-([4-[1-(benzenesulfonyl)indol-3-yl]-5-chloropyrimidin-2-yl]amino)phenyl]carbamoyl]phenyl)carbamoyl]-18-methyl-3,6,9,12,15-pentaoxa-18-azahenicos-20-en-1-yl]carbamate (200.00 mg, 88.40% yield) was obtained as a yellow oil. LC/MS: mass calcd. For C₅₃H₆₃ClN₈O₁₁S: 1054.40, found: 1055.65 [M+H]⁺.

### Step 2: Synthesis of tert-butyl N-[(20E)-21-([4-[(3-[[5-chloro-4-(JH-indol-3-yl)pyrimidin-2-yl[amino]phenyl)carbamoyl]phenyl]carbamoyl)-18-methyl-3,6,9,12,15-pentaoxa-18-azahenicos-20-en-1-yl]carbamate (INT92-013-2)

To a stirred solution of tert-butyl N-[(20E)-21-[(4-[[3-([4-[1-(benzenesulfonyl) indol-3-yl]-5-chloropyrimidin-2-yl]amino)phenyl]carbamoyl]phenyl)carbamoyl]-18-methyl-3,6,9,12,15-pentaoxa-18-azahenicos-20-en-1-yl]carbamate (190.00 mg, 0.18 mmol, 1.00 equiv) in dioxane (3.00 mL) was added 1M KOH in H₂O (3.0 mL) dropwise at room temperature. The resulting mixture was stirred for 2.0 h at room temperature. The resulting mixture was concentrated under vacuum. The residue was purified by reverse flash chromatography with the following conditions: column, C18 silica gel; mobile phase, ACN in water (0.05% TFA), 30% to 50% gradient in 20 min; detector, UV 254 nm. The fractions were combined and concentrated. Tert-butyl N-[(20E)-21-([4-[(3-[[5-chloro-4-(1H-indol-3-yl)pyrimidin-2-yl]amino]phenyl)carbamoyl]phenyl]carbamoyl)-18-methyl-3,6,9,12,15-pentaoxa-18-azahenicos-20-en-1-yl]carbamate (100.00 mg, 43.48% yield) was obtained as a yellow oil. LC/MS: mass calcd. For C₄₇H₅₉ClN₈O₉: 914.41, found: 915.65 [M+H]⁺.

### Step 3: Synthesis of (20E)-1-amino-N-[4-[(3-[[5-chloro-4-(1H-indol-3-yl) pyrimidin-2-yl[amino]phenyl)carbamoyl]phenyl]-18-methyl-3,6,9,12,15-pentaoxa-18-azadocos-20-en-22-amide (INT92-013-3)

The procedure was the same as (INT90-050-6). 100.00 mg of tert-butyl N-[(20E)-21-([4-[(3-[[5-chloro-4-(1H-indol-3-yl)pyrimidin-2-yl]amino]phenyl)carbamoyl]phenyl]carbamoyl)-18-methyl-3,6,9,12,15-pentaoxa-18-azahenicos-20-en-1-yl]carbamate was used, 100.00 mg crude of desired product was obtained as yellow oil. LC/MS: mass calcd. For C₄₂H₅₁ClN₈O₇: 814.36, found: 815.55 [M+H]⁺.

### Step 4: Synthesis of N-[5-([2-[(2-[[5-([2-[(2-[[(20E)-21-([4-[(3-[[5-chloro-4-(1H-indol-3-yl)pyrimidin-2-yl]amino]phenyl)carbamoyl]phenyl]carbamoyl)-18-methyl-3,6,9,12,15-pentaoxa-18-azahenicos-20-en-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-1-methyl-4-[3-[(1-methylpyrrol-2-yl)formamido]propanamido]imidazole-2-carboxamide (Comp. 004)

The procedure was the same as N-(5-[[(5-tert-butyl-1,3-oxazol-2-yl) methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001). 45.00 mg of (20E)-1-amino-N-[4-[(3-[[5-chloro-4-(1H-indol-3-yl)pyrimidin-2-yl]amino]phenyl)carbamoyl]phenyl]-18-methyl-3,6,9,12,15-pentaoxa-18-azadocos-20-en-22-amide was used, 8.60 mg of desired product was obtained as white solid (8.54% yield). HRMS: mass calcd. For C₈₄H₉₈ClN₂₅O₁₆: 1747.7312, found: 1748.7334 [M+H]⁺.

### Example 30. Synthesis of Synthesis of Compound 010

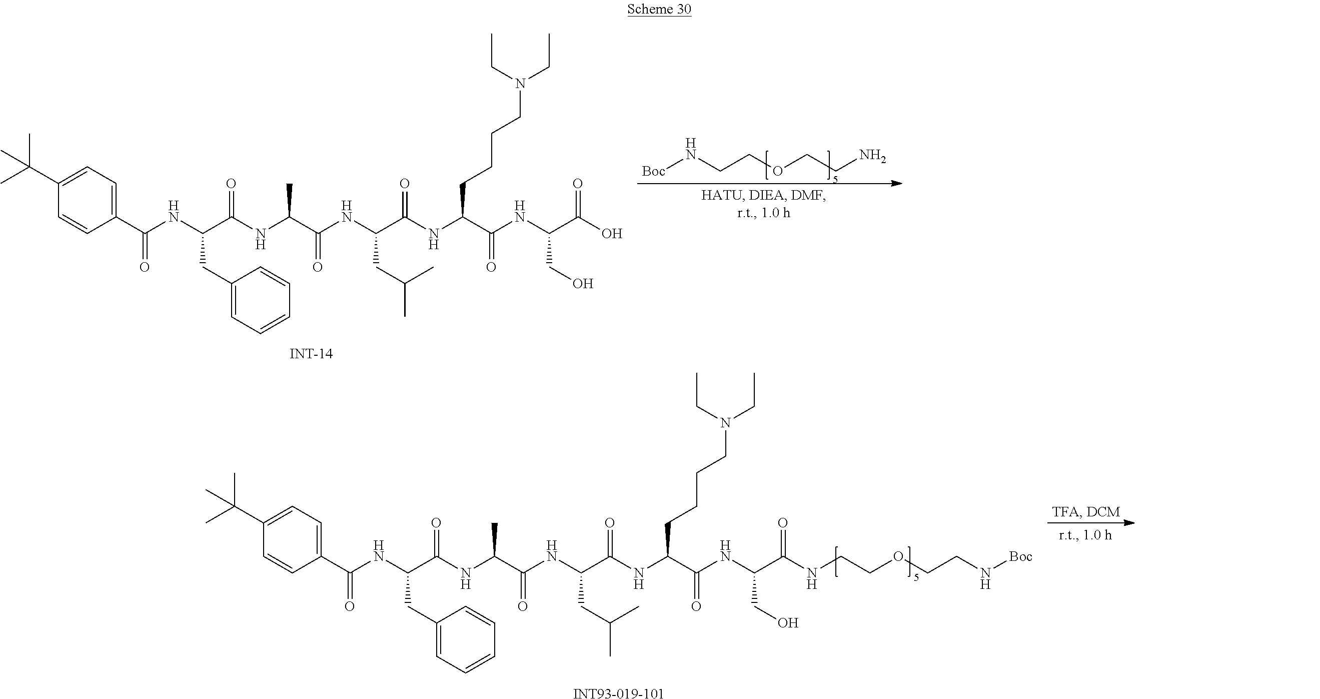

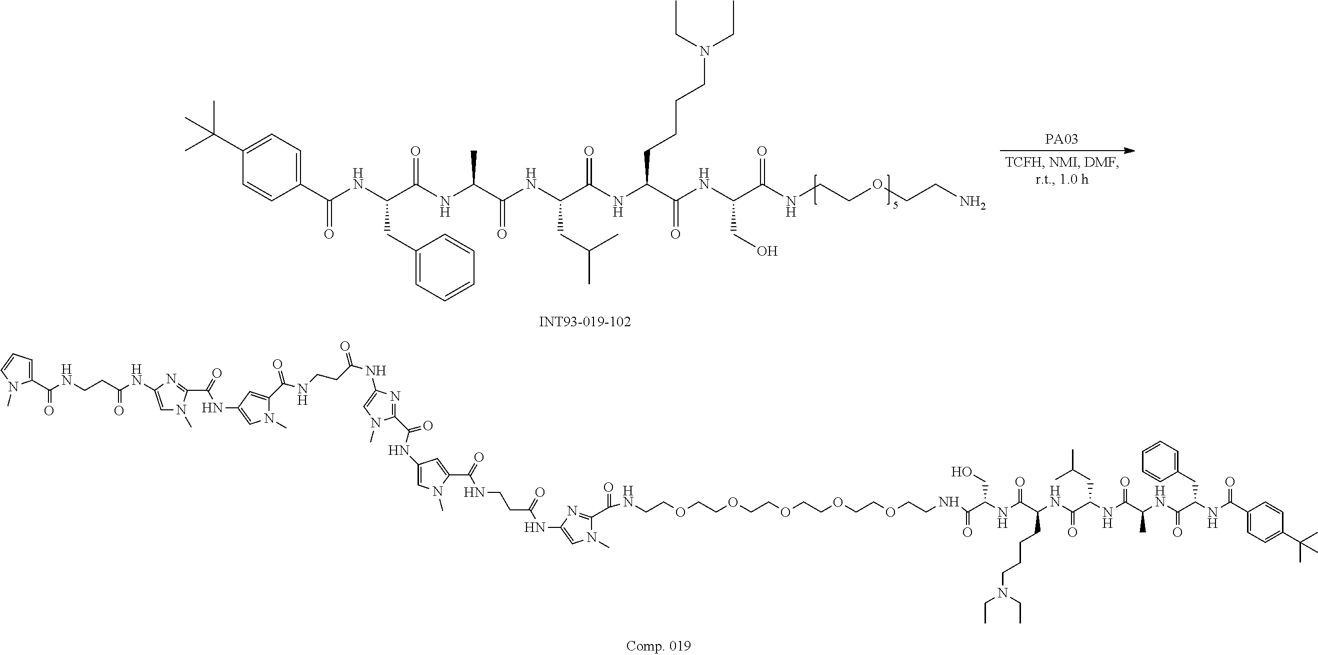

### Step 1: Synthesis of tert-butyl N-[17-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl]carbamate (INT93-019-101)

The procedure was the same as (INT60-017-10), but the reaction time was 1.0 h. 100.00 mg of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoic acid was used, 50.00 mg of desired product was obtained as yellow solid (34.15% yield). LC/MS: mass calcd. For C₅₉H₉₈N₈O₁₄: 1142.72, found: 522.70[1/2(M-Boc)+H]⁺.

### Step 2: Synthesis of (2S)-N-[(1S)-1-[(17-amino-3,6,9,12,15-pentaoxaheptadecan-1-yl)carbamoyl]-2-hydroxyethyl]-2-[(2S)-2-[(2S)-2-[(2S)-2-[(4-tert-butylphenyl)formamido]-3-phenylpropanamido]propanamido]-4-methylpentanamido]-6-(diethylamino)hexanamide (INT93-019-102)

The procedure was the same as Synthesis of (2-{2-[(4-tert-butylphenyl)formamido]ethoxy}ethoxy)acetic acid (INT-29-110), 50.00 mg of tert-butyl N-[17-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl]carbamate was used, 50.00 mg crude of desired product was obtained as yellow solid. LC/MS: mass calcd. For C₅₄H₉₀N₈O₁₂: 1042.66, found: 1043.90[M+H]⁺.

### Step 3: Synthesis of N-[5-[(2-[[2-([5-[(2-[[2-([17-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl]carbamoyl)-1-methylimidazol-4-yl]carbamoyl]ethyl)carbamoyl]-1-methylpyrrol-3-yl]carbamoyl)-1-methylimidazol-4-yl]carbamoyl]ethyl)carbamoyl]-1-methylpyrrol-3-yl]-1-methyl-4-[3-[(1-methylpyrrol-2-yl)formamido]propanamido]imidazole-2-carboxamide (Comp 010)

The procedure was the same as N-(5-[[(5-tert-butyl-1,3-oxazol-2-yl) methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001), but the reaction time was 1.0 h. 28.00 mg of 1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-carboxylic acid was used, 10.70 mg of desired product was obtained as white solid (18.10% yield). HRMS: mass calcd. For C₉₆H₁₃₇N₂₅O₂₁: 1976.0421, found: 1977.0458[M+H]⁺.

### Example 31. Synthesis of Compound 096

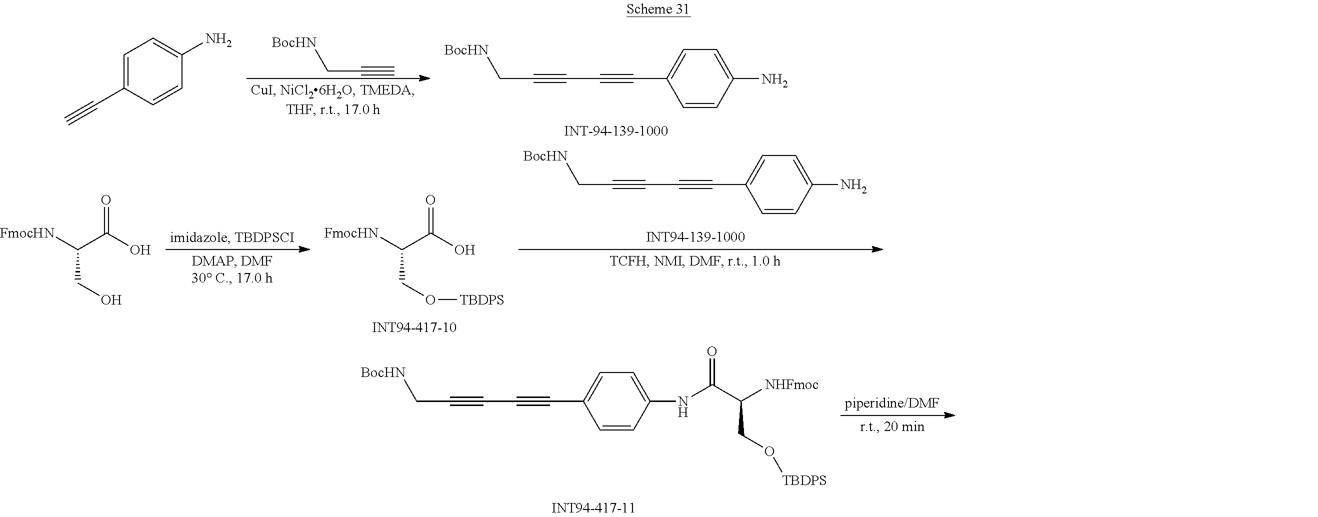

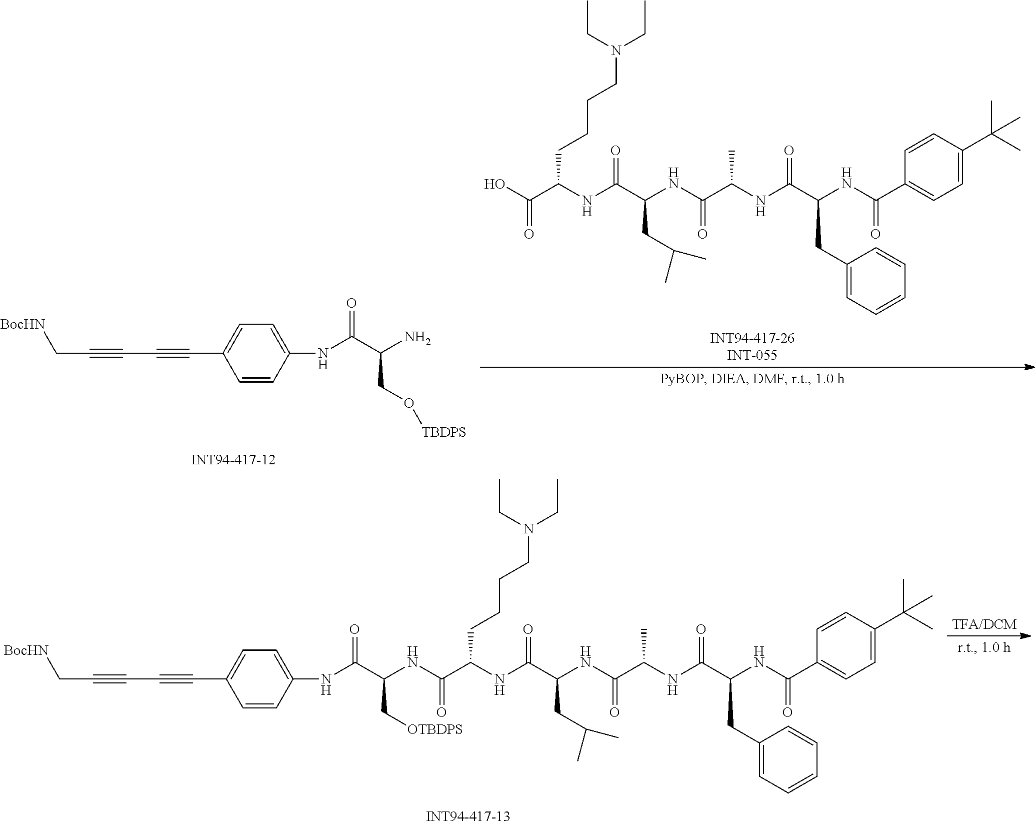

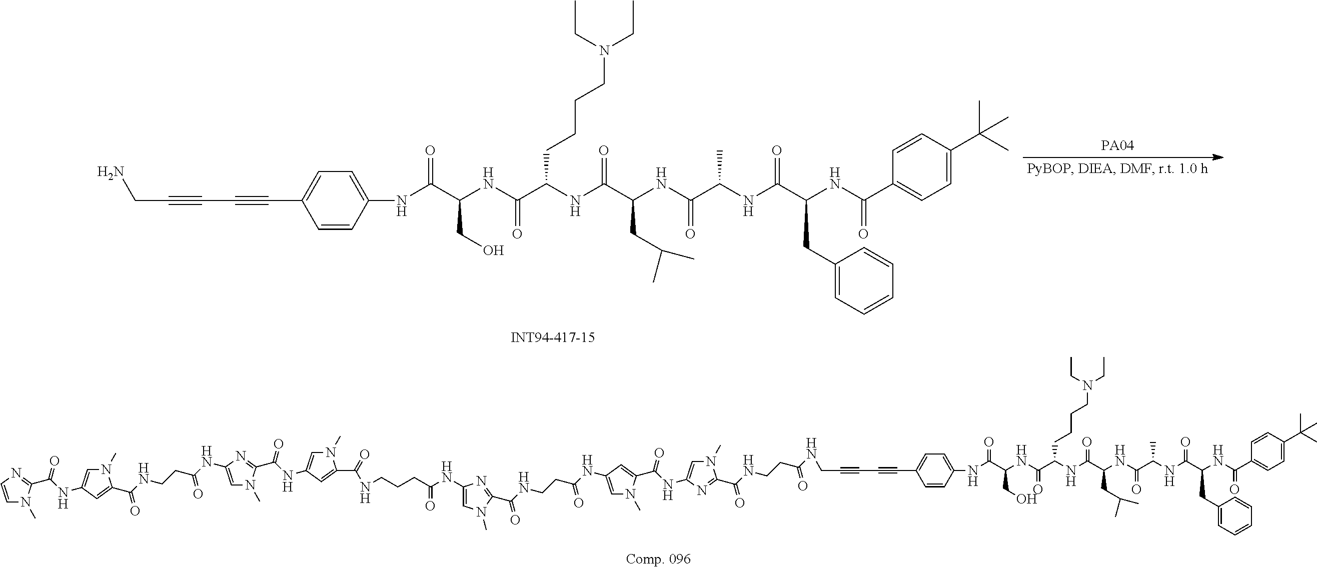 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)Nc1ccc(C#CC#CCN)cc1 ; CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)Nc1ccc(C#CC#CCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cc1]

### Step 1: Synthesis of tert-butyl N-[5-(4-aminophenyl)penta-2,4-diyn-1-yl] carbamate (INT-94-139-1000)

To a stirred solution of CuI (56.90 mg, 0.30 mmol, 0.05 equiv) and NiCl₂·6H₂O (71.01 mg, 0.30 mmol, 0.05 equiv) in THF (20.00 mL) was added TMEDA (138.87 mg, 1.20 mmol, 0.20 equiv) dropwise at room temperature under air atmosphere. The resulting mixture was stirred for 5 min at room temperature under air atmosphere. To the above mixture was added 4-ethynylaniline (700.00 mg, 5.98 mmol, 1.00 equiv) and tert-butyl N-(prop-2-yn-1-yl)carbamate (463.67 mg, 2.99 mmol, 0.50 equiv) in THF (10.00 mL) in portions at room temperature. The resulting mixture was stirred for additional 17.0 h at room temperature. The reaction was quenched by addition of H₂O (50 mL) at room temperature. The resulting mixture was extracted with EA (3×80 mL). The combined organic layers were washed with brine (1×50 mL), fried over anhydrous Ns₂SO₄. After filtration, the filtrate was concentrated under reduced pressure. The residue was purified by silica gel column chromatography, eluted with PE/EA (3:1) to afford tert-butyl N-[5-(4-aminophenyl)penta-2,4-diyn-1-yl]carbamate (600.00 mg, 37.15% yield) as a yellow solid. LC/MS: mass calcd. For C₁₆H₁₈N₂O₂: 270.14, found: 215.05 [M−tBu+1+H]⁺.

### Step 2: Synthesis of (2R)-3-[(tert-butyldiphenylsilyl)oxy]-2-{[(9H-fluoren-9-ylmethoxy)carbonyl] amino}propanoic acid (INT94-417-10)

To a stirred solution of (2R)-2-{[(9H-fluoren-9-ylmethoxy)carbonyl]amino}-3-hydroxypropanoic acid (2.00 g, 6.11 mmol, 1.00 equiv) in DMF (25.00 mL) was added DMAP (0.07 g, 0.61 mmol, 0.10 equiv), Imidazole (0.83 g, 12.22 mmol, 2.00 equiv) and TBDPSCl (3.36 g, 12.22 mmol, 2.00 equiv) in portions at room temperature. The resulting mixture was stirred for 17.0 h at 30 degrees C.

The resulting mixture was concentrated under vacuum. The residue was purified by silica gel column chromatography, eluted with CH₂Cl₂/MeOH (10:1) to afford (2R)-3-[(tert-butyldiphenylsilyl)oxy]-2-{[(9H-fluoren-9-ylmethoxy)carbonyl]amino} propanoic acid (1.70 g, 49.24% yield) as a white solid. LC/MS: mass calcd. For C₃₄H₃₅NO₅Si: 565.23, found: 566.45 [M+H]⁺.

### Step 3: Synthesis of 9H-fluoren-9-ylmethyl N-[(1S)-1-[(4-{5-[(tert-butoxycarbonyl)amino]penta-1,3-diyn-1-yl}phenyl)carbamoyl]-2-[(tert-butyldiphenylsilyl)oxy]ethyl]carbamate (INT94-417-11)

The procedure was the same as N-(5-[[(5-tert-butyl-1,3-oxazol-2-yl) methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001), but the reaction time was 1.0 h. 520.00 mg of (2S)-3-[(tert-butyldiphenylsilyl)oxy]-2-{[(9H-fluoren-9-ylmethoxy)carbonyl] amino}propanoic acid was used. After the reaction, the reaction mixture was poured into ice-water, the obtained solid was purified by silica gel chromatography. 350.00 mg of desired product was obtained as yellow solid (46.55% yield). LC/MS: mass calcd. For C₅₀H₅₁N₃O₆Si: 817.35, found: 818.70[M+H]⁺.

### Step 4: Synthesis of Tert-butyl N-(5-{4-[(2S)-2-amino-3-[(tert-butyldiphenylsilyl) oxy]propanamido]phenyl}penta-2,4-diyn-1-yl)carbamate (INT94-417-12)

To a stirred solution of 9H-fluoren-9-ylmethyl N-[(1S)-1-[(4-{5-[(tert-butoxycarbonyl)amino]penta-1,3-diyn-1-yl}phenyl)carbamoyl]-2-[(tert-butyldiphenylsilyl)oxy]ethyl]carbamate (330.00 mg, 0.40 mmol, 1.00 equiv) in DMF (5.00 mL) was added piperidine (0.50 mL) at room temperature. The resulting mixture was stirred for 20 min at room temperature. The reaction mixture was purified by reverse flash chromatography with the following conditions: column, C18 silica gel; mobile phase, ACN in water (0.05% TFA), 35% to 50% gradient in 20 min; detector, UV 254 nm. The fractions were combined and concentrated. Tert-butyl N-(5-{4-[(2S)-2-amino-3-[(tert-butyldiphenylsilyl)oxy]propanamido]phenyl}penta-2,4-diyn-1-yl)carbamate (160.00 mg, 66.57% yield) was obtained as yellow oil. LC/MS: mass calcd. For C₃₅H₄₁N₃O₄Si: 595.29, found: 596.25[M+H]⁺.

### Step 5: Synthesis of tert-butyl N-(5-{4-[(2S)-3-[(tert-butyldiphenylsilyl)oxy]-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]propanamido]phenyl}penta-2,4-diyn-1-yl)carbamate (INT94-417-13)

The procedure was the same as (INT61-04-OH-21). 45.00 mg of (2S)-2-[(2S)-2-[(2S)-2-[(2S)-2-[(4-tert-butylphenyl)formamido]-3-phenylpropanamido]propanamido]-4-methylpentanamido]-6-(diethylamino)hexanoic acid was used, 50.00 mg of desired product was obtained as yellow solid (60.63% yield). LC/MS: mass calcd. For C₇₄H₉₈N₈O₉Si: 1270.72, found: 1272.00[M+H]⁺.

### Step 6: Synthesis of (2S)-N-[(1S)-1-{[4-(5-aminopenta-1,3-diyn-1-yl) phenyl]carbamoyl}-2-hydroxyethyl]-2-[(2S)-2-[(2S)-2-[(2S)-2-[(4-tert-butylphenyl)formamido]-3-phenylpropanamido]propanamido]-4-methylpentanamido]-6-(diethylamino)hexanamide (INT94-417-15)

The procedure was the same as (INT-29-110). 45.00 mg of tert-butyl N-(5-{4-[(2S)-3-[(tert-butyldiphenylsilyl) oxy]-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]propanamido]phenyl}penta-2,4-diyn-1-yl)carbamate was used, 45.00 mg crude of desired product was obtained as yellow solid. LC/MS: mass calcd. For C₅₃H₇₂N₈O₇: 932.55, found: 933.95[M+H]⁺.

### Step 7: Synthesis of N-{5-[(3-{[2-({2-[(5-{[2-({2-[(5-{4-[(2S)-2-[(2S)-2-[(2S)-2-[(2S)- 2-[(2S)-2-[(4-tert-butylphenyl)formamido]-3-phenylpropanamido]propanamido]-4-methylpentanamido]-6-(diethylamino)hexanamido]-3-hydroxypropanamido]phenyl}penta-2,4-diyn-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 096)

The procedure was the same as (INT61-04-OH-21), but the reaction mixture was purified by Prep-HPLC. 40.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 3.50 mg of desired product was obtained as white solid (4.53% yield). HRMS: mass calcd. For C₁₀₄H₁₃₁N₂₉O₁₈: 2074.0227, found: 2075.0222[M+H]⁺.

### Example 32. Synthesis of Compound 098

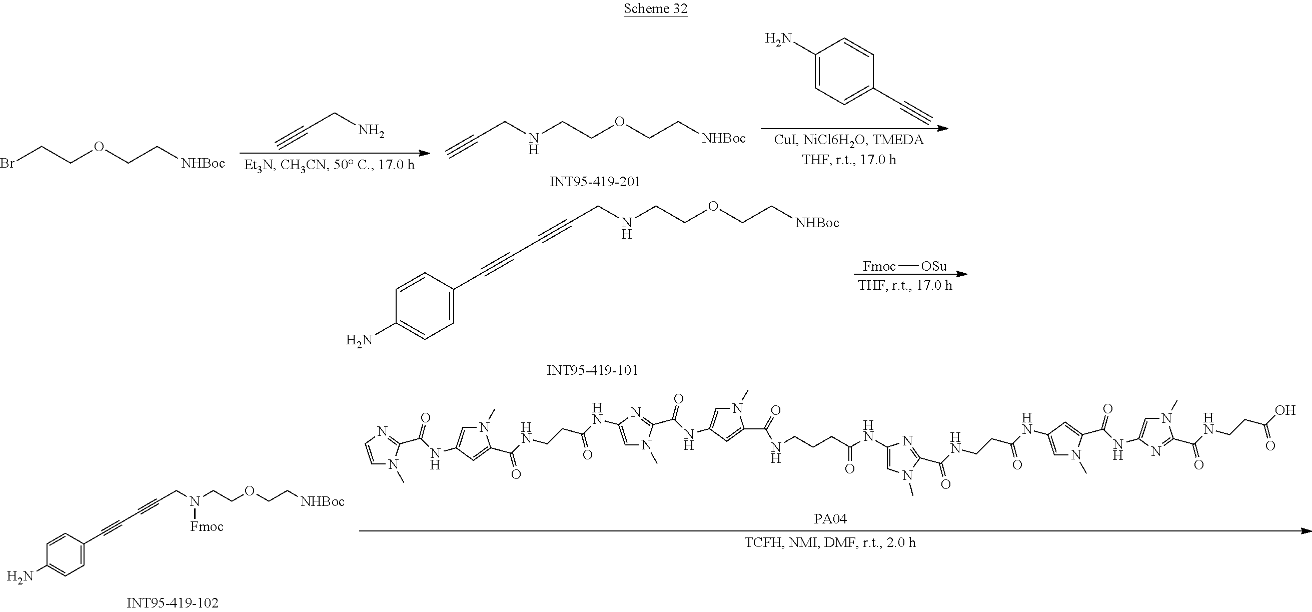

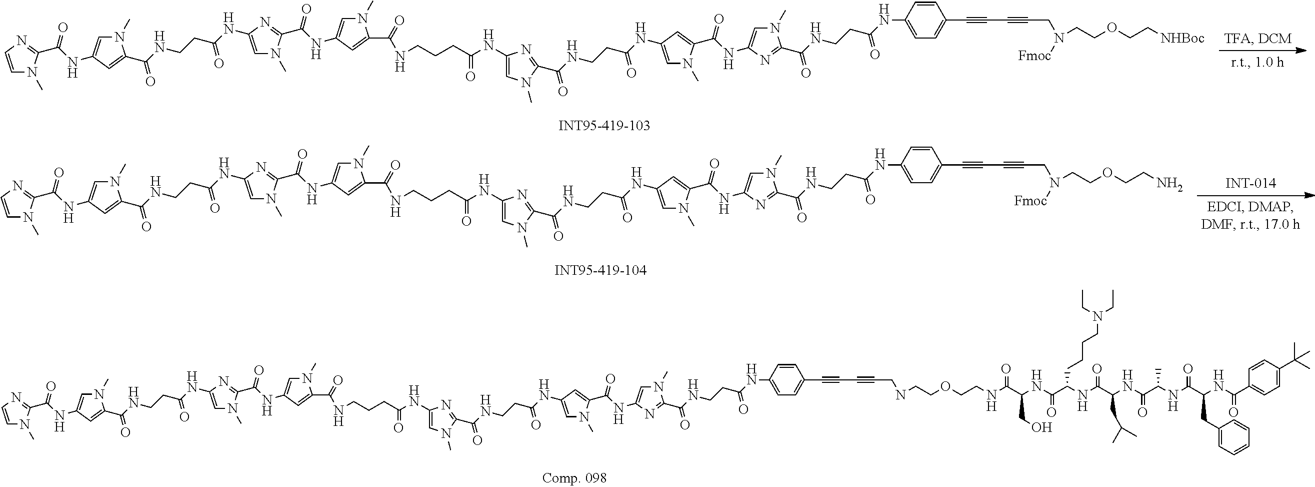

### Step 1: Synthesis of tert-butyl N-{2-[2-(prop-2-yn-1-ylamino)ethoxy]ethyl} carbamate (INT95-419-201)

To a solution of tert-butyl N-[2-(2-bromoethoxy)ethyl]carbamate (1.00 g, 3.73 mmol, 1.00 equiv) in ACN (10.00 mL) was added Et₃N (1.13 g, 11.187 mmol, 3.00 equiv) and 2-propynylamine (2.05 g, 37.29 mmol, 10.00 equiv). Then the reaction was stirred for 17.0 h at 50 degrees C. The reaction mixture was purified by reverse flash chromatography with the following conditions: column, C18 silica gel; mobile phase, ACN in water (0.05% NH₄HCO₃), 5% to 40% gradient in 60 min; detector, UV 220 n. The fractions were combined and concentrated to afford tert-butyl N-{2-[2-(prop-2-yn-1-ylamino)ethoxy]ethyl}carbamate (600.00 mg, 66.40% yield) as a yellow oil. LC/MS: mass calcd. For C₁₂H₂₂N₂O₃: 242.16, found: 243.20 [M+H]⁺.

### Step 2: Synthesis of tert-butyl N-[2-(2-{[5-(4-aminophenyl)penta-2,4-diyn-1-yl]amino}ethoxy)ethyl]carbamate (INT95-419-101)

The procedure was the same as tert-butyl N-[5-(4-aminophenyl)penta-2,4-diyn-1-yl]carbamate (INT94-139-1000), but the product was purified by reverse phase column. 300.00 mg of tert-butyl N-{2-[2-(prop-2-yn-1-ylamino)ethoxy]ethyl} carbamate was used, 250.00 mg of desired product was obtained as yellow oil (56.49% yield). LC/MS: mass calcd. For C₂₀H₂₇N₃O₃: 357.21, found: 358.20 [M+H]⁺.

### Step 3: Synthesis of tert-butyl N-[2-(2-{[5-(4-aminophenyl)penta-2,4-diyn-1-yl] [(9H-fluoren-9-ylmethoxy)carbonyl]amino}ethoxy)ethyl]carbamate (INT95-419-102)

To a stirred solution of tert-butyl N-[2-(2-{[5-(4-aminophenyl)penta-2,4-diyn-1-yl]amino}ethoxy)ethyl]carbamate amine (100.00 mg, 0.27 mmol, 1.00 equiv) in THF (3.00 mL) was added 2,5-dioxopyrrolidin-1-yl 9H-fluoren-9-ylmethyl carbonate (136.00 mg, 0.40 mmol, 1.50 equiv) in portions at room temperature. The resulting mixture was stirred for 17.0 h at room temperature. The resulting mixture was concentrated under vacuum. The residue was purified by TLC-plate, eluted with DCM/MeOH (10:1) to afford tert-butyl N-[2-(2-{[5-(4-aminophenyl)penta-2,4-diyn-1-yl][(9H-fluoren-9-ylmethoxy)carbonyl]amino}ethoxy)ethyl]carbamate (100.00 mg, 64.60% yield) as a yellow oil. LC/MS: mass calcd. For C₃₅H₃₇N₃O₅: 579.27, found: 602.50[M+Na]⁺.

### Step 4: Synthesis of tert-butyl N-[2-(2-{[(9H-fluoren-9-ylmethoxy)carbonyl][5-(4-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}phenyl)penta-2,4-diyn-1-yl]amino}ethoxy)ethyl]carbamate (INT95-419-103)

The procedure was the same as N-(5-[[(5-tert-butyl-1,3-oxazol-2-yl) methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001), but the reaction mixture was purified by reverse phase column. 120.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 50.00 mg of desired product was obtained as yellow solid (28.07% yield). LC/MS: mass calcd. For C₈₆H₉₆N₂₄O₁₆: 1720.74, found: 861.90 [M/2+H]⁺.

### Step 5: Synthesis of 9H-fluoren-9-ylmethyl N-[2-(2-aminoethoxy)ethyl]-N-[5-(4-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}phenyl)penta-2,4-diyn-1-yl]carbamate (INT95-419-104)

The procedure was the same as (INT-29-110). 40.00 mg of tert-butyl N-[2-(2-{[(9H-fluoren-9-ylmethoxy) carbonyl][5-(4-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}phenyl)penta-2,4-diyn-1-yl]amino}ethoxy)ethyl]carbamate was used, 40.00 mg crude of desired product was obtained as yellow oil. LC/MS: mass calcd. For C₈₁H₈₈N₂₄O₁₄: 1620.69, found: 811.85 [M/2+H]⁺.

### Step 6: Synthesis of N-{5-[(3-{[2-({2-[(5-{[2-({2-[(4-{5-[(2-{2-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]ethoxy}ethyl)amino]penta-1,3-diyn-1-yl}phenyl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 098)

The procedure was the same as (INT61-025-20), but the reaction temperature was r.t. 15.00 mg of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoic acid was used, 4.10 mg of desired product was obtained as white solid (10.15% yield). HRMS: mass calcd. For C₁₀₈H₁₄₀N₃₀O₁₉: 2161.0911, found: 2162.1060 [M+H]⁺.

### Example 33. Synthesis of Compound 071

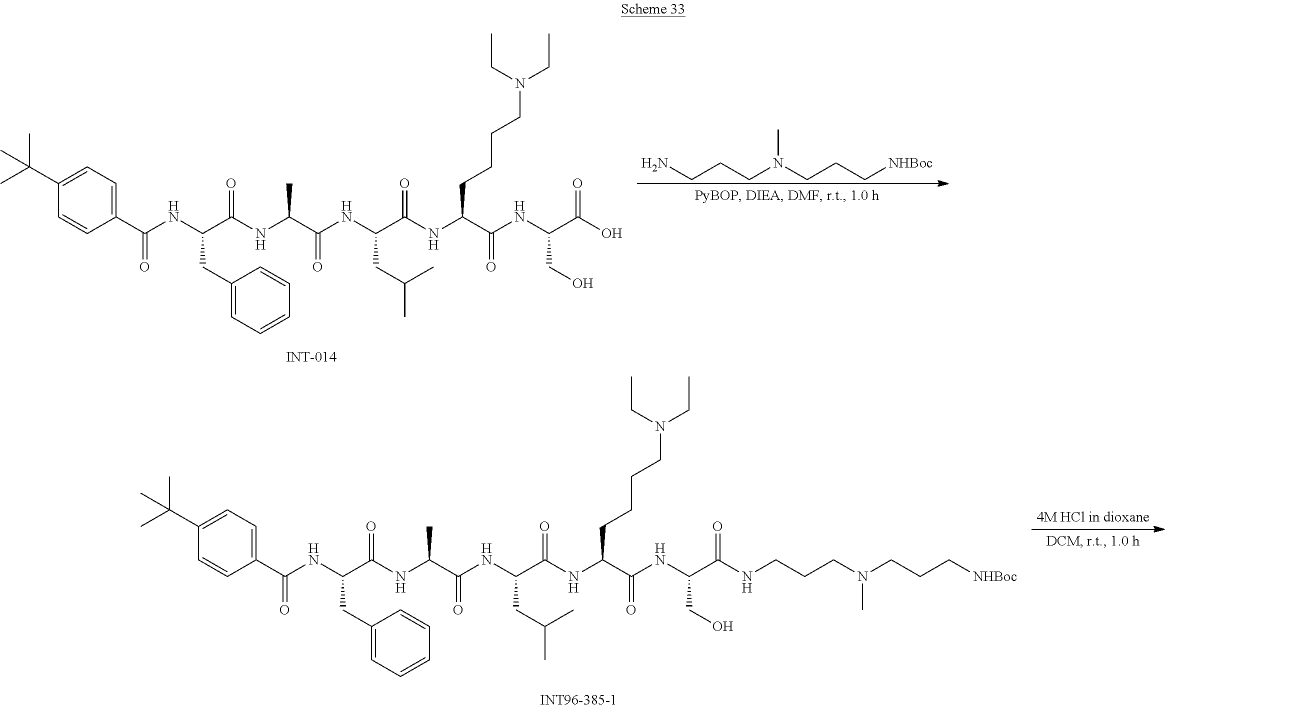 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)O ; CN(CCCN)CCCNC(=O)OC(C)(C)C ; CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCCN(C)CCCNC(=O)OC(C)(C)C]

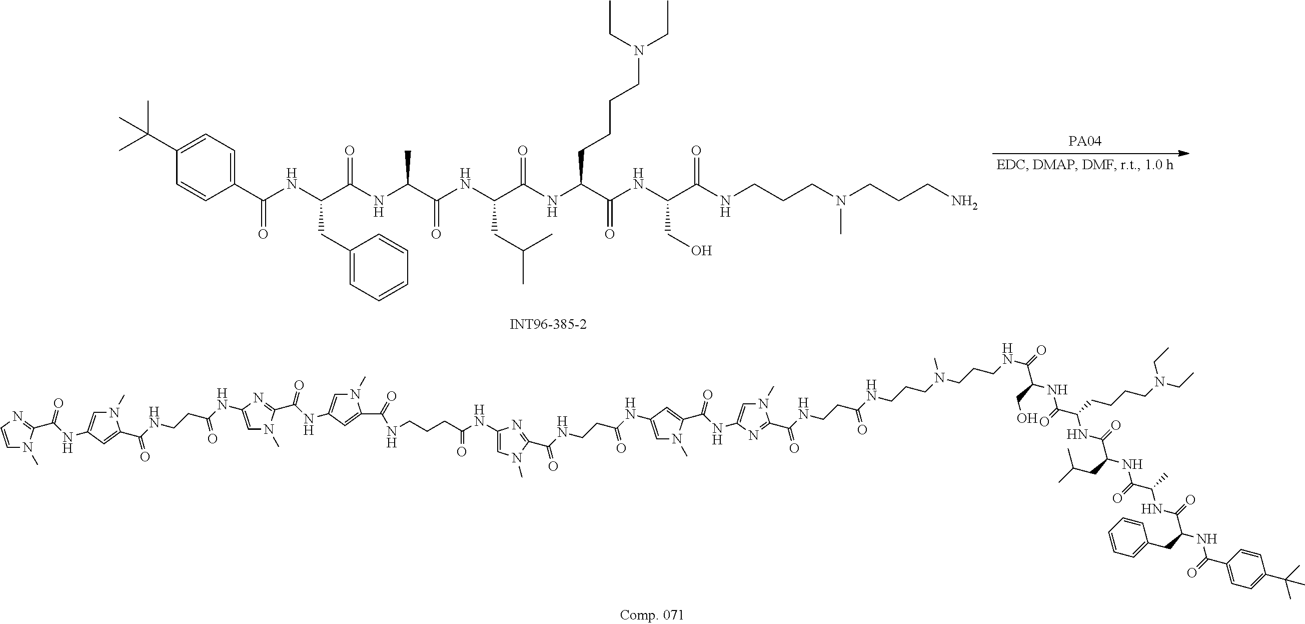 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCCN(C)CCCN ; CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCCN(C)CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C]

### Step 1: Synthesis of tert-butyl N-[3-({3-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]propyl} (methyl)amino)propyl]carbamate (INT96-385-1)

The procedure was the same as (INT61-04-OH-21). 40.00 mg of (2S)-2-[(2S)-2-[(2S)-2-[(2S)-2-[(2S)-2-[(4-tert-butylphenyl) formamido]-3-phenylpropanamido]propanamido]-4-methylpentanamido]-6-(diethylamino)hexanamido]-3-hydroxypropanoic acid was used, 50.00 mg of desired product was obtained as light yellow oil (96.82% yield). LC/MS: mass calcd. For C₅₄H₈₉N₉O₉: 1007.69, found: 505.30 [M/2+H]⁺.

### Step 2: Synthesis of (2S)-N-[(1S)-1-({3-[(3-aminopropyl)(methyl)amino]propyl}carbamoyl)-2-hydroxyethyl]-2-[(2S)-2-[(2S)-2-[(2S)-2-[(4-tert-butylphenyl)formamido]-3-phenylpropanamido]propanamido]-4-methylpentanamido]-6-(diethylamino)hexanamide (INT96-385-2)

The procedure was the same as (INT91-010-51). 50.00 mg of tert-butyl N-[3-({3-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]propyl}(methyl)amino)propyl]carbamate was used, 50.00 mg crude of desired product was obtained as colorless oil. LC/MS: mass calcd. For C₄₉H₈₁N₉O₇: 907.63, found: 908.90 [M+H]⁺.

### Step 3: Synthesis of N-(5-{[3-({2-[(2-{[5-({2-[(2-{[3-({3-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]propyl}(methyl)amino)propyl]carbamoyl}ethyl)carbamoyl]-1-methylimidazol-4-yl}carbamoyl)-1-methylpyrrol-3-yl]carbamoyl}ethyl)carbamoyl]-1-methylimidazol-4-yl}carbamoyl)propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Comp. 071)

The procedure was the same as (INT61-025-20), but the reaction temperature was r.t. and reaction time was 1.0 h. 54.00 mg of 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylic acid was used, 8.80 mg of desired product was obtained as white solid (8.29% yield). HRMS: mass calcd. For C₁₀₀H₁₄₀N₃₀O₁₈: 2049.0962, found: 2050.1128 [M+H]⁺.

### Example 34. Synthesis of Synthesis of Compound 109

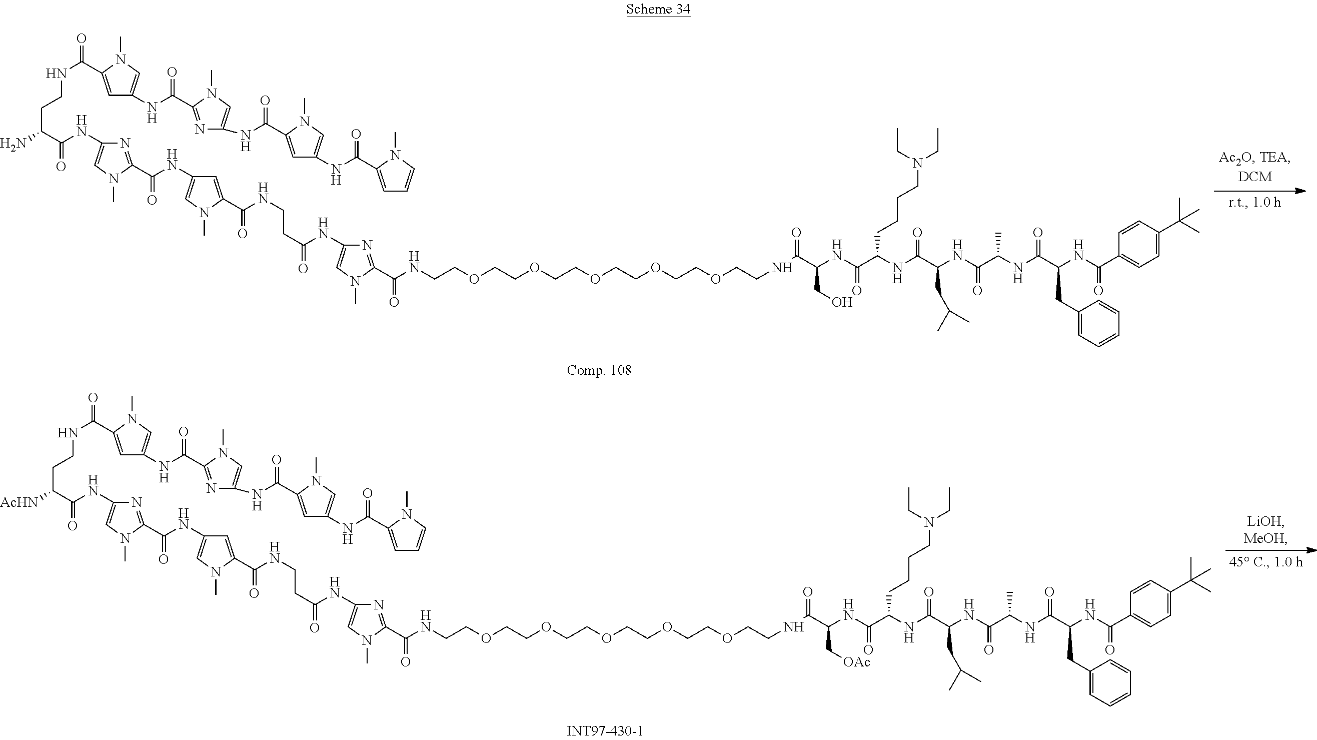

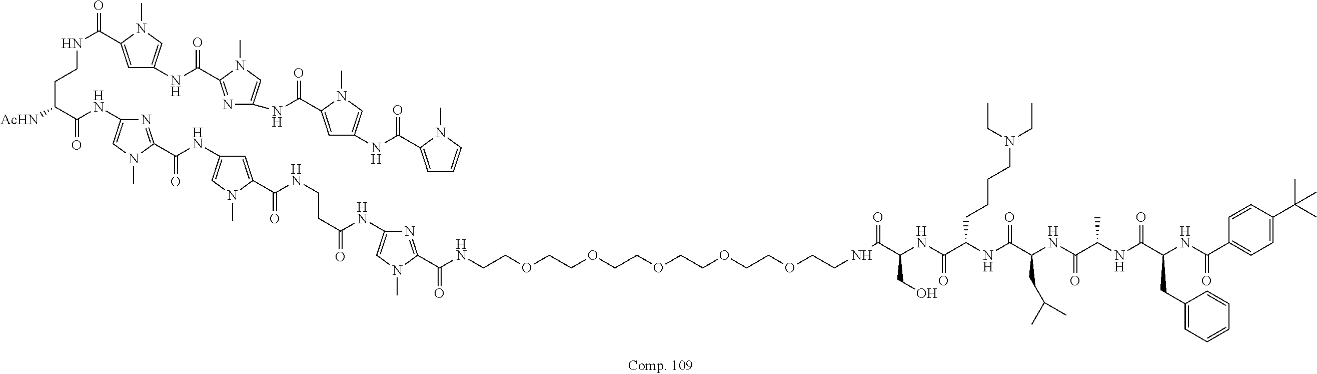 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCOCCOCCOCCNC(=O)c1nc(NC(=O)CCNC(=O)c2cc(NC(=O)c3nc(NC(=O)[C@@H](CCNC(=O)c4cc(NC(=O)c5nc(NC(=O)c6cc(NC(=O)c7cccn7C)cn6C)cn5C)cn4C)NC(C)=O)cn3C)cn2C)cn1C]

### Step 1: Synthesis of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-2-({17-[(4-{3-[(4-{4-[(2R)-2-acetamido-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-amido}-1-methylpyrrol-2-yl)formamido]propanamido}-1-methylimidazol-2-yl)formamido]-3,6,9,12,15-pentaoxaheptadecan-1-yl}carbamoyl)ethyl acetate (INT97-430-1)

To a solution of 4-[(2R)-2-amino-4-[(1-methyl-4-{1-methyl-4-[1-methyl-4-(1-methylpyrrole-2-amido)pyrrole-2-amido]imidazole-2-amido}pyrrol-2-yl)formamido]butanamido]-N-{5-[(2-{[2-({17-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl}carbamoyl)-1-methylimidazol-4-yl]carbamoyl}ethyl)carbamoyl]-1-methylpyrrol-3-yl}-1-methylimidazole-2-carboxamide (20.00 mg, 0.01 mmol, 1.00 equiv) in DCM (1.50 mL) was added Ac₂O (6.95 mg, 0.07 mmol, 7.00 equiv), TEA (6.89 mg, 0.07 mmol, 7.00 equiv). The resulting mixture was stirred at room temperature for 1.0 h. The resulting mixture was concentrated under vacuum. (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-2-({17-[(4-{3-[(4-{4-[(2R)-2-acetamido-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-amido}-1-methylpyrrol-2-yl)formamido]propanamido}-1-methylimidazol-2-yl)formamido]-3,6,9,12,15-pentaoxaheptadecan-1-yl}carbamoyl)ethyl acetate (20.00 mg crude) was obtained as yellow oil. LC/MS: mass calcd. for C₁₀₄H₁₄₅N₂₇O₂₃: 2140.10, found: 1071.95 [M/2+H]⁺.

### Step 2: Synthesis of N-{5-[(2-{[2-({17-[(2S)-2-[(2S)-2-[(2S)-2-[(2S)-2-[(2S)-2-[(4-tert- butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl}carbamoyl)-1-methylimidazol-4-yl]carbamoyl}ethyl)carbamoyl]-1-methylpyrrol-3-yl}-4-[(2R)-2-acetamido-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-carboxamide (Comp. 109)

To a solution of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-2-({17-[(4-{3-[(4-{4-[(2R)-2-acetamido-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-amido}-1-methylpyrrol-2-yl)formamido]propanamido}-1-methylimidazol-2-yl)formamido]-3,6,9,12,15-pentaoxaheptadecan-1-yl}carbamoyl)ethyl acetate (25.00 mg, 0.01 mmol, 1.00 equiv) in MeOH (1.50 mL) was added 2 M LiOH (0.02 mL, 3.33 equiv). The resulting mixture was stirred at 45 degrees C. for 1.0 h. The resulting mixture was concentrated under reduced pressure. The residue was dissolved in H₂O (5 mL). The mixture was acidified to pH 3˜5 with 2 M HCl. The precipitated solids were collected by filtration and washed with H₂O (3×5 mL), dried under vacuum. The solids was dissolved in DMF (1.0 mL), the resulting mixture was filtered and the filtration in DMF (1.0 mL) was purified by Perp-HPLC: Column: XBridge Prep Phenyl OBD Column, 19*150 mm, 5 μm 13 nm; Mobile Phase A: Water (0.05% TFA), Mobile Phase B: ACN; Flow rate: 25 mL/min; Gradient: 40% B to 65% B in 15 min, 65% B; Wave Length: 254 n; RT1 (min): 9.35. The fractions were combined and lyophilized directly. N-{5-[(2-{[2-({17-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl}carbamoyl)-1-methylimidazol-4-yl]carbamoyl}ethyl)carbamoyl]-1-methylpyrrol-3-yl}-4-[(2R)-2-acetamido-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-carboxamide (3.70 mg, 14.83% yield) was obtained as light yellow solid. HRMS: mass calcd. for C₁₀₂H₁₄₃N₂₇O₂₂: 2098.0900, found: 2099.0925 [M+H]⁺.

### Example 35. Synthesis of Compound 120

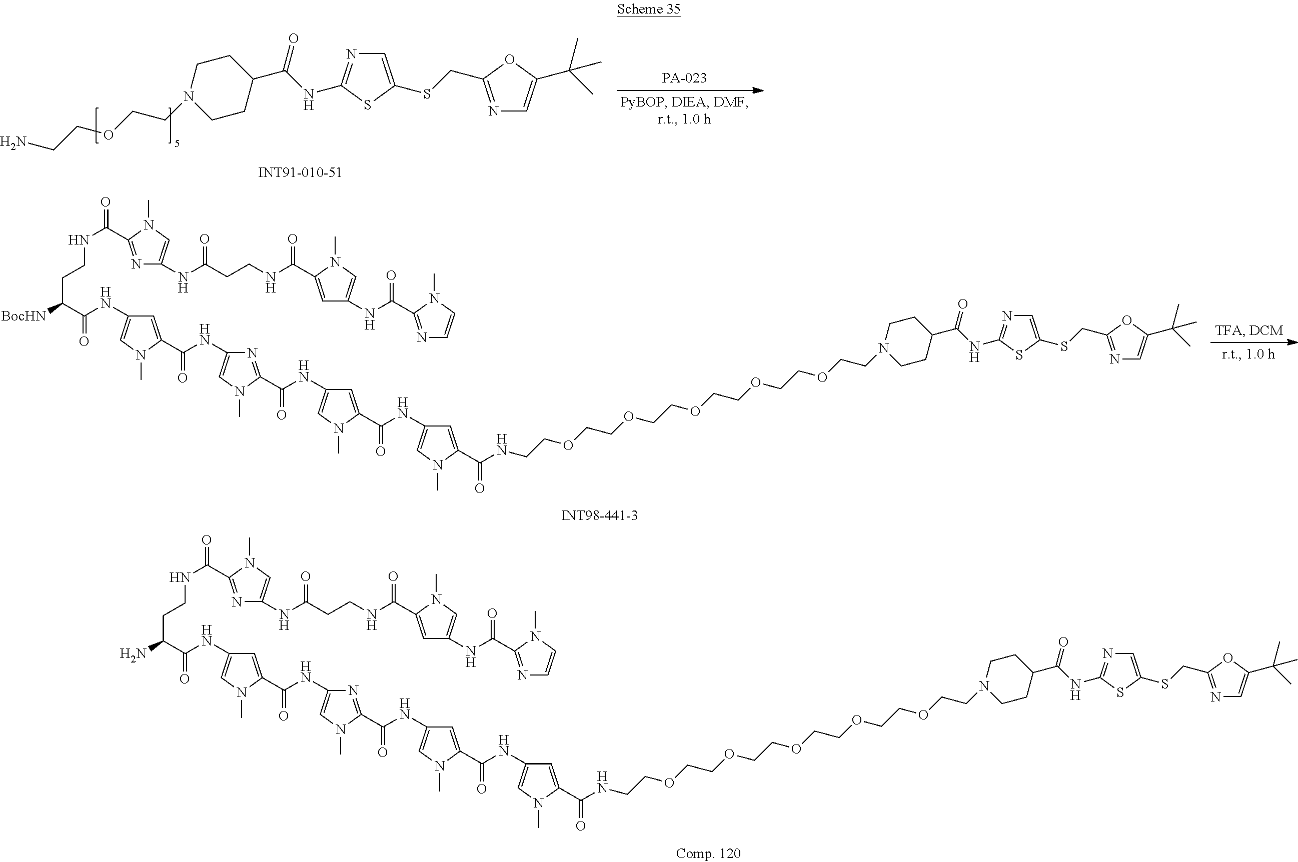

### Step 1: Synthesis of tert-butyl N-[(1S)-1-({5-[(2-{[5-({5-[(17-{4-[(5-{[(5-tert-butyl-1,3-oxazol-2-yl)methyl]sulfanyl}-1,3-thiazol-2-yl)carbamoyl]piperidin-1-yl}-3,6,9,12,15-pentaoxaheptadecan-1-yl)carbamoyl]-1-methylpyrrol-3-yl}carbamoyl)-1-methylpyrrol-3-yl]carbamoyl}-1-methylimidazol-4-yl)carbamoyl]-1-methylpyrrol-3-yl}carbamoyl)-3-{[1-methyl-4-(3-{[1-methyl-4-(1-methylimidazole-2-amido)pyrrol-2-yl]formamido}propanamido)imidazol-2-yl]formamido}propyl]carbamate (INT98-441-3)

The procedure was the same as (INT61-04-OH-21). 41.20 mg of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid was used, 35.00 mg of desired product was obtained as light yellow solid (49.20% yield). LC/MS: mass calcd. For C₈₀H₁₀₈N₂₄O₁₈S₂: 1756.77, found: 880.05 [M/2+H]⁺.

### Step 2: Synthesis of 1-[17-({4-[4-(4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrol-2-yl}formamido)-3,6,9,12,15-pentaoxaheptadecan-1-yl]-N-(5-{[(5-tert-butyl-1,3-oxazol-2-yl)methyl]sulfanyl}-1,3-thiazol-2-yl)piperidine-4-carboxamide (Comp. 120)

The procedure was the same as (INT-29-110), and the crude product was purified by Prep-HPLC. 30.00 mg of tert-butyl N-[(1S)-1-({5-[(2-{[5-({5-[(17-{4-[(5-{[(5-tert-butyl-1,3-oxazol-2-yl) methyl]sulfanyl}-1,3-thiazol-2-yl)carbamoyl]piperidin-1-yl}-3,6,9,12,15-pentaoxaheptadecan-1-yl)carbamoyl]-1-methylpyrrol-3-yl}carbamoyl)-1-methylpyrrol-3-yl]carbamoyl}-1-methylimidazol-4-yl)carbamoyl]-1-methylpyrrol-3-yl}carbamoyl)-3-{[1-methyl-4-(3-{[1-methyl-4-(1-methylimidazole-2-amido)pyrrol-2-yl]formamido}propanamido)imidazol-2-yl]formamido}propyl]carbamate was used, 9.60 mg of desired product was obtained as white solid (32.93% yield). HRMS: mass calcd. For C₇₅H₁₀₀N₂₄O₁₆S₂: 1656.7191, found: 1657.7342 [M+H]⁺.

### Example 36. Synthesis of Compound 123

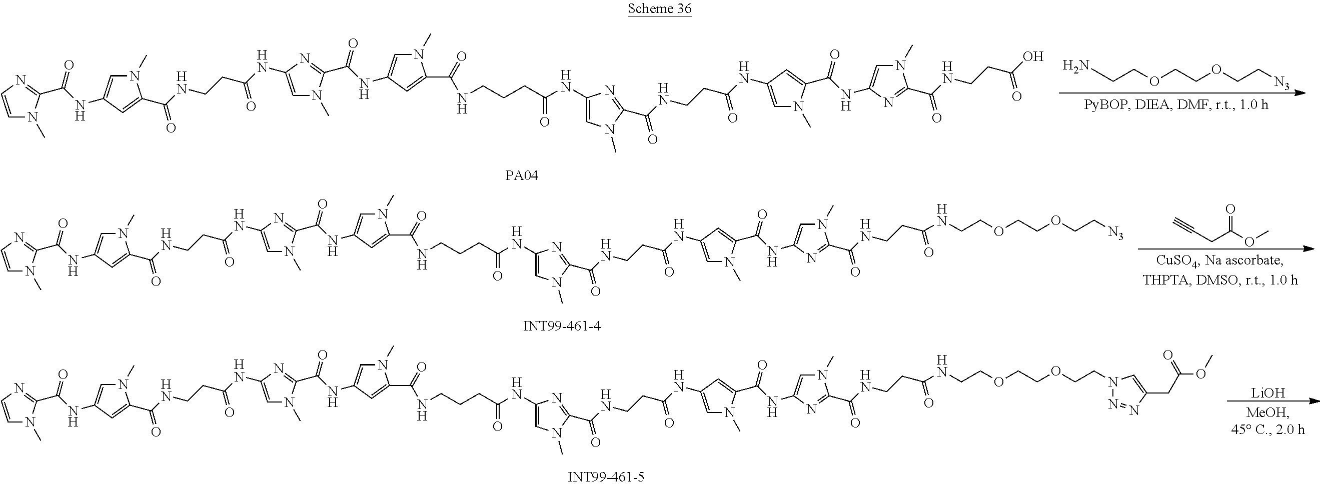

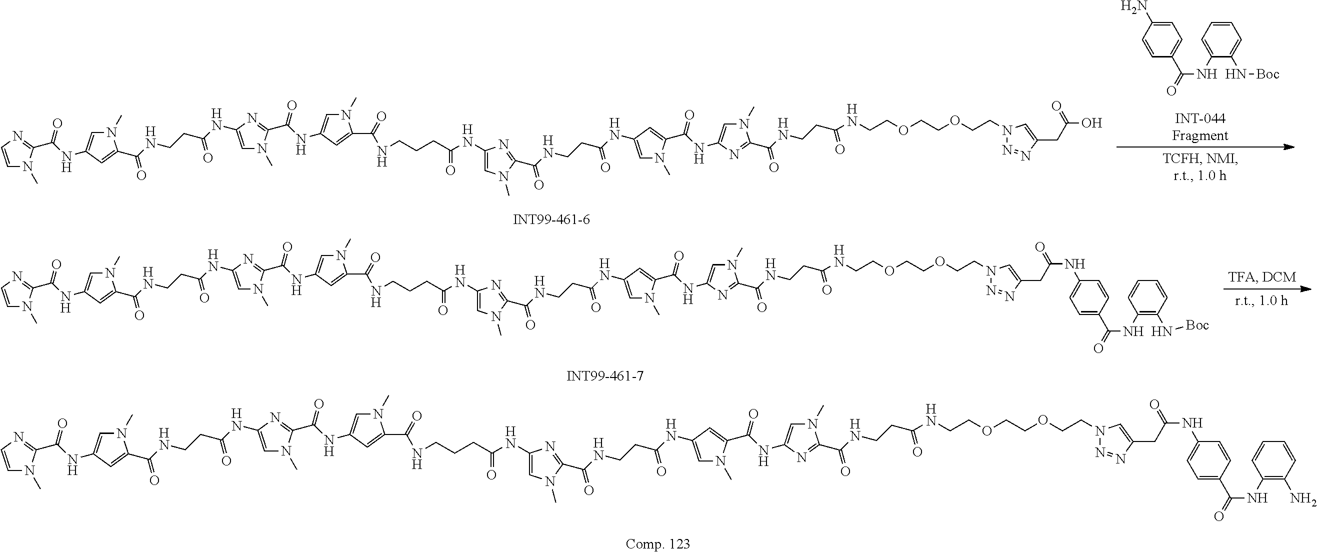

### Step 1: Synthesis of N-[5-({3-[(2-{[2-({5-[(2-{[2-({2-[2-(2-azidoethoxy) ethoxy]ethyl}carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (INT99-461-4)

The procedure was the same as (INT61-04-OH-21). 200.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 220.00 mg of desired product was obtained as yellow solid (87.25% yield). LC/MS: mass calcd. for C₅₇H₇₃N₂₅O₁₃: 1315.58, found: 659.35 [1/2 M+H]⁺.

### Step 2: Synthesis of methyl 2-(1-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetate (INT99-461-5)

To a solution of CuSO₄·5H₂O (4.36 mg, 0.02 mmol, 0.10 equiv), sodium ascorbate (17.39 mg, 0.09 mmol, 0.50 equiv), THPTA (379.59 mg, 0.88 mmol, 5.00 equiv) in DMSO (2.00 mL) was added N-[5-({3-[(2-{[2-({5-[(2-{[2-({2-[2-(2-azidoethoxy)ethoxy]ethyl}carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (230.00 mg, 0.18 mmol, 1.00 equiv), methyl but-3-ynoate (17.14 mg, 0.18 mmol, 1.00 equiv). The resulting mixture was stirred at room temperature for 1.0 h. The reaction mixture was purified by reverse flash chromatography with the following conditions: column, C18 silica gel; mobile phase, ACN in water (0.05% TFA), 35% to 50% gradient in 20 min; detector, UV 254 n. The fractions were combined and concentrated. The methyl 2-(1-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetate (150.00 mg, 60.69% yield) was obtained as white solid. LC/MS: mass calcd. for C₆₂H₇₉N₂₅O₁₅: 1413.61, found: 708.35 [1/2 M+H]⁺.

### Step 3: Synthesis of (1-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) butanamido]imidazol-2-yl}formamido)propanamido]pyrrole-2-amido}imidazol-2-yl)formamido]propanamido}ethoxy)ethoxy]ethyl}-1,2,3-triazol-4-yl)acetic acid (INT99-461-6)

The procedure was the same as 4-[3-[(Tert-butoxycarbonyl)amino] propanamido]-1-methylimidazole-2-carboxylic acid (INT60-022-2000). 140.00 mg of methyl 2-(1-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetate was used, 90.00 mg of desired product was obtained as yellow solid (64.93% yield). LC/MS: mass calcd. for C₆₁H₇₇N₂₅O₁₅: 1399.60, found: 701.35 [1/2M+H]⁺.

### Step 99-4: Synthesis of tert-butyl N-(2-{4-[2-(1-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetamido]benzamido}phenyl)carbamate (INT99-461-7)

The procedure was the same as N-(5-[[(5-tert-butyl-1,3-oxazol-2-yl) methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001, but the reaction time was 1.0 h. After the reaction, the reaction mixture was purified by reverse phase column. 70.00 mg of (1-{2-[2-(2-{3-[(1-methyl-4-{1-methyl-4-[3-({1-methyl-4-[4-({1-methyl-4-[1-methyl-4- (3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetic acid was used, 50.00 mg of desired product was obtained as yellow solid (58.50% yield). LC/MS: mass calcd. for C₇₉H₉₆N₂₈O₁₇: 1708.75, found: 856.05 [1/2M+H]⁺.

### Step 5: Synthesis of N-[5-({3-[(2-{[2-({5-[(2-{[2-({2-[2-(2-{4-[({4-[(2-aminophenyl)carbamoyl]phenyl}carbamoyl)methyl]-1,2,3-triazol-1-yl}ethoxy)ethoxy]ethyl}carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (Comp. 123)

The procedure was the same as (INT-29-110), but the crude was purified by Prep-HPLC. 46.00 mg of tert-butyl N-(2-{4-[2-(1-{2-[2-(2-{3-[(1-methyl-4-{1-methyl-4-[3-({1-methyl-4-[4-({1-methyl- 4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetamido]benzamido}phenyl)carbamate was used, 1.10 mg of desired product was obtained as white solid (2.35% yield). HRMS: mass calcd. for C₇₄H₈₈N₂₈O₁₅: 1608.6983, found: 1631.6928 [M+Na]⁺.

### Example 37. Synthesis of Compound 124

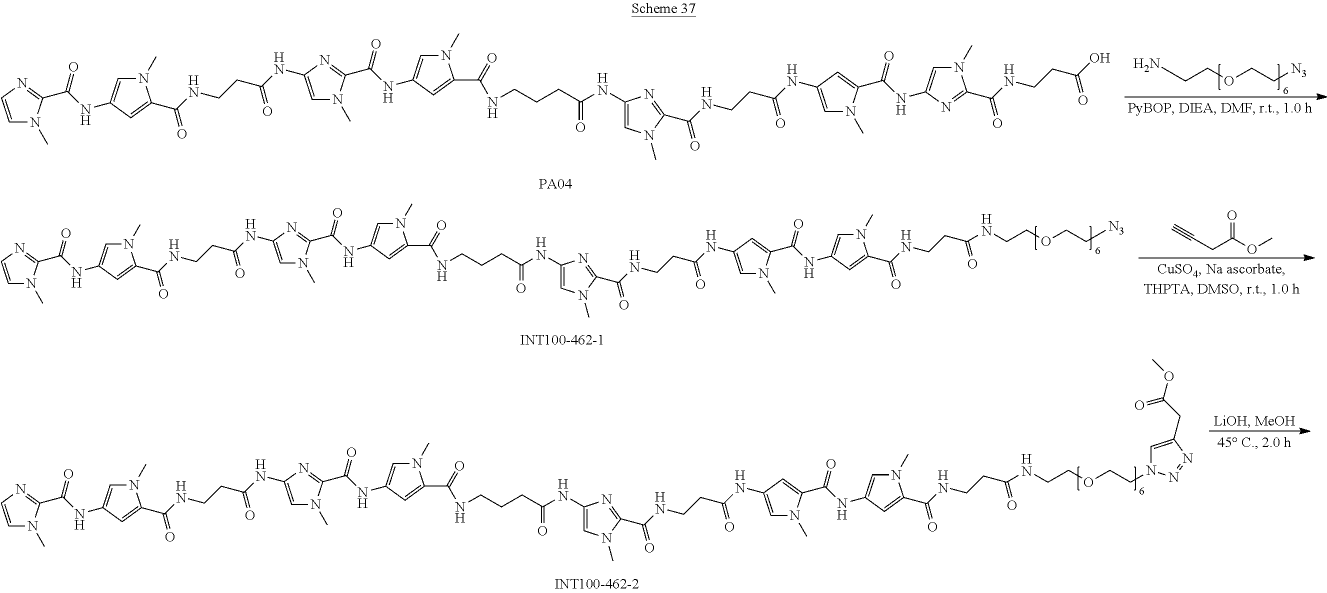

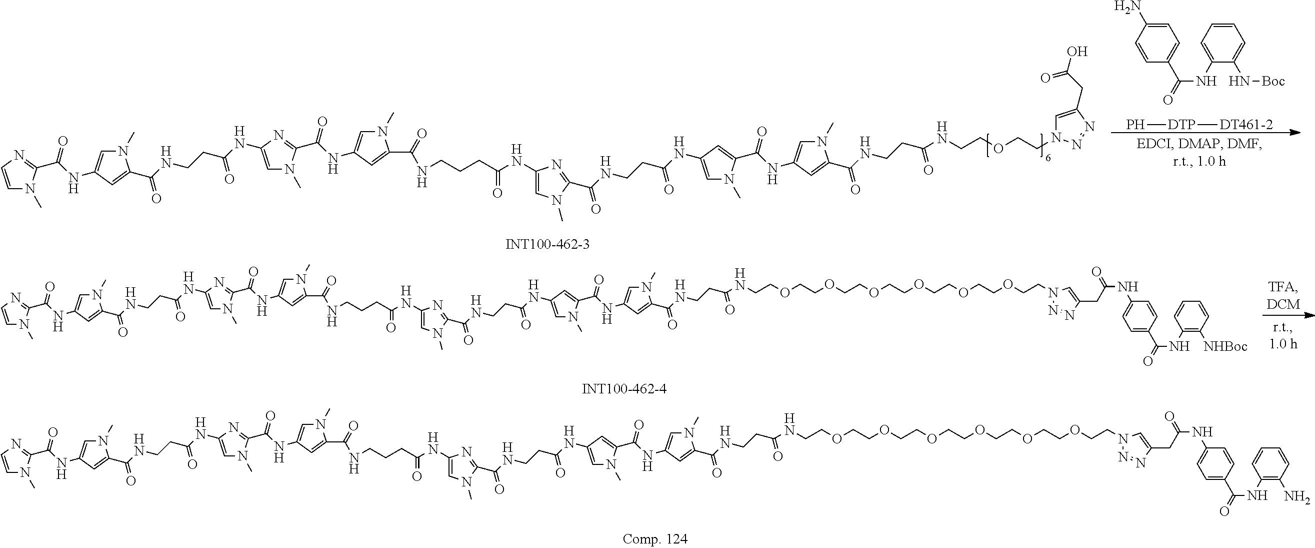

### Step 1: Synthesis of N-{5-[(3-{[2-({2-[(5-{[2-({2-[(20-azido-3,6,9,12,15,18-hexaoxaicosan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (INT100-462-1)

The procedure was the same as N (INT61-04-OH-21). After the reaction, the reaction mixture was poured into ice-water, the obtained solid could be used for next step directly. 300.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 340.00 mg of desired product was obtained as yellow solid (88.09% yield). LC/MS: mass calcd. for C₆₅H₈₉N₂₅O₁₇: 1491.68, found: 747.45 [M/2+H]⁺.

### Step 2: Synthesis of methyl 2-[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]acetate (INT100-462-2)

The procedure was the same as (INT99-461-5). 300.00 mg of N-{5-[(3-{[2-({2-[(5-{[2-({2-[(20-azido-3,6,9,12,15,18-hexaoxaicosan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide was used, 300.00 mg of desired product was obtained as white solid (93.83% yield). LC/MS: mass calcd. for C₇₀H₉₅N₂₅O₁₉: 1589.72, found: 796.50 [M/2+H]⁺.

### Step 3: Synthesis of [1-(20-{3-[(1-Methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]acetic acid (INT100-462-3)

The procedure was the same as 4-[3-[(Tert-butoxycarbonyl)amino] propanamido]-1-methylimidazole-2-carboxylic acid (INT60-022-2000). 300.00 mg of methyl 2-[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]acetate was used, 160.00 mg of desired product was obtained as white solid (53.80% yield). LC/MS: mass calcd. for C₆₉H₉₃N₂₅O₁₉: 1575.70, found: 789.50 [M/2+H]⁺.

### Step 4: Synthesis of tert-butyl N-[2-(4-{2-[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]acetamido}benzamido)phenyl]carbamate (INT100-462-4)

The procedure was the same as (INT61-025-20), but the reaction temperature was r.t., the reaction time was 1.0 h. After the reaction, the reaction mixture was purified by reverse phase column. 110.00 mg of [1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]acetic acid was used, 50.00 mg of desired product was obtained as white solid (38.00% yield). LC/MS: mass calcd. for C₈₇H₁₁₂N₂₈O₂₁: 1884.85, found: 944.20 [M/2+H]⁺.

### Step 5: Synthesis of N-{5-[(3-{[2-({2-[(5-{[2-({2-[(20-{4-[({4-[(2-aminophenyl) carbamoyl]phenyl}carbamoyl)methyl]-1,2,3-triazol-1-yl}-3,6,9,12,15,18-hexaoxaicosan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 124)

The procedure was the same as (INT-29-110), but the crude product was purified by Prep-HPLC. 41.00 mg of tert-butyl N-[2-(4-{2-[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]acetamido}benzamido)phenyl]carbamate was used, 6.40 mg of desired product was obtained as white solid (16.13% yield). HRMS: mass calcd. for C₈₂H₁₀₄N₂₈O₁₉: 1784.8032, found: 1785.8050 [M+H]⁺.

### Example 38. Synthesis of Synthesis of Compound 128

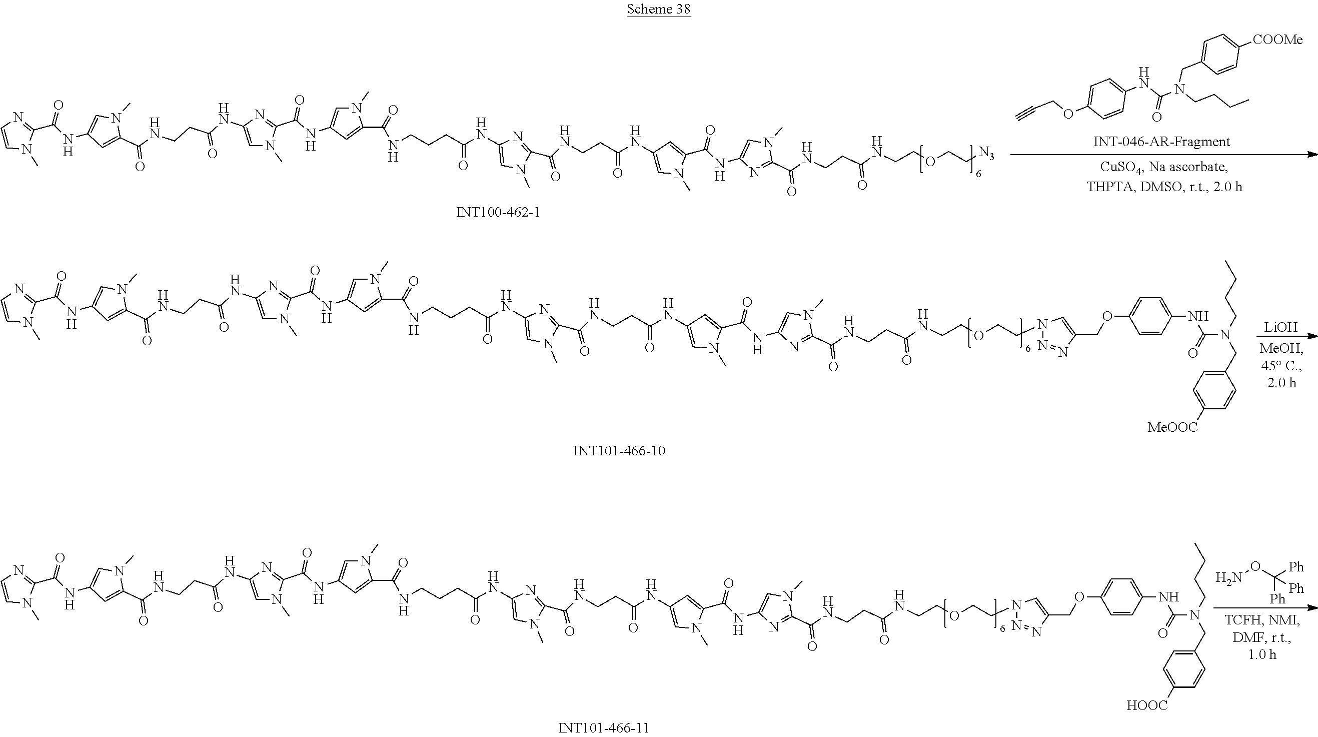

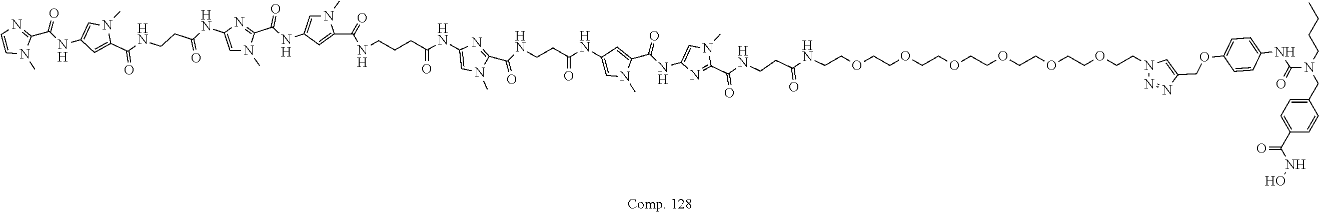 [SMILES: CCCCN(Cc1ccc(C(=O)NO)cc1)C(=O)Nc1ccc(OCc2cn(CCOCCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)nn2)cc1]

### Step 1: Synthesis of methyl 4-({butyl[(4-{[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]methoxy}phenyl)carbamoyl]amino}methyl)benzoate (INT101-466-10)

The procedure was the same as methyl 2-(1-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}-1,2,3-triazol-4-yl)acetate (INT99-461-5), but the reaction time was 2.0 h. 115.00 mg of N-{5-[(3-{[2-({2-[(5-{[2-({2-[(20-azido-3,6,9,12,15,18-hexaoxaicosan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide was used, 110.00 mg of desired product was obtained as light brown solid (75.66% yield). LC/MS: mass calcd. For C₈₈H₁₁₅N₂₇O₂₁: 1885.87, found: 944.30 [M/2+H]⁺.

### Step 2: Synthesis of 4-({butyl[(4-{[1-(20-{3-[(1-methyl-4-{1-methyl-4-[3-{[1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]methoxy}phenyl)carbamoyl]amino}methyl)benzoic acid (INT101-466-11)

The procedure was the same as 4-[3-[(Tert-butoxycarbonyl)amino] propanamido]-1-methylimidazole-2-carboxylic acid (INT60-022-2000), but the solvent was THF/MeOH. 110.00 mg of methyl 4-({butyl[(4-{[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]methoxy}phenyl)carbamoyl]amino}methyl)benzoate was used, 84.00 mg of desired product was obtained as dark green solid (76.94% yield). LC/MS: mass calcd. For C₈₇H₁₁₃N₂₇O₂₁: 1871.86, found: 937.75[M/2+H]⁺.

### Step 3: Synthesis of N-[5-({3-[(2-{[2-([5-({2-{[2-({20-[4-(4-{[butyl({[4-(hydroxycarbamoyl)phenyl]methyl})carbamoyl]amino}phenoxymethyl)-1,2,3-triazol-1-yl]-3,6,9,12,15,18-hexaoxaicosan-1-yl}carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (Comp. 128)

The procedure was the same as N-(5-[[(5-tert-butyl-1,3-oxazol-2-yl) methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001). 75.00 mg of 4-({butyl[(4-{[1-(20-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15,18-hexaoxaicosan-1-yl)-1,2,3-triazol-4-yl]methoxy}phenyl)carbamoyl]amino}methyl)benzoic acid was used, 2.80 mg of desired product was obtained as white solid (3.53% yield). HRMS: mass calcd. For C₈₇H₁₁₄N₂₈O₂₁: 1886.8713, found: 1887.8745 [M+H]⁺.

### Example 39. Synthesis of Compound 074

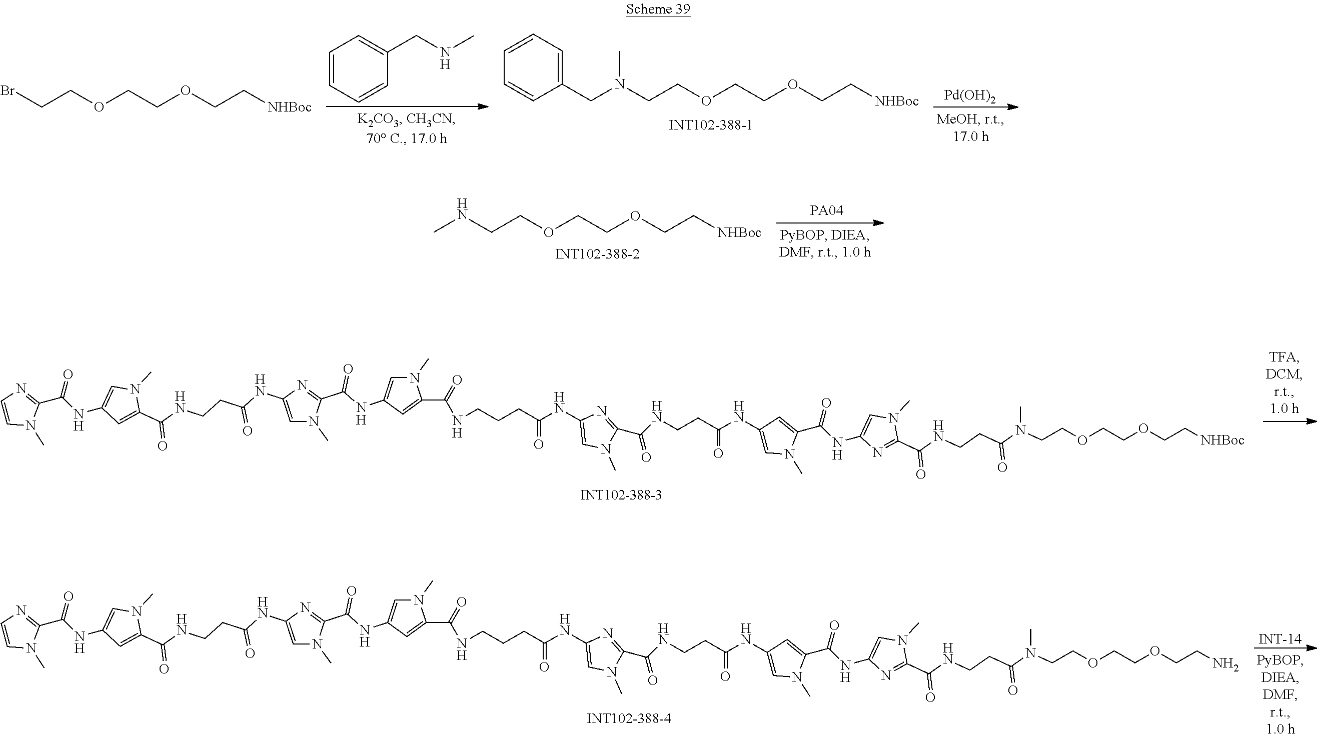

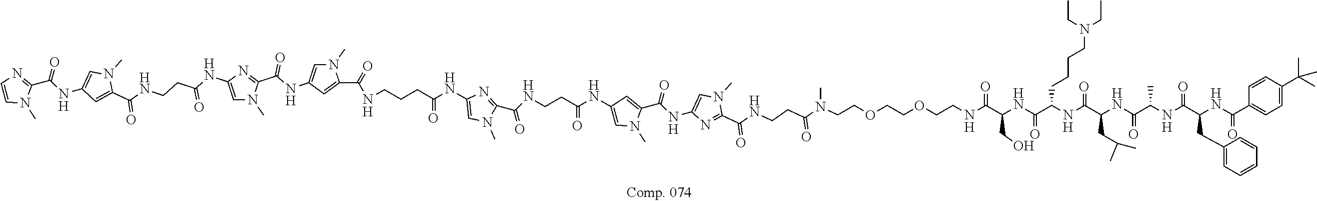 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCOCCOCCN(C)C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C]

### Step 1: Synthesis of tert-butyl N-[2-(2-{2-[benzyl(methyl)amino]ethoxy}ethoxy) ethyl]carbamate (INT102-388-1)

To a solution of tert-butyl N-{2-[2-(2-bromoethoxy)ethoxy]ethyl}carbamate (1.00 g, 3.203 mmol, 1.00 equiv) in ACN (10.00 mL) was added K₂CO₃ (1.33 g, 9.609 mmol, 3.00 equiv) and N-methylbenzylamine (0.78 g, 6.406 mmol, 2.00 equiv). Then the reaction was stirred at 70 degrees C. overnight. The suspension was filtered and the filtrate was purified by silica gel column chromatography, eluted with CH₂Cl₂/MeOH (15:01) to afford tert-butyl N-[2-(2-{2-[benzyl(methyl)amino]ethoxy}ethoxy)ethyl]carbamate (1.00 g, 88.57%) as a light yellow oil. LC/MS: mass calcd. For C₁₉H₃₂N₂O₄: 352.24 found: 353.15 [M+H]⁺.

### Step 2: Synthesis of tert-butyl N-(2-{2-[2-(methylamino)ethoxy]ethoxy} ethyl)carbamate (INT102-388-2)

To a solution of tert-butyl N-[2-(2-{2-[benzyl(methyl)amino]ethoxy}ethoxy)ethyl]carbamate (160.00 mg, 0.45 mmol, 1.00 equiv) in MeOH (3.00 mL) was added Pd(OH)₂/C (40.00 mg, 25% w/w). Then the reaction was stirred for 17.0 h at room temperature under H₂ atmosphere. The mixture was filtrated and the filtrate was concentrated to afford tert-butyl N-(2-{2-[2-(methylamino)ethoxy]ethoxy}ethyl)carbamate (100.00 mg, crude) as a light yellow oil. LC/MS: mass calcd. For C₁₂H₂₆N₂O₄: 262.19 found: 263.20 [M+H]⁺.

### Step 3: Synthesis of tert-butyl N-{2-[2-(2-{N-methyl-3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}carbamate (INT102-388-3)

The procedure was the same as (INT61-04-OH-21). 79.60 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 60.00 mg of desired product was obtained as white solid (56.04% yield). LC/MS: mass calcd. For C₆₃H₈₅N₂₃O₁₅: 1403.66 found: 703.35 [M/2+H]⁺.

### Step 4: Synthesis of N-[5-({3-[(2-{[2-({5-[(2-{[2-({2-[2-(2-aminoethoxy) ethoxy]ethyl}(methyl)carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (INT102-388-4)

The procedure was the same as (INT-29-110). 50.00 mg of tert-butyl N-{2-[2-(2-{N-methyl-3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}carbamate was used, 50.00 mg crude of desired product was obtained as yellow oil. LC/MS: mass calcd. For C₅₈H₇₇N₂₃O₁₃: 1303.61 found: 1304.80 [M+H]⁺.

### Step 5: Synthesis of N-(5-{[3-({2-[(2-{[5-({2-[(2-{[2-(2-{2-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]ethoxy}ethoxy)ethyl](methyl)carbamoyl}ethyl)carbamoyl]-1-methylimidazol-4-yl}carbamoyl)-1-methylpyrrol-3-yl]carbamoyl}ethyl)carbamoyl]-1-methylimidazol-4-yl}carbamoyl)propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Comp. 074)

The procedure was the same as (INT61-06-OH-21), but the reaction mixture was purified by Prep-HPLC. 30.00 mg of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoic acid was used, 10.60 mg of desired product was obtained as white solid (11.92% yield). HRMS: mass calcd. For C₁₀₀H₁₃₉N₂₉O₂₀: 2066.0751 found: 2067.0925 [M+H]⁺.

### Example 40. Synthesis of Compound 129

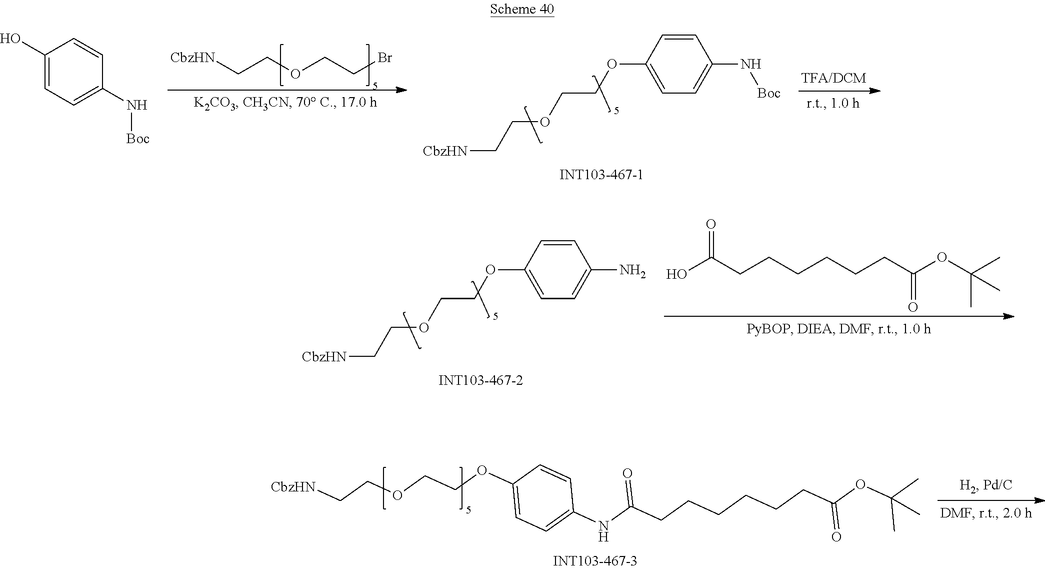

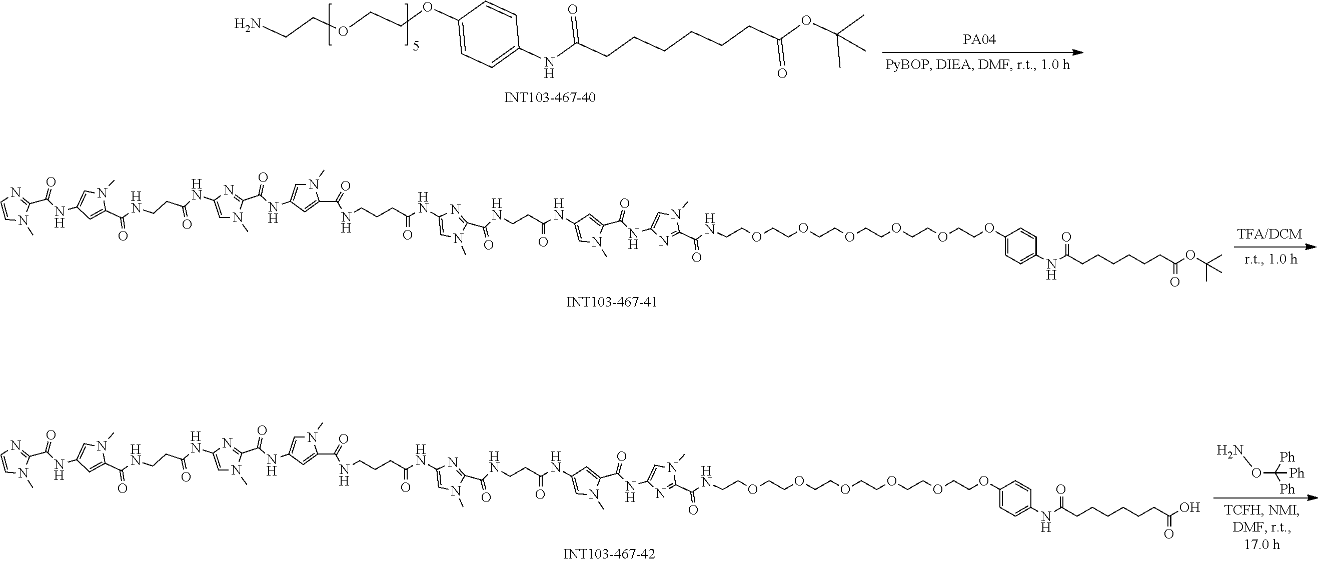

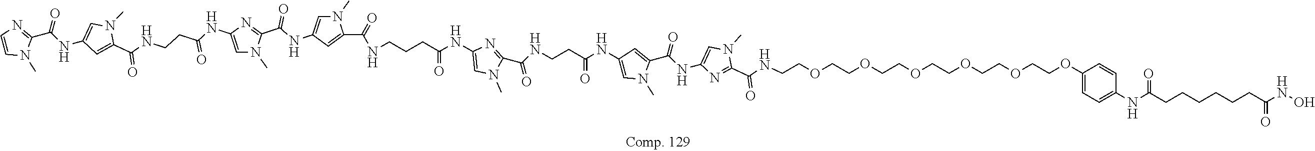 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCOCCOCCOCCOCCOCCOc4ccc(NC(=O)CCCCCCC(=O)NO)cc4)n3)n(C)c2)n1]

### Step 1: Synthesis of benzyl N-(17-{4-[(tert-butoxycarbonyl)amino]phenoxy}-3,6,9,12,15-pentaoxaheptadecan-1-yl)carbamate (INT103-467-1)

The procedure was the same as tert-butyl N-[2-(2-{2-[benzyl(methyl) amino]ethoxy}ethoxy)ethyl]carbamate (INT102-388-1). 1.00 mg of tert-butyl N-(4-hydroxyphenyl)carbamate was used, 150.00 mg of desired product was obtained as yellow oil (68.98% yield). LC/MS: mass calcd. For C₃₁H₄₆N₂O₁₀: 606.32, found: 607.60 [M+H]⁺.

### Step 2: Synthesis of benzyl N-[17-(4-aminophenoxy)-3,6,9,12,15-pentaoxaheptadecan-1-yl]carbamate (INT103-467-2)

The procedure was the same as (INT-29-110). 140.00 mg of benzyl N-(17-{4-[(tert-butoxycarbonyl)amino] phenoxy}-3,6,9,12,15-pentaoxaheptadecan-1-yl)carbamate was used, 140.00 mg crude of desired product was obtained yellow oil. LC/MS: mass calcd. For C₂₆H₃₈N₂O₈: 506.26, found: 507.30 [M+H]⁺.

### Step 3: Synthesis of tert-butyl 7-({4-[(17-{[(benzyloxy)carbonyl]amino}-3,6,9,12,15-pentaoxaheptadecan-1-yl)oxy]phenyl}carbamoyl)heptanoate (INT103-467-3)

The procedure was the same as tert-butyl N-[2-(2-{2-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]ethoxy}ethoxy)ethyl]carbamate (INT61-04-OH-21). 64.00 mg of 8-(tert-butoxy)-8-oxooctanoic acid was used, 110.00 mg of desired product was obtained as yellow oil (55.37% yield). LC/MS: mass calcd. For C₃₈H₅₈N₂O₁₁: 718.40, found: 719.45 [M+H]⁺.

### Step 4: Synthesis of tert-butyl 7-({4-[(17-amino-3,6,9,12,15-pentaoxaheptadecan-1-yl)oxy]phenyl}carbamoyl)heptanoate (INT103-467-40)

To a solution of tert-butyl 7-({4-[(17-{[(benzyloxy)carbonyl]amino}-3,6,9,12,15-pentaoxaheptadecan-1-yl)oxy]phenyl}carbamoyl)heptanoate (85.00 mg, 0.12 mmol, 1.00 equiv) in DMF (2.00 mL) was added Pd/C (17.00 mg, 20% w/w). Then H2 was exchanged by three times. The mixture was stirred for 2.0 h at room temperature. The Pd/C was filtered out and washed by MeOH, the filtration was concentrated and lyophilized. Tert-butyl 7-({4-[(17-amino-3,6,9,12,15-pentaoxaheptadecan-1-yl)oxy]phenyl}carbamoyl)heptanoate (70.00 mg, crude) was obtained as a yellow oil. LC/MS: mass calcd. For C₃₀H₅₂N₂O₉: 584.37, found: 585.40[M+H]⁺.

### Step 5: Synthesis of tert-butyl 7-({4-[(17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecan-1-yl)oxy]phenyl}carbamoyl)heptanoate (INT103-467-41)

The procedure was the same as (INT61-04-OH-21). 117.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 140.00 mg of desired product was obtained as yellow solid (72.93% yield). LC/MS: mass calcd. For C₈₁H₁₁₁N₂₃O₂₀: 1725.84, found: 864.50 [M/2+H]⁺.

### Step 6: Synthesis of 7-({4-[(17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecan-1-yl)oxy]phenyl}carbamoyl)heptanoic acid (INT103-467-42)

The procedure was the same as (INT-29-110). 50.00 mg of tert-butyl 7-({4-[(17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecan-1-yl)oxy]phenyl}carbamoyl)heptanoate was used, 50.00 mg crude of desired product was obtained as yellow oil. LC/MS: mass calcd. For C₇₇H₁₀₃N₂₃O₂₀: 1669.77, found: 836.45 [M/2+H]⁺.

### Step 7: Synthesis of N-hydroxy-N′-{4-[(17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecan-1-yl)oxy]phenyl}octanediamide (Comp. 129)

The procedure was the same as N-(5-[[(5-tert-butyl-1,3-oxazol-2-yl)methyl] sulfanyl]-1,3-thiazol-2-yl)-1-[17-([1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-yl]formamido)-3,6,9,12,15-pentaoxaheptadecan-1-yl]piperidine-4-carboxamide (Comp. 001), but the reaction time was 17.0 h. 50.00 mg of 7-({4-[(17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecan-1-yl)oxy]phenyl}carbamoyl)heptanoic acid was used, 8.50 mg of desired product was obtained as white solid (16.69% yield). HRMS: mass calcd. For C₇₇H₁₀₄N₂₄O₂₀: 1684.7859, found: 1685.7944 [M+H]⁺.

### Example 41. Synthesis of Compound 142

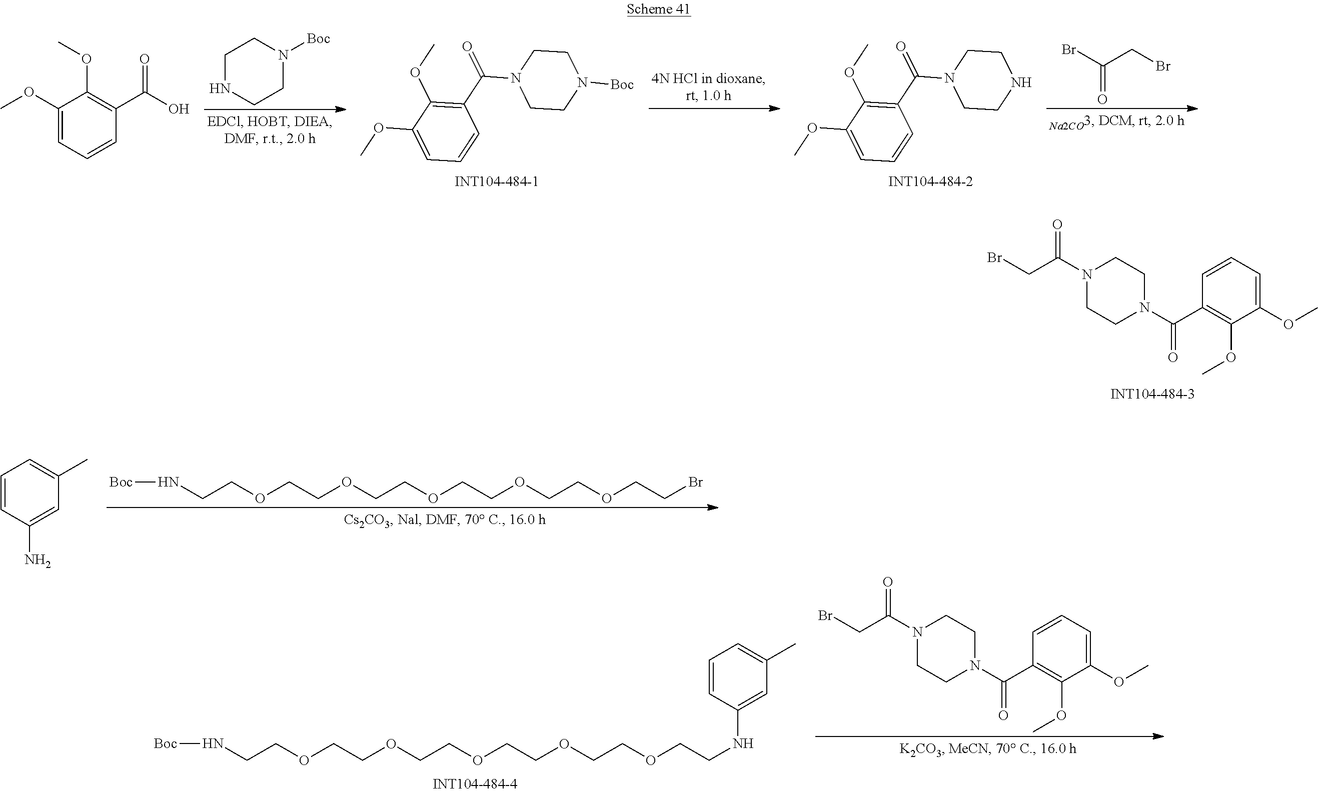

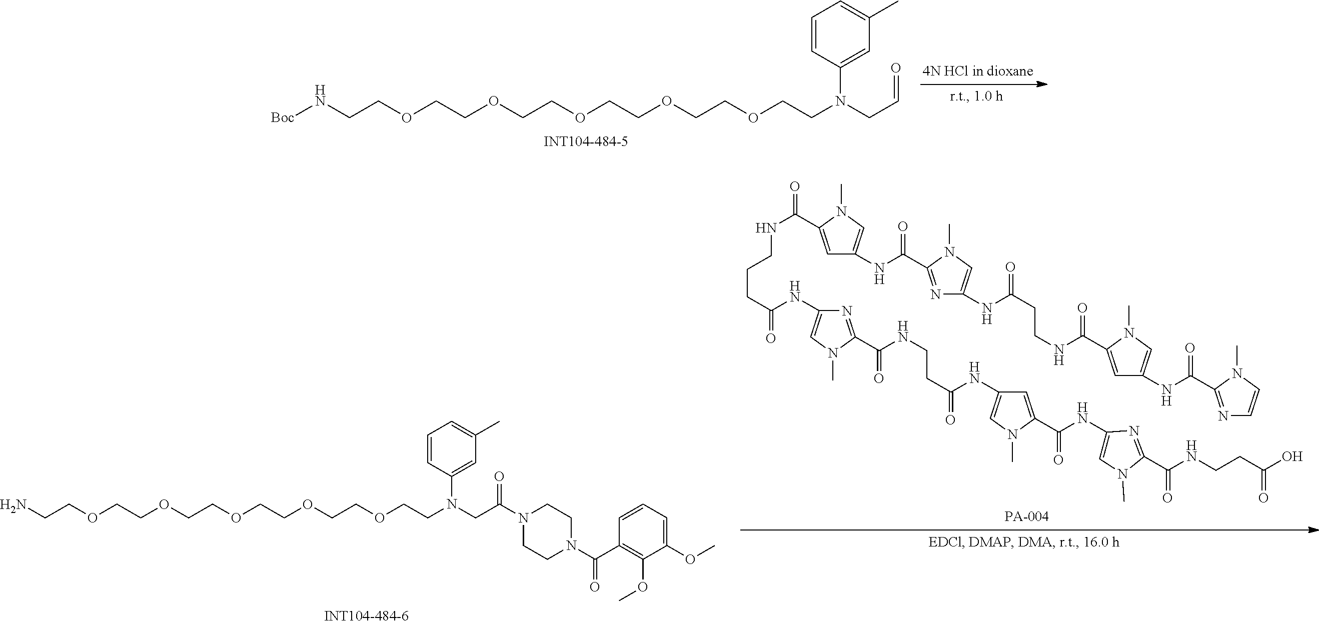 [SMILES: Cc1cccc(N(CC=O)CCOCCOCCOCCOCCOCCNC(=O)OC(C)(C)C)c1 ; COc1cccc(C(=O)N2CCN(C(=O)CN(CCOCCOCCOCCOCCOCCN)c3cccc(C)c3)CC2)c1OC ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

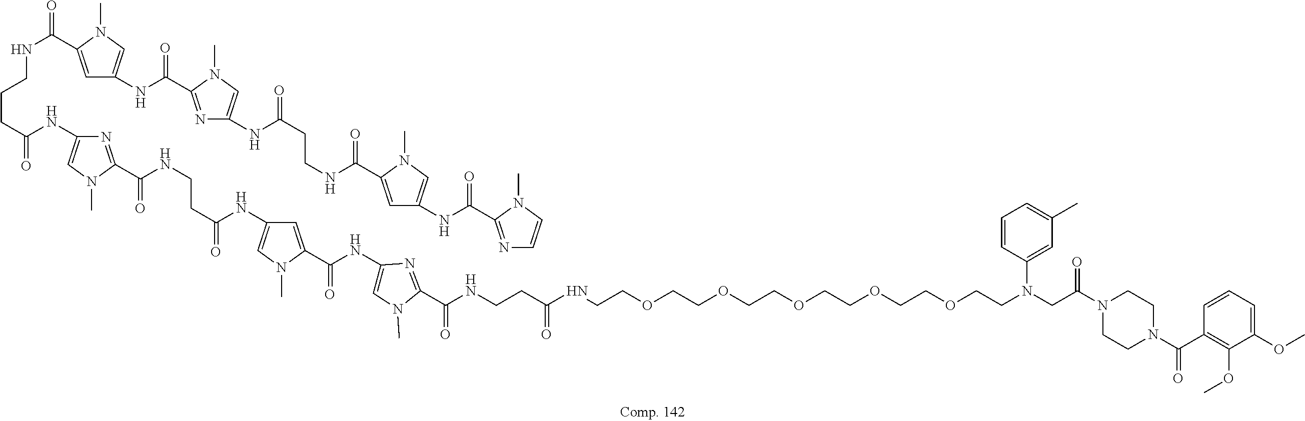 [SMILES: COc1cccc(C(=O)N2CCN(C(=O)CN(CCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)c3cccc(C)c3)CC2)c1OC]

### Step 1: Synthesis of tert-butyl 4-(2,3-dimethoxybenzoyl)piper azine-1-carboxylate (INT104-484-1)

To the mixture of 2,3-dimethoxybenzoic acid (2.00 g, 10.98 mmol, 1.00 equiv) and tert-butyl piperazine-1-carboxylate (2.25 g, 12.08 mmol, 1.10 equiv) in DMF (20.00 mL, 258.44 mmol, 23.54 equiv) was added EDCI (3.16 g, 16.47 mmol, 1.50 equiv), HOBT (2.23 g, 16.47 mmol, 1.50 equiv) and DIEA (3.55 g, 27.45 mmol, 2.50 equiv). The mixture was stirred at rt for 2.0 h. To the mixture was added H₂O (20 mL) and extracted by EtOAc (30 mL×3). The organic layer was combined and washed by sat. aq. critic acid (50 mL×2), sat. aq. NaHCO₃ (50 mL×2), brine (50 mL), dried over Na₂SO₄, filtered out and concentrated under reduced pressure. 3.50 g of desired product was obtained as a white solid (90.98% yield). LC/MS: mass calcd. For C₁₈H₂₆N₂O₅: 350.18, found: 351.10 [M+H]⁺.

### Step 2: Synthesis of 1-(2,3-dimethoxybenzoyl)piperazine (INT104-484-2)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51). 4.00 g of tert-butyl 4-(2,3-dimethoxybenzoyl)piperazine-1-carboxylate was used, 2.80 g of desired product was obtained as a white solid (98.00% yield). LC/MS: mass calcd. For C₁₃H₁₈N₂O₃: 250.13, found: 251.10 [M+H]⁺.

### Step 3: Synthesis of 2-bromo-1-[4-(2,3-dimethoxybenzoyl)piperazin-1-yl]ethenone (INT104-484-3)

To the mixture of 1-(2,3-dimethoxybenzoyl)piperazine (2.80 g, 11.19 mmol, 1.00 equiv) and bromoacetyl bromide (2.48 g, 12.31 mmol, 1.10 equiv) in CH₂Cl₂ (40.00 mL, 629.27 mmol, 56.25 equiv) was added Na₂CO₃ (3.56 g, 33.56 mmol, 3.00 equiv) in H₂O (40.00 mL, 2220.40 mmol, 198.48 equiv). The mixture was stirred at r.t. 2.0 h. The organic layer was washed by 5% HCl (50 mL×2), H₂O (50 mL×2), brine (50 mL×2) and dried over Na₂SO₄, filtered and concentrated under reduced pressure. The crude product was purified by flash chromatography (DCM:MeOH=10:1). 3.00 g of desired product was obtained as a white solid (72.24% yield). LC/MS: mass calcd. For C₁₅H₁₉BrN₂O₄: 370.05, found: 371.00 [M+H]⁺.

### Step 4: Synthesis of tert-butyl N-{17-[(3-methylphenyl)amino]-3,6,9,12,15-pentaoxaheptadecan-1-yl}carbamate (INT104-484-4)

To the mixture of M-toluidine (50.00 mg, 0.47 mmol, 1.00 equiv) and tert-butyl N-(17-bromo-3,6,9,12,15-pentaoxaheptadecan-1-yl)carbamate (207.34 mg, 0.47 mmol, 1.00 equiv) in DMF (3.00 mL, 38.77 mmol, 83.08 equiv) was added NaI (69.94 mg, 0.47 mmol, 1.00 equiv) and Cs₂CO₃ (456.09 mg, 1.40 mmol, 3.00 equiv). The mixture was stirred at 80° C. for 16.0 h. The residue was purified by reverse flash chromatography with the following conditions: column, silica gel; mobile phase, MeCN in water (0.05% TFA), 10% to 60% gradient in 20 min; detector, UV 254 n. 70.00 mg of desired product was obtained as colorless oil (31.88% yield). LC/MS: mass calcd. For C₂₄H₄₂N₂O₇: 470.30, found: 471.25 [M+H]⁺.

### Step 5: Synthesis of tert-butyl N-{20-[4-(2,3-dimethoxybenzoyl)piperazin-1-yl]-18-(3-methylphenyl)-20-oxo-3,6,9,12,15-pentaoxa-18-azaicosan-1-yl}carbamate (INT104-484-5)

The procedure was the same as tert-butyl N-[2-(2-{2-[benzyl(methyl) amino]ethoxy}ethoxy)ethyl]carbamate (INT102-388-1). After the reaction, the reaction mixture was filtered and the filtrated was concentrated. The residue was purified Prep-TLC. 70.00 mg of N-{17-[(3-methylphenyl)amino]-3,6,9,12,15-pentaoxaheptadecan-1-yl}carbamate was used, 80.00 mg of desired product was obtained as a white solid (70.68% yield). LC/MS: mass calcd. For C₃₉H₆₀N₄O₁₁: 760.43, found: 761.40 [M+H]⁺.

### Step 6: Synthesis of 1-amino-20-[4-(2,3-dimethoxybenzoyl)piperazin-1-yl]-18-(3-methylphenyl)-3,6,9,12,15-pentaoxa-18-azaicosan-20-one (INT104-484-6)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51). But the reaction time was 1 h. 90.00 mg of tert-butyl N-{20-[4-(2,3-dimethoxybenzoyl)piperazin-1-yl]-18-(3-methylphenyl)-20-oxo-3,6,9,12,15-pentaoxa-18-azaicosan-1-yl}carbamate was used, 70.00 mg of desired product was obtained as colorless oil (89.56% yield). LC/MS: mass calcd. For C₃₄H₅₂N₄O₉: 660.37, found: 661.35 [M+H]⁺.

### Step 7: Synthesis of N-[5-({3-[(2-{[2-({5-[(2-{[2-({20-[4-(2,3-dimethoxybenzoyl)piperazin-1-yl]-18-(3-methylphenyl)-20-oxo-3,6,9,12,15-pentaoxa-18-azaicosan-1-yl}carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (Comp. 142)

The procedure was the same as (INT61-025-20), but the reaction temperature was r.t., the reaction time was 16 h. 30.00 mg of 1-amino-20-[4-(2,3-dimethoxybenzoyl)piperazin-1-yl]-18-(3-methylphenyl)-3,6,9,12,15-pentaoxa-18-azaicosan-20-one was used, 11.50 mg of desired product was obtained as a white solid (13.50% yield). LC/MS: mass calcd. For C₈₅H₁₁₁N₂₅O₂₀: 1801.8437, found: 1824.8310 [M+Na]⁺.

### Example 42. Synthesis of Compound 143

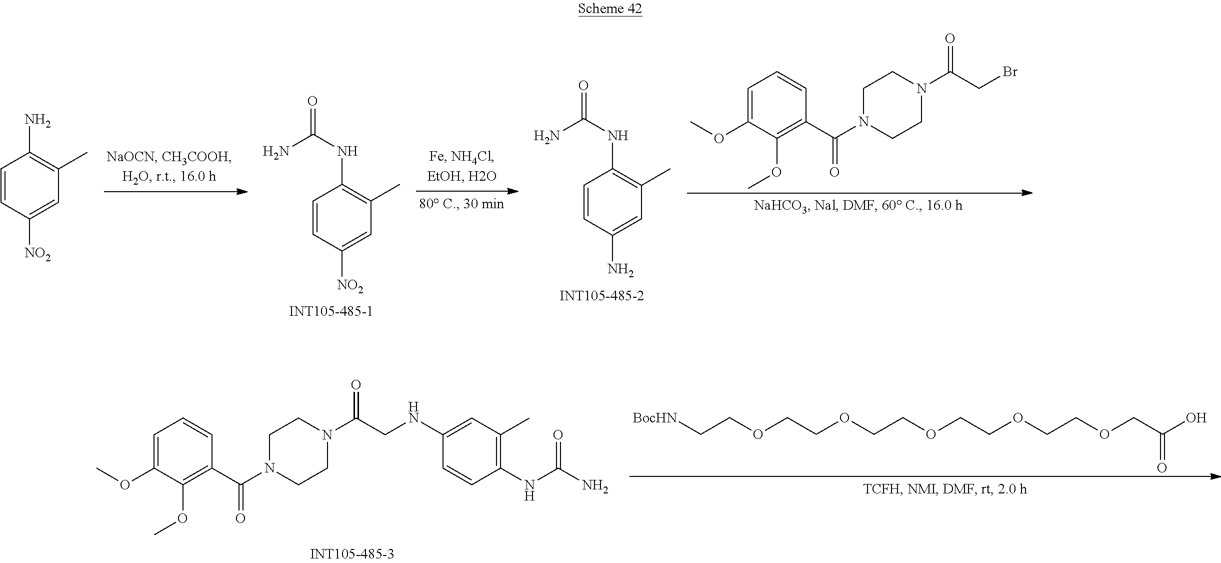 [SMILES: Cc1cc([N+](=O)[O-])ccc1N ; Cc1cc([N+](=O)[O-])ccc1NC(N)=O ; Cc1cc(N)ccc1NC(N)=O ; COc1cccc(C(=O)N2CCN(C(=O)CBr)CC2)c1OC ; COc1cccc(C(=O)N2CCN(C(=O)CNc3ccc(NC(N)=O)c(C)c3)CC2)c1OC ; CC(C)(C)OC(=O)NCCOCCOCCOCCOCCOCC(=O)O]

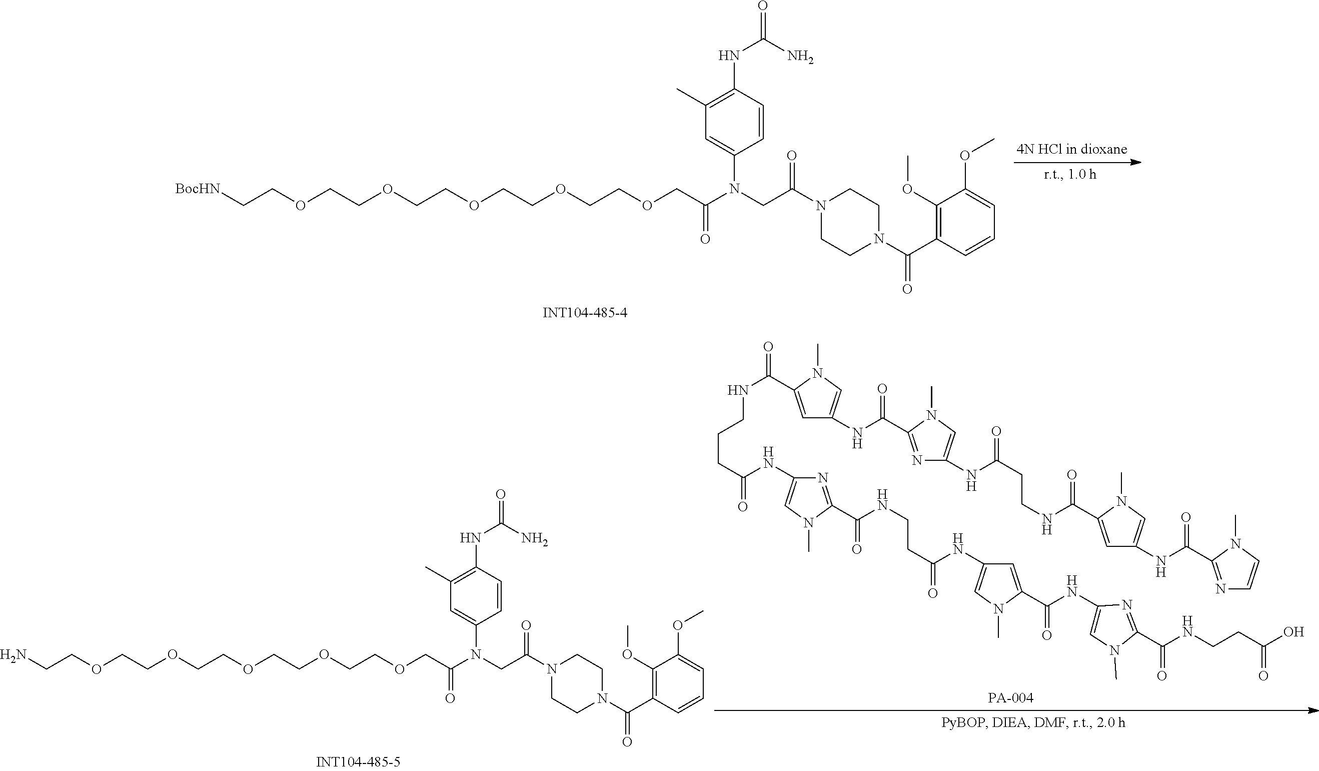 [SMILES: COc1cccc(C(=O)N2CCN(C(=O)CN(C(=O)COCCOCCOCCOCCOCCNC(=O)OC(C)(C)C)c3ccc(NC(N)=O)c(C)c3)CC2)c1OC ; COc1cccc(C(=O)N2CCN(C(=O)CN(C(=O)COCCOCCOCCOCCOCCN)c3ccc(NC(N)=O)c(C)c3)CC2)c1OC ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

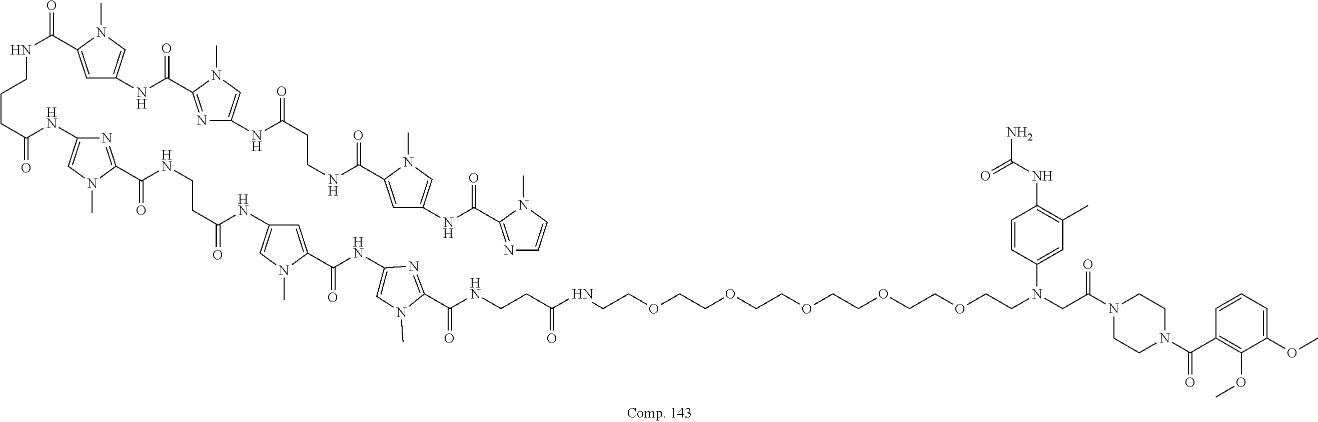 [SMILES: COc1cccc(C(=O)N2CCN(C(=O)CN(CCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)c3ccc(NC(N)=O)c(C)c3)CC2)c1OC]

### Step 1: Synthesis of 2-methyl-4-nitrophenylurea (INT105-485-1)

To the mixture of 4-nitro-2-toluidine (2.00 g, 13.15 mmol, 1.00 equiv) in CH₃COOH (20.00 mL) was added Sodium cyanate (1.79 g, 27.61 mmol, 2.10 equiv) in H₂O (15.00 mL). The mixture was stirred at r.t. for 16.0 h. The precipitated solids were collected by filtration and washed with Et₂O (3×10 mL). 700.00 mg of desired product was obtained as an off-white solid. LC/MS: mass calcd. For C₈H₉N₃O₃: 195.06, found: 196.10 [M+H]⁺.

### Step 2: Synthesis of 4-amino-2-methylphenylurea (INT105-485-2)

To the mixture of 2-methyl-4-nitrophenylurea (700.00 mg, 3.59 mmol, 1.00 equiv) and NH₄Cl (1918.44 mg, 35.86 mmol, 10.00 equiv) in EtOH (15.00 mL) and H₂O (10.00 mL) was added Fe (2002.86 mg, 35.86 mmol, 10.00 equiv) slowly at 80° C. The mixture was stirred at the same temperature for 30 min. The resulting mixture was filtered, the filter cake was washed with EtOAc. The filtrate was concentrated under reduced pressure. The residue was dissolved in EtOAc (30 mL). The resulting mixture was filtered, the filter cake was washed with EtOAc. The filtrate was concentrated under reduced pressure. 200.00 mg of desired product was obtained as a brown yellow solid (33.76% yield). LC/MS: mass calcd. For C₈H₁₁N₃O: 165.09, found: 166.00 [M+H]⁺.

### Step 3: Synthesis of 4-({2-[4-(2,3-dimethoxybenzoyl)piperazin-1-yl]-2-oxoethyl}amino)-2-methylphenylurea (INT105-485-3)

To the mixture of 4-amino-2-methylphenylurea (1.00 g, 6.05 mmol, 1.00 equiv) and 2-bromo-1-[4-(2,3-dimethoxybenzoyl)piperazin-1-yl]ethanone (2.36 g, 6.36 mmol, 1.05 equiv) in DMF (20.00 mL) was added NaHCO₃ (1.02 g, 12.11 mmol, 2 equiv) and NaI (1.00 g, 6.66 mmol, 1.1 equiv). The mixture was stirred at 60° C. for 16.0 h. To the mixture was added H₂O (20 mL) and extracted by DCM (20 mL×3). The organic layer was combined, washed by H₂O (50 mL) and brine (50 mL), dried over Na₂SO₄, filtered and concentrated under reduced pressure. The residue was purified by reverse flash chromatography with the following conditions: column, C18 silica gel; mobile phase, MeOH in water, 10% to 50% gradient in 20 min; detector, UV 254 n. 740.00 mg of desired product was obtained as a black solid (26.84% yield). LC/MS: mass calcd. For C₂₃H₂₉N₅O₅: 455.22, found: 456.15 [M+H]⁺.

### Step 4: Synthesis of tert-butyl N-(1-{[4-(carbamoylamino)-3-methylphenyl] ({2-[4-(2,3-dimethoxybenzoyl)piperazin-1-yl]-2-oxoethyl})carbamoyl}-2,5,8,11,14-pentaoxahexadecan-16-yl)carbamate (INT105-485-4)

The procedure was the same as (Comp. 001). But the reaction was purified by reverse phase column. 100.00 mg of 4-({2-[4-(2,3-dimethoxybenzoyl)-1-yl]-2-oxoethyl}amino)-2-methylphenylurea was used, 60.00 mg of desired product was obtained as colorless oil (32.81% yield). LC/MS: mass calcd. For C₄₀H₆₀N₆O₁₃: 832.42, found: 855.40 [M+Na]⁺.

### Step 5: Synthesis of 17-amino-N-[4-(carbamoylamino)-3-methylphenyl]-N-{2-[4-(2,3-dimethoxybenzoyl)piperazin-1-yl]-2-oxoethyl}-3,6,9,12,15-pentaoxaheptadecanamide (INT105-485-5)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51). But the reaction time was 1.0 h. 60.00 mg of tert-butyl N-(1-{[4-(carbamoylamino)-3-methylphenyl]({2-[4-(2,3-dimethoxybenzoyl)piperazin-1-yl]-2-oxoethyl})carbamoyl}-2,5,8,11,14-pentaoxahexadecan-16-yl)carbamate was used, 40.00 mg of desired product was obtained as colorless oil (75.77% yield). LC/MS: mass calcd. For C₃₅H₅₂N₆O₁₁: 732.37, found: 733.35 [M+H]⁺.

### Step 6: Synthesis of N-{5-[(3-{[2-({2-[(5-{[2-({2-[(1-{[4-(carbamoylamino)-3-methylphenyl]({2-[4-(2,3-dimethoxybenzoyl)piperazin-1-yl]-2-oxoethyl})carbamoyl}-2,5,8,11,14-pentaoxahexadecan-16-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 143)

The procedure was the same as (INT61-04-OH-21), but the reaction mixture was purified by Prep-HPLC. 35.00 mg of 17-amino-N-[4-(carbamoylamino)-3-methylphenyl]-N-{2-[4-(2,3-dimethoxybenzoyl)piperazin-1-yl]-2-oxoethyl}-3,6,9,12,15-pentaoxaheptadecanamide was used, 4.60 mg of desired product was obtained as a white solid (4.62% yield). HRMS: mass calcd. For C₈₆H₁₁₁N₂₇O₂₂: 1873.8379, found: 1874.8405 [M+H]⁺.

### Example 43. Synthesis of Compound 146

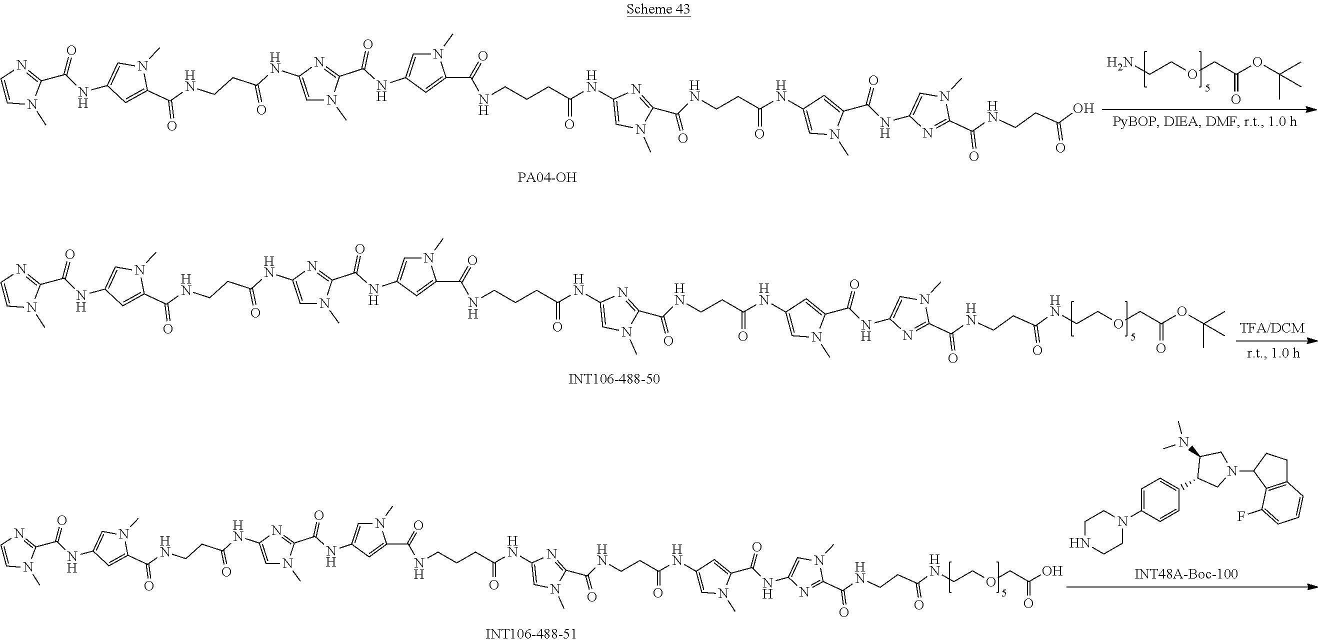

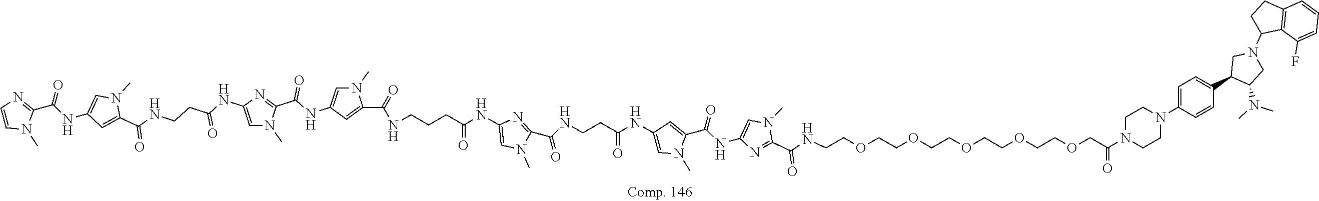 [SMILES: CN(C)[C@H]1CN(C2CCc3cccc(F)c32)C[C@@H]1c1ccc(N2CCN(C(=O)COCCOCCOCCOCCOCCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)CC2)cc1]

### Step 1: Synthesis of tert-butyl 17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecanoate (INT106-488-50)

The procedure was the same as (INT61-04-OH-21). 250.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 260.00 mg of desired product was obtained as yellow solid (80.78% yield). LC/MS: mass calcd. For C₆₇H₉₂N₂₂O₁₈:1492.70, found: 747.56[M/2+H]⁺.

### Step 2: Synthesis of 17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecanoic acid (INT106-488-51)

A solution of tert-butyl 17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecanoate (260.00 mg, 0.17 mmol, 1.00 equiv) and TFA (1 mL) in DCM (5 mL) was stirred for 1 h at room temperature. The resulting mixture was concentrated under vacuum. This resulted in 17-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxaheptadecanoic acid (300 mg, crude) as a yellow oil. LC/MS: mass calcd. For C₆₃H₈₄N₂₂O₁₈: 1436.63, found: 1437.80[M+H]⁺.

### Step 3: Synthesis of N-(5-{[3-({2-[(2-{[5-({2-[(2-{[17-(4-{4-[(3S,4R)-4-(dimethylamino)-1-(7-fluoro-2,3-dihydro-1H-inden-1-yl)pyrrolidin-3-yl]phenyl}piperazin-1-yl)-17-oxo-3,6,9,12,15-pentaoxaheptadecan-1-yl]carbamoyl}ethyl)carbamoyl]-methylimidazol-4-yl}carbamoyl)-1-methylpyrrol-3-yl]carbamoyl}ethyl)carbamoyl]-1-methylimidazol-4-yl}carbamoyl)propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Comp. 146)

The procedure was the same as (INT61-04-OH-21), but the reaction mixture was purified by Prep-HPLC. 25.00 mg of (3R,4S)-1-(7-fluoro-2,3-dihydro-1H-inden-1-yl)-N,N-dimethyl-4-[4-(piperazin-1-yl)phenyl]pyrrolidin-3-amine was used, 11.40 mg of desired product was obtained s white solid (9.53% yield). HRMS: mass calcd. For C₈₈H₁₁₅FN₂₆O₁₇: 1826.8918, found: 1827.8891[M+H]⁺.

### Example 44. Synthesis of Synthesis of Compound 147

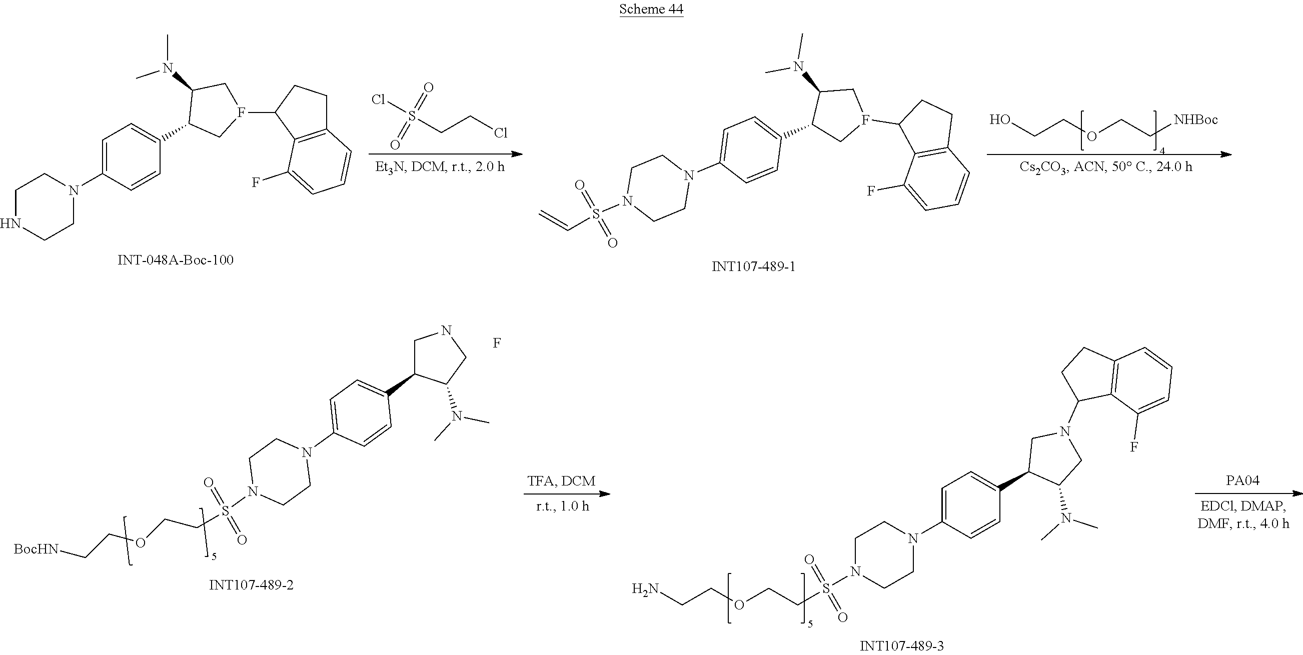

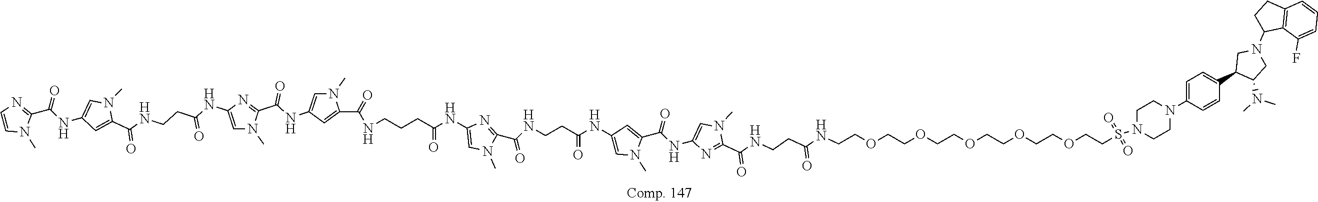 [SMILES: CN(C)[C@H]1CN(C2CCc3cccc(F)c32)C[C@@H]1c1ccc(N2CCN(S(=O)(=O)CCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)CC2)cc1]

### Step 1: Synthesis of (3R,4S)-4-{4-[4-(ethenesulfonyl)piperazin-1-yl]phenyl}-1-(7-fluoro-2,3-dihydro-1H-inden-1-yl)-N,N-dimethylpyrrolidin-3-amine (INT107-489-1)

A solution of (3R,4S)-1-(7-fluoro-2,3-dihydro-1H-inden-1-yl)-N,N-dimethyl-4-[4-(piperazin-1-yl)phenyl]pyrrolidin-3-amine (200.00 mg, 0.49 mmol, 1.00 equiv) and 2-chloroethanesulfonyl chloride (190.00 mg, 1.17 mmol, 2.38 equiv), TEA (158.00 mg, 1.56 mmol, 3.19 equiv) in DCM (5.00 mL) was stirred for 2.0 h at room temperature. The resulting mixture was concentrated under vacuum. The residue was purified by silica gel column chromatography, eluted with CH₂Cl₂/MeOH (10:1) to afford (3R,4S)-4-{4-[4-(ethenesulfonyl)piperazin-1-yl]phenyl}-1-(7-fluoro-2,3-dihydro-1H-inden-1-yl)-N,N-dimethylpyrrolidin-3-amine (70.00 mg, 28.68% yield) as a white solid. LC/MS: mass calcd. For C₂₇H₃₅FN₄O₂S: 498.25, found: 499.20[M+H]⁺.

### Step 2: Synthesis of tert-butyl N-[17-(4-{4-[(3S,4R)-4-(dimethylamino)-1-(7-fluoro-2,3-dihydro-1H-inden-1-yl)pyrrolidin-3-yl]phenyl}piperazin-1-ylsulfonyl)-3,6,9,12,15-pentaoxaheptadecan-1-yl]carbamate (INT107-489-2)

A mixture of (3R,4S)-4-{4-[4-(ethenesulfonyl)piperazin-1-yl]phenyl}-1-(7-fluoro-2,3-dihydro-1H-inden-1-yl)-N,N-dimethylpyrrolidin-3-amine (60.00 mg, 0.12 mmol, 1.00 equiv) and tert-butyl N-(14-hydroxy-3,6,9,12-tetraoxatetradecan-1-yl)carbamate (41.00 mg, 0.12 mmol, 1.01 equiv), Cs₂CO₃ (117.00 mg, 0.36 mmol, 2.98 equiv) in ACN (3.00 mL) was stirred for 24.0 h at 50° C. The mixture was allowed to cool down to room temperature. The resulting mixture was filtered and the filter cake was washed with ACN (3×3 mL). The filtrate was concentrated under reduced pressure. The residue was purified by Prep-TLC (CH₂Cl₂/MeOH 10:1) to afford tert-butyl N-[17-(4-{4-[(3S,4R)-4-(dimethylamino)-1-(7-fluoro-2,3-dihydro-1H-inden-1-yl)pyrrolidin-3-yl]phenyl}piperazin-1-ylsulfonyl)-3,6,9,12,15-pentaoxaheptadecan-1-yl]carbamate (65.00 mg, 64.61%) as a white oil. LC/MS: mass calcd. For C₄₂H₆₆FN₅O₉S: 835.46, found: 858.40[M+Na]⁺.

### Step 3: Synthesis of (3R, 4S)-4-{4-[4-(17-amino-3,6,9,12,15-pentaoxaheptadecane-1-sulfonyl)piperazin-1-yl]phenyl}-1-(7-fluoro-2,3-dihydro-1H-inden-1-yl)-N,N-dimethylpyrrolidin-3-amine (INT107-489-3)

The procedure was the same as (INT-29-110). 30.00 mg of tert-butyl N-[17-(4-{4-[(3S,4R)-4-(dimethylamino)-1-(7-fluoro-2,3-dihydro-1H-inden-1-yl)pyrrolidin-3-yl]phenyl}piperazin-1-ylsulfonyl)-3,6,9,12,15-pentaoxaheptadecan-1-yl]carbamate was used, 30.00 mg crude of desired product was obtained as yellow oil. LCMS: mass calcd. For C₃₇H₅₈FN₅O₇S: 735.40, found: 758.70[M+Na]⁺.

### Step 4: Synthesis of N-(5-{[3-({2-[(2-{[5-({2-[(2-{[17-(4-{4-[(3S,4R)-4-(dimethylamino)-1-(7-fluoro-2,3-dihydro-1H-inden-1-yl)pyrrolidin-3-yl]phenyl}piperazin-1-ylsulfonyl)-3,6,9,12,15-pentaoxaheptadecan-1-yl]carbamoyl}ethyl)carbamoyl]-1-methylimidazol-4-yl}carbamoyl)-1-methylpyrrol-3-yl]carbamoyl}ethyl)carbamoyl]-1-methylimidazol-4-yl}carbamoyl)propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Comp. 147)

The procedure was the same as (INT61-025-20), but the reaction temperature was r.t., the reaction time was 4.0 h. 45.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 11.10 mg of desired product was obtained as white solid (14.36% yield). HRMS: mass calcd. For C₈₈H₁₁₇FN₂₆O₁₈S: 1876.8744, found: 1877.8772[M+H]⁺.

### Example 45. Synthesis of Compound 159

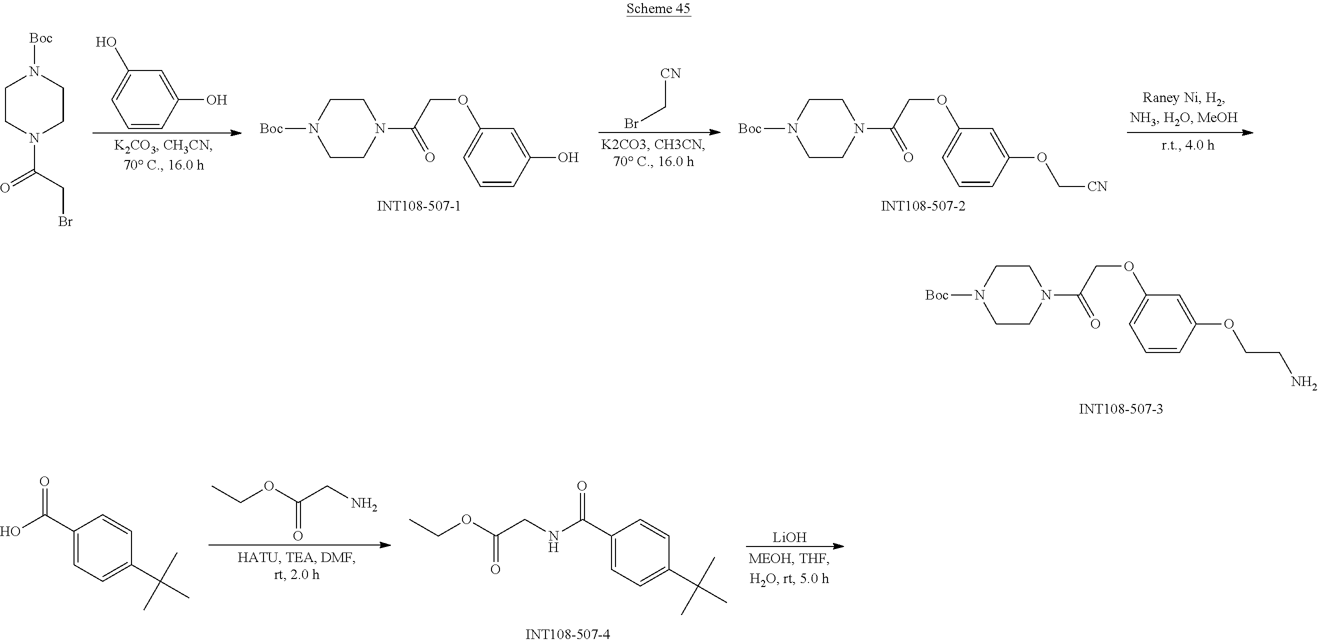

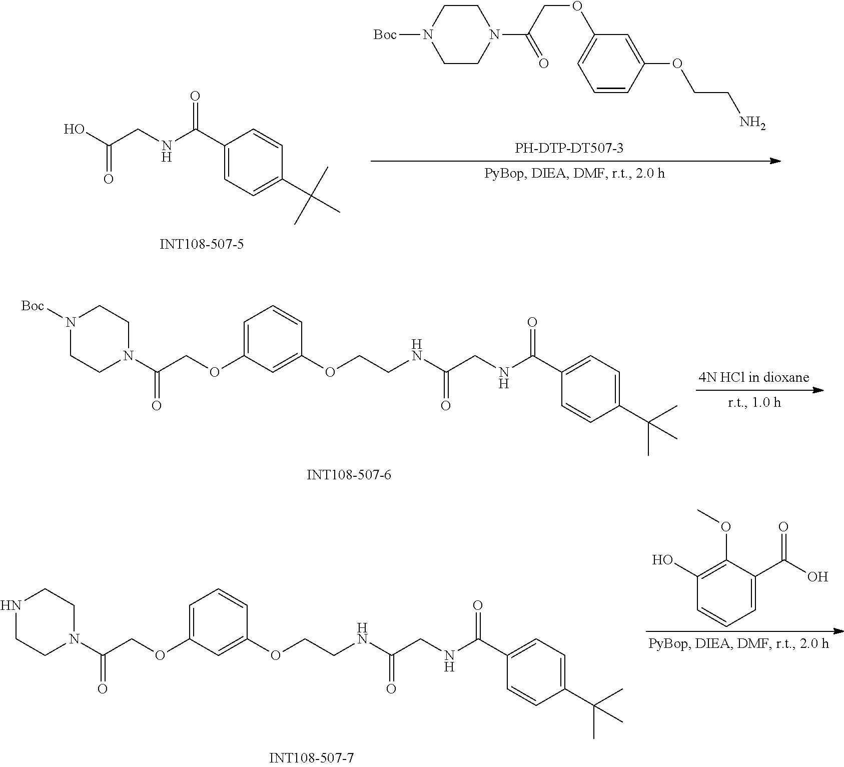 [SMILES: CC(C)(C)c1ccc(C(=O)NCC(=O)O)cc1 ; CC(C)(C)OC(=O)N1CCN(C(=O)COc2cccc(OCCN)c2)CC1 ; CC(C)(C)OC(=O)N1CCN(C(=O)COc2cccc(OCCNC(=O)CNC(=O)c3ccc(C(C)(C)C)cc3)c2)CC1 ; CC(C)(C)c1ccc(C(=O)NCC(=O)NCCOc2cccc(OCC(=O)N3CCNCC3)c2)cc1 ; COc1c(O)cccc1C(=O)O]

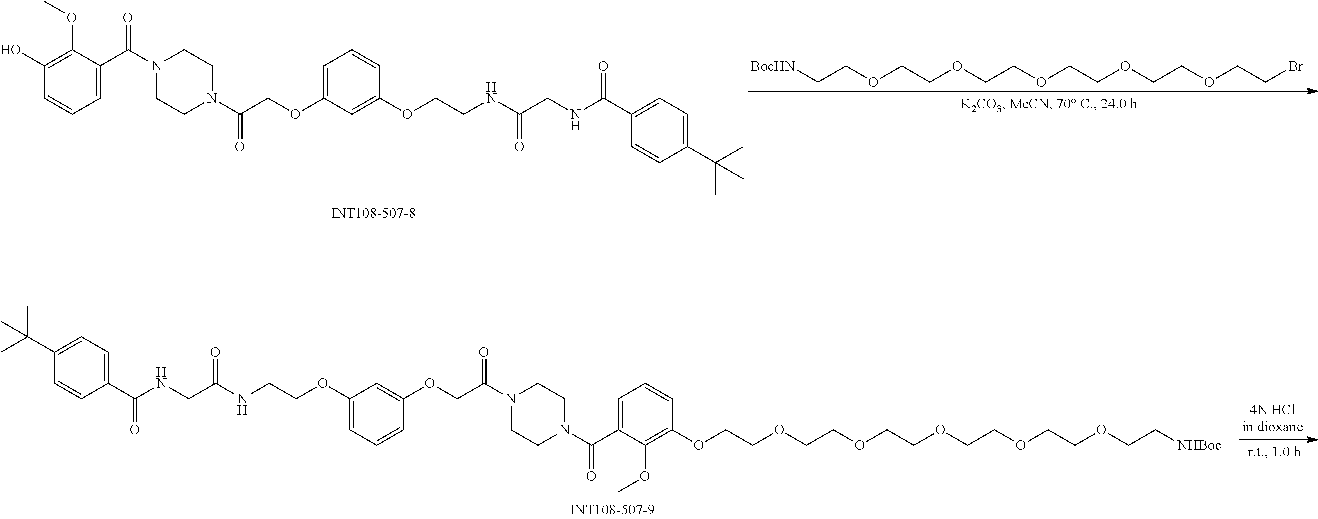 [SMILES: COc1c(O)cccc1C(=O)N1CCN(C(=O)COc2cccc(OCCNC(=O)CNC(=O)c3ccc(C(C)(C)C)cc3)c2)CC1 ; CC(C)(C)OC(=O)NCCOCCOCCOCCOCCOCCBr ; COc1c(OCCOCCOCCOCCOCCOCCNC(=O)OC(C)(C)C)cccc1C(=O)N1CCN(C(=O)COc2cccc(OCCNC(=O)CNC(=O)c3ccc(C(C)(C)C)cc3)c2)CC1]

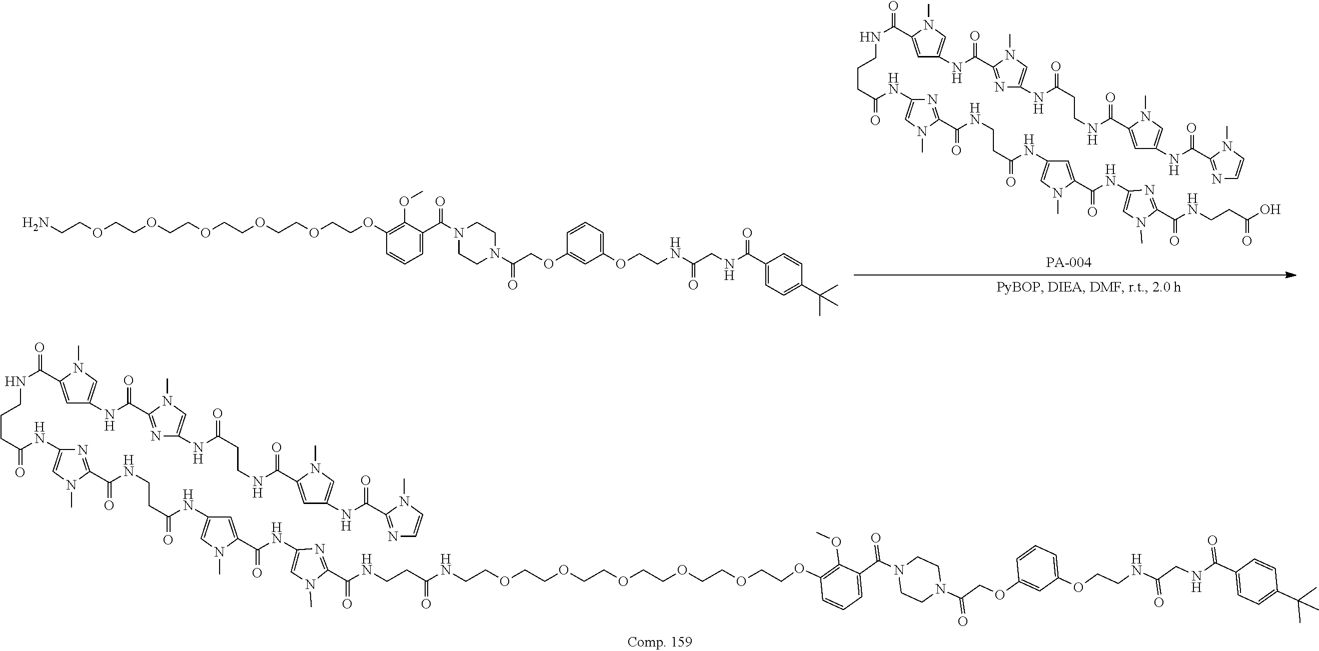 [SMILES: COc1c(OCCOCCOCCOCCOCCOCCN)cccc1C(=O)N1CCN(C(=O)COc2cccc(OCCNC(=O)CNC(=O)c3ccc(C(C)(C)C)cc3)c2)CC1 ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1 ; COc1c(OCCOCCOCCOCCOCCOCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cccc1C(=O)N1CCN(C(=O)COc2cccc(OCCNC(=O)CNC(=O)c3ccc(C(C)(C)C)cc3)c2)CC1]

### Step 1: Synthesis of tert-butyl 4-[2-(3-hydroxyphenoxy)acetyl]piperazine-1-carboxylate (INT108-507-1)

The procedure was the same as (INT102-388-1). But the solvent was MeCN. 2.00 g of tert-butyl 4-(2-bromoacetyl)piperazine-1-carboxylate was used, 1.20 g of desired product was obtained as a white solid (54.79% yield). LC/MS: mass calcd. For C₁₇H₂₄N₂O₅: 336.17, found: 281.10 [M+H−tBu]⁺.

### Step 2: Synthesis of tert-butyl 4-{2-[3-(cyanomethoxy)phenoxy]acetyl}piperazine-1-carboxylate (INT108-507-2)

The procedure was the same as (INT102-388-1). But the solvent was MeCN. 1.20 g of tert-butyl 4-[2-(3-hydroxyphenoxy)acetyl]piperazine-1-carboxylate was used, 1.10 g of desired product was obtained as a yellow solid (82.14% yield.) LC/MS: mass calcd. For C₁₉H₂₅N₃O₅: 375.18, found: 320.05 [M+H−tBu]⁺.

### Step 3: Synthesis of tert-butyl 4-{2-[3-(2-aminoethoxy)phenoxy]acetyl}piperazine-1-carboxylate (INT108-507-3)

To the mixture of tert-butyl 4-{2-[3-(cyanomethoxy)phenoxy]acetyl}piperazine-1-carboxylate (1.00 g, 2.66 mmol, 1.00 equiv) in NH3H2O (10.00 mL, 256.81 mmol, 96.41 equiv) and MeOH (15.00 mL, 370.48 mmol, 139.09 equiv) was added Raney Ni (22.82 g, 26.64 mmol, 10.00 equiv). The mixture was stirred at r.t. for 4.0 h under H₂ atmosphere. The resulting mixture was filtered, the filter cake was washed with MeOH. The filtrate was concentrated under reduced pressure. The residue was purified by reverse flash chromatography with the following conditions: column, C18 silica gel; mobile phase, MeOH in water (0.05% TFA), 10% to 50% gradient in 20 min; detector, UV 254 nm. 250.00 mg of desired product was obtained as an off-white solid (24.73% yield). LC/MS: mass calcd. For C₁₉H₂₉N₃O₅: 379.21, found: 323.10 [M+H-tBu]⁺.

### Step 4: Synthesis of ethyl 2-[(4-tert-butylphenyl)formamido]acetate (INT108-507-4)

To the mixture of 4-tert-butylbenzoic acid (2.00 g, 11.22 mmol, 1.00 equiv) and amino-acetic acid ethyl ester (1.16 g, 11.22 mmol, 1.00 equiv) in DMF (20.00 mL, 258.44 mmol, 23.03 equiv) was added HATU (6.40 g, 16.83 mmol, 1.50 equiv) and TEA (2.84 g, 28.05 mmol, 2.50 equiv). The mixture was stirred at r.t. for 2.0 h. To the mixture was added H2O (30 mL) and extracted by EtAOc (30 mL×3). The organic layer was combined, washed by H2O (50 mL) and brine (50 mL×2), dried over Na₂SO₄, filtered and concentrated under reduced pressure. The residue was purified by silica gel column chromatography, eluted with PE/EA (1:1). 2.50 g of desired product was obtained as a white solid (84.60% yield). LC/MS: mass calcd. For C₁₅H₂₁NO₃: 263.15, found: 264.15 [M+H]⁺.

### Step 5: Synthesis of [(4-tert-butylphenyl)formamido]acetic acid (INT108-507-5)

The procedure was the same as 4-[3-[(Tert-butoxycarbonyl)amino] propanamido]-1-methylimidazole-2-carboxylic acid (INT60-022-2000). 2.40 g of 2-[(4-tert-butylphenyl)formamido]acetate was used, 2.00 g of desired product was obtained as a white solid (93.27% yield). LC/MS: mass calcd. For C₁₃H₁₇NO₃: 235.12, found: 236.15 [M+H]⁺.

### Step 6: Synthesis of tert-butyl 4-{2-[3-(2-{2-[(4-tert-butylphenyl)formamido]acetamido}ethoxy)phenoxy]acetyl}piperazine-1-carboxylate (INT108-507-6)

The procedure was the same as (INT61-04-OH-21). But the reaction time was 2.0 h. 160.00 mg of [(4-tert-butylphenyl)formamido]acetic acid was used, 130.00 mg of desired product was obtained as colorless oil (32.04% yield). LC/MS: mass calcd. For C₃₂H₄₄N₄O₇: 596.32, found: 619.30 [M+Na]⁺.

### Step 7: Synthesis of 2-[(4-tert-butylphenyl)formamido]-N-(2-{3-[2-oxo-2-(piperazin-1-yl)ethoxy]phenoxy}ethyl)acetamide (INT108-507-7)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51). 130.00 mg of tert-butyl 4-{2-[3-(2-{2-[(4-tert-butylphenyl)formamido]acetamido}ethoxy)phenoxy]acetyl}piperazine-1-carboxylate was used, 100.00 mg of desired product was obtained as colorless oil (92.43% yield). LC/MS: mass calcd. For C₂₇H₃₆N₄O₅: 496.27, found: 497.25 [M+H]⁺.

### Step 8: Synthesis of 2-[(4-tert-butylphenyl)formamido]-N-[2-(3-{2-[4-(3-hydroxy-2-methoxybenzoyl)piperazin-1-yl]-2-oxoethoxy}phenoxy)ethyl]acetamide (INT108-507-80)

The procedure was the same as (INT61-04-OH-21). But the reaction time was 2.0 h. 130.00 mg of 2-[(4-tert-butylphenyl)formamido]-N-(2-{3-[2-oxo-2-(piperazin-1-yl)ethoxy]phenoxy}ethyl)acetamide was used. 90.00 mg of desired product was obtained as colorless oil (53.16% yield). LC/MS: mass calcd. For C₃₅H₄₂N₄O₈:646.30, found: 647.20 [M+H]⁺.

### Step 9: Synthesis of tert-butyl N-{17-[3-(4-{2-[3-(2-{2-[(4-tert-butylphenyl)formamido]acetamido}ethoxy)phenoxy]acetyl}piperazine-1-carbonyl)-2-methoxyphenoxy]-3,6,9,12,15-pentaoxaheptadecan-1-yl}carbamate (INT108-507-9)

The procedure was the same as (INT102-388-1). But the reaction time was 2.0 h and the solvent was MeCN. 80.00 mg of 2-[(4-tert-butylphenyl)formamido]-N-[2-(3-{2-[4-(3-hydroxy-2-methoxybenzoyl)piperazin-1-yl]-2-oxoethoxy}phenoxy)ethyl]acetamide was used, 60.00 mg of desired product was obtained as colorless oil (48.02% yield). LC/MS: mass calcd. For C₅₂H₇₅N₅O₁₅:1009.53, found: 1010.50 [M+H]⁺.

### Step 10: Synthesis of N-(2-{3-[2-(4-{3-[(17-amino-3,6,9,12,15-pentaoxaheptadecan-1-yl)oxy]-2-methoxybenzoyl}piperazin-1-yl)-2-oxoethoxy]phenoxy}ethyl)-2-[(4-tert-butylphenyl)formamido]acetamide (INT108-507-10)

The procedure was the same as (INT91-010-51). But the reaction time was 1.0 h. 50.00 mg of tert-butyl N-{17-[3-(4-{2-[3-(2-{2-[(4-tert-butylphenyl) formamido]acetamido}ethoxy)phenoxy]acetyl}piperazine-1-carbonyl)-2-methoxyphenoxy]-3,6,9,12,15-pentaoxaheptadecan-1-yl}carbamate was used, 40.00 mg of desired product was obtained as colorless oil (88.80% yield). LC/MS: mass calcd. For C₄₇H₆₇N₅O₁₃: 909.47, found: 910.60 [M+H]⁺.

### Step 11: Synthesis of N-[5-({3-[(2-{[2-({5-[(2-{[2-([17-[3-(4-{2-[3-(2-{2-[(4-tert- butylphenyl)formamido]acetamido}ethoxy)phenoxy]acetyl}piperazine-1-carbonyl)-2-methoxyphenoxy]-3,6,9,12,15-pentaoxaheptadecan-1-yl}carbamoyl)ethyl]carbamoyl}-1-methylimidazol-4-yl)carbamoyl]-1-methylpyrrol-3-yl}carbamoyl)ethyl]carbamoyl}-1-methylimidazol-4-yl)carbamoyl]propyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide (Comp. 159)

The procedure was the same as (INT61-04-OH-21), but the reaction time was 2.0 h and the reaction mixture was purified by Prep-HPLC. 30.00 mg of N-(2-{3-[2-(4-{3-[(17-amino-3,6,9,12,15-pentaoxaheptadecan-1-yl)oxy]-2-methoxybenzoyl}piperazin-1-yl)-2-oxoethoxy]phenoxy}ethyl)-2-[(4-tert-butylphenyl)formamido]acetamide was used, 6.60 mg of desired product was obtained as a white solid (9.51% yield). HRMS: mass calcd. For C₉₈H₁₂₆N₂₆O₂₄: 2050.9438, found: 2051.9442 [M+H]⁺.

### Example 46. Synthesis of Compound 163

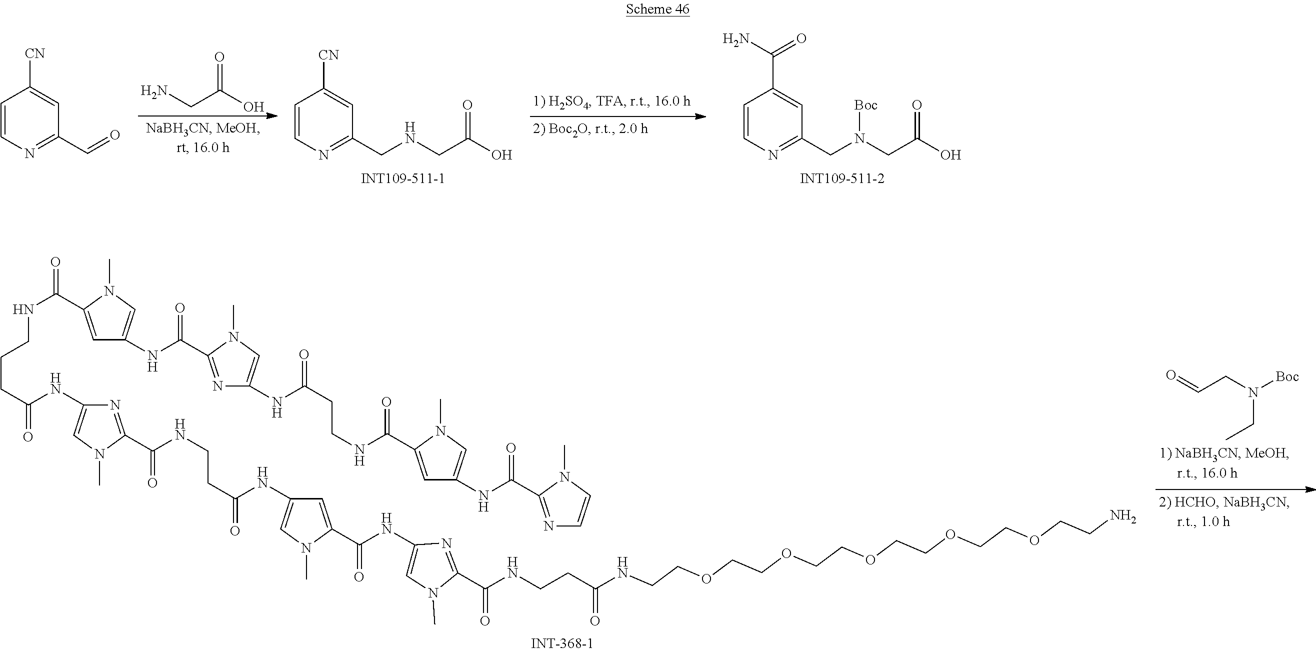 [SMILES: N#Cc1ccnc(C=O)c1 ; NCC(=O)O ; N#Cc1ccnc(CNCC(=O)O)c1 ; CC(C)(C)OC(=O)N(CC(=O)O)Cc1cc(C(N)=O)ccn1 ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOCCN)n3)n(C)c2)n1 ; CCN(CC=O)C(=O)OC(C)(C)C]

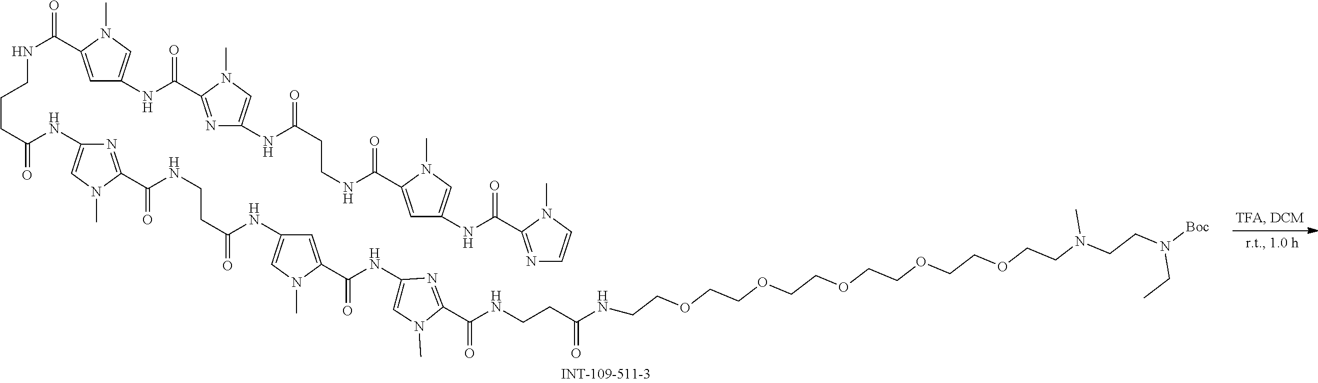 [SMILES: CCN(CCN(C)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)C(=O)OC(C)(C)C]

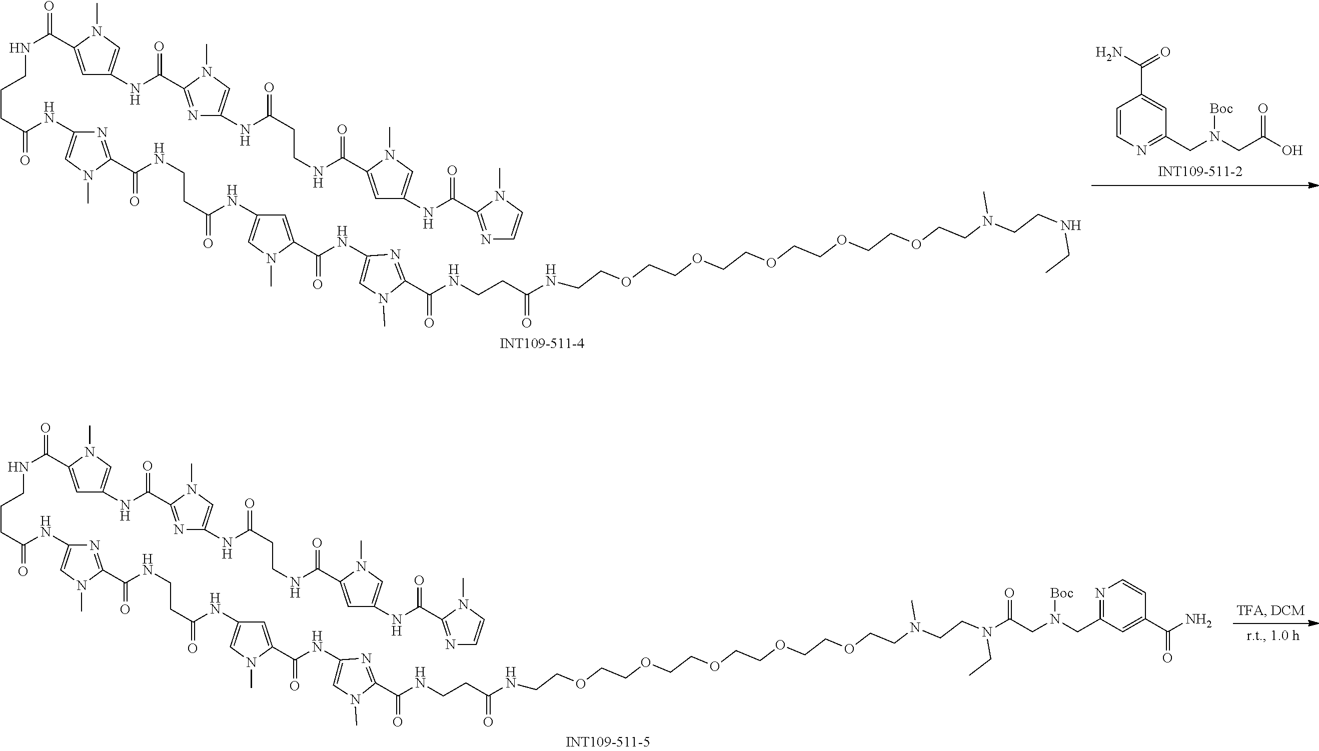 [SMILES: CCNCCN(C)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C ; CC(C)(C)OC(=O)N(CC(=O)O)Cc1cc(C(N)=O)ccn1 ; CCN(CCN(C)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)C(=O)CN(Cc1cc(C(N)=O)ccn1)C(=O)OC(C)(C)C]

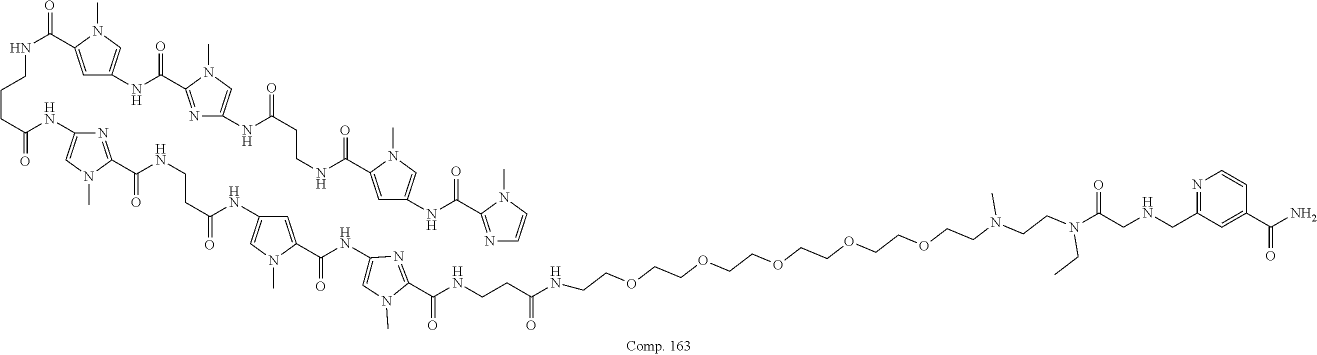 [SMILES: CCN(CCN(C)CCOCCOCCOCCOCCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)C(=O)CNCc1cc(C(N)=O)ccn1]

### Step 1: Synthesis of {[(4-cyanopyridin-2-yl)methyl]amino}acetic acid (INT109-511-1)

To a stirred mixture of 2-formylpyridine-4-carbonitrile (300.00 mg, 2.27 mmol, 1.00 equiv) and glycine (170.45 mg, 2.27 mmol, 1.00 equiv) in MeOH (5.00 mL) was added NaBH₃CN (285.38 mg, 4.54 mmol, 2.00 equiv) at room temperature. The reaction was stirred 16.0 h. The reaction mixture was filtered through a Celite pad. The filtrate was concentrated under reduced pressure. The residue was purified by reverse flash chromatography with the following conditions: column, silica gel; mobile phase, MeCN in water (0.05% TFA), 1% to 10% gradient in 10 min; detector, UV 254 nm to afford {[(4-cyanopyridin-2-yl)methyl]amino}acetic acid (310.00 mg, 71.41% yield) as light yellow oil. LC/MS: mass calcd. For C₉H₉N₃O₂: 191.07, found: 192.10 [M+H]⁺.

### Step 2: Synthesis of ((4-carbamoylpyridin-2-yl)methyl)glycine (INT109-511-2)

A mixture of {[(4-cyanopyridin-2-yl)methyl]amino}acetic acid (310.00 mg, 1.62 mmol, 1.00 equiv) and H₂SO₄ (1.00 mL, 18.76 mmol, 11.57 equiv), TFA (4.00 mL, 53.85 mmol, 33.21 equiv) was stirred overnight at room temperature. Desired product could be detected by LCMS. The mixture was basified to pH=10 with NaHCO₃. LC/MS: mass calcd. For C₉H₁₁N₃O₃: 209.08, found: 210.10 [M+H]⁺. The mixture was added with di-tert-butyl dicarbonate (1.00 mL). The reaction was stirred for 2.0 h at room temperature. The reaction worked according to LCMS. The mixture was concentrated under reduced pressure. The residue was purified by reverse flash chromatography with the following conditions: column, silica gel; mobile phase, MeCN in water, 10% to 60% gradient in 20 min; detector, UV 254 nm to afford [(tert-butoxycarbonyl)[(4-carbamoylpyridin-2-yl)methyl]amino]acetic acid (60.00 mg, 11.96% yield) as light yellow oil. LC/MS: mass calcd. For C₁₄H₁₉N₃O₅: 309.13, found: 310.30 [M+H]⁺.

### Step 3: Synthesis of yl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxa-18-azaicosan-20-yl)carbamate (INT-109-511-3)

To a stirred mixture of N-{5-[(3-{[2-({2-[(5-{[2-({2-[(17-amino-3,6,9,12,15-pentaoxaheptadecan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (140.00 mg, 0.10 mmol, 1.00 equiv) and tert-butyl N-ethyl-N-(2-oxoethyl)carbamate (36.85 mg, 0.20 mmol, 2.00 equiv) in MeOH (5.00 mL) was added NaBH₃CN (24.74 mg, 0.39 mmol, 4.00 equiv) at room temperature. The reaction was stirred overnight.

The reaction mixture was used in the next step directly without further purification. LC/MS: mass calcd. For C₇₂H₁₀₄N₂₄O₁₈: 1592.80, found: 797.80 [1/2M+H]⁺. The resulting mixture were added with formaldehyde (50.00 mg, 1.67 mmol, 14.74 equiv) and NaBH₃CN (14.19 mg, 0.23 mmol, 2.00 equiv) at room temperature. The reaction was stirred for 1.0 h. Desired product could be detected by LCMS. The reaction mixture was concentrated under reduced pressure. The residue was purified by reverse flash chromatography with the following conditions: column, silica gel; mobile phase, MeCN in water (0.05% TFA), 10% to 50% gradient in 20 min; detector, UV 254 nm to afford tert-butyl N-ethyl-N-(18-methyl-1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxa-18-azaicosan-20-yl)carbamate (180.00 mg, 99.13% yield) as colorless oil. LC/MS: mass calcd. For C₇₃H₁₀₆N₂₄O₁₈: 1606.81, found: 804.75 [1/2M+H]⁺.

### Step 4: Synthesis of 1-methyl-4-(3-{[1-methyl-4-(1-methylimidazole-2-amido)pyrrol-2-yl]formamido}propanamido)-N-{1-methyl-5-[(3-{[1-methyl-2-({2-[(1-methyl-5-{[1-methyl-2-({2-[(18-methyl-3,6,9,12,15-pentaoxa-18,21-diazatricosan-1-yl)carbamoyl]ethyl}carbamoyl)imidazol-4-yl]carbamoyl}pyrrol-3-yl)carbamoyl]ethyl}carbamoyl)imidazol-4-yl]carbamoyl}propyl)carbamoyl]pyrrol-3-yl}imidazole-2-carboxamide (INT109-511-4)

The procedure was the same as (INT-29-110). After the reaction, the reaction mixture was purified by Prep-HPLC. 180.00 mg of tert-butyl N-ethyl-N-(18-methyl-1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxa-18-azaicosan-20-yl)carbamate was used. 20.00 mg of desired product was obtained as an off-white solid (11.85% yield). LC/MS: mass calcd. For C₆₈H₉₈N₂₄O₁₆: 1506.76, found: 754.65 [1/2M+H]⁺.

### Step 5: Synthesis of tert-butyl N-[(4-carbamoylpyridin-2-yl)methyl]-N-{[ethyl(18-methyl-1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,1S-pentaoxa-18-azaicosan-20-yl)carbamoyl]methyl}carbamate (INT109-511-5)

The procedure was the same as (INT61-04-OH-21), but the reaction time was 2.0 h. 40.00 mg of 1-methyl-4-(3-{[1-methyl-4-(1-methylimidazole-2-amido)pyrrol-2-yl]formamido}propanamido)-N-{1-methyl-5-[(3-{[1-methyl-2-({2-[(1-methyl-5-{[1-methyl-2-({2-[(18-methyl-3,6,9,12,15-pentaoxa-18,21-diazatricosan-1-yl)carbamoyl]ethyl}carbamoyl)imidazol-4-yl]carbamoyl}pyrrol-3-yl)carbamoyl]ethyl}carbamoyl)imidazol-4-yl]carbamoyl}propyl)carbamoyl]pyrrol-3-yl}imidazole-2-carboxamide was used. 20.00 mg of desired product was obtained as a light yellow solid (41.90% yield). LC/MS: mass calcd. For C₈₂H₁₁₅N₂₇O₂₀: 1797.88, found: 900.60 [M+H]⁺.

### Step 6: Synthesis of 2-[({[ethyl(18-methyl-1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxa-18-azaicosan-20-yl)carbamoyl]methyl}amino)methyl]pyridine-4-carboxamide (Comp. 163)

The procedure was the same as (INT-29-110). After the reaction, the reaction mixture was purified by Prep-HPLC. 20.00 mg of tert-butyl N-[(4-carbamoylpyridin-2-yl)methyl]-N-{[ethyl(18-methyl-1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}-3,6,9,12,15-pentaoxa-18-azaicosan-20-yl)carbamoyl]methyl}carbamate was used. 3.40 mg of desired product was obtained as brown oil (14.49% yield). HRMS: mass calcd. For C₇₇H₁₀₇N₂₇O₁₈: 1697.8287, found: 1698.8281 [M+H]⁺.

### Example 47. Synthesis of Compound 164

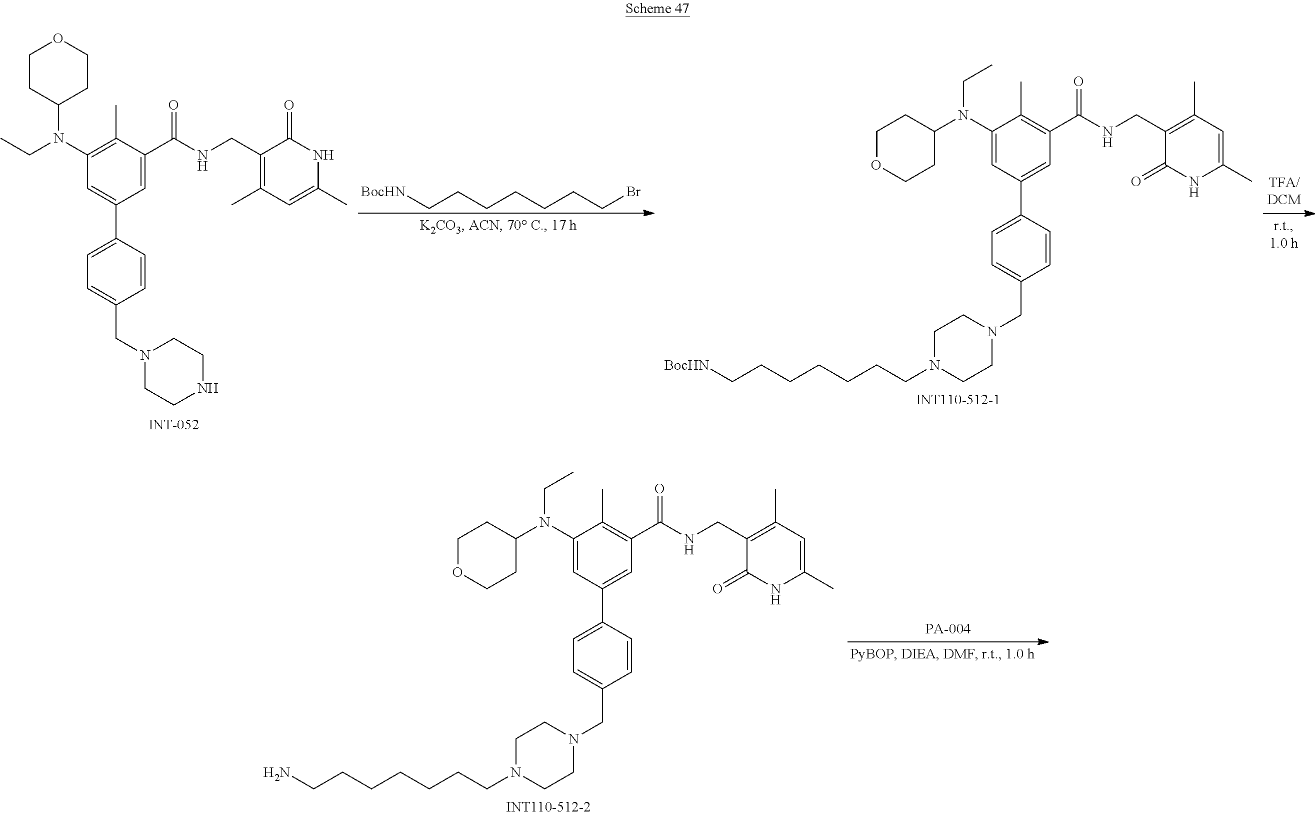 [SMILES: CCN(c1cc(-c2ccc(CN3CCNCC3)cc2)cc(C(=O)NCc2c(C)cc(C)[nH]c2=O)c1C)C1CCOCC1 ; CC(C)(C)OC(=O)NCCCCCCCBr ; CCN(c1cc(-c2ccc(CN3CCN(CCCCCCCNC(=O)OC(C)(C)C)CC3)cc2)cc(C(=O)NCc2c(C)cc(C)[nH]c2=O)c1C)C1CCOCC1 ; CCN(c1cc(-c2ccc(CN3CCN(CCCCCCCN)CC3)cc2)cc(C(=O)NCc2c(C)cc(C)[nH]c2=O)c1C)C1CCOCC1]

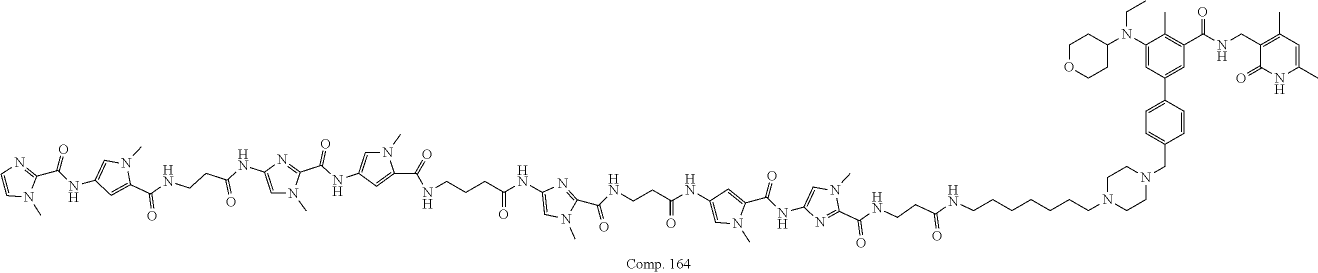 [SMILES: CCN(c1cc(-c2ccc(CN3CCN(CCCCCCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)cc2)cc(C(=O)NCc2c(C)cc(C)[nH]c2=O)c1C)C1CCOCC1]

### Step 1: Synthesis of tert-butyl N-(7-{4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazin-1-yl}heptyl)carbamate (INT110-512-1)

The procedure was the same as tert-butyl N-[2-(2-{2-[benzyl(methyl) amino]ethoxy}ethoxy)ethyl]carbamate (INT102-388-1). After the reaction, the reaction mixture was filtered and the filtrated was concentrated. The residue was purified by reverse phase column. 50.00 mg of N-[(4,6-2-oxo-1H-pyridin-3-yl)methyl]-5-[ethyl(oxan-4-yl)amino]-4-methyl-4′-(piperazin-1-ylmethyl)-[1,1′-biphenyl]-3-carboxamide was used, 28.90 mg of desired product was obtained as white solid (40.41% yield). LC/MS: mass calcd. For C₄₆H₆₈N₆O₅: 784.53, found: 785.80 [M+H]⁺.

### Step 2: Synthesis of 4′-{[4-(7-aminoheptyl)piperazin-1-yl] methyl}-N-[(4,6-dimethyl-2-oxo-1H-pyridin-3-yl)methyl]-5-[ethyl(oxan-4-yl)amino]-4-methyl-[1,1′-biphenyl]-3-carboxamide (INT110-512-2)

The procedure was the same as (INT-29-110). 23.00 mg of tert-butyl N-(7-{4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazin-1-yl}heptyl)carbamate was used, 22.00 mg crude of desired product was obtained as yellow oil. LC/MS: mass calcd. For C₄₁H₆₀N₆O₃: 684.47, found: 685.75 [M+H]⁺.

### Step 3: Synthesis of N-{5-[(3-{[2-({2-[(5-{[2-({2-[(7-{4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazin-1-yl}heptyl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 164)

The procedure was the same as (INT61-04-OH-21), but the reaction mixture was purified by Prep-HPLC. 33.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 10.70 mg of desired product was obtained as white solid (20.21% yield). HRMS: mass calcd. For C₉₂H₁₁₉N₂₇O₁₄: 1825.9430, found: 1826.9410[M+H]⁺.

### Example 48. Synthesis of Synthesis of Compound 167

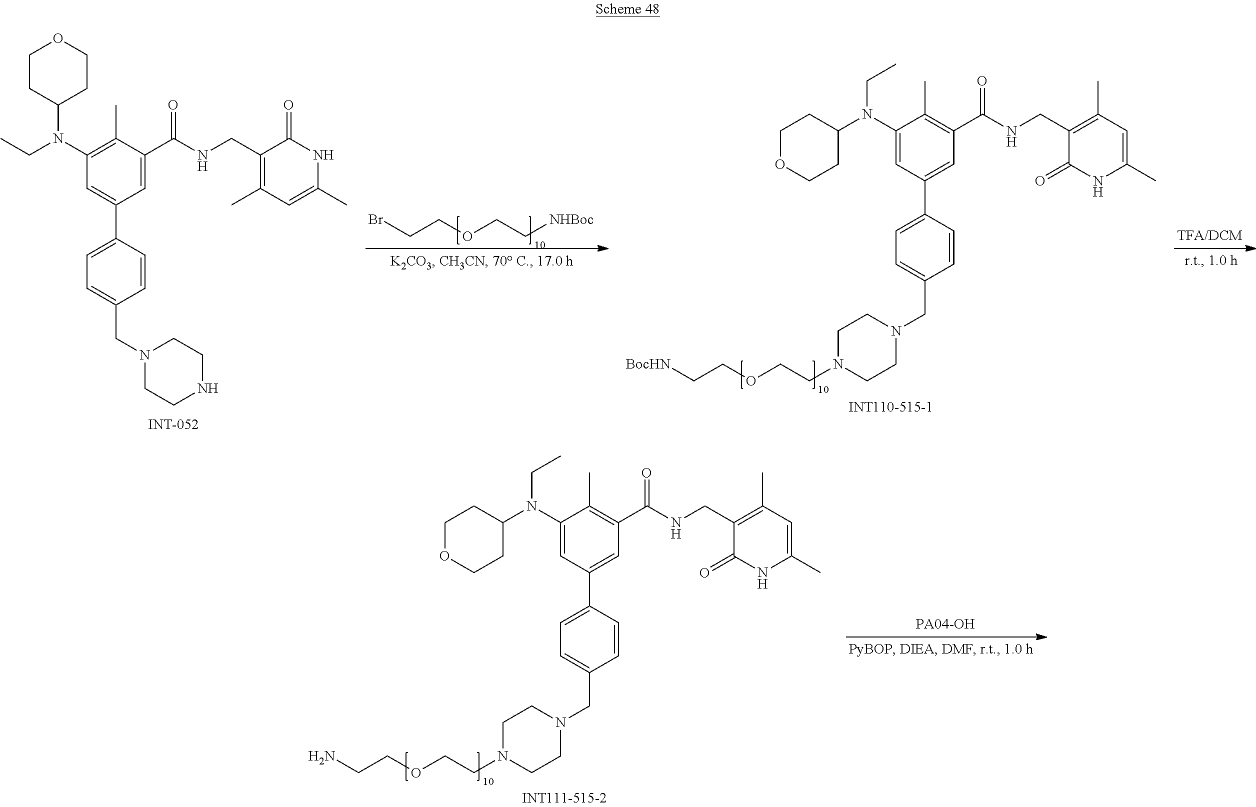

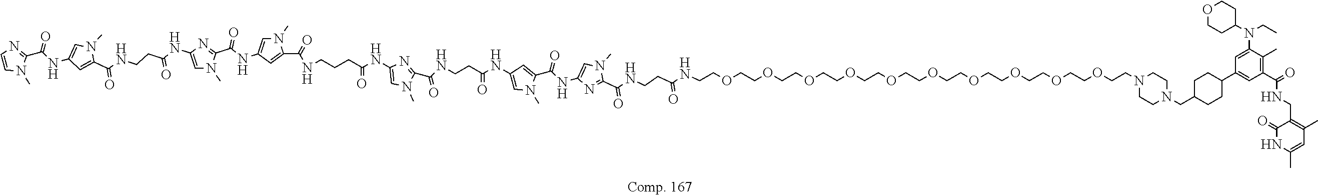 [SMILES: CCN(c1cc(C2CCC(CN3CCN(CCOCCOCCOCCOCCOCCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)CC2)cc(C(=O)NCc2c(C)cc(C)[nH]c2=O)c1C)C1CCOCC1]

### Step 1: Synthesis of tert-butyl N-(32-{4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazin-1-yl}-3,6,9,12,15,18,21,24,27,30-decaoxadotriacontan-1-yl)carbamate (INT111-515-1)

The procedure was the same as tert-butyl N-[2-(2-{2-[benzyl(methyl) amino]ethoxy}ethoxy)ethyl]carbamate (INT102-388-1), but the reaction mixture was purified by PLC-Plate. 30.00 mg of N-[(4,6-dimethyl-2-oxo-1H-pyridin-3-yl) methyl]-5-[ethyl(oxan-4-yl)amino]-4-methyl-4′-(piperazin-1-ylmethyl)-[1,1′-biphenyl]-3-carboxamide was used, 44.60 mg of desired product was obtained as white solid (73.56% yield). LC/MS: mass calcd. for C₆₁H₉₈N₆O₁₅: 1154.70, found: 578.85 [1/2M+H]⁺.

### Step 2: Synthesis of 4′-{[4-(32-Amino-3,6,9,12,15,18,21,24,27,30-decaoxadotriacontan-1-yl)piperazin-1-yl]methyl}-N-[(4,6-dimethyl-2-oxo-1H-pyridin-3-yl)methyl]-5-[ethyl(oxan-4-yl)amino]-4-methyl-[1,1′-biphenyl]-3-carboxamide (INT111-515-2)

The procedure was the same as (INT-29-110)). 35.00 mg of tert-butyl N-(32-{4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazin-1-yl}-3,6,9,12,15,18,21,24,27,30-decaoxadotriacontan-1-yl)carbamate was used, 31.00 mg crude of desired product was obtained as yellow oil. LC/MS: mass calcd. for C₅₆H₉₀N₆O₁₃: 1054.65, found: 1055.95 [M+H]⁺.

### Step 3: Synthesis of N-[5-[(3-{[2-({2-[(5-{[2-([2-[(32-[4-[(3′-{[(4,6-dimethyl-2-oxo-1H-pyridin-3-yl)methyl]carbamoyl}-5′-[ethyl(oxan-4-yl)amino]-4′-methyl-[1,1′-biphenyl]-4-yl)methyl]piperazin-1-yl}-3,6,9,12,15,18,21,24,27,30-decaoxadotriacontan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 167)

The procedure was the same as (INT61-04-OH-21), but the reaction mixture was purified by Prep-HPLC. 32.98 mg of 3-[(1-methyl-4-{1-methyl-4-[3-({1-methyl-4-[4methylethyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 5.90 mg of desired product was obtained as light yellow solid (9.18% yield). HRMS: mass calcd. for C₁₀₇H₁₄₉N₂₇O₂₄: 2196.1268, found: 2197.1299 [M+H]⁺.

### Example 49. Synthesis of Compound 171

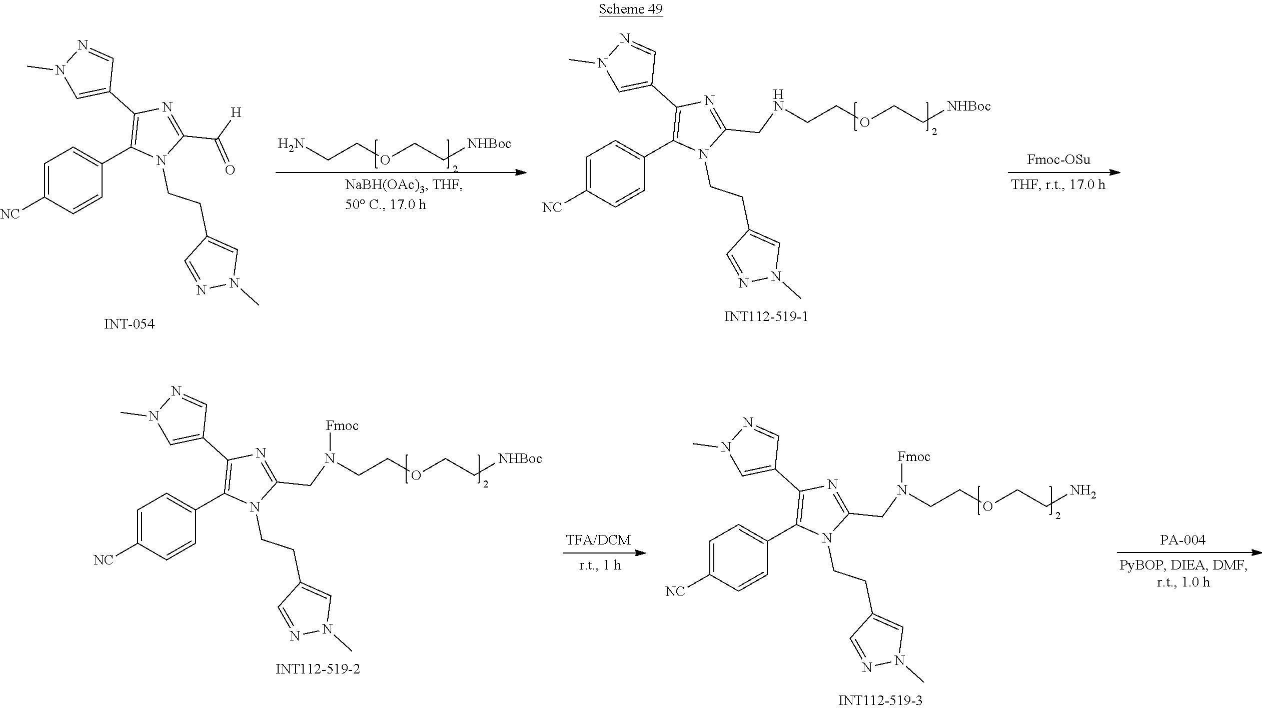

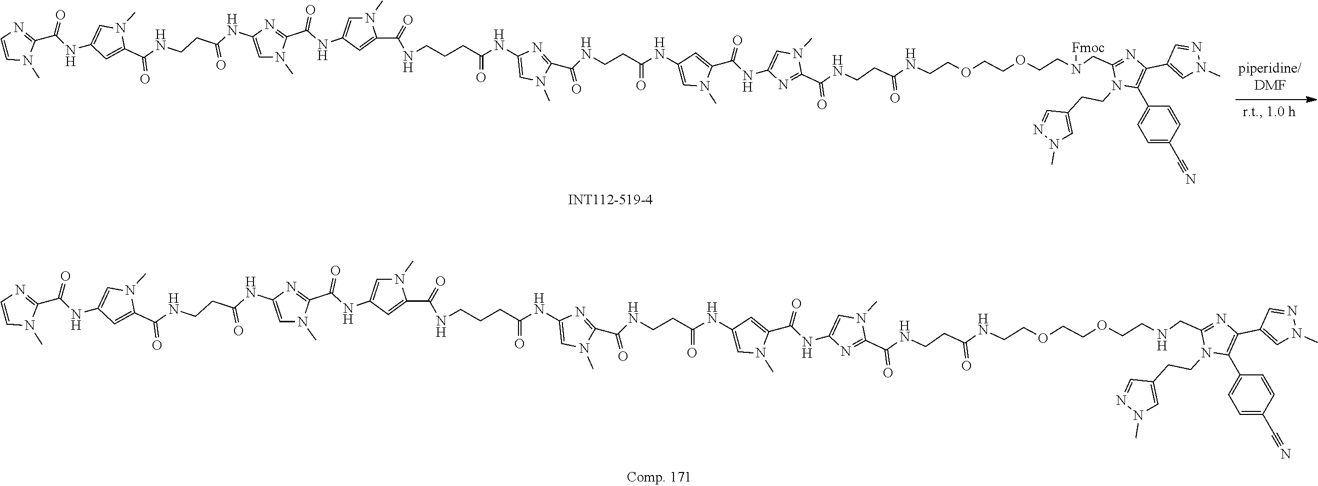

### Step 1: Synthesis of tert-butyl N-(2-{2-[2-({[5-(4-cyanophenyl)-4-(1-methylpyrazol-4-yl)-1-[2-(1-methylpyrazol-4-yl)ethyl]imidazol-2-yl]methyl}amino)ethoxy]ethoxy}ethyl)carbamate (INT112-519-1)

To a stirred solution of 4-[2-formyl-5-(1-methylpyrazol-4-yl)-3-[2-(1-methylpyrazol-4-yl)ethyl]imidazol-4-yl]benzonitrile (100.00 mg, 0.26 mmol, 1.00 equiv) in THF (5.00 mL) was added NaBH(OAc)₃ (164.96 mg, 0.78 mmol, 3.00 equiv) and tert-butyl N-{2-[2-(2-aminoethoxy)ethoxy]ethyl}carbamate (77.31 mg, 0.31 mmol, 1.20 equiv) in portions at room temperature.

The resulting mixture was stirred for 17.0 h at 50° C. The reaction mixture was quenched with saturated NH₄Cl (aq), and the residue was extracted with EA (3×10 mL), dried over Na₂SO₄, filtrated and concentrated. The residue was purified by silica gel column chromatography, eluted with CH₂Cl₂/MeOH (10:1) to afford tert-butyl N-(2-{2-[2-({[5-(4-cyanophenyl)-4-(1-methylpyrazol-4-yl)-1-[2-(1-methylpyrazol-4-yl)ethyl]imidazol-2-yl]methyl}amino)ethoxy]ethoxy}ethyl)carbamate (80.00 mg, 49.91% yield) as yellow oil. LC/MS: mass calcd. For C₃₂H₄₃N₉O₄: 617.34, found: 618.45[M+H]⁺.

### Step 2: Synthesis of tert-butyl N-(2-{2-[2-({[5-(4-cyanophenyl)-4-(1-methylpyrazol-4-yl)-1-[2-(1-methylpyrazol-4-yl)ethyl]imidazol-2-yl]methyl}[(9H-fluoren-9-ylmethoxy)carbonyl]amino)ethoxy]ethoxy}ethyl)carbamate (INT112-519-2)

The procedure was the same as tert-butyl N-[2-(2-{[5-(4-aminophenyl) penta-2,4-diyn-1-yl][(9H-fluoren-9-ylmethoxy)carbonyl]amino}ethoxy)ethyl]carbamate (INT95-419-102). 70.00 mg of tert-butyl N-(2-{2-[2-({[5-(4-cyanophenyl)-4-(1-methylpyrazol-4-yl)-1-[2-(1-methylpyrazol-4-yl)ethyl]imidazol-2-yl]methyl}amino)ethoxy]ethoxy}ethyl)carbamate was used, 120.00 mg of desired product was obtained as yellow solid (126.07% yield). LC/MS: mass calcd. For C₄₇H₅₃N₉O₆: 839.41, found: 840.40[M+H]⁺.

### Step 3: Synthesis of 9H-fluoren-9-ylmethyl N-{2-[2-(2-aminoethoxy)ethoxy] ethyl}-N-{[5-(4-cyanophenyl)-4-(1-methylpyrazol-4-yl)-1-[2-(1-methylpyrazol-4-yl)ethyl]imidazol-2-yl]methyl}carbamate (INT112-519-3)

The procedure was the same as (INT-29-110). 60.00 mg of tert-butyl N-(2-{2-[2-({[5-(4-cyanophenyl)-4-(1-methylpyrazol-4-yl)-1-[2-(1-methylpyrazol-4-yl)ethyl]imidazol-2-yl]methyl}[(9H-fluoren-9-ylmethoxy)carbonyl]amino)ethoxy] ethoxy}ethyl)carbamate was used, 60.00 mg crude of desired product was obtained as yellow oil. LC/MS: mass calcd. For C₄₂H₄₅N₉O₄:739.36, found: 370.95[1/2M+H]⁺.

### Step 4: Synthesis of 9H-fluoren-9-ylmethyl N-{[5-(4-cyanophenyl)-4-(1-methylpyrazol-4-yl)-1-[2-(1-methylpyrazol-4-yl)ethyl]imidazol-2-yl]methyl}-N-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}carbamate (INT112-519-4)

The procedure was the same as (INT61-04-OH-21). 70.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was used, 55.00 mg of desired product was obtained as yellow oil (48.44% yield). LC/MS: mass calcd. For C₉₃H₁₀₄N₃₀O₁₅: 1880.83, found: 942.15[M/2+H]⁺.

### Step 5: Synthesis of N-{5-[(3-{[2-({2-[(5-{[2-({2-[(2-{2-[2-({[5-(4-cyanophenyl)-4-(1-methylpyrazol-4-yl)-1-[2-(1-methylpyrazol-4-yl)ethyl]imidazol-2-yl]methyl}amino)ethoxy]ethoxy}ethyl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 171)

The procedure was the same as tert-butyl N-(5-{4-[(2S)-2-amino-3-[(tert-butyldiphenylsilyl)oxy]propanamido]phenyl}penta-2,4-diyn-1-yl)carbamate (INT94-417-12), but the reaction mixture was purified by Prep-HPLC. 55.00 mg of 9H-fluoren-9-ylmethyl N-{[5-(4-cyanophenyl)-4-(1-methylpyrazol-4-yl)-1-[2-(1-methylpyrazol-4-yl)ethyl]imidazol-2-yl]methyl}-N-{2-[2-(2-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido}ethoxy)ethoxy]ethyl}carbamate was used, 15.50 mg of desired product was obtained as white solid (31.25% yield). HRMS: mass calcd. For C₇₈H₉₄N₃₀O₁₃: 1658.7617, found: 1659.7675[M+H]⁺.

### Example 50. Synthesis of Compound 189

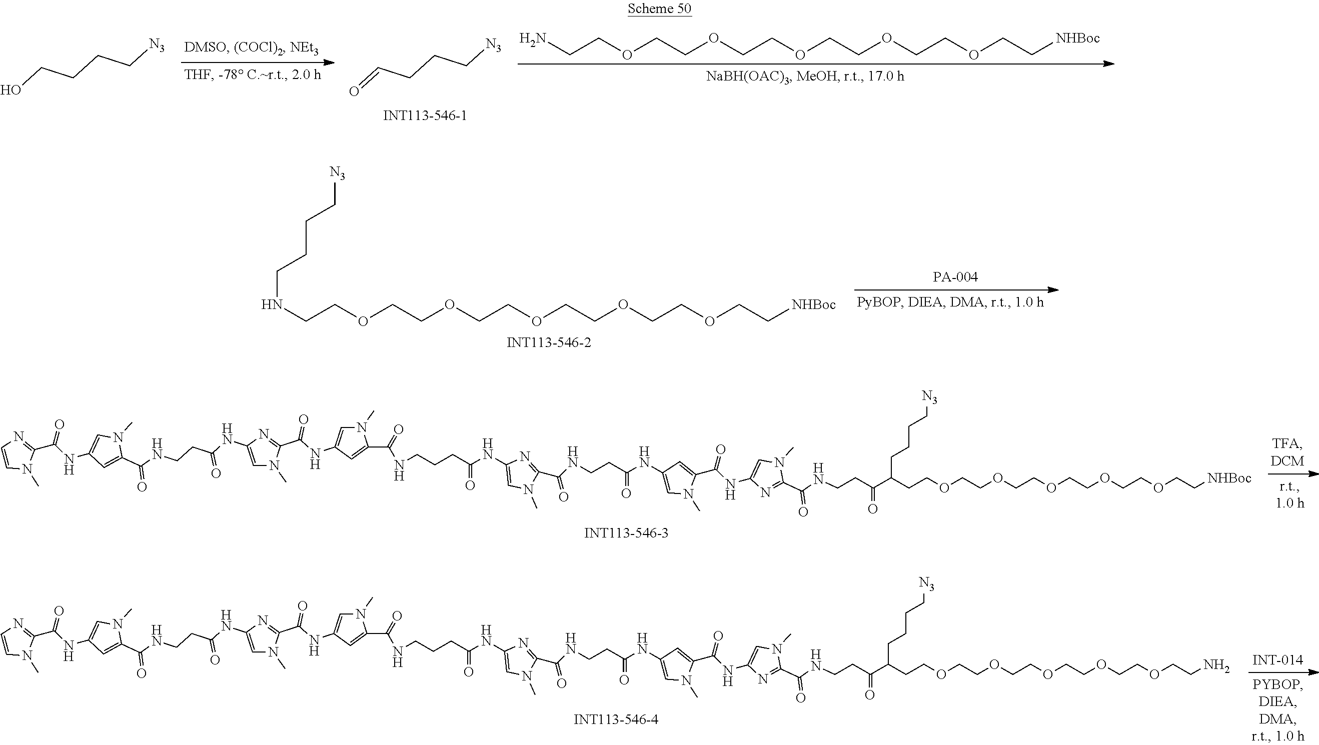

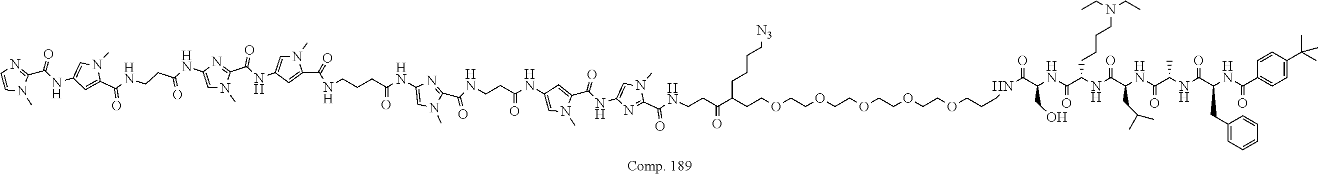 [SMILES: CCN(CC)CCCC[C@H](NC(=O)[C@H](CC(C)C)NC(=O)[C@H](C)NC(=O)[C@H](Cc1ccccc1)NC(=O)c1ccc(C(C)(C)C)cc1)C(=O)N[C@@H](CO)C(=O)NCCCOCCOCCOCCOCCOCCC(CCCCN=[N+]=[N-])C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C]

### Step 1: Synthesis of 4-azidobutanal (INT13-546-1)

1A stirred solution of (COCl)₂ (0.67 mL, 7.82 mmol, 3.00 equiv) in DCM (10.00 mL) was cooled down to −78° C. and DMSO (1.11 mL, 15.636 mmol, 6 equiv) in DCM (10.00 mL) was added dropwise over 2 minutes. The reaction mixture was stirred at −78° C. for 20 minutes, then a solution of 4-azidobutan-1-ol (300.00 mg, 2.61 mmol, 1.00 equiv) in DCM (5.00 mL) was added dropwise. The reaction mixture was stirred another 30 minutes at −78° C., before NEt₃ (2.90 mL, 20.85 mmol, 8.00 equiv) was added dropwise. The reaction mixture was warmed up to 0° C. and stirred for 5 minutes. Saturated aqueous solution of NaHCO₃ (10 mL) was added to quench the reaction. The organic layer was separated and the aqueous layer was extracted with DCM (3×20 mL). The combined organic extracts were washed with brine (20 mL), dried over Na₂SO₄, filtered, and concentrated in vacuum. This resulted in 4-azidobutanal (300.00 mg, crude) as light yellow oil. The crude product was used in the next step directly without further purification. ¹H NMR (300 MHz, CDCl₃) δ: 9.80 (s, 1H), 3.63 (t, J=6.6 Hz, 2H), 2.58 (t, J=6.0 Hz, 2H), 1.81-1.96 (m, 2H).

### Step 2: Synthesis of tert-butyl N-(22-azido-3,6,9,12,15-pentaoxa-18-azadocosan-1-yl)carbamate (INT113-546-2)

To a stirred solution of 4-azidobutanal (300.00 mg, 2.65 mmol, 4.04 equiv) and tert-butyl N-(17-amino-3,6,9,12,15-pentaoxaheptadecan-1-yl)carbamate (250.00 mg, 0.66 mmol, 1.00 equiv) in MeOH (5.00 mL) was added NaBH₃CN (123.87 mg, 1.97 mmol, 3.00 equiv) at room temperature. The resulting mixture was stirred for overnight at room temperature. After reaction, the reaction was quenched by the addition of sat. NH₄Cl (aq.) (10 mL) at 0° C. The resulting mixture was concentrated under vacuum. The resulting mixture was extracted with EtOAc (3×10 mL). The combined organic layers were washed with water (2×10 mL), dried over anhydrous Na₂SO₄. After filtration, the filtrate was concentrated under reduced pressure. The residue was purified by Prep-TLC (CH₂Cl₂/MeOH=12:1) to afford tert-butyl N-(22-azido-3,6,9,12,15-pentaoxa-18-azadocosan-1-yl)carbamate (150.00 mg, 47.80% yield) as a light yellow oil. LC/MS: mass calcd. For C₂₁H₄₃N₅O₇: 477.32, found: 478.20[M+H]⁺.

### Step 3: Synthesis of tert-butyl N-{17-[N-(4-azidobutyl)-3-[(1-methyl-4-[1-methyl-4-[3-([1-methyl-4-[4-([1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido]-3,6,9,12,15-pentaoxaheptadecan-1-yl}carbamate (INT113-546-3)

The procedure was the same as (INT61-04-OH-21), but the solvent was DMA. 170.00 mg of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl) formamido]propanoic acid was used, 130.00 mg of desired product was obtained as light yellow solid (53.54% yield). LC/MS: mass calcd. For C₇₂H₁₀₂N₂₆O₁₈: 1618.79, found: 1619.85[M+H]⁺.

### Step 4: Synthesis of N-[5-[(3-{[2-({2-[(5-{[2-({2-[(17-amino-3,6,9,12,15-pentaoxaheptadecan-1-yl)(4-azidobutyl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (INT113-546-4)

The procedure was the same as (INT-29-110). 160.00 mg of tert-butyl N-{17-[N-(4-azidobutyl)-3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanamido]-3,6,9,12,15-pentaoxaheptadecan-1-yl}carbamate was used, 160.00 mg crude of desired product was obtained a light yellow oil. LC/MS: mass calcd. For C₆₇H₉₄N₂₆O₁₆: 1518.73, found: 761.10[M/2+H]⁺.

### Step 4: Synthesis of N-[5-[(3-{[2-([2-[(5-{[2-([2-[(4-azidobutyl)([17-[(2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanamido]-3,6,9,12,15-pentaoxaheptadecan-1-yl})carbamoyl]ethyl}carbamoyl)-1-methylimidazol-4-yl]carbamoyl}-1-methylpyrrol-3-yl)carbamoyl]ethyl} carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (Comp. 181)

The procedure was the same as (INT61-04-OH-21), but the solvent was DMA and the solid was purified by Prep-HPLC. 70.00 mg of (2S)-2-[(2S)-2-[(2S)-2-[(2S)-2-[(2S)-2-[(4-tert-butylphenyl)formamido]-3-phenylpropanamido]propanamido]-4-methylpentanamido]-6-(diethylamino)hexanamido]-3-hydroxypropanoic acid was used, 97.30 mg of desired product was obtained as white solid (47.40% yield). HRMS: mass calcd. For C₁₀₉H₁₅₆N₃₂O₂₃: 2281.2021, found: 2304.1859 [M+Na]⁺.

### Example 51. Synthesis of Compound 190

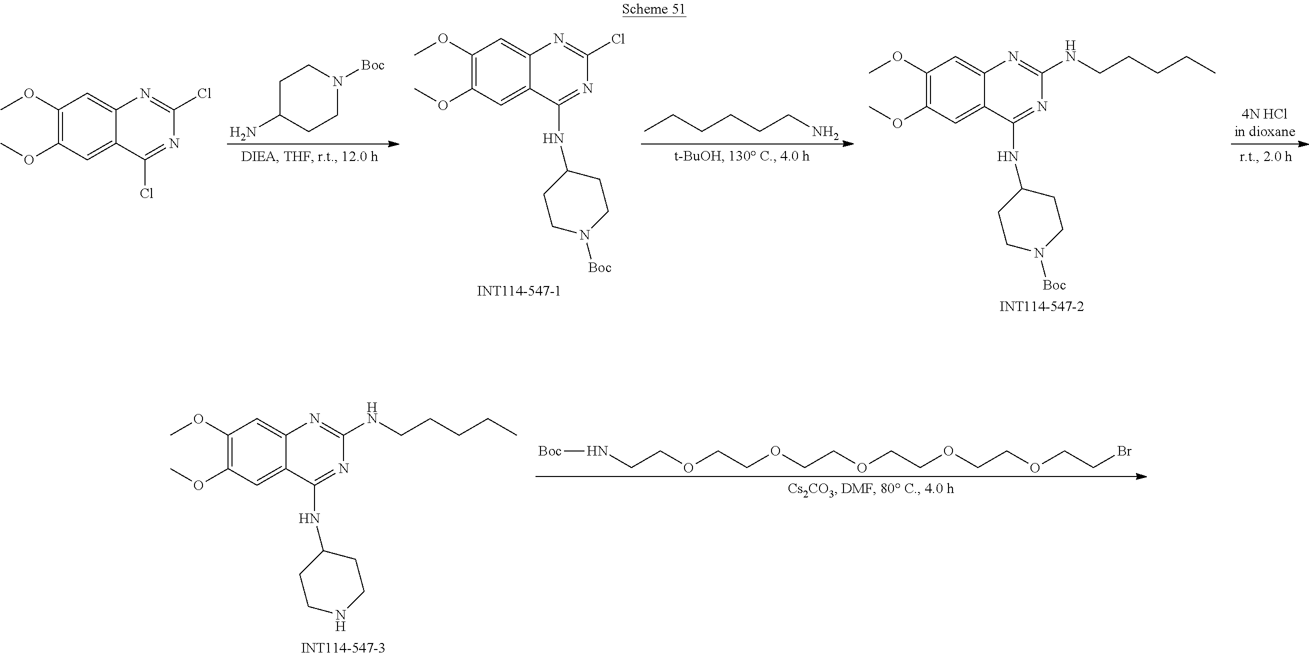

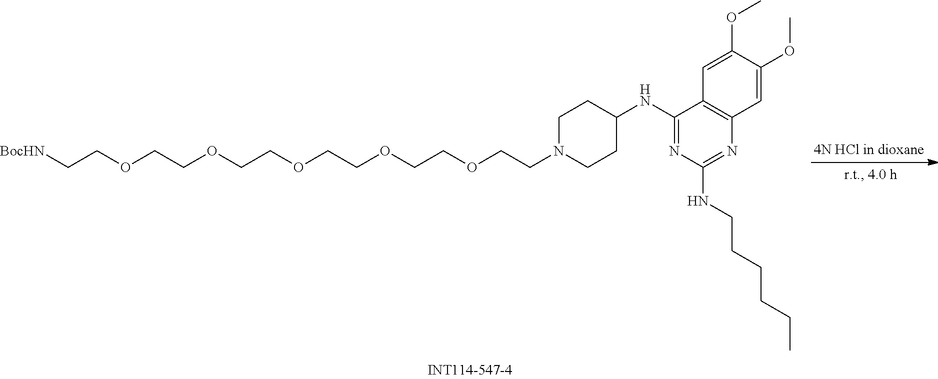 [SMILES: CCCCCCNc1nc(NC2CCN(CCOCCOCCOCCOCCOCCNC(=O)OC(C)(C)C)CC2)c2cc(OC)c(OC)cc2n1]

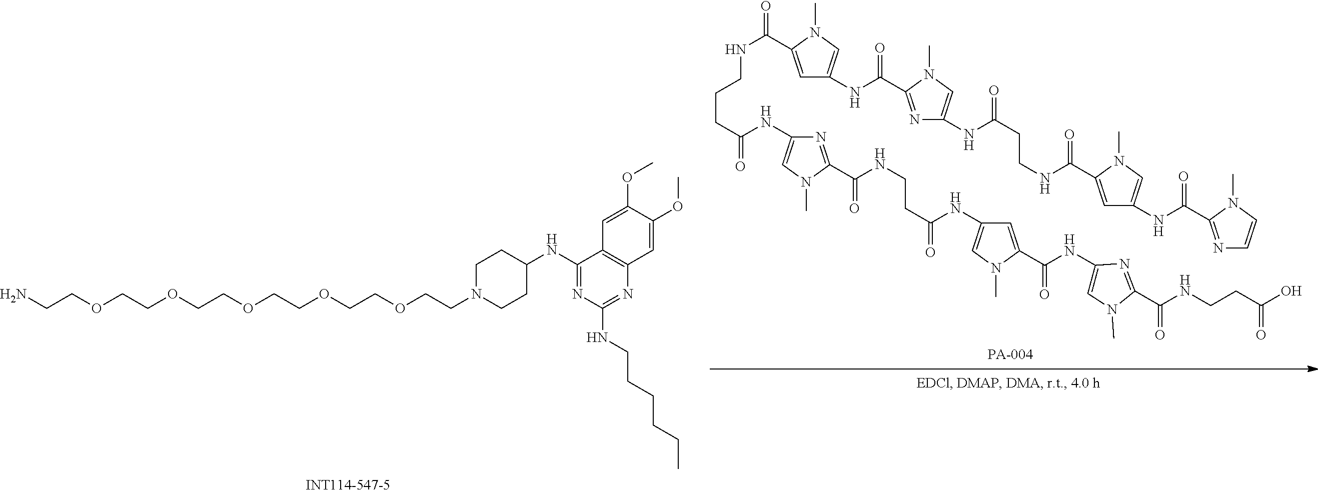 [SMILES: CCCCCCNc1nc(NC2CCN(CCOCCOCCOCCOCCOCCN)CC2)c2cc(OC)c(OC)cc2n1 ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

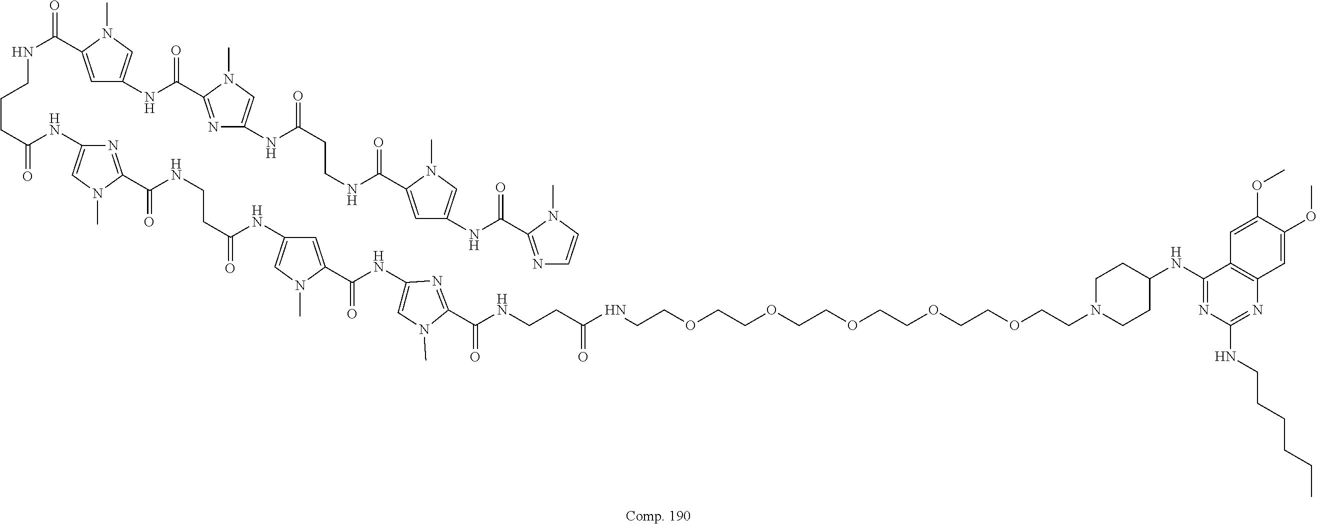 [SMILES: CCCCCCNc1nc(NC2CCN(CCOCCOCCOCCOCCOCCNC(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)CC2)c2cc(OC)c(OC)cc2n1]

### Step 1: Synthesis of tert-butyl 4-[(2-chloro-6,7-dimethoxyquinazolin-4-yl)amino]piperidine-1-carboxylate (INT114-547-1)

To a stirred mixture of 2,4-dichloro-6,7-dimethoxyquinazoline (3.00 g, 11.58 mmol, 1 equiv) in THF (50 mL, 617.15 mmol, 53.30 equiv) was added tert-butyl 4-aminopiperidine-1-carboxylate (5.10 g, 25.47 mmol, 2.2 equiv) and DIEA (1.80 g, 13.89 mmol, 1.2 equiv) at r.t. The resulting mixture was stirred for 12.0 h at r.t. The resulting mixture was filtered, the filter cake was washed with THF (50 mL×2). The filtrate was concentrated under reduced pressure. The residue was purified by silica gel column chromatography, eluted with PE/EA. 5.00 g of desired product was obtained as light yellow solid (91.90% yield). LC/MS: mass calcd. For C₂₀H₂₇ClN₄O₄: 422.17, found: 423.19 [M+H]⁺.

### Step 2: Synthesis of tert-butyl 4-{[2-(hexylamino)-6,7-dimethoxyquinazolin-4-yl[amino}piperidine-1-carboxylate (INT114-547-2)

To a stirred mixture of tert-butyl 4-[(2-chloro-6,7-dimethoxyquinazolin-4-yl)amino]piperidine-1-carboxylate (700 mg, 1.655 mmol, 1 equiv) in t-BuOH (7 mL, 73.663 mmol, 44.50 equiv) was added hexylamine (1674.95 mg, 16.550 mmol, 10 equiv) at room temperature. The resulting mixture was stirred for 4.0 h at 130° C. The resulting mixture (combined with EB2104506-100P1 and EB2104506-101P1) was concentrated under reduced pressure. The residue was purified by silica gel column chromatography, eluted with PE/EA. 2.10 g of desired product was obtained as light yellow solid (86.72% yield). LC/MS: mass calcd. For C₂₆H₄₁N₅O₄: 487.32, found: 488.30 [M+H]⁺.

### Step 3: Synthesis of N2-hexyl-6,7-dimethoxy-N4-(piperidin-4-yl)quinazoline-2,4-diamine (INT114-547-3)

The procedure was the same as (INT91-010-51). 1.90 g of tert-butyl 4-{[2-(hexylamino)-6,7-dimethoxyquinazolin-4-yl]amino}piperidine-1-carboxylate was used, 1.70 g of desired product was obtained as light yellow solid (101.33% yield). LC/MS: mass calcd. For C₂₁H₃₃N₅O₂: 387.53, found: 388.20 [M+H]⁺.

### Step 4: Synthesis of tert-butyl N-[17-(4-{[2-(hexylamino)-6,7-dimethoxyquinazolin-4-yl[amino}piperidin-1-yl)-3,6,9,12,15-pentaoxaheptadecan-1-yl]carbamate (INT114-547-4)

The procedure was the same as tert-butyl N-(17-[4-[(5-[[(5-tert-butyl-1,3-oxazol-2-yl)methyl]sulfanyl]-1,3-thiazol-2-yl)carbamoyl]piperidin-1-yl]-3,6,9,12,15-pentaoxaheptadecan-1-yl)carbamate (INT91-010-50), but the reaction base was Cs₂CO₃ and the reaction time was 4.0 h. 300.00 mg of N2-hexyl-6,7-dimethoxy-N4-(piperidin-4-yl)quinazoline-2,4-diamine was used, 330.00 mg of desired product was obtained as light yellow oil (56.76% yield). LC/MS: mass calcd. For C₃₈H₆₆N₆O₉: 750.49, found: 751.35 [M+H]⁺.

### Step 5: Synthesis of N4-[1-(17-amino-3,6,9,12,15-pentaoxaheptadecan-1-yl) piperidin-4-yl]-N2-hexyl-6,7-dimethoxyquinazoline-2,4-diamine (INT114-547-5)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51), but the reaction time was 4.0 h. 320.00 mg of tert-butyl N-[17-(4-{[2-(hexylamino)-6,7-dimethoxyquinazolin-4-yl]amino}piperidin-1-yl)-3,6,9,12,15-pentaoxaheptadecan-1-yl]carbamate was used, 274.00 mg of desired product was obtained as yellow oil (98.80% yield). LC/MS: mass calcd. For C₃₂H₅₆N₆O₇: 650.44, found: 651.35 [M+H]⁺.

### Step 6: Synthesis of N-(5-{[3-({2-[(2-{[5-({2-[(2-{[17-(4-{[2-(hexylamino)-6,7-dimethoxyquinazolin-4-yl]amino}piperidin-1-yl)-3,6,9,12,15-pentaoxaheptadecan-1-yl]carbamoyl}ethyl)carbamoyl]-1-methylimidazol-4-yl}carbamoyl)-1-methylpyrrol-3-yl]carbamoyl}ethyl)carbamoyl]-1-methylimidazol-4-yl}carbamoyl)propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Comp. 190)

The procedure was the same as (INT61-025-20). 36.46 mg of N4-[1-(17-amino-3,6,9,12,15-pentaoxaheptadecan-1-yl)piperidin-4-yl]-N2-hexyl-6,7-dimethoxyquinazoline-2,4-diamine was used, 3.10 mg of desired product was obtained as a white solid (4.01% yield). HRMS: mass calcd. For C₈₄H₁₁₇N₂₇O₁₈: 1791.91, found: 1792.91 [M+H]⁺.

### Example 52. Synthesis of Compound 192

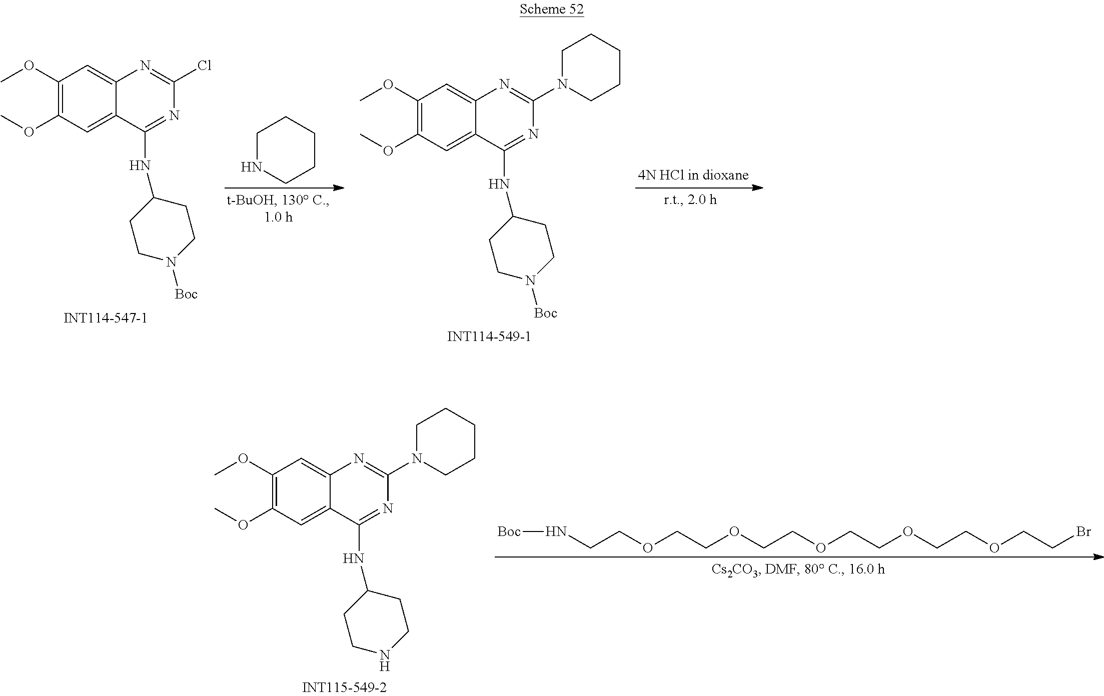

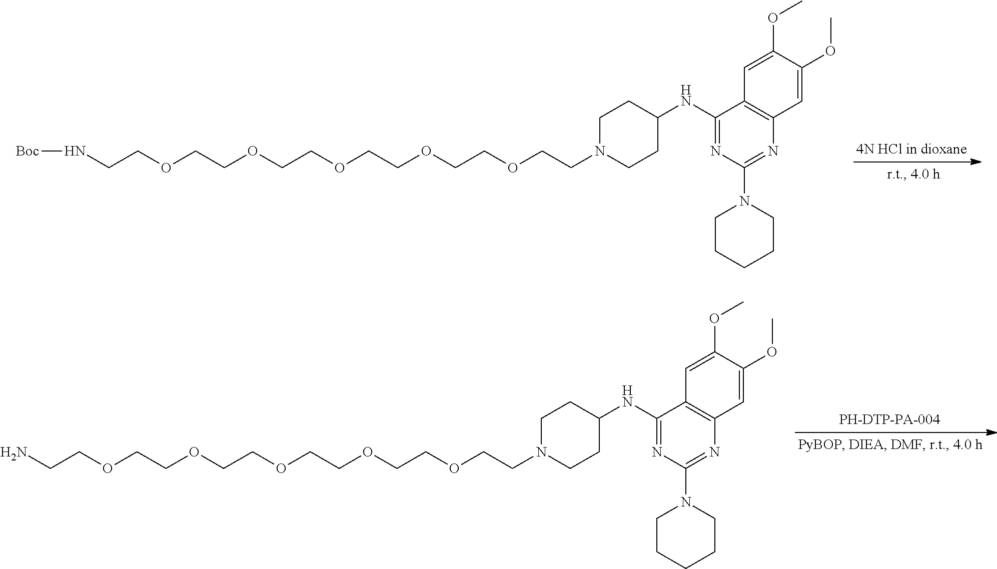

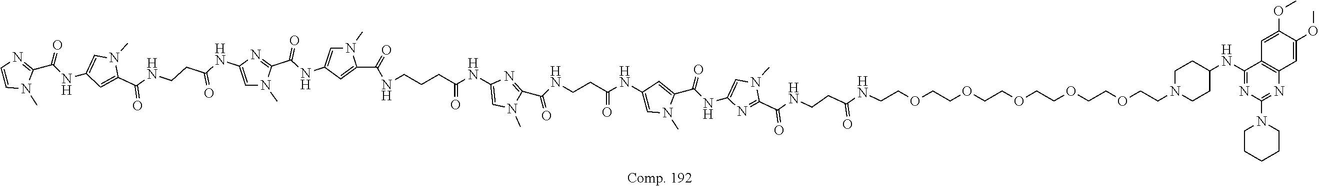 [SMILES: COc1cc2nc(N3CCCCC3)nc(NC3CCN(CCOCCOCCOCCOCCOCCNC(=O)CCNC(=O)c4nc(NC(=O)c5cc(NC(=O)CCNC(=O)c6nc(NC(=O)CCCNC(=O)c7cc(NC(=O)c8nc(NC(=O)CCNC(=O)c9cc(NC(=O)c%10nccn%10C)cn9C)cn8C)cn7C)cn6C)cn5C)cn4C)CC3)c2cc1OC]

### Step 1: Synthesis of tert-butyl 4-{[6,7-dimethoxy-2-(piperidin-1-yl)quinazolin-4-yl[amino}piperidine-1-carboxylate (INT115-549-1)

The procedure was the same as tert-butyl 4-{[2-(hexylamino)-6,7-dimethoxyquinazolin-4-yl]amino}piperidine-1-carboxylate. (INT114-547-2), but the reaction time was 1.0 h. 700.00 mg of tert-butyl 4-[(2-chloro-6,7-dimethoxyquinazolin-4-yl)amino]piperidine-1-carboxylate was used, 1.50 g of desired product was obtained as an off-white solid (64.05% yield). LC/MS: mass calcd. For C₂₅H₃₇N₅O₄: 471.28, found: 472.20 [M+H]⁺.

### Step 2: Synthesis of 6,7-dimethoxy-2-(piperidin-1-yl)-N-(piperidin-4-yl)quinazolin-4-amine (INT115-549-2)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51). 1.40 g of tert-butyl 4-{[6,7-dimethoxy-2-(piperidin-1-yl)quinazolin-4-yl]amino}piperidine-1-carboxylate was used, 1.60 g of desired product was obtained as light yellow solid (130.58% yield). LC/MS: mass calcd. For C₂₀H₂₉N₅O₂: 371.23, found: 372.15 [M+H]⁺.

### Step 3: Synthesis of tert-butyl N-[17-(4-{[6,7-dimethoxy-2-(piperidin-1-yl)quinazolin-4-yl[amino}piperidin-1-yl)-3,6,9,12,15-pentaoxaheptadecan-1-yl]carbamate (INT115-549-3)

The procedure was the same as tert-butyl N-(17-[4-[(5-[[(5-tert-butyl-1,3-oxazol-2-yl)methyl]sulfanyl]-1,3-thiazol-2-yl)carbamoyl]piperidin-1-yl]-3,6,9,12,15-pentaoxaheptadecan-1-yl)carbamate (INT91-010-50), but the reaction base was Cs₂CO₃ and the reaction time was 4.0 h. 300.00 mg of 6,7-dimethoxy-2-(piperidin-1-yl)-N-(piperidin-4-yl)quinazolin-4-amine was used, 440.00 mg of desired product was obtained as light yellow oil (74.13% yield). LC/MS: mass calcd. For C₃₇H₆₂N₆O₉: 734.46, found: 735.35 [M+H]⁺.

### Step 4: Synthesis of N-[1-(17-amino-3,6,9,12,15-pentaoxaheptadecan-1-yl)piperidin-4-yl]-6,7-dimethoxy-2-(piperidin-1-yl)quinazolin-4-amine (INT115-549-4)

The procedure was the same as 1-(17-amino-3,6,9,12,15-pentaoxaheptadecan-1-yl)-N-(5-[[(5-tert-butyl-1,3-oxazol-2-yl)methyl]sulfanyl]-1,3-thiazol-2-yl)piperidine-4-carboxamide (INT91-010-51), but the reaction time was 4.0 h. 430.00 mg of tert-butyl N-[17-(4-{[6,7-dimethoxy-2-(piperidin-1-yl) quinazolin-4-yl]amino}piperidin-1-yl)-3,6,9,12,15-pentaoxaheptadecan-1-yl]carbamate was used, 50.00 mg of desired product was obtained as yellow oil (13.46% yield). LC/MS: mass calcd. For C₃₂H₅₄N₆O₇: 634.41, found: 635.30 [M+H]⁺.

### Step 5: Synthesis of N-(5-{[3-([2-[(2-{[5-([2-[(2-{[17-(4-{[6,7-dimethoxy-2-(piperidin-1-yl)quinazolin-4-yl]amino}piperidin-1-yl)-3,6,9,12,15-pentaoxaheptadecan-1-yl]carbamoyl}ethyl)carbamoyl]-1-methylimidazol-4-yl}carbamoyl)-1-methylpyrrol-3-yl]carbamoyl}ethyl)carbamoyl]-1-methylimidazol-4-yl}carbamoyl)propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Comp. 192)

The procedure was the same as (INT61-04-OH-21), but the reaction time was 4.0 h and the reaction mixture was purified by Prep-HPLC. 32.83 mg of N-[1-(17-amino-3,6,9,12,15-pentaoxaheptadecan-1-yl)piperidin-4-yl]-6,7-dimethoxy-2-(piperidin-1-yl)quinazolin-4-amine was used, 1.70 mg of desired product was obtained as a white solid (2.22% yield). LC/MS: mass calcd. For C₈₃H₁₁₃N₂₇O₁₈: 1775.88, found: 1776.87 [M+H]⁺.

### Example 53. Synthesis of Additional Compounds of the Disclosure

The compounds of the application were made by the methods similar to Examples 1-52. A summary of the analytical data is represented in Table 7.

TABLE 7
Mass spectrometry data for the
compound of the disclosure.

| | | Observed |
|---|---|---|
| | | [M + H]+ from |
| Cmpd. | | TOF-HRMS |
| No. | Calc. Mass | [m/z] |
| 2 | 1796.8125 | 1797.817 |
| 10 | 1976.0420 | 1977.0458 |
| 11 | 2196.1730 | 2197.1851 |
| 17 | 2004.9085 | 2005.9215 |
| 25 | 2184.1380 | 2185.1462 |
| 26 | 2404.2690 | 2405.2712 |
| 27 | 2624.4000 | 2625.4111 |
| 53 | 2073.0082 | 2074.0100 |
| 54 | 2293.1392 | 2294.1487 |
| 55 | 2107.0904 | 2108.0987 |
| 56 | 2056.0795 | 2057.0866 |
| 57 | 1985.0424 | 1986.0515 |
| 58 | 2159.1217 | 2160.1263 |
| 59 | 2236.1694 | 2237.1775 |
| 60 | 1738.7614 | 1739.7668 |
| 61 | 2179.0235 | 2180.0291 |
| 62 | 1851.8255 | 1852.83 |
| 63 | 2292.0876 | 2293.0969 |
| 64 | 2052.0595 | 2053.0669 |
| 65 | 1734.8703 | 1735.8748 |
| 66 | 1998.0224 | 1999.0343 |
| 67 | 1786.9016 | 1787.9117 |
| 68 | 2050.0537 | 2051.0701 |
| 69 | 2008.0333 | 2009.0449 |
| 70 | 1964.0070 | 1965.0139 |
| 71 | 2049.0962 | 2050.1128 |
| 72 | 2140.1119 | 2141.1141 |
| 73 | 2096.0857 | 2097.1038 |
| 74 | 2066.0751 | 2067.0925 |
| 75 | 2066.0751 | 2067.0896 |
| 76 | 2080.0908 | 2081.1089 |
| 77 | 2104.0908 | 2105.1079 |
| 78 | 1975.0118 | 1976.0172 |
| 79 | 1852.9638 | 1853.988 |
| 80 | 1602.7916 | 1603.7958 |
| 81 | 1865.9438 | 1866.9565 |
| 82 | 1924.0009 | 1924.9993 |
| 83 | 1976.0322 | 1977.0362 |
| 84 | 2108.1108 | 2109.1265 |
| 85 | 2142.0912 | 2143.1074 |
| 86 | 2142.0912 | 2143.0967 |
| 87 | 2128.0755 | 2129.0901 |
| 88 | 2014.0326 | 2015.0364 |
| 89 | 2014.0326 | 2015.0276 |
| 90 | 2000.0169 | 2001.0326 |
| 91 | 1802.8277 | 1803.8384 |
| 92 | 1817.8386 | 1818.8487 |
| 93 | 1860.8444 | 1861.8563 |
| 94 | 1788.8121 | 1789.8268 |
| 95 | 1846.8288 | 1847.8364 |
| 96 | 2074.0227 | 2075.0222 |
| 98 | 2161.0911 | 2162.106 |
| 99 | 2024.0129 | 2025.0278 |
| 100 | 2076.0442 | 2077.0443 |
| 101 | 2199.1490 | 2200.1454 |
| 102 | 2241.1596 | 2242.1768 |
| 103 | 2113.1010 | 2114.0976 |
| 104 | 2113.1010 | 2136.0826[M + Na] |
| 105 | 2164.1119 | 2165.0991 |
| 106 | 2164.1119 | 2165.1045 |
| 107 | 2041.0686 | 2042.0809 |
| 108 | 2056.0795 | 2057.0675 |
| 109 | 2098.0901 | 2099.0925 |
| 110 | 1919.0206 | 1920.028 |
| 111 | 1934.0315 | 1935.0288 |
| 112 | 1976.0421 | 1977.0352 |
| 113 | 2041.0686 | 2042.0631 |
| 114 | 2098.0901 | 2121.0710[M + Na] |
| 115 | 1919.0206 | 1920.042 |
| 116 | 1934.0315 | 1935.0486 |
| 117 | 1976.0421 | 1977.0579 |
| 118 | 2027.0431 | 2028.0412 |
| 119 | 2379.2528 | 2380.2722 |
| 120 | 1656.7191 | 1657.7342 |
| 121 | 2096.9812 | 2097.9786 |
| 122 | 1724.8185 | 1725.8298 |
| 123 | 1608.6984 | 1631.6928[M + Na] |
| 124 | 1784.8033 | 1785.805 |
| 125 | 1680.7559 | 1681.7613 |
| 126 | 1856.8608 | |
| 127 | 1710.7665 | 1711.7682 |
| 128 | 1886.8713 | 1887.8745 |
| 129 | 1684.7859 | 1685.7944 |
| 130 | 2235.1490 | 2258.1210[M + Na] |
| 131 | 2250.1599 | 2251.1658 |
| 132 | 2292.1705 | 2293.1885 |
| 133 | 2306.1861 | 2307.1819 |
| 134 | 2308.1654 | 2309.183 |
| 135 | 2306.1861 | 2307.1863 |
| 136 | 1992.0370 | 1993.0333 |
| 137 | 2255.1752 | 2256.1672 |
| 138 | 1996.0431 | 1997.0598 |
| 139 | 1994.0639 | 1995.0809 |
| 140 | 2042.0486 | 2043.0612 |
| 141 | 2040.0694 | 2041.0672 |
| 142 | 1801.8437 | 1824.8310[M + Na] |
| 143 | 1859.8604 | 1874.8405 |
| 144 | 2092.1007 | 2093.1174 |
| 145 | 2017.0799 | 2018.0895 |
| 146 | 1826.8918 | 1827.8891 |
| 147 | 1876.8744 | 1877.8772 |
| 148 | 2389.2848 | 2412.2570[M + Na] |
| 149 | 2347.2378 | 2348.2263 |
| 150 | 2020.0180 | 2021.0159 |
| 151 | 2018.0387 | 2019.0378 |
| 152 | 2041.0183 | 2042.0357 |
| 155 | 2031.0340 | 2032.035 |
| 157 | 1792.9121 | 1793.9171 |
| 158 | 1647.8382 | 1648.8407 |
| 159 | 2050.9438 | 2051.9442 |
| 163 | 1697.8287 | 1698.8281 |
| 164 | 1825.9430 | 1826.941 |
| 165 | 1843.9172 | 1844.936 |
| 166 | 1975.9958 | 1977.009 |
| 167 | 2196.1269 | 2197.1299 |
| 168 | 1601.6371 | 1602.6493 |
| 169 | 1733.7157 | 1734.7275 |
| 170 | 1953.8468 | 1954.8525 |
| 171 | 1658.7617 | 1659.7675 |
| 172 | 1790.8403 | 1791.8493 |
| 173 | 2010.9714 | 2011.9735 |
| 174 | 1826.8918 | 1827.8945 |
| 175 | 1876.8744 | 1877.8782 |
| 177 | 2232.1381 | 2233.1377 |
| 178 | 2232.1381 | 2167.0661[M + Na] |
| 179 | 2100.0595 | 2101.0627 |
| 180 | 2295.2218 | 2296.2236 |
| 181 | 2207.1694 | 2208.1693 |
| 184 | 2292.2432 | 2315.2206[M + Na] |
| 185 | 2209.1697 | 2210.1804 |
| 186 | 2292.2432 | 2293.2448 |
| 187 | 2209.1697 | 2210.1684 |
| 189 | 2281.2021 | 2304.1859[M + Na] |
| 190 | 1791.9070 | 1792.9101 |
| 191 | 2012.0381 | 2013.0392 |
| 192 | 1775.8757 | 1776.8777 |
| 193 | 1996.0068 | 1997.0065 |
| 262 | 1983.8163 | 1984.8254 |
| 263 | 1899.7819 | 1900.7826 |
| 264 | 2091.9392 | 2092.9497 |
| 265 | 2027.8405 | 2028.8478 |
| 275 | 2184.1381 | 2185.1341 |
| 285 | 2184.1381 | 2185.1416 |

### BIOLOGICAL EXAMPLES

### Example B1. Biological Assays

The methods as set forth below will be used to demonstrate the binding of the disclosed molecules and the efficacy in treatment. In general, the assays are directed at evaluating the effect of the disclosed molecules on the level of expression of the target gene containing CAG or CTG repeats (e.g., dmpk, atxn1, atxn2, atxn3, cacna1a, atxn7, ppp2r2b, tbp, htt, jph3, ar, atn, and gene encoding TCF4.

Gene Expression

Expression of a target gene containing CAG or CTG repeats will be assayed by techniques known in the field. These assays include, but are not limited to quantitative reverse transcription polymerase chain reaction (RT-PCR), microarray, or multiplexed RNA sequencing (RNA-seq), with the chosen assay measuring either total expression, or the allele specific expression of the target gene. Exemplary assays are found at: Freeman W M et al., “Quantitative RT-PCR: pitfalls and potential”, BioTechniques 1999, 26, 112-125; Dudley A M et al, “Measuring absolute expression with microarrays with a calibrated reference sample and an extended signal intensity range”, PNAS USA 2002, 99(11), 7554-7559; Wang Z et al., “RNA-Seq: a revolutionary tool for transcriptomics” Nature Rev. Genetics 2009, 10, 57-63.

Production of the translation product of the target gene will be assayed by techniques known in the field. These assays include, but are not limited to Western blot assay, with the chosen assay measuring either total protein expression, or allele specific expression of the target gene.

For use in assay, two tissue models and two animal models are contemplated.

Disease Model I: Human Cell Culture

This model will constitute patient-derived cells, including fibroblasts, induced pluripotent stem cells and cells differentiated from stem cells. Attention will be made in particular to cell types that show impacts of the disease, e.g., neuronal cell types.

Disease Model II: Murine Cell Culture

This model will constitute cell cultures from mice from tissues that are particularly responsible for disease symptoms, which will include fibroblasts, induced pluripotent stem cells and cells differentiated from stem cells and primary cells that show impacts of the disease, e.g., neuronal cell types.

Disease Model III: Murine

This model will constitute mice whose genotypes contain the relevant number of repeats for the disease phenotype—these models should show the expected altered gene expression (e.g., modulation of expression of the target gene.

Disease Model IV: Murine

This model will constitute mice whose genotypes contain a knock in of the human genetic locus from a diseased patient—these models should show the expected altered gene expression (e.g., modulation of expression of the target gene.

All references, patents or applications, U.S. or foreign, cited in the application are hereby incorporated by reference as if written herein in their entireties. Where any inconsistencies arise, material literally disclosed herein controls.

### Example B2. EC₅₀ Assay

DM1 foci reduction assay methods: Myotonic Dystrophy 1 affected patient fibroblasts (Coriell GM04602; 1600 CTG repeats) and wild type fibroblasts (Coriell GM07492; control line) were cultured separately in Gibco DMEM (1×)+4.5 g/L D-Glucose+L-Glutamine+110 mg/L Sodium Pyruvate, supplemented with 10% FBS and 1× Pen/Strep. Cells were maintained in incubator at 37° C. and 5% CO₂ with media refresh every 48-72 hours.

At 90-95% confluency, both cell lines were harvested using Trypl-E then pelleted at 500×g for 5 minutes and were resuspended in media. DM1 fibroblasts were seeded in Agilent 96 well black plates at a density of 5,000 cells/well in 200 μL media; 8 wells were reserved for control fibroblasts. Plates were returned to incubator for 24 hours at 37° C. and 5% CO₂.

Compounds were diluted from 10 mM stock to 1 mM in DMSO, then diluted once more to 6 μM (2× concentration) in media. Media was removed from all plates and cells were replenished with 100 μL media. Cells were treated in 8-point dose response, 1:3 fold dilution, 3 μM top dose via addition of 100 μL of 6 μM (2× concentration) compound to the 100 μL media with cells. Plates were returned to incubator for 48-hour time-course experiment at 37° C. and 5% CO₂.

Following treatment, compounds were removed, and plates were washed with PBS, then cells were fixed in 75 μL 4% PFA solution for 20 minutes at room temperature. Plates were washed twice with PBS and twice with cold 70% ethanol before permeabilization with 250 μL cold 70% ethanol for 24-72 hours at −20° C.

After permeabilization, plates were washed once with a 30% formamide and 2×SSC buffer and rehydrated in that buffer for 15 minutes at room temperature. Cells were incubated overnight at 37° C. in 75 μL of a hybridization solution containing 30% formamide, 2×SSC, 25 mg/mL dextran sulfate, 2.5 mg/mL BSA, 0.2 μg/mL Herring sperm DNA, 2 mM vanadyl-ribonucleoside complex, and 5 nM CAG10-Cy3 probe.

Plates were washed once with 30% formamide in 2×SSC buffer, then twice with the buffer for 30 minutes at 37° C., 300 RPM in an incubating plate shaker. Cells were stained with 75 μL of 2.5 μg/mL DAPI in PBS for 5 minutes at room temperature. Plates were then washed twice with PBS and stored in 250 μL PBS. Plates were sealed with adhesive foil and wiped down with 70% ethanol.

Cells were imaged on a Cytation5 with a 20× objective sampling from 4 areas of each well. Nuclei were captured under DAPI channel and foci under RFP channel. Plates were analyzed on an average foci per nucleus per well basis. Active compounds were defined as those that showed a significant decrease in foci per nucleus from the negative control cells in a dose-responsive manner.

Foci reduction in FECD: F35T cells were cultured in media containing Opti-MEM (ThermoFisher) supplemented with 8% FBS, 20 μg/mL ascorbic acid, 200 mg/mL CaCl₂, 0.08% chondroitin sulfate, 1× Pen/Strep, 100 μg/mL bovine pituitary extract, 5 ng/mL epidermal growth factor, and 20 ng/mL nerve growth factor. Throughout the culture, cells were maintained in an incubator at 37° C. and 5% CO₂. Media was refreshed every 48 hours. Once cells reached adequate confluency, they were harvested and seeded in 96 well plates with a density of 5000 cells per well in 200 μL of the supplemented Opti-MEM media. Cells were returned to the incubator and left to settle for 24 hours at 37° C. and 5% CO₂. Cells were then treated in 8-point dose response with compounds or negative controls and incubated for 48 hours at 37° C. and 5% CO₂. After treatment, cells were fixed with 4% PFA for 20 minutes at room temperature, followed by permeabilization with 70% ethanol. Cells were incubated at −20° C. for a minimum of 1 hour and maximum of 72 hours, after which the ethanol was removed, and cells were washed with PBS. Cells were rehydrated with 30% formamide and 2×SSC buffer for 10 minutes at room temperature. Cells were then incubated overnight at 37° C. in the hybridization solution containing 30% formamide, 2×SSC, 55 mg/mL dextran sulfate, 2.75 mg/mL bovine serum albumin, 0.2 μg/mL Herring sperm DNA, 1% vanadyl-ribonucleoside complex, and 0.05% 10 μM CAG10-Cy3 probe. Cells were washed twice with 30% formamide in 2×SSC, incubating the cells while shaking with the second wash at 37° C. and 200 rpm for 60 minutes. Cells were stained with 5 mg/mL DAPI 1:1000 in PBS, incubating at room temperature for 5 minutes. Cells were then washed with PBS and sealed with adhesive foil, with each well containing a final volume of 150 μL PBS. Cells were imaged on a Cytation 5 and analyzed on a foci per nucleus basis. Active compounds were defined as those that showed a significant decrease in foci per nucleus from the negative control cells in a dose-responsive manner.

Representative in vitro biochemical data is presented in Table 8. A<100 nM; B is 100 nM to 500 nM; C>500 nM.

TABLE 8

| | Foci Reduction in | |
|---|---|---|
| | Fibroblast | |
| Cmpd. | EC_{50, 48 h} | |
| No. | (nM) | Eₘᵢₙ, _{48 h} |
| 2 | A | 0.60 |
| 10 | C | 1.04 |
| 11 | N/A | 1.18 |
| 17 | C | 0.93 |
| 25 | B | 0.11 |
| 26 | B | 0.18 |
| 27 | A | 0.39 |
| 53 | C | N/A |
| 54 | C | N/A |
| 55 | C | 1.08 |
| 56 | C | N/A |
| 57 | C | N/A |
| 58 | C | N/A |
| 59 | B | 0.29 |
| 60 | B | 0.69 |
| 61 | C | N/A |
| 62 | C | 0.75 |
| 63 | C | 0.44 |
| 64 | A | 0.08 |
| 65 | C | 0.20 |
| 66 | B | 0.08 |
| 67 | B | 0.31 |
| 68 | B | 0.27 |
| 69 | A | 0.58 |
| 70 | A | 0.51 |
| 71 | C | 0.41 |
| 72 | A | 0.18 |
| 73 | A | 0.17 |
| 74 | A | 0.15 |
| 75 | A | 0.29 |
| 76 | A | 0.17 |
| 77 | A | 0.36 |
| 78 | C | 1.20 |
| 79 | C | 0.89 |
| 80 | B | 0.50 |
| 81 | A | 0.35 |
| 82 | C | 1.02 |
| 83 | C | 0.94 |
| 84 | C | 0.68 |
| 85 | A | 0.01 |
| 86 | A | 0.08 |
| 87 | A | 0.08 |
| 88 | C | 0.97 |
| 89 | C | 0.93 |
| 90 | C | 0.97 |
| 91 | C | N/A |
| 92 | C | N/A |
| 93 | C | N/A |
| 94 | C | N/A |
| 95 | C | N/A |
| 96 | A | 0.18 |
| 98 | C | 0.97 |
| 99 | A | 0.14 |
| 100 | B | 0.24 |
| 101 | B | 0.04 |
| 102 | B | 0.05 |
| 103 | A | 0.07 |
| 104 | C | 1.08 |
| 105 | C | 1.17 |
| 106 | C | 1.19 |
| 107 | C | 0.19 |
| 108 | B | 0.74 |
| 109 | C | 0.97 |
| 110 | C | N/A |
| 111 | C | 0.93 |
| 112 | C | 0.89 |
| 113 | C | 1.31 |
| 114 | C | 0.87 |
| 115 | C | N/A |
| 116 | C | 0.97 |
| 117 | C | 1.06 |
| 118 | C | 0.92 |
| 119 | C | 1.15 |
| 120 | C | N/A |
| 121 | C | 0.97 |
| 122 | C | 0.93 |
| 123 | C | 0.58 |
| 124 | C | 0.51 |
| 125 | C | 1.01 |
| 126 | C | 0.94 |
| 127 | C | 0.89 |
| 128 | C | 0.88 |
| 129 | C | 1.04 |
| 130 | A | 0.56 |
| 131 | C | 0.55 |
| 132 | A | 0.61 |
| 133 | B | 0.08 |
| 134 | C | 0.12 |
| 135 | C | 0.94 |
| 136 | C | N/A |
| 137 | A | 0.05 |
| 138 | B | 0.25 |
| 139 | A | 0.42 |
| 140 | B | 0.15 |
| 141 | B | 0.10 |
| 142 | B | 0.50 |
| 143 | C | 0.56 |
| 144 | A | 0.29 |
| 145 | B | 0.04 |
| 146 | C | 0.23 |
| 147 | C | 0.70 |
| 148 | C | 0.19 |
| 149 | B | 0.07 |
| 150 | C | 0.43 |
| 151 | C | 0.36 |
| 152 | B | 0.16 |
| 155 | B | 0.14 |
| 157 | B | 0.08 |
| 158 | C | 0.06 |
| 159 | B | 1.04 |
| 163 | C | 1.33 |
| 164 | C | N/A |
| 165 | C | N/A |
| 166 | C | N/A |
| 167 | C | N/A |
| 168 | C | 0.15 |
| 169 | C | 0.69 |
| 170 | C | 0.75 |
| 171 | C | 0.35 |
| 172 | C | 0.47 |
| 173 | C | 0.87 |
| 174 | C | 0.44 |
| 175 | B | 0.73 |
| 177 | A | 0.08 |
| 178 | A | 0.10 |
| 179 | A | 0.10 |
| 180 | A | 0.34 |
| 181 | A | 0.33 |
| 184 | C | 0.84 |
| 185 | B | 0.34 |
| 186 | C | 0.54 |
| 187 | B | 0.35 |
| 189 | A | 0.12 |
| 190 | C | N/A |
| 191 | C | 0.49 |
| 192 | C | 0.25 |
| 193 | C | 0.33 |
| 262 | C | 0.42 |
| 263 | C | 0.88 |
| 264 | C | 1.17 |
| 265 | C | 0.97 |
| 275 | C | 0.64 |
| 285 | C | 1.38 |

### Example B3. Foci Reduction

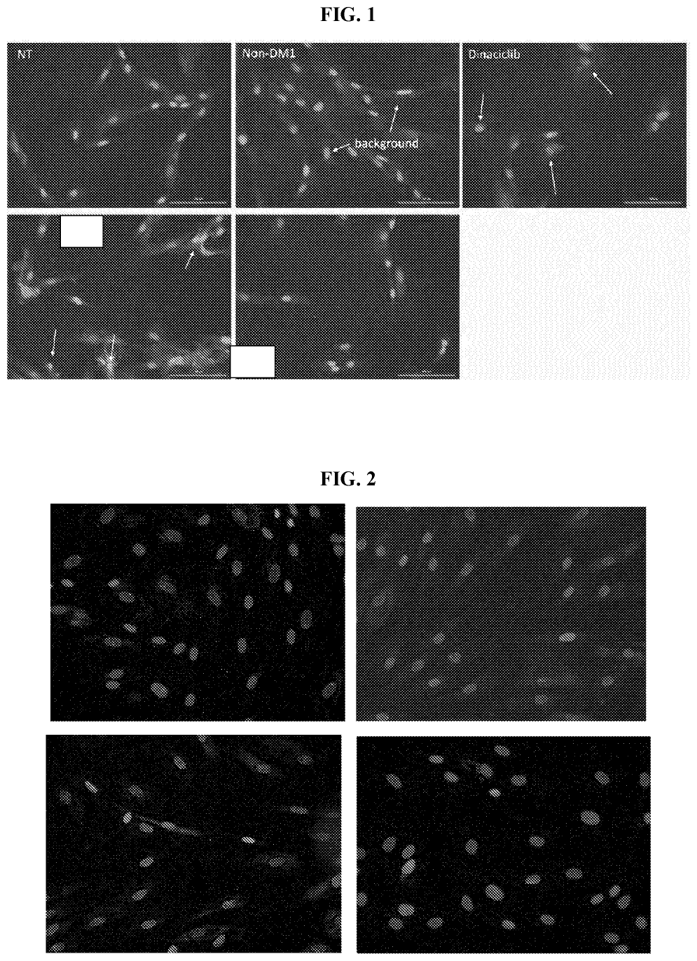

FIG. 1 shows a pictorial representation of DM1 fibroblast (1000 repeats) treatment results after 48 hrs of treatment with representative compounds of the disclosure versus with Dinaciclib or no treatment (NT). FIG. 2 shows fibroblasts after 6 days of treatment with a representative compound of the disclosure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A transcription modulator molecule having a first terminus, a second terminus, and a linker moiety, wherein:
a) the first terminus comprises a DNA-binding moiety capable of noncovalently binding to a nucleotide repeat sequence CTG or CAG;
b) the second terminus comprises a protein-binding moiety capable of binding to a regulatory molecule that modulates an expression of a gene comprising the nucleotide repeat sequence CTG or CAG; and
c) the linker moiety connecting the first terminus and the second terminus; and
wherein the first comprises the structure of Formula (A-2′), or a pharmaceutically acceptable salt or solvate thereof: 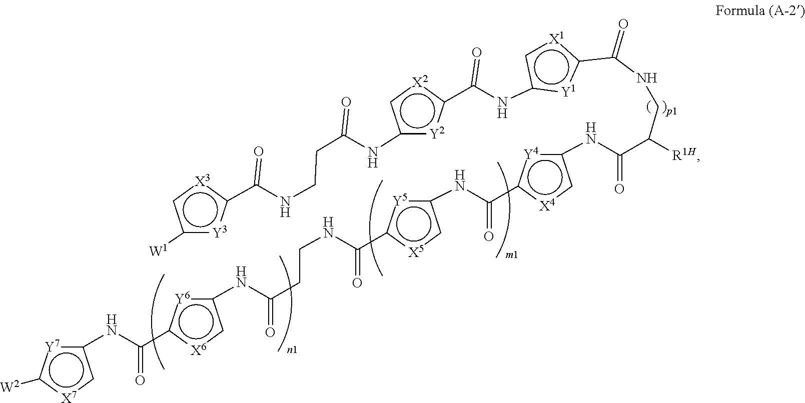
wherein:
each X¹, X², X³, X⁴, X⁵, X⁶, and X⁷ is independently O, S, or NR^{1D};
each Y¹, Y², Y³, Y⁴, Y⁵, Y⁶, and Y⁷ is independently CH or N;
W¹ is hydrogen, optionally substituted 5-10 membered heteroaryl, C₁-C₆ alkyl, —C(O)—NR^{1E}R^{1F}, —NR^{1E}—C(O)—NR^{1E}R^{1F};
W² is an optionally substituted 5-10 membered heteroaryl, C₁-C₆ alkyl, or —C(O)—NR^{1E}R^{1F};
m¹ is 0, 1, 2, or 3;
n¹ is 0, 1, 2, or 3;
p¹ is 1, 2, 3, or 4;
each R^{1D} and R^{1E} is independently hydrogen, or optionally substituted C₁-C₆ alkyl;
R^{1F} is hydrogen, an optionally substituted C₁-C₁₀ alkyl, C₁-C₁₀ heteroalkyl, PEG₁₋₂₀, or one or more AA, wherein AA is one or more amino acids selected from β-alanine, lysine, and arginine; and
R^{1H} is hydrogen, amino, cyano, or optionally C₁-C₁₀ alkyl, C₂-C₁₀ heteroalkyl.

2. The transcription modulator molecule of claim 1, or a pharmaceutically acceptable salt or solvate thereof, wherein the first terminus comprises a linear polyamide.

3. The transcription modulator molecule of claim 1 or 2, or a pharmaceutically acceptable salt or solvate thereof, wherein the polyamide is capable of binding the DNA with an affinity of less than 500 nM.

4. The transcription modulator molecule of any one of claims 1-3, or a pharmaceutically acceptable salt or solvate thereof, wherein W² is —C(O)—NR^{1E}R^{1F}.

5. The transcription modulator molecule of any one of claims 1-4, or a pharmaceutically acceptable salt or solvate thereof, wherein W² is —C(O)—(β-alanine).

6. The transcription modulator molecule of any one of claims 1-5, or a pharmaceutically acceptable salt or solvate thereof, wherein the first terminus comprises the structure of Formula (A-2), or a pharmaceutically acceptable salt, solvate, or hydrate thereof: 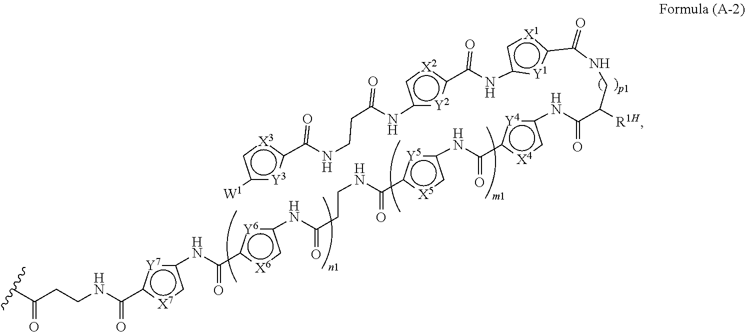
wherein:
each X¹, X², X³, X⁴, X⁵, X⁶, and X⁷ is independently O, S, or NR^{1D};
each Y¹, Y², Y³, Y⁴, Y⁵, Y⁶, and Y⁷ is independently CH or N;
W¹ is hydrogen, optionally substituted 5-10 membered heteroaryl, C₁-C₆ alkyl, —C(O)—NR^{1E}R^{1F} or —NR^{1E}—C(O)—NR^{1E}R^{1F},
m¹ is 0, 1, 2, or 3;
n¹ is 0, 1, 2, or 3;
p¹ is 1, 2, 3, or 4;
each R^{1D} and R^{1E} is independently hydrogen or C₁-C₆ alkyl; and
R^{1F} is hydrogen, an optionally substituted C₁-C₁₀ alkyl, C₂-C₁₀ heteroalkyl, PEG₁₋₂₀, or one or more AA, wherein AA is one or more amino acids selected from β-alanine, lysine, and arginine.

7. The transcription modulator molecule of claim 1 or 6, or a pharmaceutically acceptable salt or solvate thereof, wherein each X¹, X², X³, X⁴, X⁵, X⁶, and X⁷ is independently —NR^{1D}, wherein R^{1D} is C₁-C₆ alkyl.

8. The transcription modulator molecule of any one of claims 1, 6, or 7, or a pharmaceutically acceptable salt or solvate thereof, wherein each X¹, X², X³, X⁴, X⁵, X⁶, and X⁷ is independently —NCH₃.

9. The transcription modulator molecule of any one of claims 1-8, or a pharmaceutically acceptable salt or solvate thereof, wherein m¹ is 0 or 1 and n¹ is 0 or 1.

10. The transcription modulator molecule of any one of claims 1-9, or a pharmaceutically acceptable salt or solvate thereof, wherein p¹ is 2 or 3.

11. The transcription modulator molecule of any one of claims 1-10, or a pharmaceutically acceptable salt or solvate thereof, wherein W¹ is hydrogen or optionally substituted 5-10 membered heteroaryl.

12. The transcription modulator molecule of claim 11, or a pharmaceutically acceptable salt or solvate thereof, wherein the 5-10 membered heteroaryl is a pyrrole or imidazole.

13. The transcription modulator molecule of any one of claims 1-11, or a pharmaceutically acceptable salt or solvate thereof, wherein W¹ is hydrogen.

14. The transcription modulator molecule of any one of claims 1-13, or a pharmaceutically acceptable salt or solvate thereof, wherein the first terminus comprises the structure of Formula (A-3), or a pharmaceutically acceptable salt, solvate, or hydrate thereof:

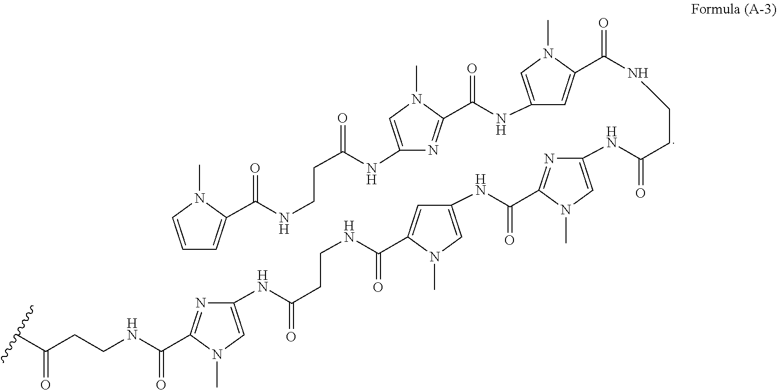 [SMILES: *C(=O)CCNC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C]

15. The transcription modulator molecule of any one of claims 1-13, or a pharmaceutically acceptable salt or solvate thereof, wherein the first terminus comprises the structure of Formula (A-4), or a pharmaceutically acceptable salt, solvate, or hydrate thereof:

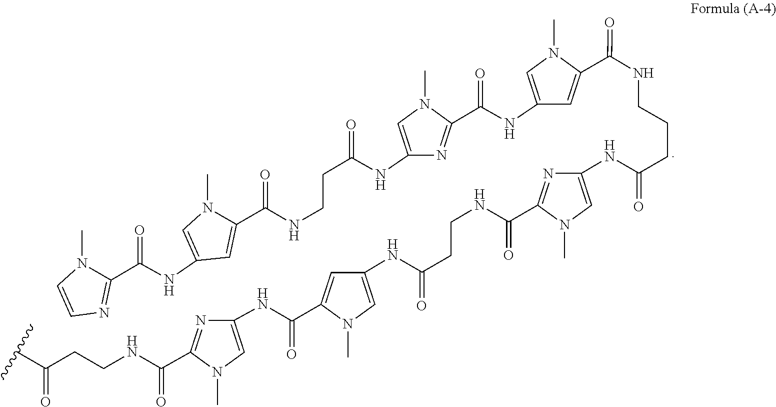 [SMILES: *C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C]

16. The transcription modulator molecule of any one of claims 1-13, or a pharmaceutically acceptable salt or solvate thereof, wherein the first terminus comprises the structure of Formula (A-5), or a pharmaceutically acceptable salt, solvate, or hydrate thereof:

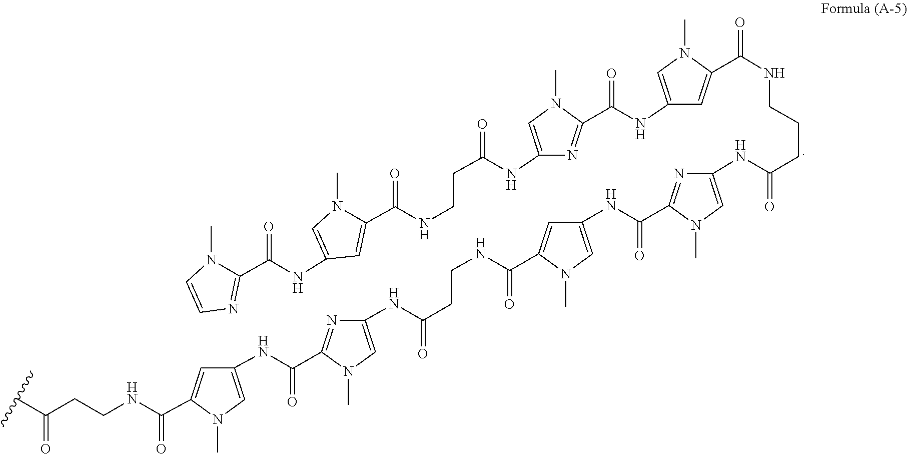 [SMILES: *C(=O)CCNC(=O)c1cc(NC(=O)c2nc(NC(=O)CCNC(=O)c3cc(NC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cn1C]

17. The transcription modulator molecule of any one of claims 1-13, or a pharmaceutically acceptable salt or solvate thereof, wherein the first terminus does not have the structure

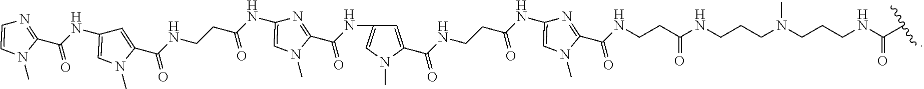 [SMILES: *C(=O)NCCCN(C)CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C ; "."]

18. The transcription modulator molecule of any one of claims 1-17, or a pharmaceutically acceptable salt or solvate thereof, wherein the linker has a length of less than about 50 Angstroms.

19. The transcription modulator molecule of any one of claims 1-17, or a pharmaceutically acceptable salt or solvate thereof, wherein the linker has a length of about 10 to 60 Angstroms.

20. The transcription modulator molecule of any one of claims 1-17, or a pharmaceutically acceptable salt or solvate thereof, wherein the linker has a length of about 20 to 40 Angstroms.

21. The transcription modulator molecule of any one of claims 1-20, or a pharmaceutically acceptable salt or solvate thereof, wherein the linker comprises a multimer having from 2 to 50 spacing moieties, and wherein the spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₋₁₂ alkyl, optionally substituted C₂₋₁₀ alkenyl, optionally substituted C₂₋₁₀ alkynyl, optionally substituted C₆₋₁₀ arylene, optionally substituted C₃₋₇ cycloalkylene, optionally substituted 5- to 10-membered heteroarylene, optionally substituted 4- to 10-membered heterocycloalkylene, an amino acid residue, —O—, —C(O)NR^{4a}—, —NR^{4a}C(O)—, —C(O)—, —NR^{4a}—, —C(O)O—, —O—, —S—, —S(O)—, —SO₂—, —SO₂NR^{4a}—, —NR^{4a}SO₂—, and —P(O)OH—, and any combinations thereof; wherein
each x is independently 2-4;
each y is independently 1-10;
each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁₋₆ alkyl.

22. The transcription modulator molecule of any one of claims 1-20, wherein the oligomeric backbone comprises -(T¹-V¹)ₐ-(T²-V²)_{b}-(T³-V³)_{c}-(T⁴-V⁴)_{d}-(T⁵-V⁵)ₑ-,
wherein a, b, c, d and e are each independently 0 or 1, and where the sum of a, b, c, d and e is 1 to 5;
T¹, T², T³, T⁴ and T⁵ are each independently selected from an optionally substituted (C₁-C₁₂) alkylene, optionally substituted alkenylene, optionally substituted alkynylene, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, a disulfide, a hydrazine, a carbohydrate, a beta-lactam, and an ester;
each m, p, and w are independently an integer from 1 to 20;
n is an integer from 1 to 30;
h is an integer from 1 to 12;
EA has the following structure: 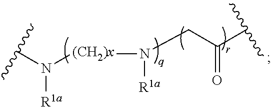
EDA has the following structure: 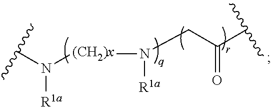
wherein each q is independently an integer from 1 to 6;
each x is independently an integer from 2 to 4 and
each r is independently 0 or 1;
(PEG)ₙ has the structure of —(CR^{2a}R^{2b}—CR^{2a}R^{2b}—O)ₙ—CR^{2a}R^{2b}—;
(modified PEG)ₙ has the structure of replacing at least one —(CR^{2a}R^{2b}—CR^{2a}R^{2b}—O)— in (PEG)ₙ with —(CH₂—CR^{2a}═CR^{2a}—CH₂—O)— or —(CR^{2a}R^{2b}—CR^{2a}R^{2b}—S)—;
AA is an amino acid residue;
V¹, V², V³, V⁴ and V⁵ are each independently selected from the group consisting of a bond, —CO—, —NR^{1a}—, —CONR^{1a}—, —NR^{1a}CO—, —CONR^{1a}C₁₋₄ alkyl-, —NR^{1a}CO—C₁₋₄ alkyl-, —C(O)O—, —OC(O)—, —O—, —S—, —S(O)—, —SO₂—, —SO₂NR^{1a}—, —NR^{1a}SO₂— and —P(O)OH—;
each R^{1a} is independently hydrogen or and optionally substituted C₁₋₆ alkyl; and each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

23. The transcription modulator molecule of claim 22, or a pharmaceutically acceptable salt or solvate thereof, wherein T¹, T², T³, T⁴, and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, an optionally substituted phenyl, piperidin-4-amino (P4A), piperidine-3-amino, piperazine, pyrrolidin-3-amino, azetidine-3-amino, para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ.

24. The transcription modulator molecule of claim 22, or a pharmaceutically acceptable salt or solvate thereof, wherein piperidin-4-amino (P4A) is 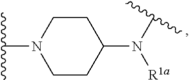
wherein R^{1a} is H or C₁₋₆ alkyl.

25. The transcription modulator molecule of claim 22, or a pharmaceutically acceptable salt or solvate thereof, wherein T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, and optionally substituted 5-10 membered heteroarylene.

26. The transcription modulator molecule of any one of claims 1-25, or a pharmaceutically acceptable salt or solvate thereof, wherein the linker comprises —N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

27. The transcription modulator molecule of any one of claims 1-25, or a pharmaceutically acceptable salt or solvate thereof, wherein the linker comprises —(CH₂—C(O)N(R″)—(CH₂)_{q}—N(R′)—(CH₂)_{q}—N(R″)C(O)—(CH₂)ₓ—C(O)N(R″)-A-, —(CH₂)ₓ—C(O)N(R″)—(CH₂CH₂O)_{y}(CH₂)ₓ—C(O)N(R″)-A-, —C(O)N(R″)—(CH₂)_{q}—N(R′)—(CH₂)_{q}—N(R″)C(O)—(CH₂)ₓ-A-, —(CH₂)ₓ—O—(CH₂CH₂O)_{y}—(CH₂)ₓ—N(R″)C(O)—(CH₂)ₓ-A-, or —N(R″)C(O)—(CH₂)—C(O)N(R″)—(CH₂)ₓ—O(CH₂CH₂O)_{y}(CH₂)ₓ-A-; wherein R′ is methyl; R″ is hydrogen; each x and y are independently an integer from 1 to 10; each q is independently an integer from 2 to 10; and each A is independently selected from a bond, an optionally substituted C₁₋₁₂ alkyl, an optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

28. The transcription modulator molecule of any one of claims 1-27, or a pharmaceutically acceptable salt or solvate thereof, wherein the linker comprises a structure of Formula (C-1): 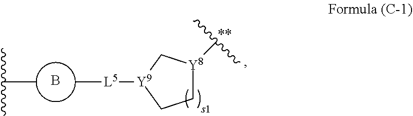
wherein,
Ring B is absent, arylene or heterocycloalkylene;
L⁵ is absent, optionally substituted alkylene or alkenylene;
each Y⁸ and Y⁹ is independently CH or N;
s¹ is 0-3; and
** denotes attachment to the second terminus.

29. The transcription modulator molecule of claim 28, or a pharmaceutically acceptable salt or solvate thereof, wherein the linker comprises a structure of Formula (C-2): 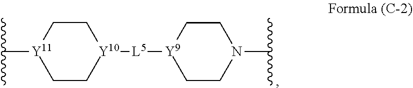
wherein each Y¹⁰ and Y¹¹ is independently N or CH.

30. The transcription modulator molecule of claim 28, or a pharmaceutically acceptable salt or solvate thereof, wherein the linker comprises a structure of Formula (C-3): 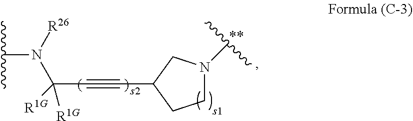
wherein,
s1 is 0-3;
s2 is 1-3;
R²⁶ is an optionally substituted C₁₋₂₀ alkylene or heteroalkylene;
each R^{IG} is independently hydrogen or C₁-C₃ alkyl; and
** denotes attachment to the second terminus.

31. The transcription modulator molecule of any one of claims 1-30, or a pharmaceutically acceptable salt or solvate thereof, wherein the linker is joined with the first terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR^{1a}SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene; wherein each x and y are independently 1-4, and each R^{1a} is independently a hydrogen or optionally substituted C₁₋₆ alkyl.

32. The transcription modulator molecule of any one of claims 1-31, or a pharmaceutically acceptable salt or solvate thereof, wherein the linker is joined with the first terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl.

33. The transcription modulator molecule of any one of claims 1-32, or a pharmaceutically acceptable salt or solvate thereof, wherein the second terminus comprises a moiety capable of binding to the regulatory protein, and the moiety is from a compound capable of binding to the regulatory protein.

34. The transcription modulator molecule of any one of claims 1-33, or a pharmaceutically acceptable salt or solvate thereof, wherein the second terminus is selected from a bromodomain inhibitor, a BPTF inhibitor, a methylcytosine dioxygenase inhibitor, a DNA demethylase inhibitor, a helicase inhibitor, an acetyltransferase inhibitor, a histone deacetylase inhibitor, a CDK-9 inhibitor, a positive transcription elongation factor inhibitor, and a polycomb repressive complex inhibitor.

35. The transcription modulator molecule of claim 34, or a pharmaceutically acceptable salt or solvate thereof, wherein the second terminus is a CDK9 inhibitor

36. The transcription modulator molecule of claim 34, or a pharmaceutically acceptable salt or solvate thereof, wherein the second terminus is selected from CDK9i, CDK7i, CDK12/13i, Pan-CDKi, a L3MBTL3 recruiter, a CBX recruiter or an EED recruiter.

37. The transcription modulator molecule of claim 34, or a pharmaceutically acceptable salt or solvate thereof, wherein the second terminus is a recruiter of PRC1 or PRC2.

38. The transcription modulator molecule of any one of claims 1-33, or a pharmaceutically acceptable salt or solvate thereof, wherein that the second terminus is not a Brd4 binding moiety.

39. The transcription modulator molecule of any one of claims 1-33, wherein the second terminus comprises a compound of Formula (C), or a pharmaceutically acceptable salt or solvate thereof: 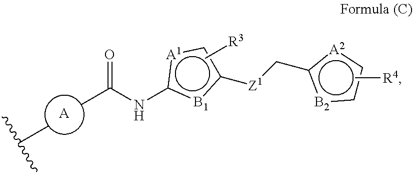
wherein,
Ring A is a 5-10 membered heteroaryl or heterocycloalkyl;
A¹ and A² are each independently CH or N;
B¹ and B² are each independently O, S, or NR⁵;
Z¹ is O, S, or NR⁵;
each R³ and R⁴ is independently hydrogen, halogen, or C₁-C₆ alkyl; and
R⁵ is hydrogen or C₁-C₆ alkyl.

40. The transcription modulator molecule of claim 39, wherein the second terminus comprises a compound of Formula (C-1), or a pharmaceutically acceptable salt or solvate thereof:

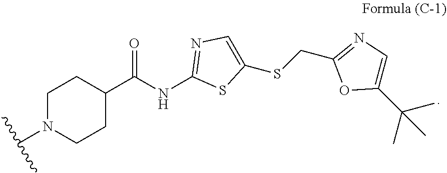 [SMILES: *N1CCC(C(=O)Nc2ncc(SCc3ncc(C(C)(C)C)o3)s2)CC1 ; "."]

41. The transcription modulator molecule of any one of claims 1-33, wherein the second terminus comprises a compound of Formula (D), or a pharmaceutically acceptable salt or solvate thereof: 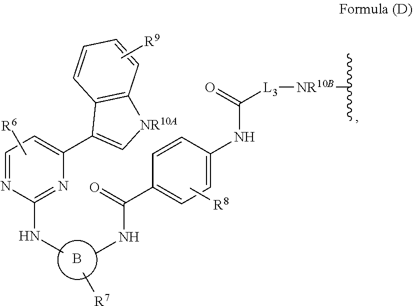
wherein,
L₃ is optionally substituted alkylene or heteroalkylene;
each R⁶, R⁷, R⁸, and R⁹ is independently a hydrogen, halogen, optionally substituted C₁₋₆ alkyl, C₁₋₆ haloalkyl, or C₁₋₆ hydroxyalkyl; and
R^{10A} is hydrogen, C₁-C₆ alkyl, or SO₂—R^{10C};
R^{10B} is independently hydrogen or C₁-C₆ alkyl; and
R^{10C} is C₁-C₆ alkyl or aryl.

42. The transcription modulator molecule of claim 41, wherein the second terminus comprises a compound of Formula (D-1), or a pharmaceutically acceptable salt or solvate thereof:

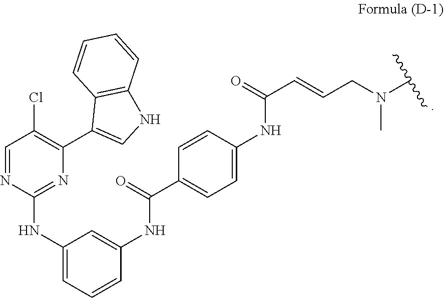 [SMILES: *N(C)C/C=C/C(=O)Nc1ccc(C(=O)Nc2cccc(Nc3ncc(Cl)c(-c4c[nH]c5ccccc45)n3)c2)cc1 ; "."]

43. The transcription modulator molecule of claim 41, wherein the second terminus comprises a compound of Formula (D-2), or a pharmaceutically acceptable salt or solvate thereof:

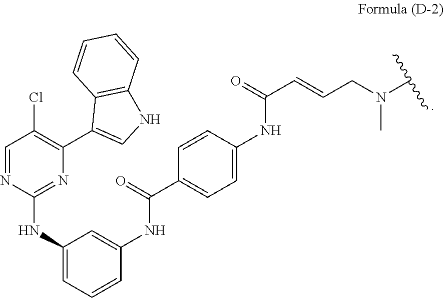

44. The transcription modulator molecule of any one of claims 1-33, wherein the second terminus comprises a compound of Formula (E), or a pharmaceutically acceptable salt or solvate thereof: 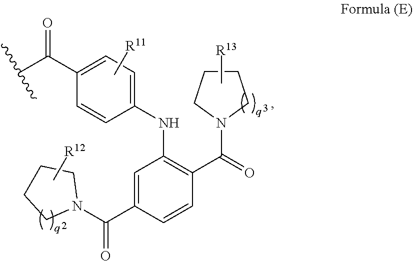
wherein,
each q2 and q3 is independently 1, 2, 3, or 4;
R¹¹ is hydrogen, halogen, an optionally substituted C₁₋₆ alkyl, C₁₋₆ haloalkyl, or C₁₋₆ hydroxyalkyl; and
each R¹² and R¹³ is independently an optionally substituted 5-8 membered heterocycloalkyl.

45. The transcription modulator molecule of claim 44, wherein the second terminus comprises a compound of Formula (E-1), or a pharmaceutically acceptable salt or solvate thereof:

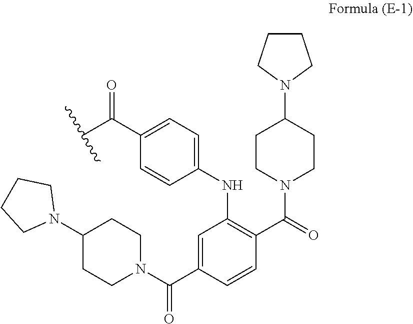 [SMILES: *C(=O)c1ccc(Nc2cc(C(=O)N3CCC(N4CCCC4)CC3)ccc2C(=O)N2CCC(N3CCCC3)CC2)cc1]

46. The transcription modulator molecule of any one of claims 1-33, wherein the second terminus comprises a compound of Formula (F), or a pharmaceutically acceptable salt or solvate thereof: 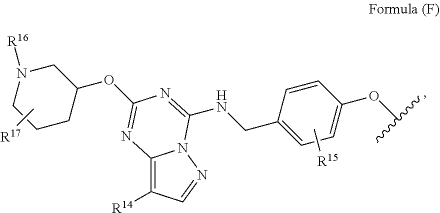
wherein,
each R¹⁴ and R¹⁷ is independently hydrogen, halogen, optionally substituted C₁₋₆ alkyl, C₁₋₆ haloalkyl, or C₁₋₆ hydroxyalkyl;
R¹⁵ is an optionally substituted 5 membered heteroaryl; and
R¹⁶ is hydrogen or C₁-C₆ alkyl.

47. The transcription modulator molecule of claim 46, wherein the second terminus comprises a compound of Formula (F-1), or a pharmaceutically acceptable salt or solvate thereof:

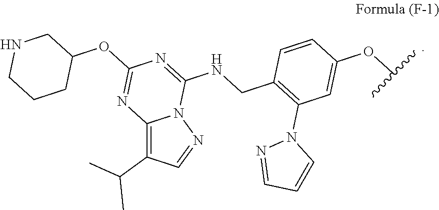 [SMILES: *Oc1ccc(CNc2nc(OC3CCCNC3)nc3c(C(C)C)cnn23)c(-n2cccn2)c1]

48. The transcription modulator molecule of any one of claims 1-33, wherein the second terminus comprises a compound of Formula (G), or a pharmaceutically acceptable salt or solvate thereof: 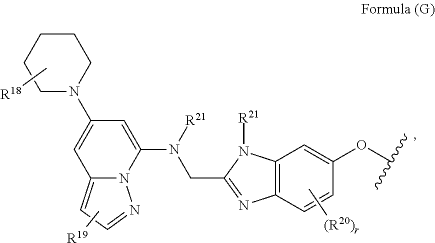
wherein,
r is 0, 1, or 2;
R¹⁸ is hydrogen, optionally substituted C₁₋₆ alkyl, C₁₋₆ haloalkyl, or C₁₋₆ hydroxyalkyl;
R¹⁹ is hydrogen, halogen, or an optionally substituted C₁₋₆ alkyl.
each R²⁰ is independently hydrogen, halogen, or C₁-C₆ alkyl; and
each R²¹ is independently hydrogen or C₁-C₆ alkyl.

49. The transcription modulator molecule of claim 48, wherein the second terminus comprises a compound of Formula (G-1), or a pharmaceutically acceptable salt or solvate thereof:

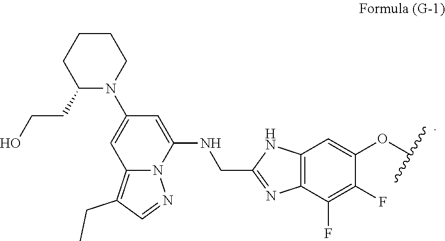 [SMILES: *Oc1cc2[nH]c(CNc3cc(N4CCCC[C@H]4CCO)cc4c(CC)cnn34)nc2c(F)c1F]

50. The transcription modulator molecule of any one of claims 1-33, wherein the second terminus comprises a compound of Formula (H-1), or a pharmaceutically acceptable salt or solvate thereof:

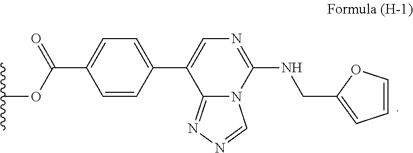 [SMILES: *OC(=O)c1ccc(-c2cnc(NCc3ccco3)n3cnnc23)cc1]

51. The transcription modulator molecule of any one of claims 1-33, wherein the second terminus comprises a compound of Formula (H-2), or a pharmaceutically acceptable salt or solvate thereof:

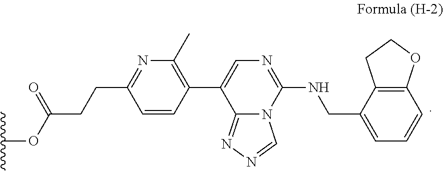 [SMILES: *OC(=O)CCc1ccc(-c2cnc(NCc3cccc4c3CCO4)n3cnnc23)c(C)n1]

52. The transcription modulator of any one of claims 1-33, wherein the second terminus comprises a compound of Formula (J), or a pharmaceutically acceptable salt or solvate thereof: 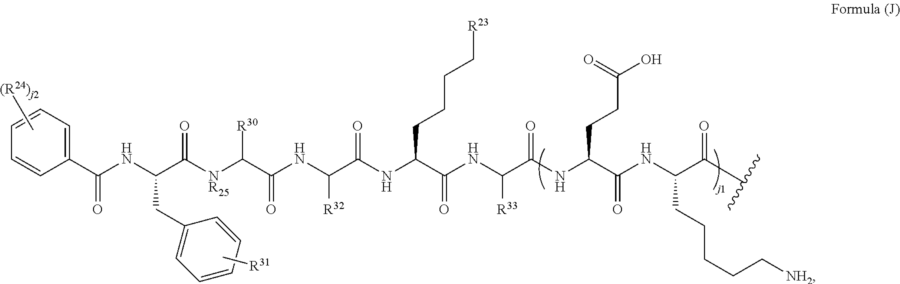
wherein,
R²³ is —NR^{23A}R^{23B} or —NR^{23A}(R^{23B})₂; wherein
R^{23A} and R^{23B} are each independently an optionally substituted C₁₋₆ alkyl, C₃-C₁₀ cycloalkyl, aryl or heteroaryl; or
R^{23A} and R^{23B} are joined together with the nitrogen to which they are attached to form a heterocyclic ring;
R²⁴ is hydrogen, halogen, C₁₋₆ alkyl, C₁₋₆ haloalkyl, or C₁₋₆ alkoxy;
R²⁵ is hydrogen or C₁₋₃ alkyl;
R³⁰, R³², and R³³ are each independently hydrogen, halogen, optionally substituted C₁₋₆ alkyl, C₁₋₆ alkoxy, or C₃₋₆ cycloalkyl;
R³¹ is C₁₋₆ alkyl or C₃₋₁₀ cycloalkyl;
j₁ is 0 or 1; and
j₂ is 0, 1, 2, or 3.

53. The transcription modulator molecule of claim 52, wherein the second terminus comprises a compound of Formula (J-1), or a pharmaceutically acceptable salt or solvate thereof:

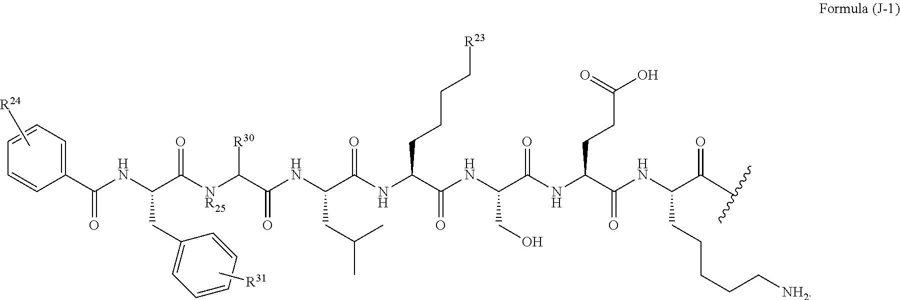

54. The transcription modulator molecule of claim 52, wherein the second terminus comprises a compound of Formula (J-2), or a pharmaceutically acceptable salt or solvate thereof:

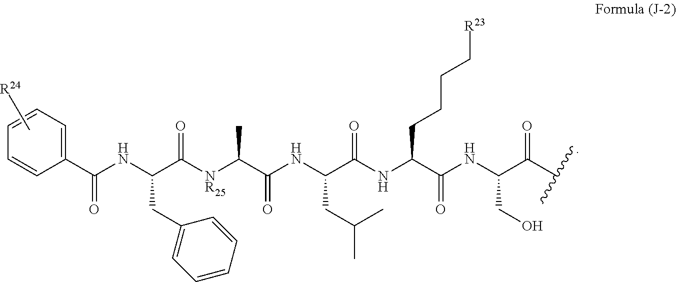

55. The transcription modulator molecule of claim 52, wherein the second terminus comprises a compound of Formula (J-3), or a pharmaceutically acceptable salt or solvate thereof:

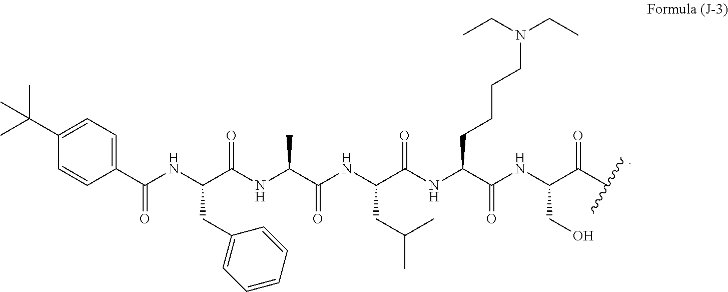 [SMILES: *C(=O)[C@H](CO)NC(=O)[C@H](CCCCN(CC)CC)NC(=O)[C@H](CC(C)C)NC(=O)[C@H](C)NC(=O)[C@H](Cc1ccccc1)NC(=O)c1ccc(C(C)(C)C)cc1]

56. The transcription modulator molecule of claim 52, wherein the second terminus comprises a compound of Formula (J-4), or a pharmaceutically acceptable salt or solvate thereof:

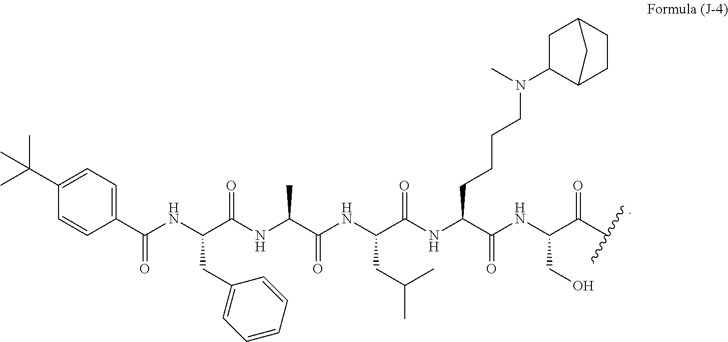 [SMILES: *C(=O)[C@H](CO)NC(=O)[C@H](CCCCN(C)C1CC2CCC1C2)NC(=O)[C@H](CC(C)C)NC(=O)[C@H](C)NC(=O)[C@H](Cc1ccccc1)NC(=O)c1ccc(C(C)(C)C)cc1]

57. The transcription modulator molecule of any one of claims 1-33, wherein the second terminus comprises a compound of Formula (J-5), or a pharmaceutically acceptable salt or solvate thereof: 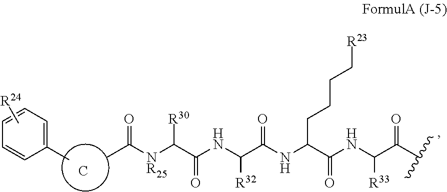
wherein,
Ring C is an optionally substituted 5 to 6 membered heterocyclyl ring;
R²³ is —NR^{23A}R^{23B} or —NR^{23A}(R^{23B})₂; wherein
R^{23A} and R^{23B} are each independently an optionally substituted C₁₋₆ alkyl, C₃-C₁₀ cycloalkyl, aryl or heteroaryl; or
R^{23A} and R^{23B} are joined together with the nitrogen to which they are attached to form a heterocyclic ring;
R²⁴ is hydrogen, halogen, C₁₋₆ alkyl, C₁₋₆ haloalkyl, or C₁₋₆ alkoxy;
R²¹ is hydrogen or C₁₋₃ alkyl; and
R³⁰, R³², and R³³ are each independently hydrogen, halogen, optionally substituted C₁₋₆ alkyl, C₁₋₆ alkoxy, or C₃-C₆ cycloalkyl ring.

58. The transcription modulator molecule of claim 57, wherein the second terminus comprises a compound of Formula (J-6), or a pharmaceutically acceptable salt or solvate thereof:

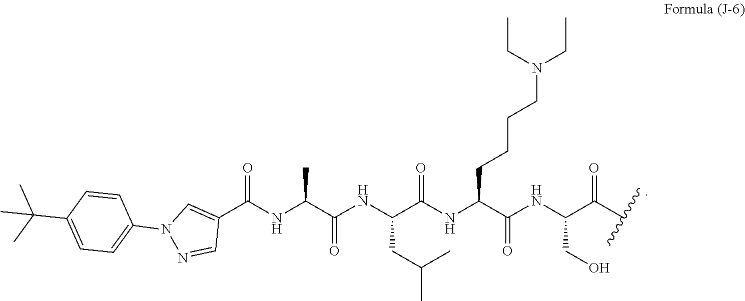 [SMILES: *C(=O)[C@H](CO)NC(=O)[C@H](CCCCN(CC)CC)NC(=O)[C@H](CC(C)C)NC(=O)[C@H](C)NC(=O)c1cnn(-c2ccc(C(C)(C)C)cc2)c1]

59. The transcription modulator molecule of any one of claims 1-33, wherein the second terminus comprises a compound of Formula (J-7), or a pharmaceutically acceptable salt or solvate thereof:

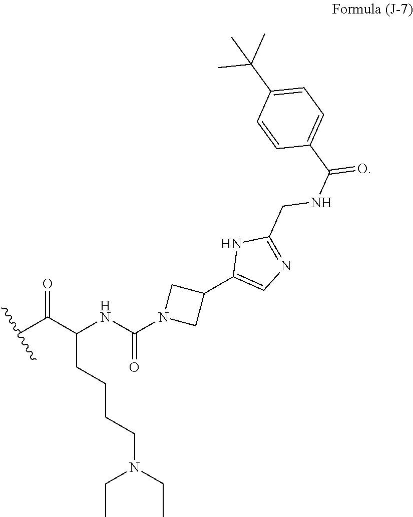 [SMILES: *C(=O)C(CCCCN(CC)CC)NC(=O)N1CC(c2cnc(CNC(=O)c3ccc(C(C)(C)C)cc3)[nH]2)C1]

60. The transcription modulator molecule of any one of claims 1-33, wherein the second terminus comprises a compound of Formula (K), or a pharmaceutically acceptable salt or solvate thereof: 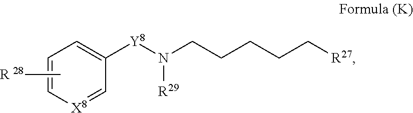
wherein,
X⁸ is CH or N;
Y⁸ is —C(O)—, or —S(O)₂—;
R²⁷ is an optionally substituted cation of C₁₋₆ alkyl, C₃-C₁₀ cycloalkyl, or 5 to 10-membered heteroaryl;
R²⁸ is hydrogen, halogen, or C₁₋₆ alkyl; and
R²⁹ is hydrogen or C₁₋₃ alkyl.

61. The transcription modulator molecule of any one of claims 1-33, wherein the second terminus comprises a compound selected from: 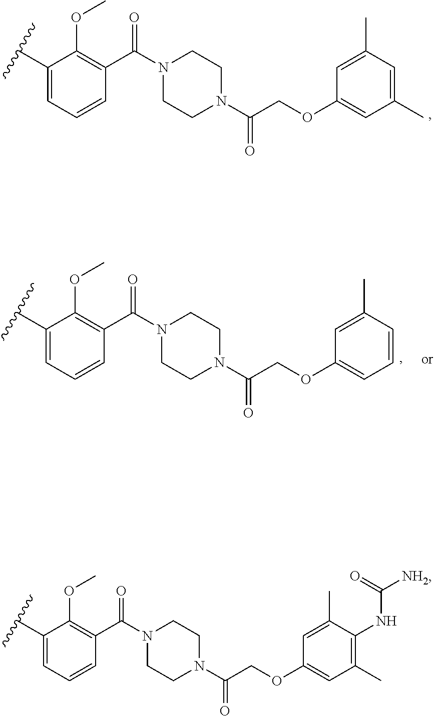 [SMILES: *c1cccc(C(=O)N2CCN(C(=O)COc3cc(C)cc(C)c3)CC2)c1OC ; *c1cccc(C(=O)N2CCN(C(=O)COc3cccc(C)c3)CC2)c1OC ; *c1cccc(C(=O)N2CCN(C(=O)COc3cc(C)c(NC(N)=O)c(C)c3)CC2)c1OC]
or a pharmaceutically acceptable salt or solvate thereof.

62. A pharmaceutical composition comprising a transcription modulator molecule of any one of claims 1-61, or a pharmaceutically acceptable salt or solvate thereof, and a pharmaceutically acceptable carrier.

63. A method of modulation of the expression of a dmpk, comprising contacting the dmpk with a transcription modulator molecule of any one of claims 1-61, or a pharmaceutically acceptable salt or solvate thereof, or pharmaceutical composition of claim 62.

64. A method of treatment of a disease or condition caused by overexpression of a dmpk in a subject in need thereof, comprising administering to the subject a therapeutically effective amount of a transcription modulator molecule of one of claims 1-61, or a pharmaceutically acceptable salt or solvate thereof or a pharmaceutically acceptable composition of claim 62.

65. The method of claim 64, wherein the disease or condition is myotonic dystrophy type 1 (DM1).

66. A method of treating Fuchs' Endothelial Corneal Dystrophy (FECD) in a subject in need thereof, the method comprising administering to the subject a therapeutically effective amount of a transcription modulator molecule of any one of claims 1-61, or a pharmaceutically acceptable salt or solvate thereof or a pharmaceutical composition of claim 62.

67. The method of any one of claims 63-66, comprising administering an additional therapeutic agent.
